(19) United States
(12) Patent Application Publication
(10) Pub. No.: US 2025/0276031 A1
(43) Pub. Date: Sep. 4, 2025

(54) PRODRUGS AS SELECTIVE ANTIBIOTICS AGAINST COLIBACTIN-PRODUCING E. COLI

(71) Applicants: New York University in Abu Dhabi Corporation, Abu Dhabi (AE); Max-Planck-Gesellschaft zur Foerderung der Wissenschaften e.V., München (DE)

(72) Inventors: Alan Healy, Abu Dhabi (AE); Helge Björn Bode, Marburg (DE)

(21) Appl. No.: 19/066,637

(22) Filed: Feb. 28, 2025

Related U.S. Application Data
(60) Provisional application No. 63/559,836, filed on Feb. 29, 2024.

(51) Int. Cl. A61K 38/06; A61P 31/04

(52) U.S. Cl. CPC A61K 38/06; A61P 31/04

(57) ABSTRACT

The present disclosure provides methods of preventing colorectal cancer and/or killing colibactin-producing E. coli. Also provided are prodrug antibiotics suitable for preventing colorectal cancer and/or killing colibactin-producing E. coli, and methods of making prodrug antibiotics. Various prodrug antibiotics may be used. For example, a prodrug antibiotic may be:

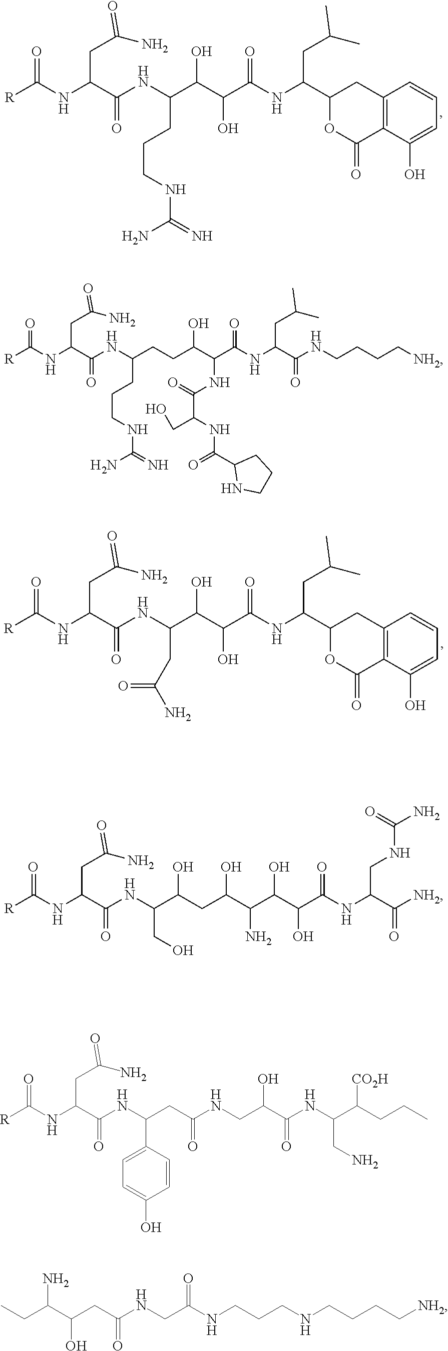

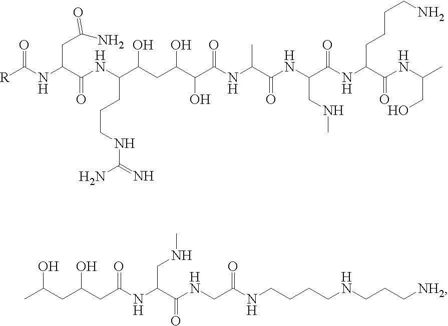

or any combination thereof, wherein R is a substituted or unsubstituted aliphatic group. In various examples, the prodrug antibiotic may be a salt or in the form of a composition.

### CROSS REFERENCE TO RELATED APPLICATION

This application claims priority to U.S. Provisional Patent Application No. 63/559,836, filed Feb. 29, 2024, the disclosure of which is incorporated herein by reference.

### SEQUENCE LISTING

The instant application contains a Sequence Listing, which has been submitted in .xml format and is hereby incorporated by incorporated by reference in its entirety. Said .xml copy was created on Feb. 27, 2025, is named “058636_00790_ST26.xml”, and is 45,494 bytes in size.

### BACKGROUND OF THE DISCLOSURE

pks⁺ E. coli is a select strain of commensal bacteria that produces a genotoxin called colibactin. This bacterium has been linked with the formation and progression of colitis, IBS and colorectal cancer (CRC). pks⁺E. coli are distinguished by the presence of the biosynthetic enzymes used to produce colibactin. The E. coli strain produces the toxin as an inactive “prodrug” form termed precolibactin. This gets converted to the active colibactin by a dedicated peptidase (ClbP) just before release from the bacteria to prevent autotoxicity.

### SUMMARY OF THE DISCLOSURE

The present disclosure provides methods of preventing colorectal cancer and/or killing colibactin-producing E. coli. Also provided are prodrug antibiotics suitable for preventing colorectal cancer and/or killing colibactin-producing E. coli, and methods of making prodrug antibiotics.

In an aspect, the present disclosure provides various methods. The methods may be methods for killing E. coli that product colibactin (ClbP) and/or preventing colorectal cancer in a subject and/or treating colorectal cancer. The methods may also be suitable to prevent or treat other conditions associated with ClbP producing E. coli. In various examples, the method may kill any bacteria that expresses ClbP protease.

A method may be for killing a least a portion of an E. coli population, where at least some or all the E. coli expresses ClbP. The method may comprise contacting at least a portion of the E. coli with a prodrug antibiotic at a therapeutically effective amount of the resulting drug. Following contact with the E. coli, the prodrug is cleaved to form a drug, resulting in the death of at least some of the ClbP-expressing E. coli.

In various examples, the prodrug antibiotics of the present disclosure selectively kill strains of Clb⁺ E. coli, while not affecting other strains of E. coli present. In various embodiments, a method of the present disclosure may also prevent in cancer initiation of colorectal cancer in subjects having colitis, irritable bowel syndrome, or the like. Similarly, without intending to be bound by any particular theory, it is considered that the method may also be used in treating other conditions and diseases associated with Clb⁺ E. coil.

Various prodrug antibiotics may be used. For example, a prodrug antibiotic may be

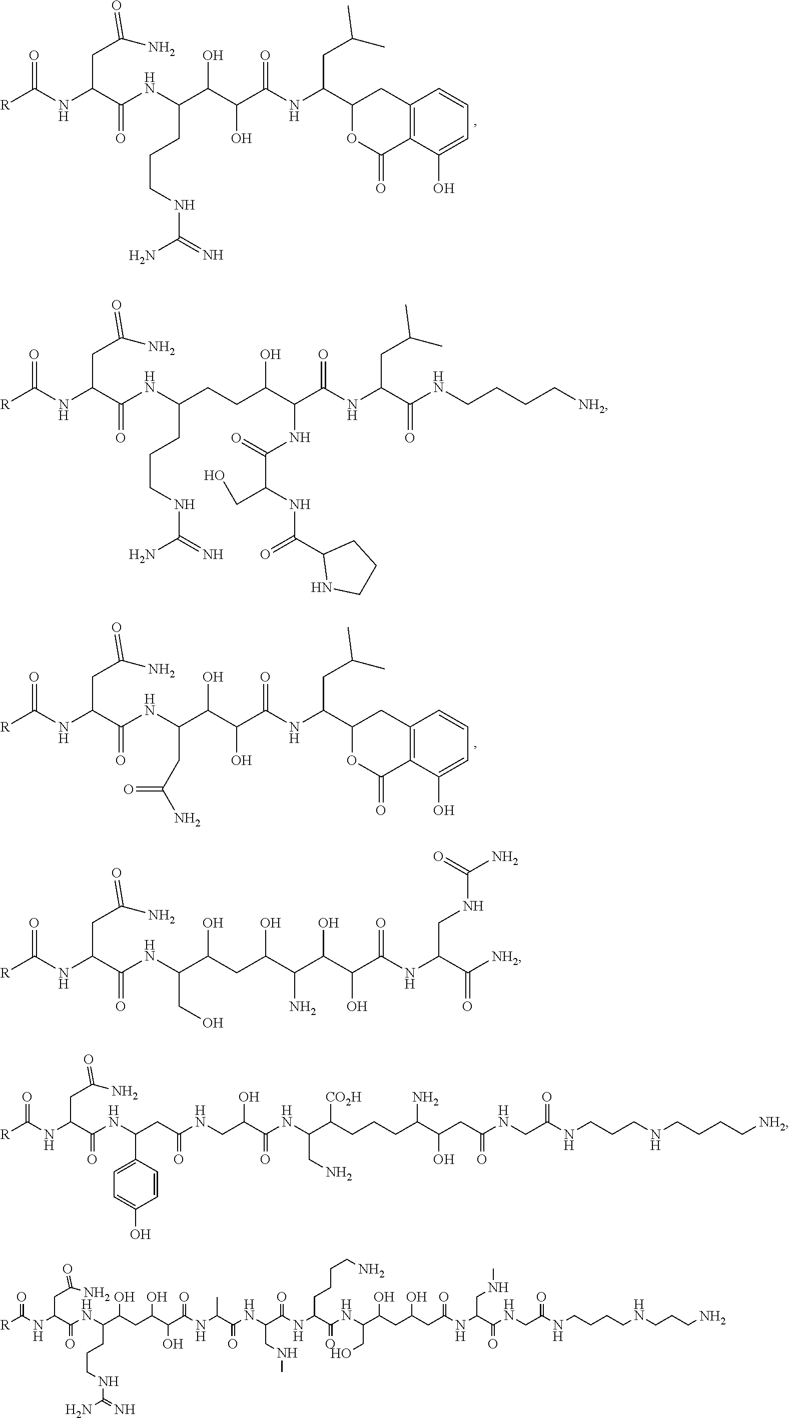

or any combination thereof, wherein R is a substituted or unsubstituted aliphatic group. In various examples, the prodrug antibiotic may be a salt or in the form of a composition.

In various examples, the subject in need of treatment may be genetically predisposed to colorectal cancer and/or the subject has colitis and/or irritable bowel syndrome (IBS) and/or an inflammatory bowel disorder. In various examples, the administration may be for the purpose of prophylaxis. In various other examples, the administration may be to partially or completely cure an individual having colorectal cancer and/or the subject has colitis and/or irritable bowel syndrome (IBS) and/or an inflammatory bowel disorder.

In various examples, a prodrug antibiotic of the present disclosure may be used in the treatment of colorectal cancer in combination with other forms of treatment. For example, the other form of treatment may be radiation and/or chemotherapy and/or other known cancer treatment regimes. Various chemotherapy agents (e.g., one or more different chemotherapy agents) may be used in combination with a prodrug antibiotic of the present disclosure.

In an aspect, the present disclosure provides compounds. A compound of the present disclosure may have the following structure:

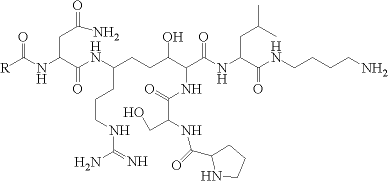

or a salt thereof, wherein R is a substituted or unsubstituted aliphatic group, with the proviso the compound does not have the following structure:

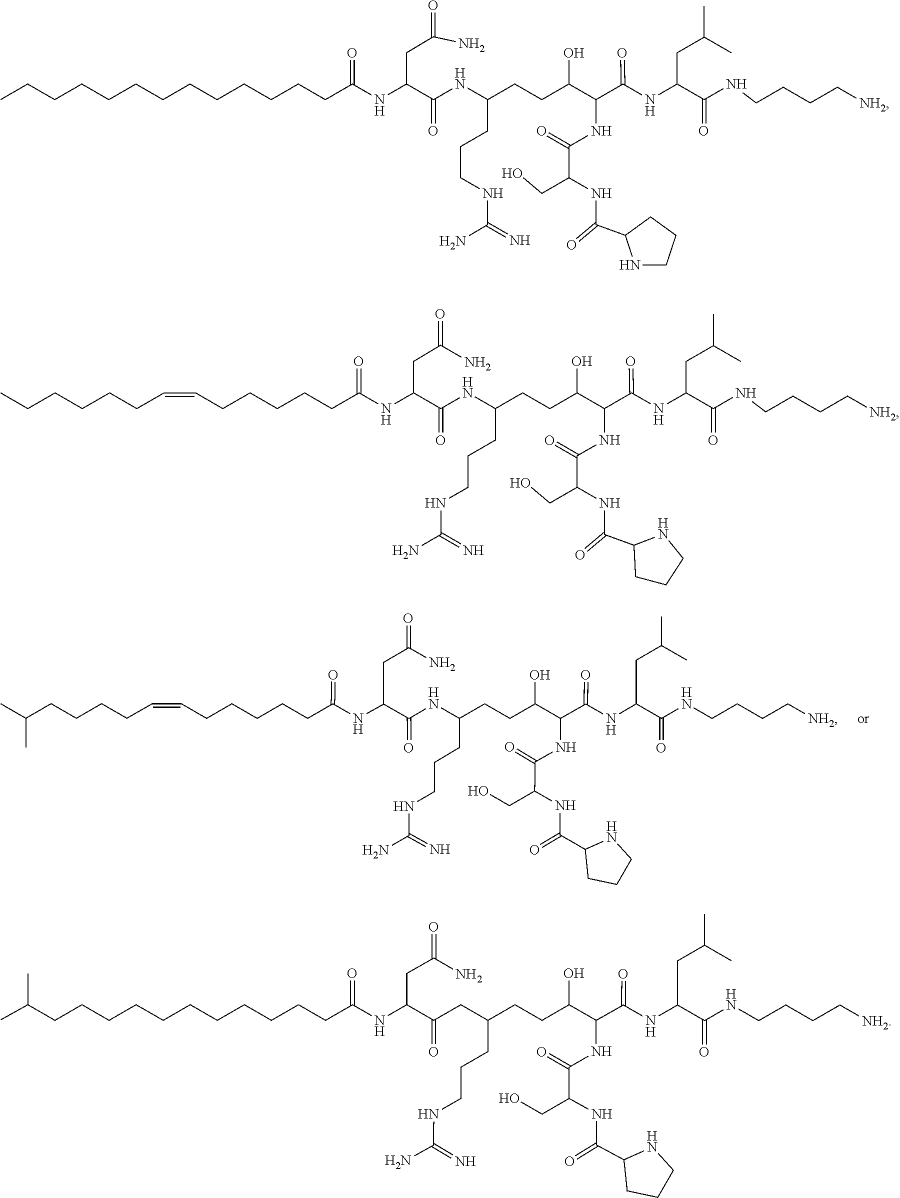

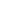

In an aspect, the present disclosure provides compositions comprising one or more compound(s) of the present disclosure. The compositions may further comprise one or more pharmaceutically acceptable carrier(s).

The compositions may include one or more pharmaceutically acceptable carrier(s). The composition may be for administration to a subject in need of treatment.

### BRIEF DESCRIPTION OF THE FIGURES

For a fuller understanding of the nature and objects of the disclosure, reference should be made to the following detailed description taken in conjunction with the accompanying figures.

FIG. 1 shows a cartoon depicting a carton of colibactin formation via ClbP cleavage.

FIG. 2 shows a cartoon depicting antibiotic formation via ClbP cleavage.

FIG. 3 shows prexenocoumacin (PXCNA), an inactive prodrug antibiotic can be taken up and the prodrug cleaved in E. coli expressing the ClbP protease.

FIG. 4 shows various prodrug antibiotics.

FIG. 5 shows various test peptides used in cultures testing ClbP activity.

FIG. 6 shows cleavage data (LC-MS) from ClbP activity with D-Asn L-Arg test peptide.

FIG. 7 shows cleavage data (LC-MS) from ClbP activity with L-Asn L-Arg test peptide.

FIG. 8 shows extracted ion chromatogram (EIC) cleavage data (LC-MS) from ClbP activity with I.-Asn I.-Arg test peptide.

FIG. 9 shows cleavage data (LC-MS) from ClbP activity with D-Asn L-Ala test peptide.

FIG. 10 shows cleavage data (LC-MS) from ClbP activity with L-Ala L-Ala test peptide.

FIG. 11 shows cleavage data (LC-MS) from ClbP activity with D-Asn L-Val test peptide.

FIG. 12 shows cleavage data (LC-MS) from ClbP activity with D-Asn D-Asn test peptide.

FIG. 13 shows a summary of substrate specificity with ClbP.

FIG. 14 shows PXCNA (10 mg/L) activity with ClbP.

FIG. 15 shows PXCNA (25 mg/L) activity with ClbP.

FIG. 16 shows PXCNA (50 mg/L) activity with ClbP.

FIG. 17 shows PXCNA (10 mg/L) activity with XcnG.

FIG. 18 shows a cartoon depicting how PXCNA activity was measured using a TecanSpark® reader.

FIG. 19 shows fluorescence assay parameters used.

FIG. 20 shows results for when assaying effect of PXCNA on E. coli C41 DE3 pET29b clbP pSEVA631 mGFP and E. coli C41 DE3 pET29b pSEVA mScarlet-i separately.

FIG. 21 shows results for when assaying effect of PXCNA on E. coli C41 DE3 pET29b_clbP pSEVA631_mGFP and E. coli C41 DE3 pET29b pSEVA_mScarlet-i separately.

FIG. 22 shows results for a co-culture competition assay.

FIG. 23 shows results for a minimum inhibitory concentration (MIC) assay of xenocoumacin 1.

FIG. 24 shows results for a minimum inhibitory concentration (MIC) assay of xenocoumacin 1.

FIG. 25 shows results for a minimum inhibitory concentration (MIC) assay of xenocoumacin 1.

FIG. 26 shows a synthetic scheme for prerhabdobranin.

FIG. 27 shows an overview of the gene organization of three different rhabdobranin BGC types (rdbl, 2 and 3), encoded in a Photorhabdus and various Xenorhabdus strains, with each gene class depicted in a specific color. Additionally, the domain architecture of the encoded NRPS and PKS is shown (NRPS: Activation domain (A), peptidyl carrier protein (PCP), condensation domain (C), epimerization domain (E), thioesterase domain (TE); PKS: Acyltransferase domain (AT), β-ketosynthase domain (KS), acyl carrier protein (ACP), ketoreductase domain (KR), dehydratase domain (DH), and enoylreductase domain (ER)) (Modified from: (Desalegne Abebew Syit 2021)).

FIG. 28 shows a proposed mechanism of the rhabdobranin biosynthesis in Xenorhabdus budapestensis DSM 16342, with the RdbP peptidase catalyzing the activation of pre-rhabdobranin A to D by the release of the N-terminal acylated D asparaginyl moiety (Modified from: (Shi et al. 2022b)).

FIG. 29 shows HPLC-HR-MS analysis of Xenorhabdus budapestensis DSM 16342 pCEP_kan-rdbA, either 0.4% L-arabinose induced or non-induced (dashed line) for the detection of rhabdobranin I (1). A C18 column separation, showing extracted ion chromatograms (EICs) detecting rhabdobranin I at a retention time of 0.53 min. B Amide column for HILIC separation, showing EICs detecting rhabdobranin I at a retention time of 3.59 min. (Structure was predicted in consideration of HR-MS data (Table 5).

FIG. 30 shows a Large-scale in vitro conversion of pre-rhabdobranin I A, B, C, and D (2, 3, 4 and 5) isolated from a Xenorhabdus budapestensis DSM 16342 production culture. A Depiction of rhabdobranin BGC ArdbP type I gene organization with promoter exchange. B Structures and HR-MS data of the in vitro converted pre-rhabdobranin I A, B, C and D (EICs of compound 2-5) containing fraction to the resulting rhabdobranin I (1) (BPC: Base peak chromatogram) (The rhabdobranin I (1) structure was verified by NMR spectroscopy (FIG. 42-47, Table 6).

FIG. 31 shows antimicrobial activity of rhabdobranin I (1) and synthetic pre-rhabdobranin B (3) against Bacillus subtilis B168, Escherichia coli MG1655, Micrococus luteus, and Saccharomyces cerevisiae CEN.PK2. The disc diffusion assay was performed by applying either 5 μL (1), 10 μL (2) or 20 μL (3) of an aqueous 10 mM rhabdobranin I (1) solution, or a 10 mM 1:1 methanol: water pre-rhabdobranin B solution (3). Water or 1:1 methanol: water was applied as a control (C).

FIG. 32 shows fluorescence detection (Excitation: 488 nm, emission: 535 nm) of MyTXTL cell-free expression of eGFP over a time course of 16 hours at 37° C., in absence (red line) and presence of 34 μg/mL of rhabdobranin I (1) (green line) (Positive control: 34 μg/mL of chloramphenicol (blue line), negative control: Reaction mix without eGFP encoding plasmid (black line)).

FIG. 33 shows Rhabdobranin I (1) mediated ribosomal inhibition. A In vitro translation of Escherichia coli dihydrofolate reductase harboring a coding sequence for the Lumio labelling system in presence of 0 μM to 4000 μM rhabdobranin in triplicates. B Determination of the pixel density (PD) of the detected dihydrofolate reductase in presence of the different rhabdobranin concentrations. C The determined PD was plotted against the rhabdobranin I (1) concentration and a one-site model fit was performed in order to determine the inhibitory concentration for 50% inhibition (IC₅₀) using the GraphPad Prism 9 software (IC₅₀=99.8 μM±17.0 μM, R²=0.9728).

FIG. 34 shows identification of pre-rhabdobranin type II in Xenorhabdus doucetiae DSM 17909. A Depiction of rhabdobranin BGC ΔrdbP type II gene organization, promoter exchange location, and architecture of pCK_0402 rdbAB-L construct. B HPLC-HR-MS analysis of 0.4% L-arabinose induced and non-induced (Dashed line) strain, transformed with pCK_0402 rdbAB-L and empty vector control, with predicted prodrug structure (Structures were predicted in consideration of HR-MS data (Table 5, (Supplements)) as well as isotope labeling experiments (FIG. 57, (Supplements)).

FIG. 35 shows HPLC-HR-MS analysis of RdbP in vitro converted extract of 0.4% L-arabinose induced Xenorhabdus doucetiae DSM 17909 production strain. A Using a C18 column for the verification of type II pre-rhabdobranin derivatives, and the detection of compound 11 to 14. B Using an amide column for the analysis of the in vitro conversion with and without (Dashed line) RdbP containing membrane, and the detection of rhabdobranin type II derivative 15 (Structures were predicted in consideration of HR-MS data (Table 5, (Supplements))).

FIG. 36 shows identification of pre-rhabdobranin type III in Photorhabdus temperate HBLC135. A Depiction of rhabdobranin BGC ΔrdbP type III gene organization and promoter exchange location. B HPLC-HR-MS analysis of 0.4% L-arabinose induced and non-induced (Dashed line) strain, with predicted prodrug structure (Structures were predicted in consideration of HR-MS data (Table 5, (Supplements)) as well as isotope labeling experiments (FIG. 58, (Supplements)).

FIG. 37 shows C HPLC-HR-MS analysis of RdbP in vitro converted extract of 0.4% L-arabinose induced Photorhabdus temperate HBLC135 production strain. A Using a C18 column for the verification of pre-rhabdobranin III derivatives, and the detection of compound 11, 12 and 21. B Using an amide column for the analysis of the in vitro conversion with and without (Dashed line) RdbP containing membrane, and the detection of rhabdobranin type III derivative 22 (Structures were predicted in consideration of HR-MS data (Table 5, (Supplements)))

FIG. 38 shows an investigation of N-acetyltransferase rdbK mediated rhabdobranin self-resistance. A Conditions and mechanism of in vitro RdbK catalyzed acetylation and chemical structure of the resulting acetyl-rhabdobranin (Compound 23) (FIG. 53-56, (Supplements)). B HPLC-HR-MS analysis of the RdbK in vitro reaction mix and detection of the acetyl-rhabdobranin 1(23) (EIC: 671.456 [M+H]⁺, C₃₀H₅₉N₁₀O₇, Δppm: 0.3 (HR-MS data: Table 5, (Supplements)) in presence of acetyl CoA. C Growth curve of 0.4% L-arabinose induced Escherichia coli BL21(DE3) transformed with either pSEVA261-PBAD-rdbK in absence (green line) and presence of 0.5 mM rhabdobranin I (1) (red line), or pSEVA261-PBAD empty vector control in absence (gray line) and presence of 0.5 mM rhabdobranin I (1) (black line) (Inoculated from 0.4% L-arabinose induced pre-culture, cultured at 37° C.).

FIG. 39 shows chemical structures of pre-rhabdobranin B and pre-xenocoumacin B, with incorporated L-arginine residue highlighted in red, and pre-colibactin A, with L-alanine residue highlighted in green.

FIG. 40 shows a growth curve of Escherichia coli C41 ΔacrAB cells transformed with a pET-29b construct for the expression of Xenorhabdus budapestensis DSM 16342 RdbP (positive control) (A), XcnG (B), ClbP (C), or empty vector control (D), 1 mM IPTG induced and non-induced, in presence of 60 μg/mL pre-rhabdobranin containing fraction at 200 rpm and 37° C.

FIG. 41 shows pre-rhabdobranin modification for enhanced ClbP specificity. A Depiction of rhabdobranin BGC ΔrdbP with recombined clbB rdbF replacement, promoter exchange location, and architecture of vanillic acid inducible pAR30 clbN construct. B HPLC-MS analysis of 0.4% arabinose, 50 μM vanillic acid induced and non-induced (Dashed line) strain, detecting two engineered pre-rhabdobranin derivatives (Compound 24 and 25), with the MS data based proposed structure. C Growth curve of 1 mM IPTG induced and non-induced Escherichia coli BL21(DE3) transformed with either pAR30 rdbK or empty vector control in presence of 6 μL XAD extract containing the engineered pre-rhabdobranin derivatives.

FIG. 42 shows key ¹H-¹H COSY and HMBC correlations of rhabdobranin I (1).

FIG. 43 shows ¹H NMR (500 MHz) spectrum of rhabdobranin I (1).

FIG. 44 shows ¹³C NMR (125 MHz) spectrum of rhabdobranin I (1).

FIG. 45 shows HSQC spectrum of rhabdobranin I (1).

FIG. 46 shows ¹H-¹H COSY spectrum of rhabdobranin I (1).

FIG. 47 shows HMBC spectrum of rhabdobranin 1(1).

FIG. 48 shows ¹H NMR (500 MHz) spectrum of synthetic pre-rhabdobranin I B (3).

FIG. 49 shows ¹³C NMR (125 MHz) spectrum of synthetic pre-rhabdobranin I B (3).

FIG. 50 shows ¹H-¹H COSY spectrum of synthetic pre-rhabdobranin I B (3).

FIG. 51 shows HSQC spectrum of synthetic pre-rhabdobranin I B (3).

FIG. 52 shows HMBC spectrum of synthetic pre-rhabdobranin I B (3).

FIG. 53 shows key ¹H-¹H COSY and HMBC correlations of acetyl-rhabdobranin I (23).

FIG. 54 shows ¹H NMR (700 MHz) spectrum of acetyl-rhabdobranin I (23).

FIG. 55 shows HSQC spectrum of acetyl-rhabdobranin I (23).

FIG. 56 shows HMBC spectrum of acetyl-rhabdobranin I (23).

FIG. 57 shows an isotope labeling experiment for the verification of L-leucine incorporation in pre-rhabdobranin II derivatives. A Overview of pre-rhabdobranin II derivatives and the detected L-leucine incorporation (Red). B HR-MS analysis for the verification of L-leucine incorporation, with compound 3, 6, 7, 9, 10 showing a mass shift corresponding to the incorporation of one L-leucine and compound 5 and 8 with two leucine residues incorporated (Red spectrum: Addition of deuterated L-leucine-d10 in production culture).

FIG. 58 shows an isotope labeling experiment for the verification of L-leucine and L-valine incorporation in pre-rhabdobranin III derivatives. A Overview of pre-rhabdobranin III derivatives and the detected L-leucine (Red) and L-valine (Blue) incorporation. Notably the incorporation in 16 is based on an assumption the exact position of incorporation has to be verified by further structural analysis. The incorporation of L-valine in 17 was not considered in the structure prediction, due to the predicted sum formula. B HR-MS analysis for the verification of L-leucine and L-valine incorporation, with L-leucine identified to be incorporated in compound 16, 17 and 18, and L-valine incorporation in compound 16 and 17 (Red spectrum: Addition of deuterated L-leucine-d10, Blue Spectrum: Addition of L-valine-d8 in production culture).

FIG. 59 shows in vitro conversion and elucidated rhabdobranin I (1) structure.

FIG. 60 shows an overview of identified type I, II and III pre-rhabdobranin and rhabdobranin derivatives (Dashed boxes: Rhabdobranin derivatives predicted based on the identification of a respective prodrug molecule). The incorporation in 16 is based on an assumption the exact position of incorporation has to be verified by further structural analysis.

FIG. 61 shows a proposed mechanism of the rhabdobranin I (1) biosynthesis in Xenorhabdus budapestensis DSM 16342 and the Gcn5-Related N-Acetyltransferase mediated self-resistance mechanism (IM: Inner membrane; OM: Outer membrane) (Modified from: (Shi et al. 2022b)).

FIG. 62 shows a pre-rhabdobranin type I derivative activation by ClbP and XcnG self-resistance peptidases in relation to the RdbP catalyzed activation (IM: Inner membrane; OM: Outer membrane).

FIG. 63 shows Proposed mechanism of the modified pre-rhabdobranin I derivatives biosynthesis in an engineered Xenorhabdus budapestensis DSM 16342 ΔrdbP production strain, with the clbB C, A and PCP domain and C-terminal rdbF sequence replacing rdbF, and a clbN expression construct (Modified from: (Shi et al. 2022b)).

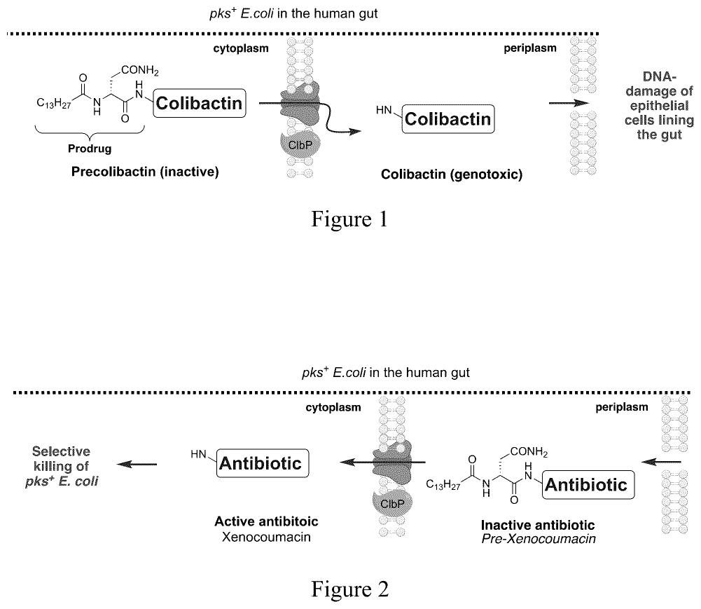

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g., 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%, 0.5% to 2.4%, 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, the terms “including,” “containing,” and “comprising” are used in their open, non-limiting sense.

As used in this disclosure, the singular forms include the plural forms and vice versa unless the context clearly indicates otherwise.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the individual.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

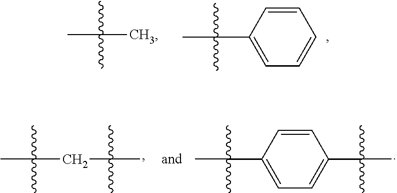

As used herein, unless otherwise indicated, the term “aliphatic group” refers to branched or unbranched hydrocarbon groups that, optionally, contain one or more degrees of unsaturation. Degrees of unsaturation include, but are not limited to, alkenyl groups, alkynyl groups, and aliphatic cyclic groups. For example, the aliphatic groups are a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C18, C₁₉, and C₂₀). The aliphatic group may be unsubstituted or substituted with one or more substituent. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Groups that are aliphatic may be alkyl groups, alkenyl groups, alkynyl groups, or carbocyclic groups, and the like.

As used herein, unless otherwise indicated, the term “alkyl” or “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₄₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C18, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, unless otherwise indicated, the term “aryl” or “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C18, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, and the like.

As used herein, the term “heteroaryl” or “hereteroaryl” refers to a monocyclic or bicyclic ring system comprising one or two aromatic rings and containing at least one nitrogen or oxygen atom in an aromatic ring. Unless otherwise indicated, a heteroaryl group can be unsubstituted or substituted with one or more, and in particular one or two, substituents. Non-limiting examples of substituents include halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Examples of heteroaryl groups include, benzofuranyl, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups, and substituents analogs of any of the foregoing heteroaryl groups.

As used herein, unless otherwise indicated, halogen means fluorine, chlorine, bromine, and iodine, and halo means fluoro, chloro, bromo, and iodo.

Amino acids and amino acid residues may be referred to herein by either their commonly known three letter symbols or by the one-letter symbols recommended by the IUPAC-IUB Biochemical Nomenclature Commission.

The present disclosure provides methods of preventing colorectal cancer and/or killing colibactin-producing E. coli. Also provided are prodrug antibiotics suitable for preventing colorectal cancer and/or killing colibactin-producing E. coli, and methods of making prodrug antibiotics.

In an aspect, the present disclosure provides various methods. The methods may be methods for killing E. coli that product colibactin (ClbP) and/or preventing colorectal cancer in a subject and/or treating colorectal cancer. The methods may also be suitable to prevent or treat other conditions associated with ClbP producing E. coli. In various examples, the method may kill any bacteria that expresses ClbP protease.

A method may be for killing a least a portion of an E. coli population, where at least some or all the E. coli expresses ClbP. The method may comprise contacting at least a portion of the E. coli with a prodrug antibiotic at a therapeutically effective amount of the resulting drug. Following contact with the E. coli, the prodrug is cleaved to form a drug, resulting in the death of at least some of the ClbP-expressing E. coli.

In various examples, the prodrug antibiotics of the present disclosure selectively kill strains of Clb⁺E. coli, while not affecting other strains of E. coli present. In various embodiments, a method of the present disclosure may also prevent in cancer initiation of colorectal cancer in subjects having colitis, irritable bowel syndrome, or the like. Similarly, without intending to be bound by any particular theory, it is considered that the method may also be used in treating other conditions and diseases associated with Clb⁺E. coli.

Various prodrug antibiotics may be used. For example, a prodrug antibiotic may be

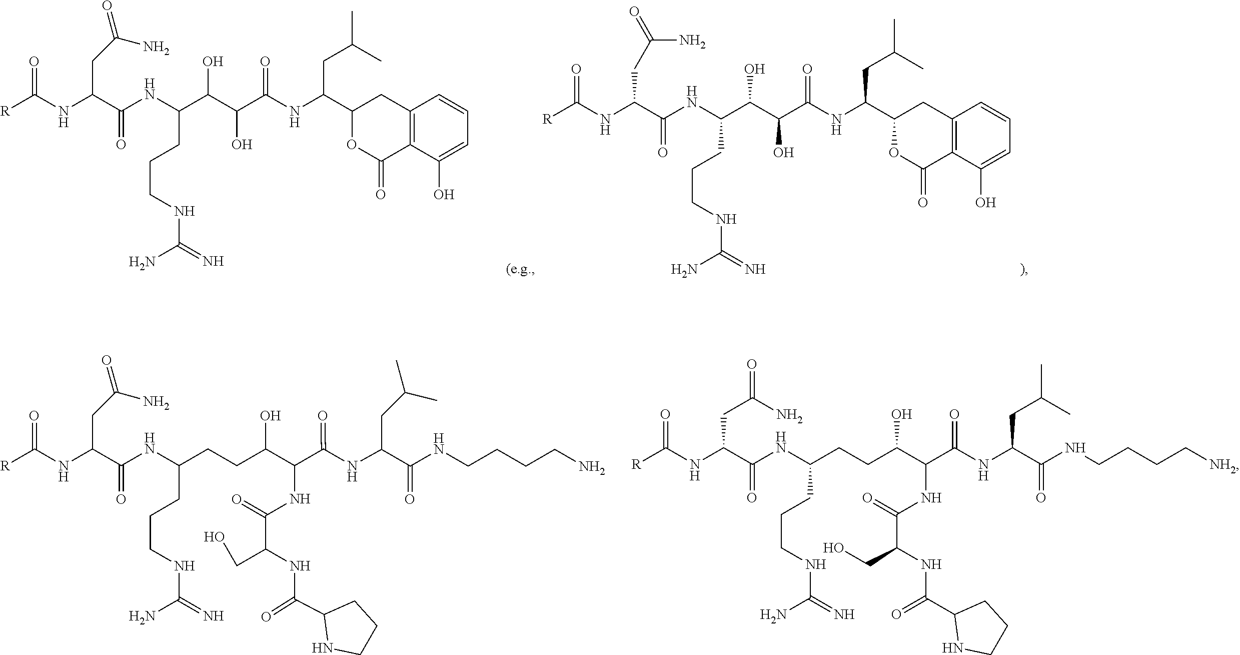

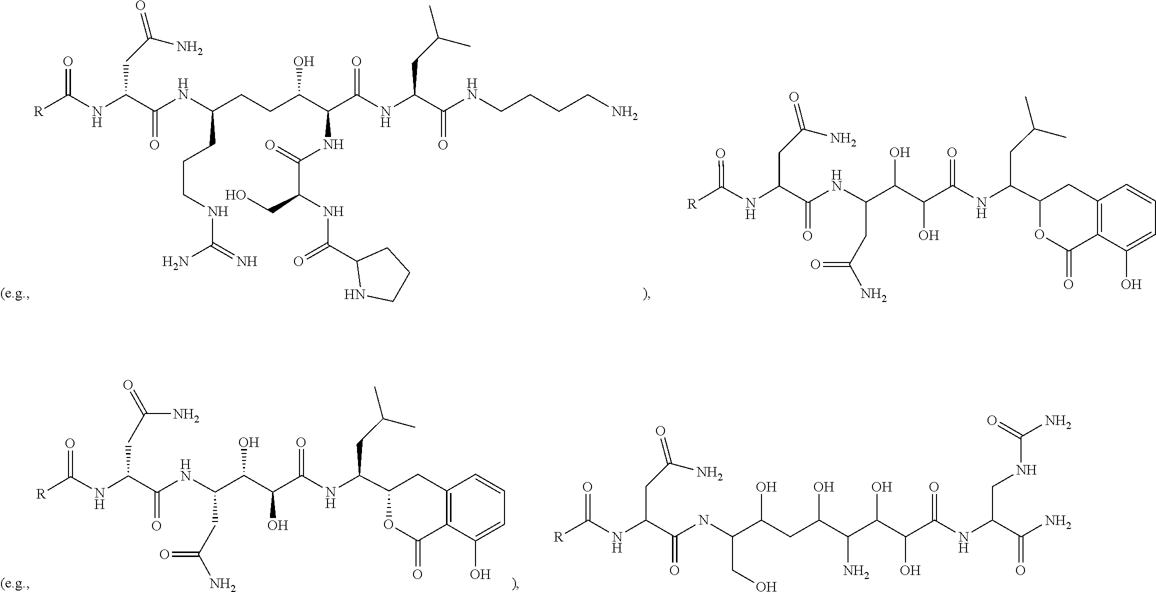

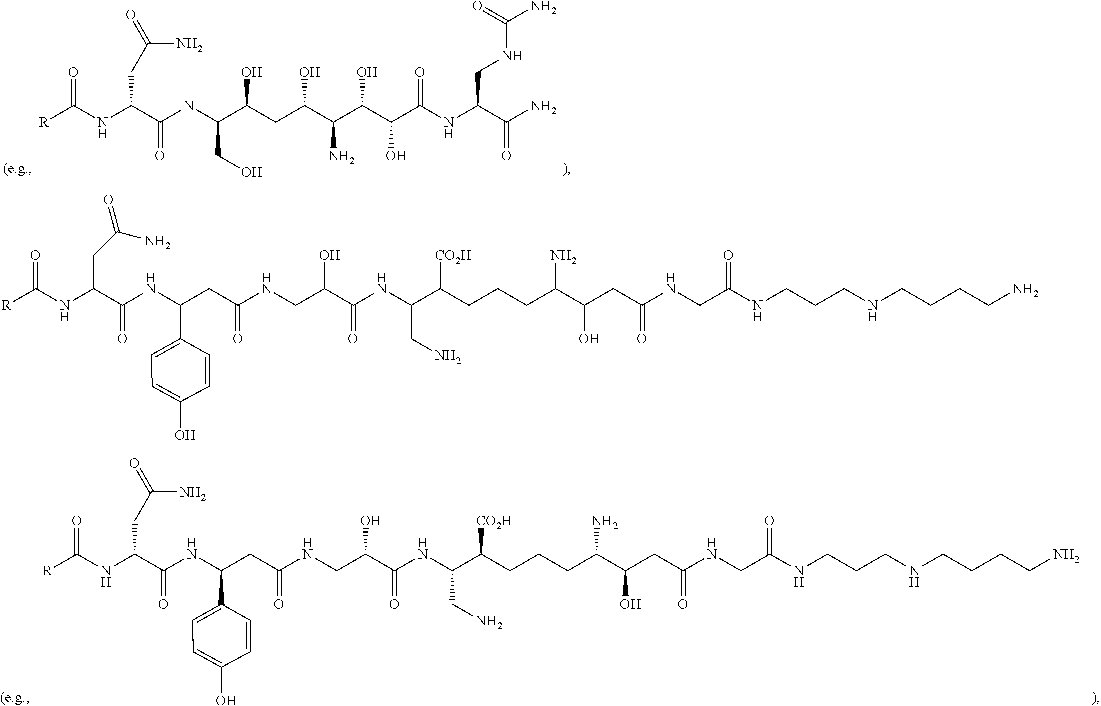

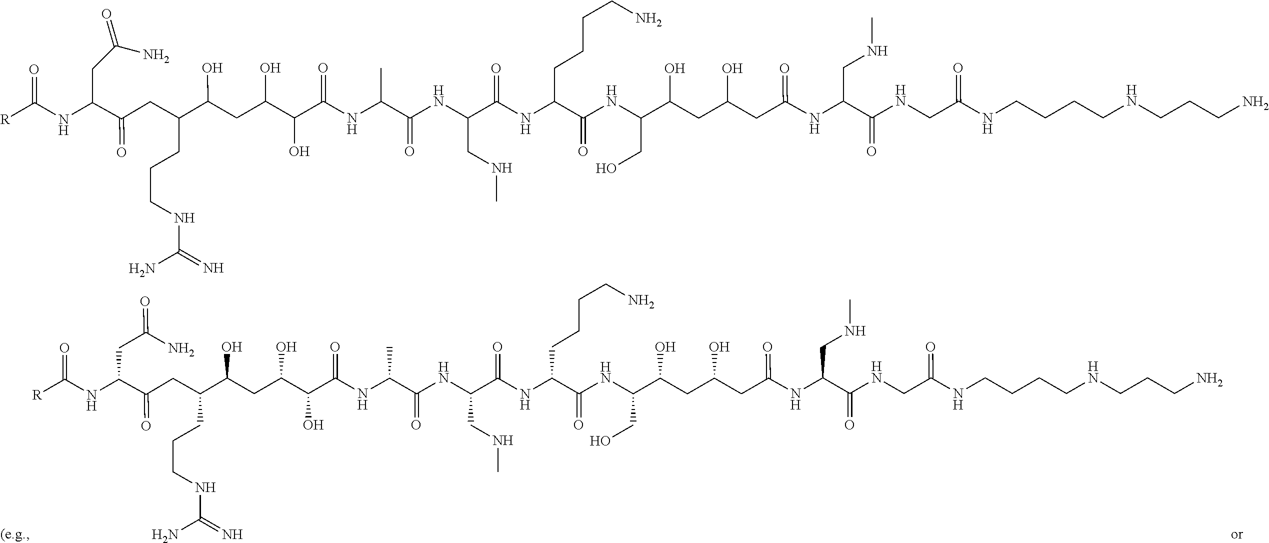

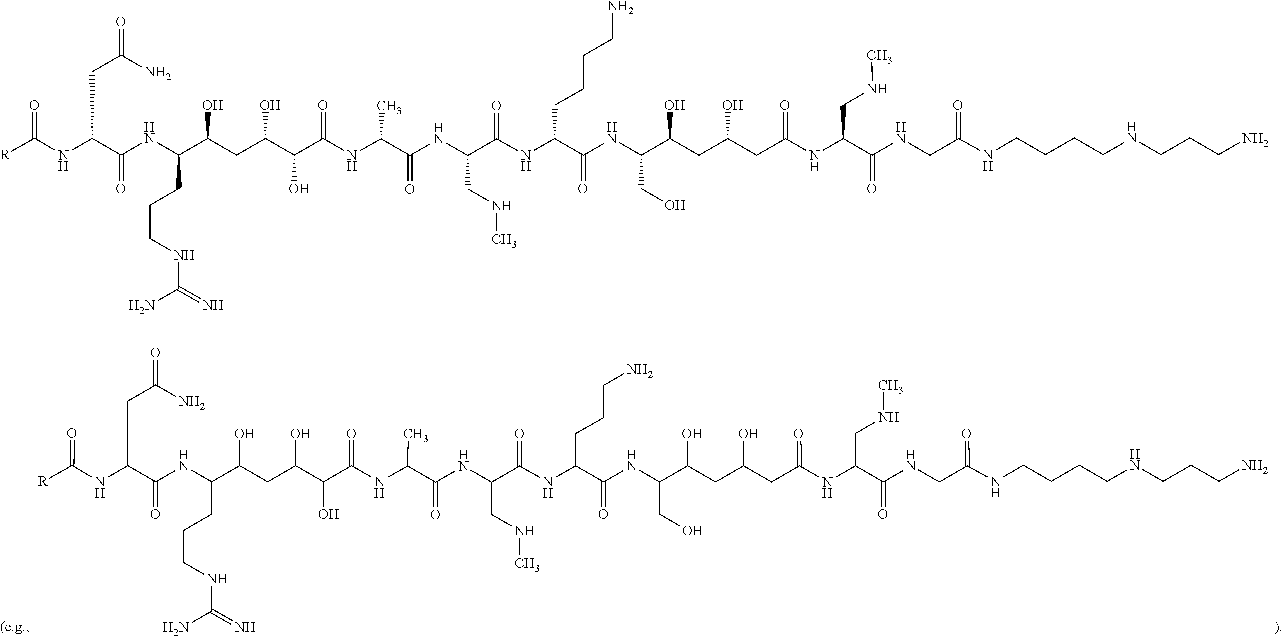

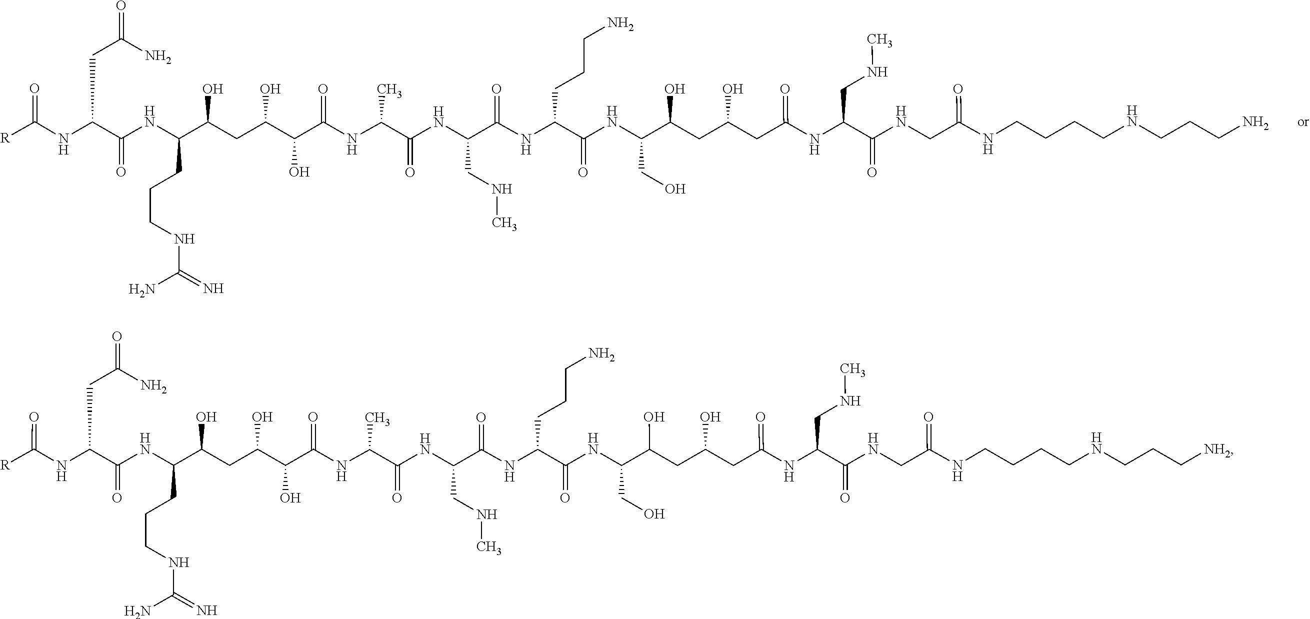

or any combination thereof, wherein R is a substituted or unsubstituted aliphatic group. In various examples, the prodrug antibiotic may be a salt or in the form of a composition.

Various R groups may be used. For example, the R group may be an aliphatic group 1 to 25 carbons atoms in length (e.g., 1 to 22 carbon atoms in length, 1 to 18 carbon atoms in length, or 7 to 25 carbon atoms in length) (e.g., 1, 2, 3, 4, 5, 6, 7, 8, 9, 10, 11, 12, 13, 14, 15, 16, 17, 18, 19, 20, 21, 22, 23, 24, or 25 carbon atoms in length). The aliphatic group may be substituted or unsubstituted. The aliphatic group may have one or more aromatic rings (e.g., aryl or heteroaryl groups), one or more pi bonds (e.g., one or more double bonds and/or one or more triple bonds), one or more hydroxy groups, and/or one or more halogens (e.g., fluoro groups). The one or more pi bonds may be conjugated. For example, aromatic rings may be pendent substituents, terminal substituents, or the aromatic rings may interrupt a linear aliphatic chain of the aliphatic group such that a portion of the aliphatic group is one substituent on the aromatic ring and the remaining portion of the aliphatic chain is a different substituent on the aromatic ring

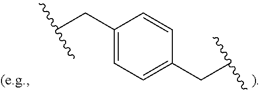 [SMILES: *Cc1ccc(C*)cc1]

The aromatic rings may have one or more additional substituents. For example, R may have the following structure:

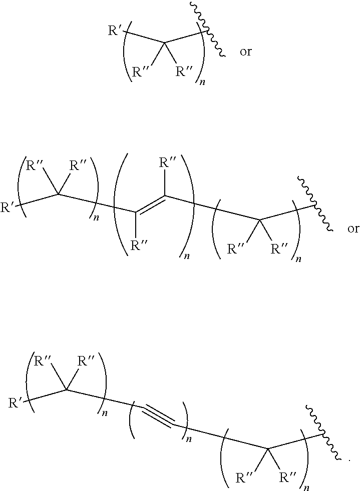

R′ is hydrogen, a halogen (e.g., bromo, fluoro, or the like), an aryl group (e.g., phenyl, napthyl, or the like), a methyl group, halogenated methyl group (e.g., —CF₃), an amino group, or a hydroxy, or the like. An R′ group may be unsubstituted or further substituted. R″ is hydrogen, a halogen (e.g., bromo, fluoro, or the like), an aryl group (e.g., phenyl, napthyl, or the like), a methyl group, halogenated methyl group (e.g., —CF₃), a hydroxy, or the like. Each R″ may be the same or different. As an illustrative example, on a carbon atom substituted with two R″ groups, one R″ is hydrogen, and one R″ is methyl. In another illustrative example, if R is

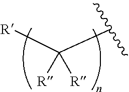

and n is 2, each of the four resulting R″ groups may be the same or different. For example, one R″ is a methyl, one R″ is hydrogen, one R″ is fluoro, and the final R″ is bromo. Other combinations of substituents on these genuses are contemplated by this disclosure. Each n is independently 0 to 18 (e.g., 0, 1, 2, 3, 4, 5, 6, 7, 8, 9, 10, 11, 12, 13, 14, 15, 16, 17, 18). In various examples, R may have the following structure:

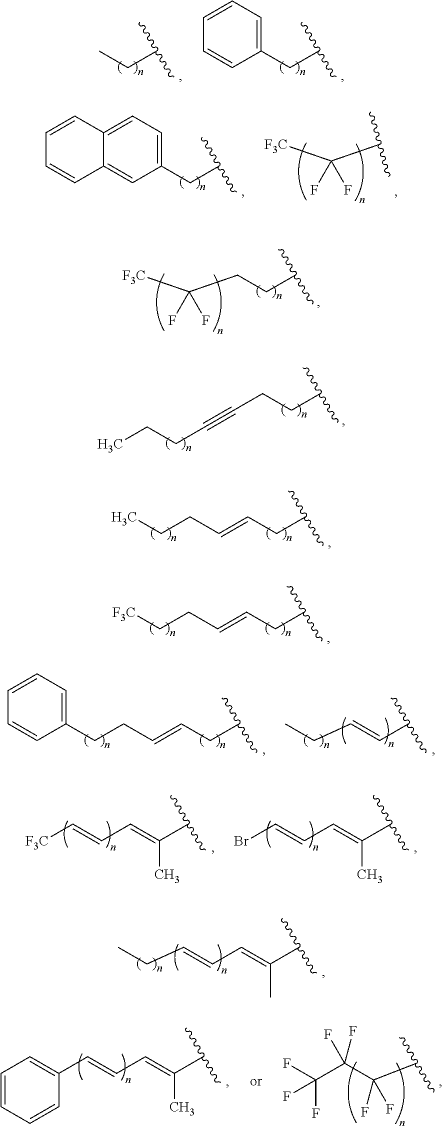

wherein each n is independently 0 to 18 (e.g., 0, 1, 2, 3, 4, 5, 6, 7, 8, 9, 10, 11, 12, 13, 14, 15, 16, 17, 18). Additional examples of R groups include, but are not limited to:

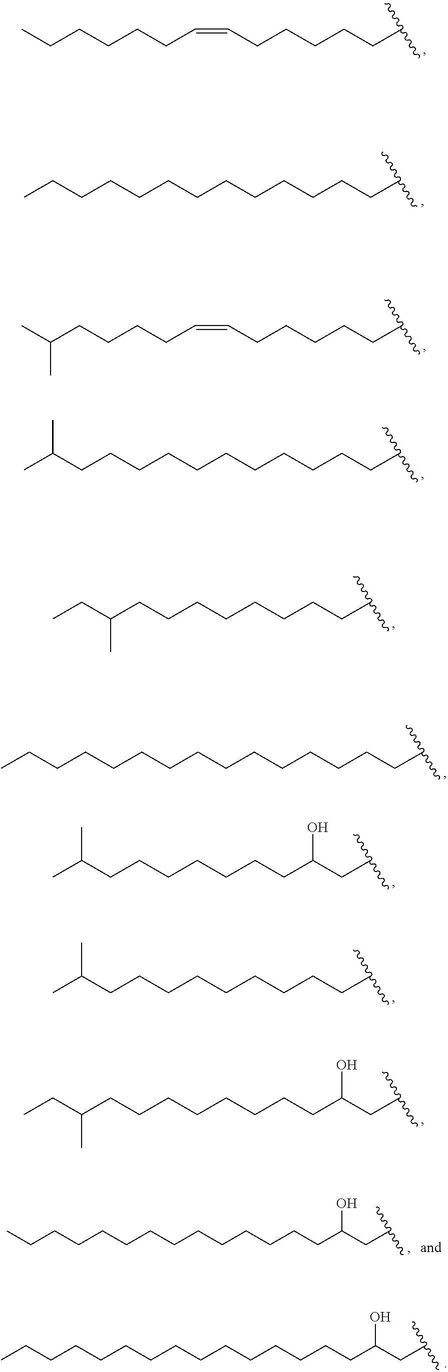

In various examples, the prodrug antibiotic has the following structure:

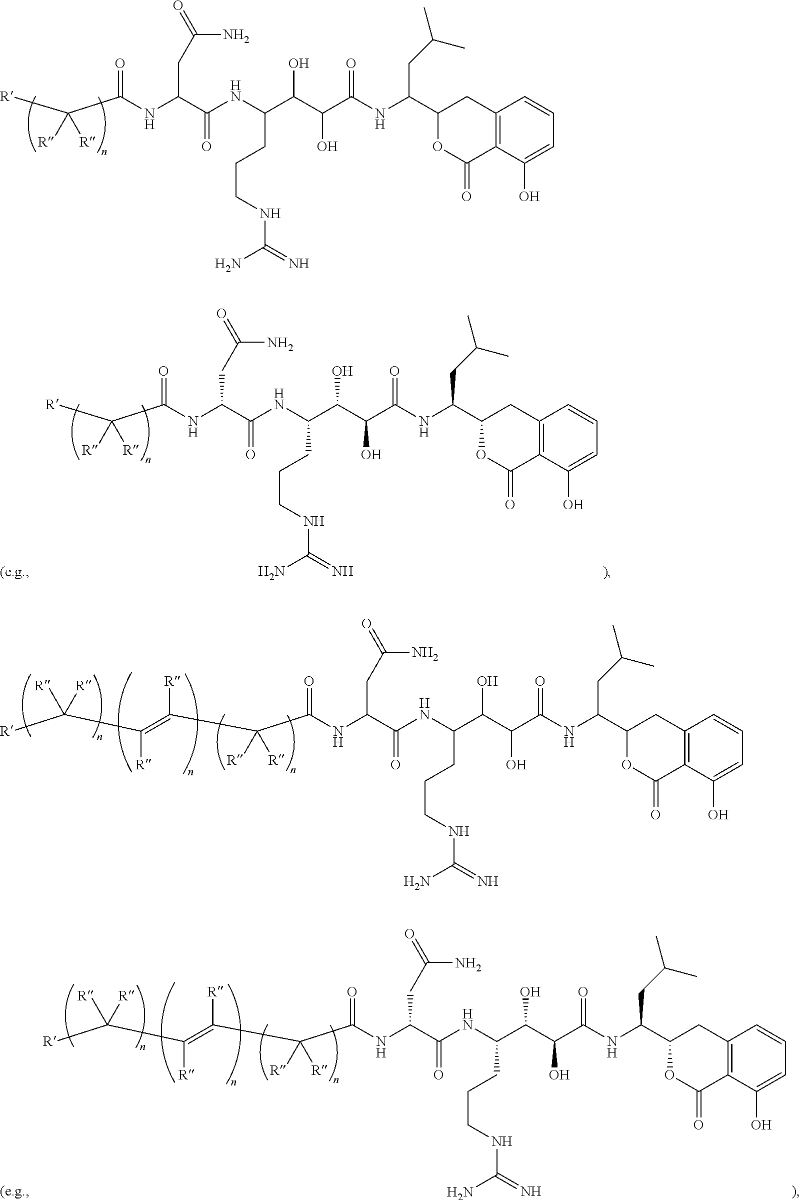

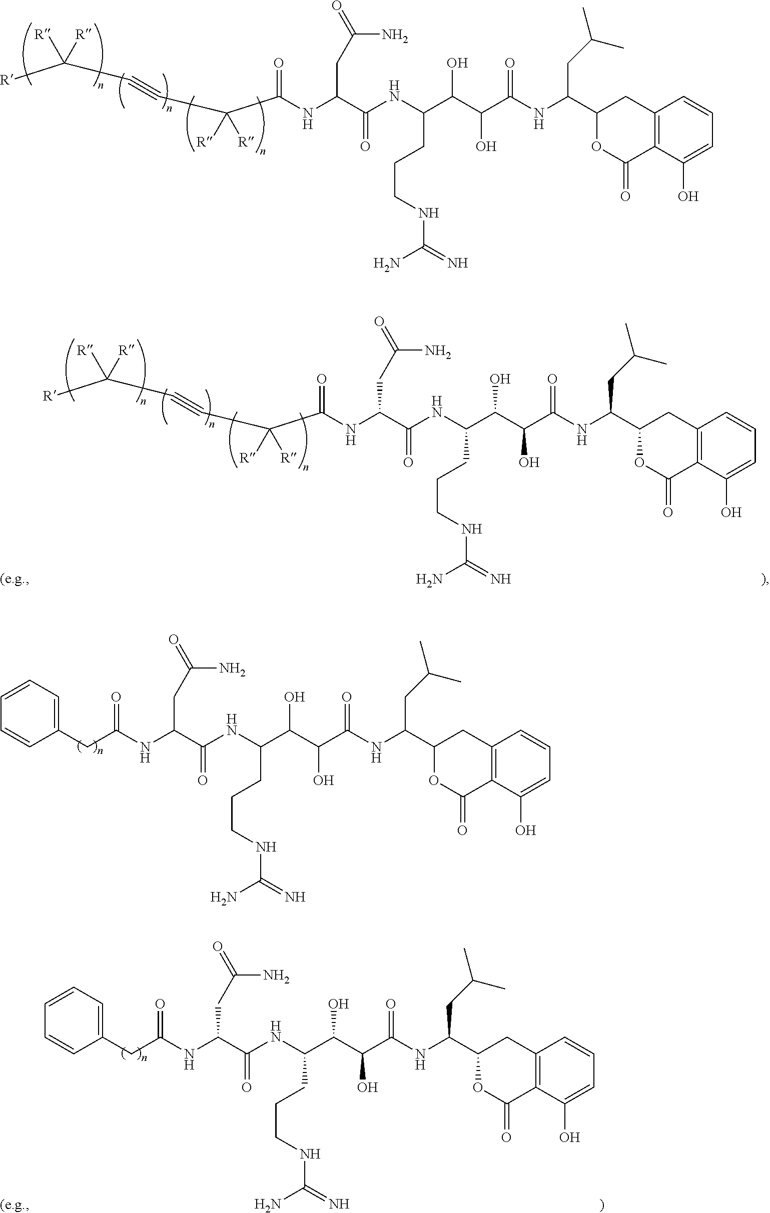

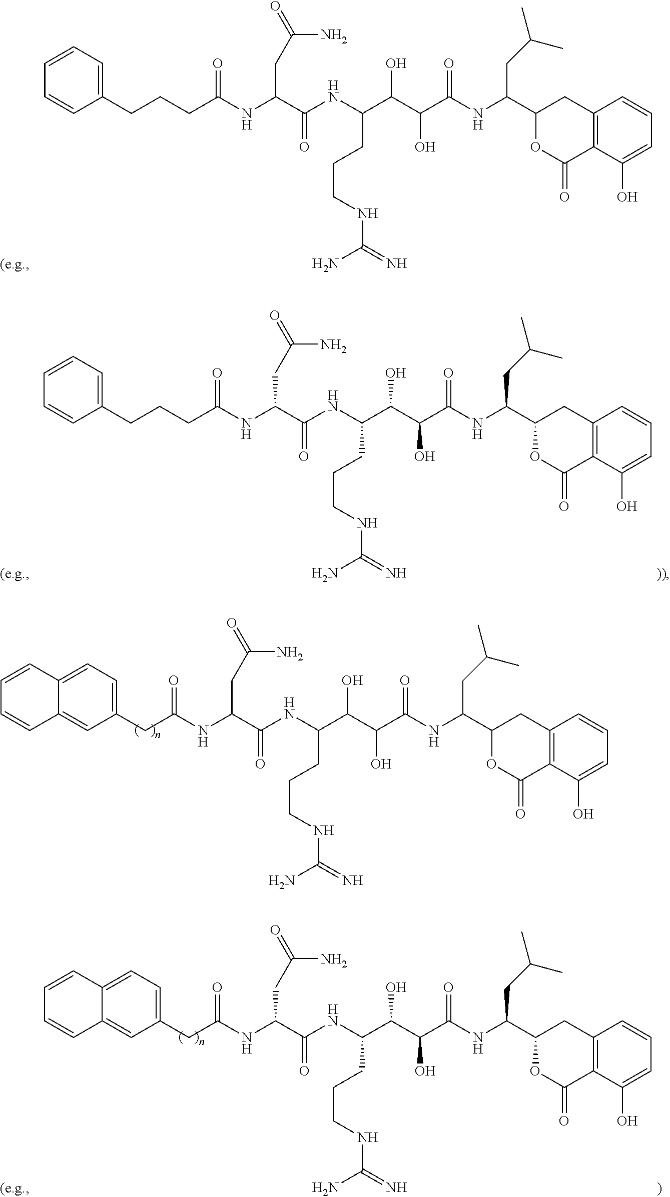

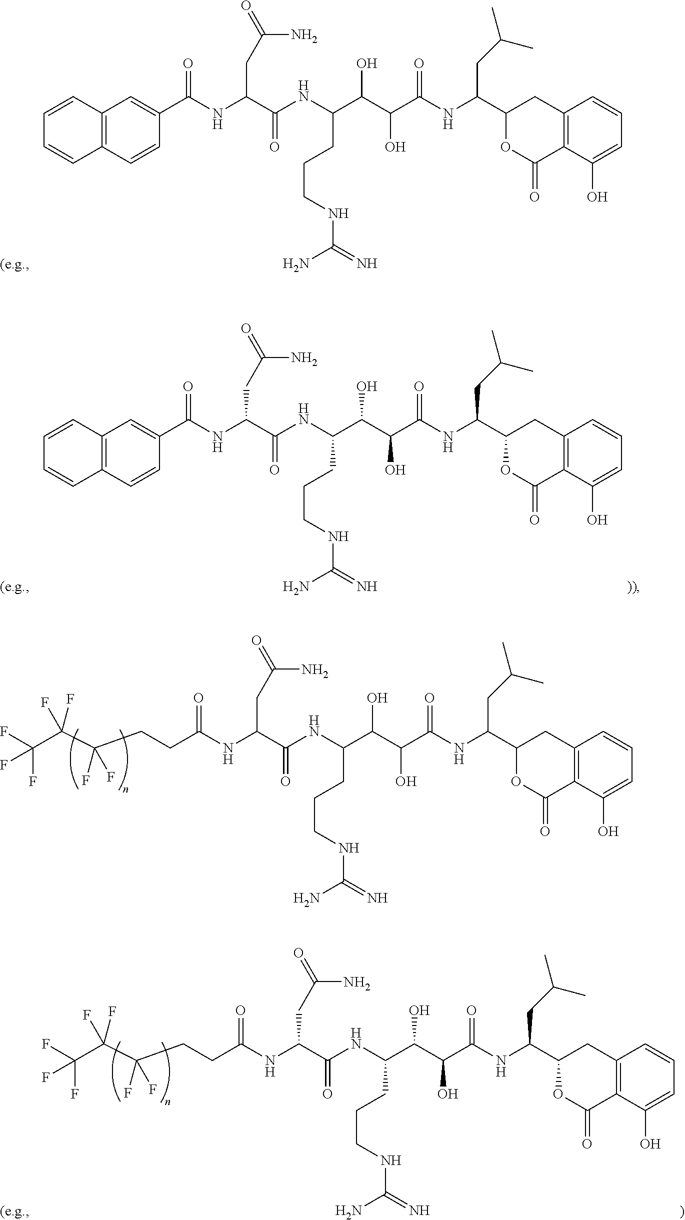

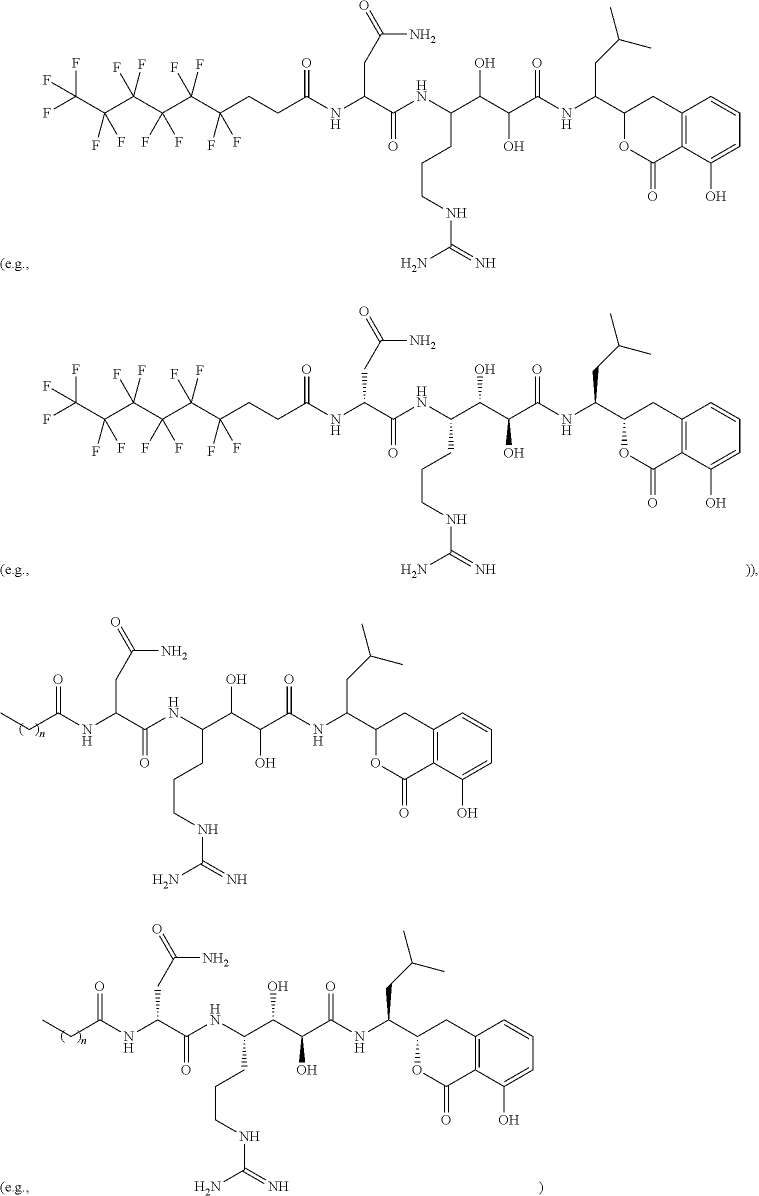

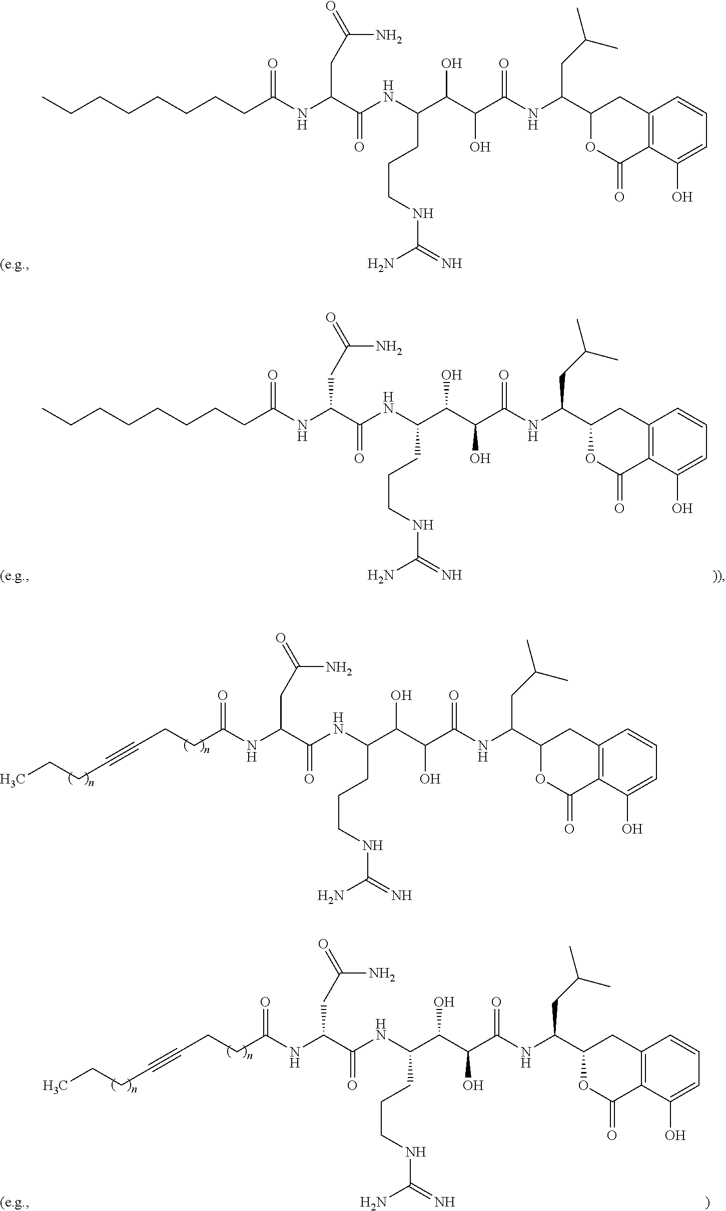

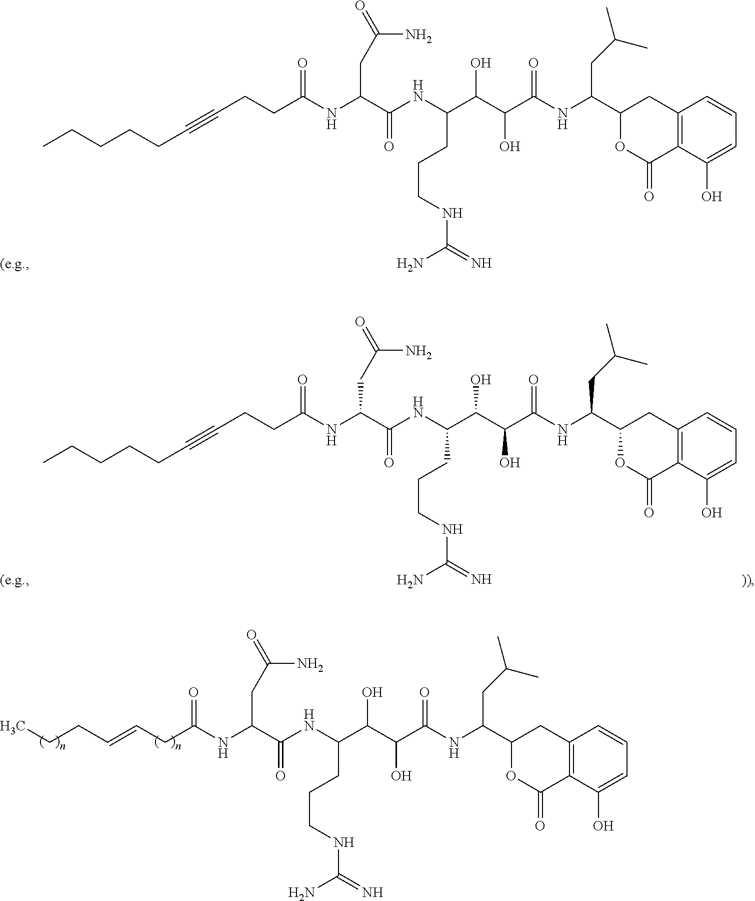

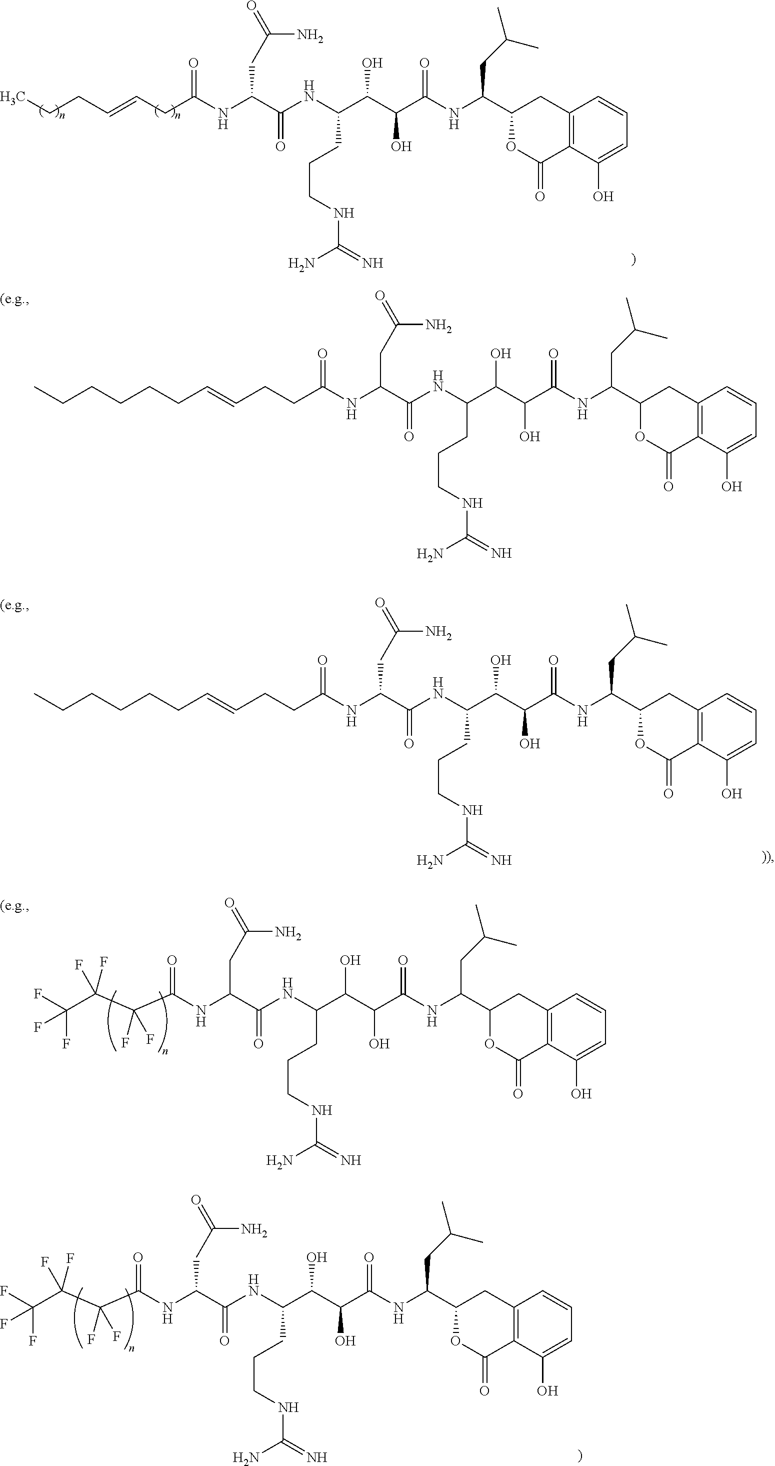

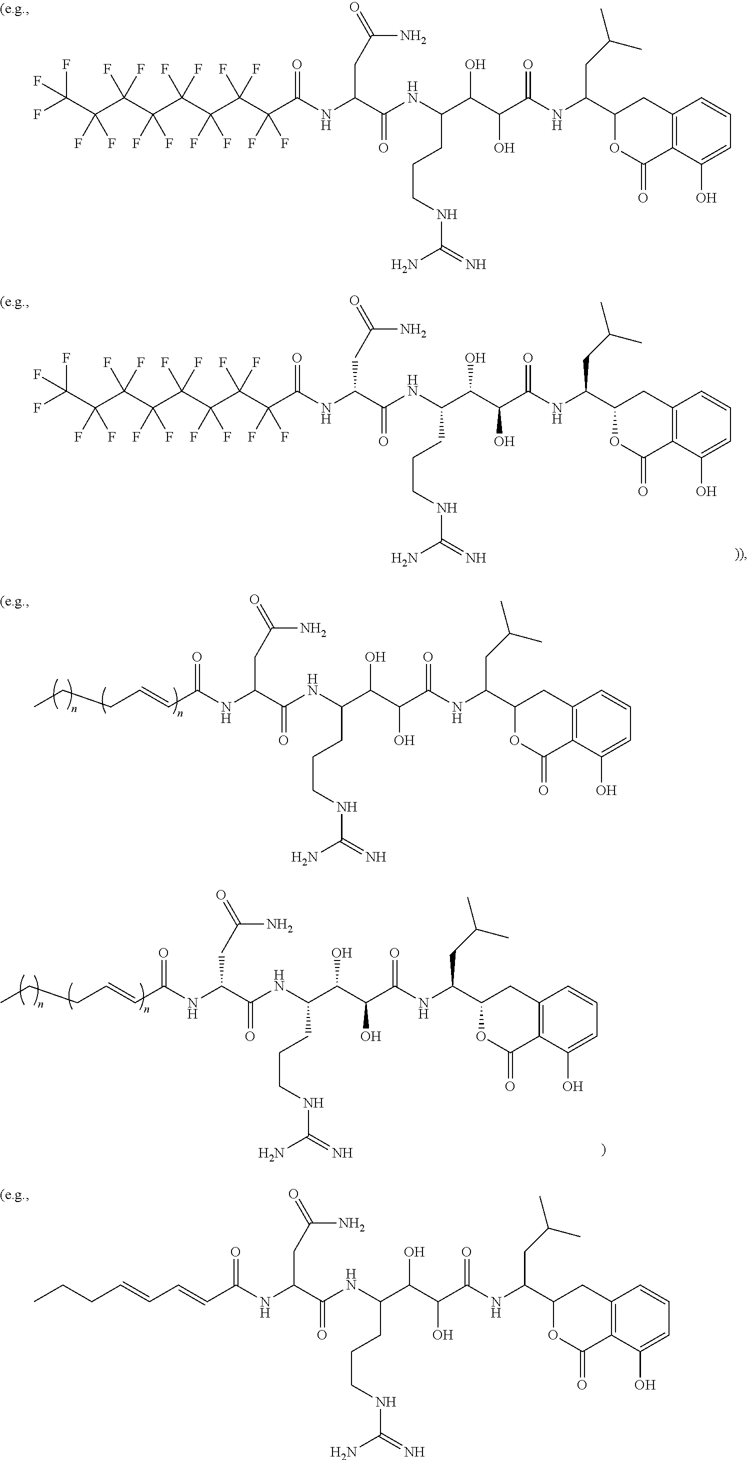

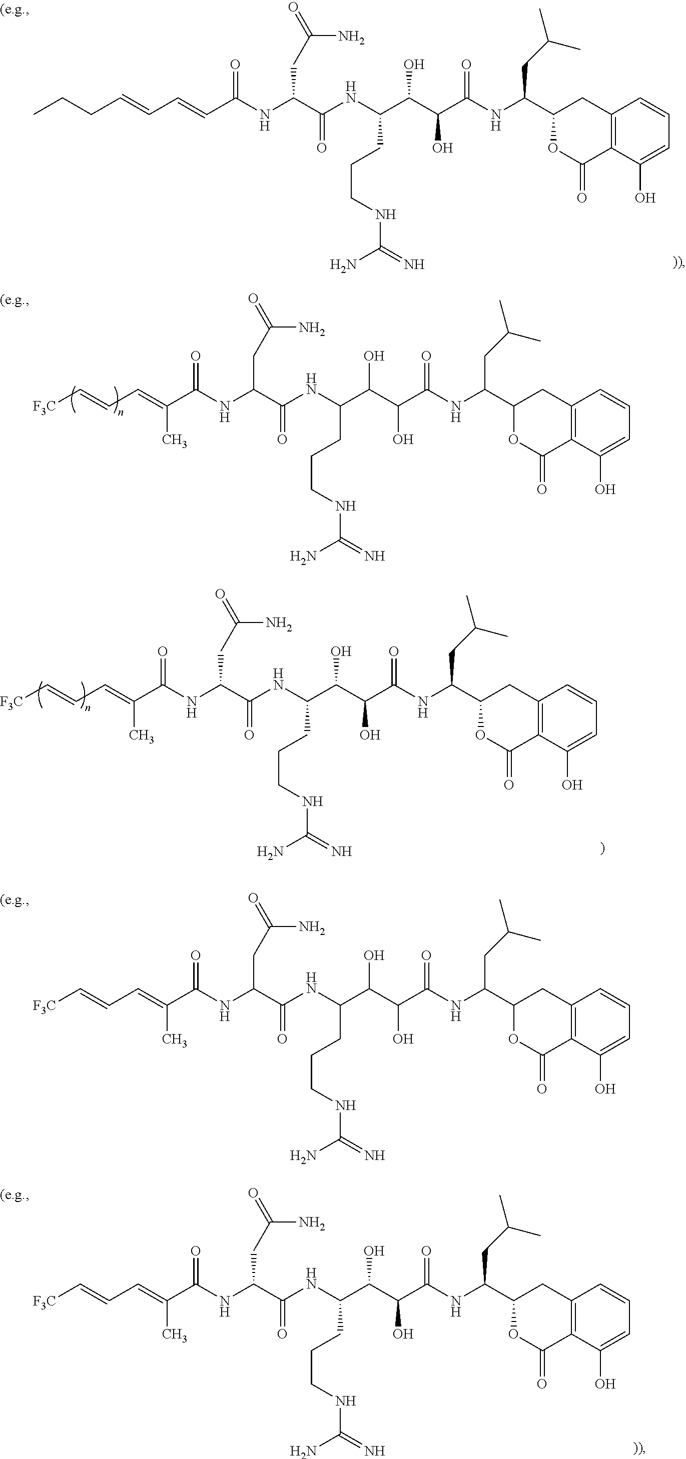

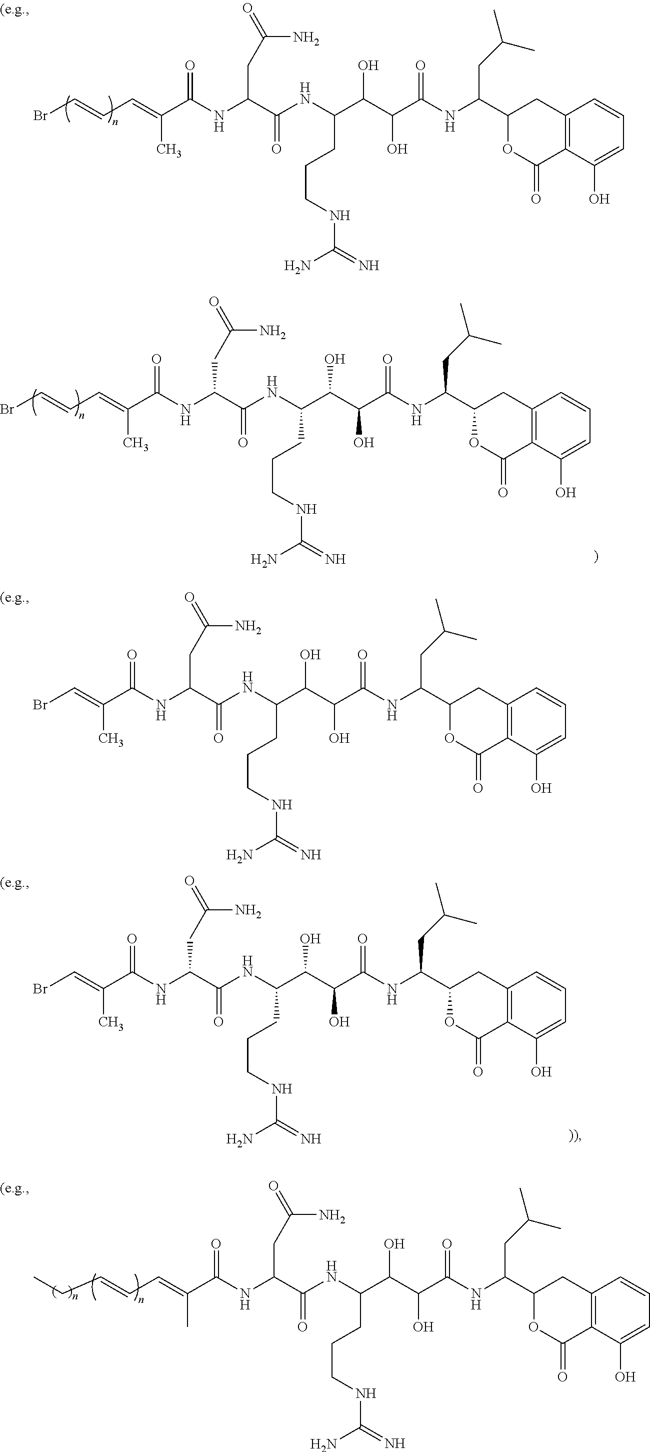

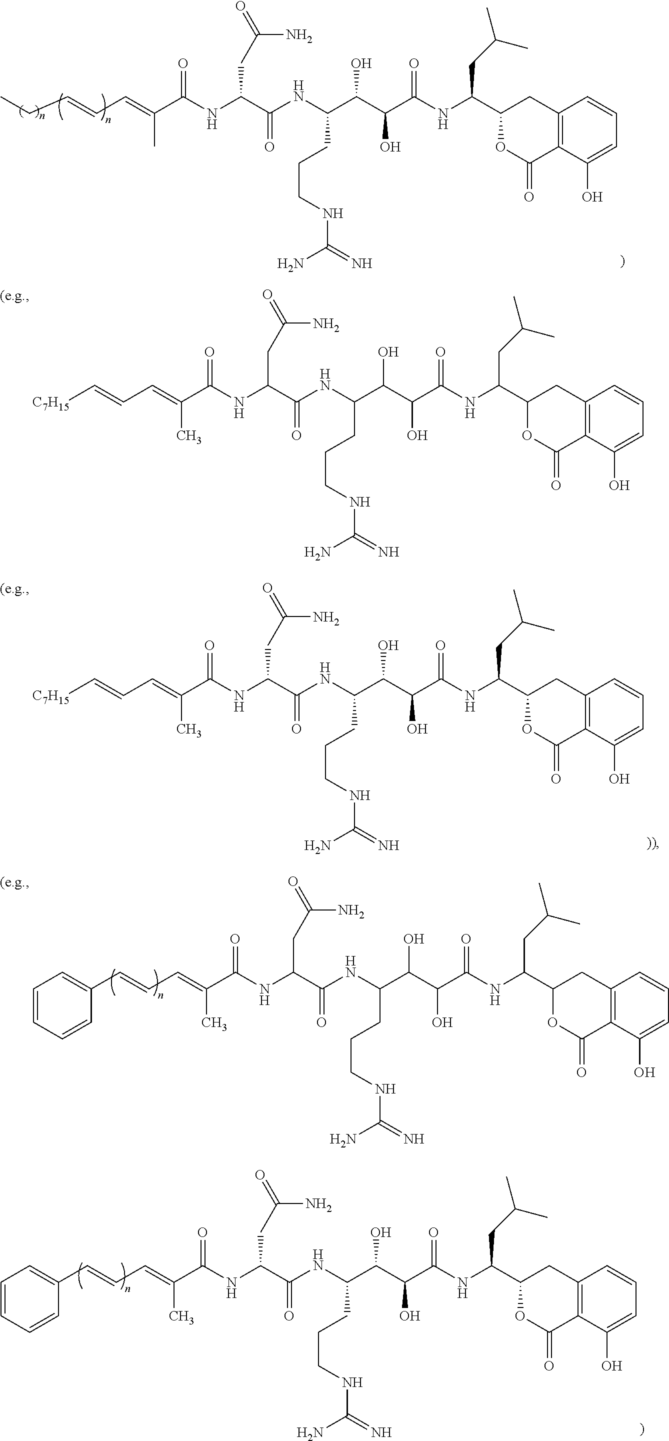

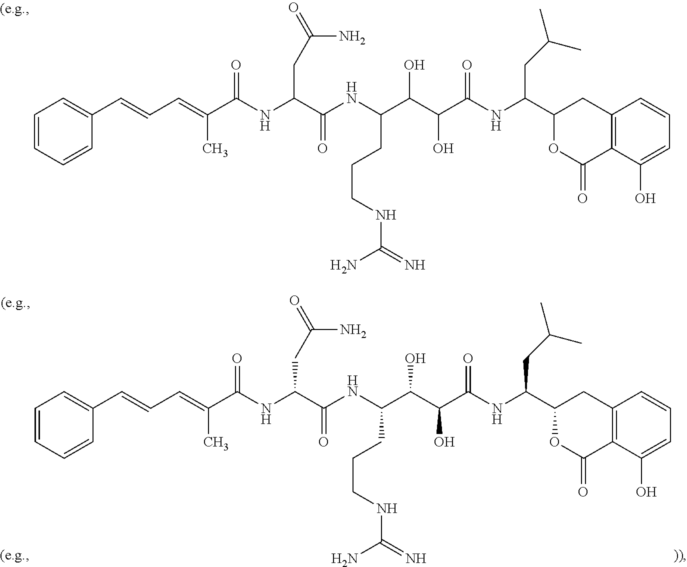 [SMILES: C/C(=C\C=C\c1ccccc1)C(=O)NC(CC(N)=O)C(=O)NC(CCCNC(=N)N)C(O)C(O)C(=O)NC(CC(C)C)C1Cc2cccc(O)c2C(=O)O1 ; C/C(=C\C=C\c1ccccc1)C(=O)N[C@H](CC(N)=O)C(=O)N[C@@H](CCCNC(=N)N)[C@H](O)[C@H](O)C(=O)N[C@@H](CC(C)C)[C@@H]1Cc2cccc(O)c2C(=O)O1]

or any combination thereof, wherein each n is independently 0 to 18.

In various other examples, the prodrug antibiotic has the following structure:

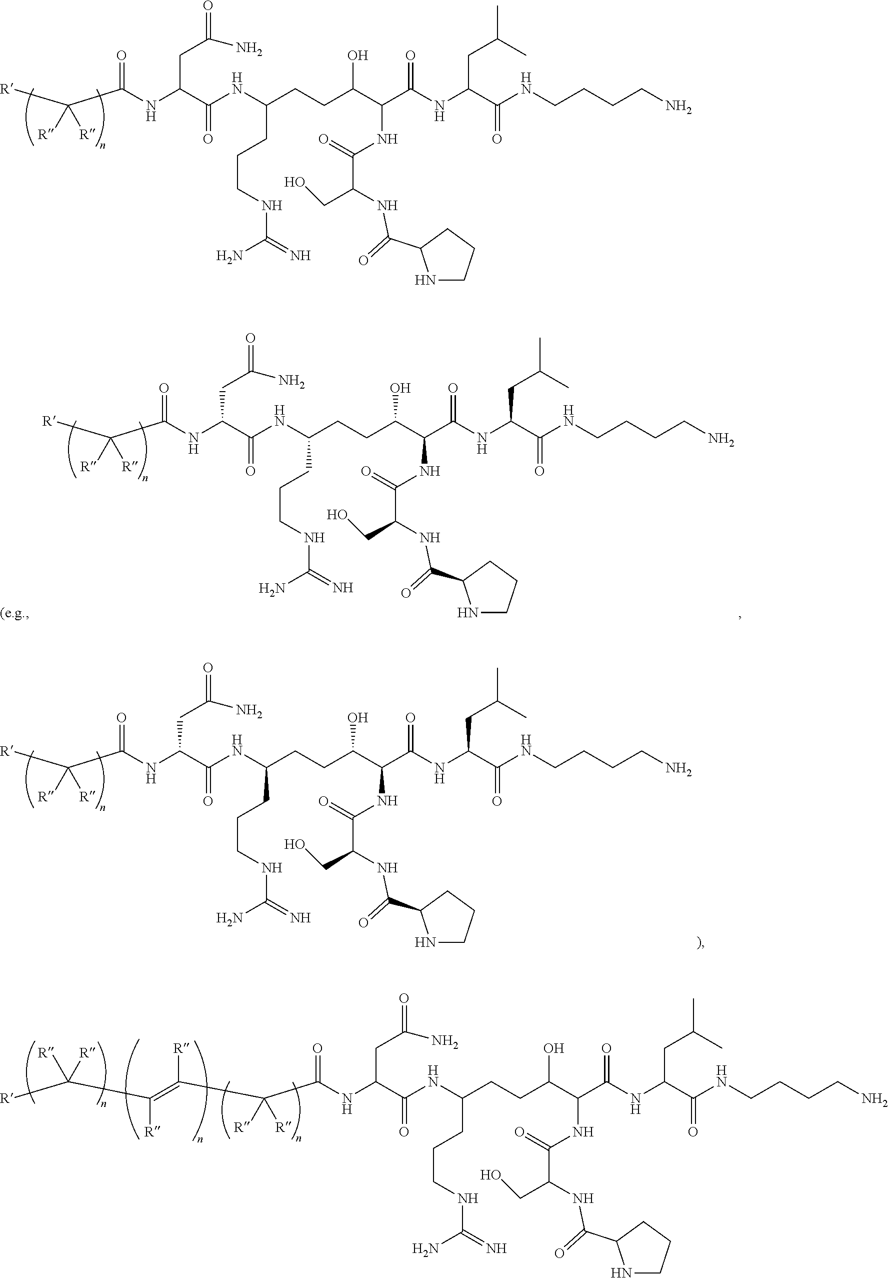

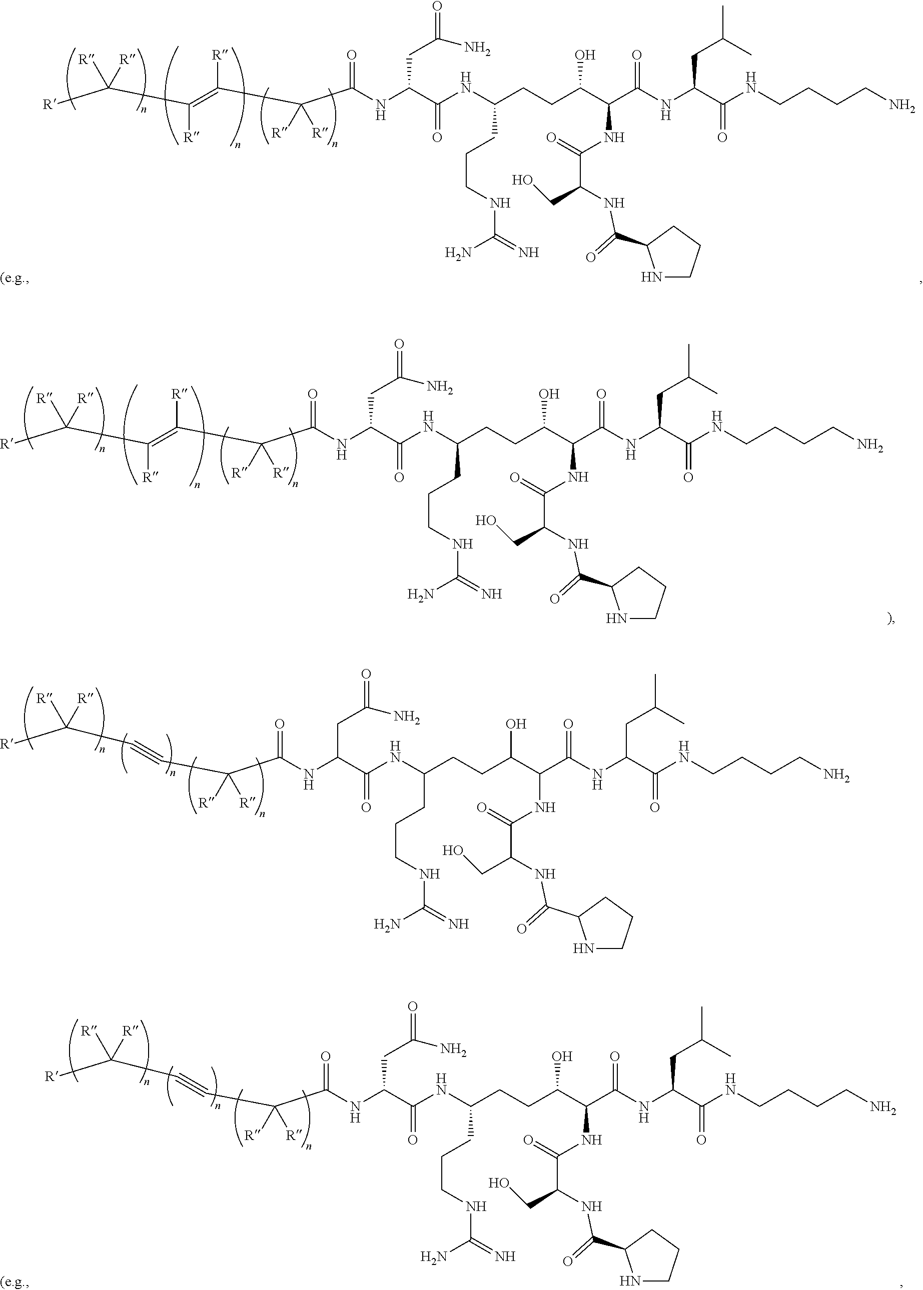

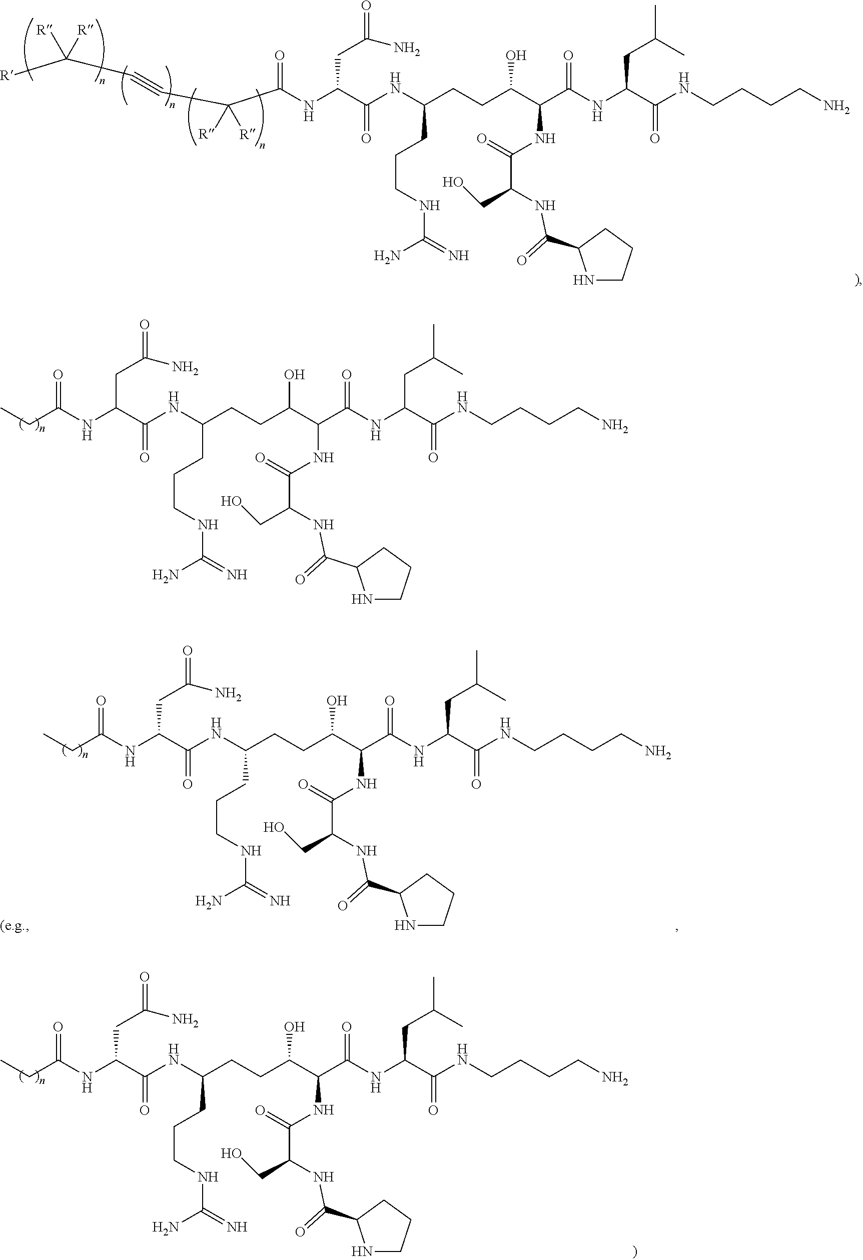

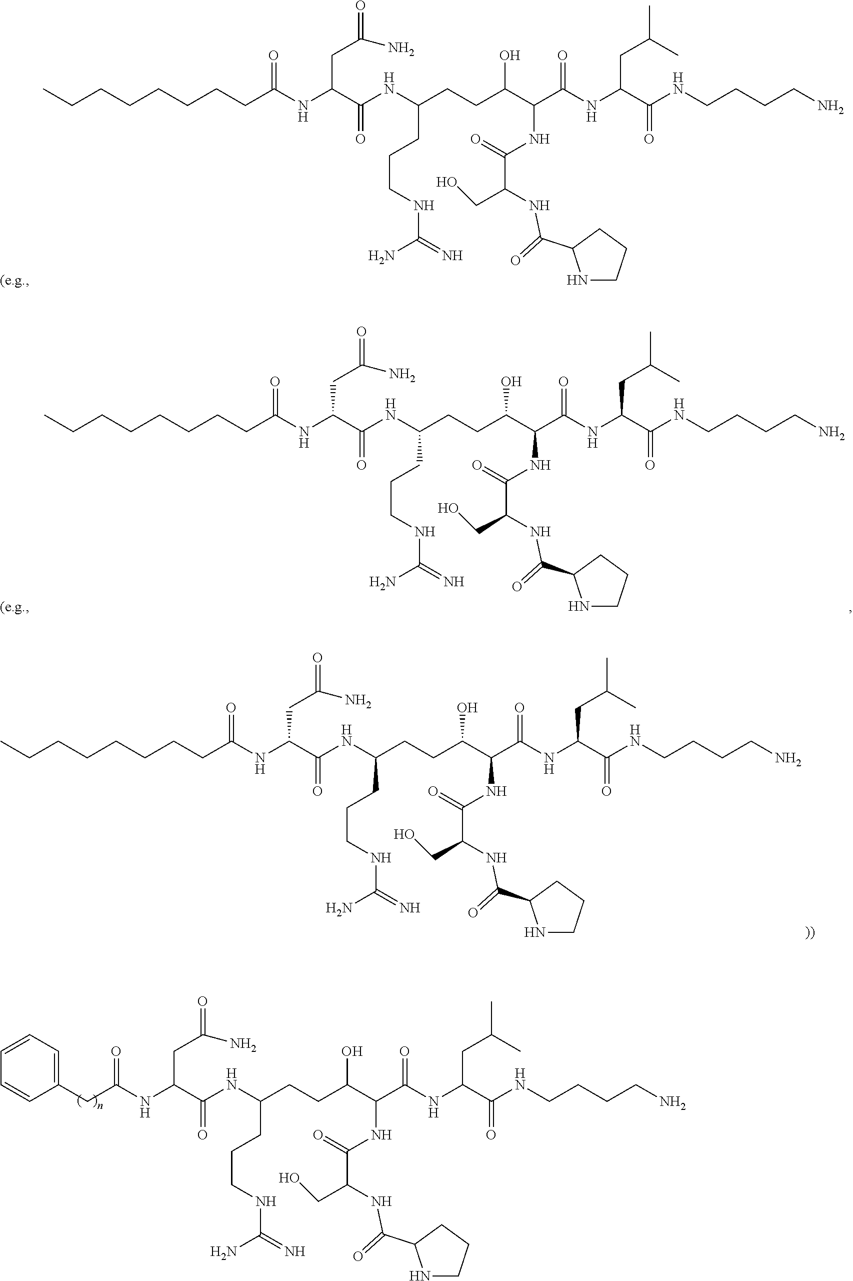

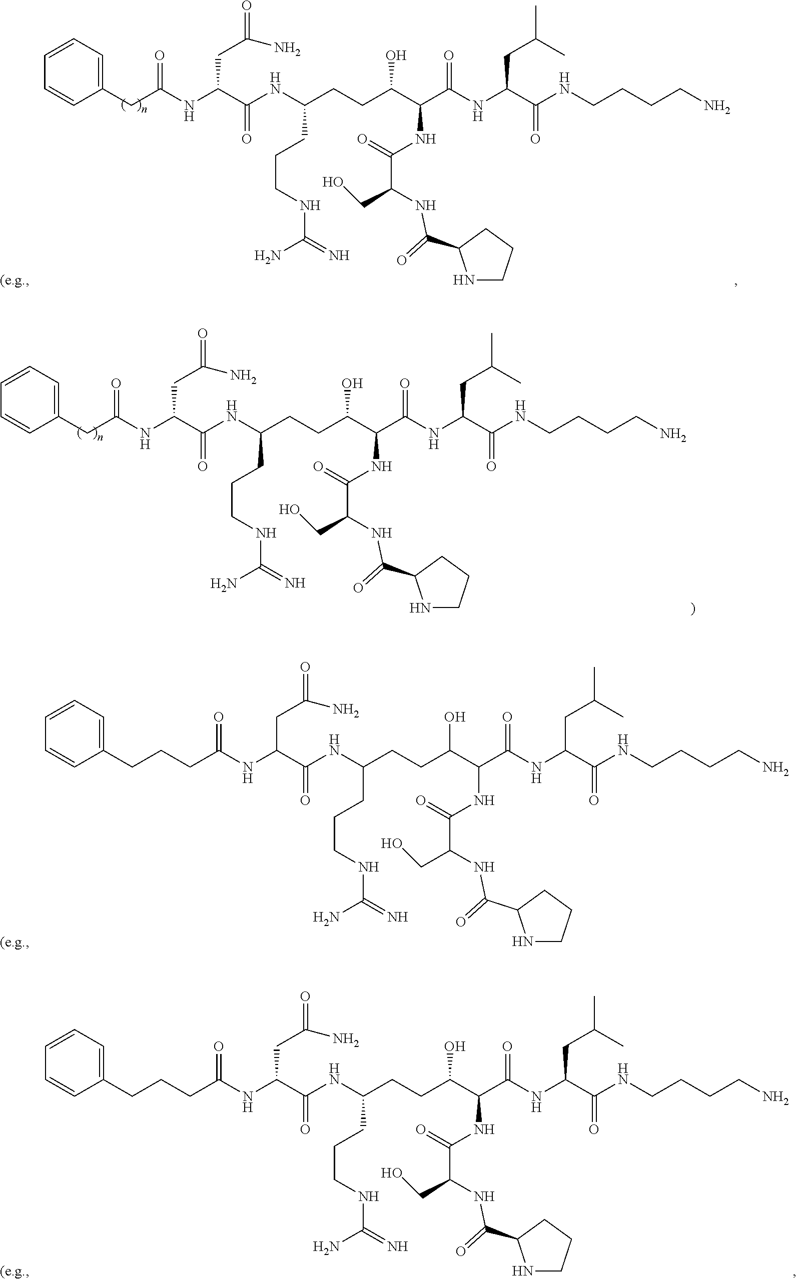

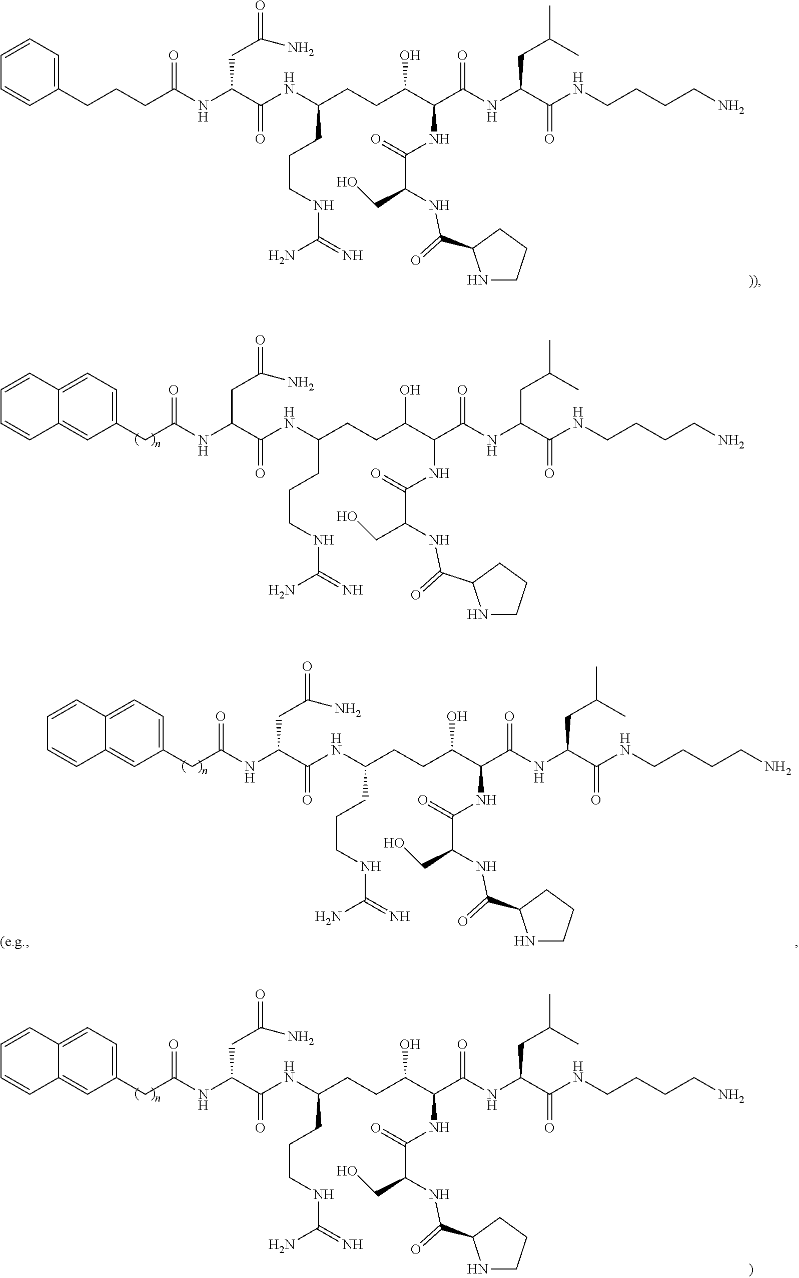

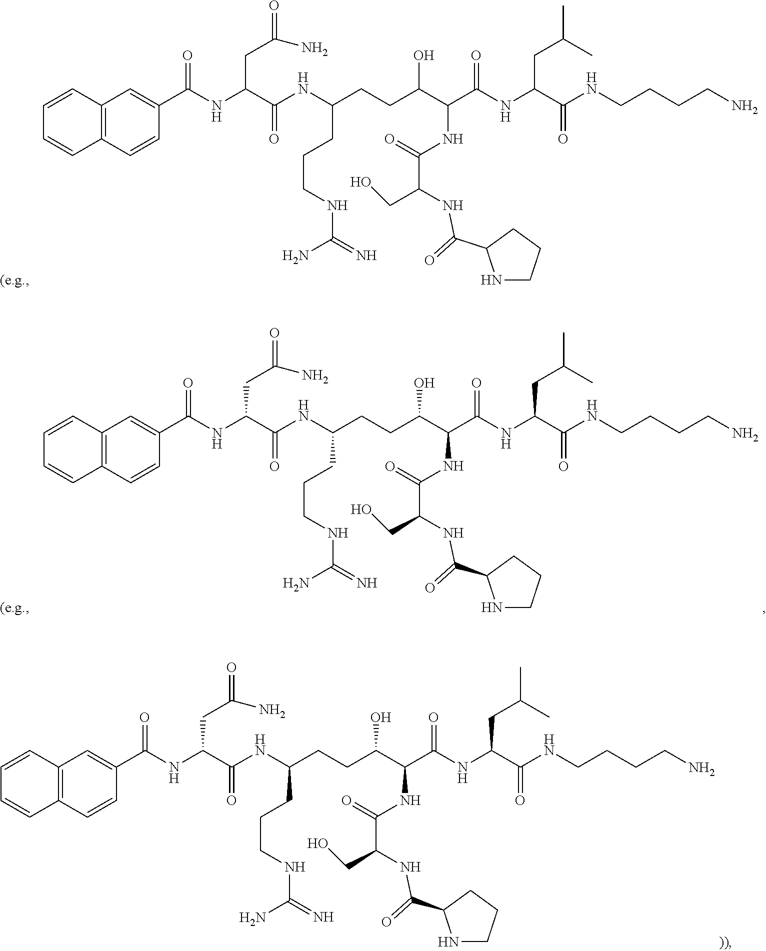 [SMILES: CC(C)CC(NC(=O)C(NC(=O)C(CO)NC(=O)C1CCCN1)C(O)CCC(CCCNC(=N)N)NC(=O)C(CC(N)=O)NC(=O)c1ccc2ccccc2c1)C(=O)NCCCCN ; CC(C)C[C@H](NC(=O)[C@@H](NC(=O)[C@H](CO)NC(=O)[C@H]1CCCN1)[C@@H](O)CC[C@H](CCCNC(=N)N)NC(=O)[C@@H](CC(N)=O)NC(=O)c1ccc2ccccc2c1)C(=O)NCCCCN ; CC(C)C[C@H](NC(=O)[C@@H](NC(=O)[C@H](CO)NC(=O)[C@H]1CCCN1)[C@@H](O)CC[C@@H](CCCNC(=N)N)NC(=O)[C@@H](CC(N)=O)NC(=O)c1ccc2ccccc2c1)C(=O)NCCCCN]

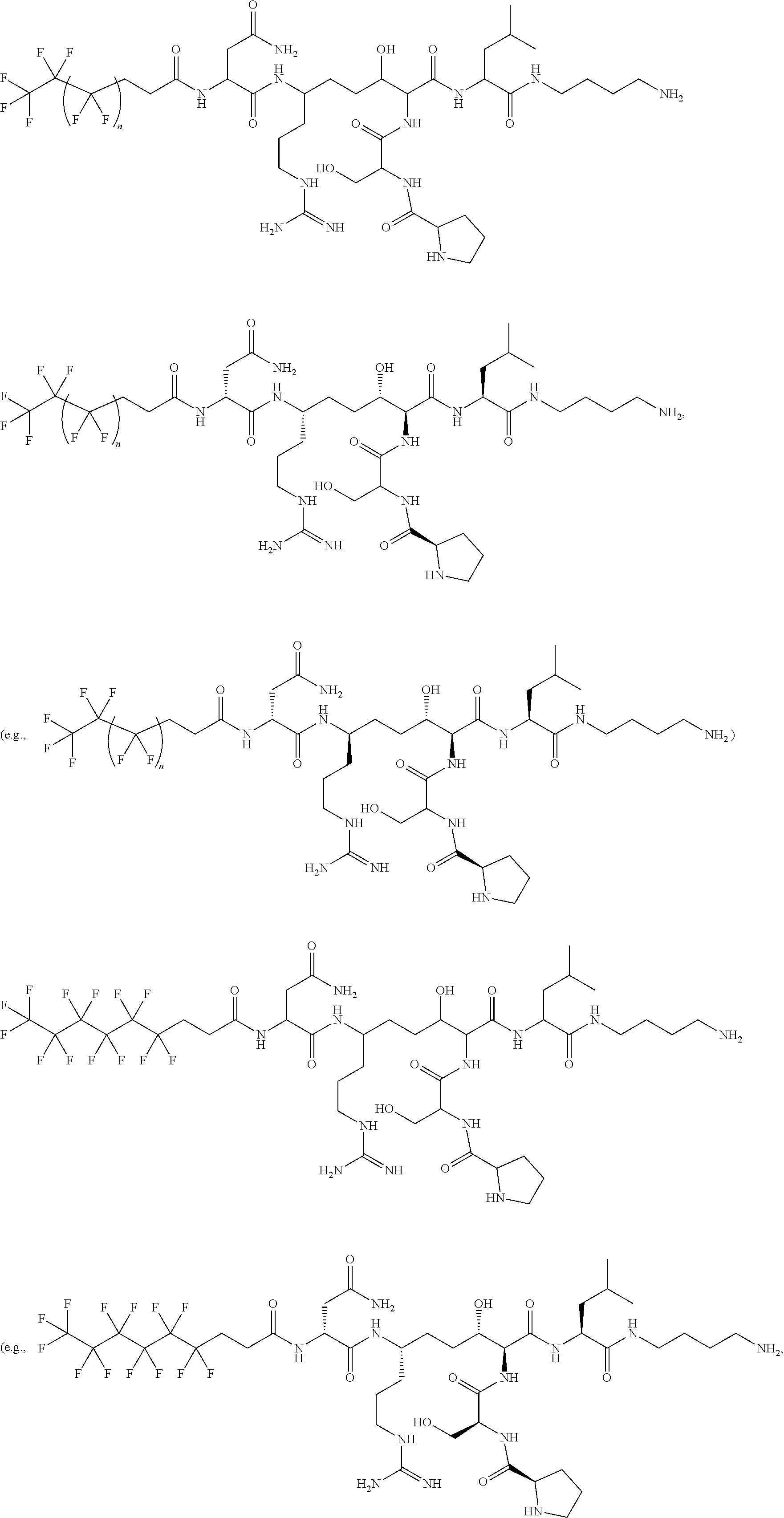

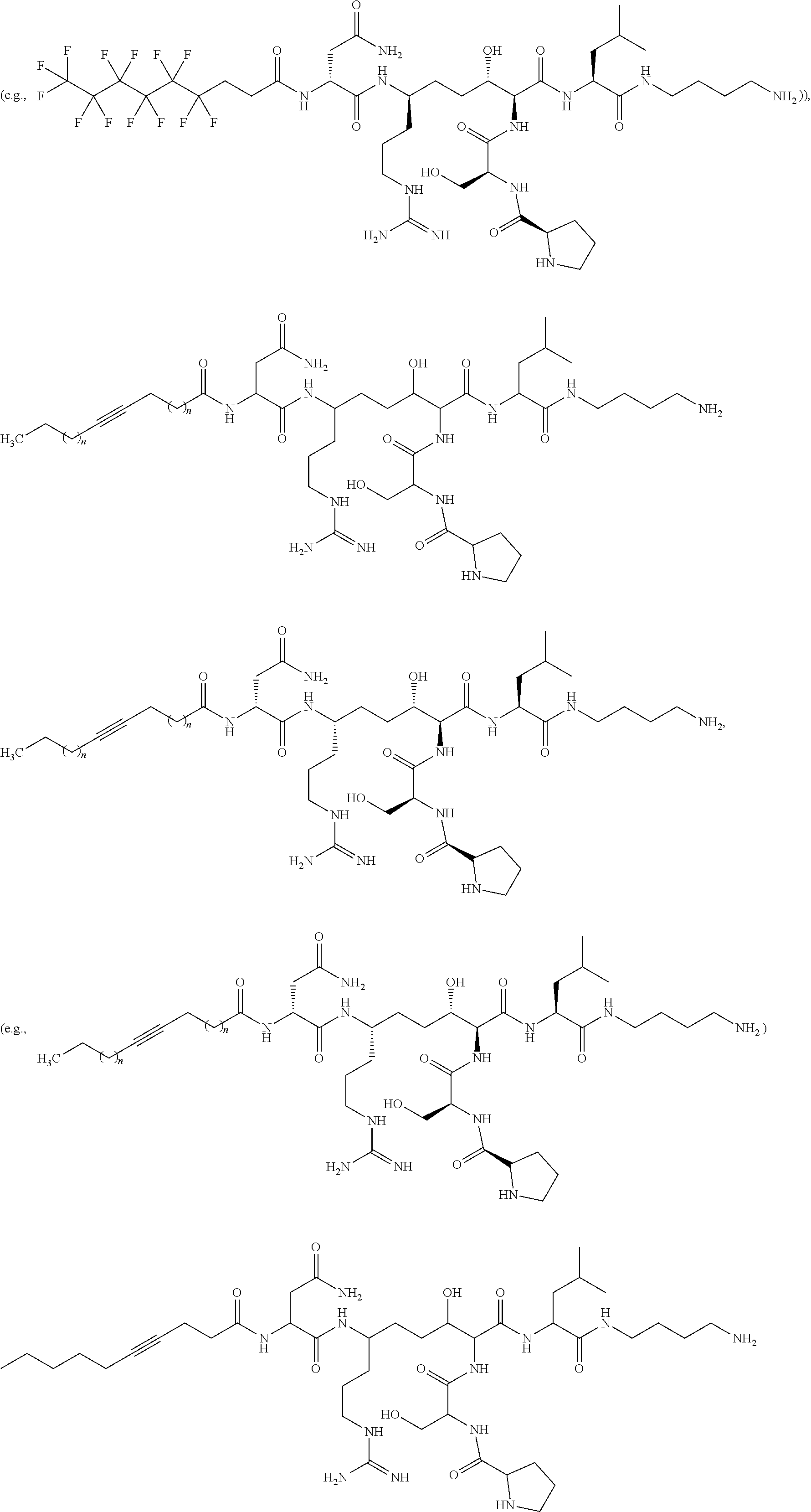

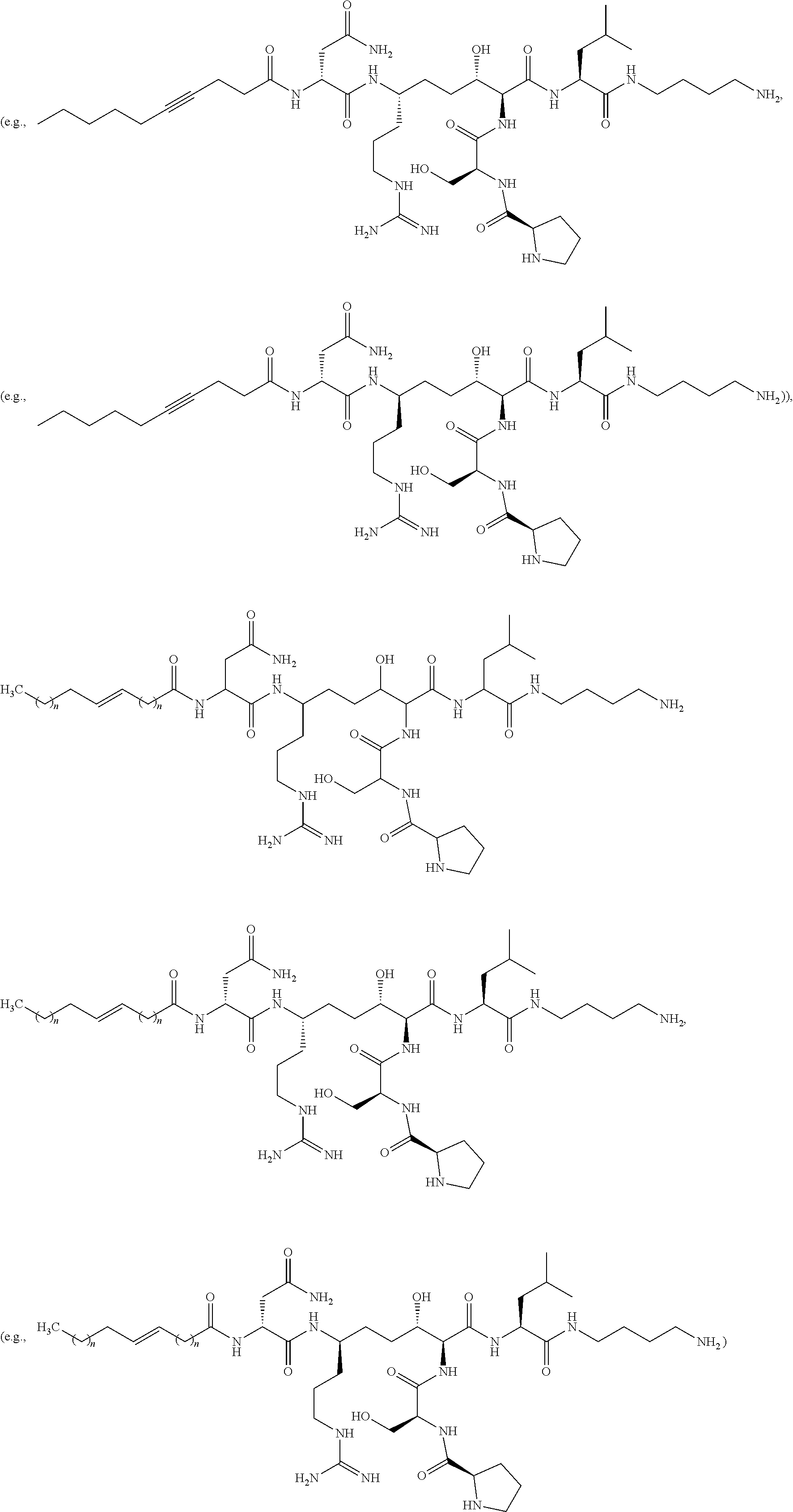

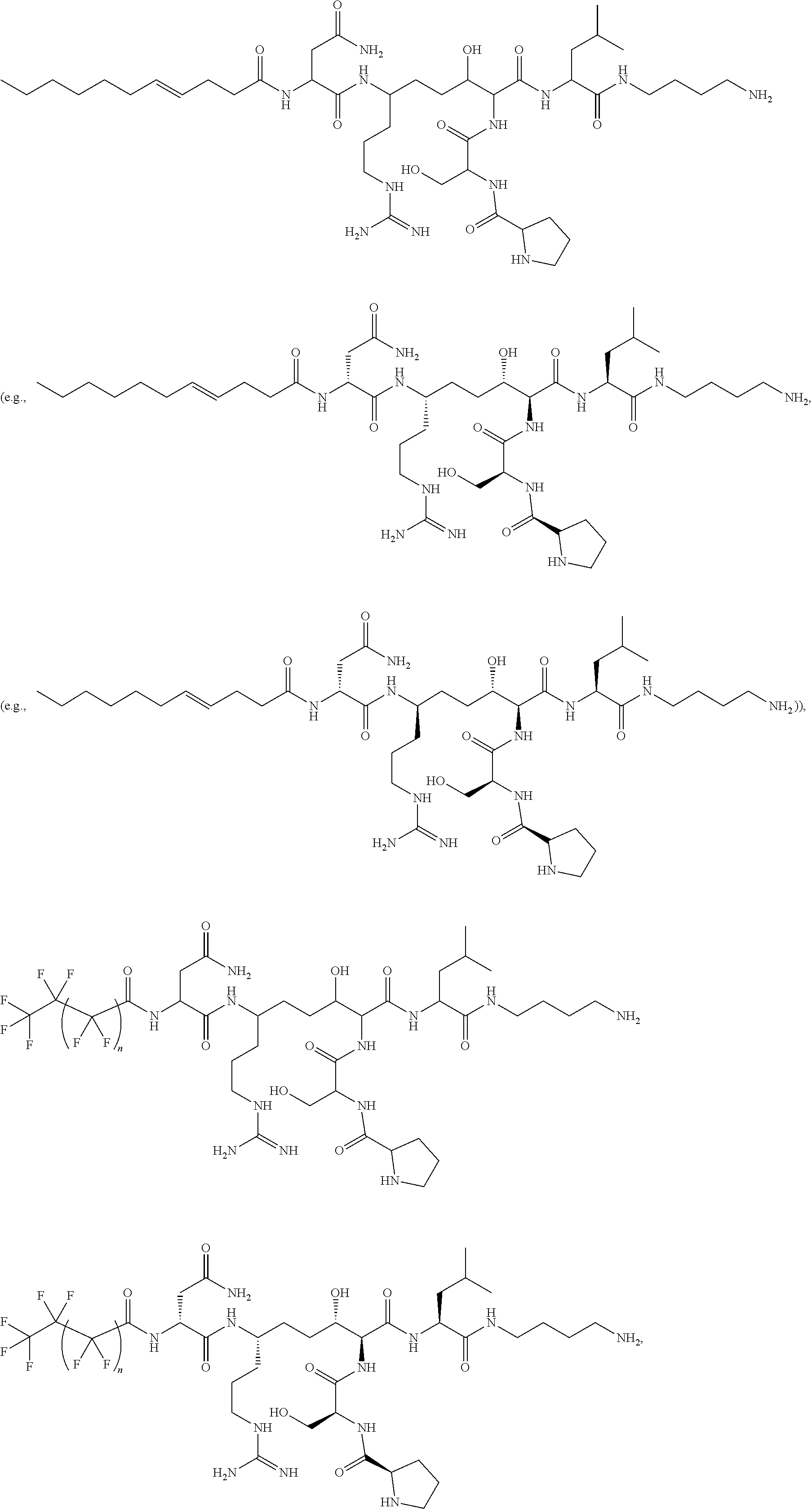

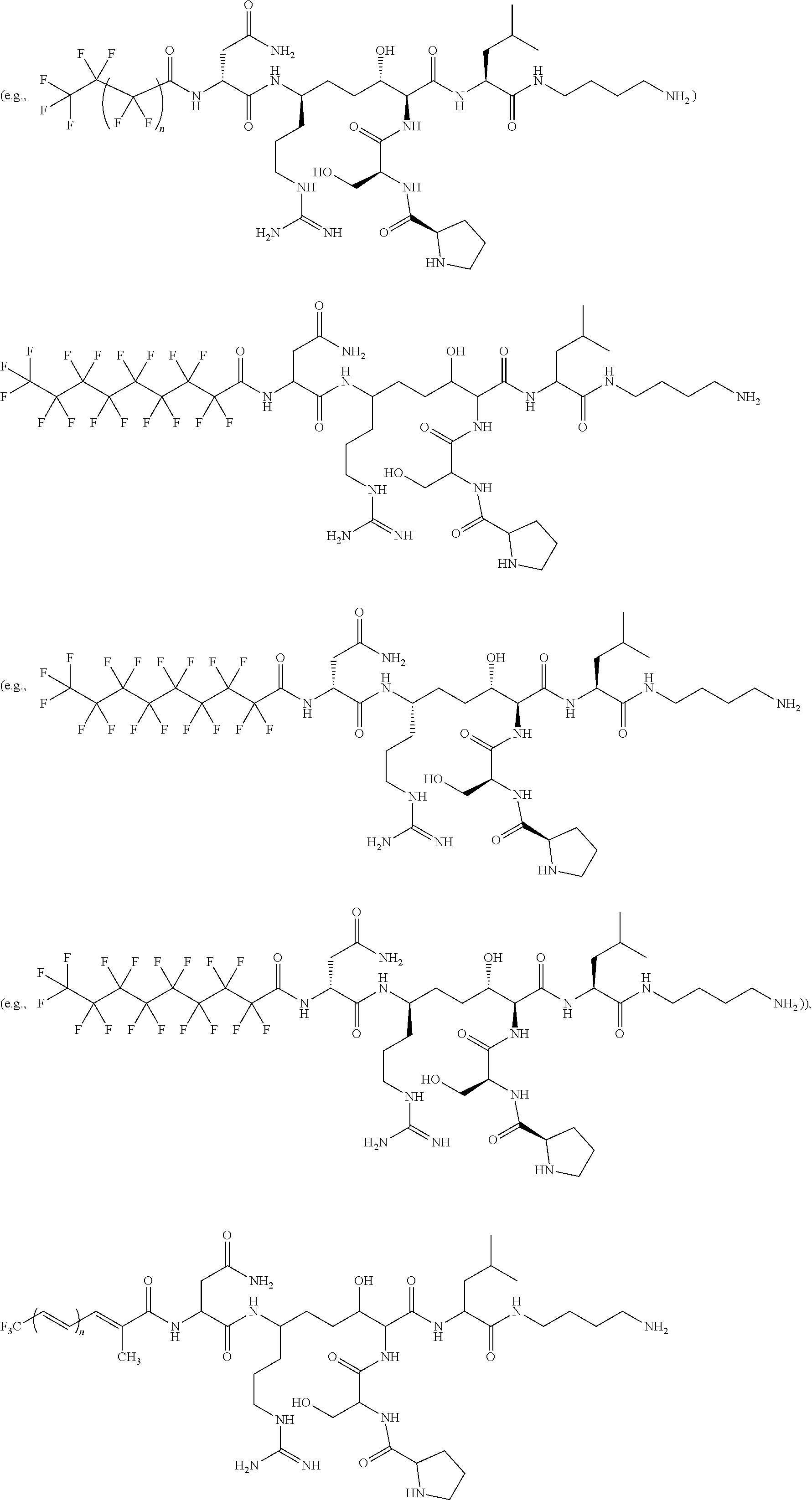

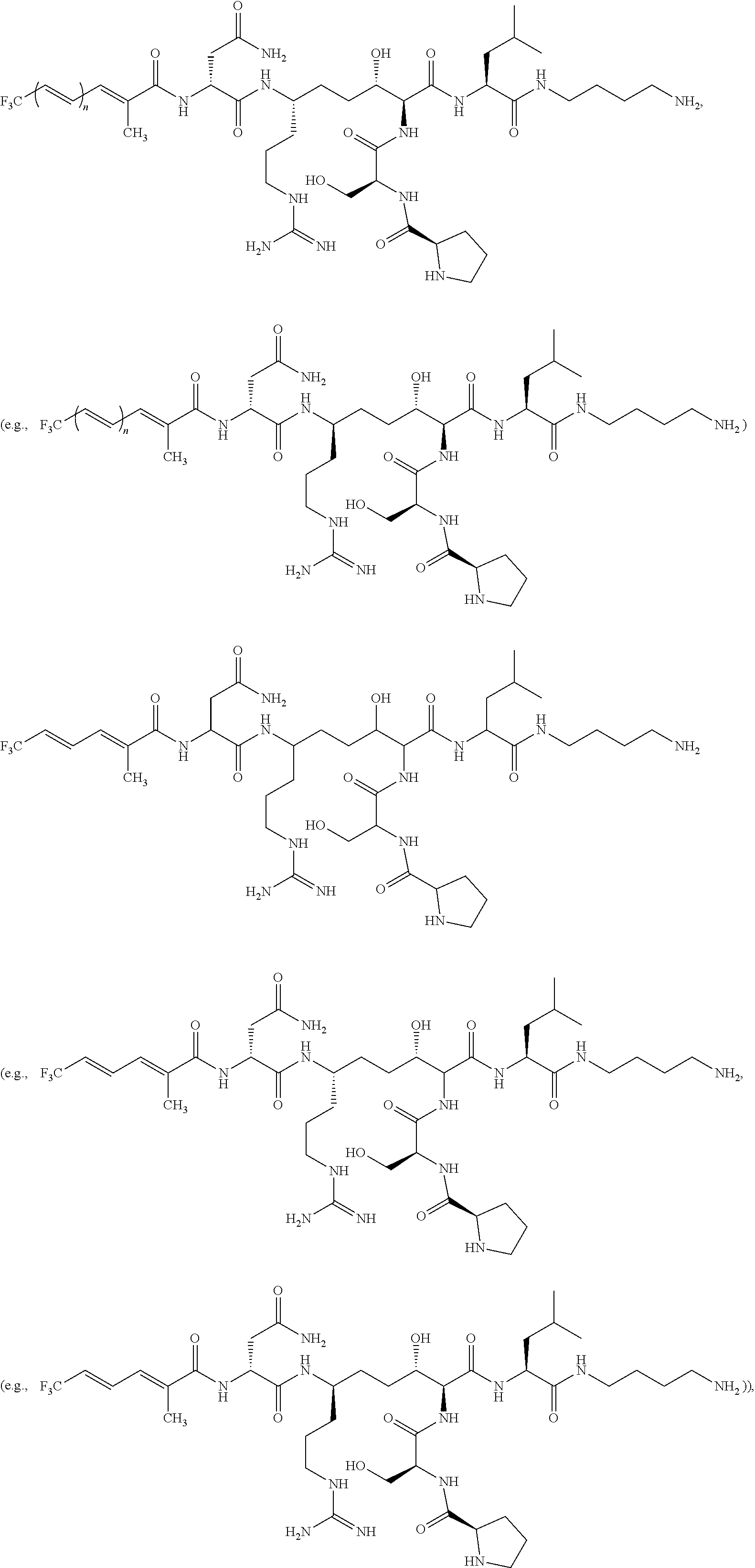

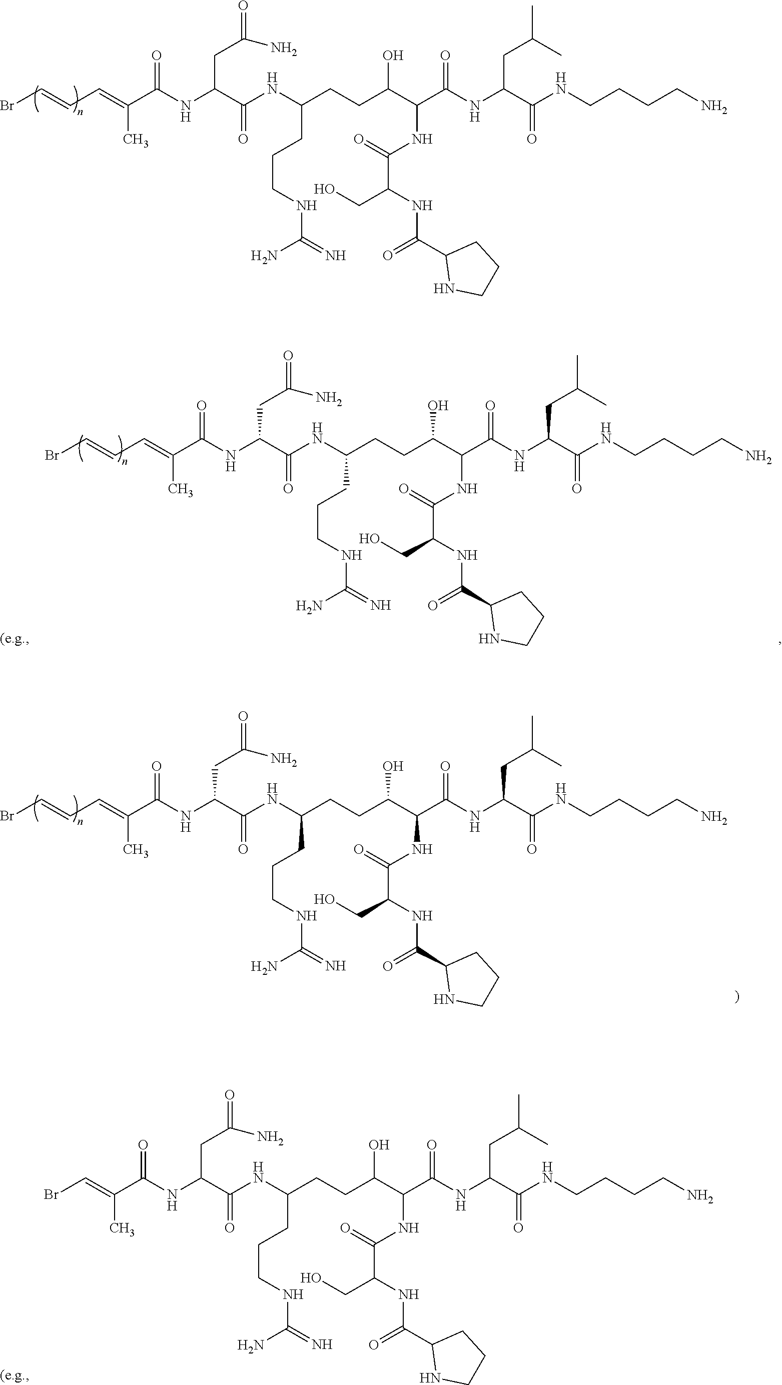

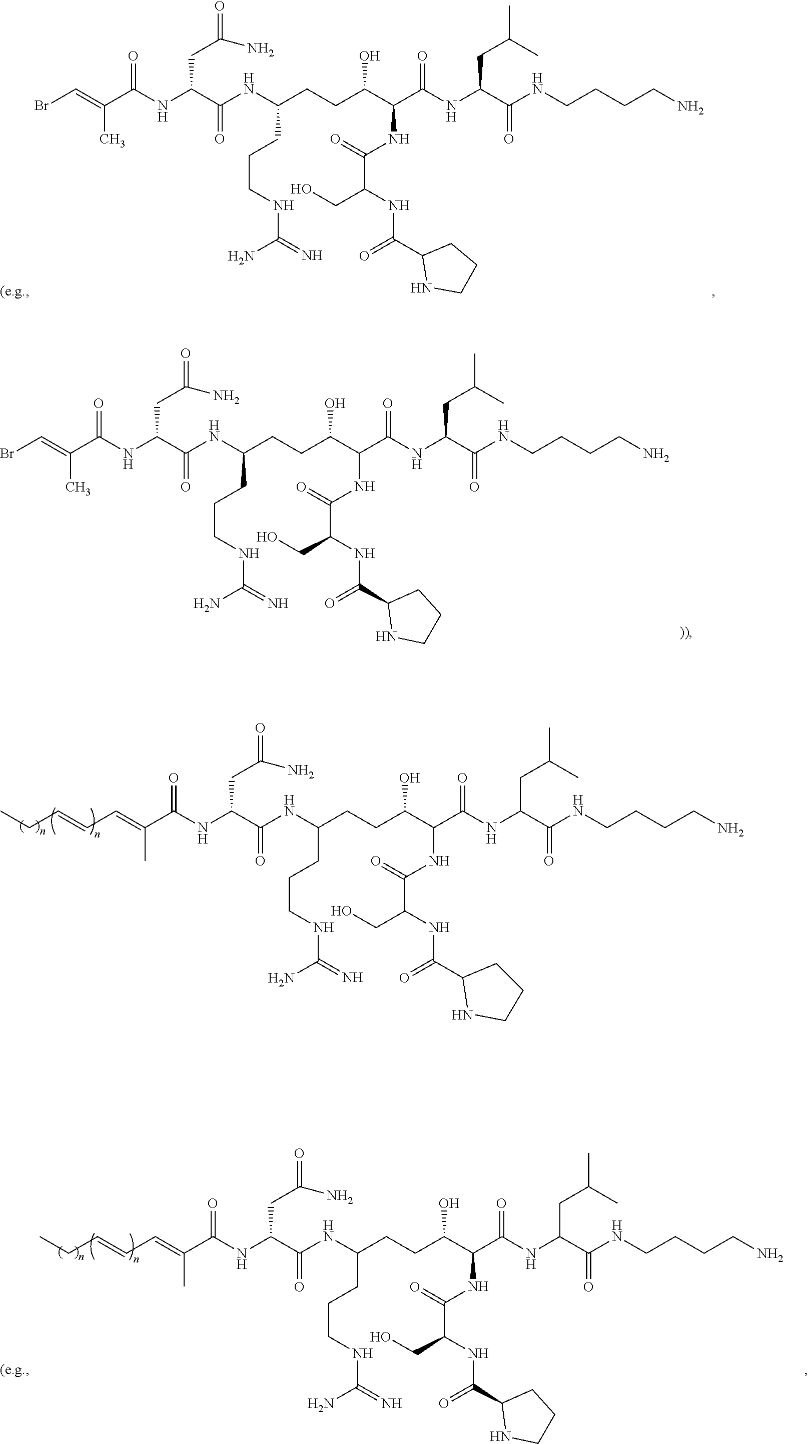

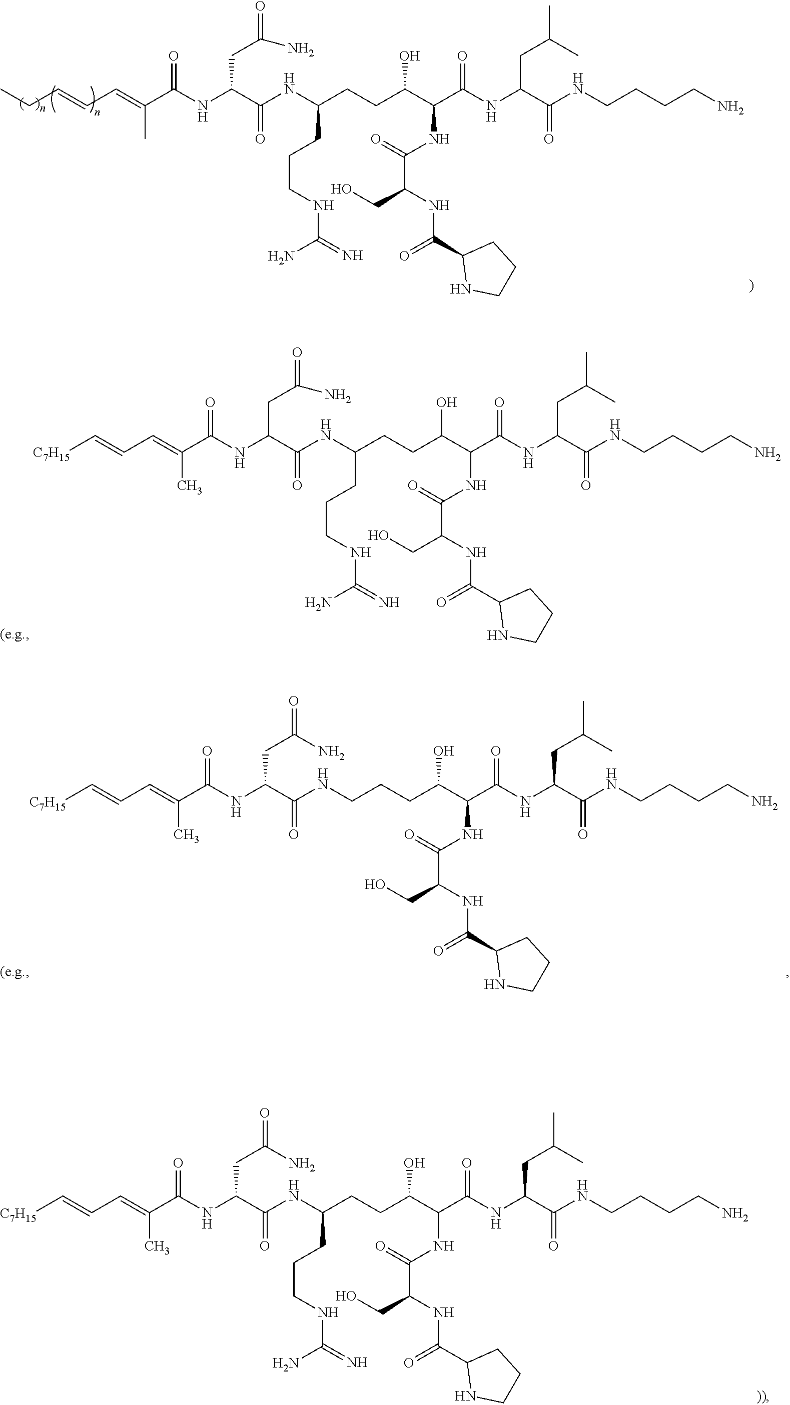

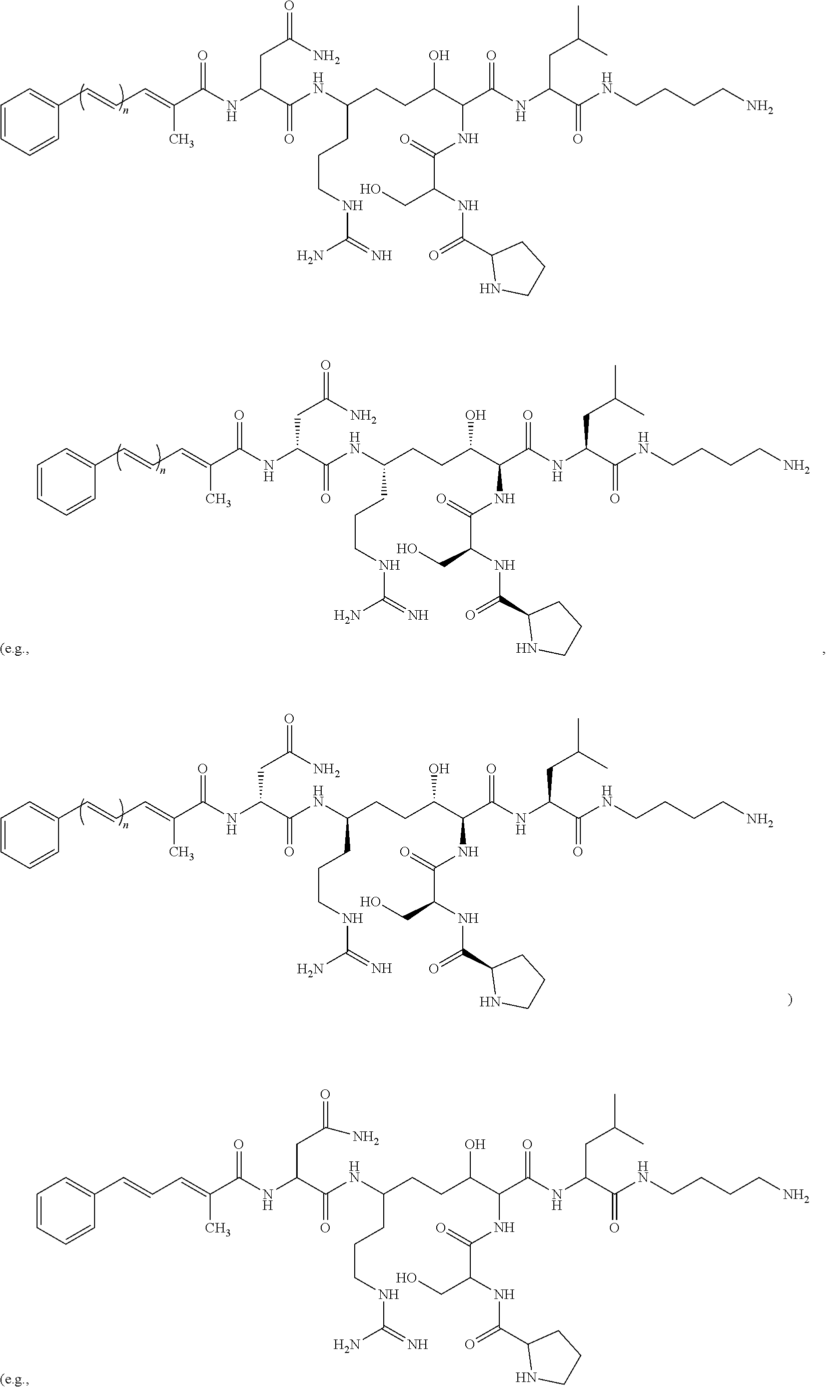

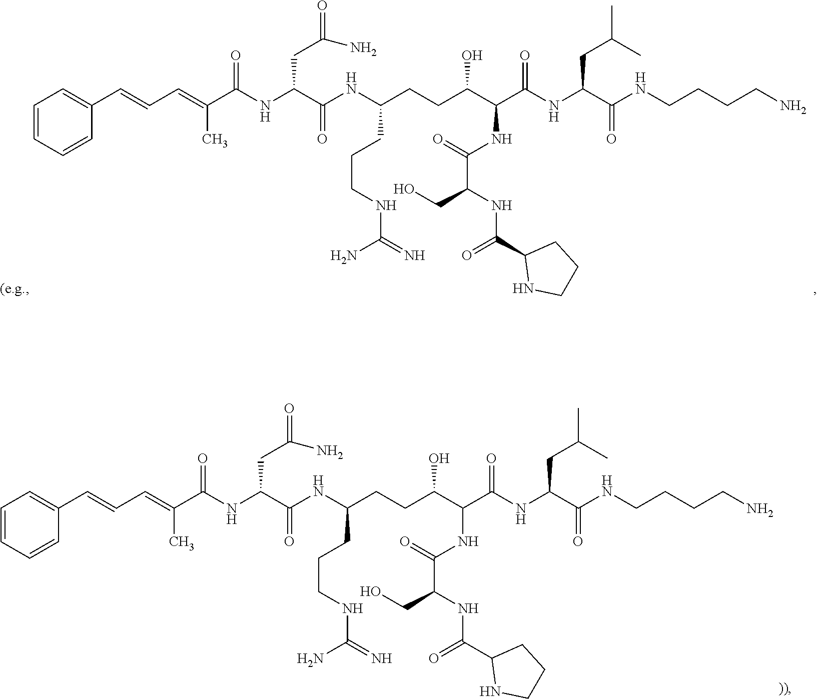 [SMILES: C/C(=C\C=C\c1ccccc1)C(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; C/C(=C\C=C\c1ccccc1)C(=O)N[C@H](CC(N)=O)C(=O)N[C@H](CCCNC(=N)N)CC[C@H](O)C(NC(=O)[C@H](CO)NC(=O)C1CCCN1)C(=O)N[C@@H](CC(C)C)C(=O)NCCCCN]

or any combination thereof, wherein n is 0 to 18.

In various examples, the compound (e.g., the prodrug antibiotic) has the following structure:

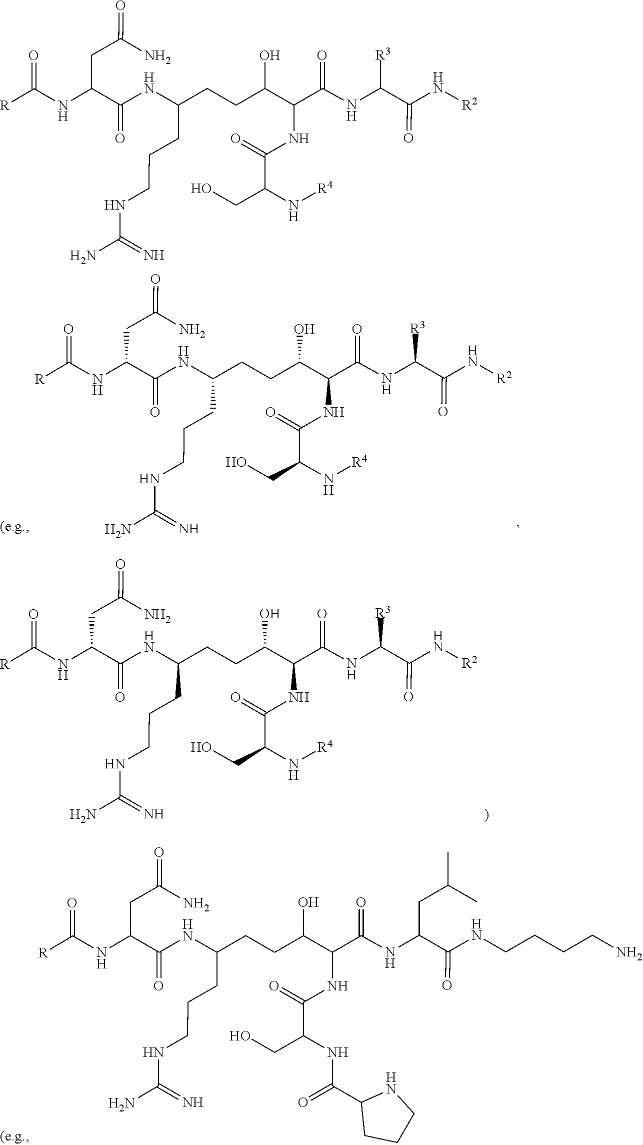

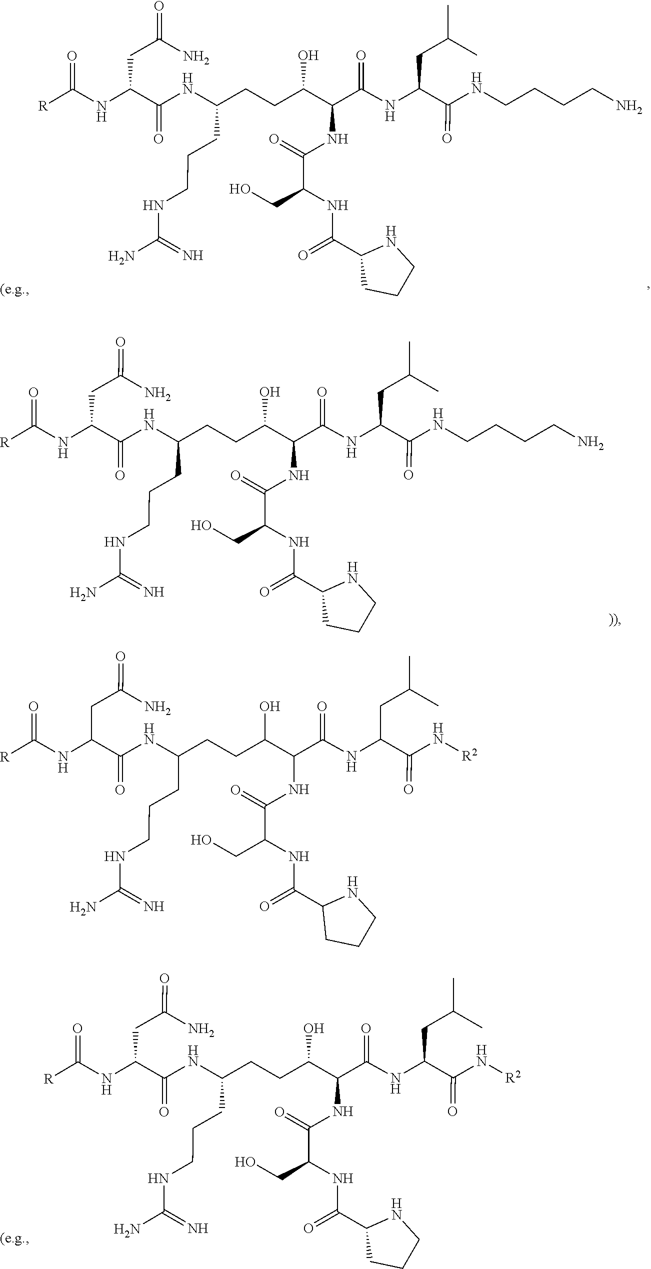

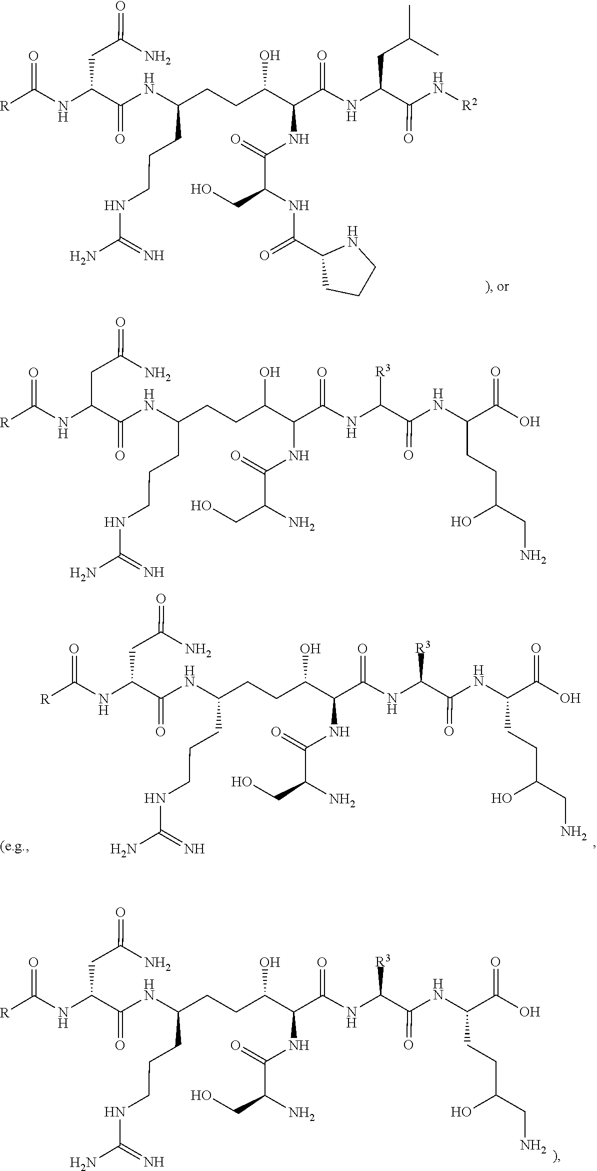

where is R may be an aliphatic group 1 to 25 carbons atoms in length (e.g., I to 22 carbon atoms in length, 1 to 18 carbon atoms in length, or 7 to 25 carbon atoms in length) (e.g., 1, 2, 3, 4, 5, 6, 7, 8, 9, 10, 11, 12, 13, 14, 15, 16, 17, 18, 19, 20, 21, 22, 23, 24, or 25 carbon atoms in length). The aliphatic group may be substituted or unsubstituted. The aliphatic group may have one or more aromatic rings (e.g., aryl or heteroaryl groups), one or more pi bonds (e.g., one or more double bonds and/or one or more triple bonds), one or more hydroxy groups, and/or one or more halogens (e.g., fluoro groups). The one or more pi bonds may be conjugated. For example, aromatic rings may be pendent substituents, terminal substituents, or the aromatic rings may interrupt a linear aliphatic chain such that a portion of the aliphatic group is one substituent on the aromatic ring and the remaining portion of the aliphatic chain is a different substituent on the aromatic ring

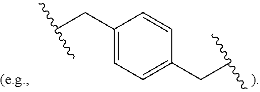 [SMILES: *Cc1ccc(C*)cc1]

The aromatic rings may have one or more additional substituents. For example, R may have the following structure:

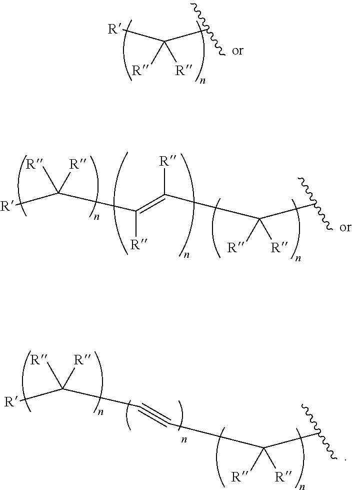

R′ is hydrogen, a halogen (e.g., bromo, fluoro, or the like), an aryl group (e.g., phenyl, napthyl, or the like), a methyl group, halogenated methyl group (e.g., —CF₃), an amino group, or a hydroxy, or the like. An R′ group may be unsubstituted or further substituted. R″ is hydrogen, a halogen (e.g., bromo, fluoro, or the like), an aryl group (e.g., phenyl, napthyl, or the like), a methyl group, halogenated methyl group (e.g., —CF₃), a hydroxy, or the like. Each R″ may be the same or different. As an illustrative example, on a carbon atom substituted with two R″ groups, one R″ is hydrogen, and one R″ is methyl. In another illustrative example, if R is

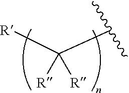

and n is 2, each of the four resulting R″ groups may be the same or different. For example, one R″ is a methyl, one R″ is hydrogen, one R″ is fluoro, and the final R″ is bromo. Other combinations of substituents on these genuses are contemplated by this disclosure. Each n is independently 0 to 18 (e.g., 0, 1, 2, 3, 4, 5, 6, 7, 8, 9, 10, 11, 12, 13, 14, 15, 16, 17, 18). In various examples, R may have the following structure:

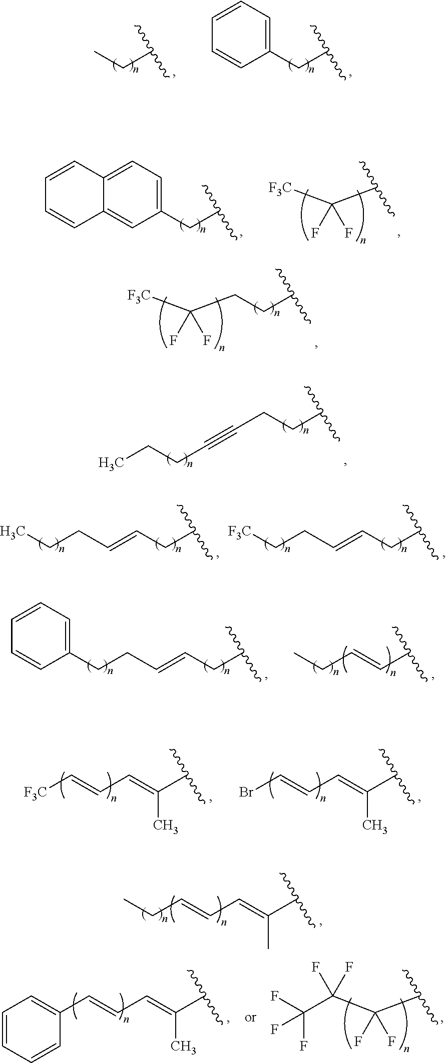

wherein each n is independently 0 to 18 (e.g., 0, 1, 2, 3, 4, 5, 6, 7, 8, 9, 10, 11, 12, 13, 14, 15, 16, 17, 18). Additional examples of R groups include, but are not limited to:

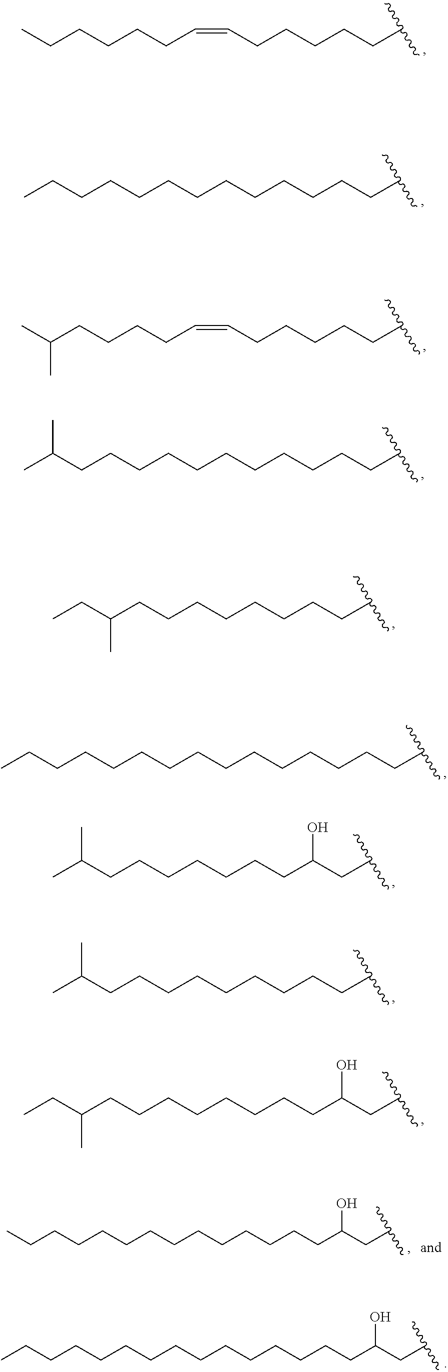

The R² group may be an amino acid residue (e.g., a canonical or noncanonical amino acid residue), a substituted or unsubstituted aliphatic group, or H. Examples of R² groups include, but are not limited to,

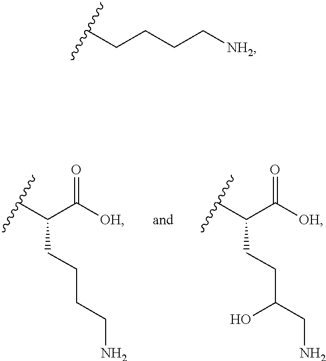 [SMILES: *CCCCN ; *[C@@H](CCCCN)C(=O)O ; *[C@@H](CCC(O)CN)C(=O)O]

The R³ group may be a substituted or unsubstituted aliphatic group. In various other examples, the R³ is the side chain of an amino acid (e.g., a canonical or noncanonical amino acid). For example, R³ may be the side chain of a hydrophobic amino acid. Examples of R³ groups include, but are not limited to,

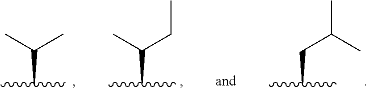

The R⁴ group may be a substituted or unsubstituted acyl group or H. In various examples, the R⁴ group is an amino acid residue (e.g., a canonical or noncanonical amino acid residue). For example, R⁴ may be proline.

In various examples, the compounds (e.g., the prodrug antibiotic) of the preceding paragraph are converted in vivo to the following compounds, respectively:

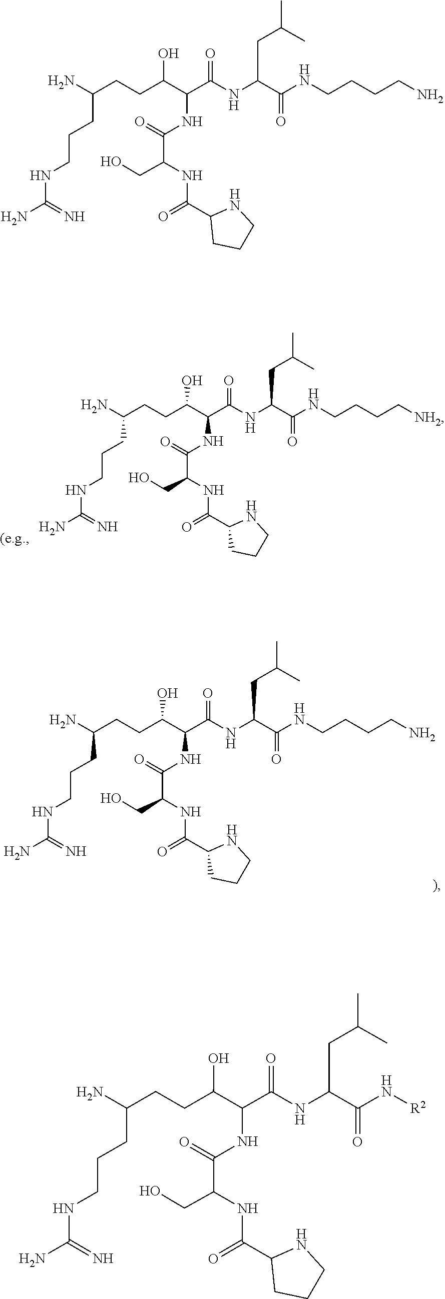

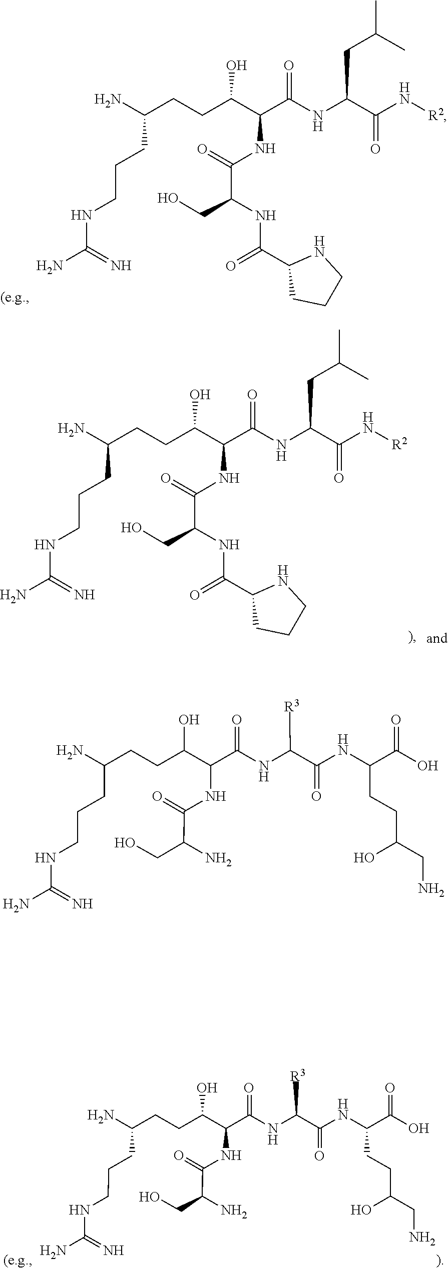

Examples of compounds (e.g., the prodrug antibiotic) include, but are not limited to,

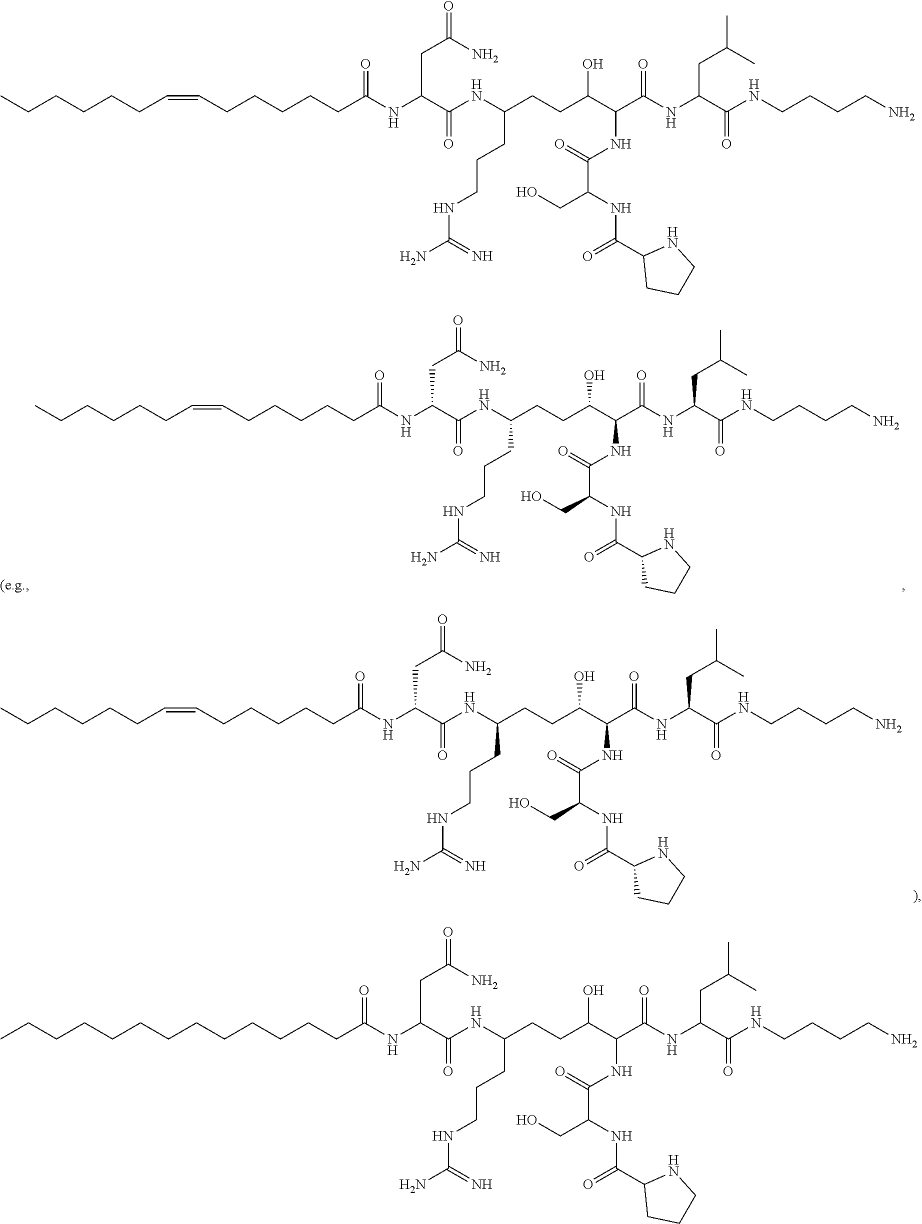

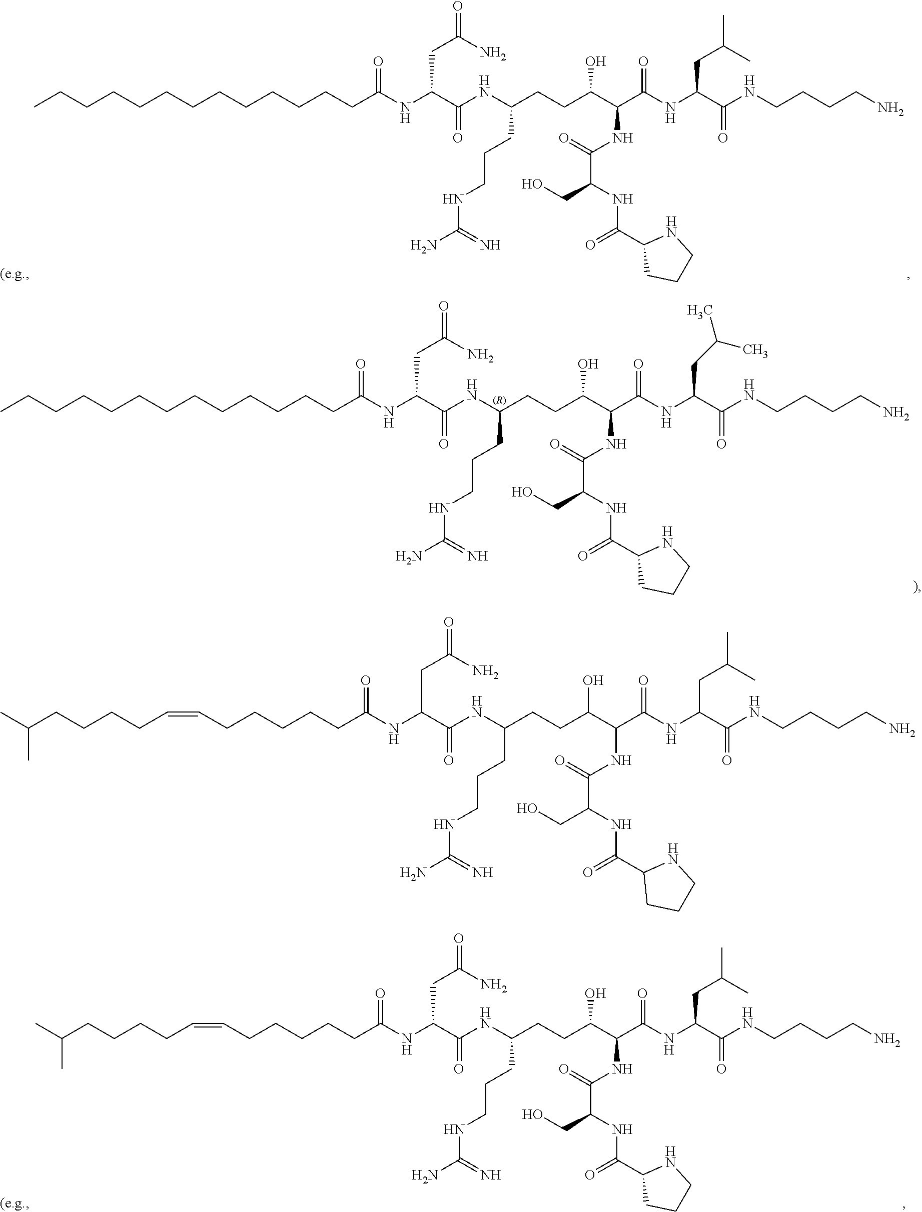

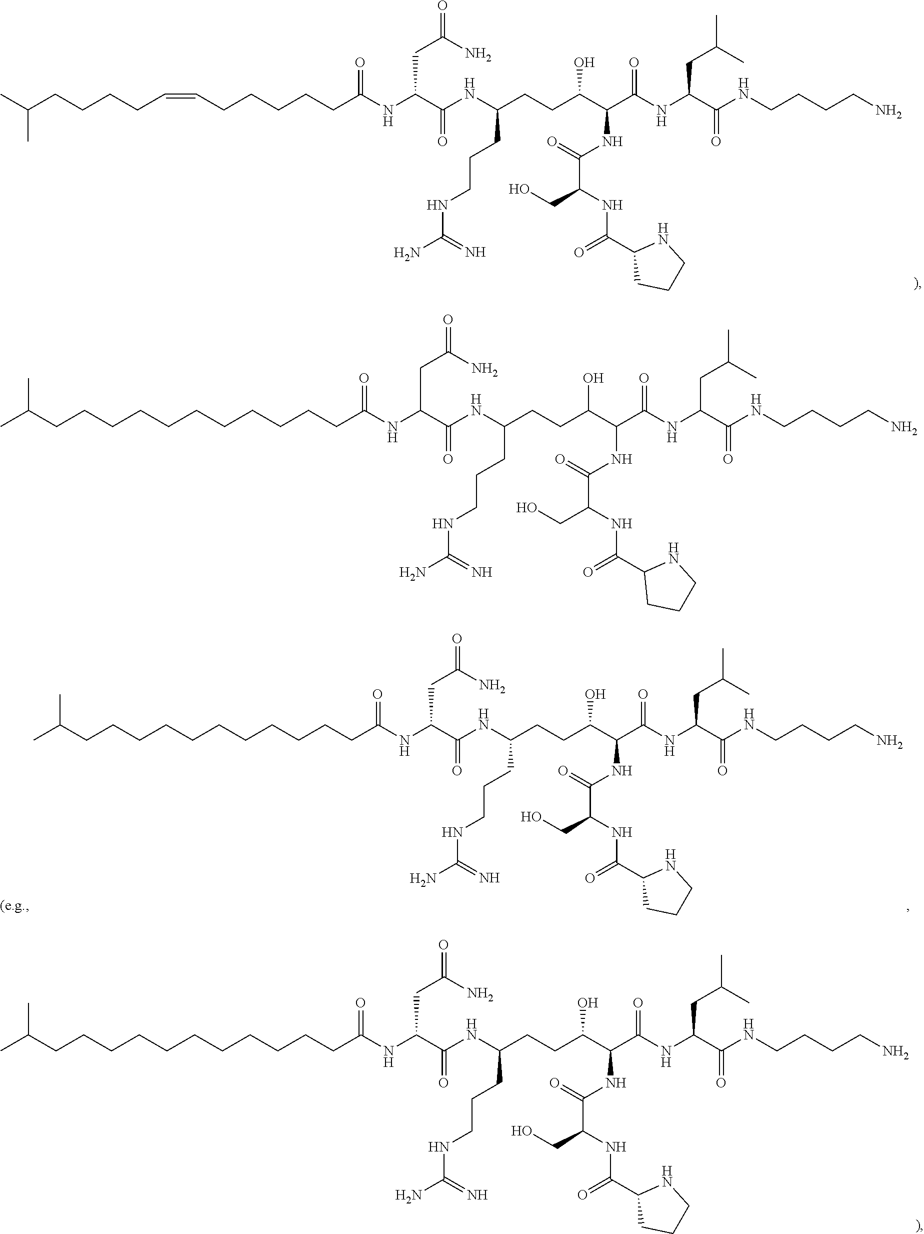

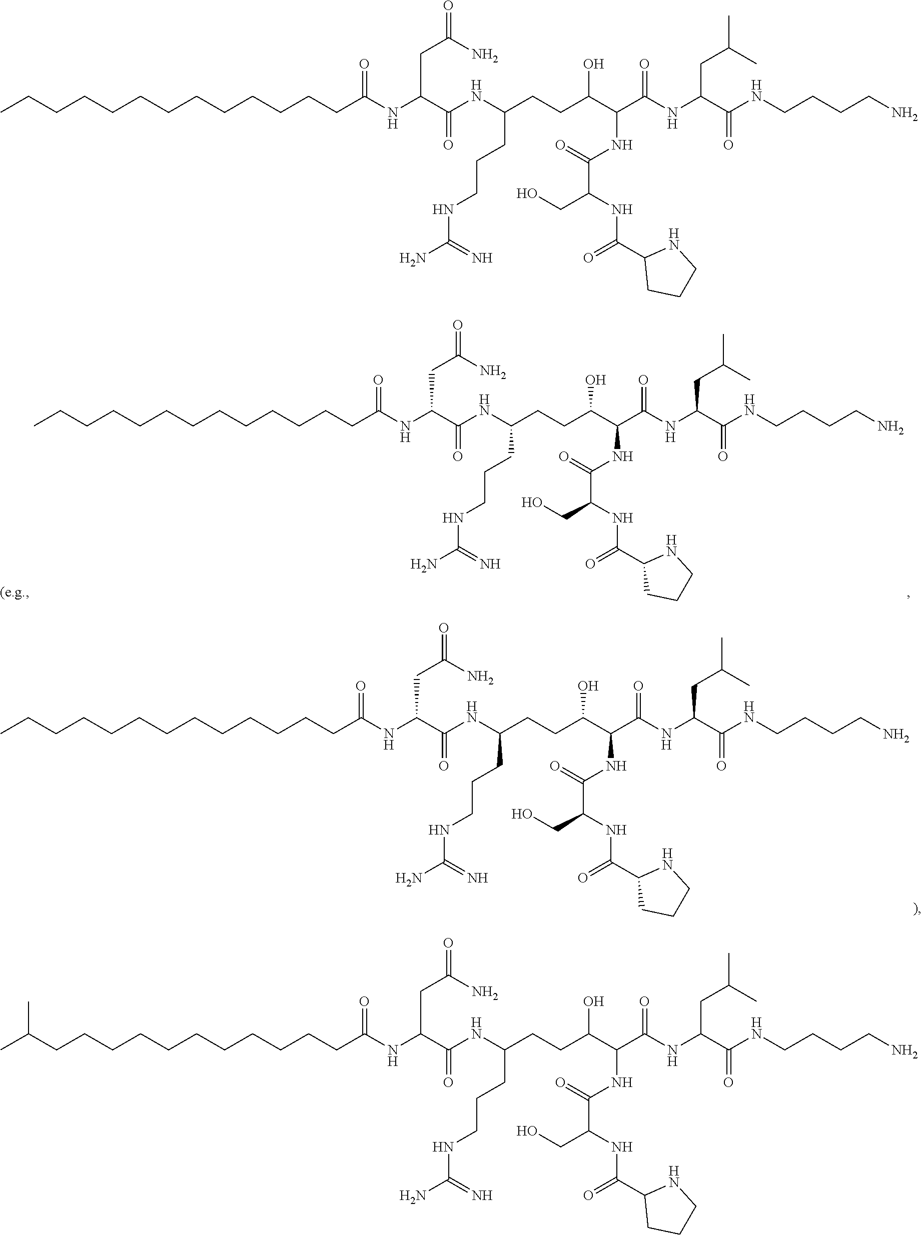 [SMILES: CCCCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CCCCCCCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CCCCCCCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CC(C)CCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN]

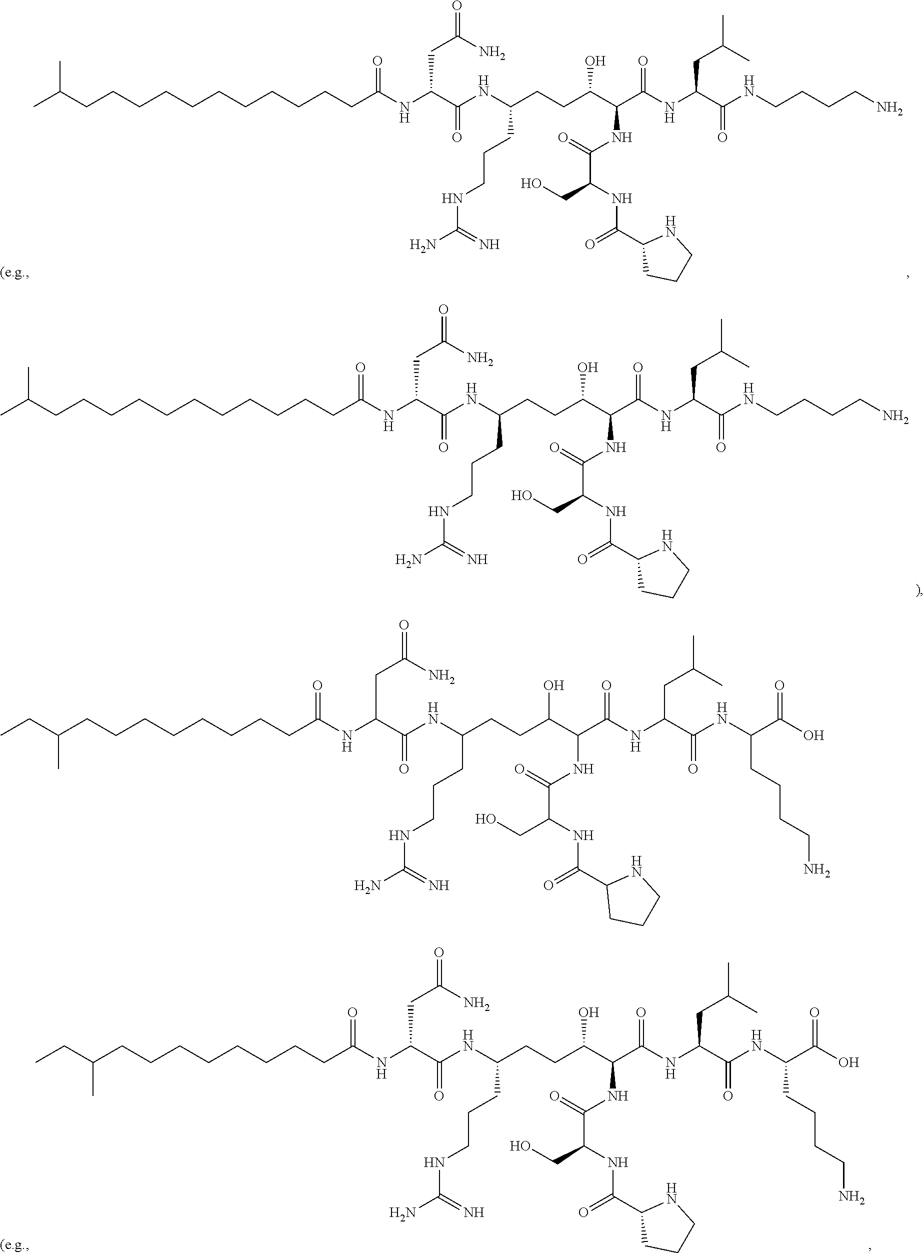

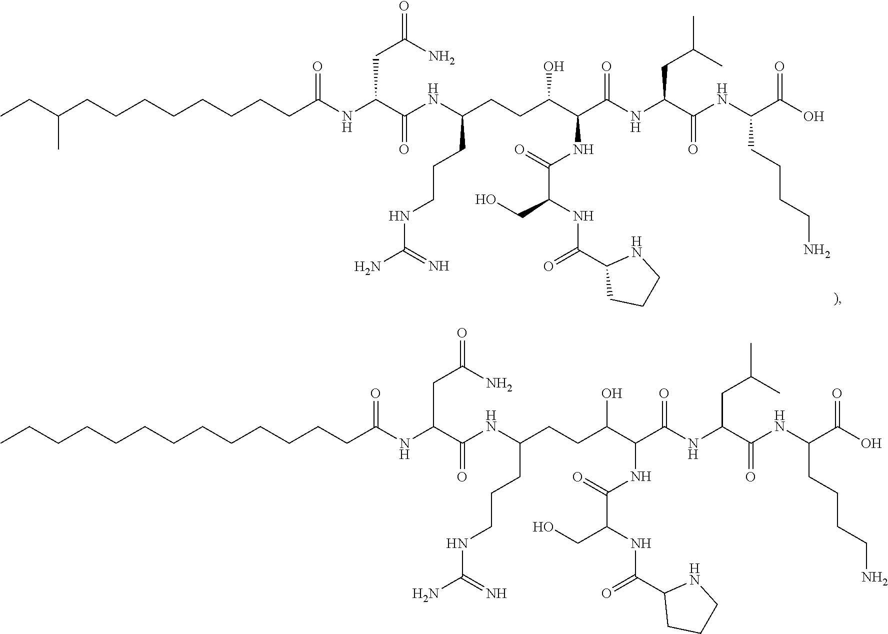 [SMILES: CCC(C)CCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)N[C@@H](CCCCN)C(=O)O ; CCCCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NC(CCCCN)C(=O)O]

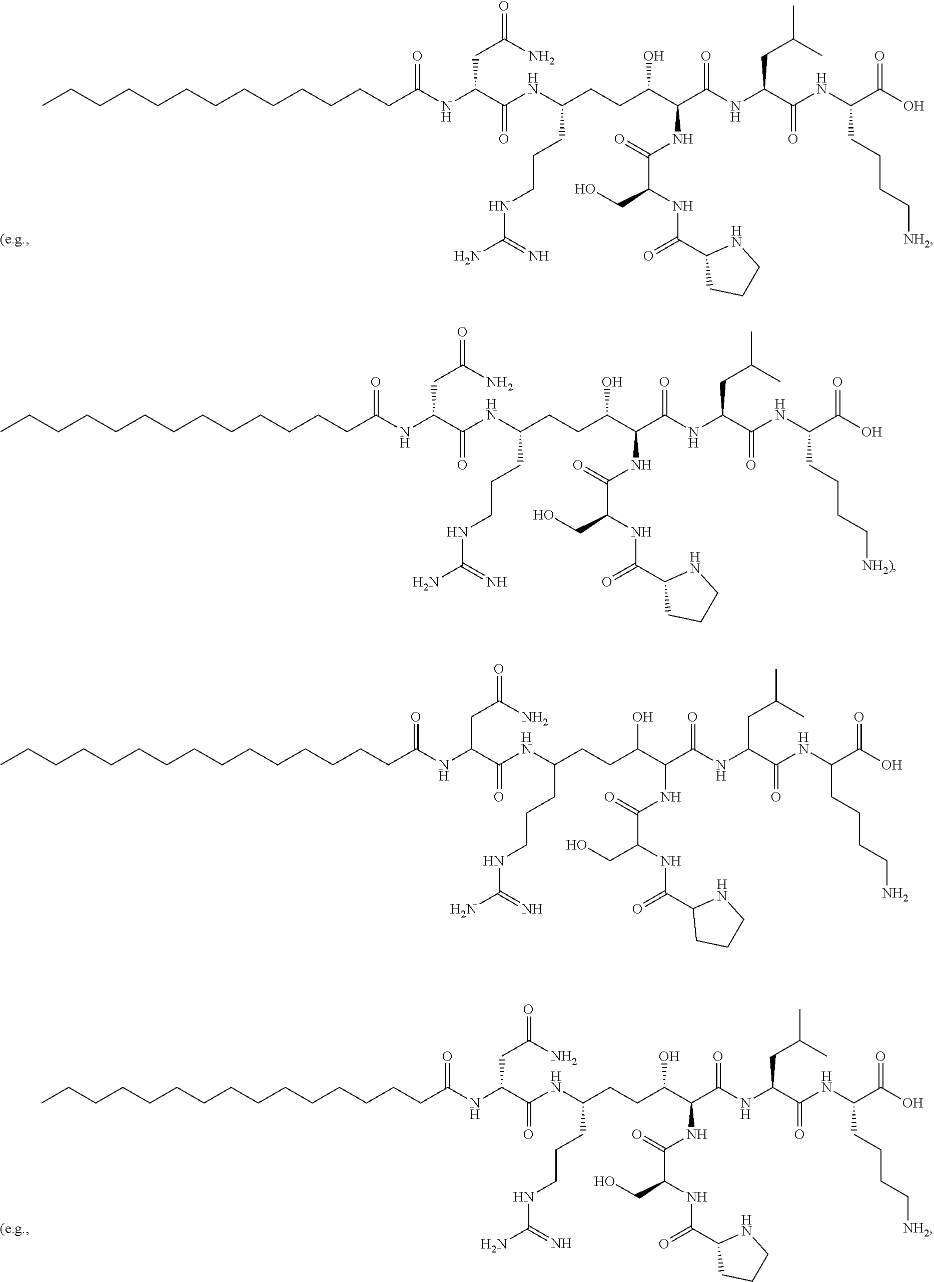

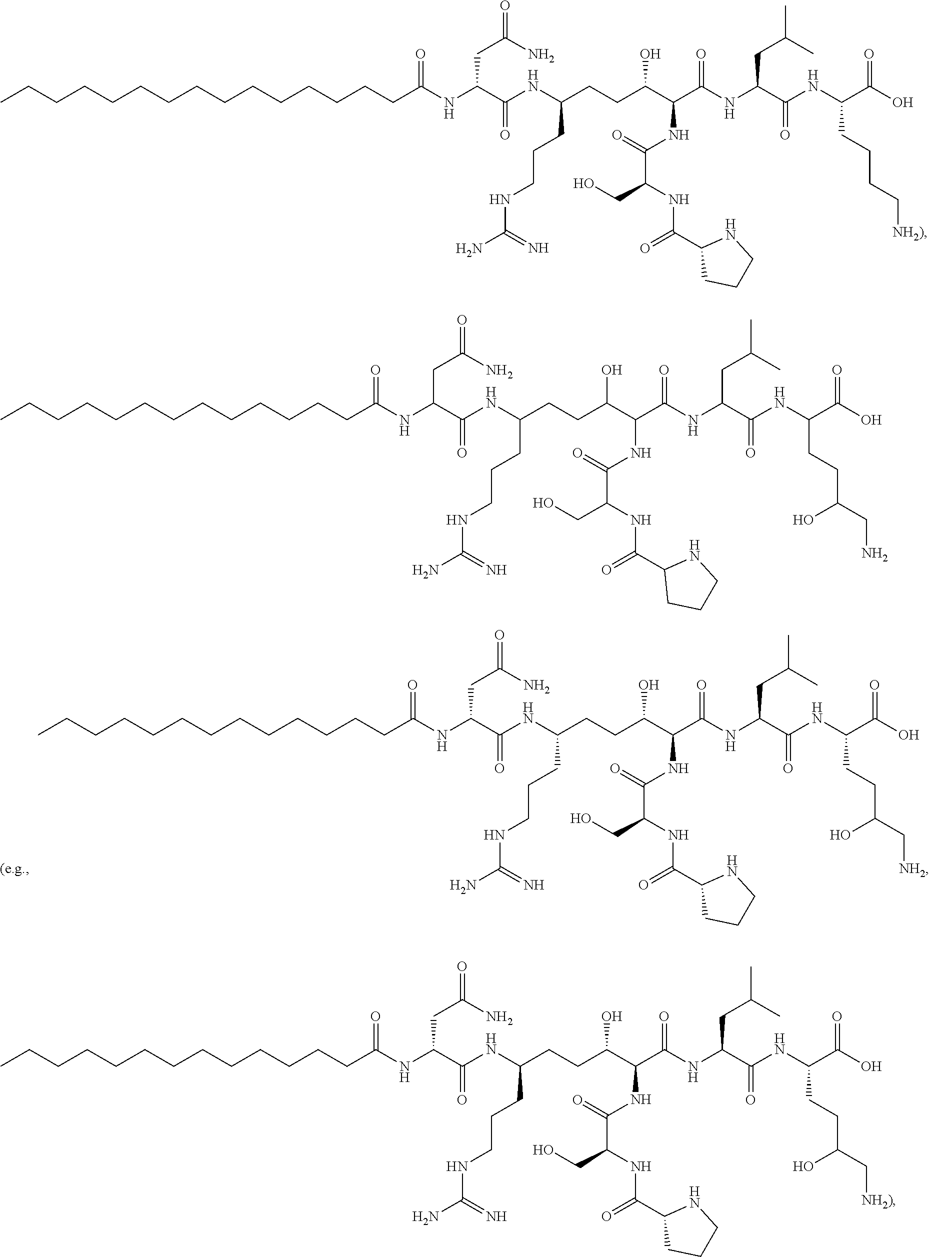

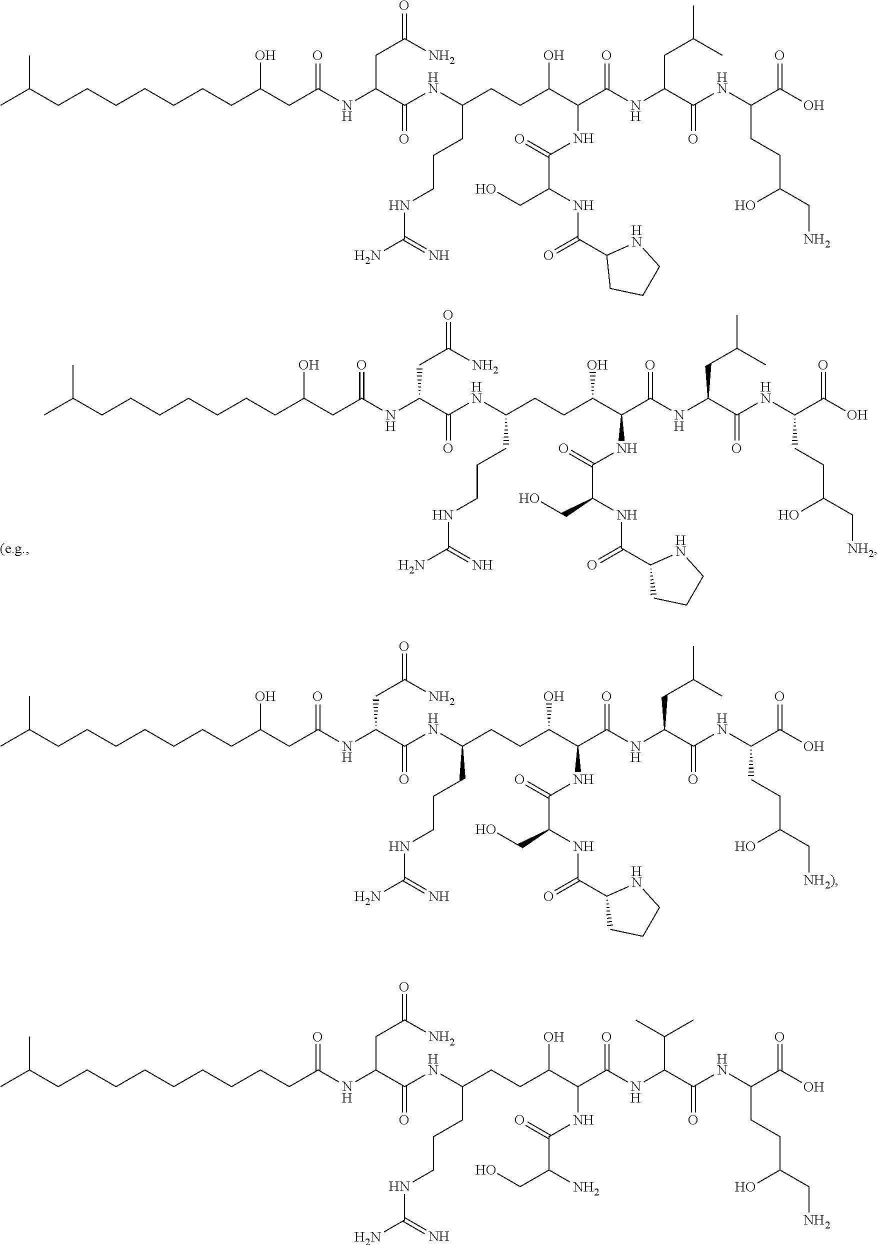

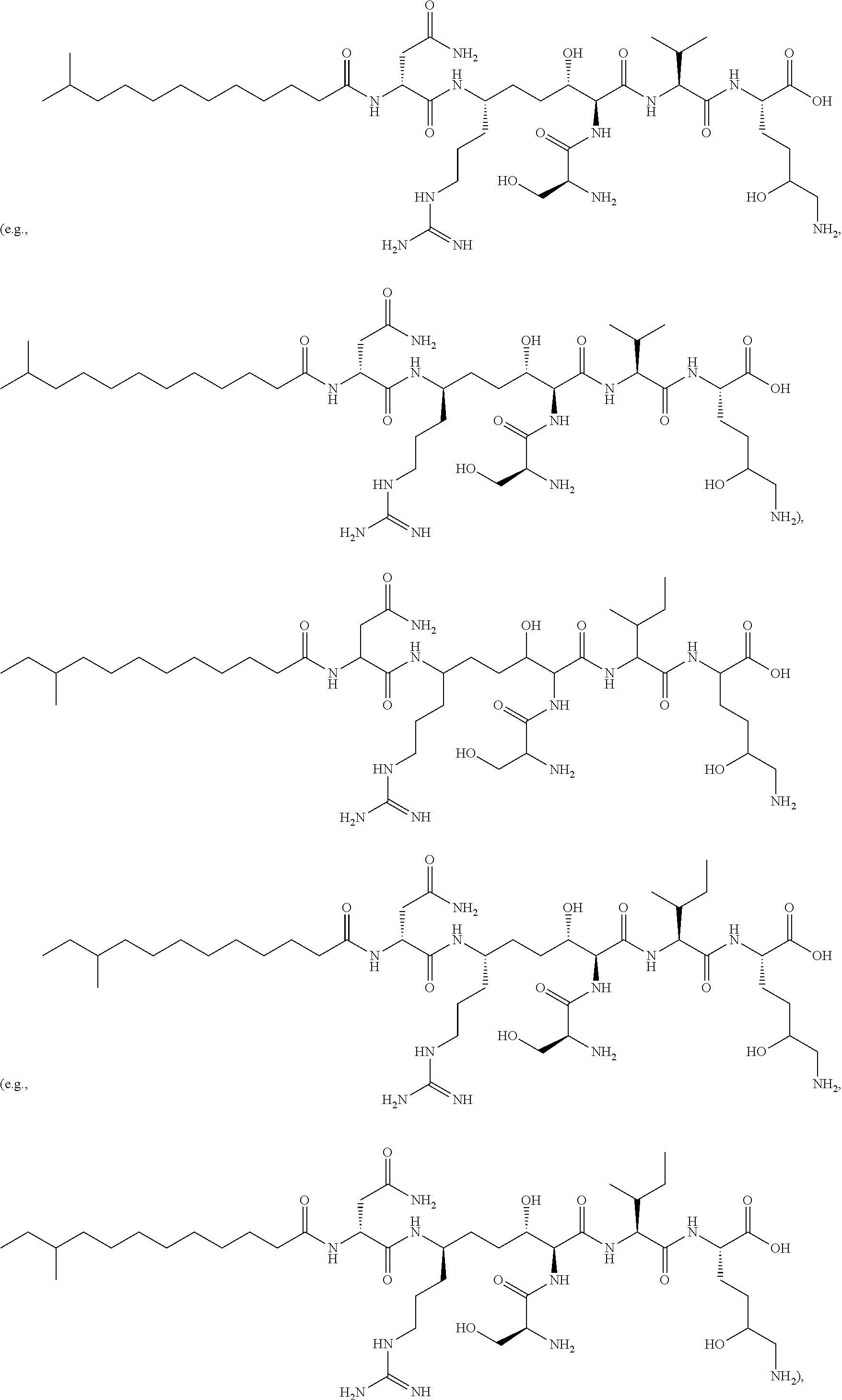

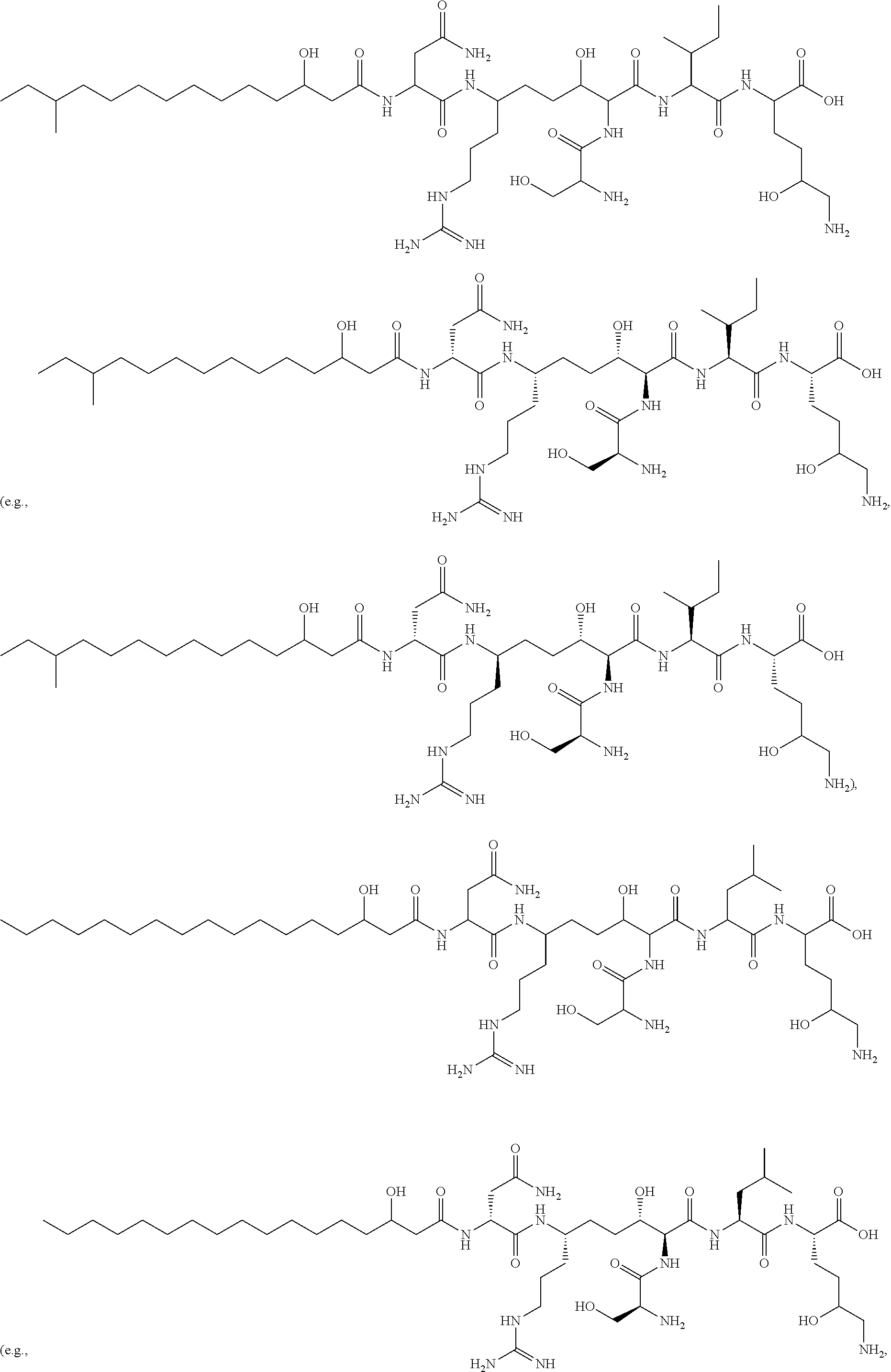

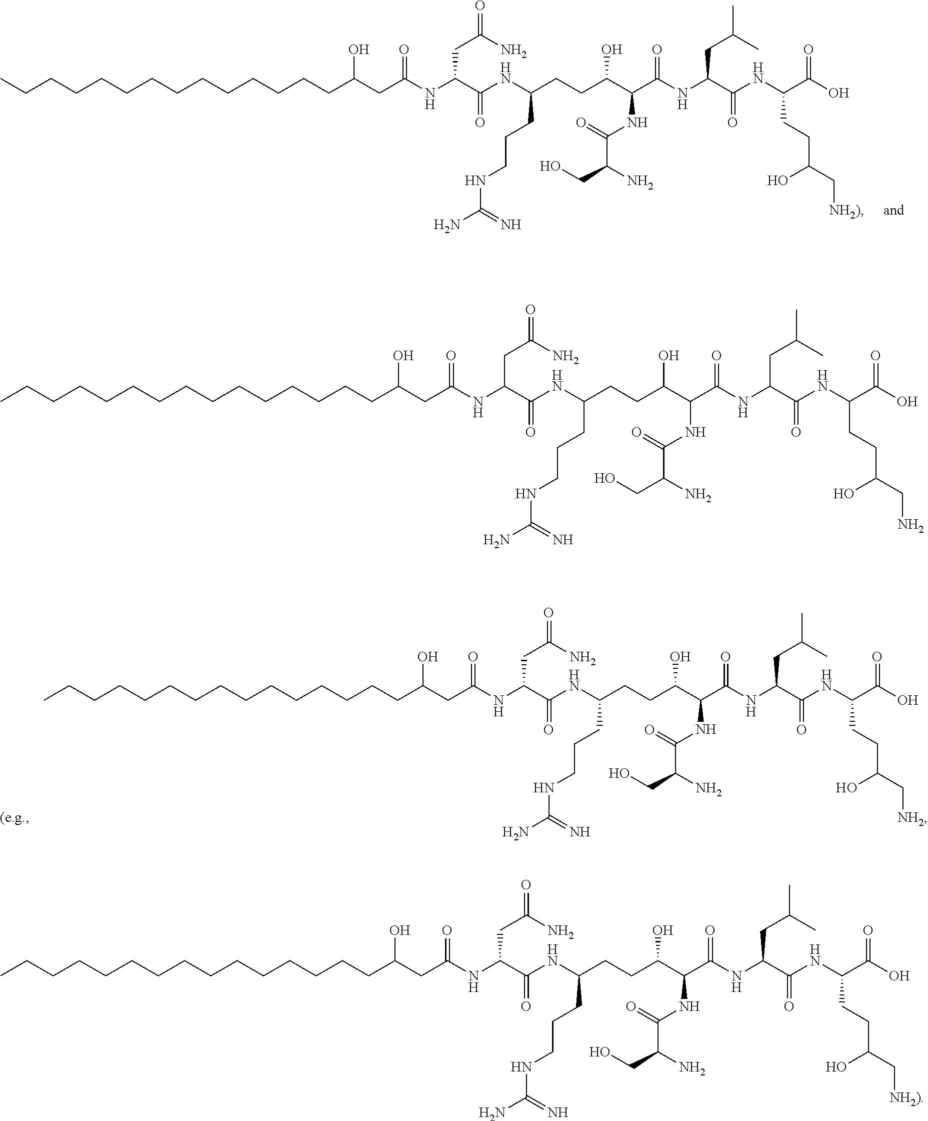

In various examples, the following compounds (e.g., prodrug antibiotics):

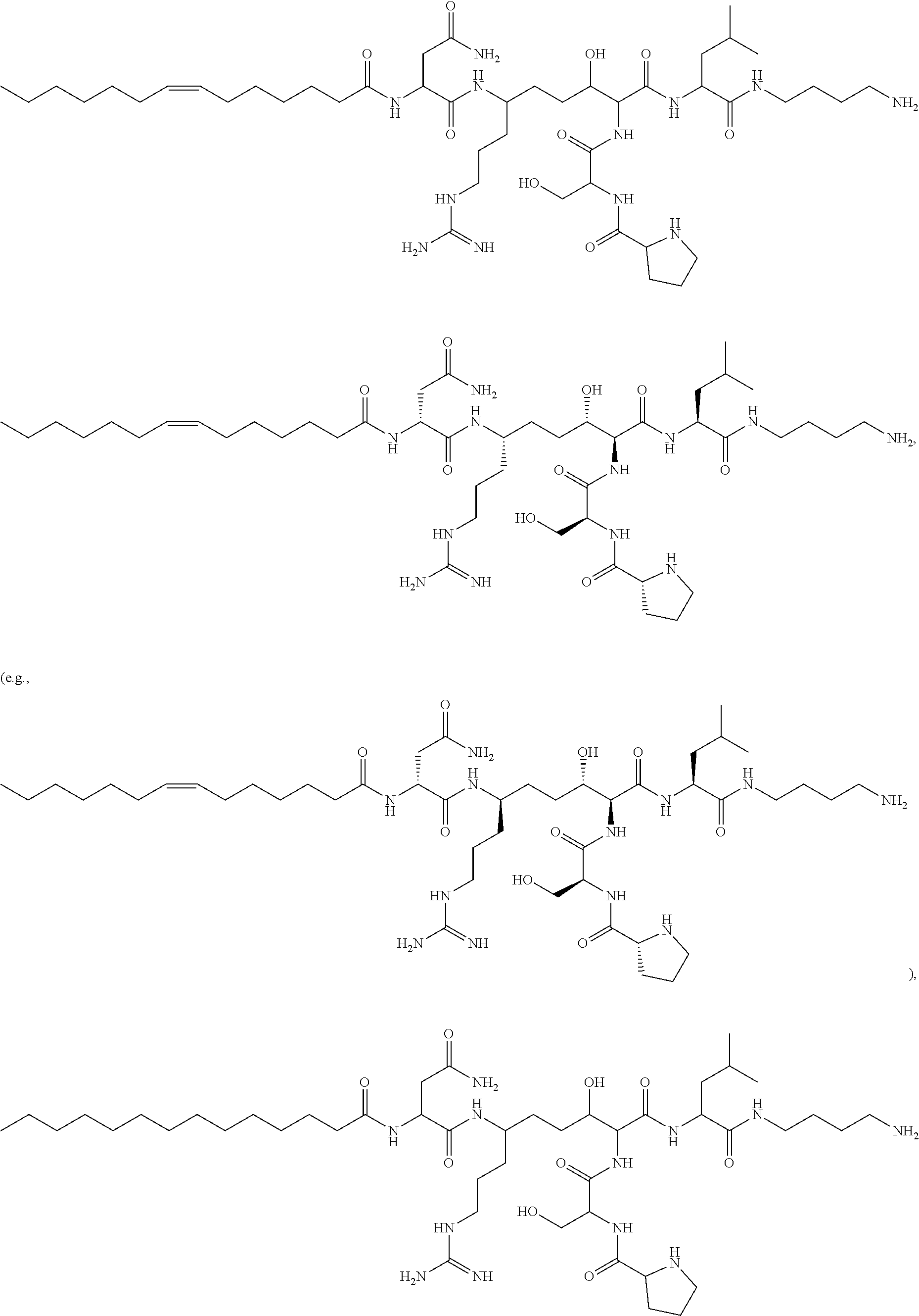

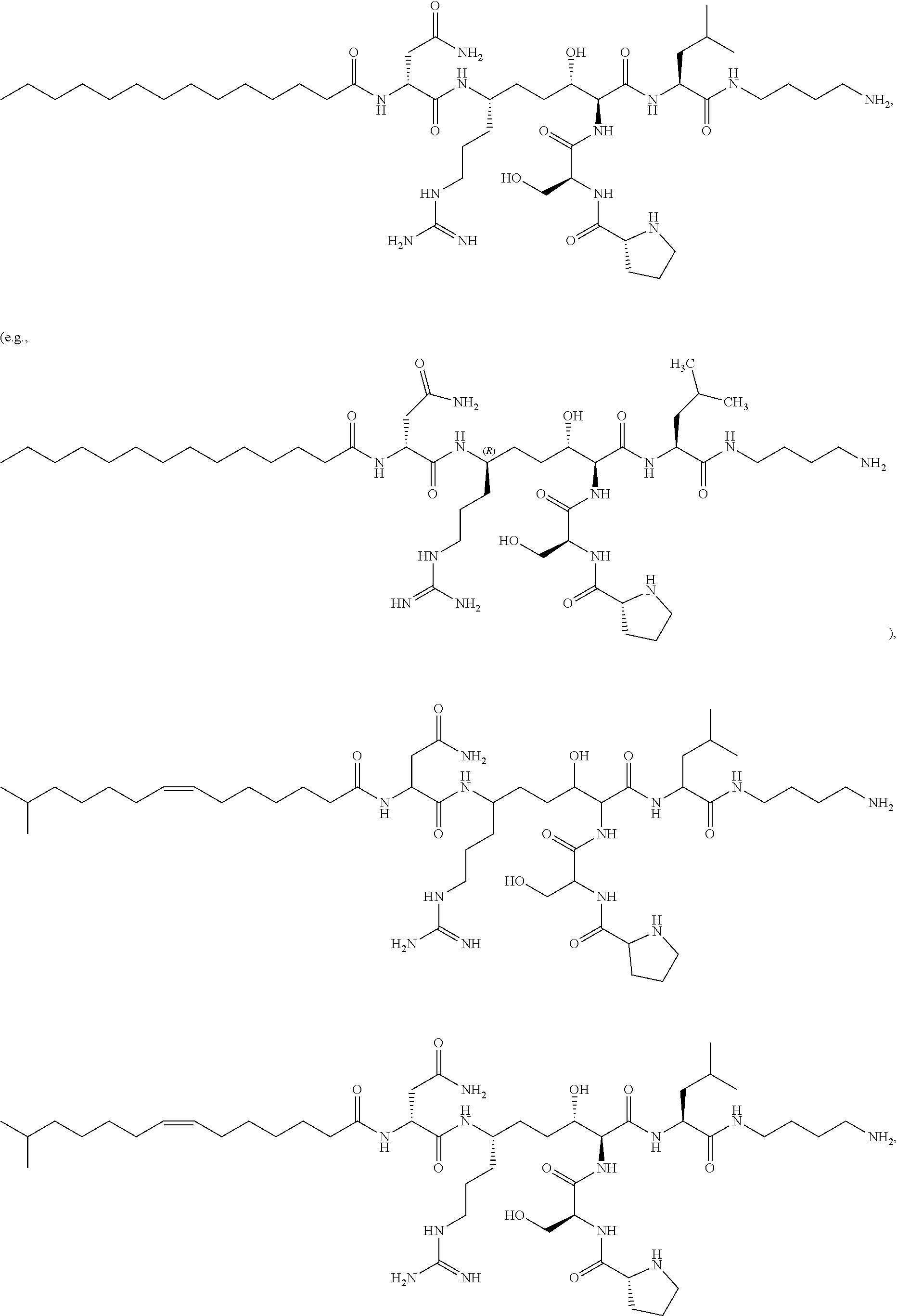

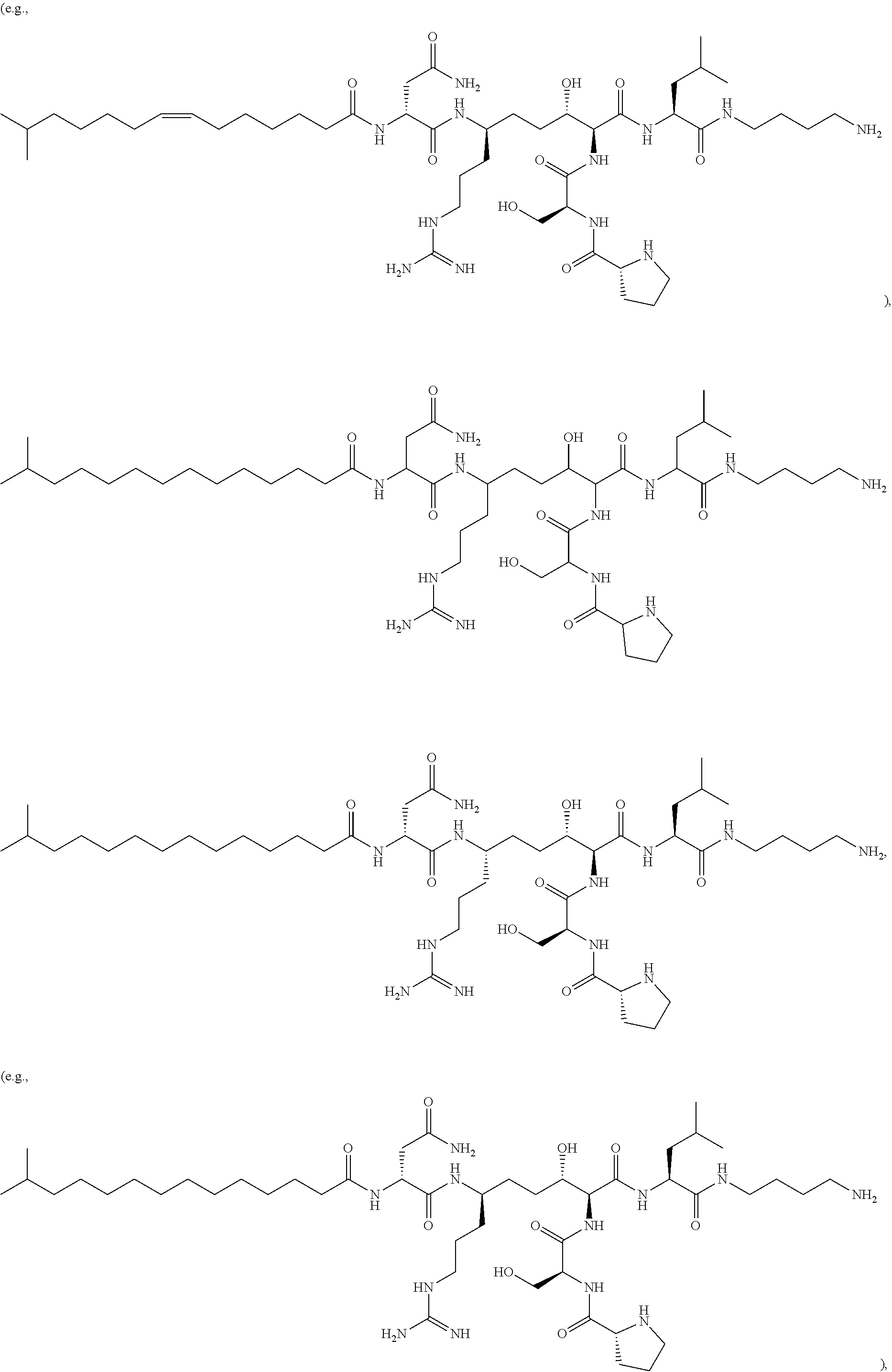

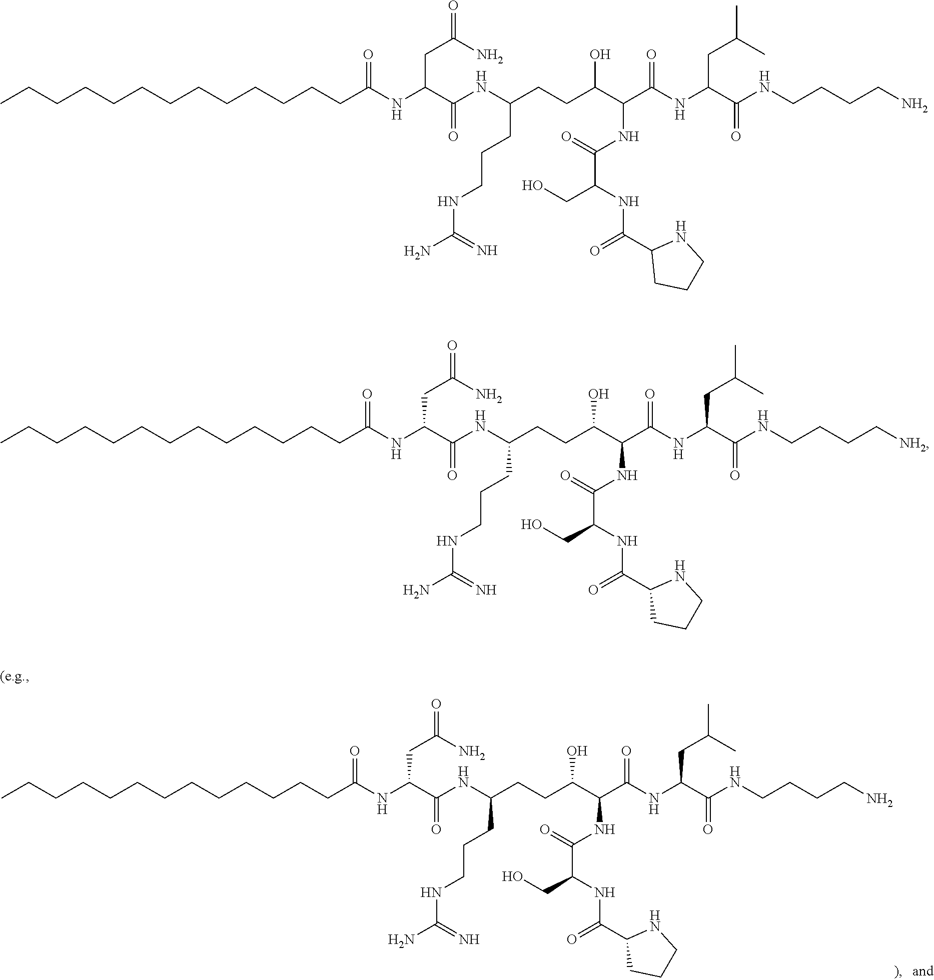 [SMILES: CCCCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CCCCCCCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CCCCCCCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]

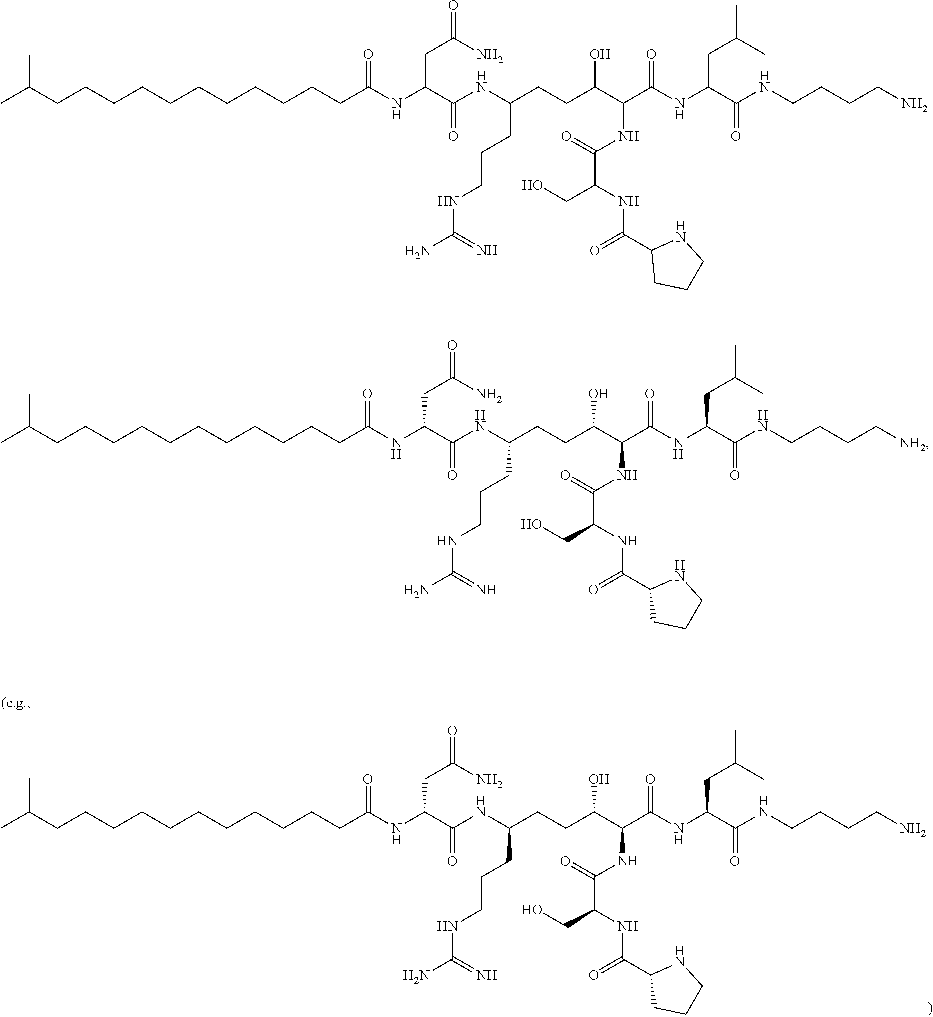 [SMILES: CC(C)CCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CC(C)CCCCCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CC(C)CCCCCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]

can be converted in vivo or in vitro to the following compound:

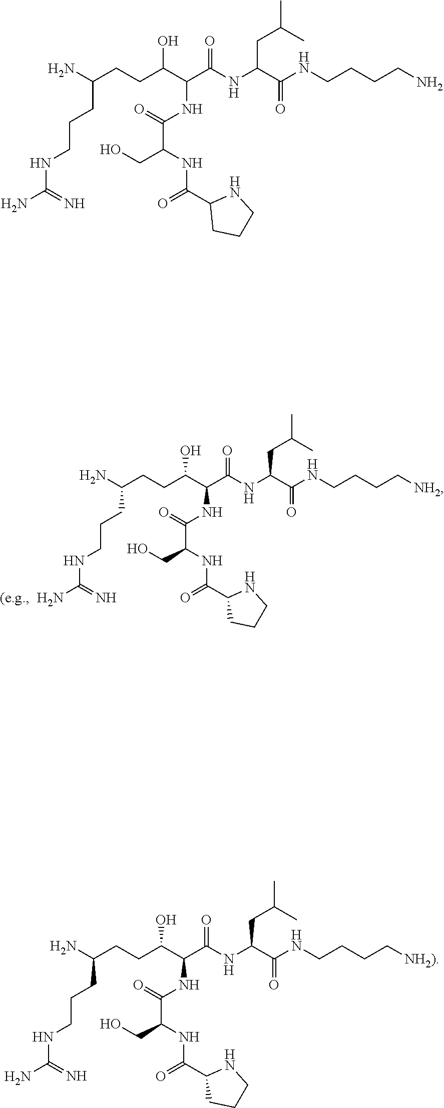

In various examples, the following compounds (e.g., prodrug antibiotics):

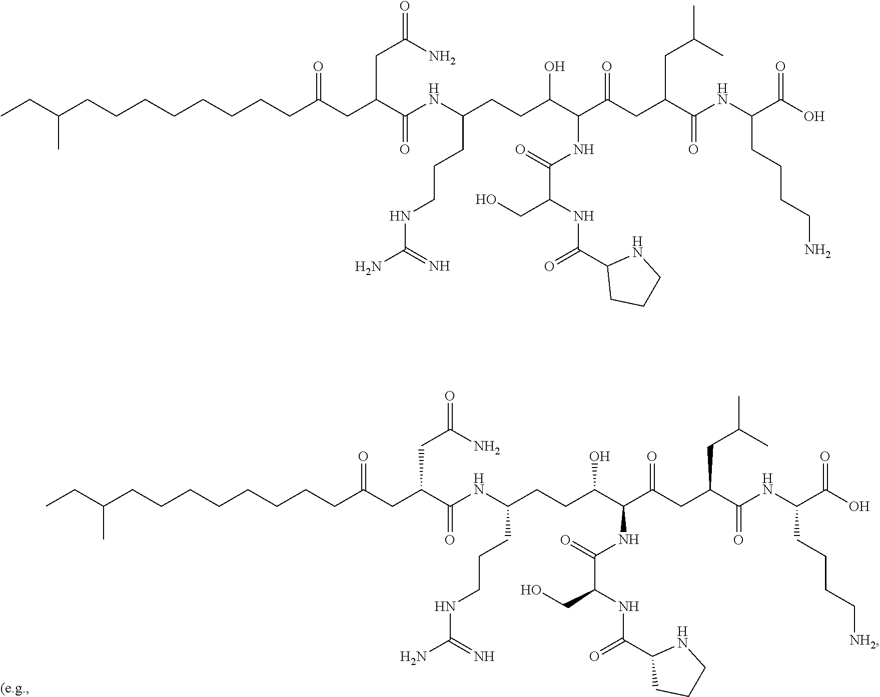 [SMILES: CCC(C)CCCCCCCCC(=O)CC(CC(N)=O)C(=O)NC(CCCNC(=N)N)CCC(O)C(NC(=O)C(CO)NC(=O)C1CCCN1)C(=O)CC(CC(C)C)C(=O)NC(CCCCN)C(=O)O ; CCC(C)CCCCCCCCC(=O)C[C@H](CC(N)=O)C(=O)N[C@@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCN)C(=O)O]

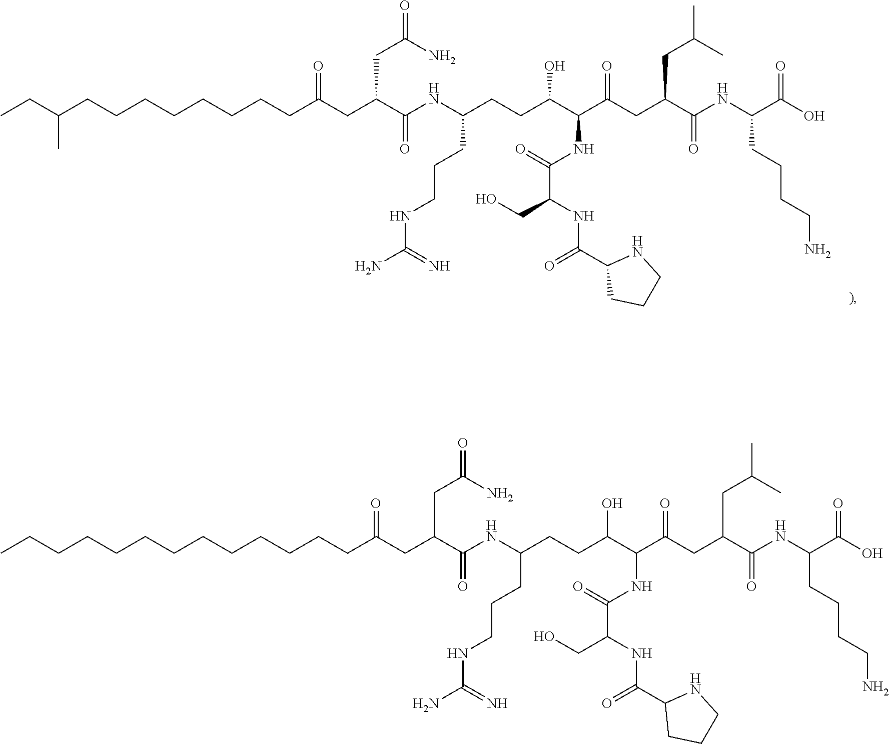 [SMILES: CCC(C)CCCCCCCCC(=O)C[C@H](CC(N)=O)C(=O)N[C@@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCN)C(=O)O ; CCCCCCCCCCCCCC(=O)CC(CC(N)=O)C(=O)NC(CCCNC(=N)N)CCC(O)C(NC(=O)C(CO)NC(=O)C1CCCN1)C(=O)CC(CC(C)C)C(=O)NC(CCCCN)C(=O)O]

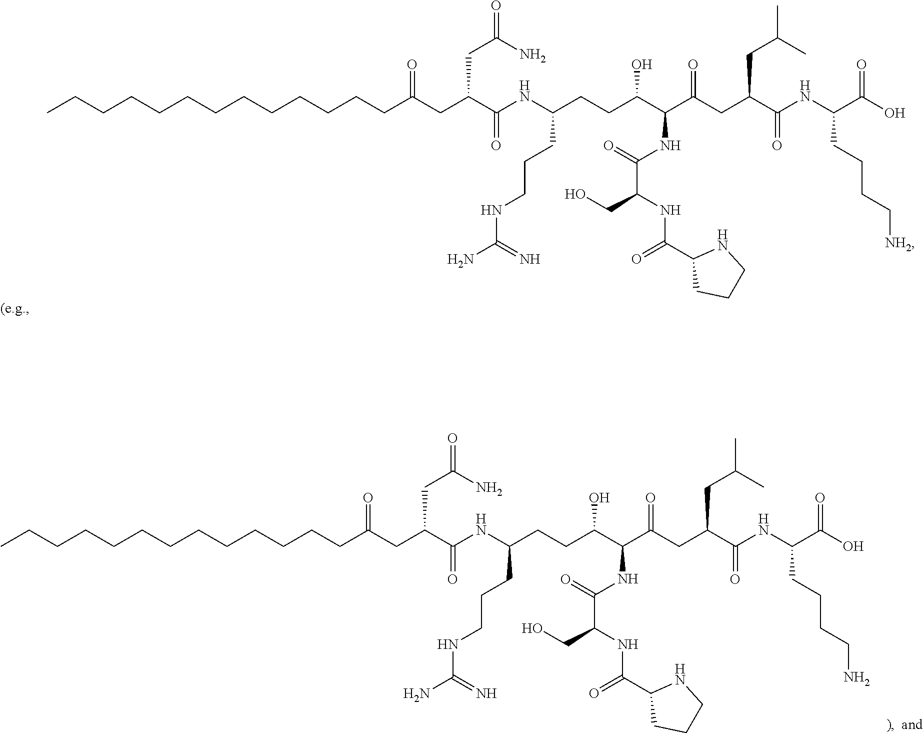 [SMILES: CCCCCCCCCCCCCC(=O)C[C@H](CC(N)=O)C(=O)N[C@@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCN)C(=O)O ; CCCCCCCCCCCCCC(=O)C[C@H](CC(N)=O)C(=O)N[C@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCN)C(=O)O]

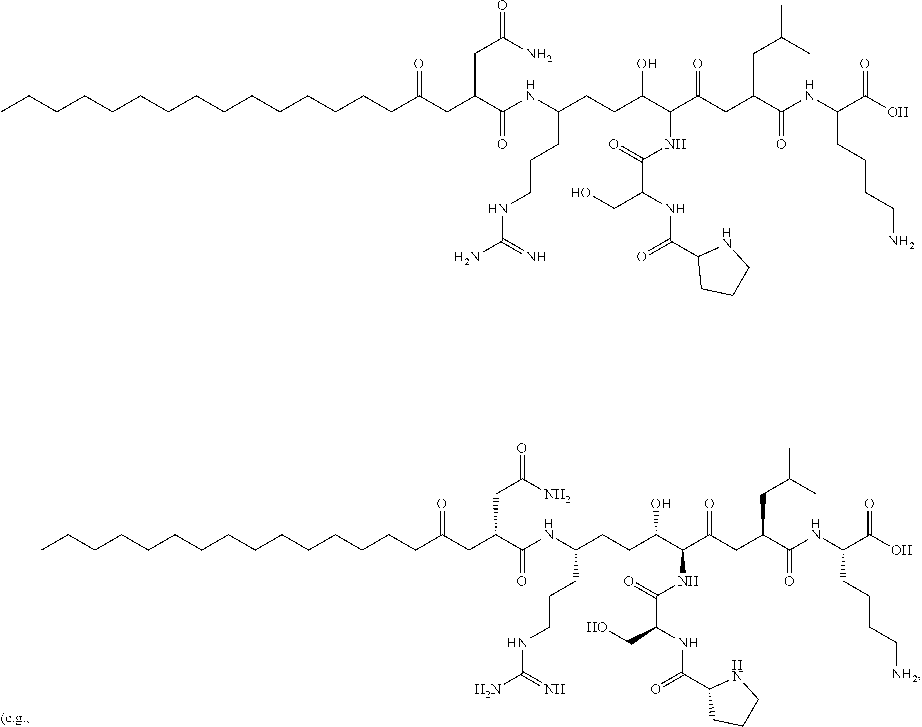 [SMILES: CCCCCCCCCCCCCCCC(=O)CC(CC(N)=O)C(=O)NC(CCCNC(=N)N)CCC(O)C(NC(=O)C(CO)NC(=O)C1CCCN1)C(=O)CC(CC(C)C)C(=O)NC(CCCCN)C(=O)O ; CCCCCCCCCCCCCCCC(=O)C[C@H](CC(N)=O)C(=O)N[C@@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCN)C(=O)O]

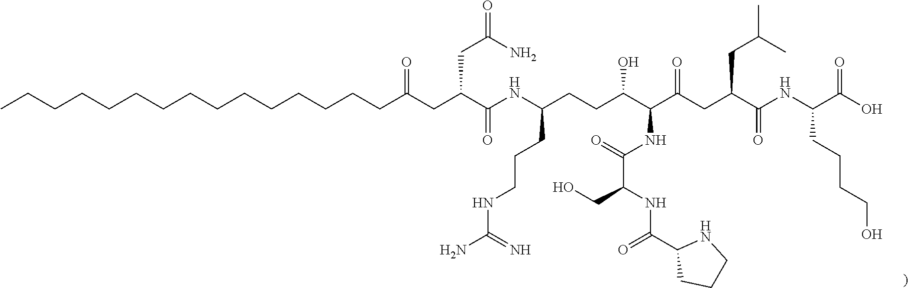 [SMILES: CCCCCCCCCCCCCCCC(=O)C[C@H](CC(N)=O)C(=O)N[C@H](CCCNC(=N)N)CC[C@H](O)[C@H](NC(=O)[C@H](CO)NC(=O)[C@H]1CCCN1)C(=O)C[C@@H](CC(C)C)C(=O)N[C@@H](CCCCO)C(=O)O]

can be converted in vivo or in vitro to the following compound:

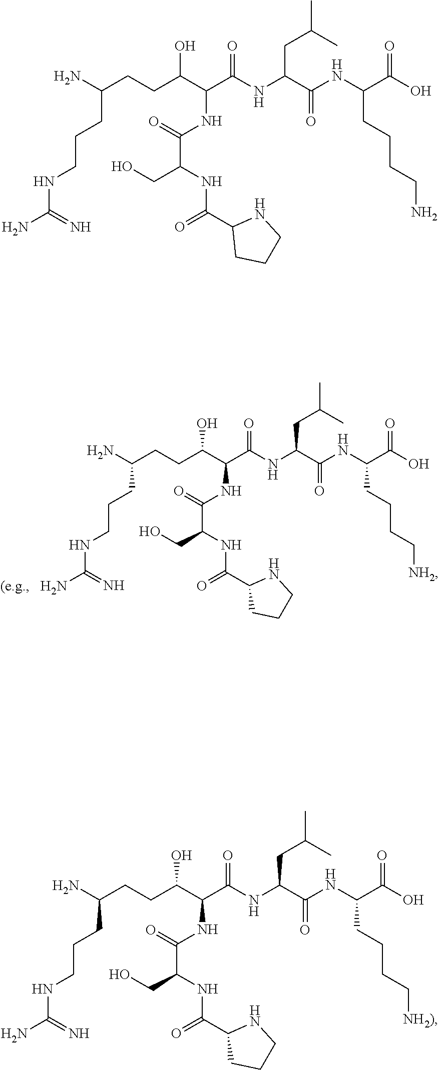

In various examples, the following compounds (e.g., prodrug antibiotics):

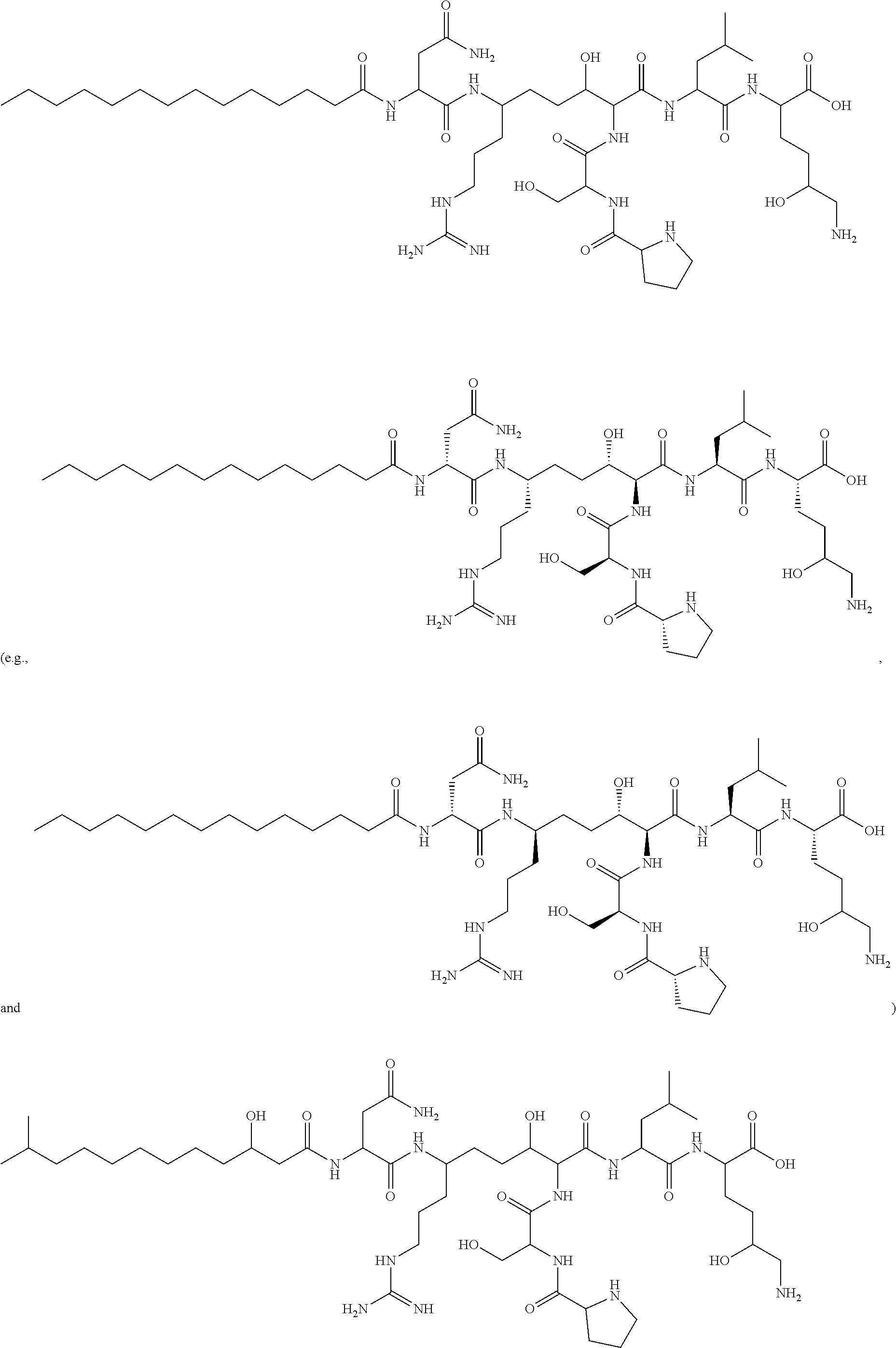

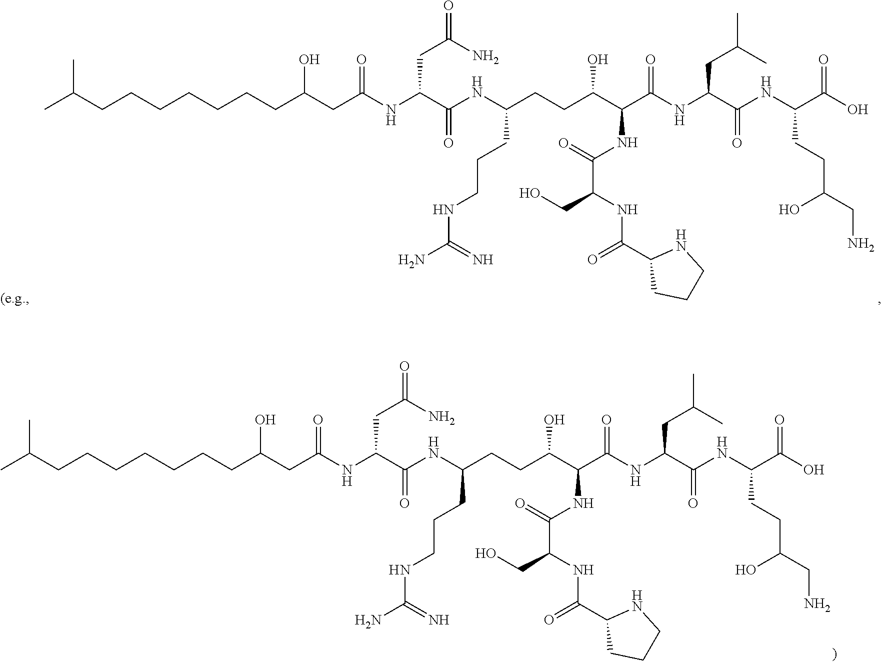 [SMILES: CC(C)CCCCCCCC(O)CC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)N[C@@H](CCC(O)CN)C(=O)O ; CC(C)CCCCCCCC(O)CC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)N[C@@H](CCC(O)CN)C(=O)O]

can be converted in vivo or in vitro to the following compound:

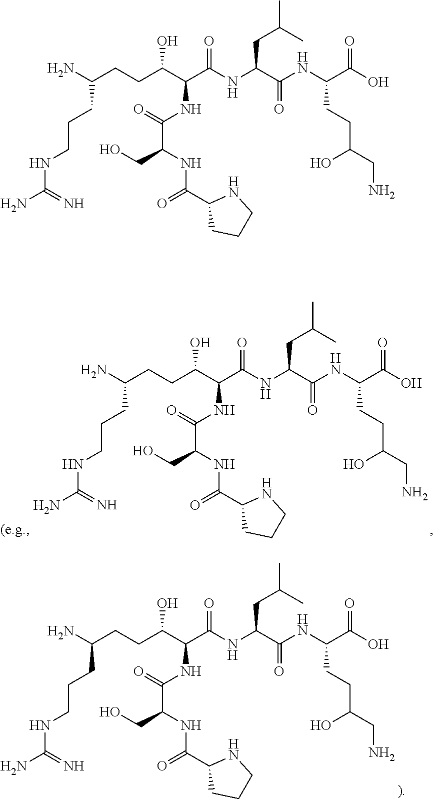

In various examples, the following compound (e.g., prodrug antibiotic):

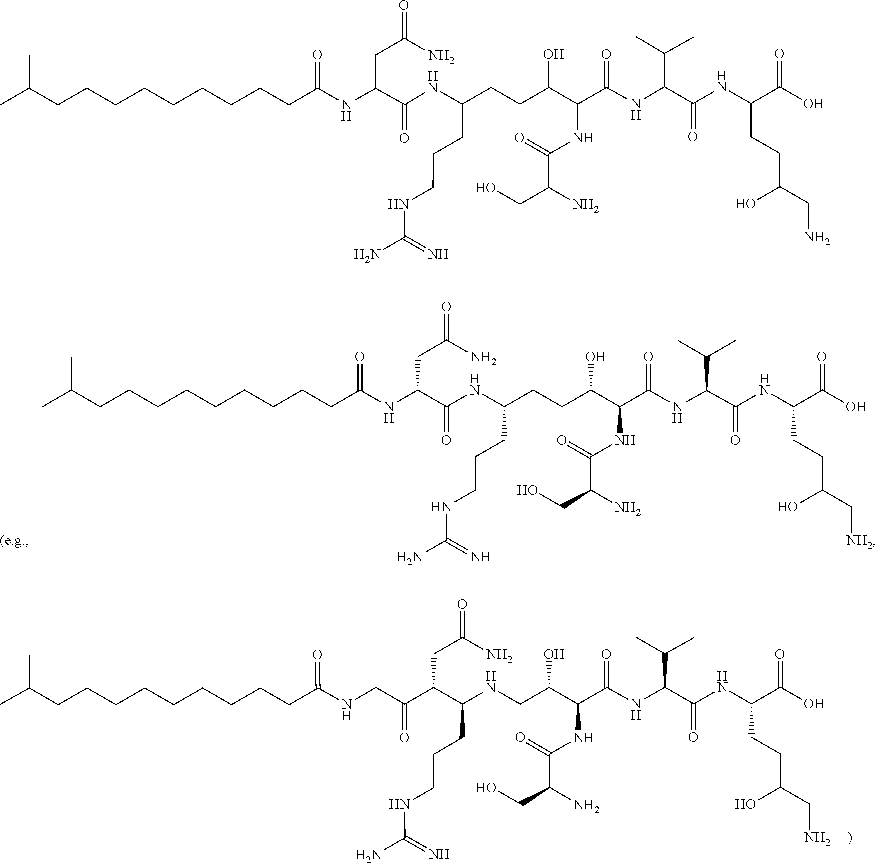

can be converted in vivo or in vitro to the following compound:

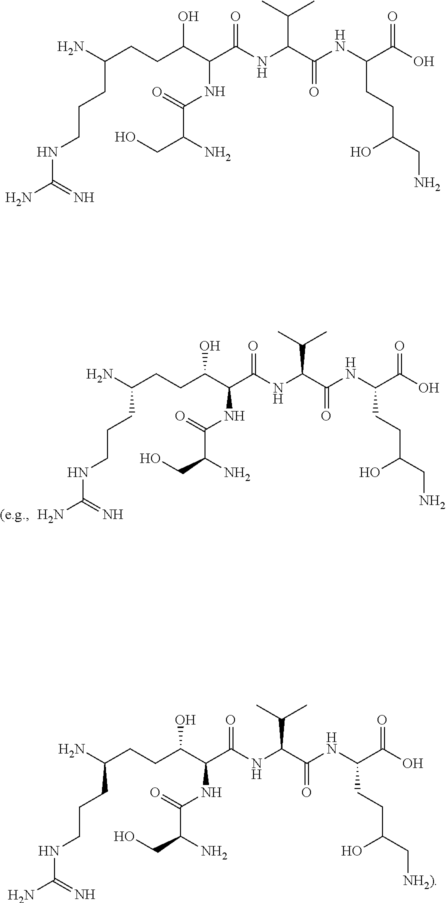

In various examples, the following compounds (e.g., prodrug antibiotics):

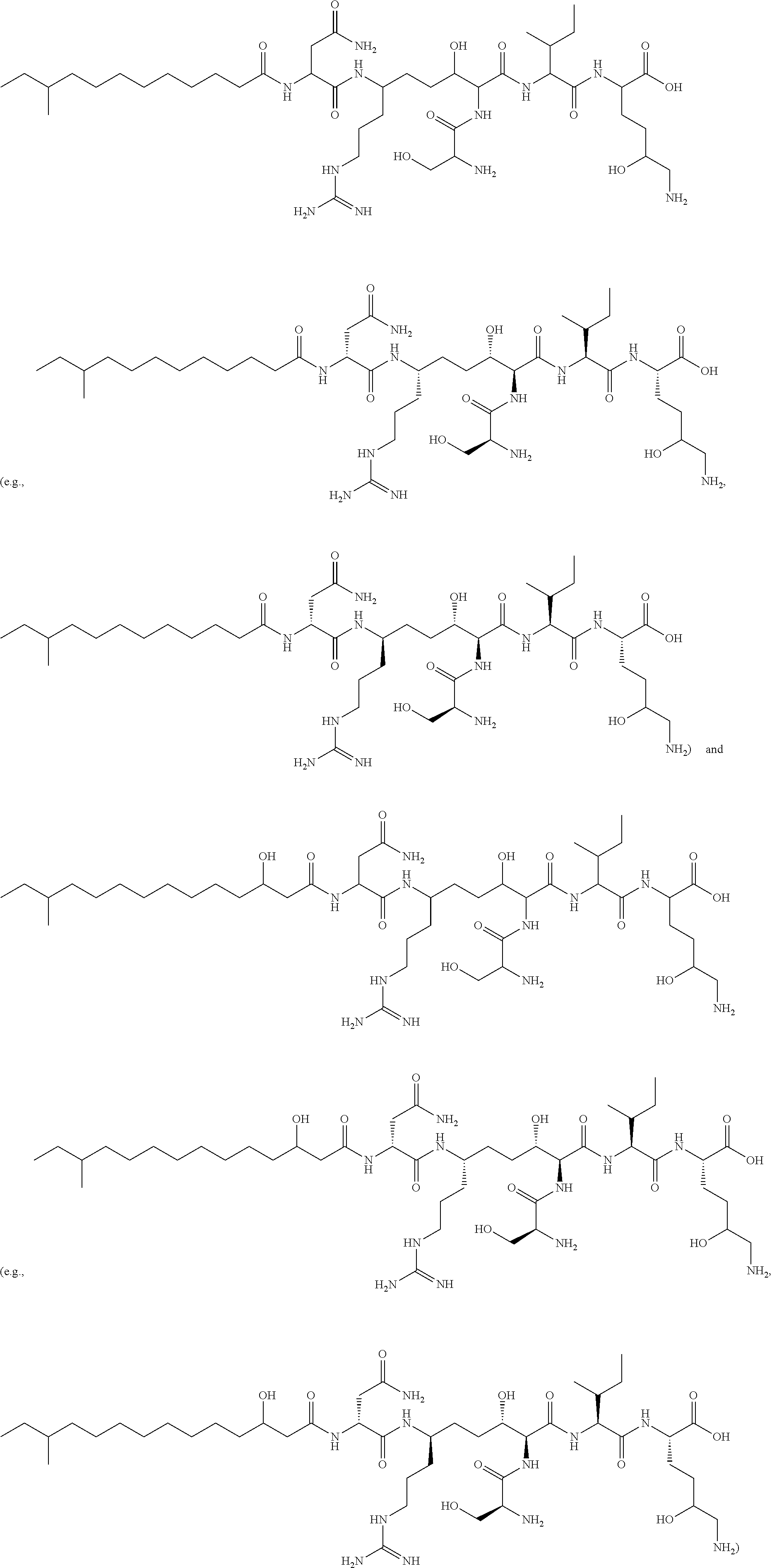

can be converted in vivo or in vitro to the following compound:

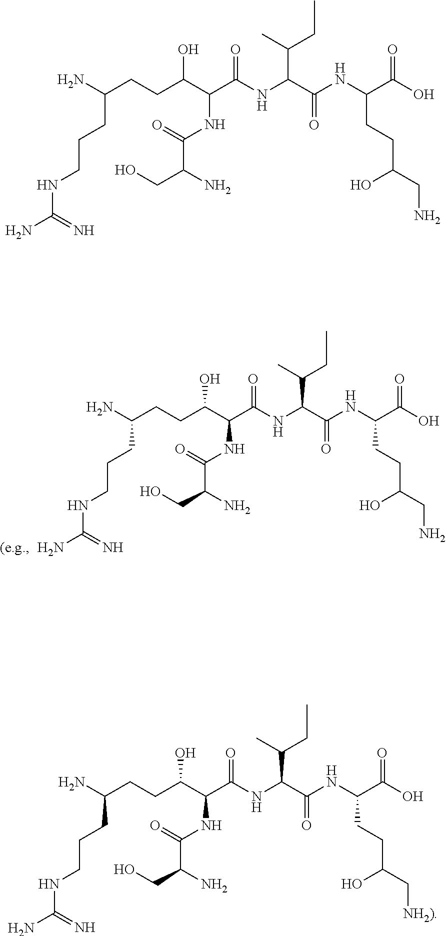

In various examples, the following compounds (e.g., prodrug antibiotics):

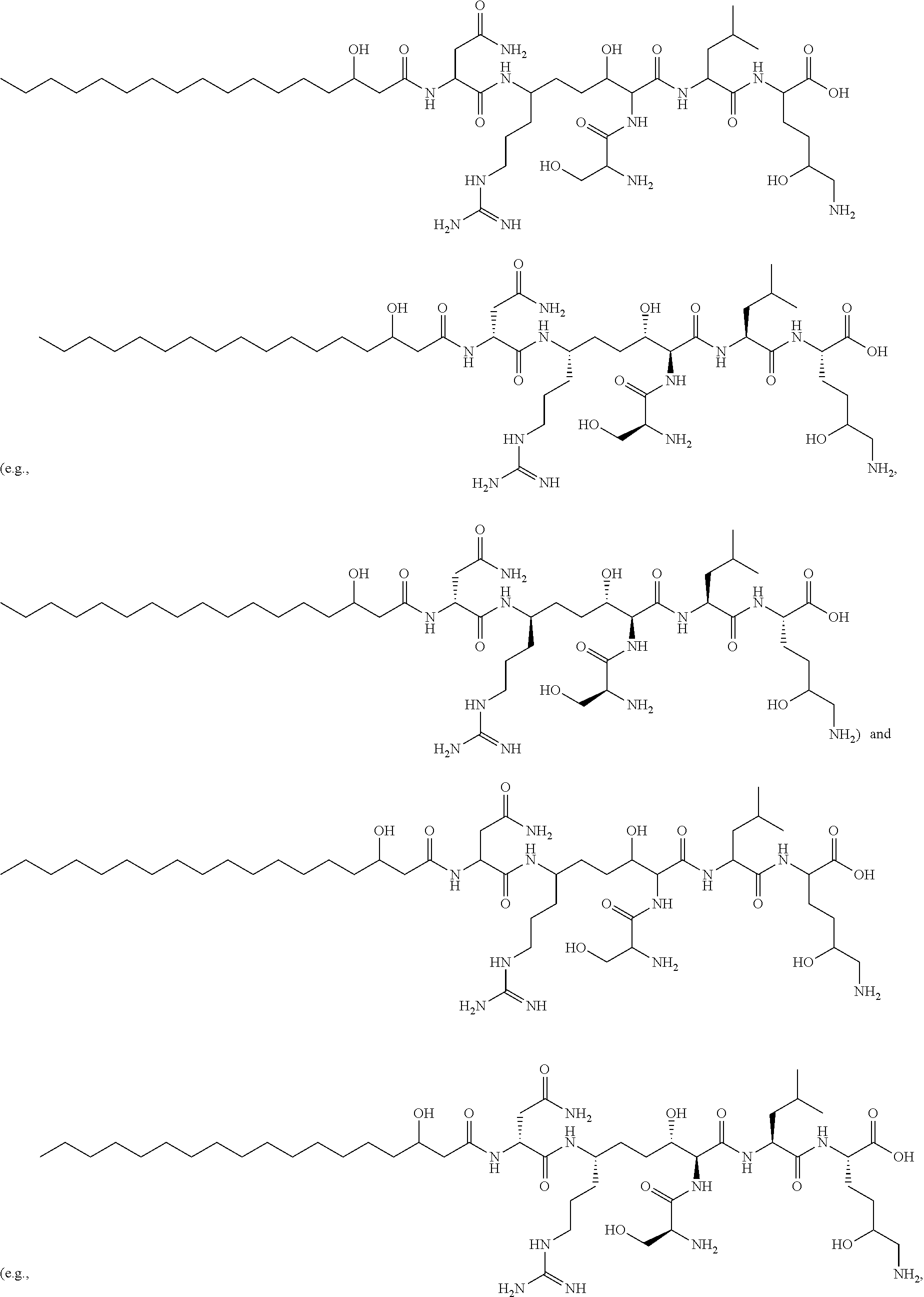

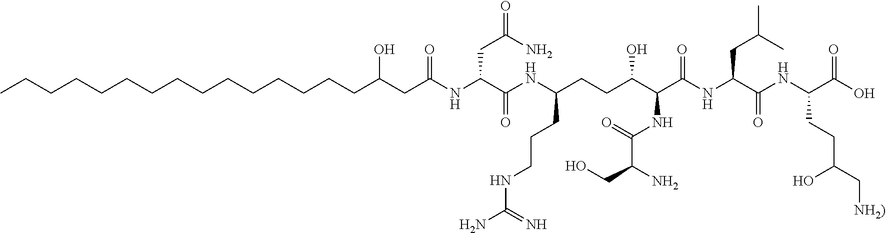

can be converted in vivo or in vitro to the following compound:

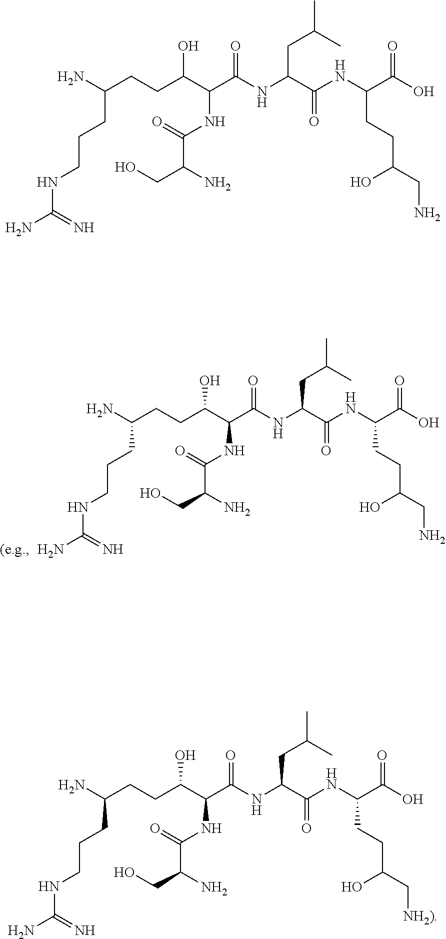

In various examples, the method may be performed in vivo in a subject in need of treatment. The subject to be treated by the method of the disclosure may be human or non-human (e.g., mammal). Non-human animals include ungulates such as bovines. Additional on-limiting examples of non-human mammals include pigs, mice, rats, rabbits, cats, dogs, or other agricultural mammals, pet, or service animals, and the like.

In various examples, the subject in need of treatment may be genetically predisposed to colorectal cancer and/or the subject has colitis and/or irritable bowel syndrome (IBS) and/or an inflammatory bowel disorder. In various examples, the administration may be for the purpose of prophylaxis. In various other examples, the administration may be to partially or completely cure an individual having colorectal cancer and/or the subject has colitis and/or irritable bowel syndrome (IBS) and/or an inflammatory bowel disorder.

In various examples, a prodrug antibiotic of the present disclosure may be used in the treatment of colorectal cancer in combination with other forms of treatment. For example, the other form of treatment may be radiation and/or chemotherapy and/or other known cancer treatment regimes. Various chemotherapy agents (e.g., one or more different chemotherapy agents) may be used in combination with a prodrug antibiotic of the present disclosure.

Various chemotherapy agents (e.g., chemotherapy drugs) can be used. Any FDA approved chemotherapy agents (e.g., chemotherapy drugs) can be used. Combinations of chemotherapy agents can be used. Non-limiting examples of chemotherapy agents and combinations include abemaciclib, abiraterone acetate, ABITREXATE® (methotrexate), ABVD (doxorubicin, bleomycin, vinblastine, and dacarbazine), ABVE (doxorubicin, bleomycin, vincristine sulfate, etoposide phosphate), ABVE-PC (doxorubicin, bleomycin, vincristine sulfate, etoposide phosphate, prednisone, cyclophosphamide), AC (doxorubicin and cyclophosphamide), acalabrutinib, AC-T (doxorubicin, cyclophosphamide, paclitaxel), ADE (cytarabine, daunorubicin, etoposide), ADRIAMYCIN® (doxorubicin hydrochloride), afatinib dimaleate, AFINITOR® (everolimus), AKYNZEO® (netupitant and palonosetron hydrochloride), ALDARA® (imiquimod), aldesleukin, ALECENSA® (alectinib), alectinib, ALIMTA® (pemetrexed disodium), ALIQOPA® (copanlisib hydrochloride), ALKERAN® for injection (melphalan hydrochloride), ALKERAN® tablets (melphalan), ALOXI® (palonosetron hydrochloride), ALUNBRIG™ (brigatinib), ambochlorin (chlorambucil), amboclorin (chlorambucil), amifostine, aminolevulinic acid, anastrozole, aprepitant, AREDIA® (pamidronate disodium), ARIIVDEX® (anastrozole), AROMASIN® (exemestane), ARRANON® (nelarabine), arsenic trioxide, asparaginase Erwinia chrysanthemi, axicabtagene ciloleucel, axitinib, azacitidine, BEACOPP (bleomycin, etoposide, doxorubicin, cyclophosphamide, vincristine, procarbazine, prednisone), Becenum® (carmustine), Beleodaq® (belinostat), belinostat, bendamustine hydrochloride, BEP (bleomycin, etoposide, cisplatin), bexarotene, bicalutamide, BICNU® (carmustine), bleomycin, bortezomib, Bosulif® (bosutinib), bosutinib, brigatinib, BuMel (busulfan, melphalan hydrochloride), busulfan, BUSULFEX® (busulfan), cabazitaxel, CABOMETYX™ (cabozantinib-S-malate), cabozantinib-S-malate, CAF (cyclophosphamide, doxorubicin, 5-fluorouracil), CALQUENCE® (acalabrutinib), CAMPTOSAR® (irinotecan hydrochloride), capecitabine, CAPOX, CARAC® (fluorouracil-topical), carboplatin, carboplatin-TAXOL®, carfilzomib, carmubris (carmustine), carmustine, carmustine implant, CASODEX® (bicalutamide), CEM (carboplatin, etoposide, melphalan), ceritinib, CERUBIDINE® (daunorubicin hydrochloride), CEV (carboplatin, etoposide phosphate, vincristine sulfate), chlorambucil, chlorambucil-prednisone, CHOP (cyclophosphamide, doxorubicin, vincristine, prednisone), cisplatin, cladribine, CLAFEN® (cyclophosphamide), clofarabine, CLOFAREX® (clofarabine), CLOLAR® (clofarabine), CMF (cyclophosphamide, methotrexate, fluorouracil), cobimetinib, COMETRIQ® (cabozantinib-S-malate), copanlisib hydrochloride, COPDAC (cyclophosphamide, vincristine sulfate, prednisone, dacarbazine), COPP (cyclophosphamide, vincristine, procarbazine, prednisone), COPP-ABV (cyclophosphamide, vincristine, procarbazine, prednisone, doxorubicin, bleomycin, vinblastine sulfate), COSMEGEN® (dactinomycin), COTELLIC® (cobimetinib), crizotinib, CVP (cyclophosphamide, vincristine, prednisolone), cyclophosphamide, CYFOS® (ifosfamide), cytarabine, cytarabine liposome, CYTOSAR-U® (cytarabine), CYTOXAN® (cyclophosphamide), dabrafenib, dacarbazine, DACOGEN® (decitabine), dactinomycin, dasatinib, daunorubicin hydrochloride, daunorubicin hydrochloride and cytarabine liposome, decitabine, defibrotide sodium, DEFITELIO® (defibrotide sodium), degarelix, denileukin diftitox, dexamethasone, dexrazoxane hydrochloride, docetaxel, doxorubicin, doxorubicin hydrochloride, doxorubicin hydrochloride liposome, DOX-SL® (doxorubicin hydrochloride liposome), DTIC-DOME® (dacarbazine), ELITEK® (rasburicase), ELLENCE® (epirubicin hydrochloride), ELOXATIN® (oxaliplatin), eltrombopag olamine, EMEND® (aprepitant), enasidenib mesylate, enzalutamide, epirubicin hydrochloride, EPOCH (etoposide, prednisone, vincristine, cyclophosphamide, and doxorubicin hydrochloride), eribulin mesylate, ERIVEDGE® (vismodegib), erlotinib hydrochloride, ERWINAZE® (asparaginase Erwinia chrysanthemi), ETHYOL® (amifostine), ETOPOPHOS® (etoposide phosphate), etoposide, etoposide phosphate, everolimus, EVISTA® (raloxifene hydrochloride), EVOMELA® (melphalan hydrochloride), exemestane, 5-FU (fluorouracil), FARESTON® (toremifene), FARYDAK® (panobinostat), FASLODEX® (fulvestrant), FEC (5-fluorouracil, epirubicin, cyclophosphamide), FEMARA® (letrozole), filgrastim, FLUDARA® (fludarabine phosphate), fludarabine phosphate, flutamide, FOLEX® (methotrexate), FOLEX PFS® (methotrexate), FOLFIRI (leucovorin calcium, fluorouracil, irinotecan hydrochloride), FOLFIRINOX (leucovorin calcium, fluorouracil, irinotecan hydrochloride, oxaliplatin), FOLFOX (leucovorin calcium, fluorouracil, oxaliplatin), FOLOTYN® (pralatrexate), FU-LV (fluorouracil, leucovorin calcium), fulvestrant, gefitinib, gemcitabine hydrochloride, gemcitabine-cisplatin, gemcitabine-oxaliplatin, GEMZAR® (gemcitabine hydrochloride), GILOTRIF® (afatinib dimaleate), GLEEVEC® (imatinib mesylate), GLIADEL® (carmustine implant), goserelin acetate, HALAVEN® (eribulin mesylate), HEMANGEOL® (propranolol hydrochloride), Hycamtin® (topotecan hydrochloride), HYDREA® (hydroxyurea), hydroxyurea, Hyper-CVAD (course A: cyclophosphamide, vincristine, doxorubicin, dexamethasone, cytarabine, mesna, methotrexate; and course B: methotrexate, leucovorin, sodium bicarbonate, cytarabine), IBRANCE® (palbociclib), ibrutinib, ICE (ifosfamide, mesna, carboplatin, etoposide), ICLUSIG® (ponatinib hydrochloride), IDAMYCIN® (idarubicin hydrochloride), idarubicin hydrochloride, idelalisib, IDHIFA® (enasidenib mesylate), IFEX® (ifosfamide), ifosfamide, IFOSFAMIDUM™ (ifosfamide), imatinib mesylate, IMBRUVICA® (ibrutinib), imiquimod, IMLYGIC® (talimogene laherparepvec), INLYTA® (axitinib), IRESSA® (gefitinib), irinotecan, irinotecan hydrochloride, irinotecan hydrochloride liposome, ISTODAX® (romidepsin), ixabepilone, ixazomib citrate, IXEMPRA® (ixabepilone), JAKAFI® (ruxolitinib phosphate), JEB (carboplatin, etoposide phosphate, bleomycin), JEVTANA® (cabazitaxel), KEOXIFENE™ (raloxifene hydrochloride), KEPIVANCE® (palifermin), KISQALI® (ribociclib), KYMRIAH™ (tisagenlecleucel), KYPROLIS® (carfilzomib), lanreotide acetate, lapatinib ditosylate, lenalidomide, lenvatinib mesylate, LENVIMA® (lenvatinib mesylate), letrozole, leucovorin calcium, LEUKERAN® (chlorambucil), leuprolide acetate, LEUSTATIN® (cladribine), LEVULAN® (aminolevulinic acid), LINFOLIZIN™ (chlorambucil), lomustine, LONSURF® (trifluridine and tipiracil hydrochloride), LUPRON® (leuprolide acetate), LUPRON DEPOT® (leuprolide acetate), LUPRON DEPOT-PED® (leuprolide acetate), LYNPARZA® (olaparib), MATULANE® (procarbazine hydrochloride), mechlorethamine hydrochloride, megestrol acetate, MEKINIST® (trametinib), melphalan, melphalan hydrochloride, mercaptopurine, mesna, MESNEX® (Mesna), METHAZOLASTONE™ (temozolomide), methotrexate, METHOTREXATE LPF™ (methotrexate), methylnaltrexone bromide, MEXATE® (methotrexate), MEXATE-AQ™ (methotrexate), midostaurin, mitomycin C, mitoxantrone hydrochloride, MITOZYTREX™ (mitomycin C), MOPP (mustargen, vincristine, procarbazine, prednisone), MOZOBIL™ (plerixafor), MUSTARGEN® (mechlorethamine hydrochloride), MUTAMYCINT™ (mitomycin C), MYLERAN® (busulfan), MYLOSAR® (azacitidine), NAVELBINE® (vinorelbine tartrate), nelarabine, NEOSAR® (cyclophosphamide), neratinib maleate, NERLYNX® (neratinib maleate), netupitant and palonosetron hydrochloride, NEULASTA® (pegfilgrastim), NEUPOGEN® (filgrastim), NEXAVAR® (sorafenib tosylate), NILANDRON® (nilutamide), nilotinib, nilutamide, NINLARO® (ixazomib citrate), niraparib tosylate monohydrate, NOLVADEX® (tamoxifen citrate), NPLATE® (romiplostim), ODOMZO® (sonidegib), OEPA (vincristine sulfate, etoposide phosphate, prednisone, doxorubicin hydrochloride), OFF (oxaliplatin, fluorouracil, leucovorin), olaparib, omacetaxine mepesuccinate, ondansetron hydrochloride, ONTAK® (denileukin diftitox), OPPA (vincristine sulfate, procarbazine hydrochloride, prednisone, doxorubicin hydrochloride), osimertinib, oxaliplatin, paclitaxel, PAD (bortezomib, doxorubicin hydrochloride, dexamethasone), palbociclib, palifermin, palonosetron hydrochloride, pamidronate disodium, panobinostat, paraplat (carboplatin), PARAPLATIN® (carboplatin), pazopanib hydrochloride, PCV (procarbazine hydrochloride, lomustine, vincristine sulfate), PEB (cisplatin, etoposide phosphate, bleomycin), pegfilgrastim, pemetrexed disodium, PLATINOL® (cisplatin), PLATINOL®-AQ (cisplatin), plerixafor, pomalidomide, POMALYST® (pomalidomide), ponatinib hydrochloride, pralatrexate, prednisone, procarbazine hydrochloride, PROMACTA® (eltrombopag olamine), propranolol hydrochloride, PURINETHOL® (mercaptopurine), PURIXAN® (mercaptopurine), radium 223 dichloride, raloxifene hydrochloride, rasburicase, regorafenib, RELISTOR® (methylnaltrexone bromide), REVLIMID® (lenalidomide), RHEUMATREX® (methotrexate), ribociclib, rolapitant hydrochloride, romidepsin, romiplostim, rubidomycin (daunorubicin hydrochloride), RUBRACA® (rucaparib camsylate), rucaparib camsylate, ruxolitinib phosphate, RYDAPT® (midostaurin), SCLEROSOL® Intrapleural Aerosol (Talc), sipuleucel-T, SOMATULINE® Depot (lanreotide acetate), sonidegib, sorafenib tosylate, SPRYCEL® (dasatinib), Stanford V (mechlorethamine hydrochloride, doxorubicin hydrochloride, vinblastine sulfate, vincristine sulfate, bleomycin, etoposide phosphate, prednisone), sterile talc powder (Talc), STERITALC® (Talc), STIVARGA® (regorafenib), sunitinib malate, SUTENT® (sunitinib malate), SYNRIBO™ (omacetaxine mepesuccinate), TABLOID® (thioguanine), TAC (docetaxel, doxorubicin hydrochloride, cyclophosphamide), TAFINLAR® (dabrafenib), TAGRISSO® (osimertinib), Talc, tamoxifen citrate, TARABINE PFS® (cytarabine), TARCEVA® (erlotinib hydrochloride), TARGRETIN® (bexarotene), TASIGNA® (nilotinib), TAXOL® (Paclitaxel), TAXOTERE® (docetaxel), TEMODAR® (temozolomide), temozolomide, temsirolimus, thalidomide, THALOMID® (thalidomide), thioguanine, thiotepa, TOTECT® (dexrazoxane hydrochloride), TPF (docetaxel, cisplatin, fluorouracil), trabectedin, trametinib, TREANDA® (bendamustine hydrochloride), trifluridine and tipiracil hydrochloride, TRISENOX® (arsenic trioxide), TYKERB® (lapatinib ditosylate), uridine triacetate, VAC (vincristine sulfate, dactinomycin, cyclophosphamide), valrubicin, VALSTAR® (valrubicin), vandetanib, VAMP (vincristine sulfate, doxorubicin hydrochloride, methotrexate, prednisone), VARUBI® (rolapitant hydrochloride), VeIP (vinblastine sulfate, ifosfamide, cisplatin), VELBAN® (vinblastine sulfate), VELCADE® (bortezomib), VELSAR® (vinblastine sulfate), vemurafenib, VENCLEXTA™ (venetoclax), venetoclax, VERZENIO™ (abemaciclib), VIADUR® (leuprolide acetate), VIDAZA® (azacitidine), vinblastine sulfate, VINCASAR PFS® (vincristine sulfate), vincristine sulfate, vinorelbine tartrate, VIP (etoposide phosphate, ifosfamide, cisplatin), vismodegib, VISTOGARD® (uridine triacetate), vorinostat, VOTRIENT® (pazopanib hydrochloride), WELLCOVORIN® (leucovorin calcium), XALKORI® (crizotinib), XELODA® (capecitabine), XELIRI (capecitabine, irinotecan hydrochloride), XELOX (capecitabine, oxaliplatin), XOFIGO® (radium 223 dichloride), XTANDI® (enzalutamide), YESCARTA™ (axicabtagene ciloleucel), YONDELIS® (trabectedin), ZALTRAP® (ziv-aflibercept), ZARXIO® (filgrastim), ZEJULA® (niraparib tosylate monohydrate), ZELBORAF® (vemurafenib), ZINECARD® (dexrazoxane hydrochloride), ZOFRAN® (ondansetron hydrochloride), ZOLADEX® (goserelin acetate), zoledronic acid, ZOLINZA® (vorinostat), ZOMETA® (zoledronic acid), ZYDELIG® (idelalisib), ZYKADIA® (ceritinib), and ZYTIGA® (abiraterone acetate).

In various examples, the chemotherapy drug or agent is doxorubicin, cisplatin, carboplatin, pemetrexed, auristatin, maytansine, paclitaxel, camptothecin, vincristine, vinblastine, irinotecan, amphotericin B, salts thereof, or combinations thereof.

Compositions comprising a compound of the disclosure and a pharmaceutical agent for administration can be prepared at a patient's bedside, or by a pharmaceutical manufacturer. In the latter case, the compositions can be provided in any suitable container, such as a sealed sterile vial or ampoule, and may be further packaged to include instruction documents for use by a pharmacist, physician, or other health care provider. The compositions can be provided as a liquid, or as a lyophilized or powder form that can be reconstituted, if necessary, when ready for use. In particular, the compositions can be provided in combination with any suitable delivery form or vehicle, examples of which include, for example, liquids, caplets, capsules, tablets, inhalants, or aerosol, etc. The delivery devices may comprise components that facilitate release of the pharmaceutical agents over certain time periods and/or intervals and can include compositions that enhance delivery of the pharmaceuticals, such as nanoparticle, microsphere or liposome formulations, a variety of which are known in the art and are commercially available.

The dose of the composition comprising a compound of the present disclosure and a pharmaceutical agent generally depends upon the needs of the individual to whom the composition of the disclosure is to be administered. These factors include, for example, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art and given the benefit of the present disclosure.

The compounds of the present disclosure can be therapeutically administered as the neat chemical, but it is preferred to administer a compound of the present disclosure as a pharmaceutical composition or formulation. Thus, the present disclosure provides a pharmaceutical composition comprising a compound of the present disclosure together with a pharmaceutically acceptable diluent or carrier therefor. Also provided is a process of preparing a pharmaceutical composition comprising admixing a compound of the present disclosure with a pharmaceutically acceptable diluent or carrier therefor.

When a compound of the present disclosure is administered as a pharmaceutical to humans and animals, it can be given per se or as a pharmaceutical composition containing, for example, 0.1 to 99.5% (more preferably, 0.5 to 90%) of active ingredient in combination with a pharmaceutically acceptable carrier.

In certain embodiments, the methods of the disclosure include administering to an individual a therapeutically effective amount of a compound of the present disclosure in combination with another pharmaceutically active ingredient. Pharmaceutically active ingredients that may be used can be found in Harrison's Principles of Internal Medicine, 21^{st} Edition, Eds. Fauci et al. McGraw-Hill N.Y., NY; and the Physicians' Desk Reference 70th Edition 2015, Oradell, New Jersey, Medical Economics Co.

Methods delineated herein include those wherein the individual is identified as in need of a particular stated treatment. Identifying an individual in need of such treatment can be in the judgment of an individual or a health care professional and can be subjective (e.g., opinion) or objective (e.g., measurable by a test or diagnostic method). In other methods, the individual is prescreened or identified as in need of such treatment by assessment for a relevant marker or indicator of suitability for such treatment.

The compounds and compositions disclosed in the present disclosure can also be used for prophylaxis in an individual who is at risk developing colorectal cancer. The identification of those patients in need of prophylactic treatment for a colorectal cancer can readily identify such candidate patients, using, for example, clinical tests, physical examination and medical/family history. The individual may have colorectal cancer, may be at risk of developing colorectal cancer, or may have one or more conditions that that cause a subject to have an increased likelihood of developing colorectal cancer.

In an aspect, the present disclosure provides compounds. A compound of the present disclosure may have the following structure:

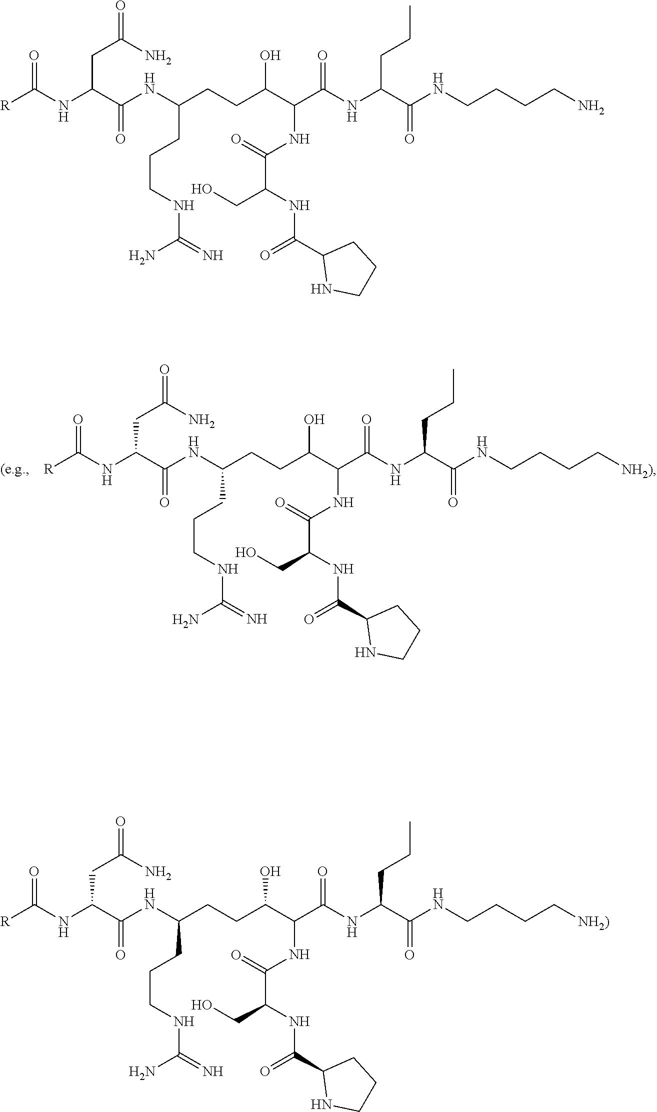

or a salt thereof, wherein R is a substituted or unsubstituted aliphatic group, with the proviso the compound does not have the following structure:

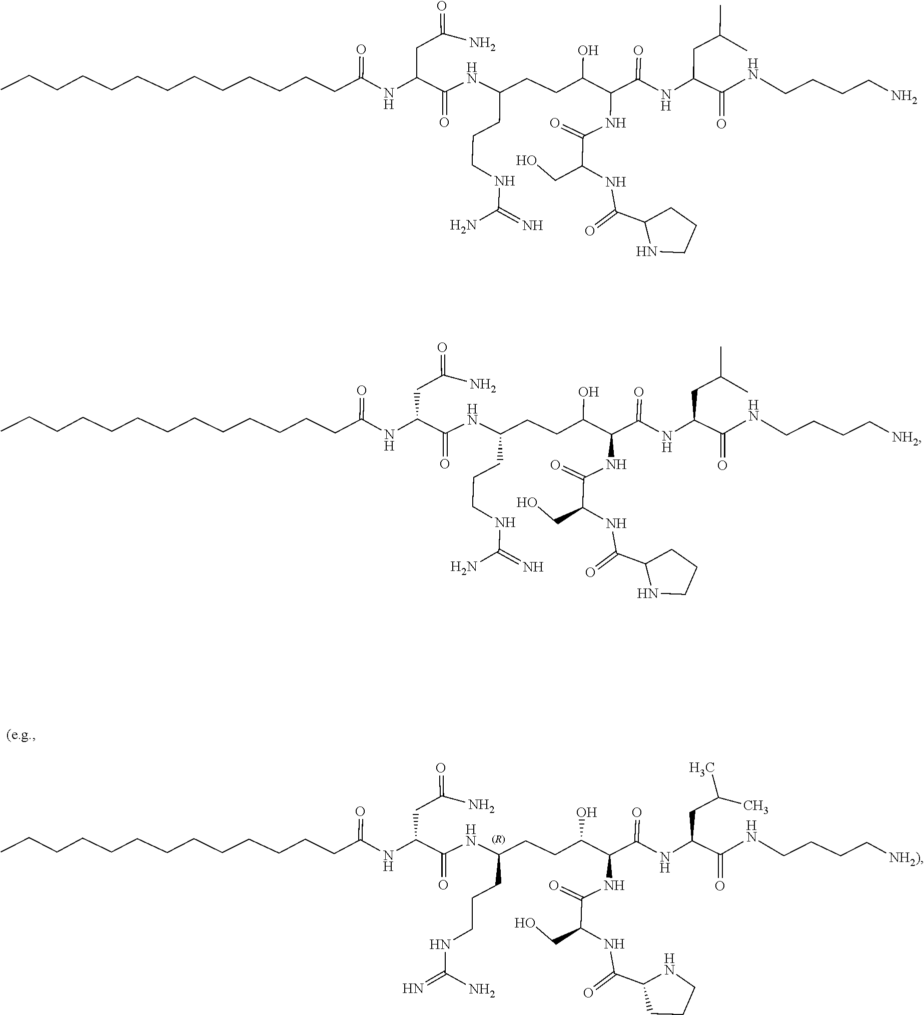

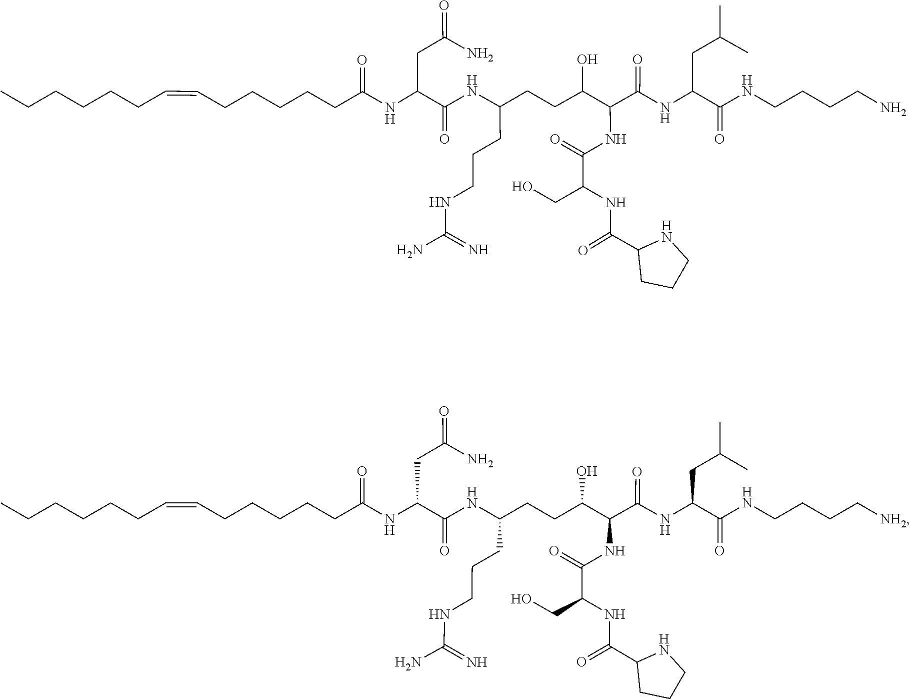 [SMILES: CCCCCC/C=C\CCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CCCCCC/C=C\CCCCCC(=O)N[C@H](CC(N)=O)C(=O)N[C@@H](CCCNC(=N)N)CC[C@H](O)[C@H](NC(=O)[C@H](CO)NC(=O)C1CCCN1)C(=O)N[C@@H](CC(C)C)C(=O)NCCCCN]

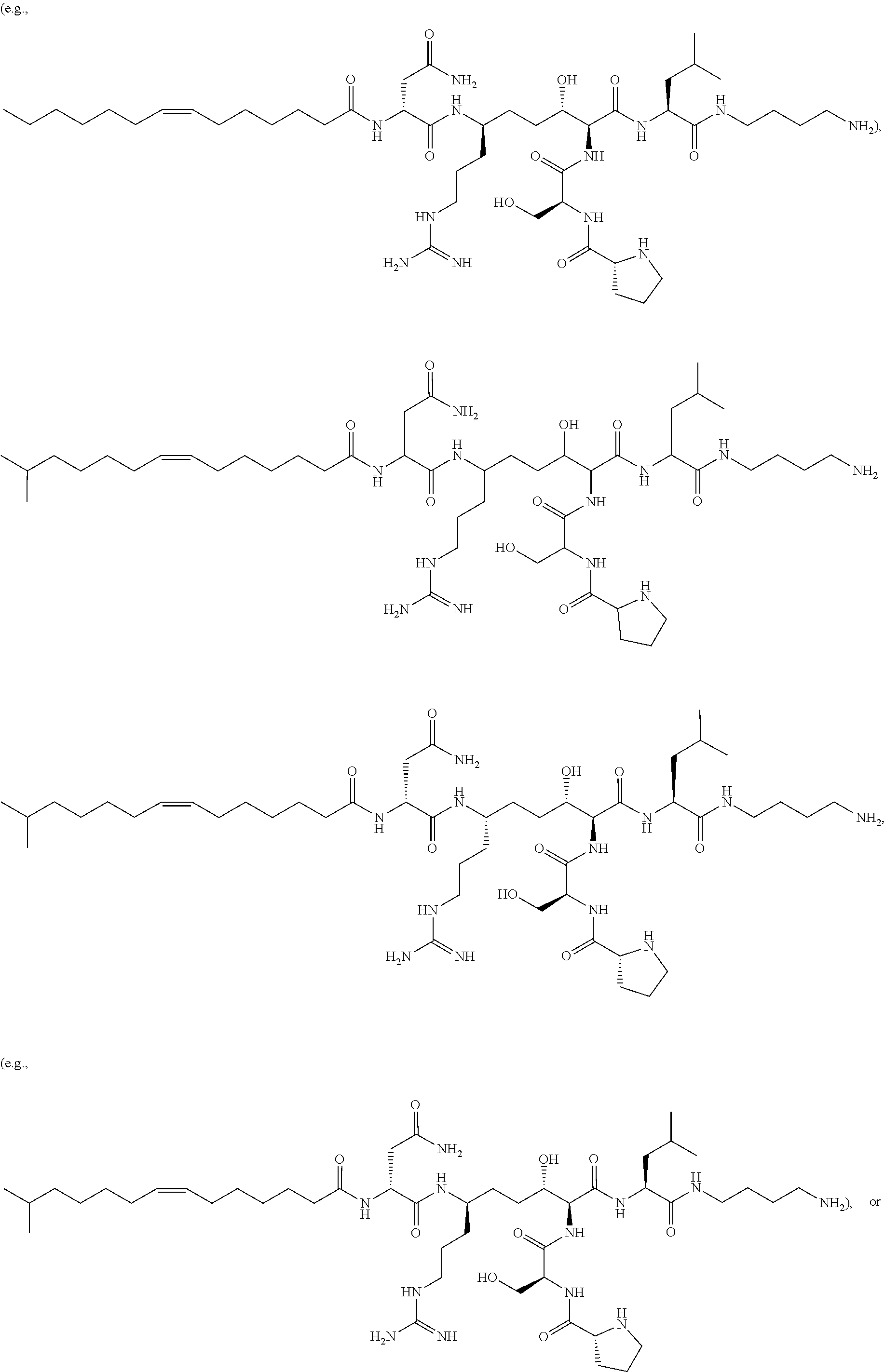

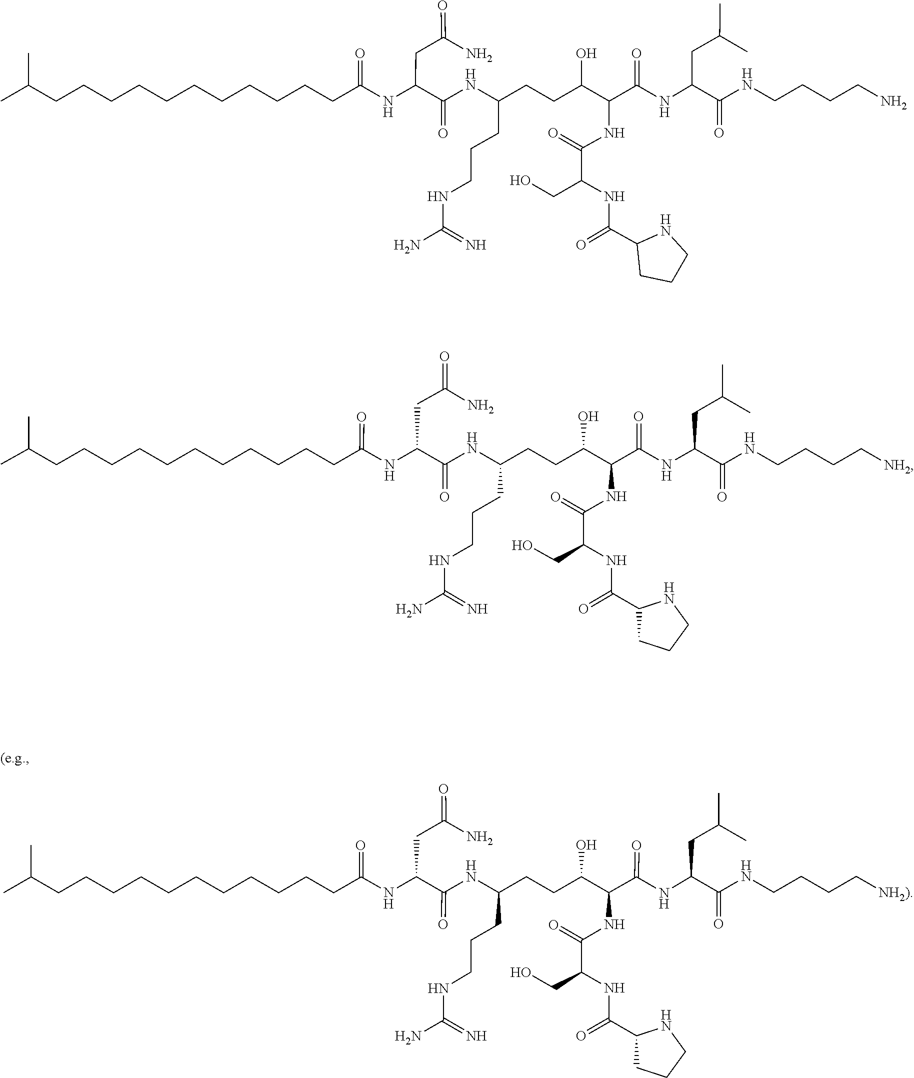

Various R groups may be used. For example, the R group may be an aliphatic group 1 to 25 carbons atoms in length (e.g., 1 to 22 carbon atoms in length, 1 to 18 carbon atoms in length, or 7 to 25 carbon atoms in length) (e.g., 1, 2, 3, 4, 5, 6, 7, 8, 9, 10, 11, 12, 13, 14, 15, 16, 17, 18, 19, 20, 21, 22, 23, 24, or 25 carbon atoms in length). The aliphatic group may be substituted or unsubstituted. The aliphatic group may have one or more aromatic rings (e.g., aryl or heteroaryl groups), one or more pi bonds (e.g., one or more double bonds and/or one or more triple bonds), one or more hydroxy groups, and/or one or more halogens (e.g., fluoro groups). The one or more pi bonds may be conjugated. For example, aromatic rings may be pendent substituents, terminal substituents, or the aromatic rings may interrupt a linear aliphatic chain such that a portion of the aliphatic group is one substituent on the aromatic ring and the remaining portion of the aliphatic chain is a different substituent on the aromatic ring

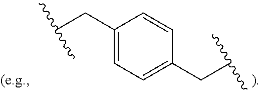 [SMILES: *Cc1ccc(C*)cc1]

The aromatic rings may have one or more additional substituents. For example, R may have the following structure:

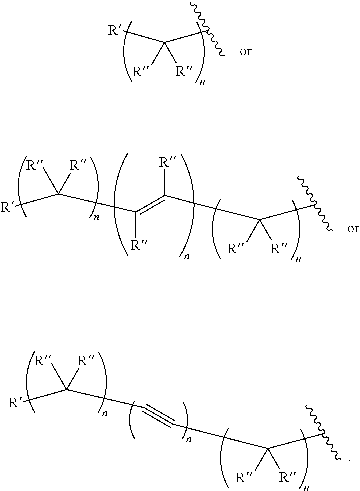

R′ is hydrogen, a halogen (e.g., bromo, fluoro, or the like), an aryl group (e.g., phenyl, napthyl, or the like), a methyl group, halogenated methyl group (e.g., —CF₃), an amino group, or a hydroxy, or the like. An R′ group may be unsubstituted or further substituted. R″ is hydrogen, a halogen (e.g., bromo, fluoro, or the like), an aryl group (e.g., phenyl, napthyl, or the like), a methyl group, halogenated methyl group (e.g., —CF₃), a hydroxy, or the like. Each R″ may be the same or different. As an illustrative example, on a carbon atom substituted with two R″ groups, one R″ is hydrogen, and one R″ is methyl. In another illustrative example, if R is

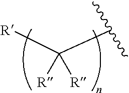

and n is 2, each of the four resulting R″ groups may be the same or different. For example, one R″ is a methyl, one R″ is hydrogen, one R″ is fluoro, and the final R″ is bromo. Other combinations of substituents on these genuses are contemplated by this disclosure. Each n is independently 0 to 18 (e.g., 0, 1, 2, 3, 4, 5, 6, 7, 8, 9, 10, 11, 12, 13, 14, 15, 16, 17, 18). In various examples, R may have the following structure:

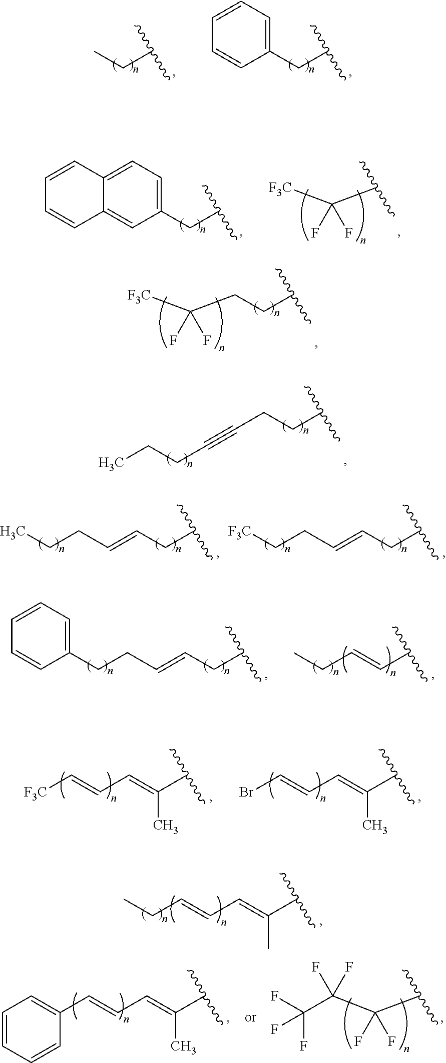

wherein each n is independently 0 to 18 (e.g., 0, 1, 2, 3, 4, 5, 6, 7, 8, 9, 10, 11, 12, 13, 14, 15, 16, 17, 18). Additional examples of R groups include, but are not limited to:

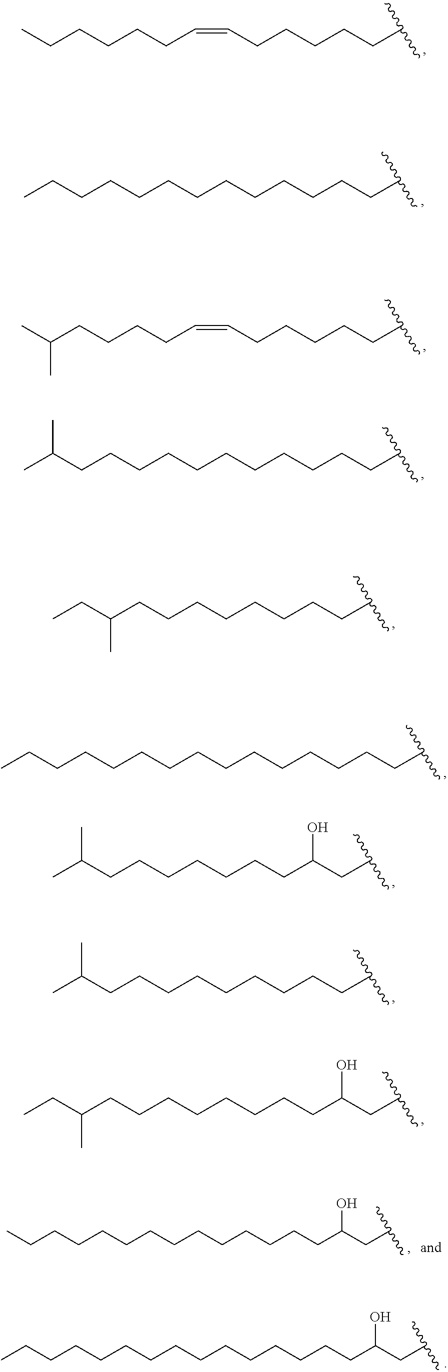

For example, a compound of the present disclosure may have the following structure:

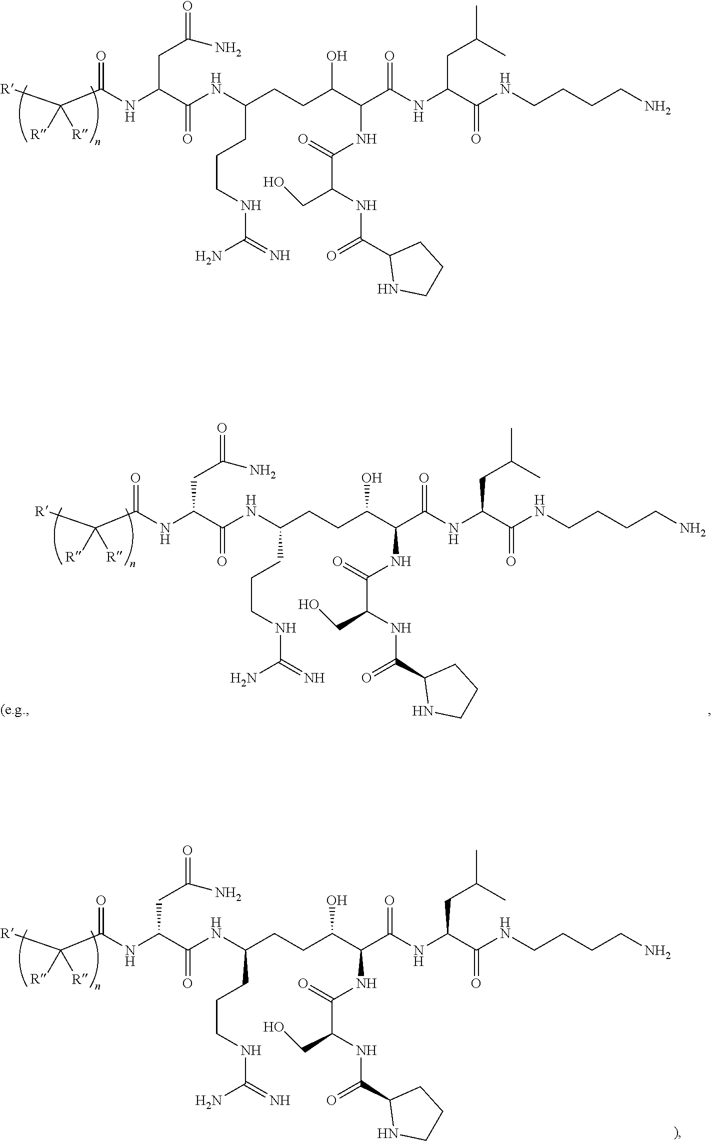

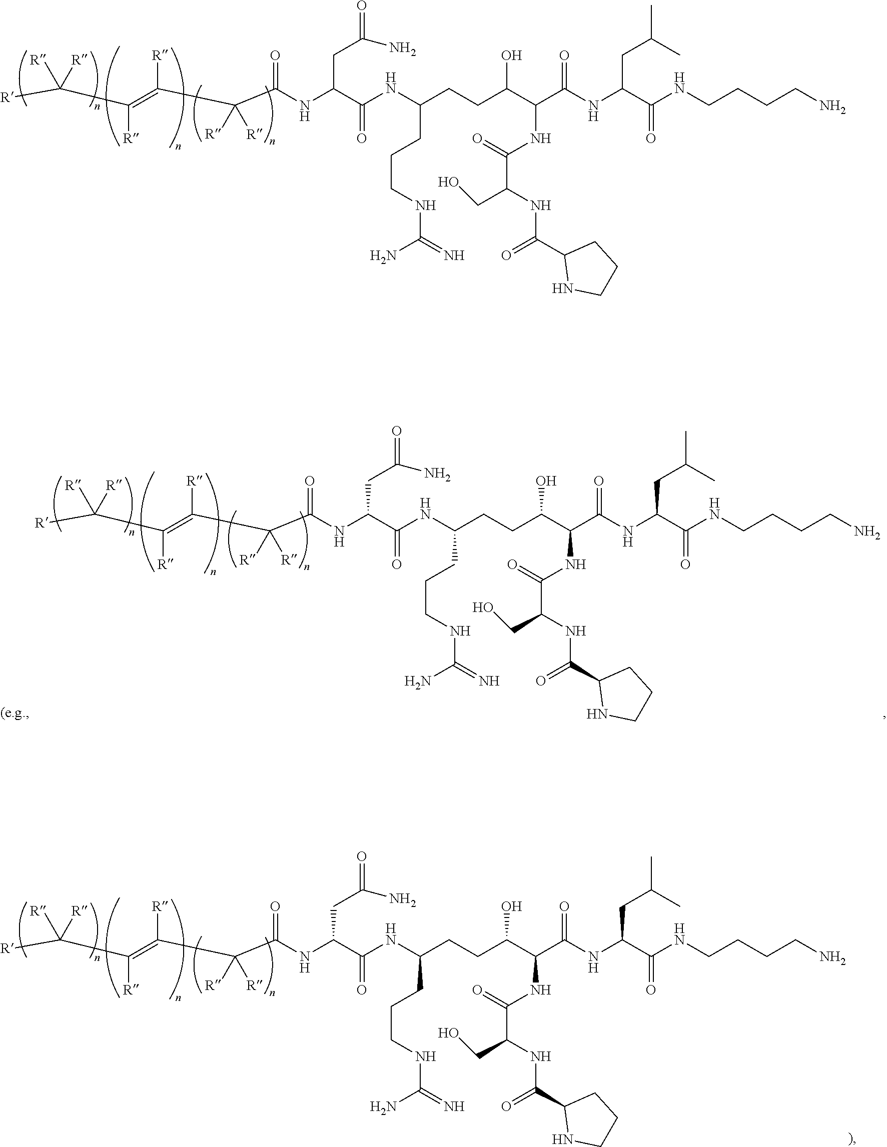

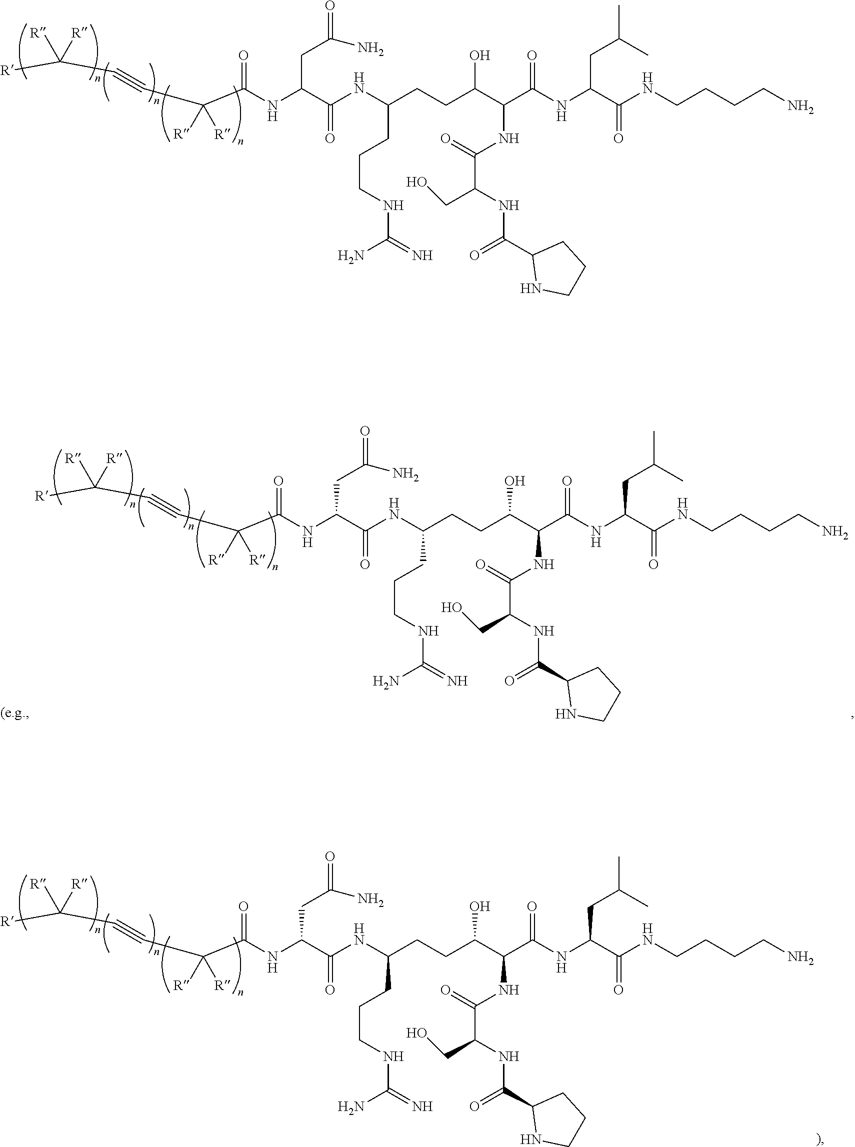

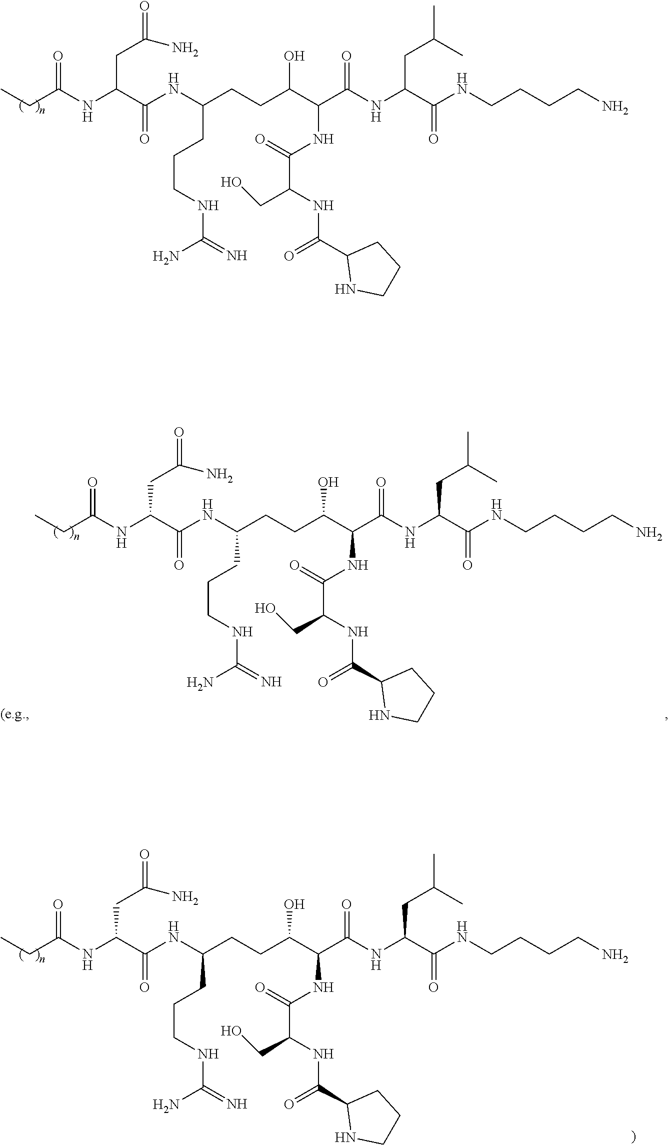

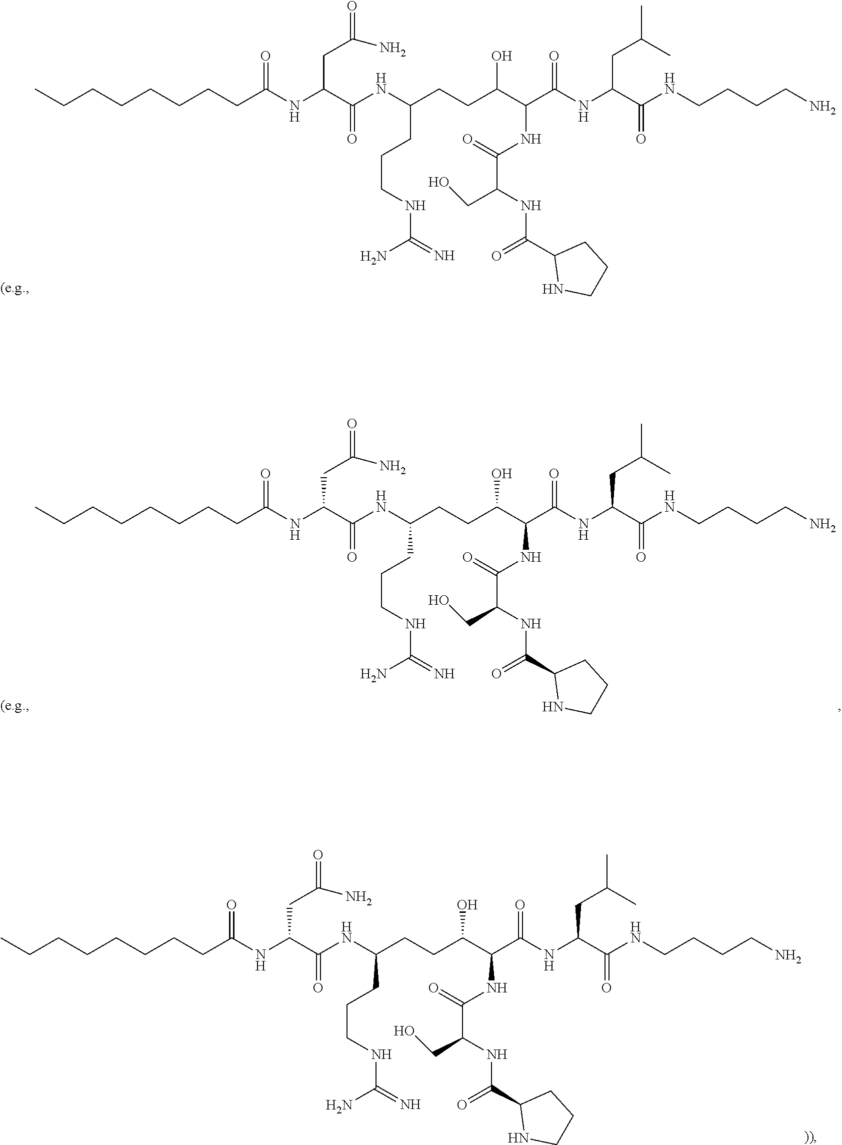 [SMILES: CCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]

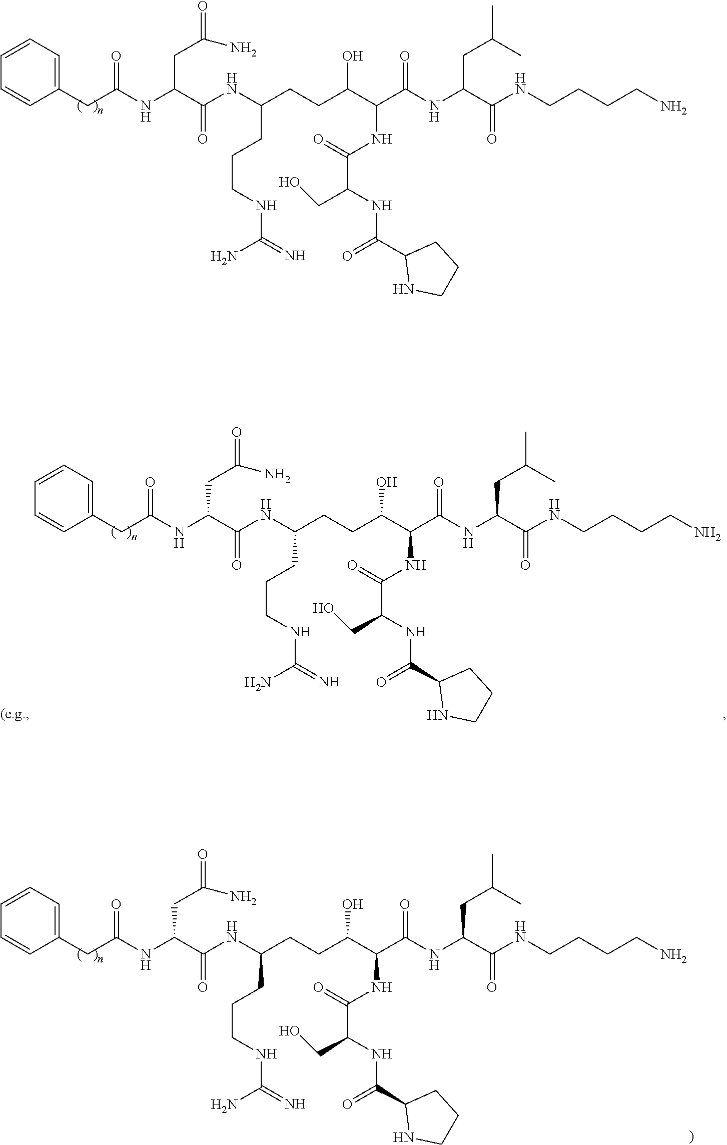

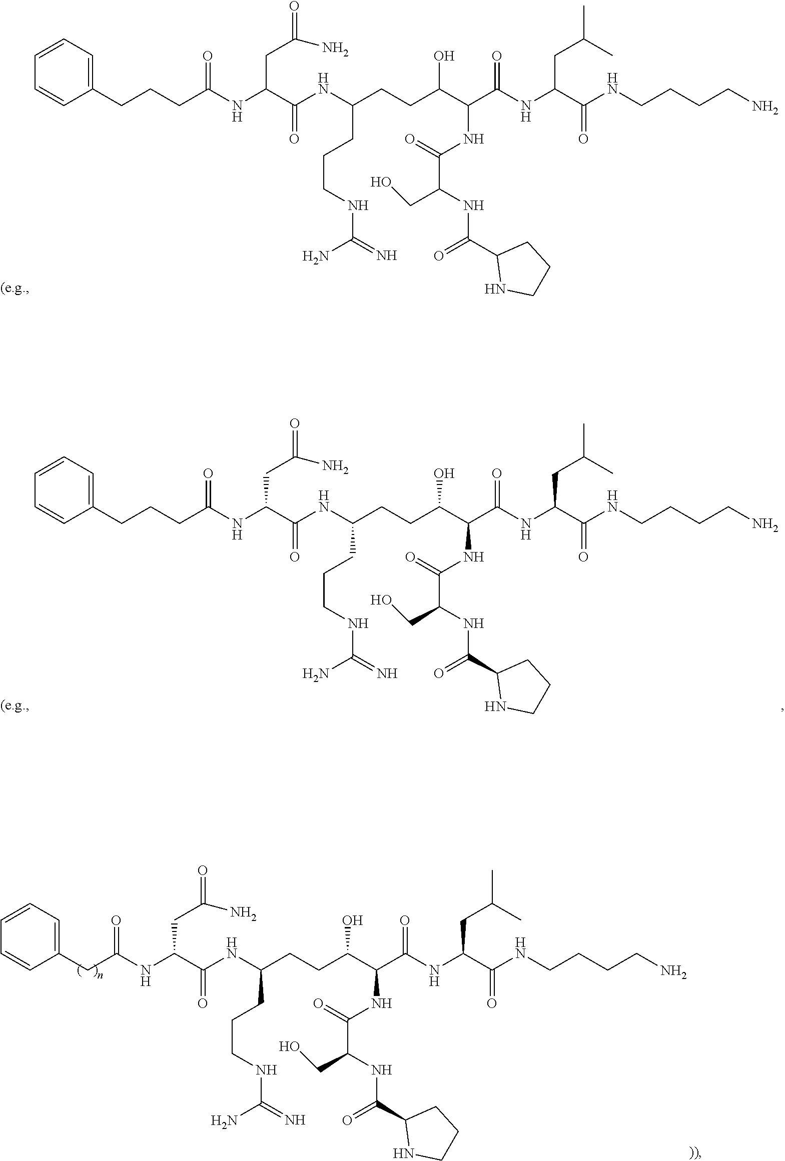

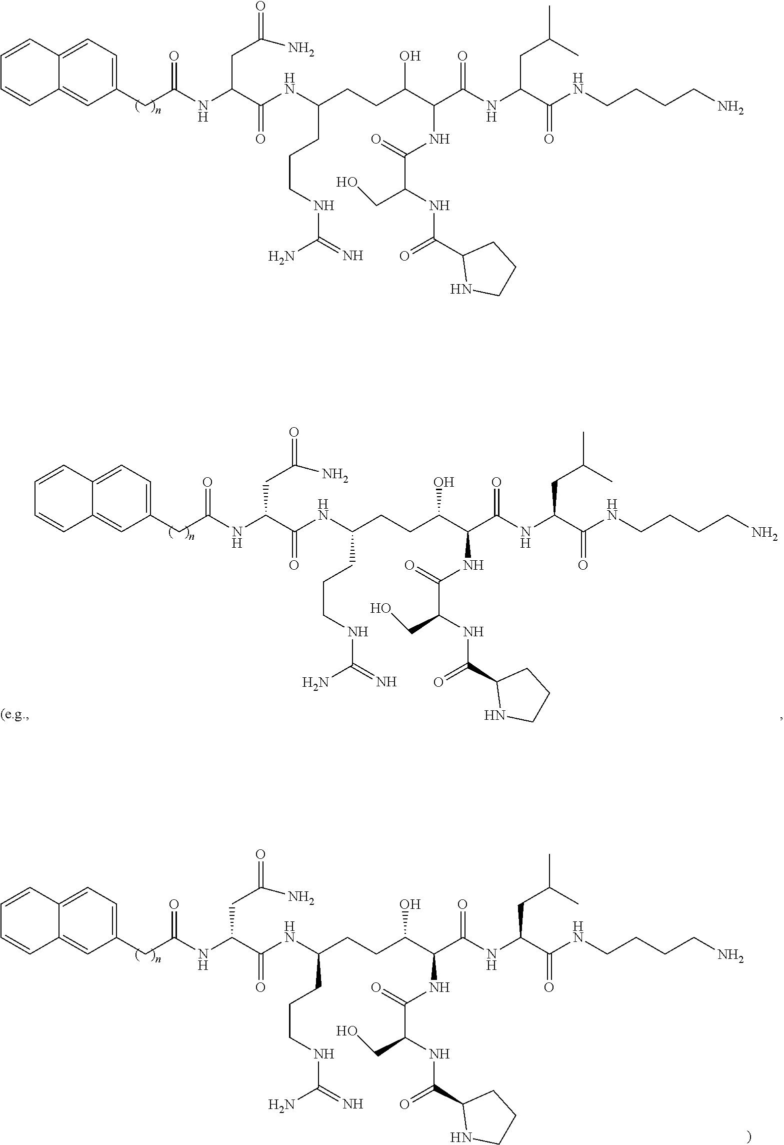

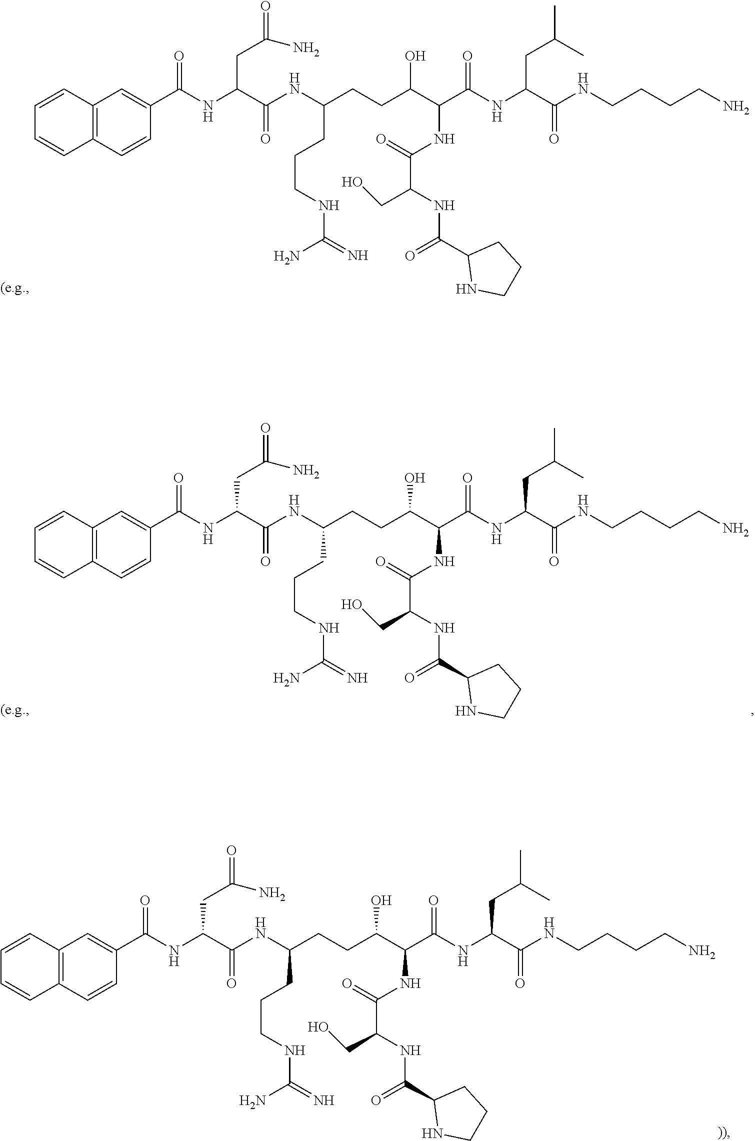 [SMILES: CC(C)CC(NC(=O)C(NC(=O)C(CO)NC(=O)C1CCCN1)C(O)CCC(CCCNC(=N)N)NC(=O)C(CC(N)=O)NC(=O)c1ccc2ccccc2c1)C(=O)NCCCCN ; CC(C)C[C@H](NC(=O)[C@@H](NC(=O)[C@H](CO)NC(=O)[C@H]1CCCN1)[C@@H](O)CC[C@H](CCCNC(=N)N)NC(=O)[C@@H](CC(N)=O)NC(=O)c1ccc2ccccc2c1)C(=O)NCCCCN ; CC(C)C[C@H](NC(=O)[C@@H](NC(=O)[C@H](CO)NC(=O)[C@H]1CCCN1)[C@@H](O)CC[C@@H](CCCNC(=N)N)NC(=O)[C@@H](CC(N)=O)NC(=O)c1ccc2ccccc2c1)C(=O)NCCCCN]

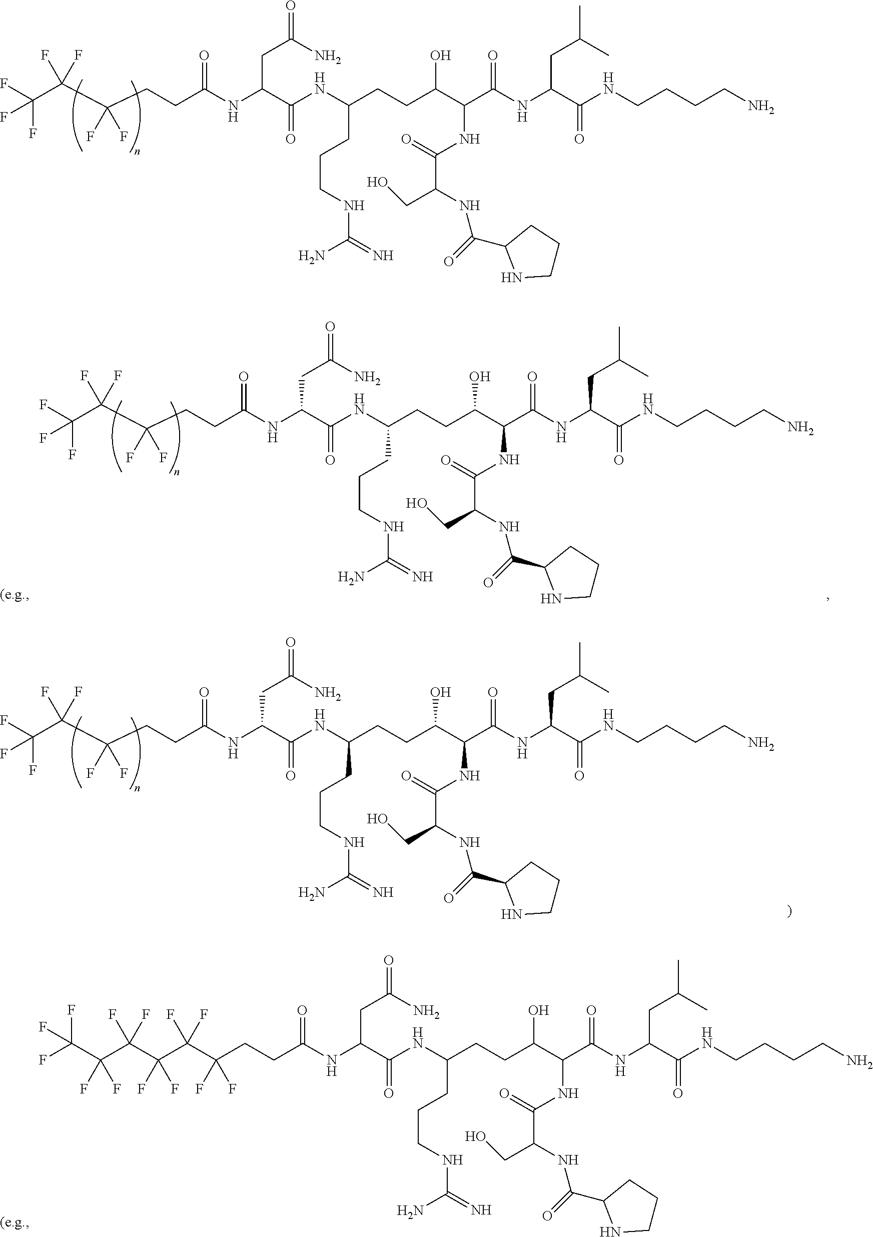

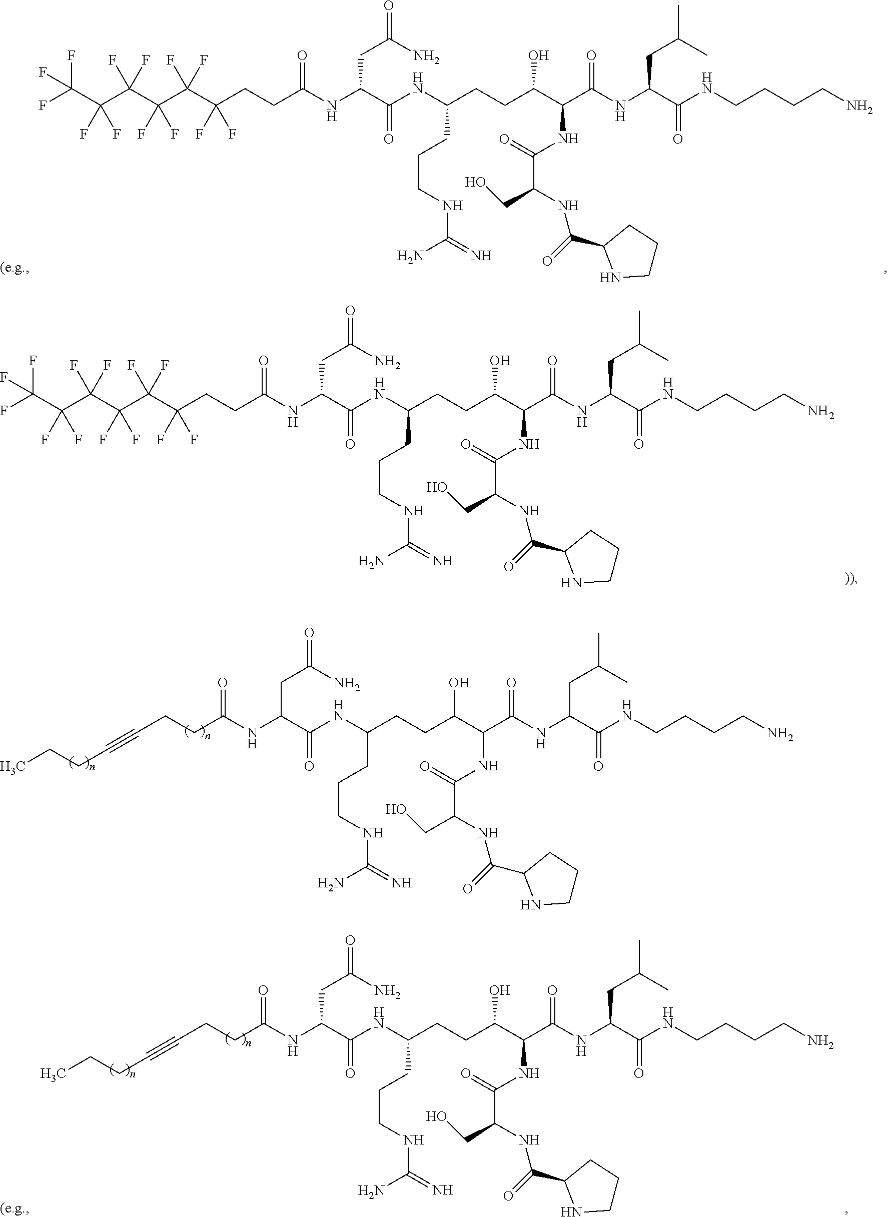

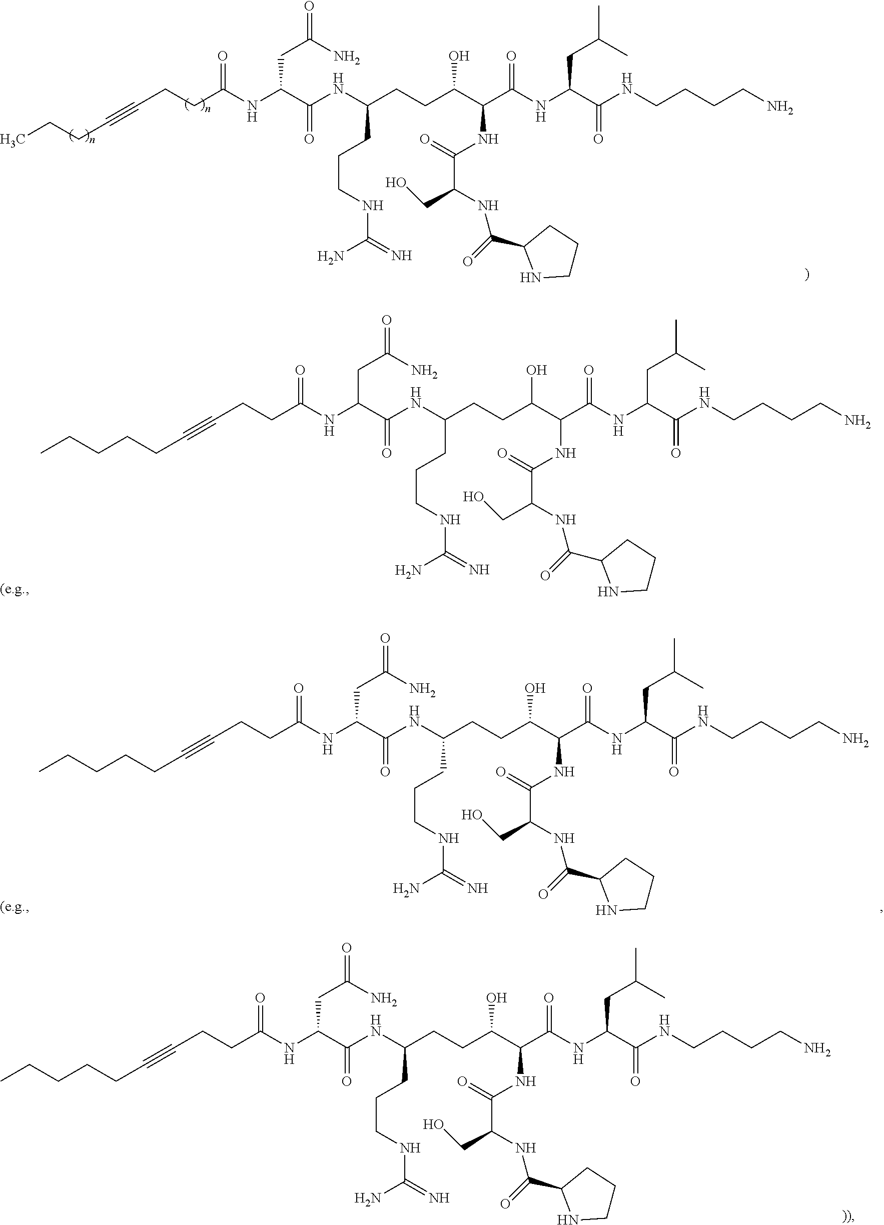

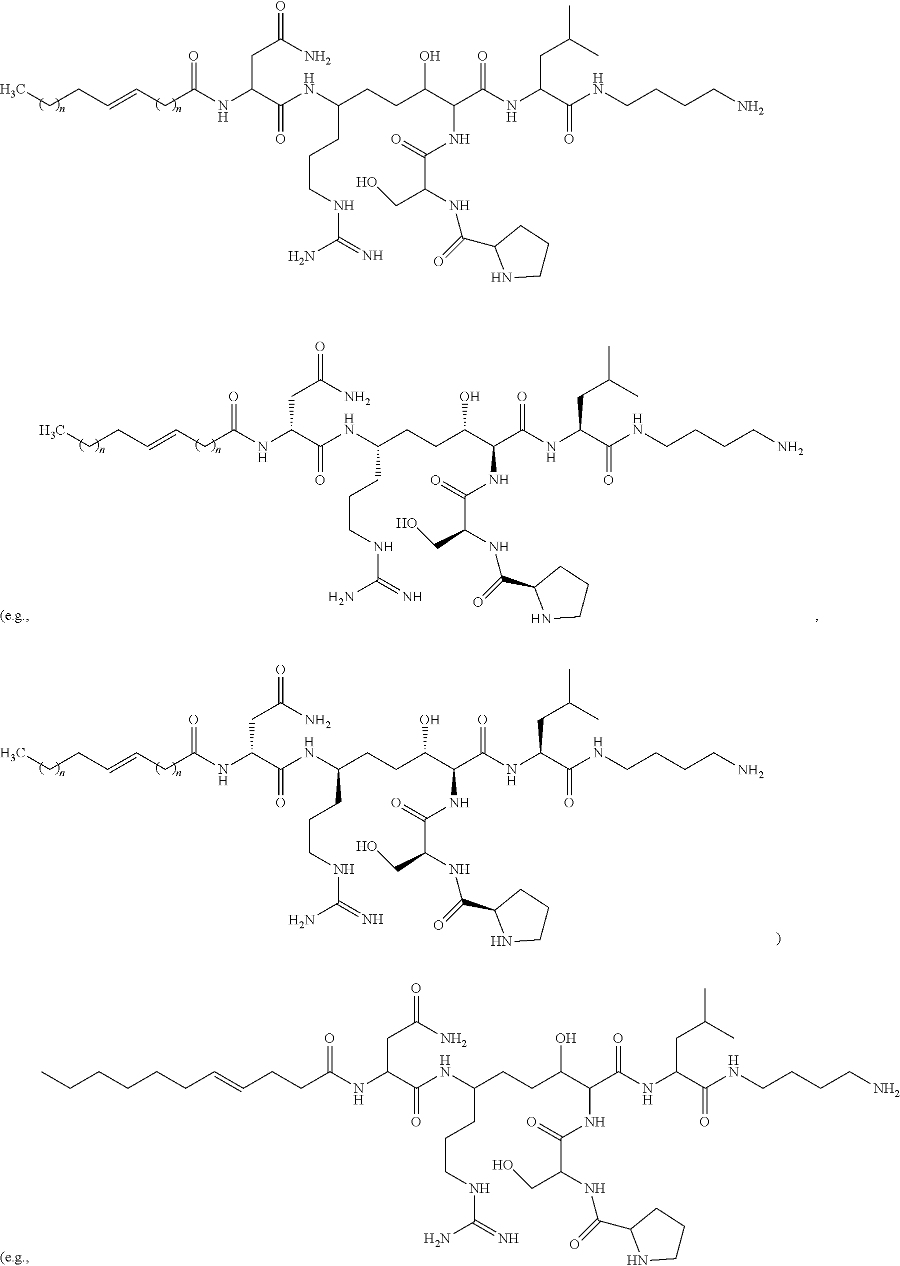

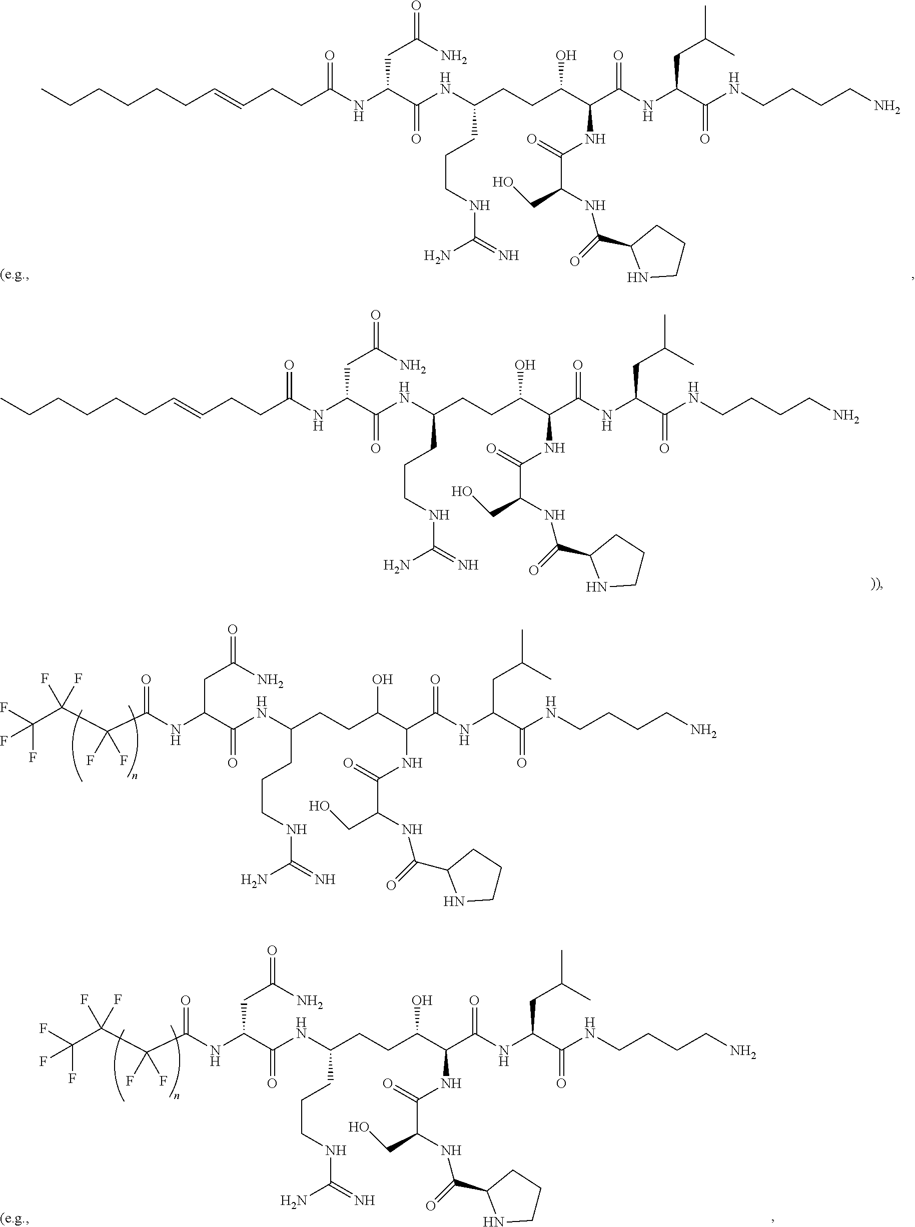

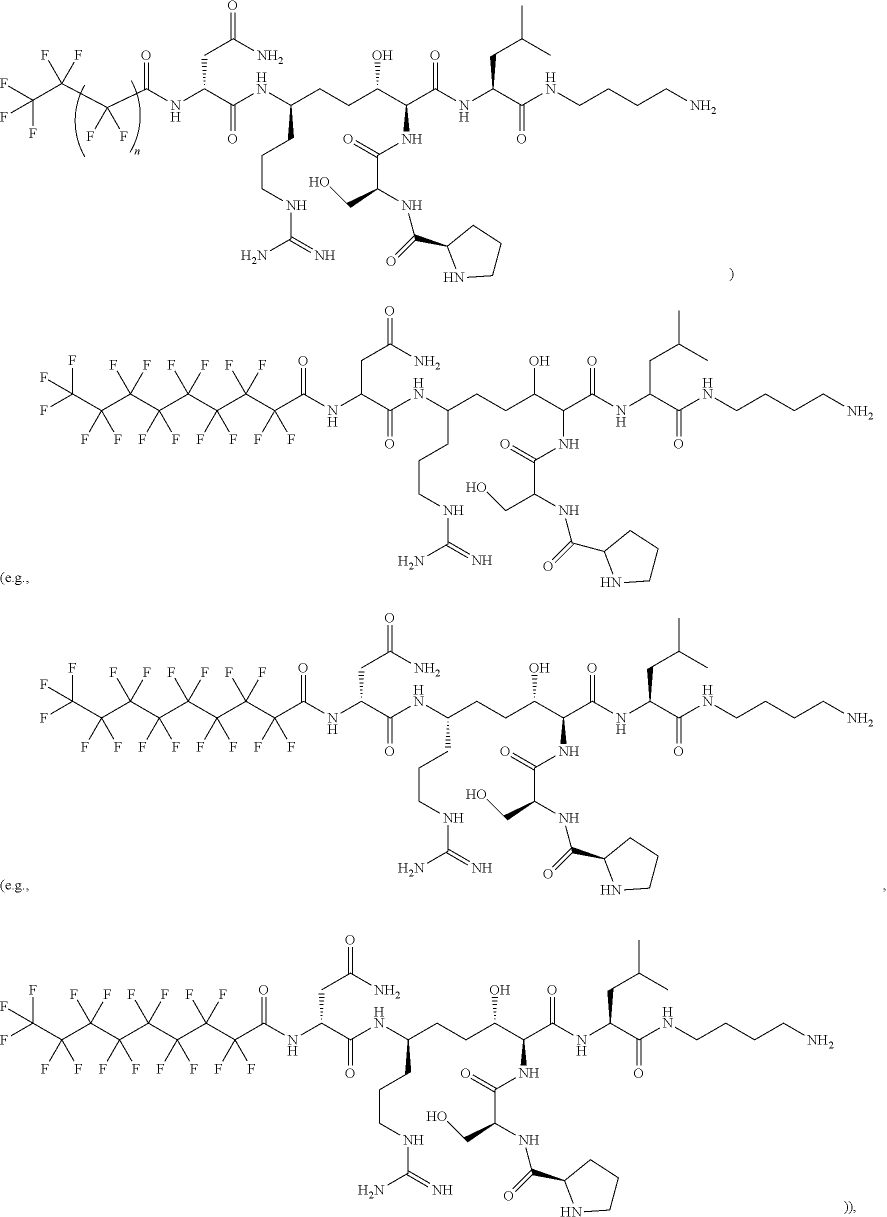

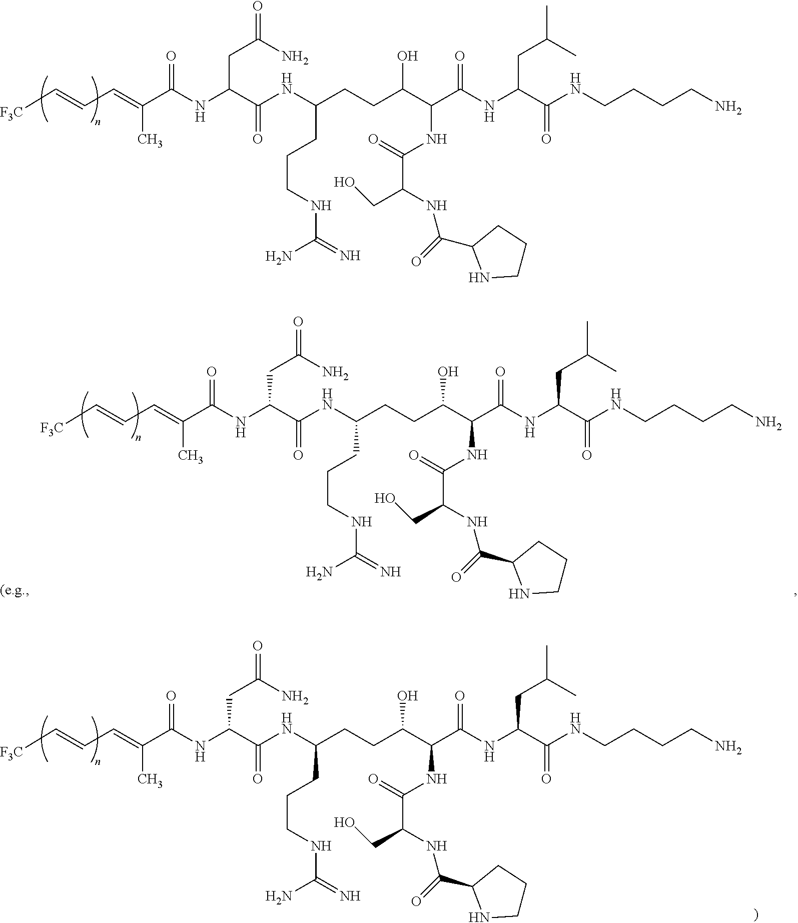

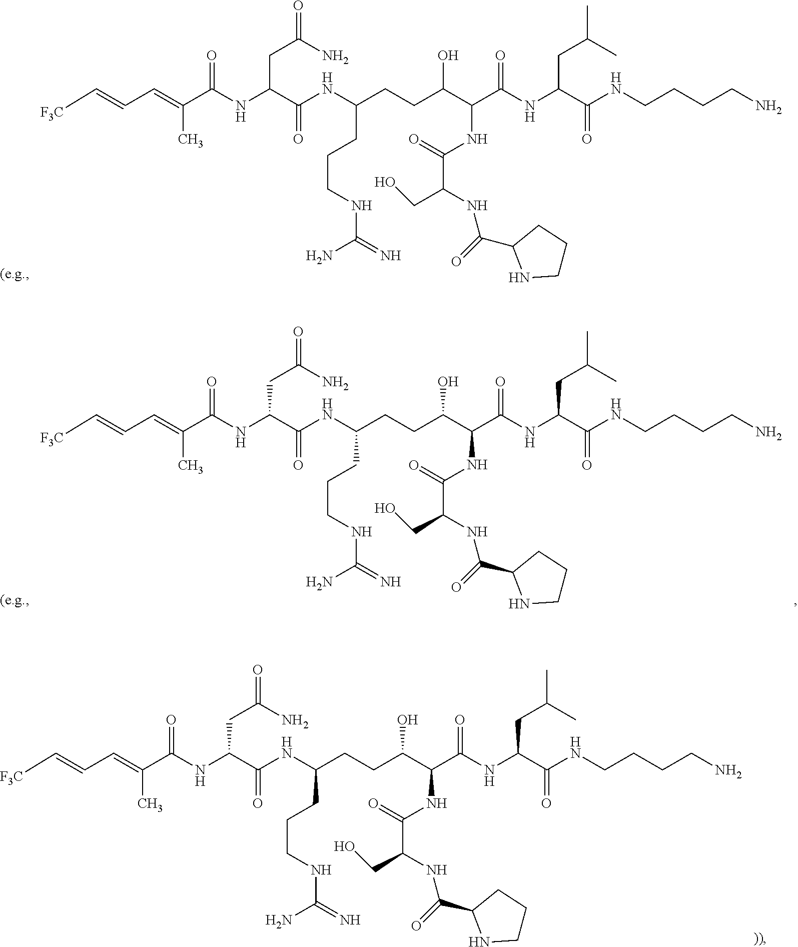 [SMILES: C/C(=C\C=C\C(F)(F)F)C(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; C/C(=C\C=C\C(F)(F)F)C(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; C/C(=C\C=C\C(F)(F)F)C(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]

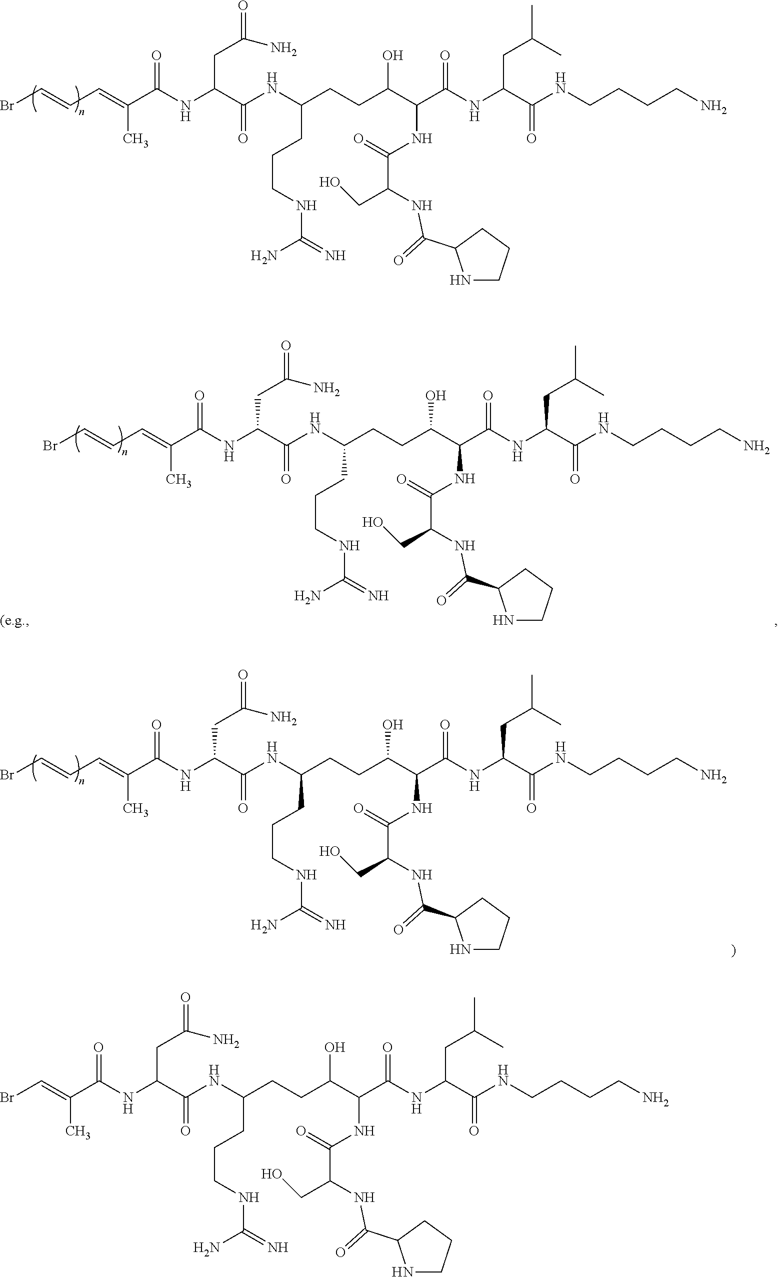

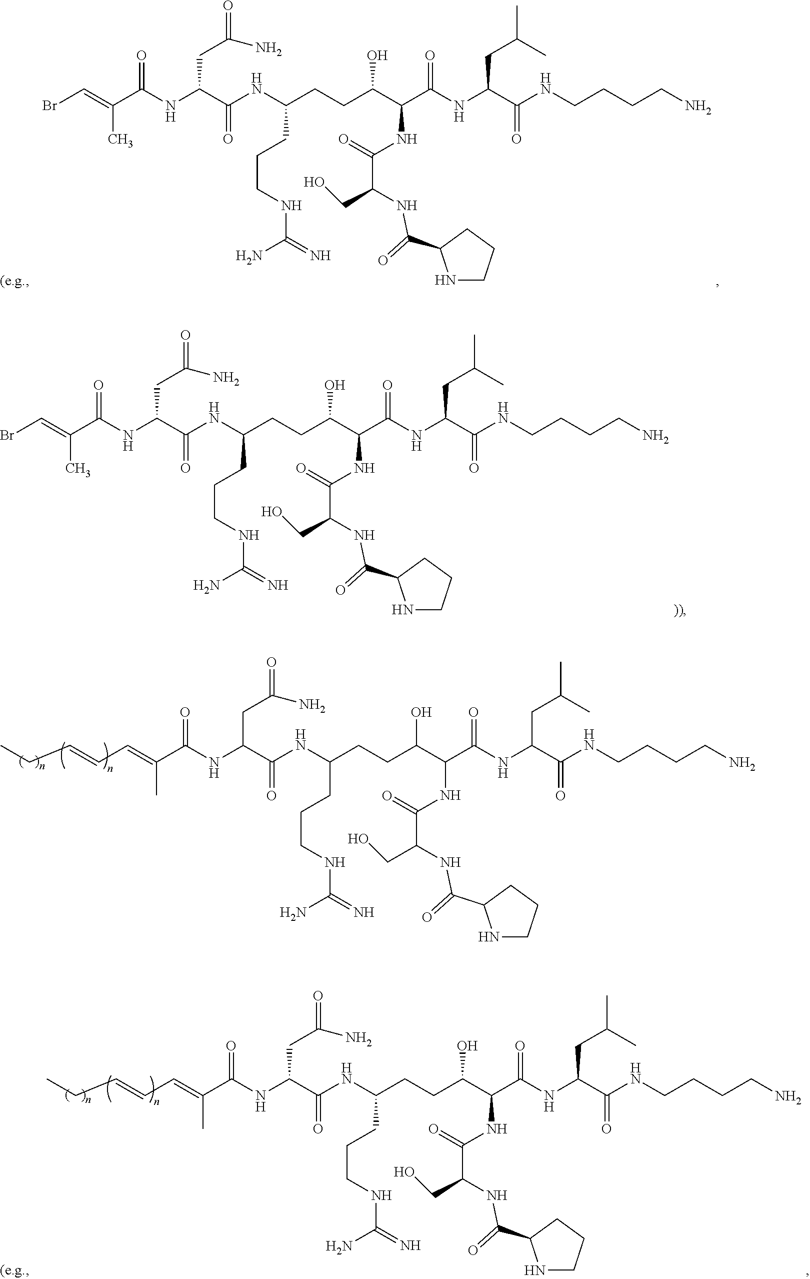

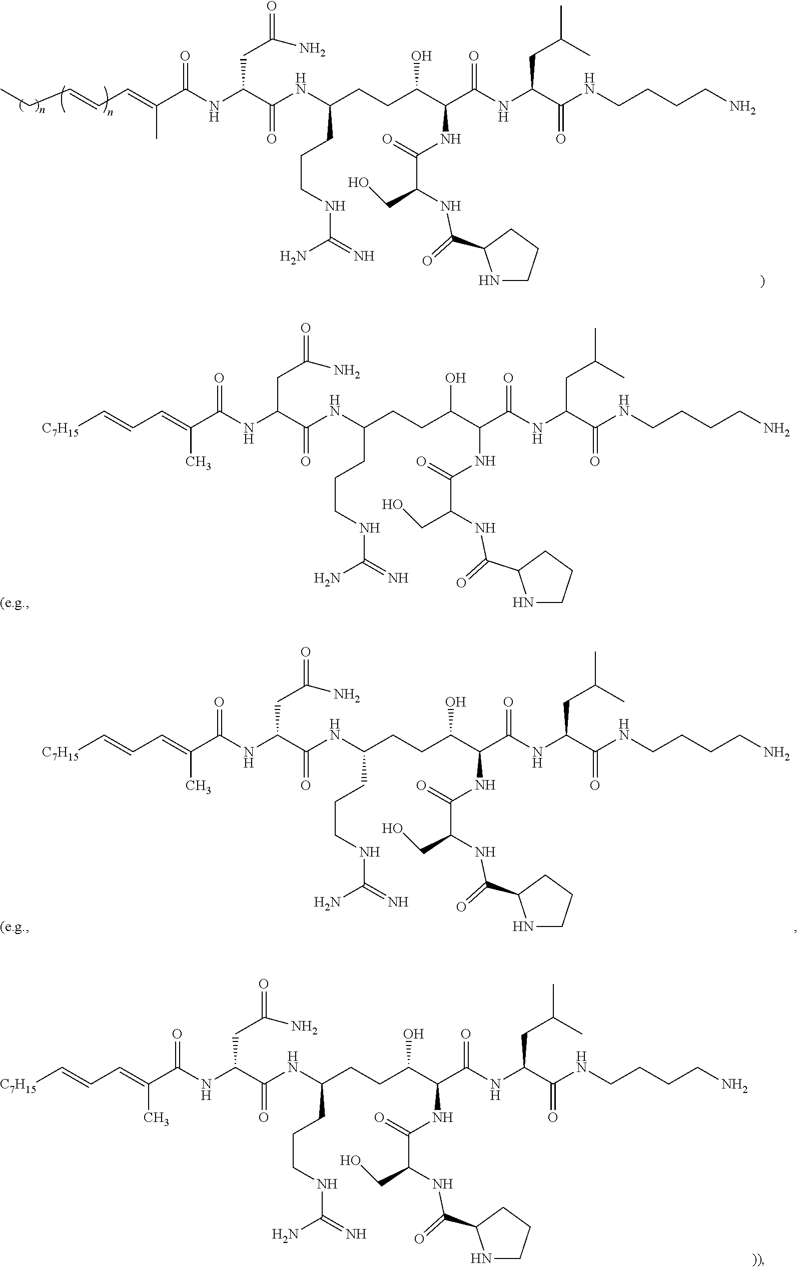

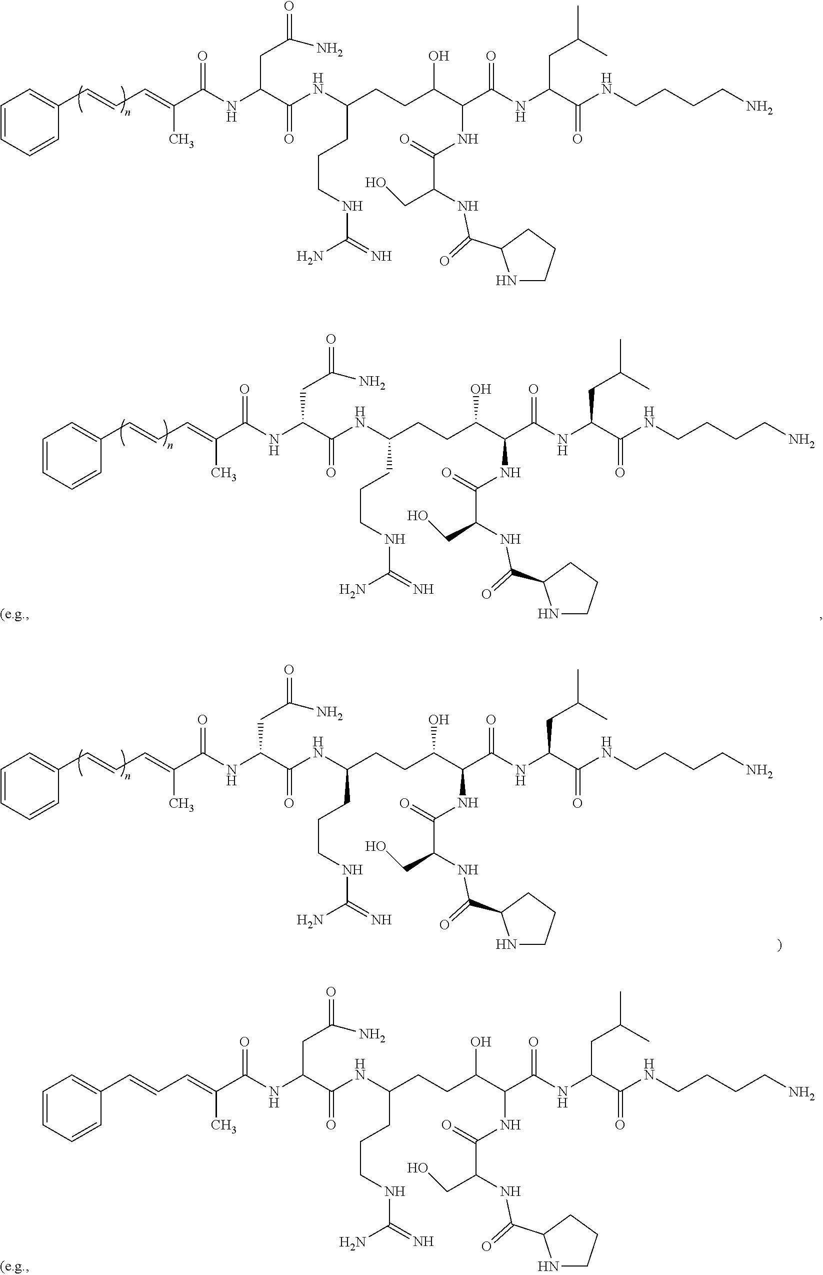

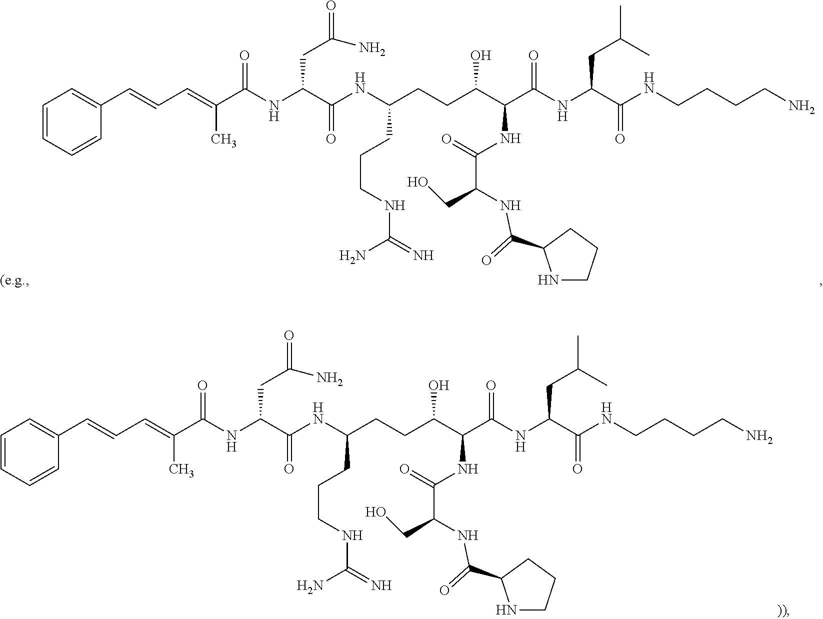 [SMILES: C/C(=C\C=C\c1ccccc1)C(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; C/C(=C\C=C\c1ccccc1)C(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]

or any combination thereof, wherein n is 0 to 18.

In various examples, the compound (e.g., the prodrug antibiotic) has the following structure:

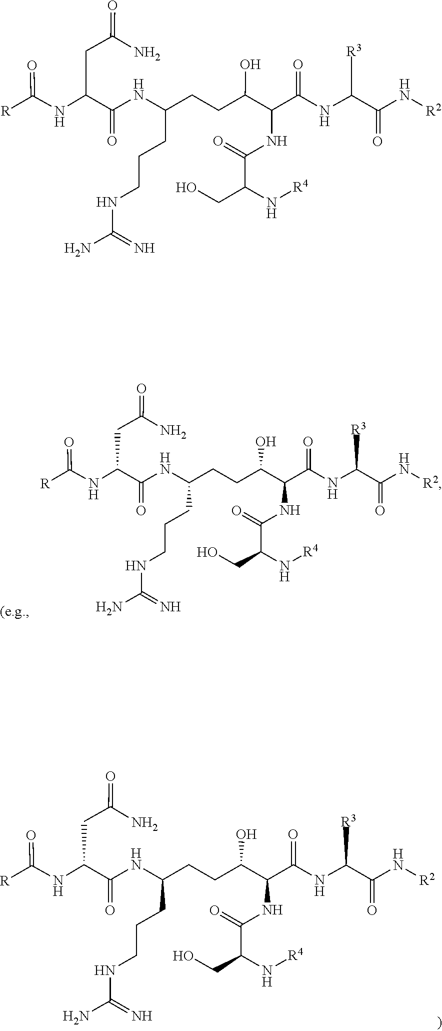

where is R may be an aliphatic group 1 to 25 carbons atoms in length (e.g., 1 to 22 carbon atoms in length, 1 to 18 carbon atoms in length, or 7 to 25 carbon atoms in length) (e.g., 1, 2, 3, 4, 5, 6, 7, 8, 9, 10, 11, 12, 13, 14, 15, 16, 17, 18, 19, 20, 21, 22, 23, 24, or 25 carbon atoms in length). The aliphatic group may be substituted or unsubstituted. The aliphatic group may have one or more aromatic rings (e.g., aryl or heteroaryl groups), one or more pi bonds (e.g., one or more double bonds and/or one or more triple bonds), one or more hydroxy groups, and/or one or more halogens (e.g., fluoro groups). The one or more pi bonds may be conjugated. For example, aromatic rings may be pendent substituents, terminal substituents, or the aromatic rings may interrupt a linear aliphatic chain such that a portion of the aliphatic group is one substituent on the aromatic ring and the remaining portion of the aliphatic chain is a different substituent on the aromatic ring

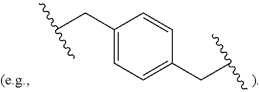 [SMILES: *Cc1ccc(C*)cc1]

The aromatic rings may have one or more additional substituents. For example, R may have the following structure:

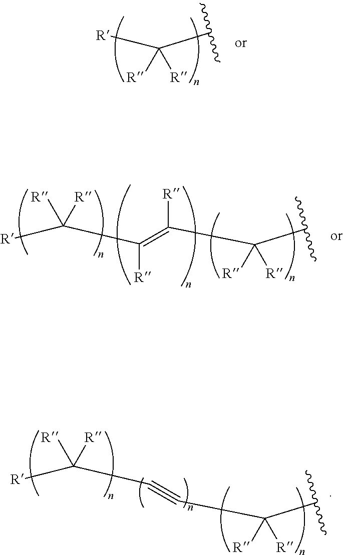

R′ is hydrogen, a halogen (e.g., bromo, fluoro, or the like), an aryl group (e.g., phenyl, napthyl, or the like), a methyl group, halogenated methyl group (e.g., —CF₃), an amino group, or a hydroxy, or the like. An R′ group may be unsubstituted or further substituted. R″ is hydrogen, a halogen (e.g., bromo, fluoro, or the like), an aryl group (e.g., phenyl, napthyl, or the like), a methyl group, halogenated methyl group (e.g., —CF₃), a hydroxy, or the like. Each R″ may be the same or different. As an illustrative example, on a carbon atom substituted with two R″ groups, one R″ is hydrogen, and one R″ is methyl. In another illustrative example, if R is

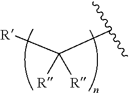

and n is 2, each of the four resulting R″ groups may be the same or different. For example, one R″ is a methyl, one R″ is hydrogen, one R″ is fluoro, and the final R″ is bromo. Other combinations of substituents on these genuses are contemplated by this disclosure. Each n is independently 0 to 18 (e.g., 0, 1, 2, 3, 4, 5, 6, 7, 8, 9, 10, 11, 12, 13, 14, 15, 16, 17, 18). In various examples, R may have the following structure:

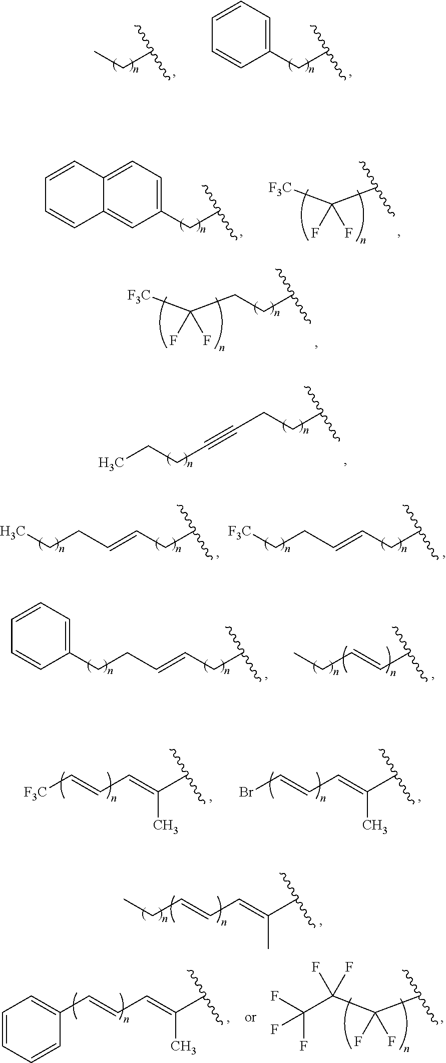

wherein each n is independently 0 to 18 (e.g., 0, 1, 2, 3, 4, 5, 6, 7, 8, 9, 10, 11, 12, 13, 14, 15, 16, 17, 18). Additional examples of R groups include, but are not limited to:

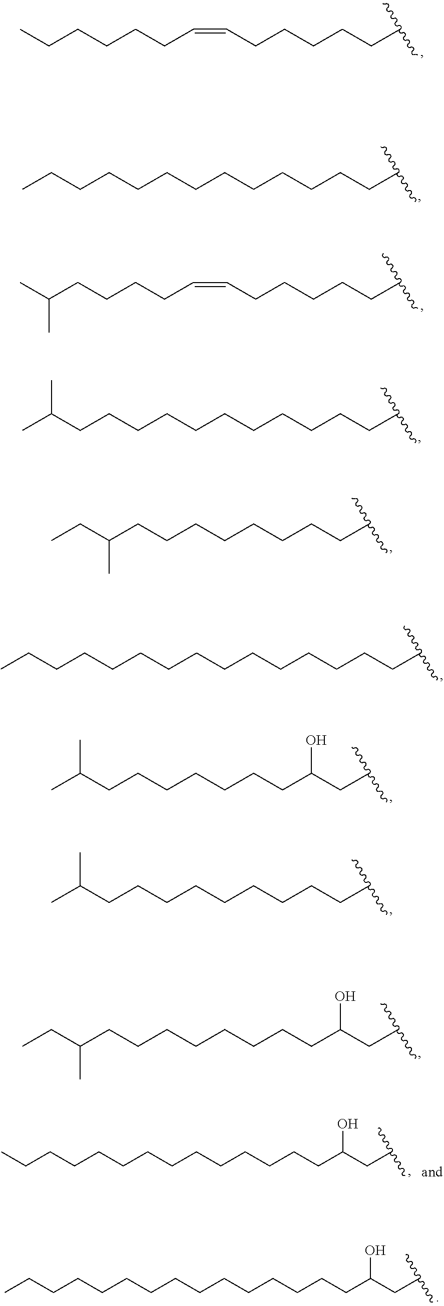

The R² group may be an amino acid residue (e.g., a canonical or noncanonical amino acid residue), a substituted or unsubstituted aliphatic group, or H. Examples of R² groups include, but are not limited to,

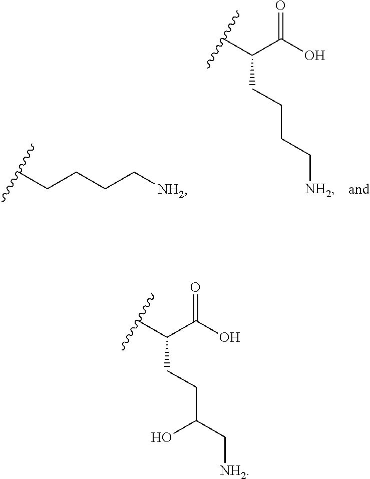 [SMILES: *CCCCN ; *[C@@H](CCCCN)C(=O)O ; *[C@@H](CCC(O)CN)C(=O)O]

The R³ group may be a substituted or unsubstituted aliphatic group. In various other examples, the R³ is the side chain of an amino acid (e.g., a canonical or noncanonical amino acid). For example, R³ may be the side chain of a hydrophobic amino acid. Examples of R³ groups include, but are not limited to,

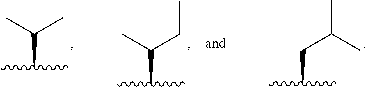

The R⁴ group may be a substituted or unsubstituted acyl group or H. In various examples, the R⁴ group is an amino acid residue (e.g., a canonical or noncanonical amino acid residue). For example, R⁴ may be a proline residue

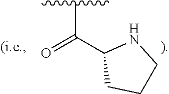 [SMILES: *C(=O)[C@H]1CCCN1]

Examples of compounds (e.g., the prodrug antibiotic) include, but are not limited to,

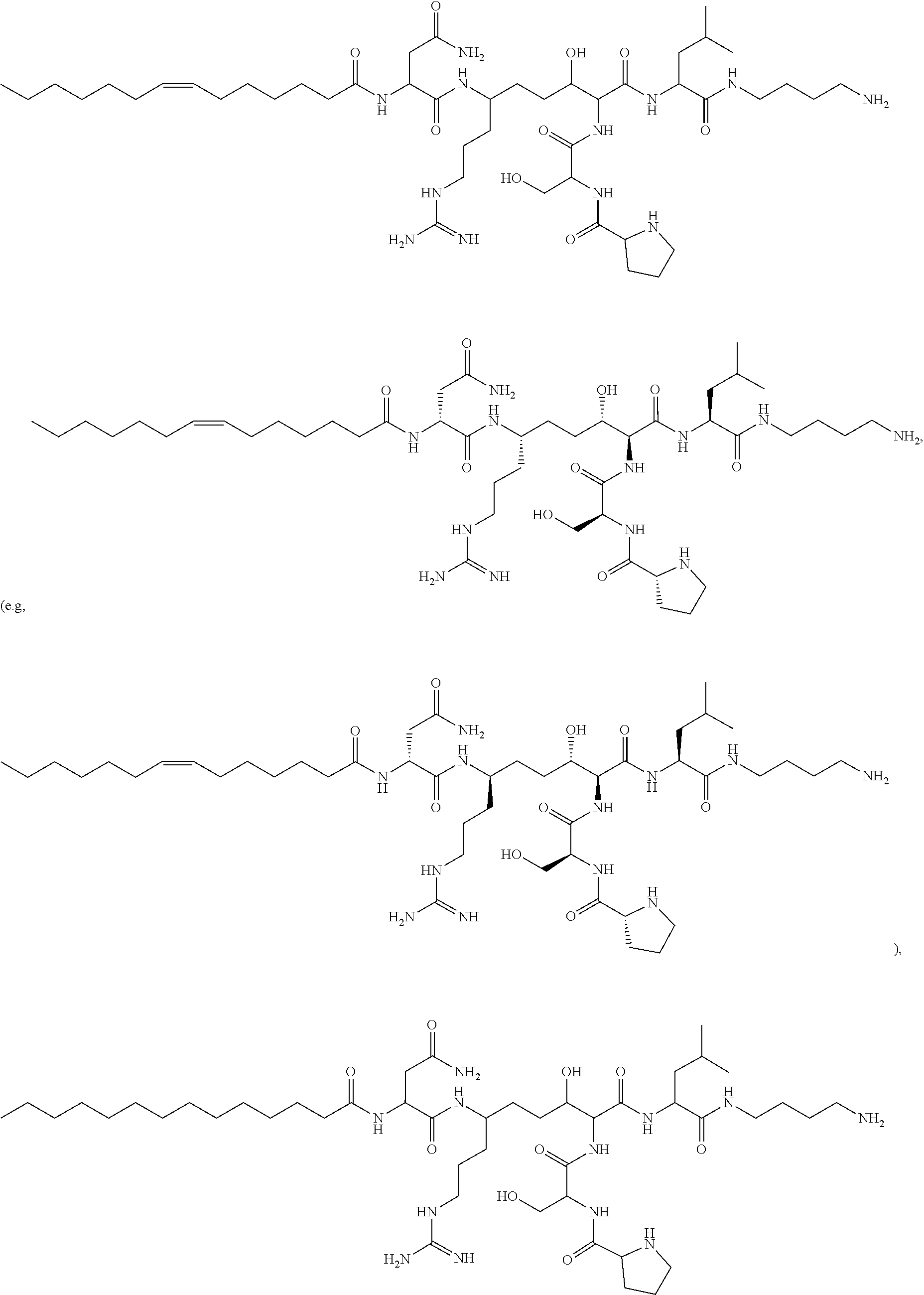

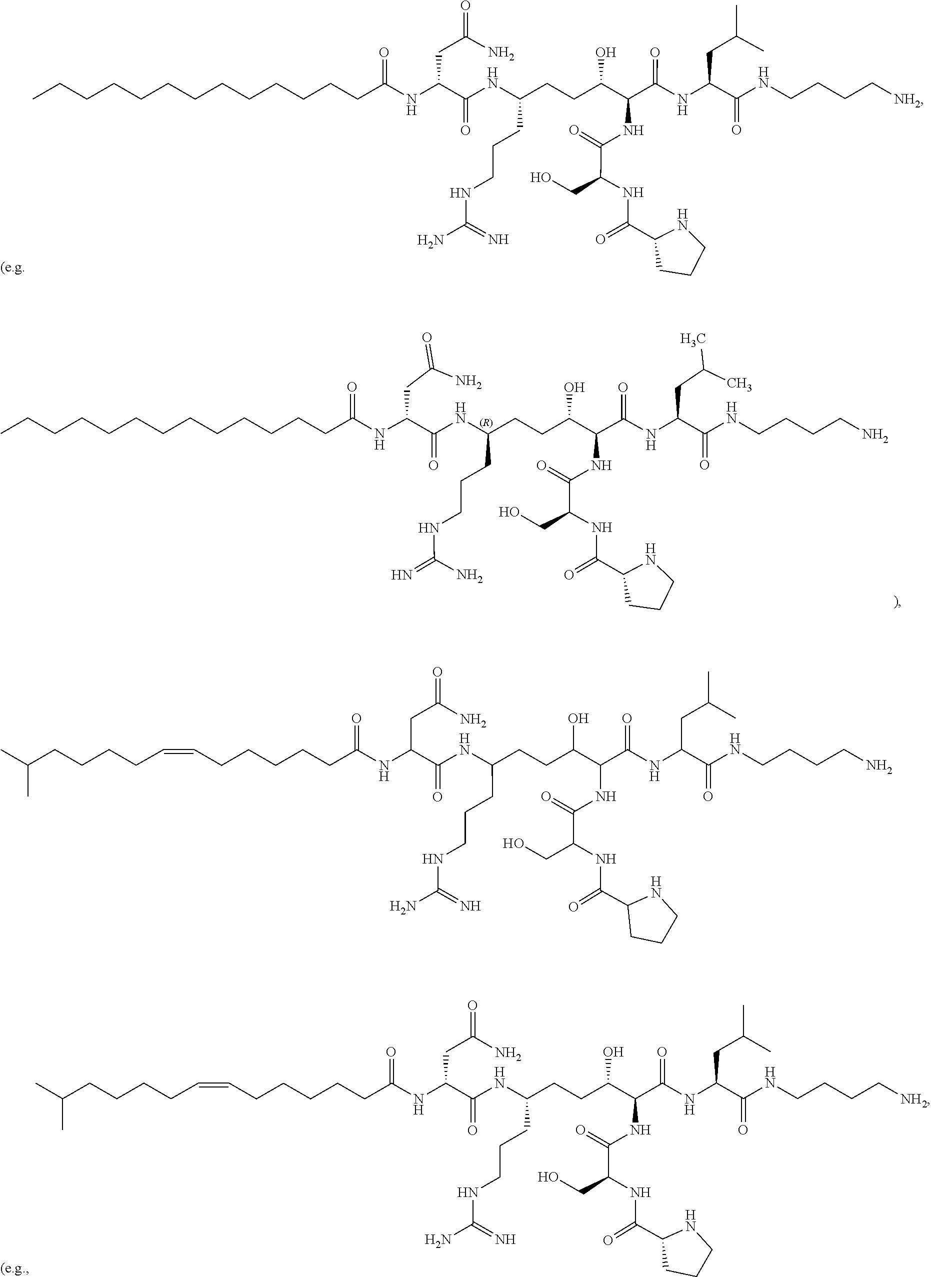

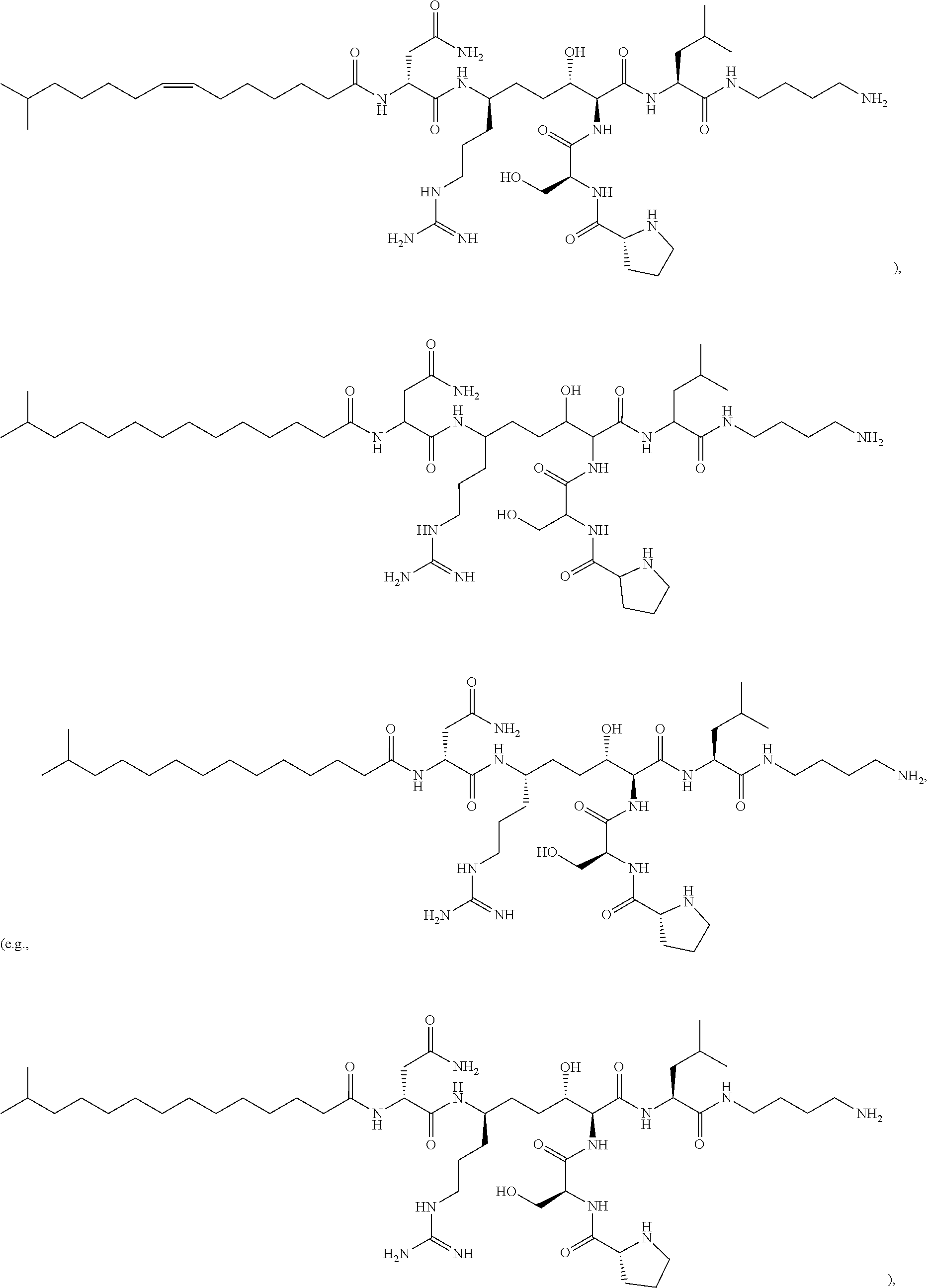

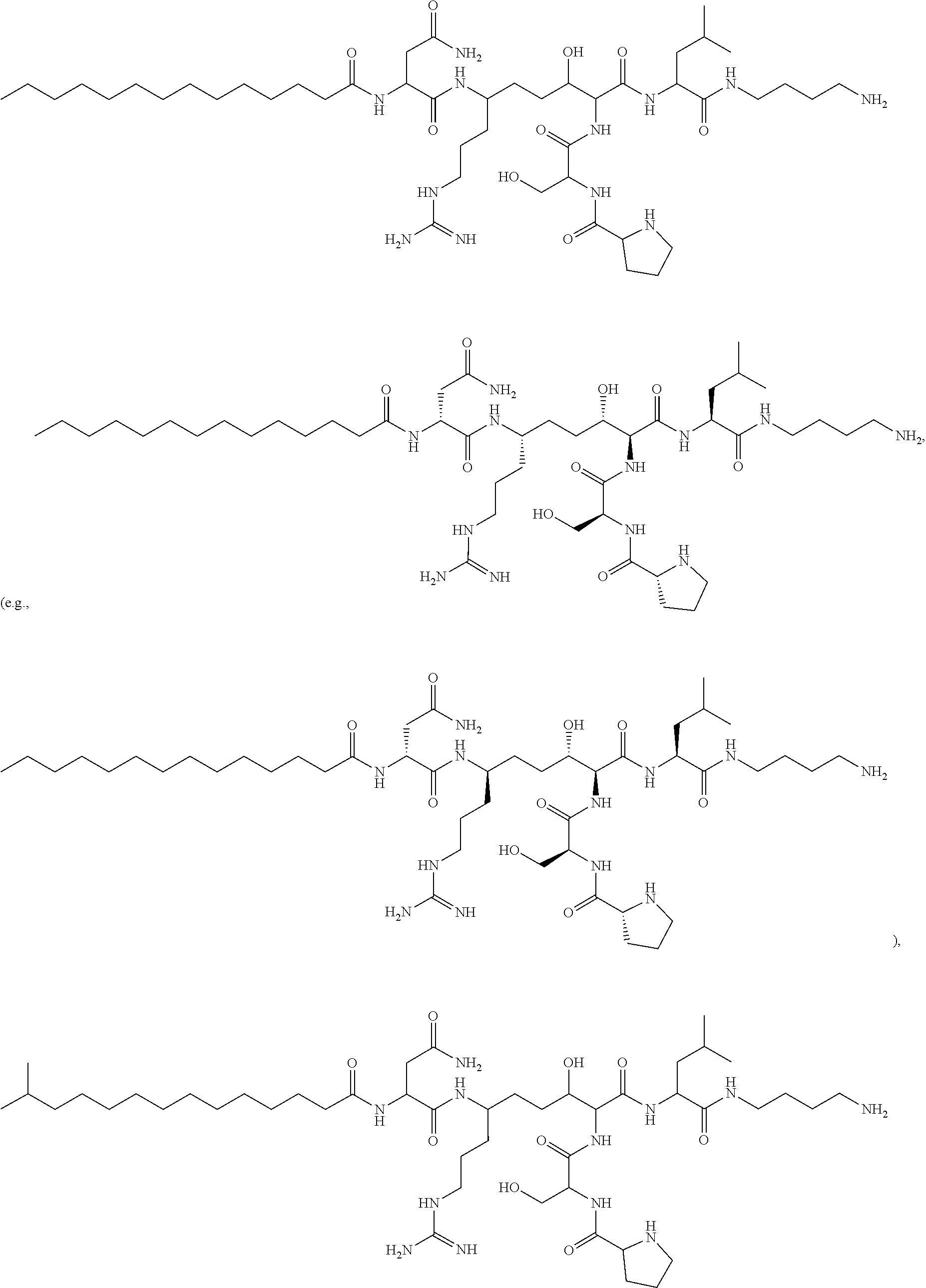 [SMILES: CCCCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN ; CCCCCCCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CCCCCCCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN ; CC(C)CCCCCCCCCCCC(=O)NC(CC(N)=O)C(=O)NC(CCCNC(=N)N)CCC(O)C(NC(=O)C(CO)NC(=O)C1CCCN1)C(=O)NC(CC(C)C)C(=O)NCCCCN]

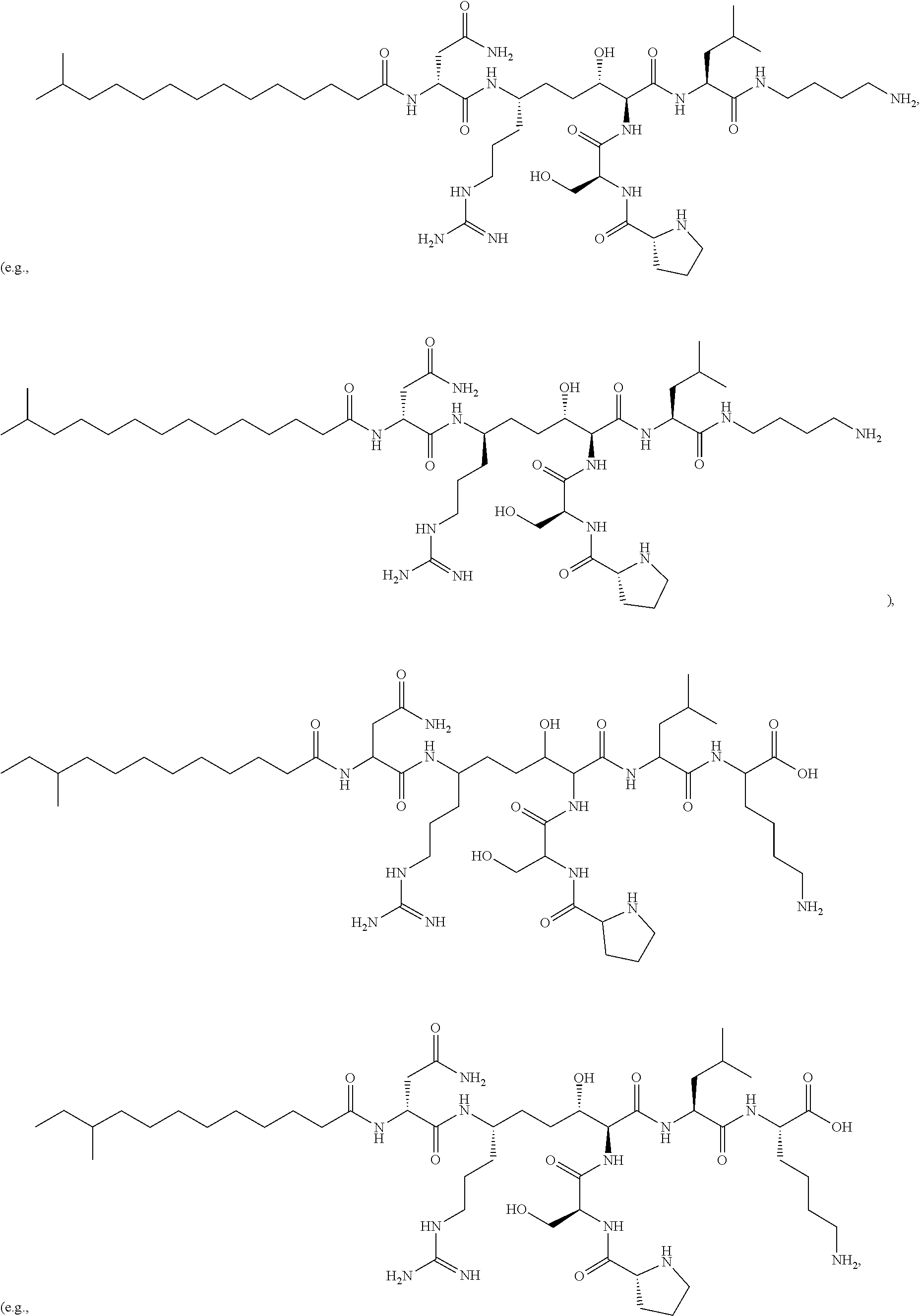

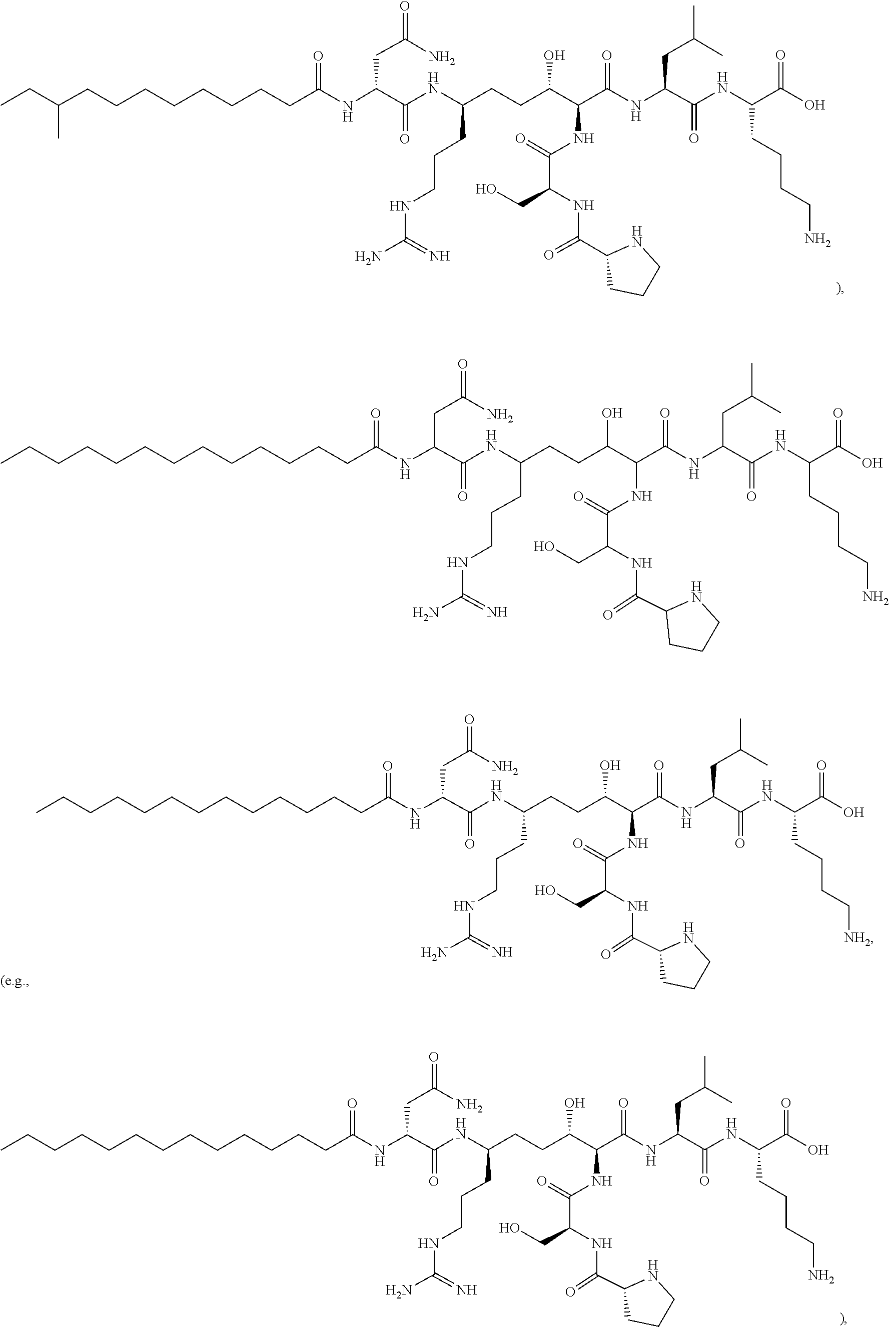

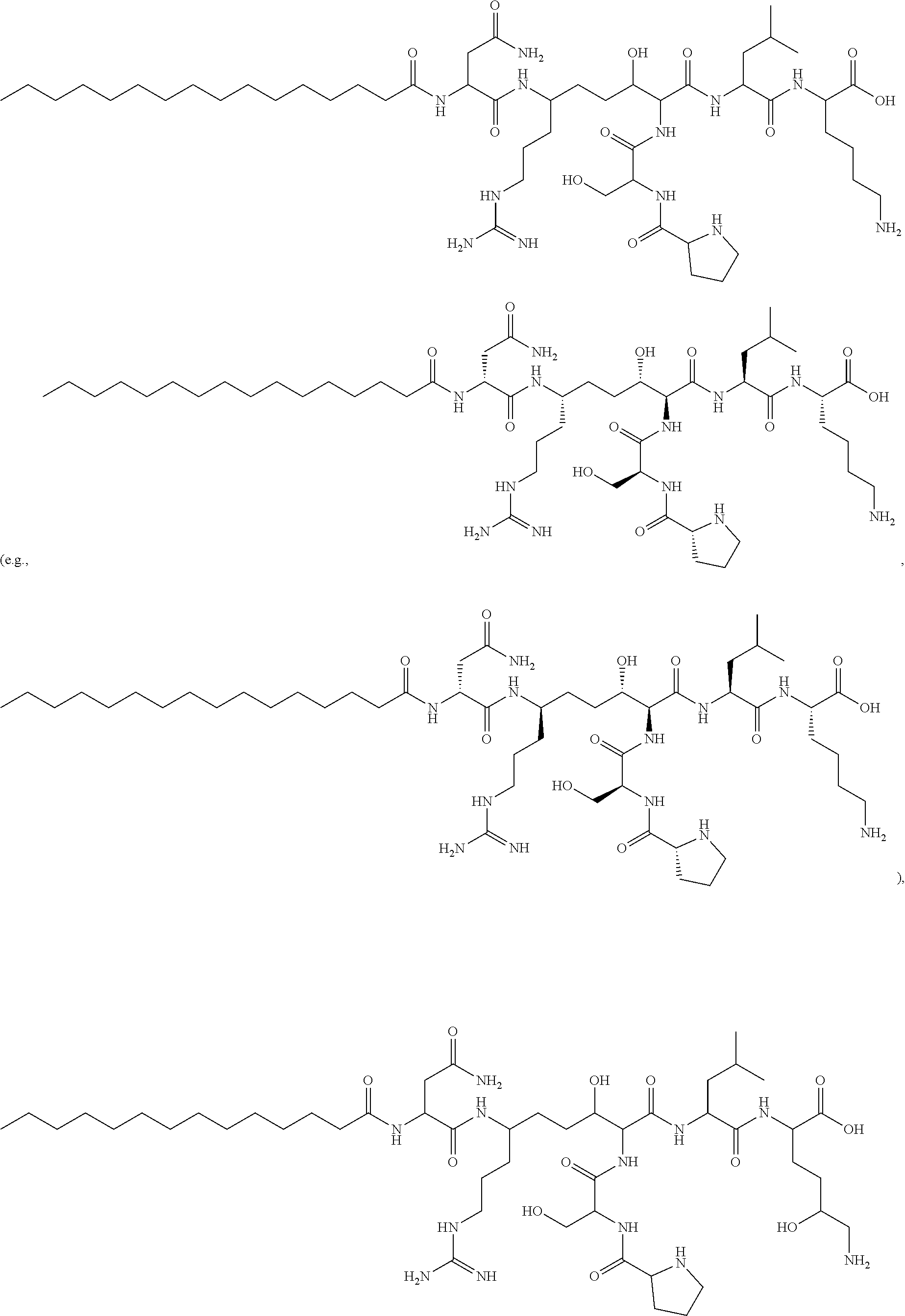

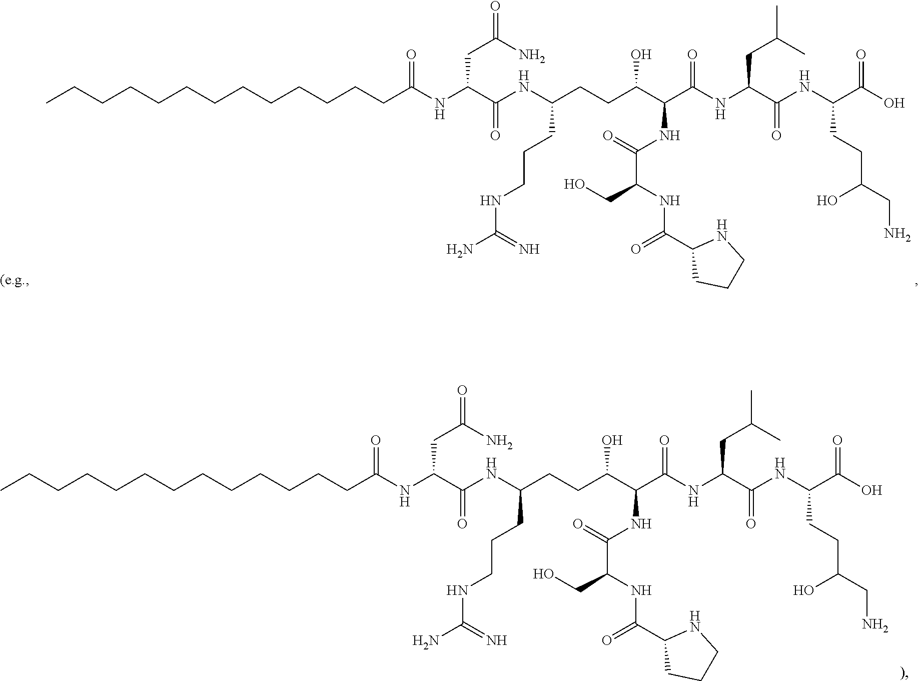 [SMILES: CCCCCCCCCCCCCC(=O)N[C@H](CC(N)=O)C(=O)N[C@@H](CCCNC(=N)N)CC[C@H](O)[C@H](NC(=O)[C@H](CO)NC(=O)[C@H]1CCCN1)C(=O)N[C@@H](CC(C)C)C(=O)N[C@@H](CCC(O)CN)C(=O)O ; CCCCCCCCCCCCCC(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)N[C@@H](CCC(O)CN)C(=O)O]

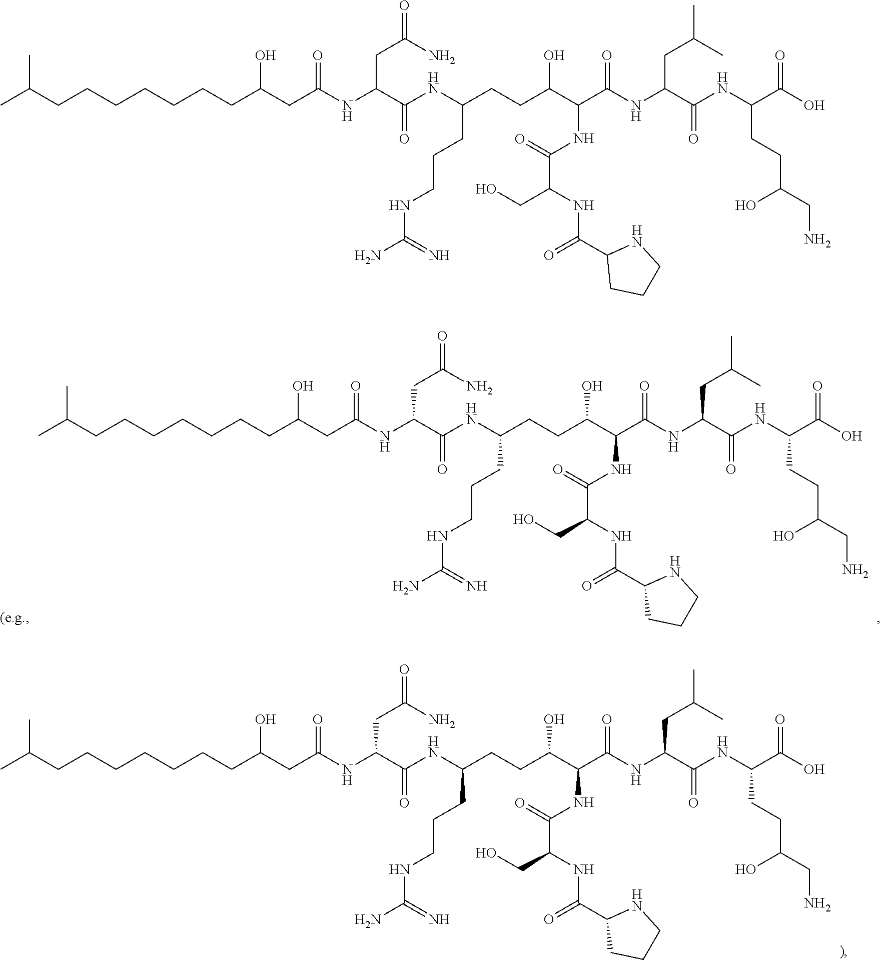

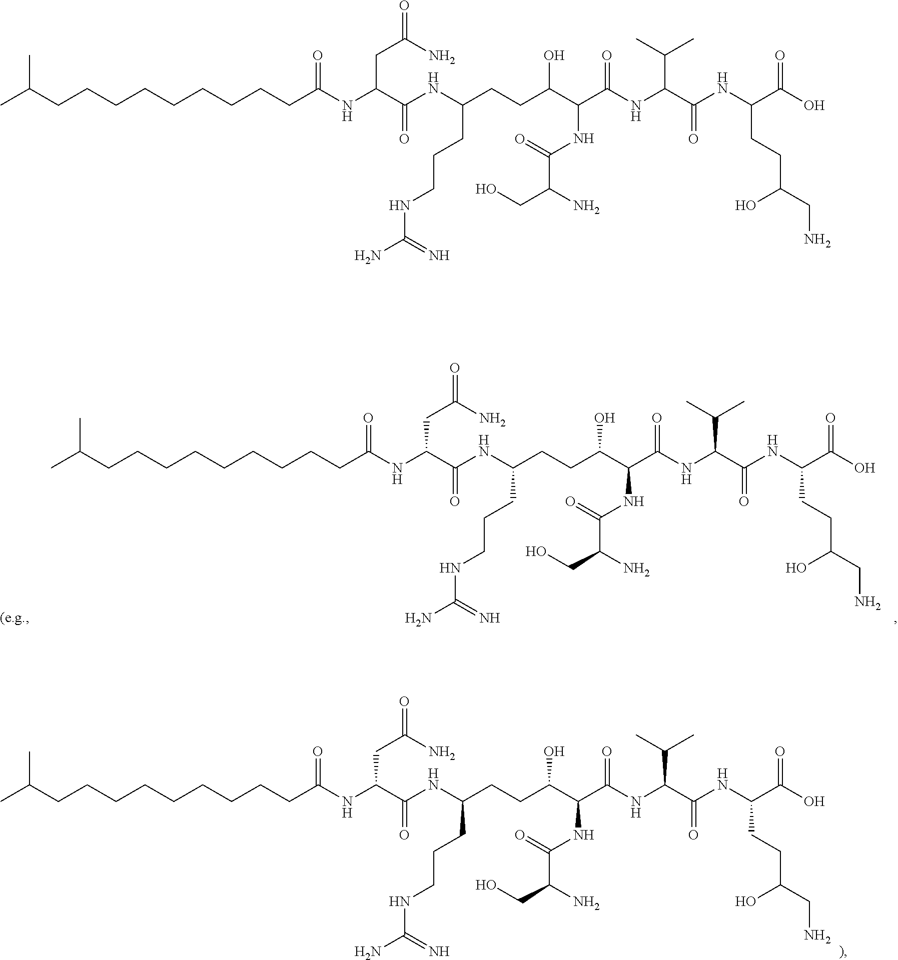 [SMILES: CC(C)CCCCCCCCCC(=O)NC(CC(N)=O)C(=O)NC(CCCNC(=N)N)CCC(O)C(NC(=O)C(N)CO)C(=O)NC(C(=O)NC(CCC(O)CN)C(=O)O)C(C)C ; CC(C)CCCCCCCCCC(=O)N[C@H](CC(N)=O)C(=O)N[C@@H](CCCNC(=N)N)CC[C@H](O)[C@H](NC(=O)[C@@H](N)CO)C(=O)N[C@H](C(=O)N[C@@H](CCC(O)CN)C(=O)O)C(C)C ; CC(C)CCCCCCCCCC(=O)N[C@H](CC(N)=O)C(=O)N[C@H](CCCNC(=N)N)CC[C@H](O)[C@H](NC(=O)[C@@H](N)CO)C(=O)N[C@H](C(=O)N[C@@H](CCC(O)CN)C(=O)O)C(C)C]

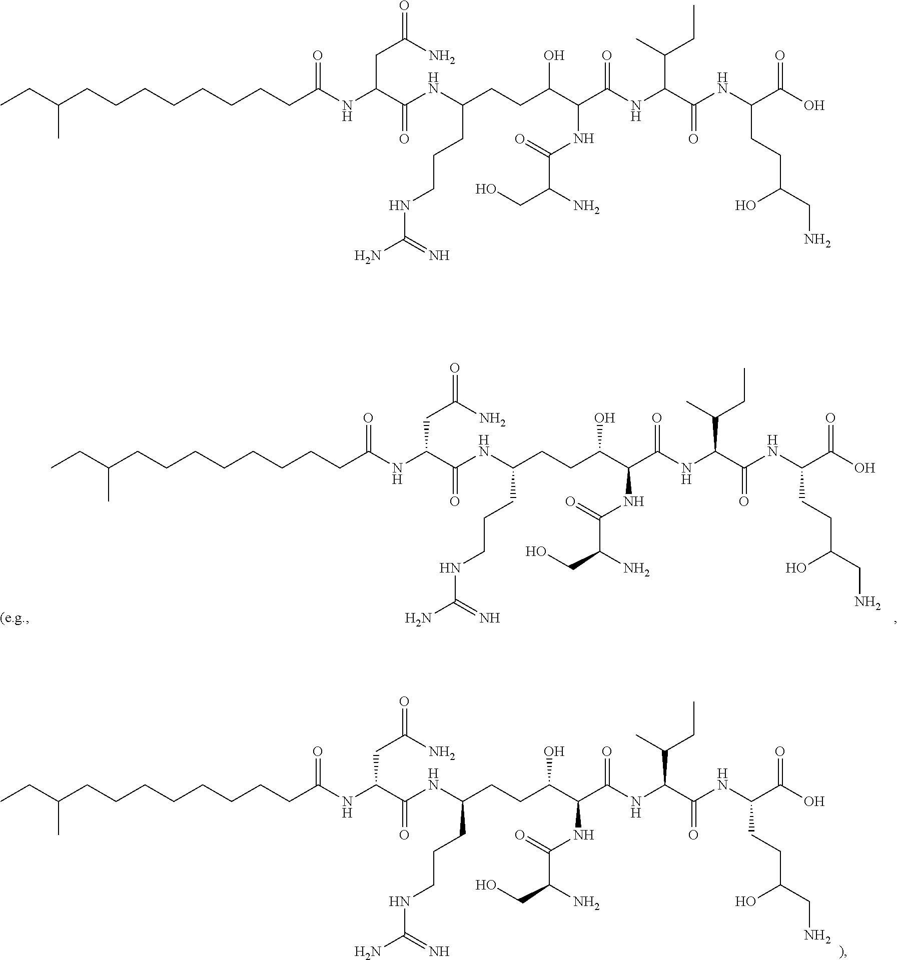 [SMILES: CCC(C)CCCCCCCCC(=O)NC(CC(N)=O)C(=O)NC(CCCNC(=N)N)CCC(O)C(NC(=O)C(N)CO)C(=O)NC(C(=O)NC(CCC(O)CN)C(=O)O)C(C)CC ; CCC(C)CCCCCCCCC(=O)N[C@H](CC(N)=O)C(=O)N[C@@H](CCCNC(=N)N)CC[C@H](O)[C@H](NC(=O)[C@@H](N)CO)C(=O)N[C@H](C(=O)N[C@@H](CCC(O)CN)C(=O)O)C(C)CC ; CCC(C)CCCCCCCCC(=O)N[C@H](CC(N)=O)C(=O)N[C@H](CCCNC(=N)N)CC[C@H](O)[C@H](NC(=O)[C@@H](N)CO)C(=O)N[C@H](C(=O)N[C@@H](CCC(O)CN)C(=O)O)C(C)CC]

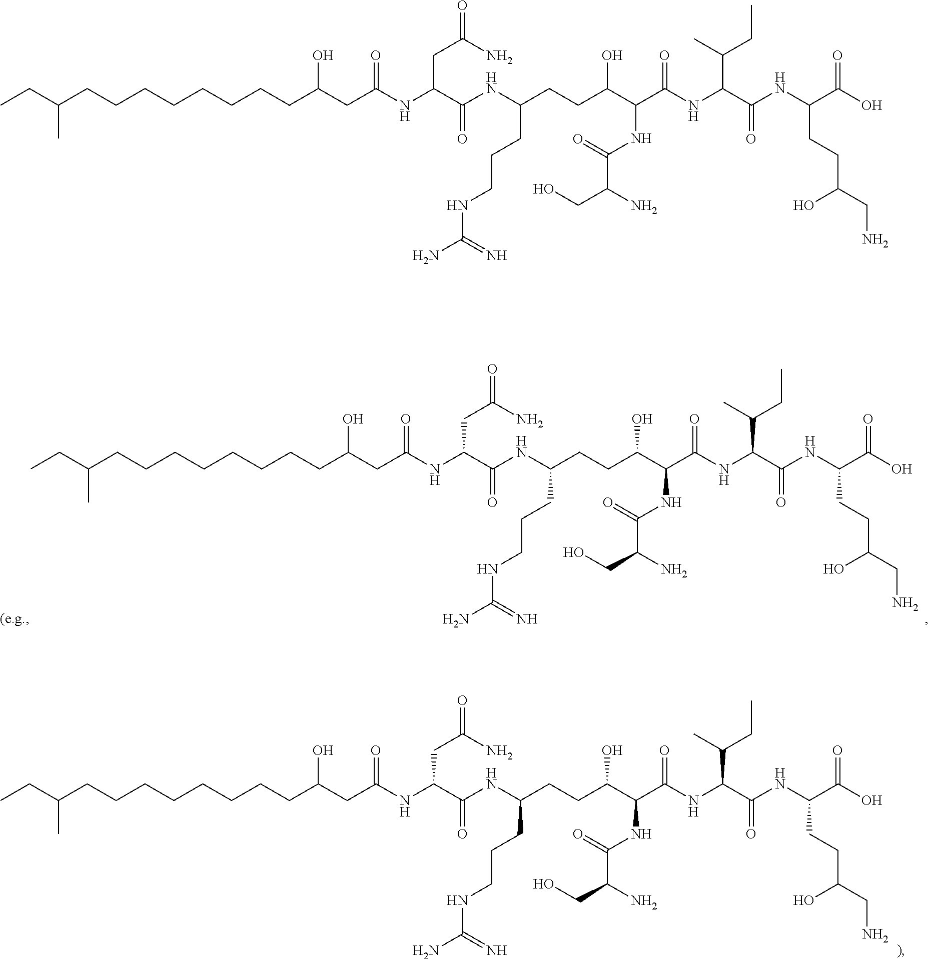 [SMILES: CCC(C)CCCCCCCCC(O)CC(=O)NC(CC(N)=O)C(=O)NC(CCCNC(=N)N)CCC(O)C(NC(=O)C(N)CO)C(=O)NC(C(=O)NC(CCC(O)CN)C(=O)O)C(C)CC ; CCC(C)CCCCCCCCC(O)CC(=O)N[C@H](CC(N)=O)C(=O)N[C@@H](CCCNC(=N)N)CC[C@H](O)[C@H](NC(=O)[C@@H](N)CO)C(=O)N[C@H](C(=O)N[C@@H](CCC(O)CN)C(=O)O)C(C)CC ; CCC(C)CCCCCCCCC(O)CC(=O)N[C@H](CC(N)=O)C(=O)N[C@H](CCCNC(=N)N)CC[C@H](O)[C@H](NC(=O)[C@@H](N)CO)C(=O)N[C@H](C(=O)N[C@@H](CCC(O)CN)C(=O)O)C(C)CC]

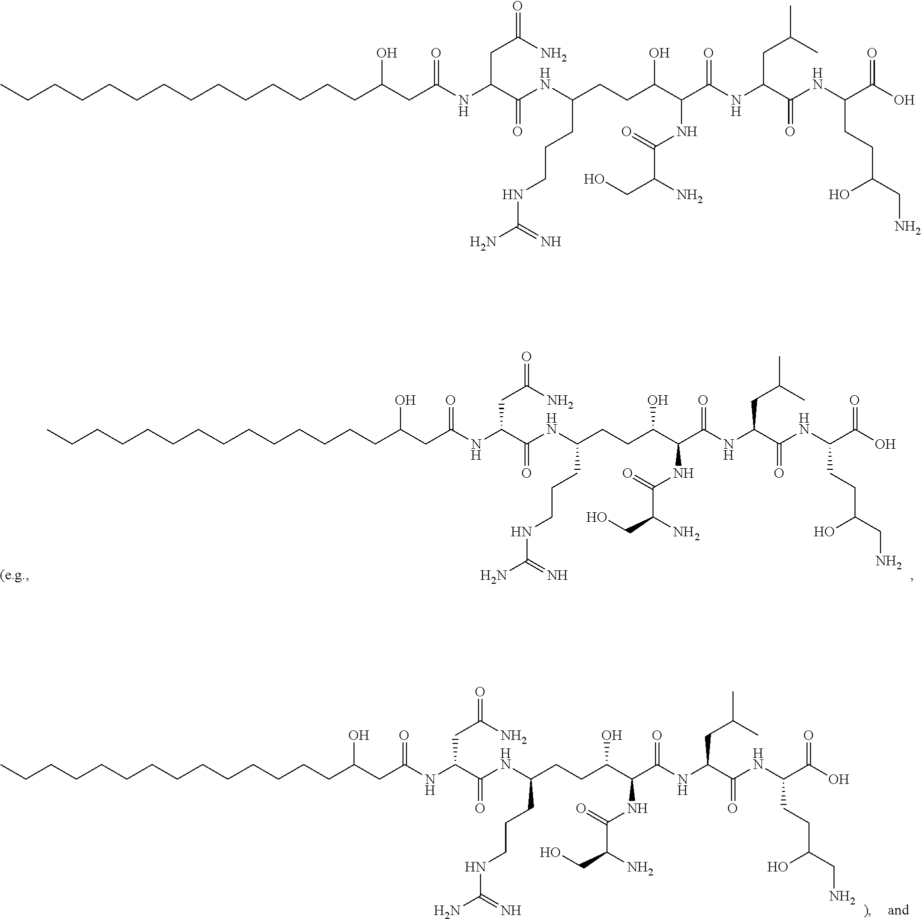 [SMILES: CCCCCCCCCCCCCCC(O)CC(=O)NC(CC(N)=O)C(=O)NC(CCCNC(=N)N)CCC(O)C(NC(=O)C(N)CO)C(=O)NC(CC(C)C)C(=O)NC(CCC(O)CN)C(=O)O ; CCCCCCCCCCCCCCC(O)CC(=O)N[C@H](CC(N)=O)C(=O)N[C@@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O ; CCCCCCCCCCCCCCC(O)CC(=O)N[C@H](CC(N)=O)C(=O)N[C@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O]

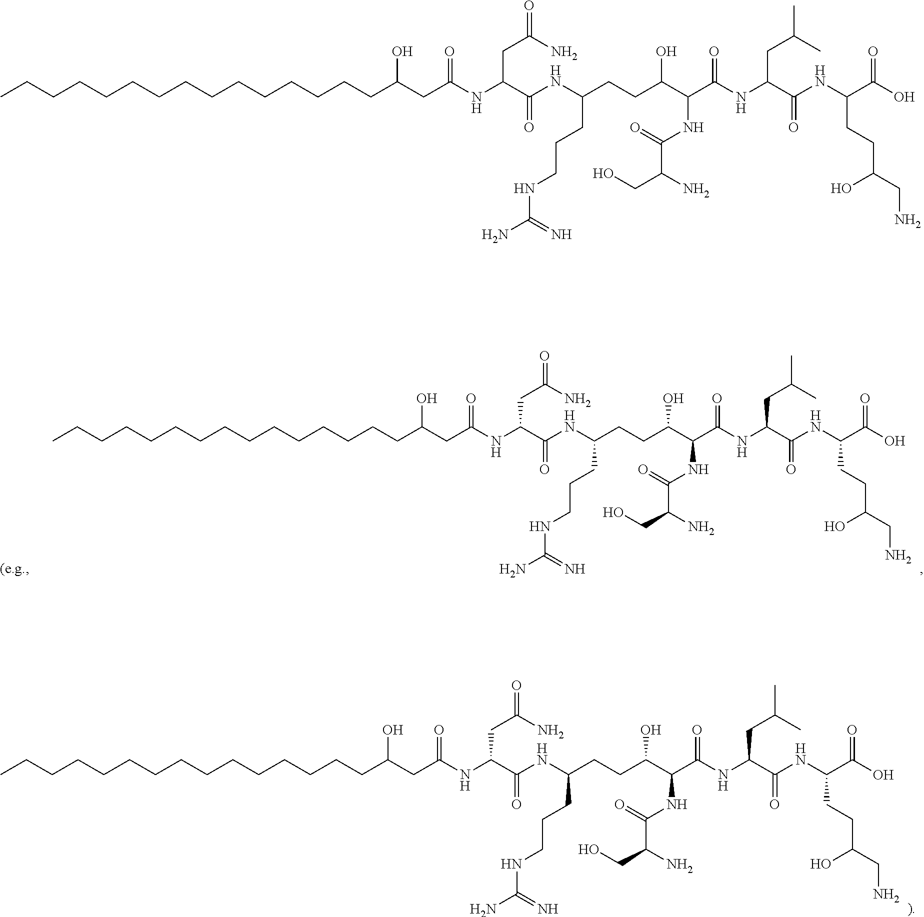 [SMILES: CCCCCCCCCCCCCCCC(O)CC(=O)NC(CC(N)=O)C(=O)NC(CCCNC(=N)N)CCC(O)C(NC(=O)C(N)CO)C(=O)NC(CC(C)C)C(=O)NC(CCC(O)CN)C(=O)O ; CCCCCCCCCCCCCCCC(O)CC(=O)N[C@H](CC(N)=O)C(=O)N[C@@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O ; CCCCCCCCCCCCCCCC(O)CC(=O)N[C@H](CC(N)=O)C(=O)N[C@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O]

The present disclosure includes all possible stereoisomers and geometric isomers of a compound of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of a compound of the present disclosure are possible, the present disclosure is intended to include all tautomeric forms of the compounds.

Compounds of the disclosure may exist as salts. Pharmaceutically acceptable salts of the compounds of the disclosure generally are preferred in the methods of the disclosure. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, hydrates, or prodrugs thereof.

The compounds may exhibit wide variability in pharmacokinetic and physicochemical properties while still retaining desirable biological activity as described herein. For example, solubility, e.g., log P, is variable while still retaining desirable biological activity as described herein.

In an aspect, the present disclosure provides compositions comprising one or more compound(s) of the present disclosure. The compositions may further comprise one or more pharmaceutically acceptable carrier(s).

The compositions may include one or more pharmaceutically acceptable carrier(s). The composition may be for administration to a subject in need of treatment.

Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, such as, for example, sodium carboxymethyl cellulose, ethyl cellulose, and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example, glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2012) 22nd Edition, Philadelphia, PA. Lippincott Williams & Wilkins. In various examples, the composition may be suitable for injection. Parenteral administration includes infusions and injections, such as, for example, intramuscular, intravenous, intraarterial, intraperitoneal, subcutaneous administration, and the like.

The compositions may be administered systemically. Compositions may be administered orally, may be administered parenterally, and/or intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

The compositions of the present disclosure may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Preferably, the compositions are administered orally, intraperitoneally, or intravenously.

Compositions of the disclosure can comprise more than one pharmaceutical agent. For example, a first composition comprising a compound of the disclosure and a first pharmaceutical agent can be separately prepared from a composition which comprises the same compound of the disclosure and a second pharmaceutical agent, and such preparations can be mixed to provide a two-pronged (or more) approach to achieving the desired prophylaxis or therapy in an individual. Further, compositions of the disclosure can be prepared using mixed preparations of any of the compounds disclosed herein.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a compound of the present disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to a compound of the disclosure, the composition may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In one embodiment, the pharmaceutically-acceptable formulation is such that it provides sustained delivery of a compound of the present disclosure to an individual for at least 12 hours, 24 hours, 36 hours, 48 hours, one week, two weeks, three weeks, or four weeks after the pharmaceutically-acceptable formulation is administered to the individual.

The compositions may conveniently be presented in unit dosage form and may be prepared by any methods well known in the art of pharmacy. The amount of active ingredient which can be combined with a carrier material to produce a single dosage form will vary depending upon the individual being treated, the particular mode of administration. The amount of active ingredient which can be combined with a carrier material to produce a single dosage form will generally be that amount of a compound of the present disclosure which produces a therapeutic effect. Generally, out of one hundred percent, this amount will range from about 1 percent to about ninety-nine percent of active ingredient.

Methods of preparing these compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various examples of the present disclosure. They are not intended to be limiting in any way.

Statement 1. A method for selectively killing at least a portion of an E. coli population, wherein the population of E. coli expresses a ClbP, comprising contacting the E. coli population with a prodrug antibiotic, wherein the prodrug antibiotic is cleaved in the E. coli and a therapeutically effective amount of an antibiotic is formed and at least a portion of the E. coli population is killed.
Statement 2. A method according to Statement 1, wherein the prodrug antibiotic is chosen from

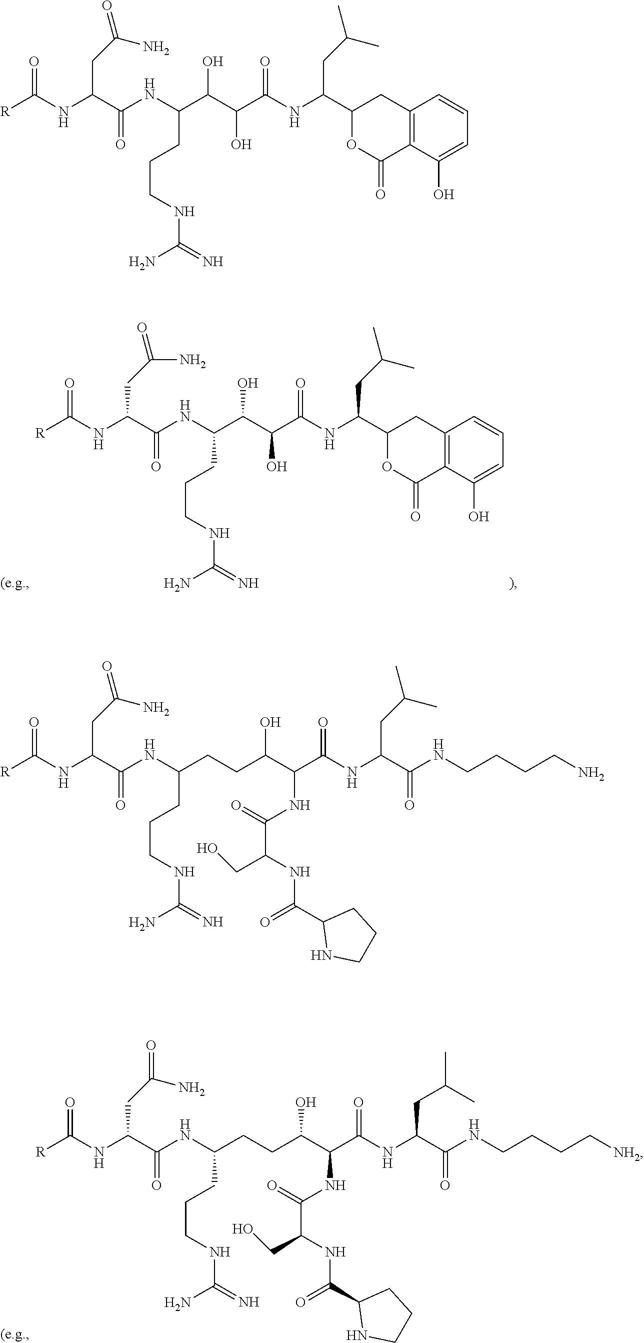

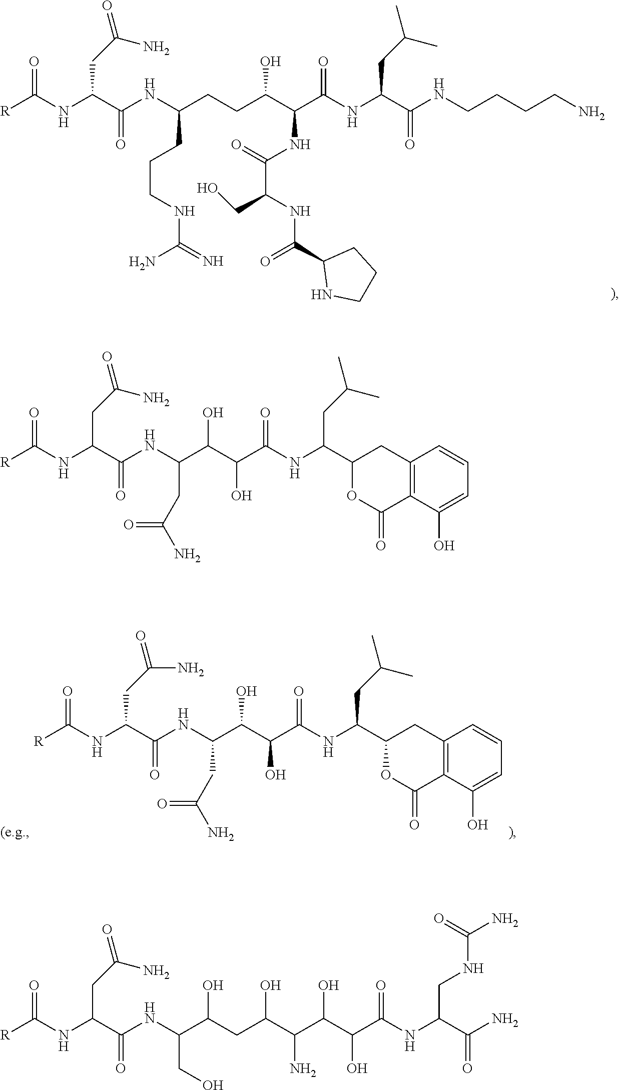

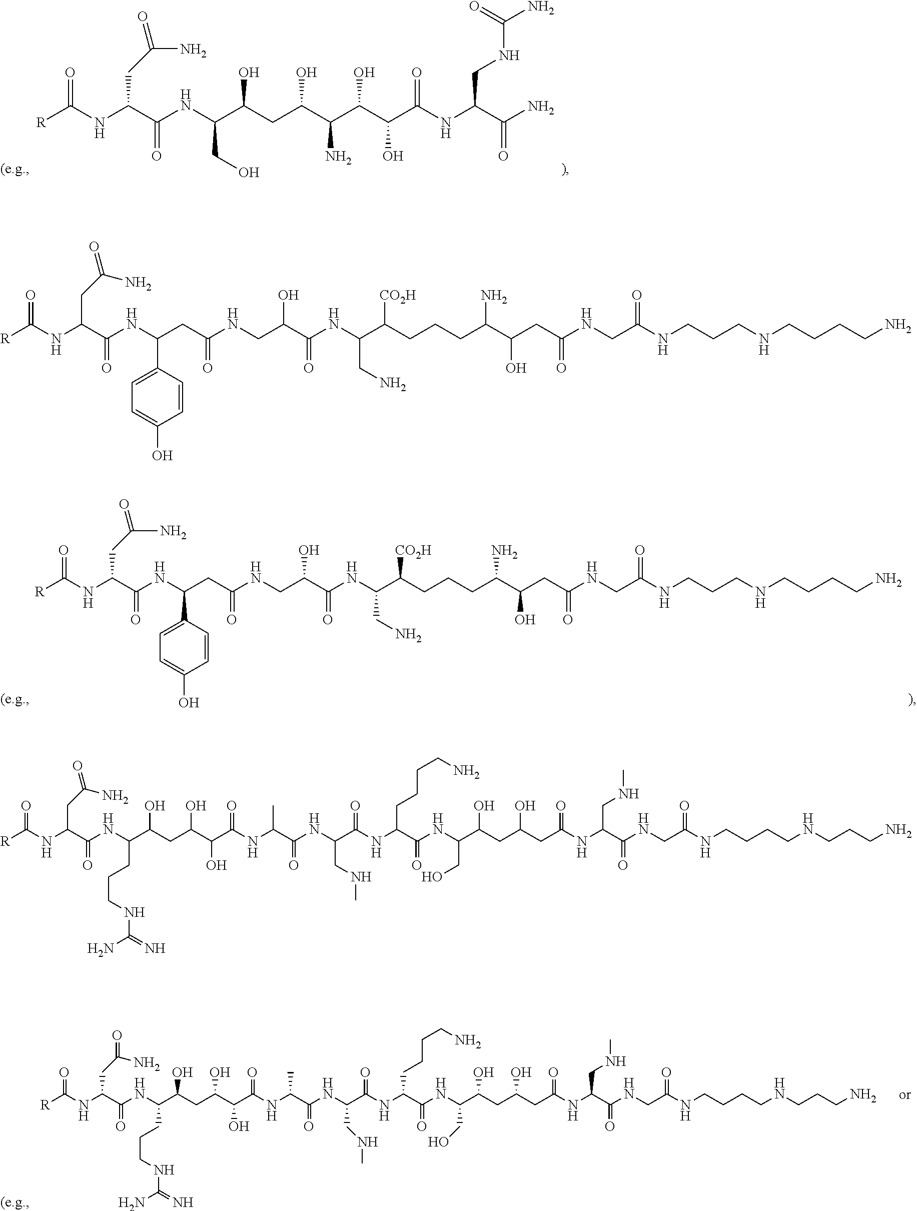

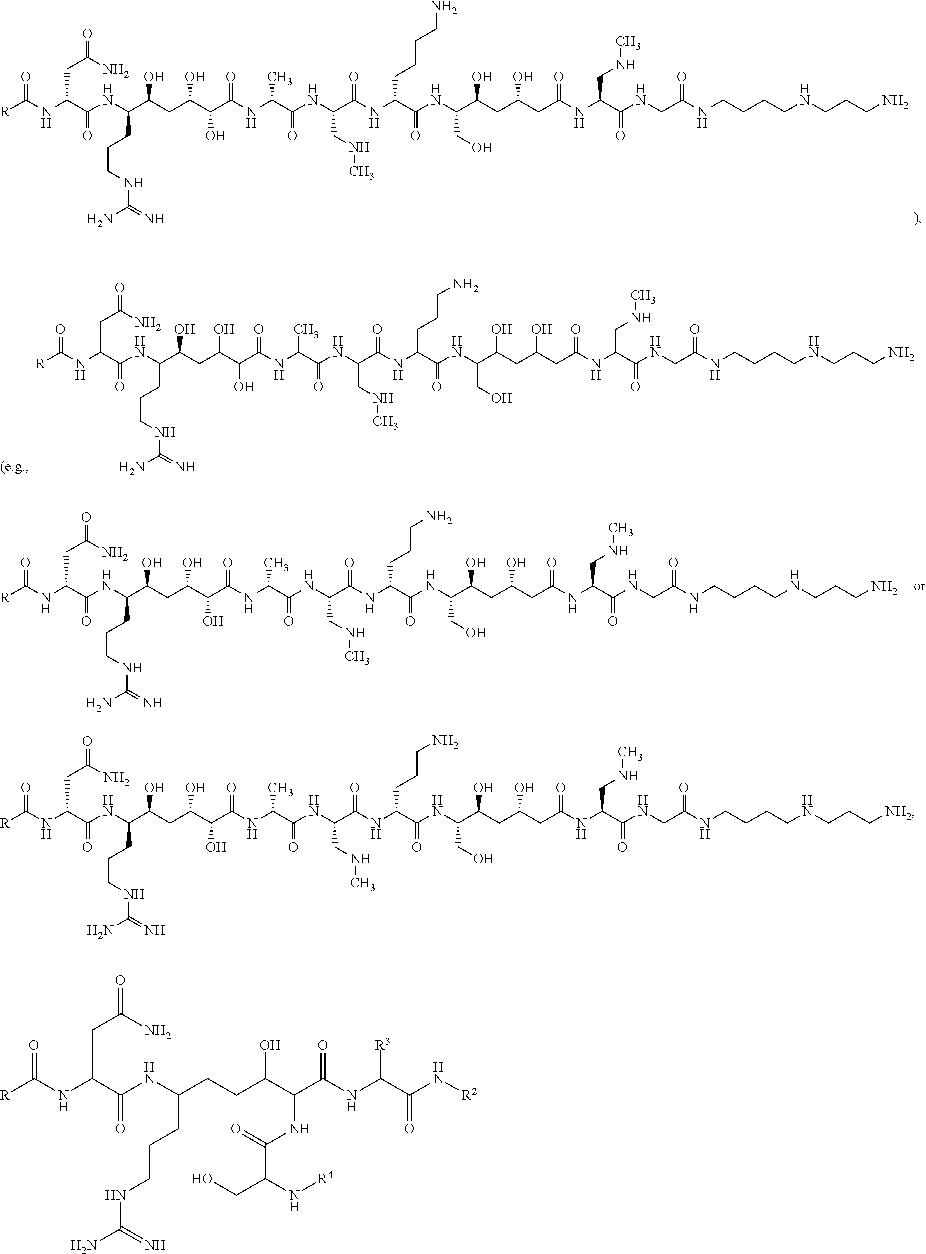

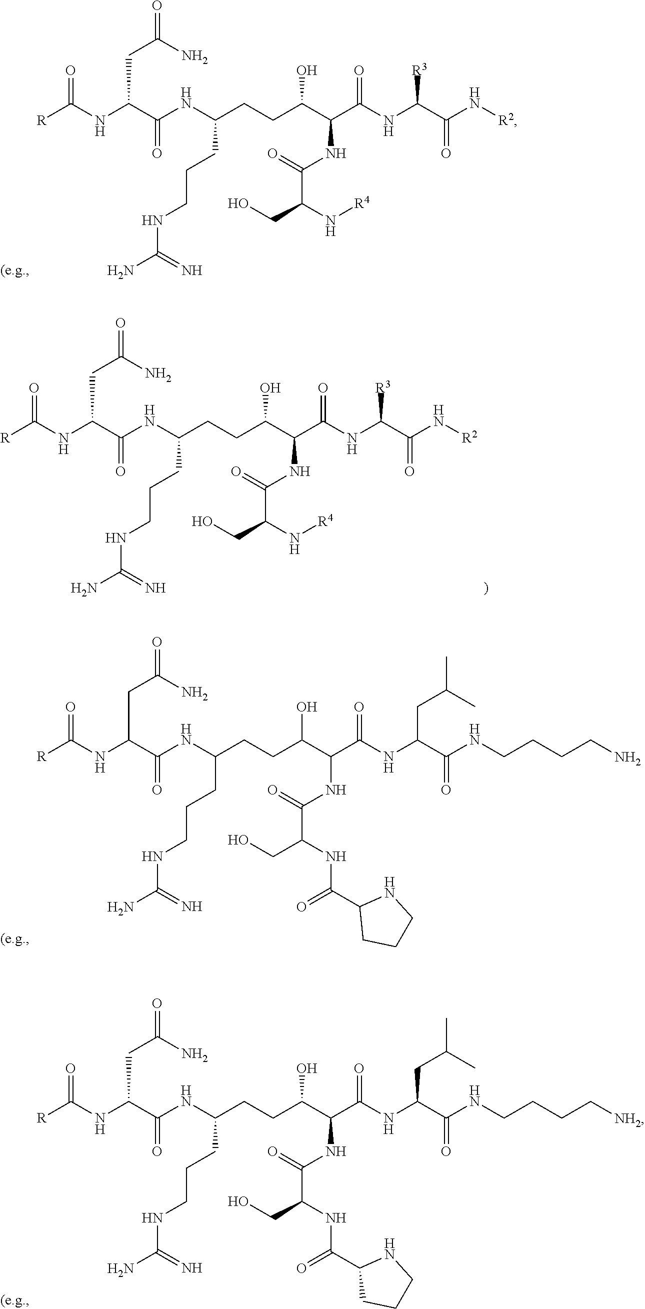

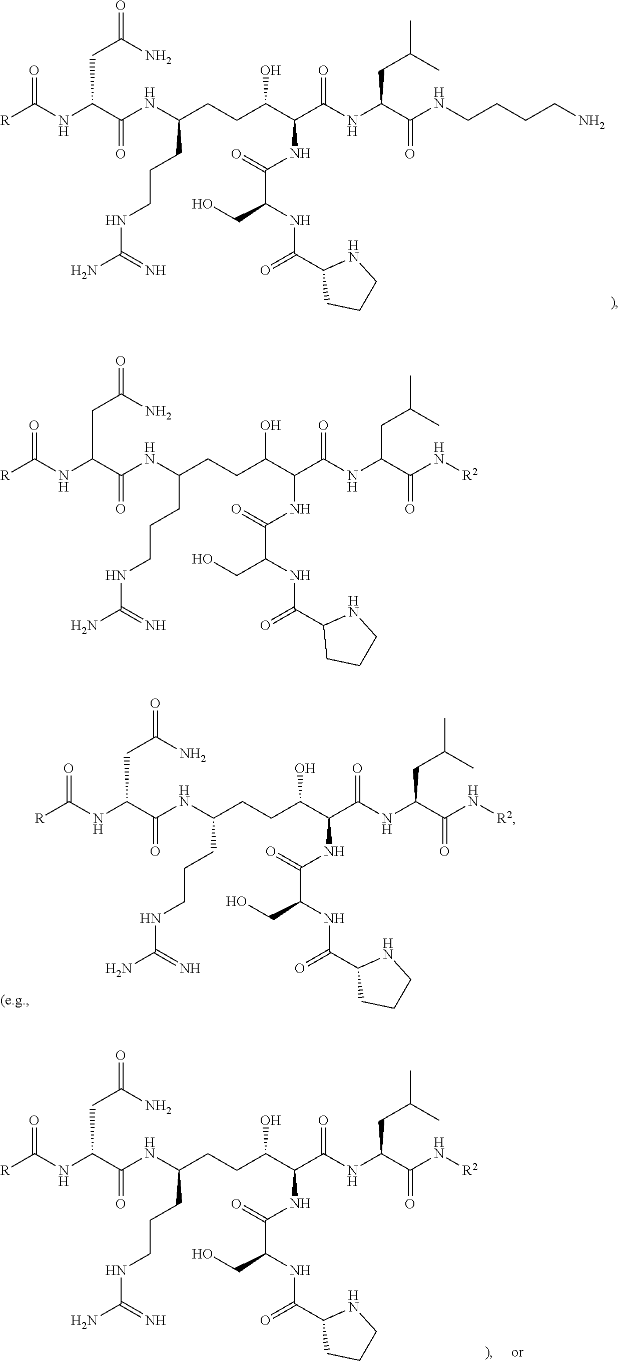

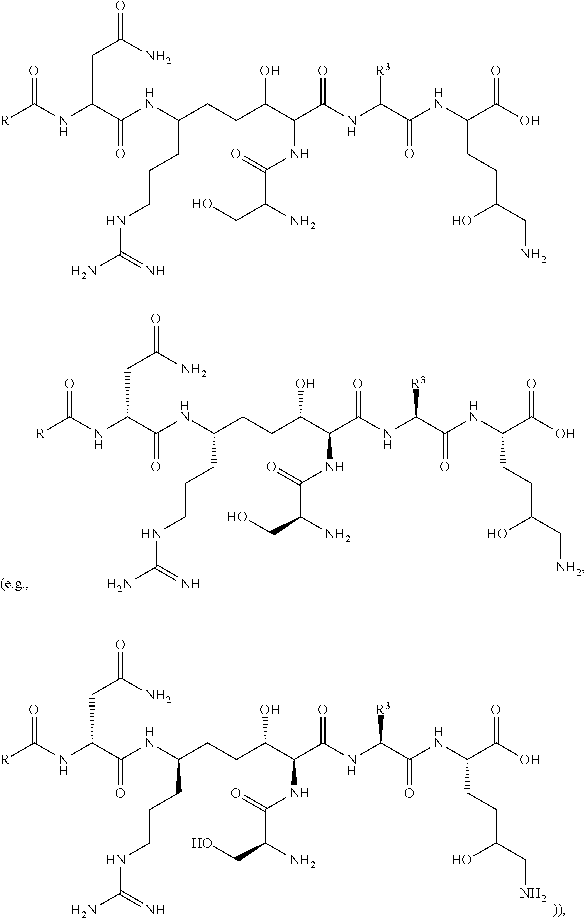

isomers, stereoisomers, enantiomers, diastereomers, salts, hydrates, and solvates of any one or more of the foregoing, and any combination of any of the foregoing, wherein R is a substituted or unsubstituted aliphatic group, wherein the aliphatic group is a linear substituted or unsubstituted aliphatic group or a branched substituted or unsubstituted aliphatic group; R² is an amino acid residue (e.g., a canonical or noncanonical amino acid residue), a substituted or unsubstituted aliphatic group, or H; R³ is a substituted or unsubstituted aliphatic group or an amino acid side chain; and R⁴ is a substituted or unsubstituted acyl group, an amino acid residue, or H.
Statement 3. A method according to Statement 2, wherein the aliphatic groups are 1 to 25 carbon atoms in length (e.g., 1, 2, 3, 4, 5, 6, 7, 8, 9, 10, 11, 12, 13, 14, 15, 16, 17, 18, 19, 20, 21, 22, 23, 24, or 25 carbon atoms in length).
Statement 4. A method according to Statement 2 or Statement 3, wherein the aliphatic groups have one or more aromatic rings, one or more pi bonds, one or more hydroxy groups, and/or one or more halogens.
Statement 5. A method according to Statement 4, wherein the aliphatic groups are substituted with one or more fluoro groups.
Statement 6. A method according to any one of Statements 2 to 5, wherein R has the following structure

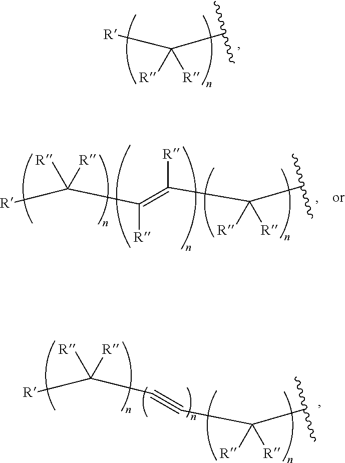

wherein R′ is hydrogen, a halogen (e.g., bromo, fluoro, or the like), an aryl group (e.g., phenyl, napthyl, or the like), a methyl group, halogenated methyl group (e.g., —CF₃), an amino group, or a hydroxy, or the like, and each R″ is independently hydrogen, a halogen (e.g., bromo, fluoro, or the like), an aryl group (e.g., phenyl, napthyl, or the like), a methyl group, halogenated methyl group (e.g., —CF₃), a hydroxy, or the like.
Statement 7. A method according to any one of Statements 2 to 5, wherein R has the following structure:

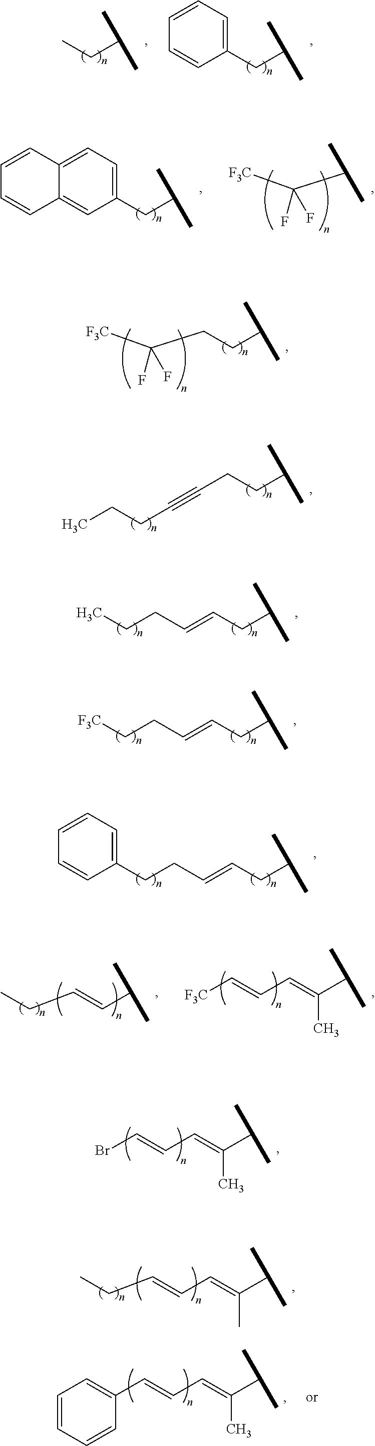

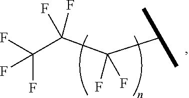

wherein each n is independently 0 to 18 (e.g., 0, 1, 2, 3, 4, 5, 6, 7, 8, 9, 10, 11, 12, 13, 14, 15, 16, 17, 18).
Statement 8. A method according to any one of Statements 2 to 5, wherein R has the following structure:

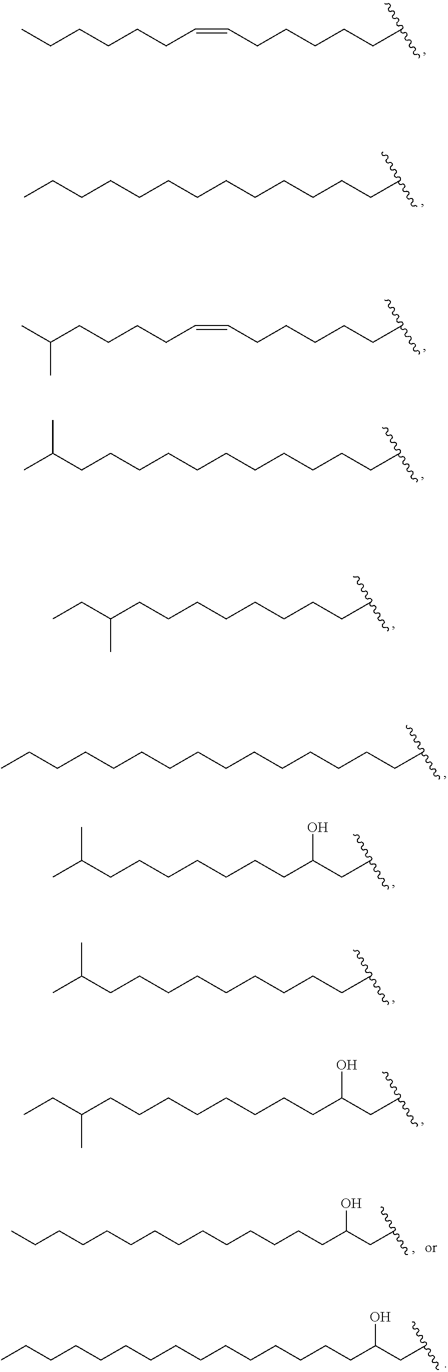

Statement 9. A method according to any one of Statements 2 to 8, wherein R² is

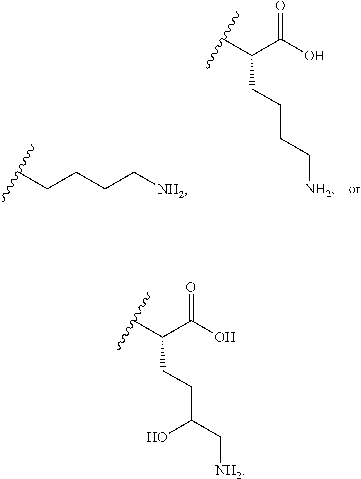 [SMILES: *CCCCN ; *[C@@H](CCCCN)C(=O)O ; *[C@@H](CCC(O)CN)C(=O)O]

Statement 10. A method according to any one of Statements 2 to 10, wherein R³ is

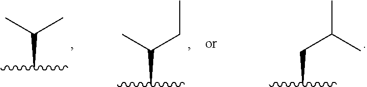

Statement 11. A method according to any one of Statements 2 to 10, wherein R⁴ is H or

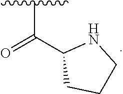 [SMILES: *C(=O)[C@H]1CCCN1]

Statement 12. A method according to any one of Statements 2 to 5, wherein the prodrug antibiotic has the following structure:

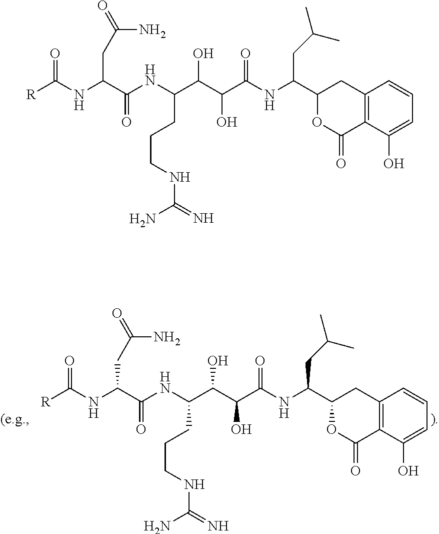

Statement 13. A method according to Statement 12, wherein the prodrug antibiotic is:

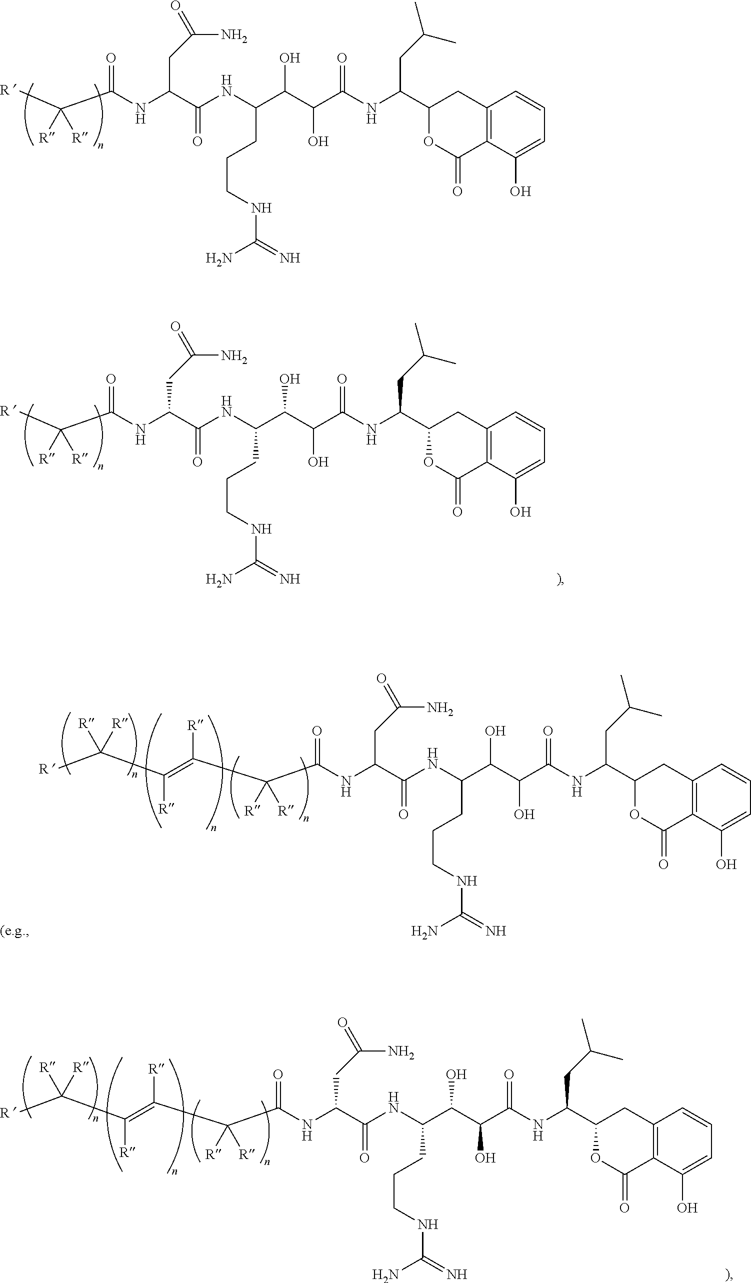

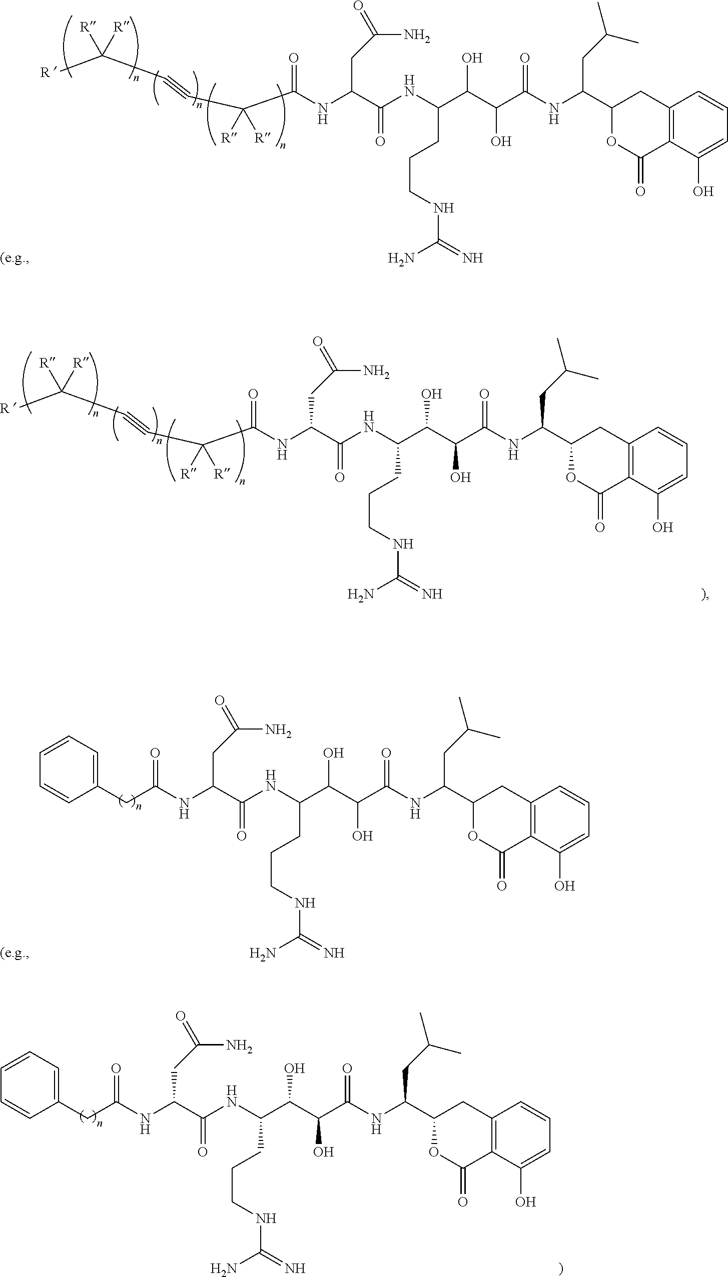

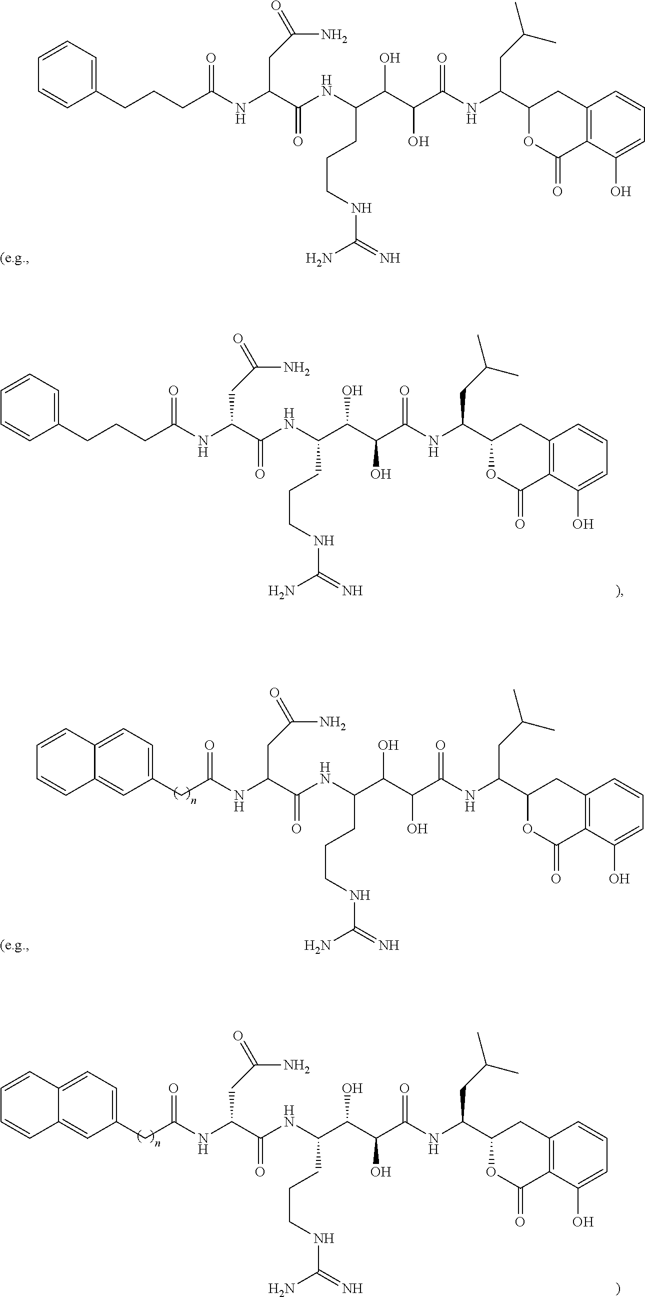

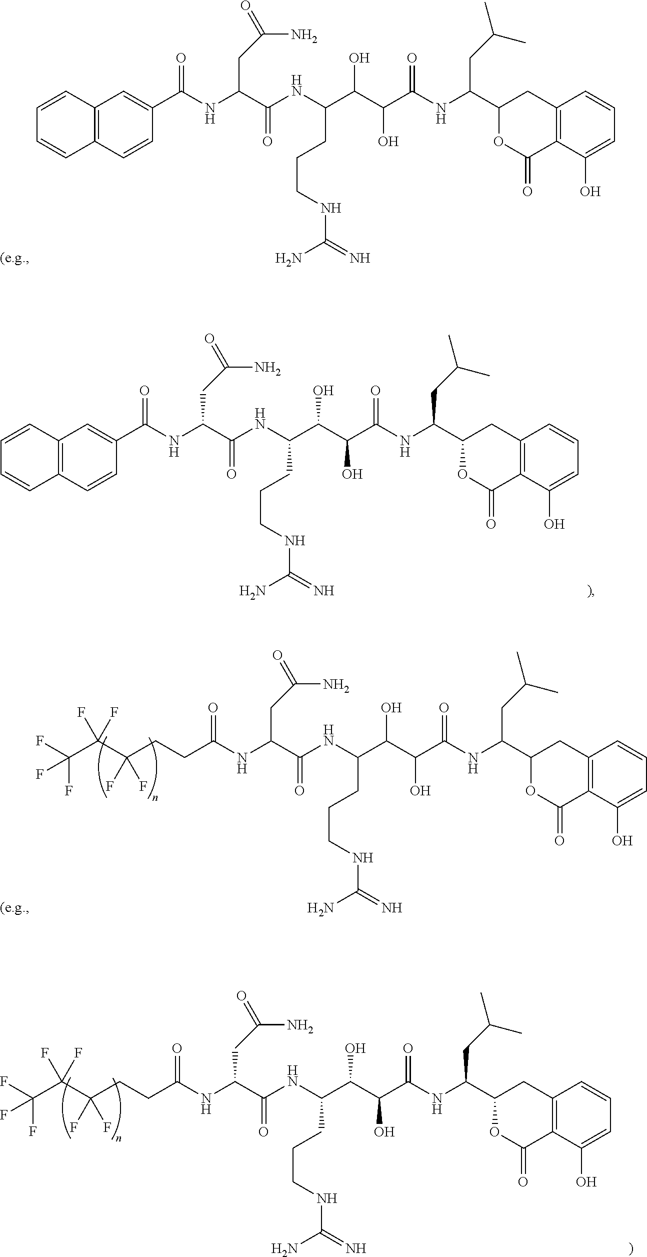

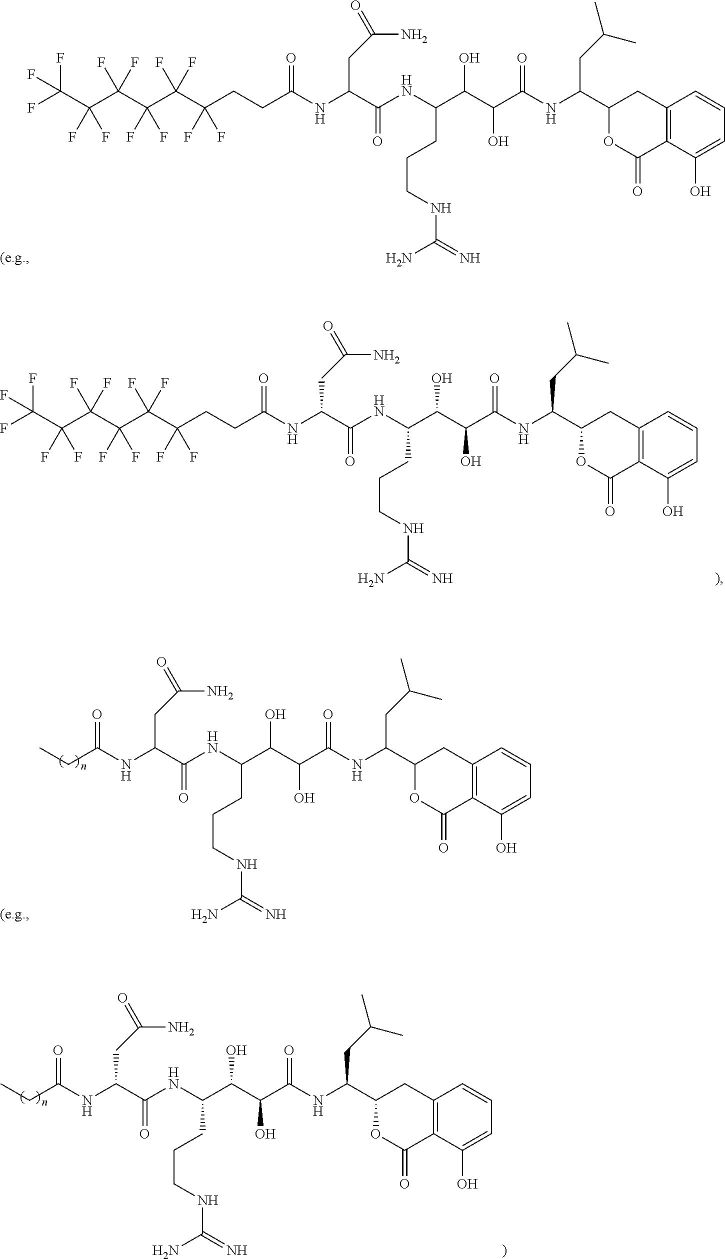

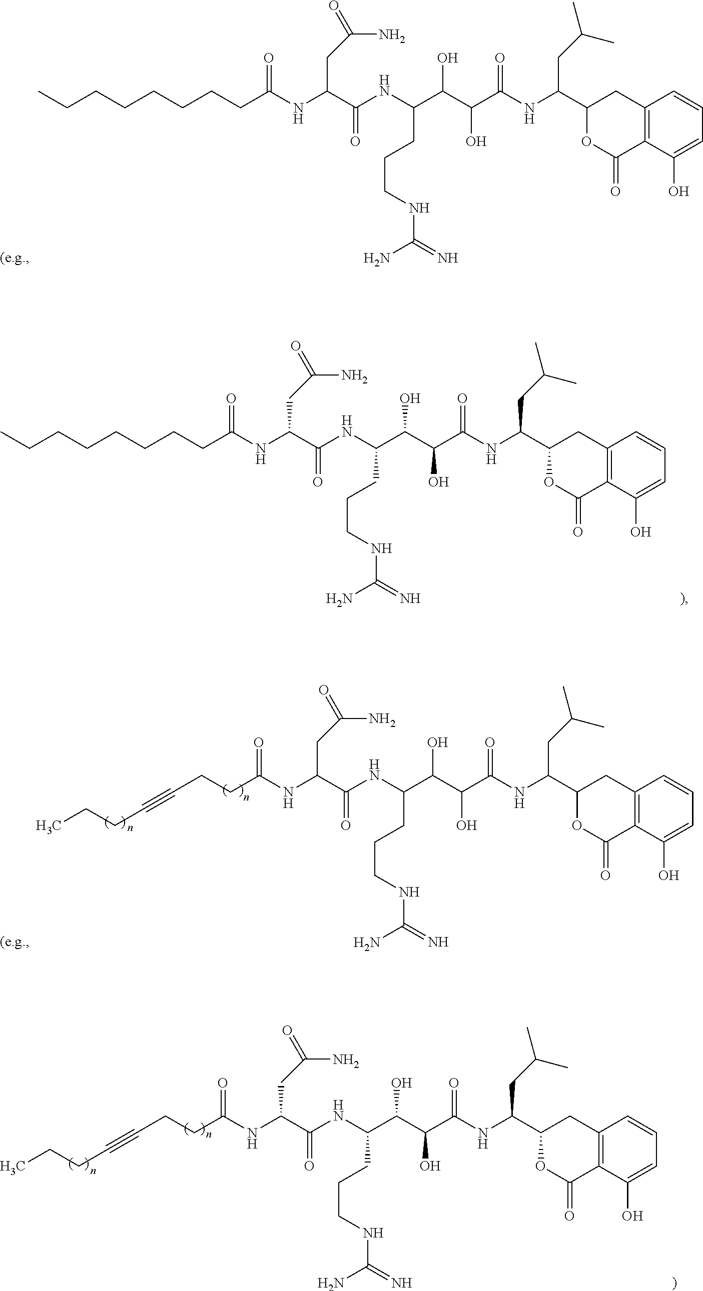

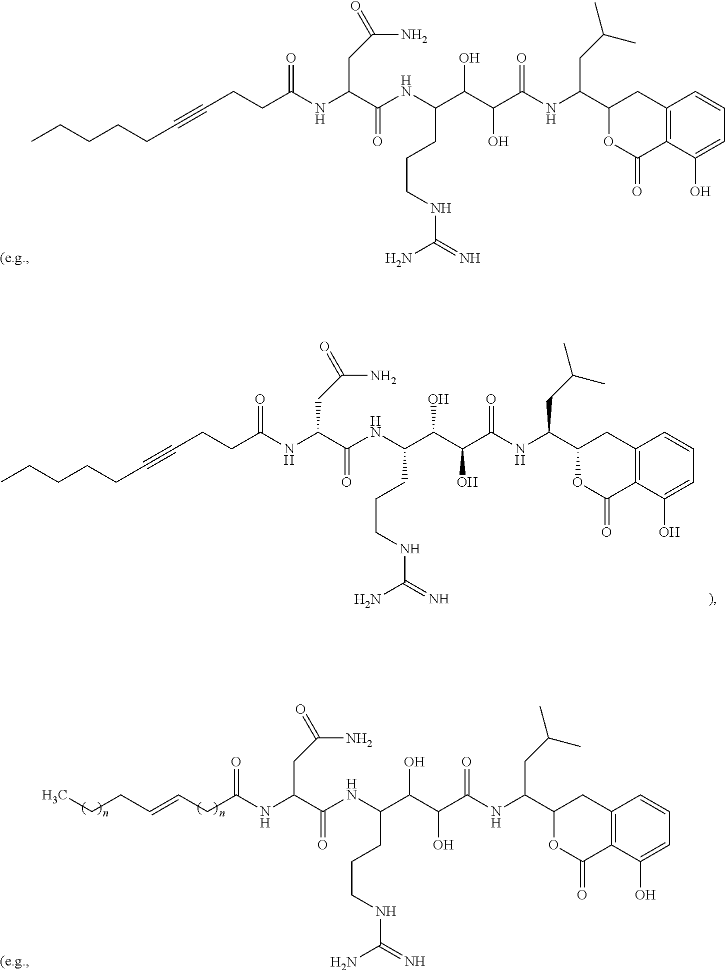

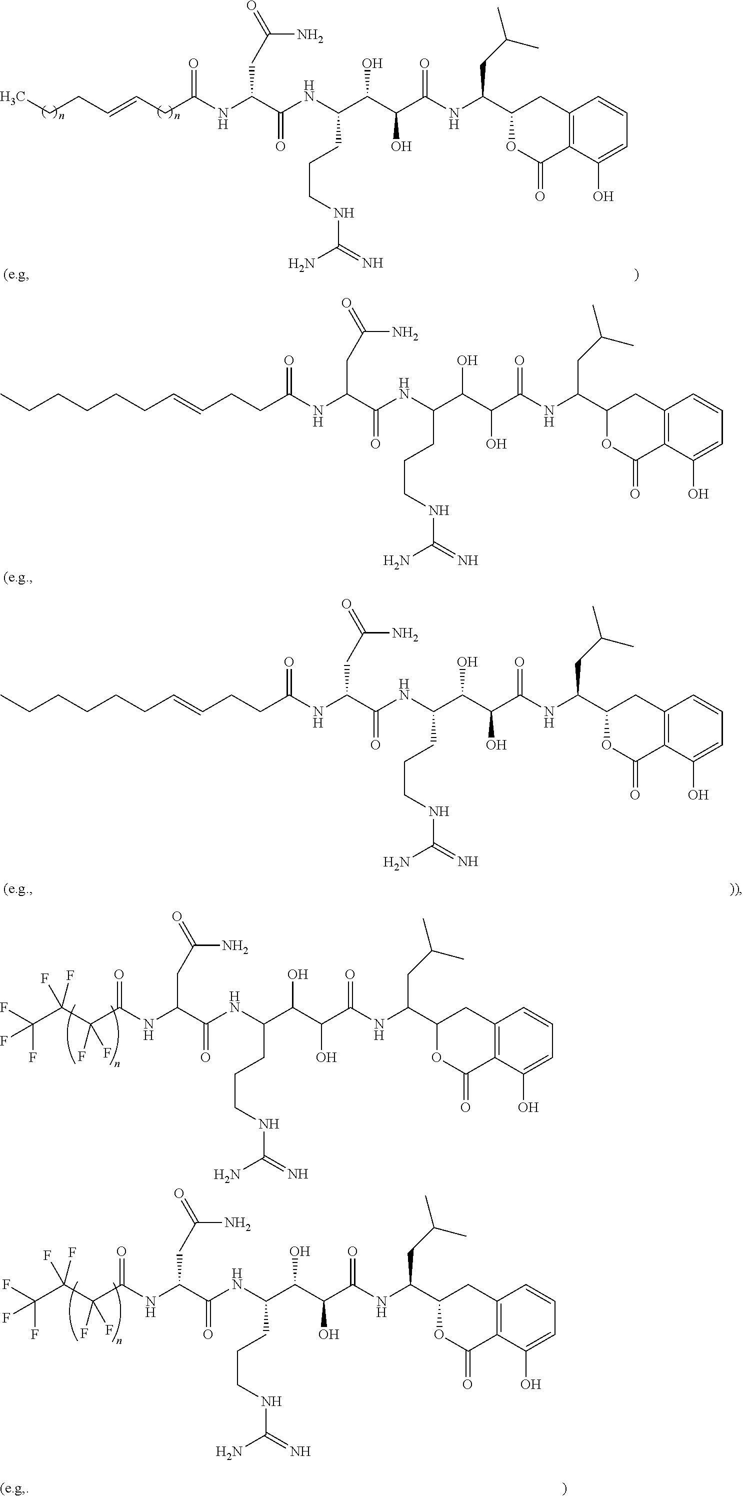

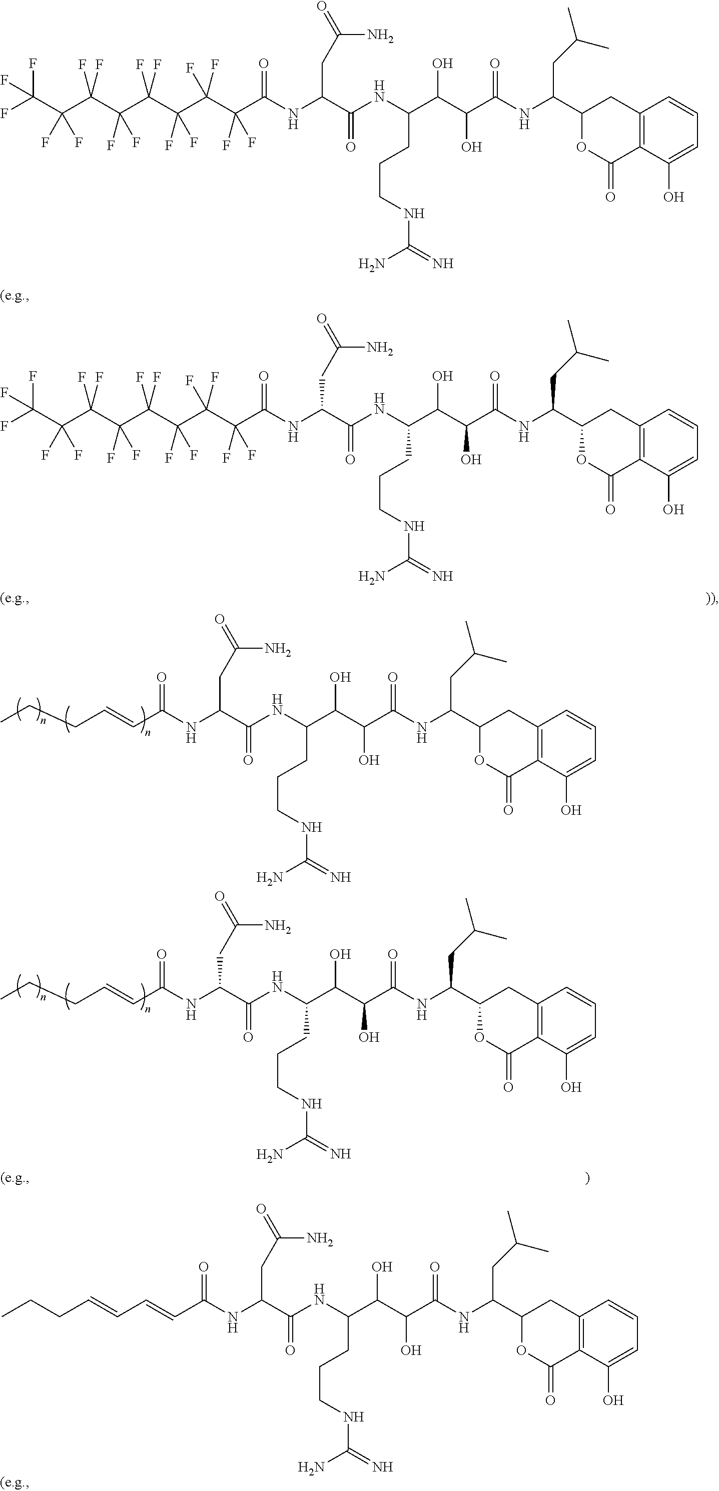

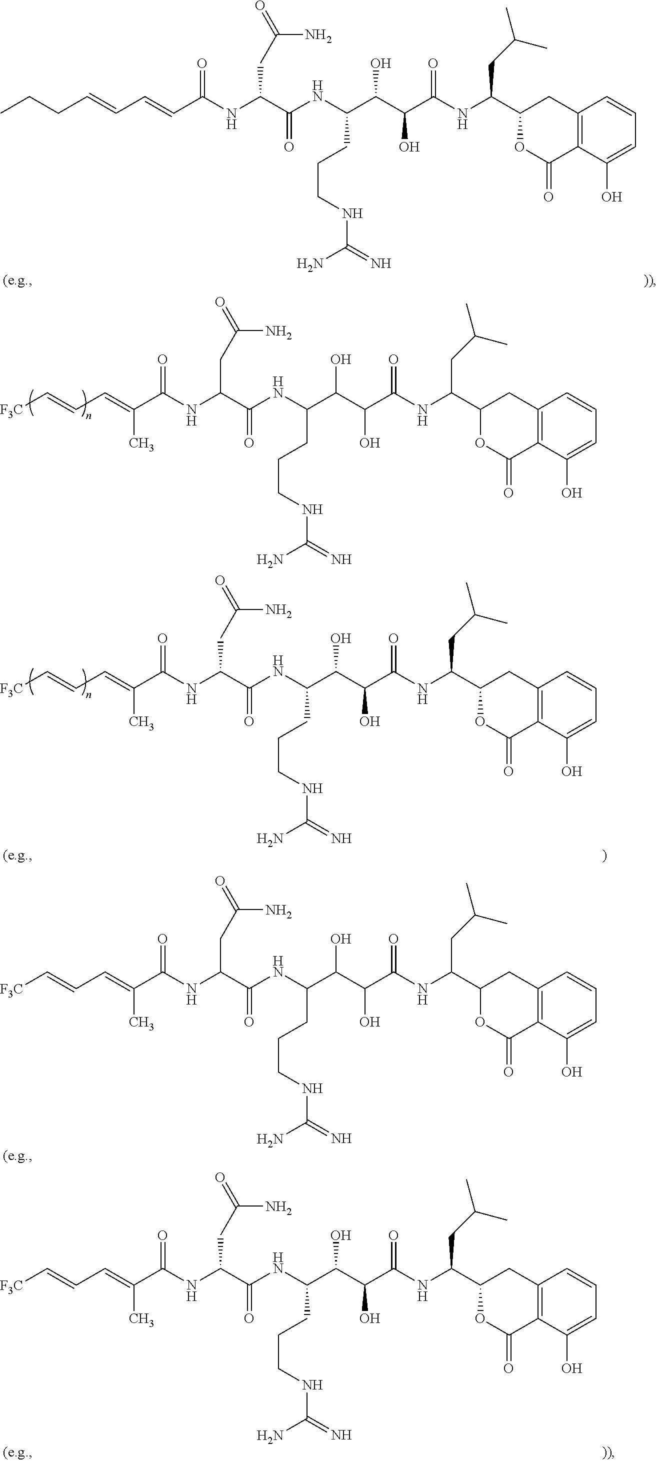

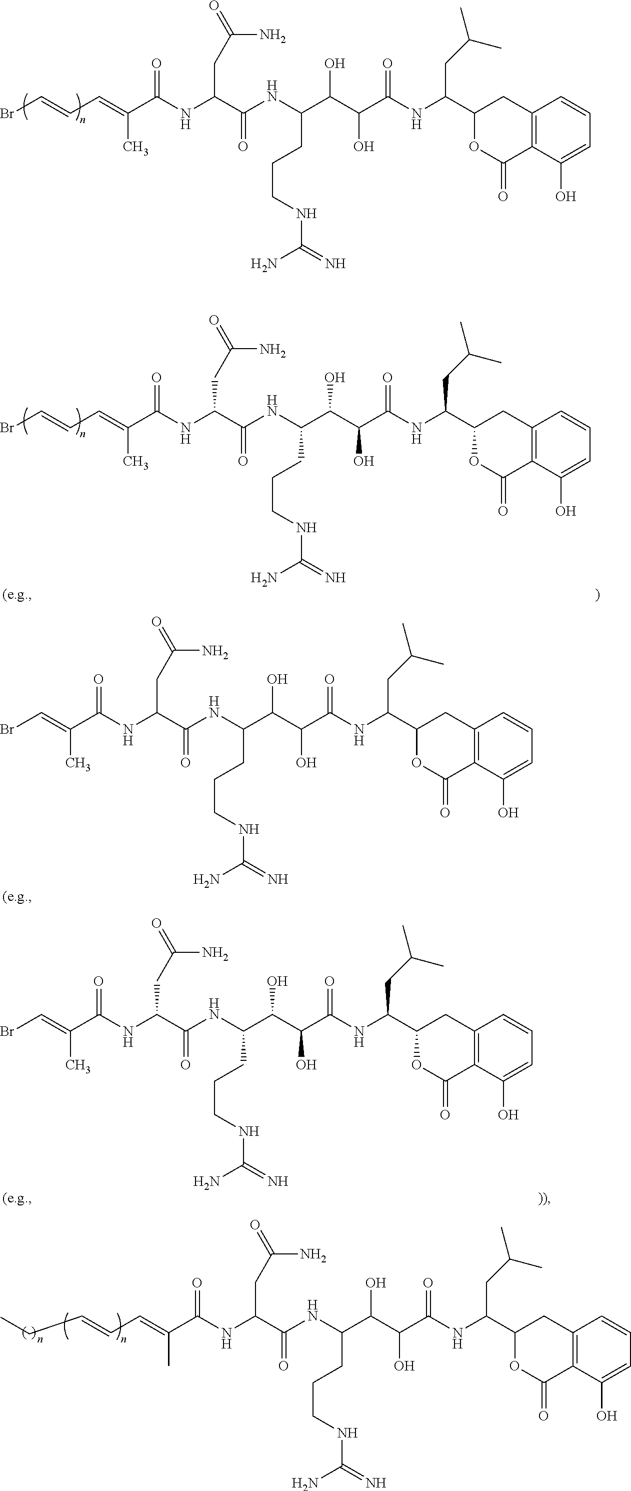

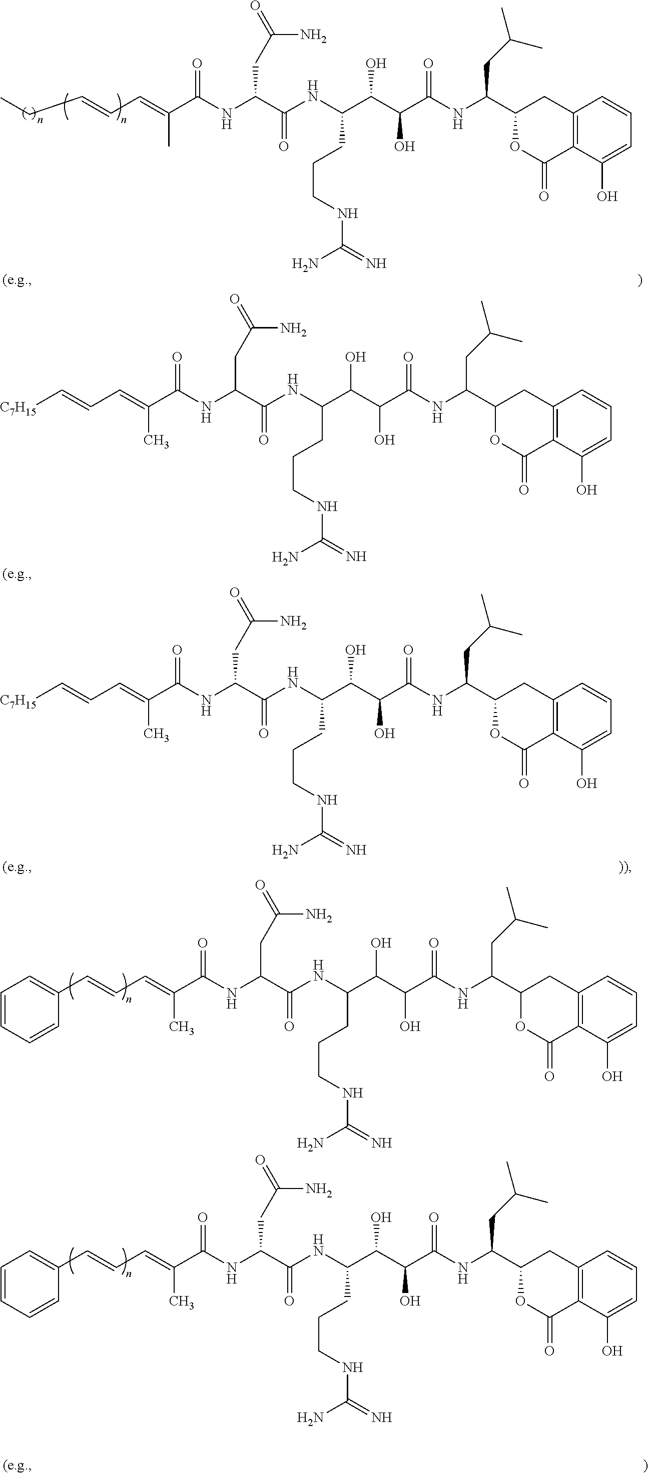

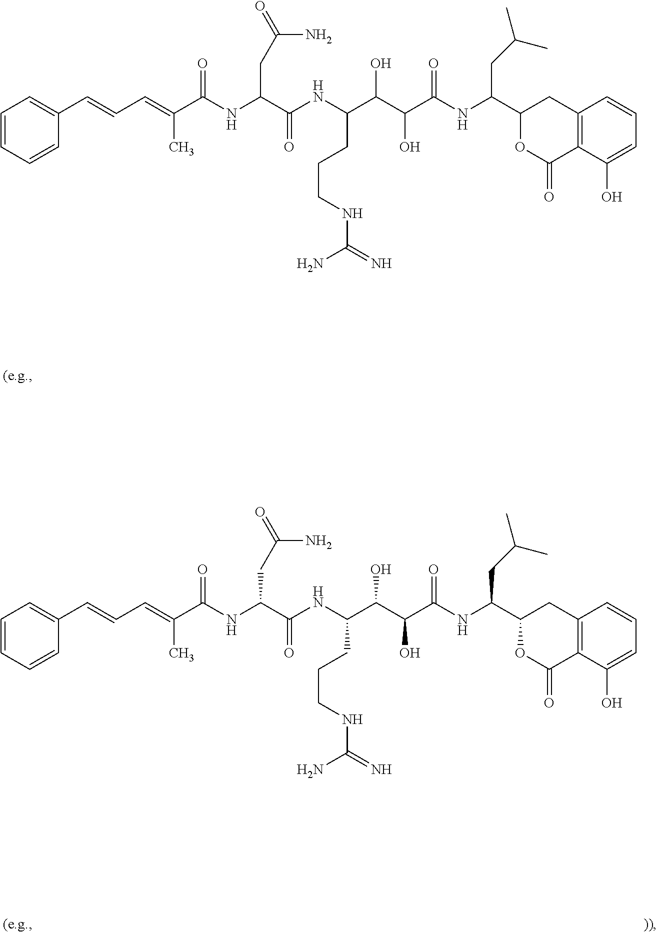 [SMILES: C/C(=C\C=C\c1ccccc1)C(=O)NC(CC(N)=O)C(=O)NC(CCCNC(=N)N)C(O)C(O)C(=O)NC(CC(C)C)C1Cc2cccc(O)c2C(=O)O1 ; C/C(=C\C=C\c1ccccc1)C(=O)N[C@H](CC(N)=O)C(=O)N[C@@H](CCCNC(=N)N)[C@H](O)[C@H](O)C(=O)N[C@@H](CC(C)C)[C@@H]1Cc2cccc(O)c2C(=O)O1]

or any combination thereof, wherein each n is independently 0 to 18.
Statement 14. A method according to any one of Statements 2 to 5, wherein the prodrug antibiotic has the following structure:

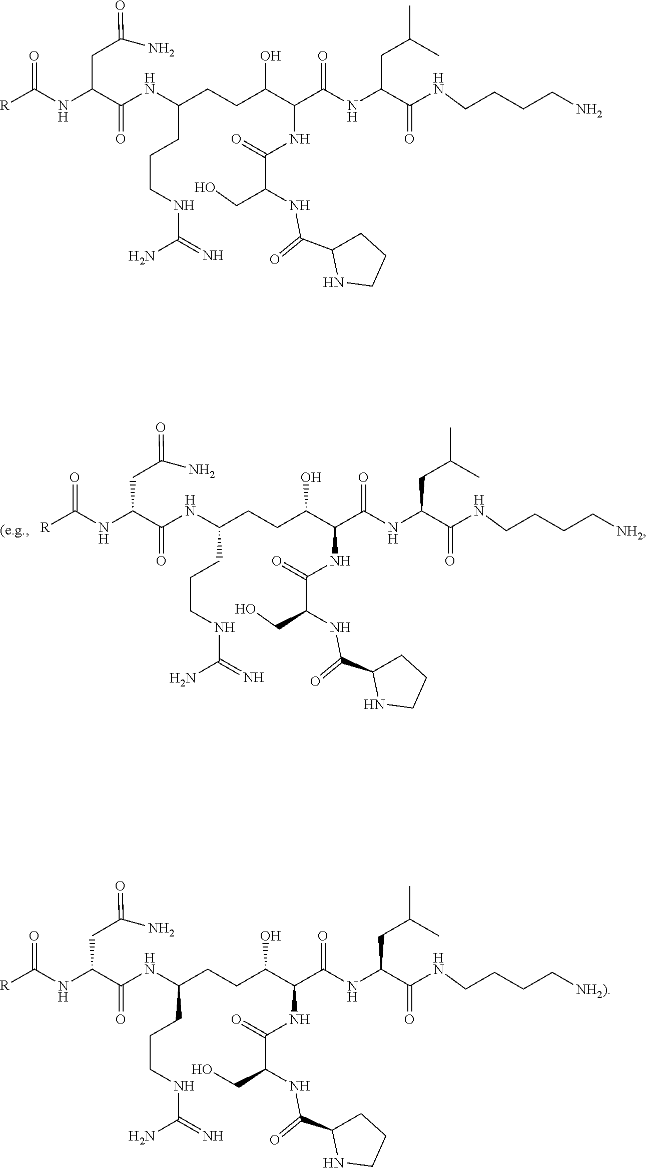

Statement 15. A method according to any one of Statements 2 to 5, wherein the prodrug antibiotic has the following structure:

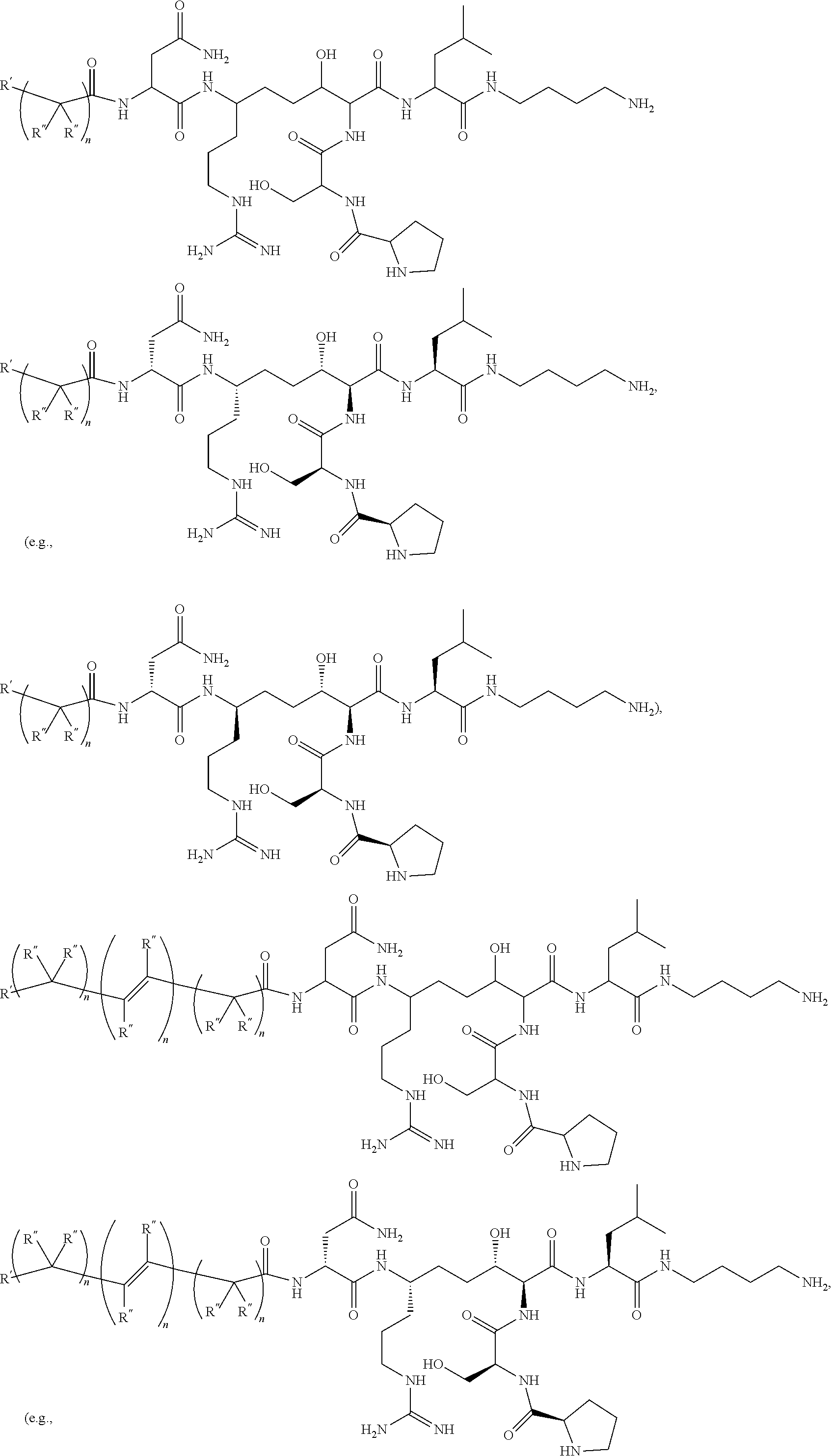

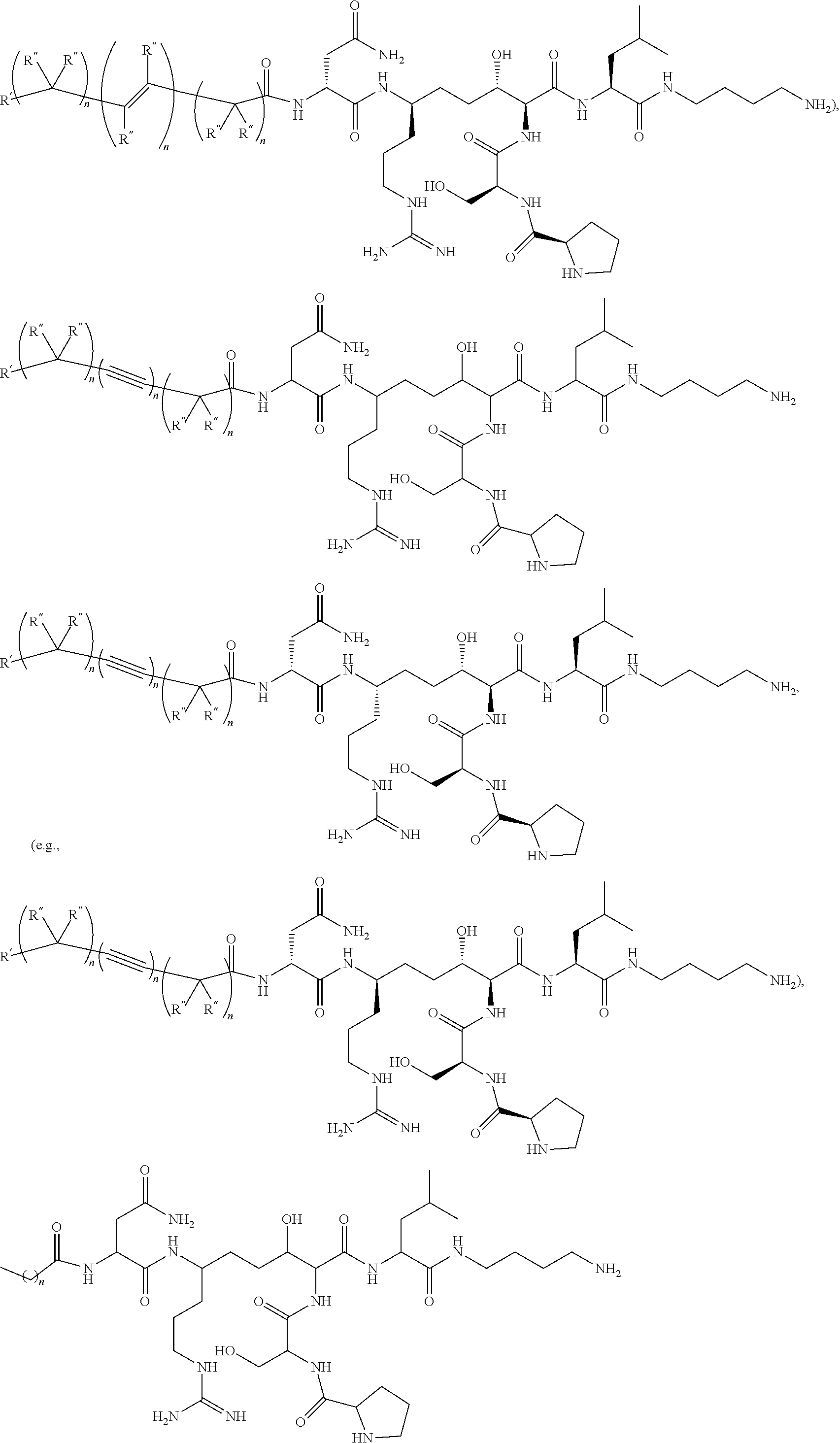

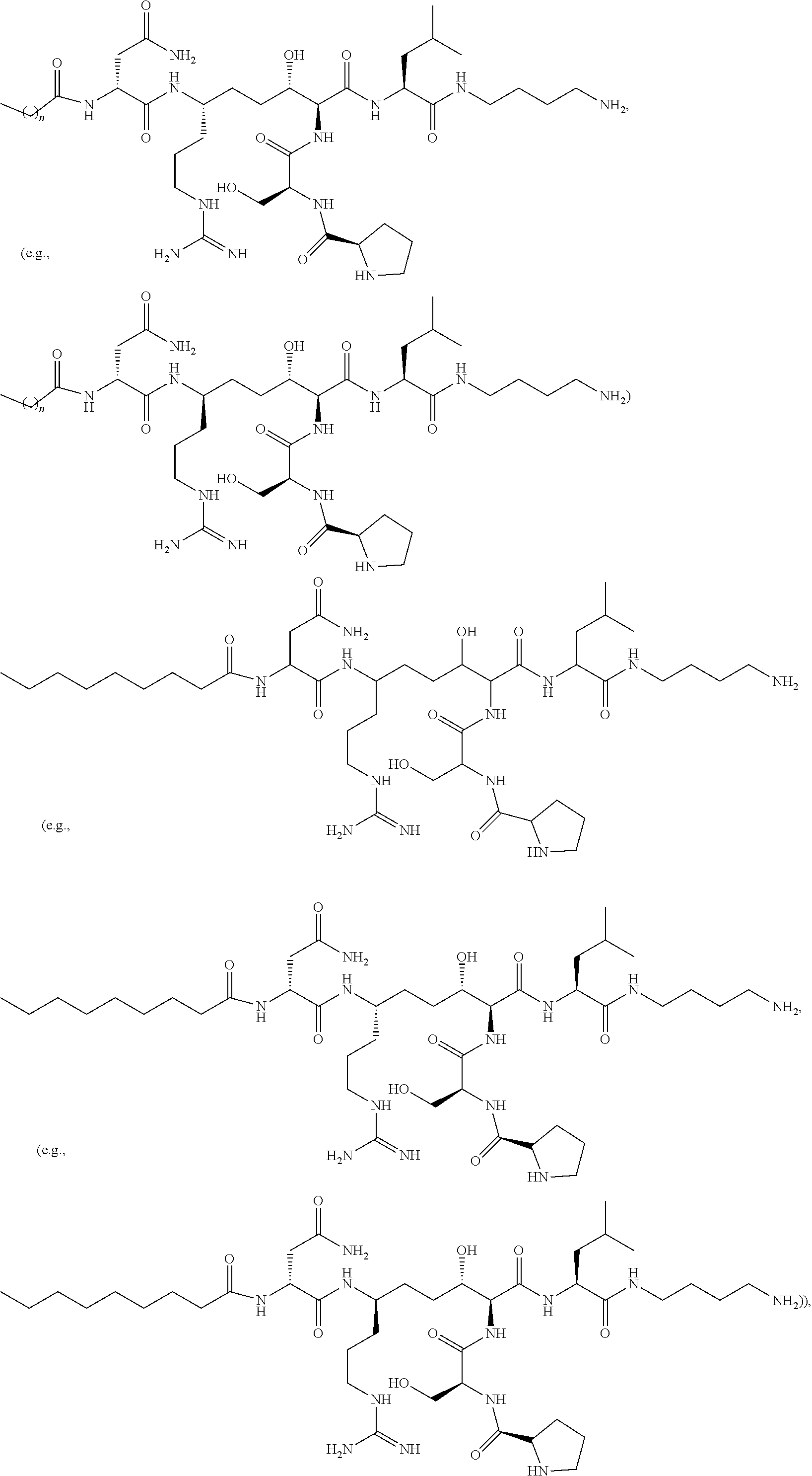

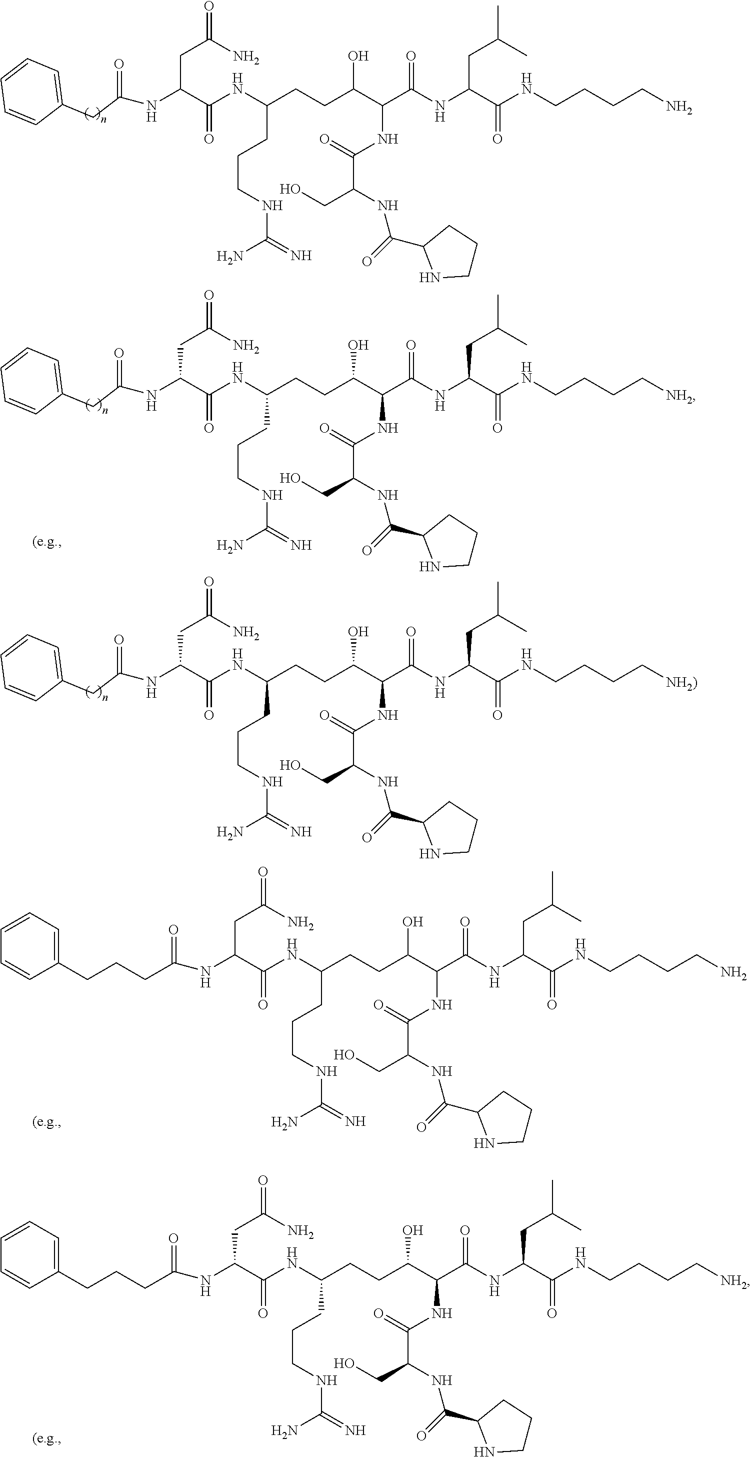

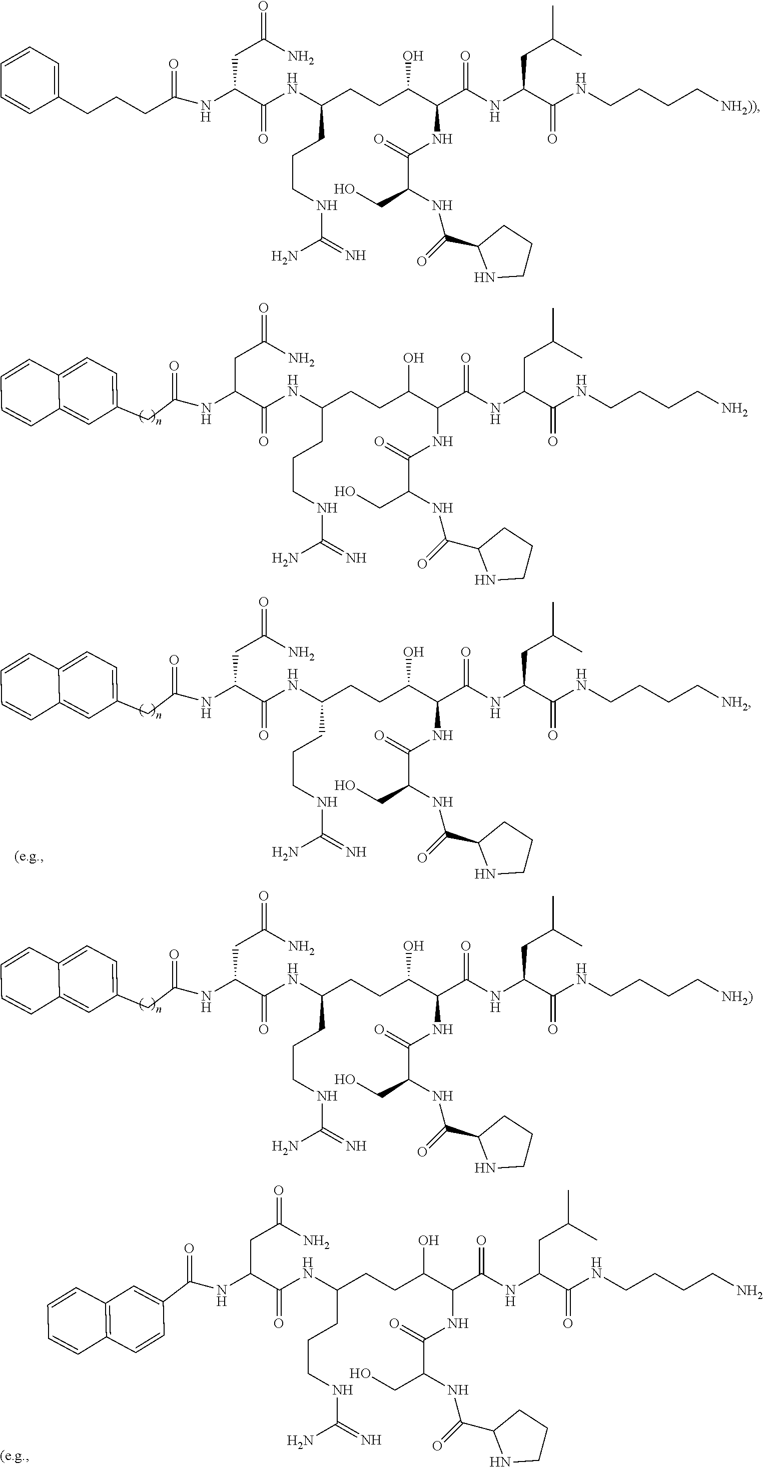

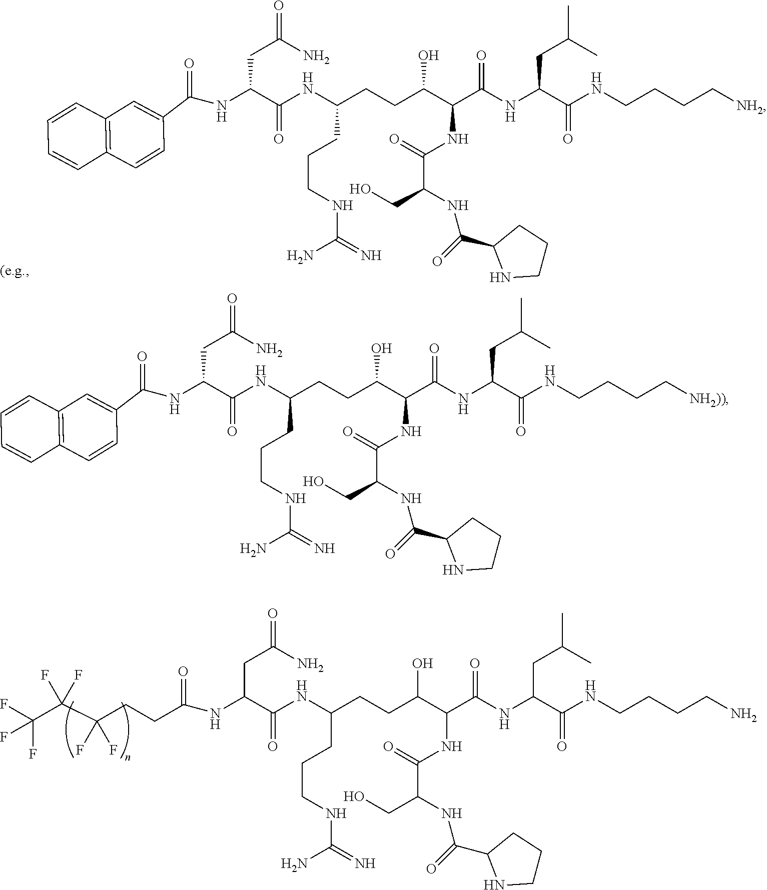

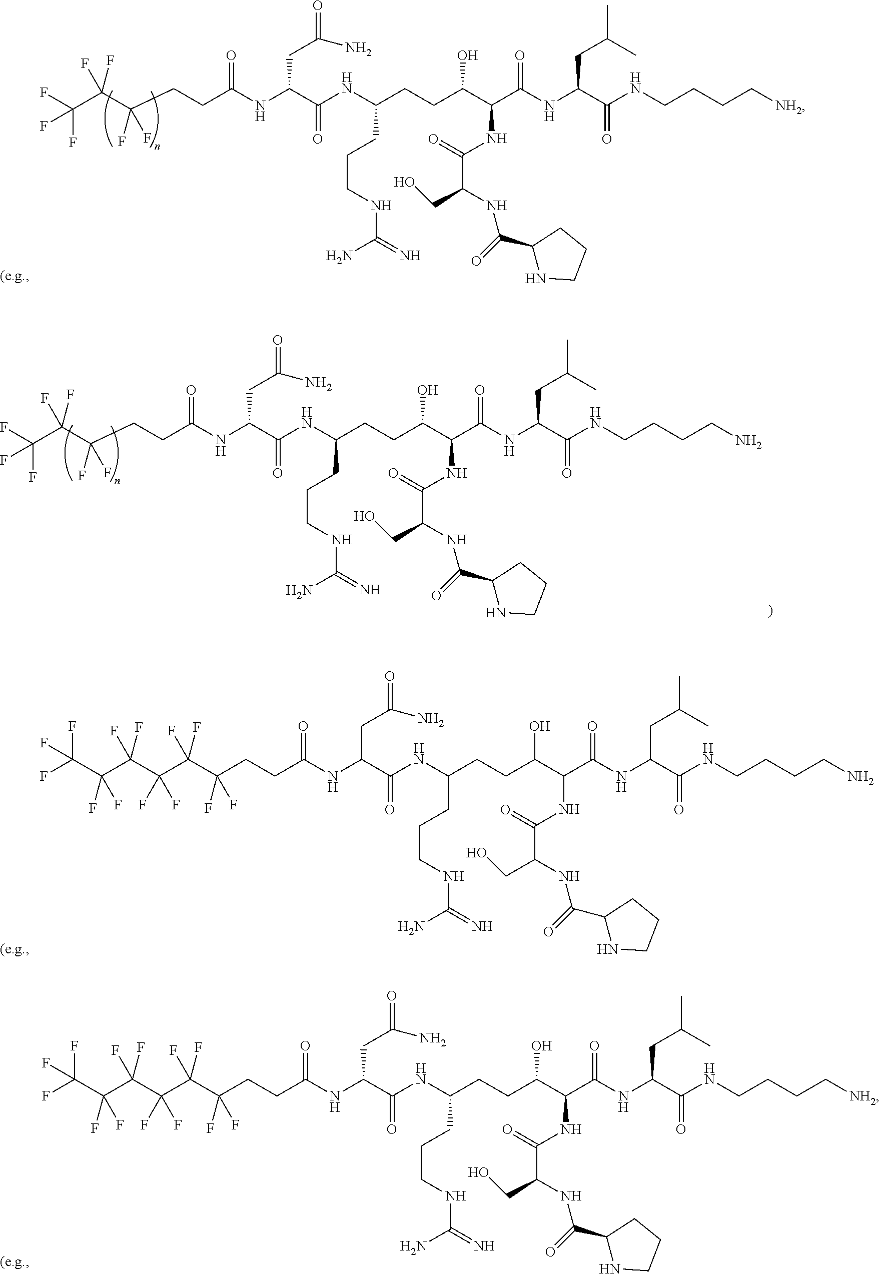

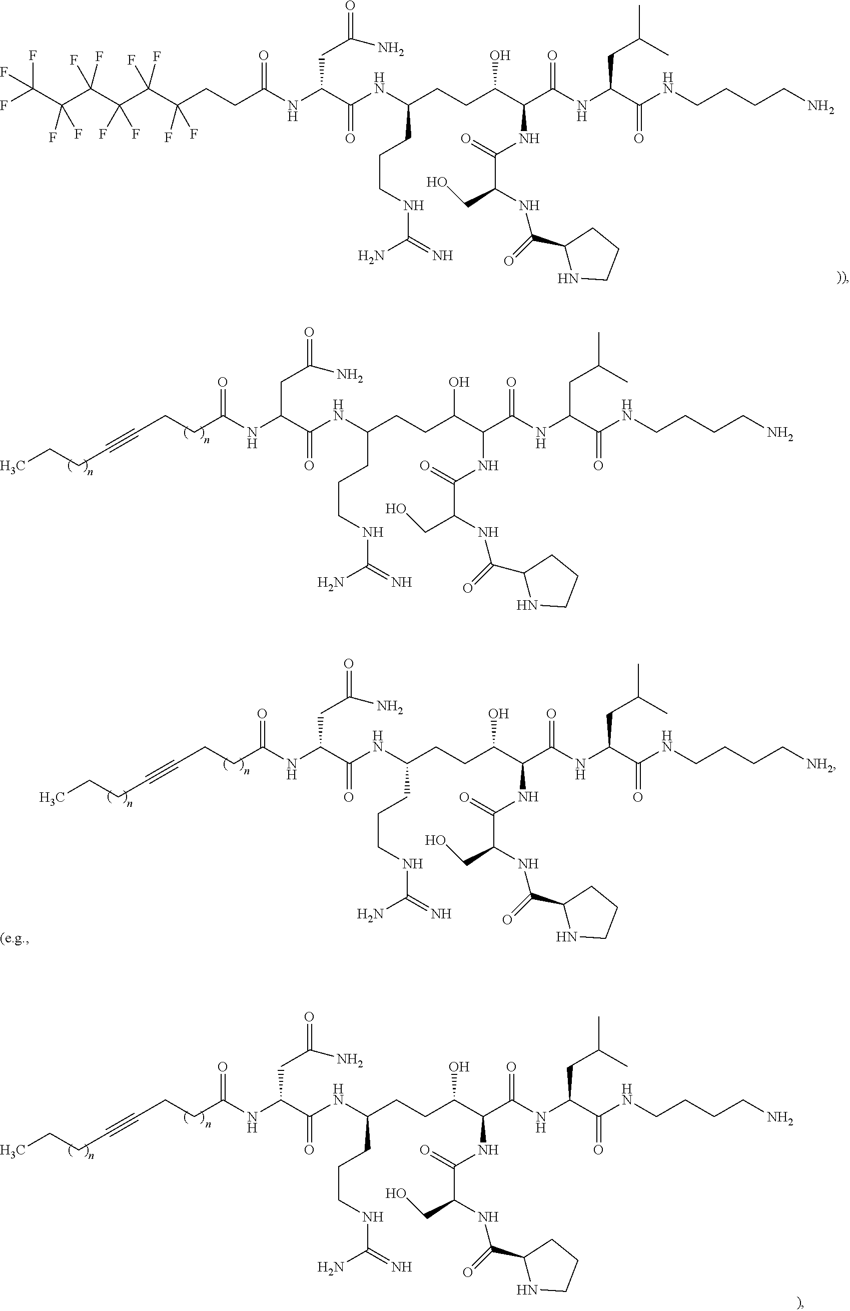

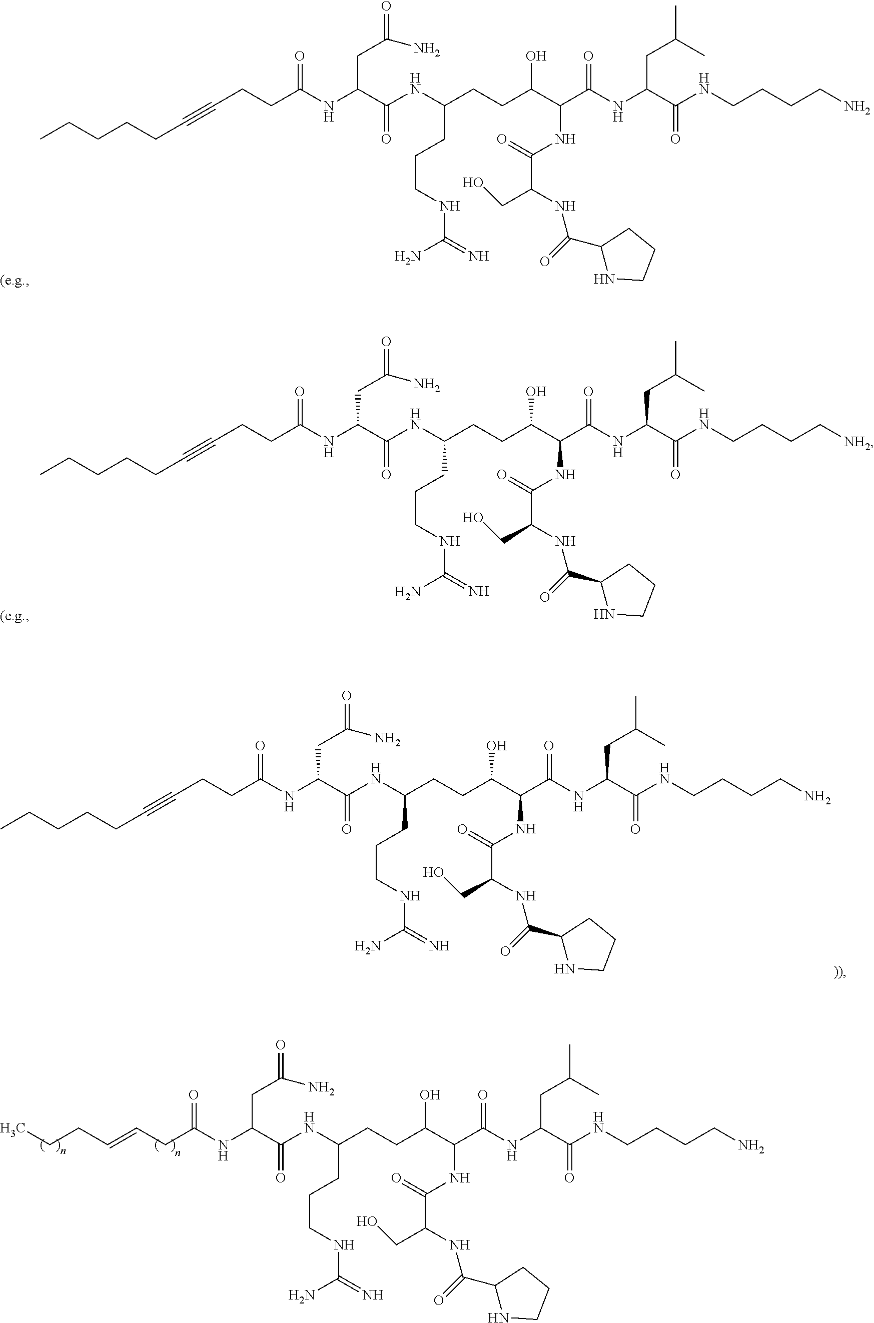

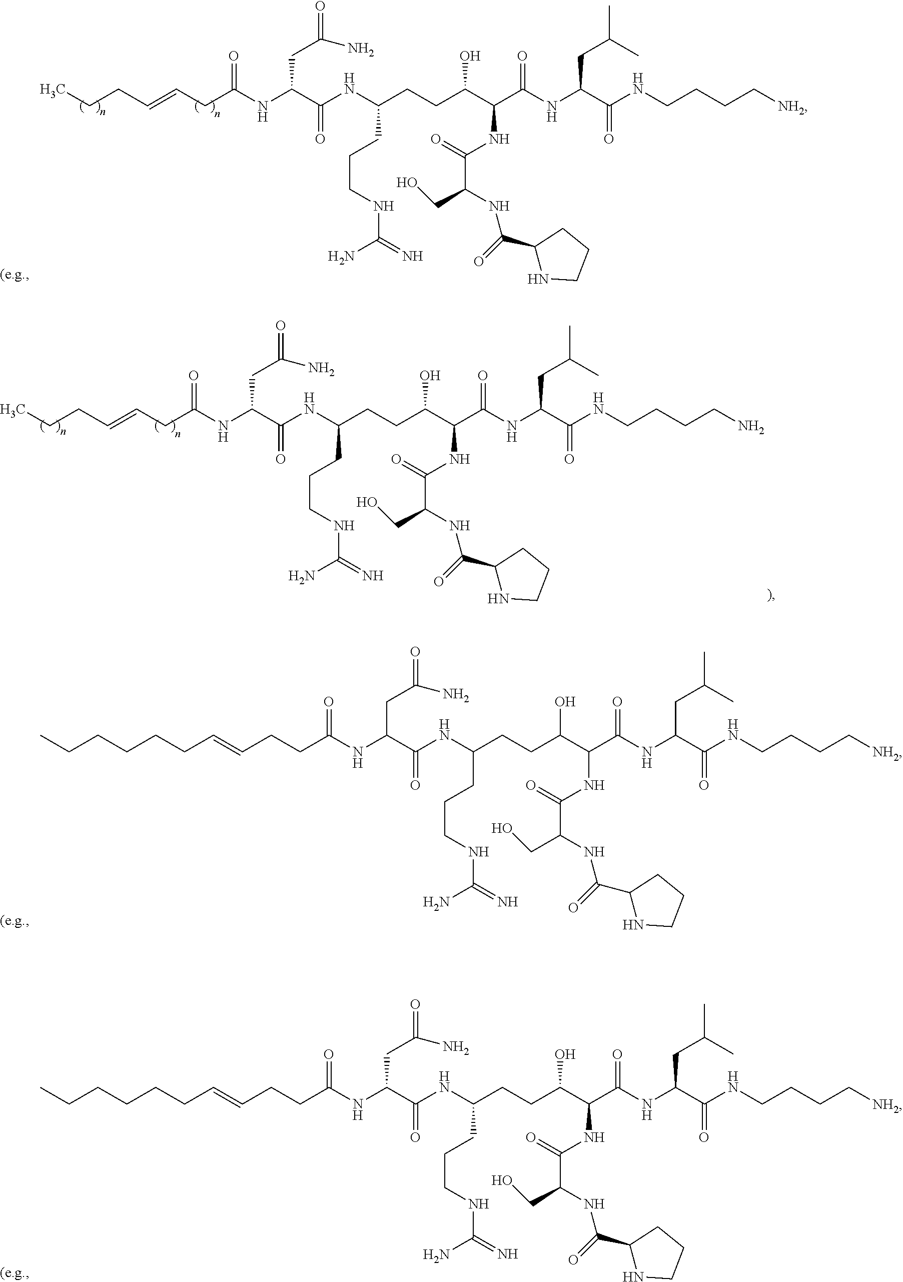

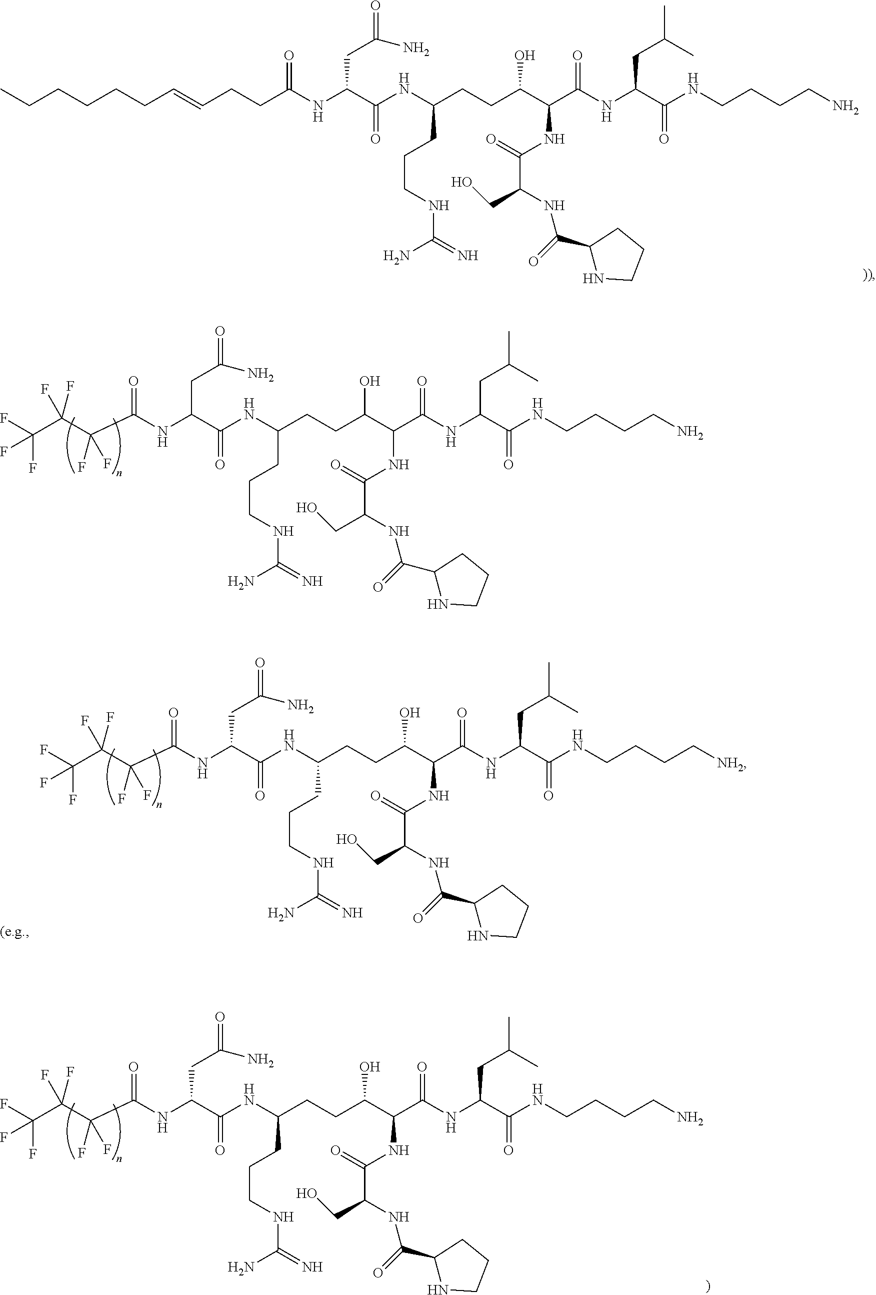

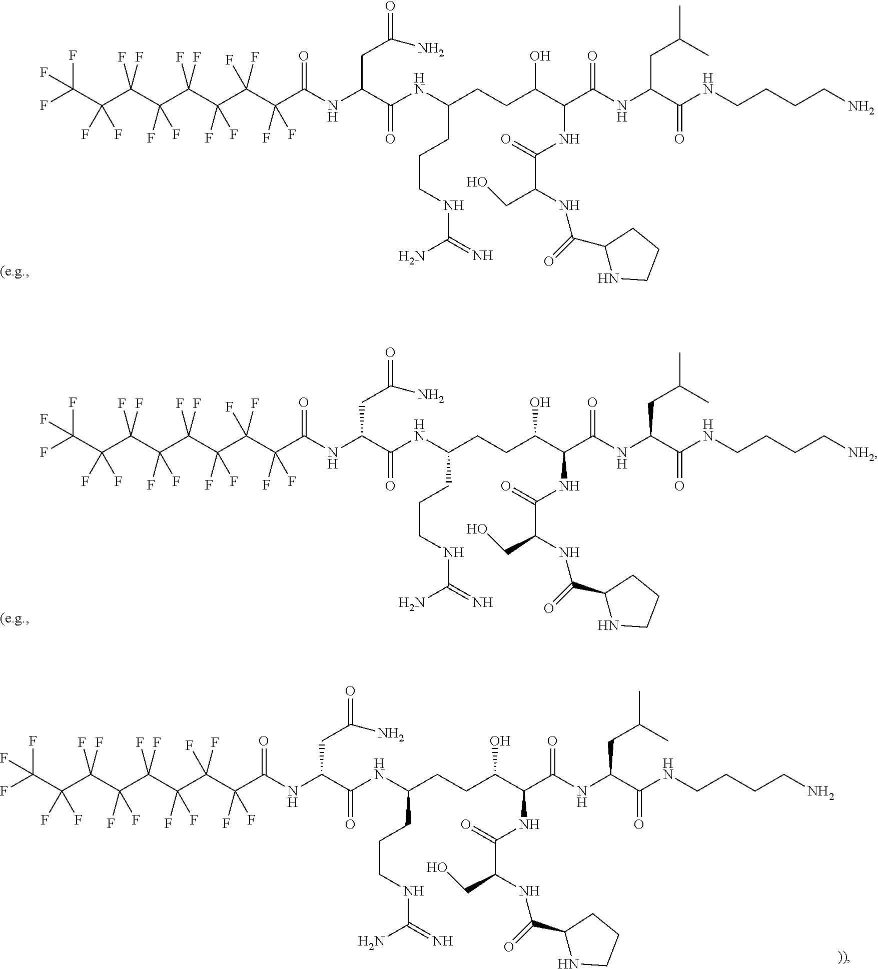

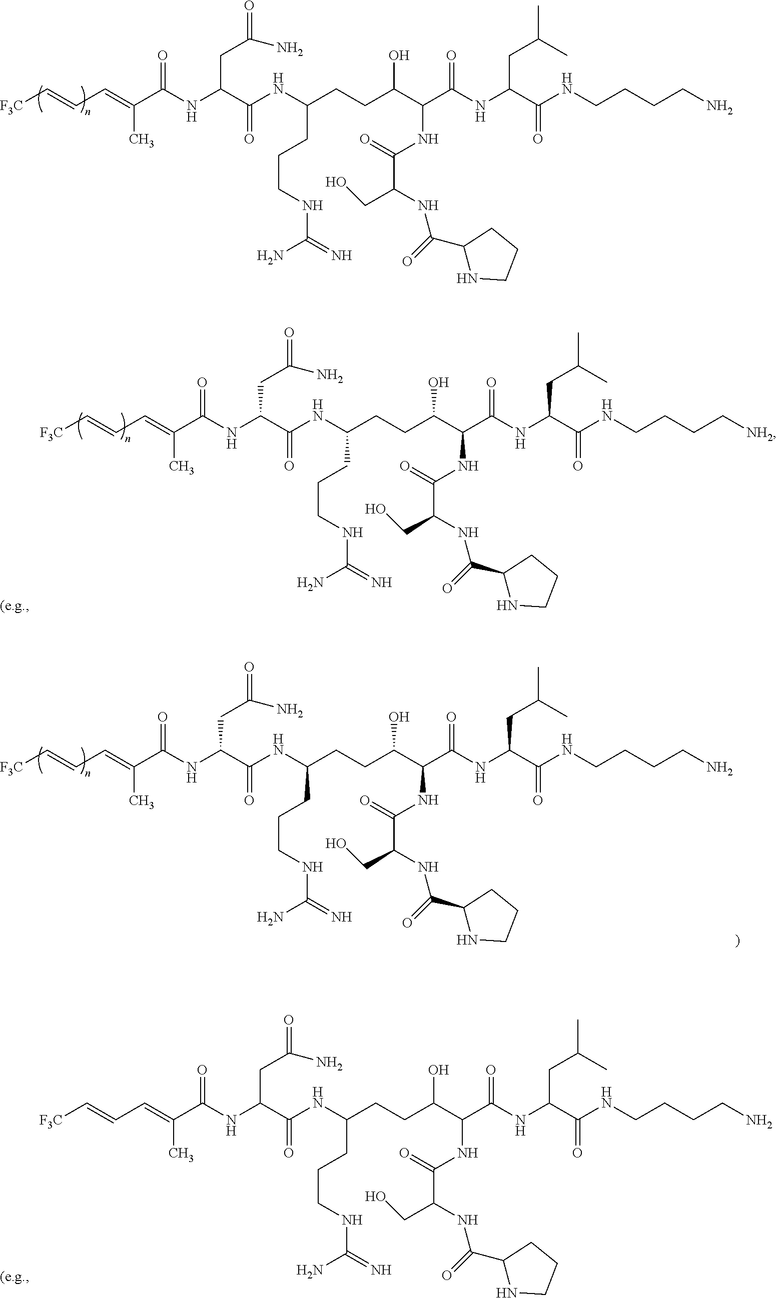

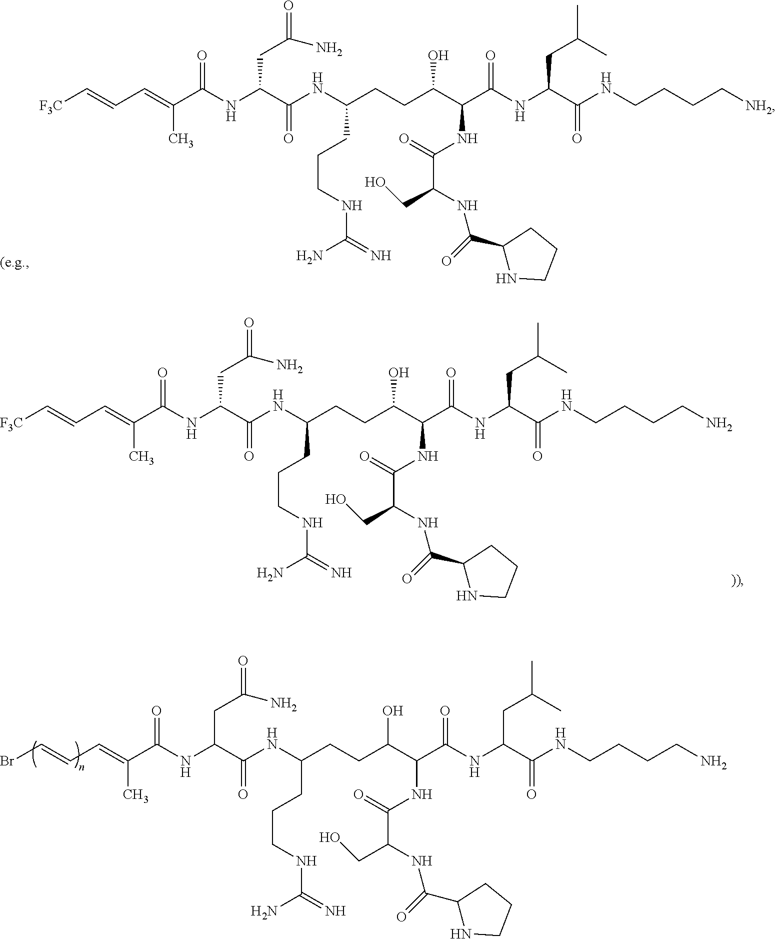

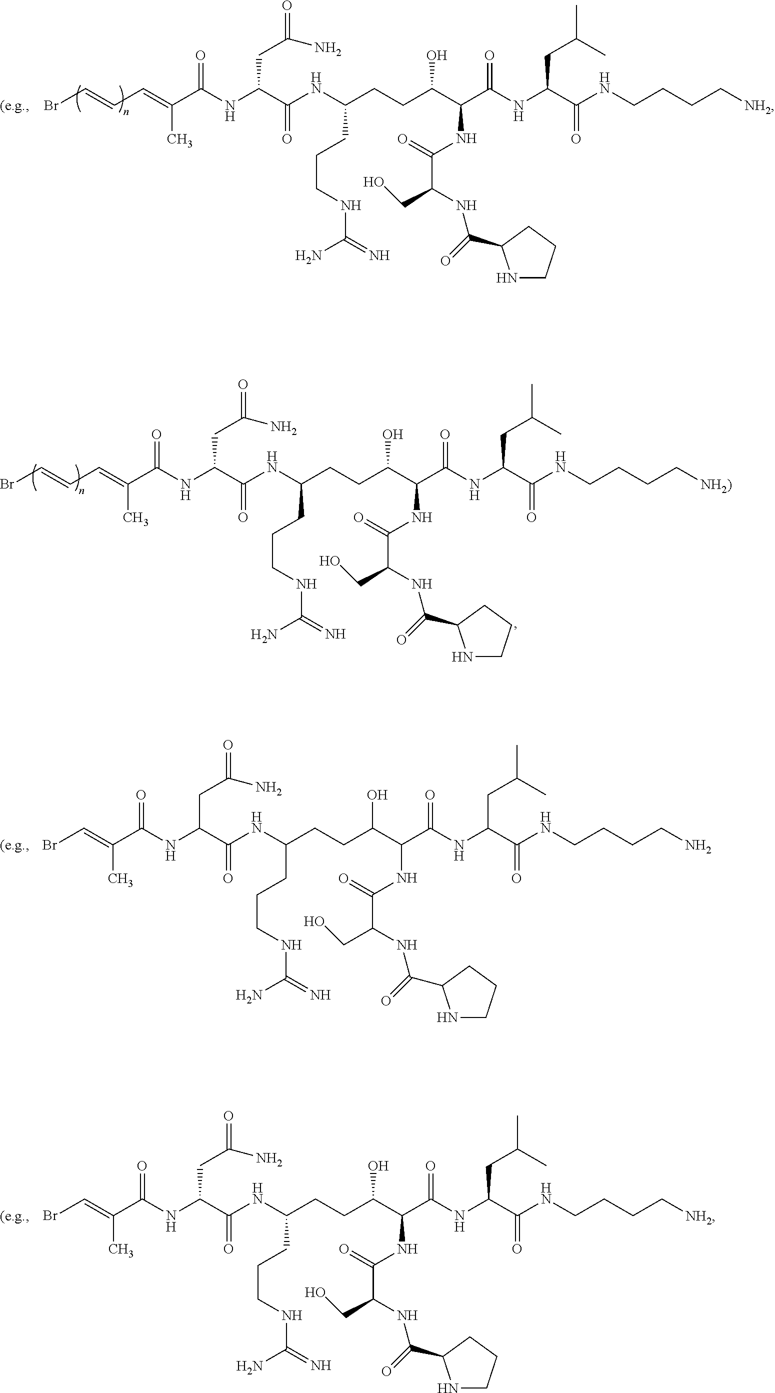

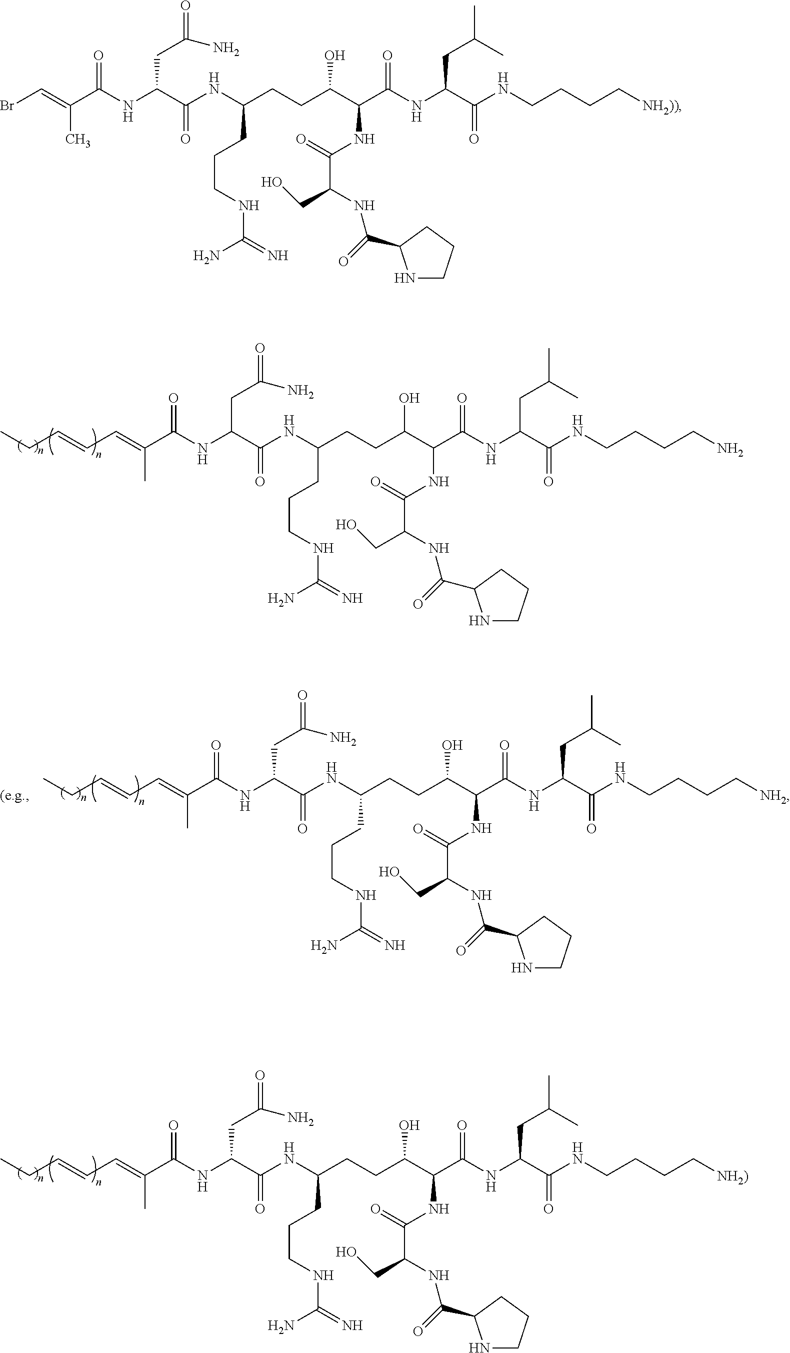

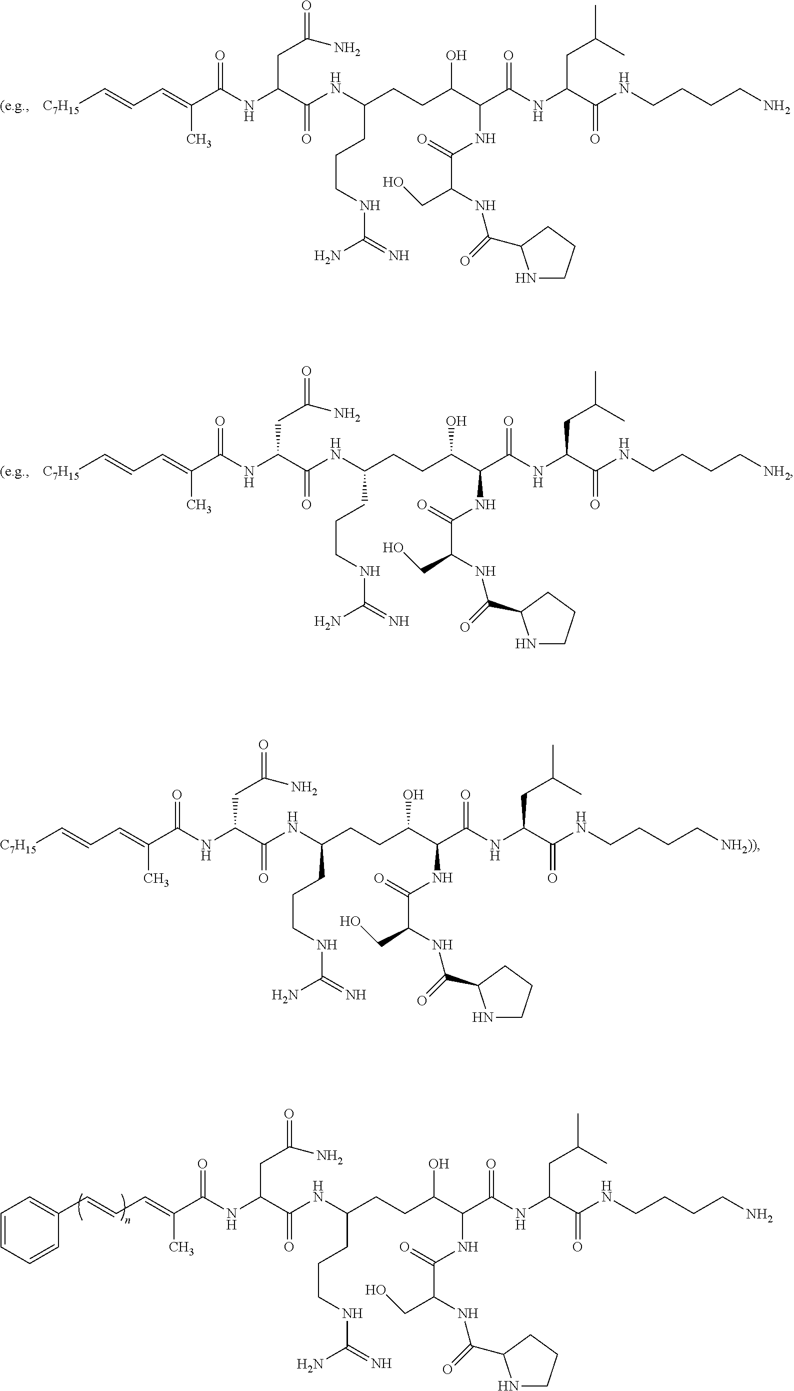

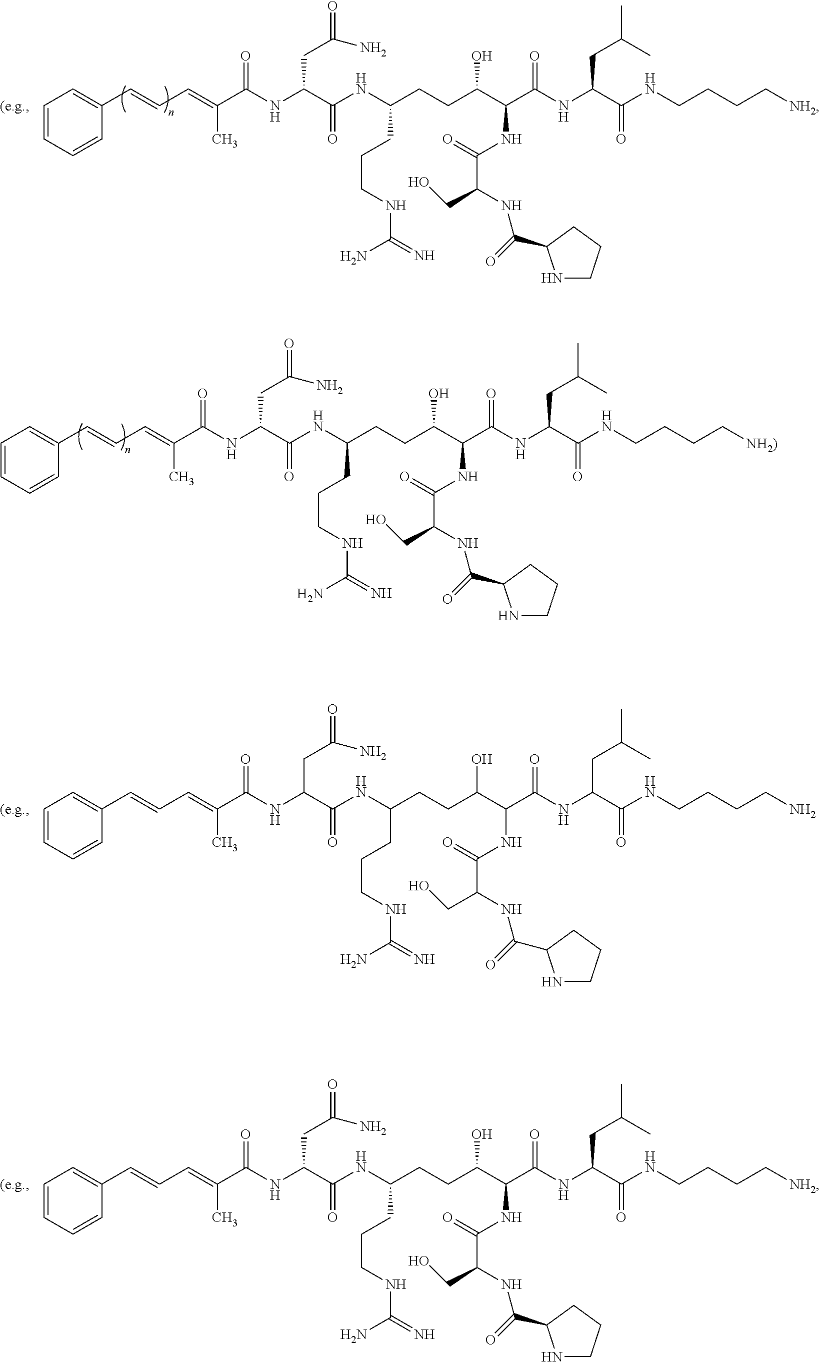

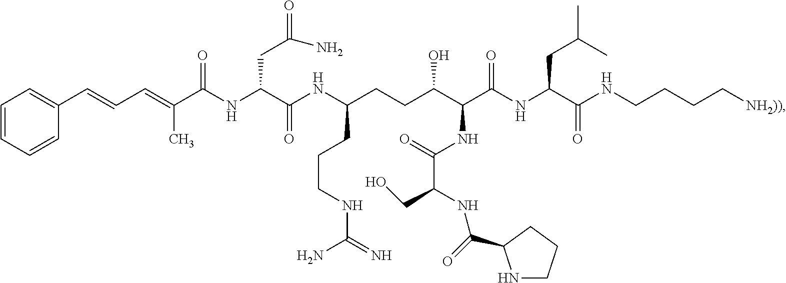

or any combination thereof, wherein n is 0 to 18.
Statement 16. A method according to any one of Statements 2 to 5, wherein the prodrug antibiotic has the following structure:

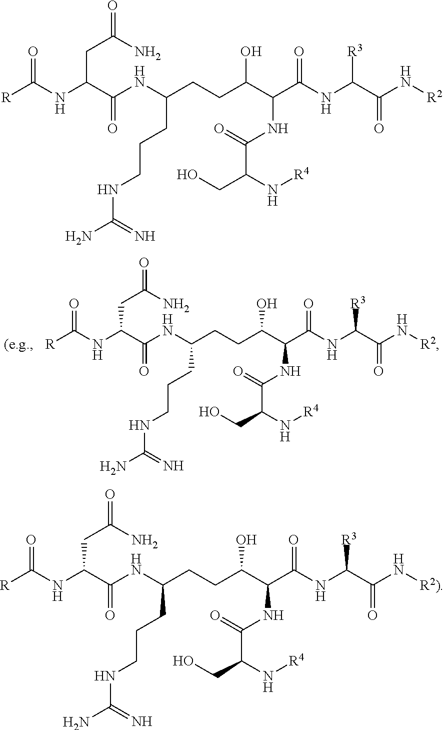

Statement 17. A method according to claim 10a, wherein the prodrug antibiotic has the following structure:

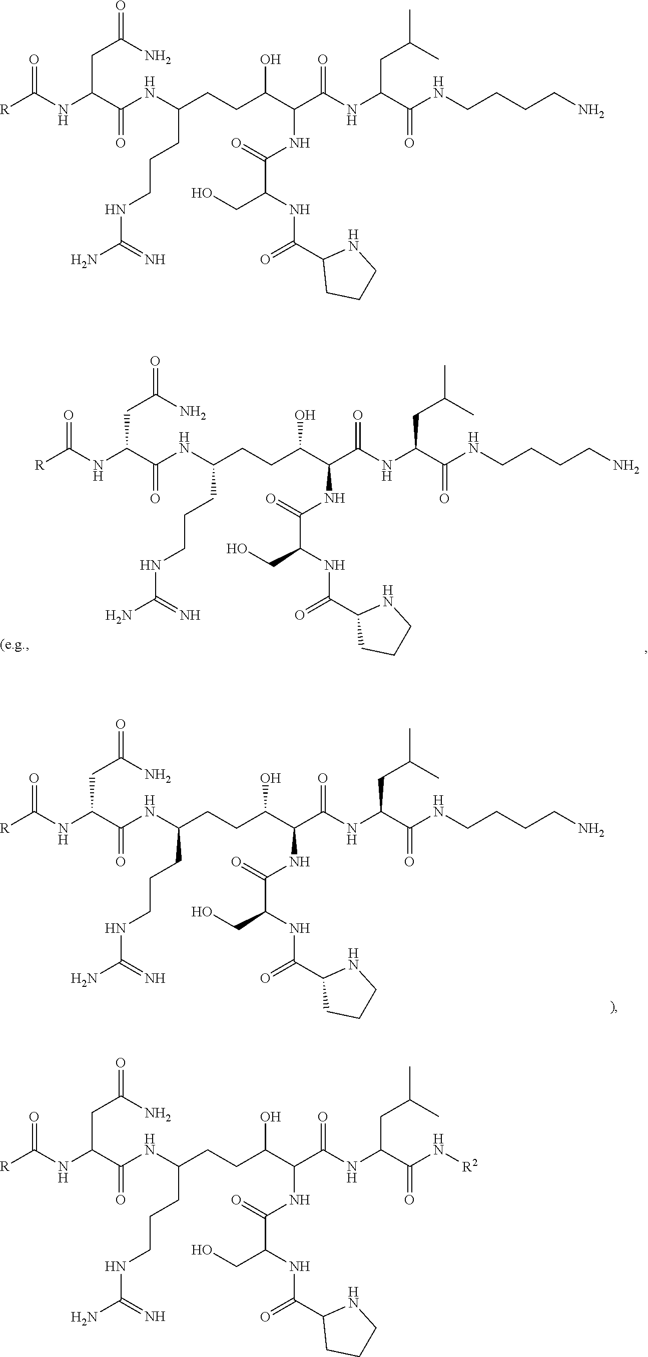

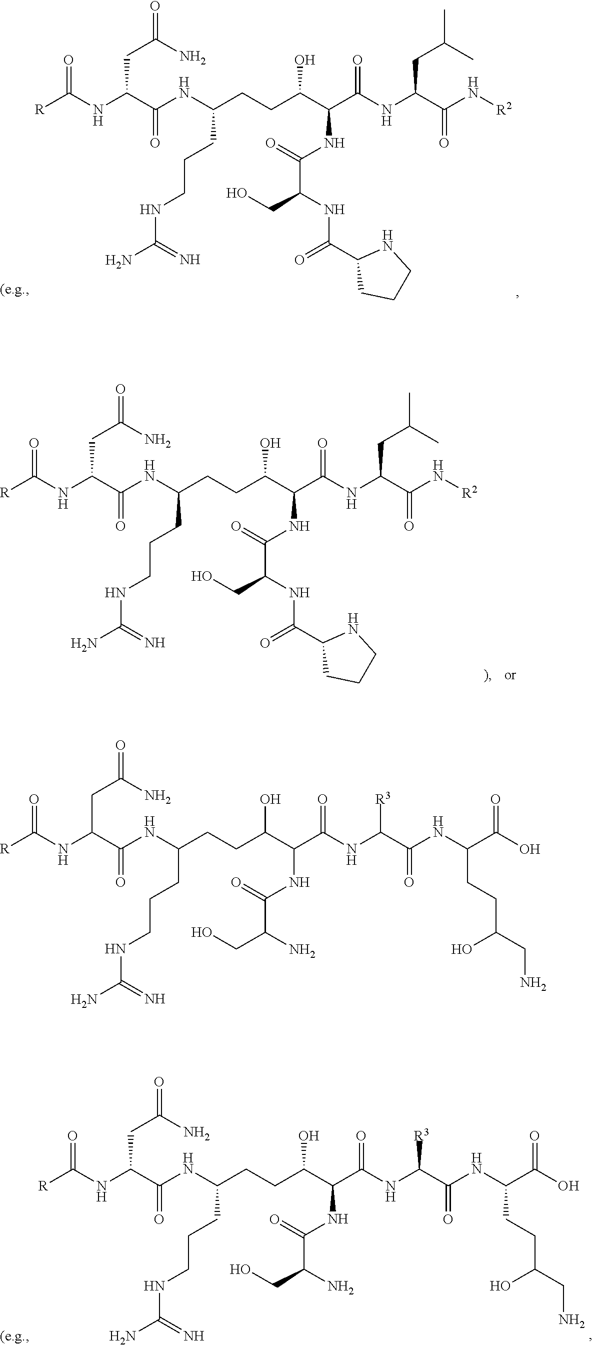

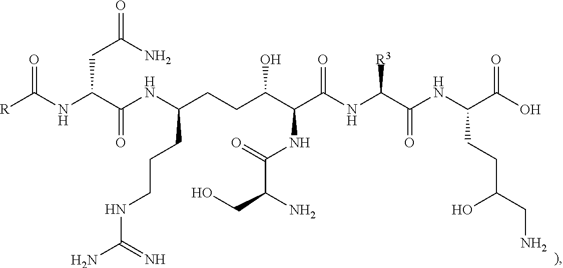

Statement 18. A method according to Statement 17, wherein the prodrug antibiotic has the following structure:

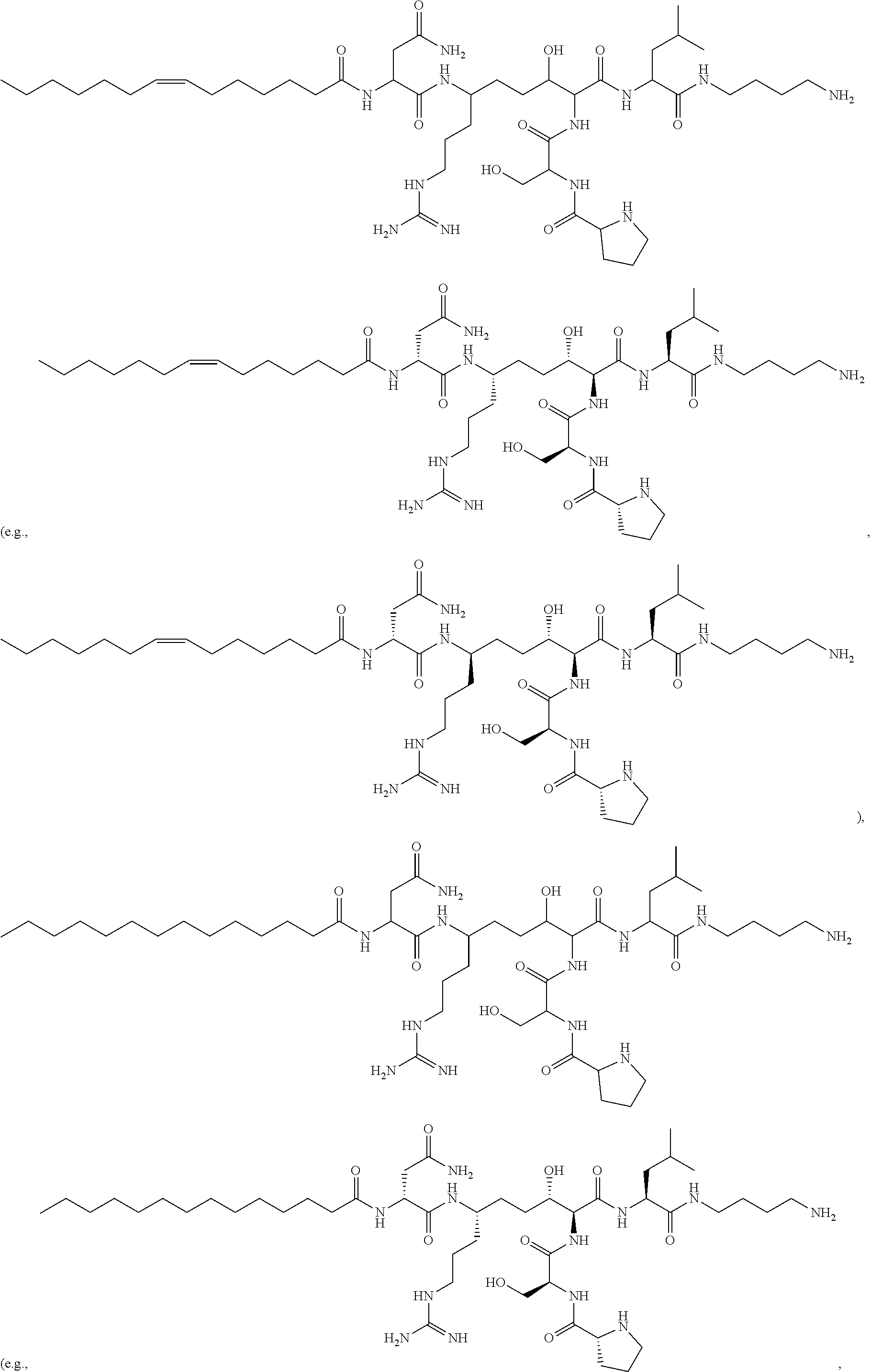

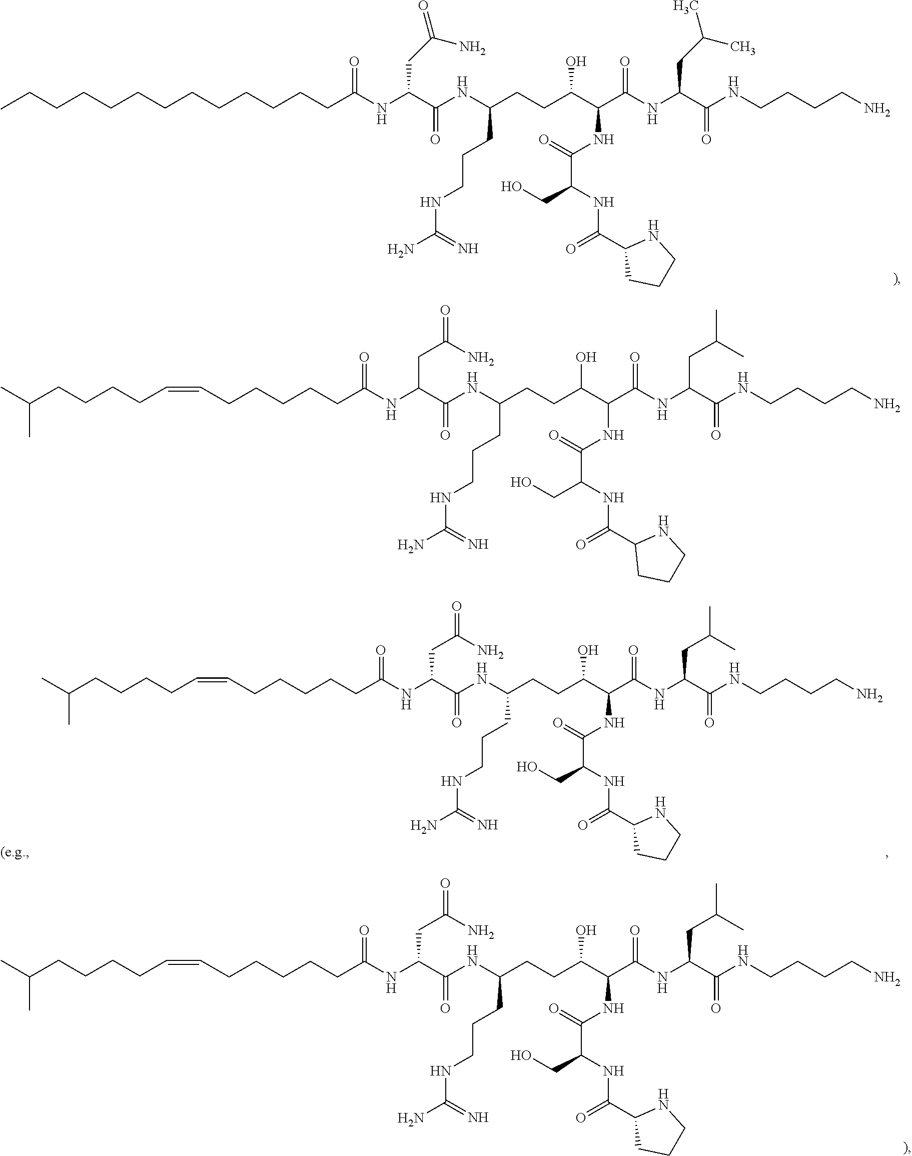

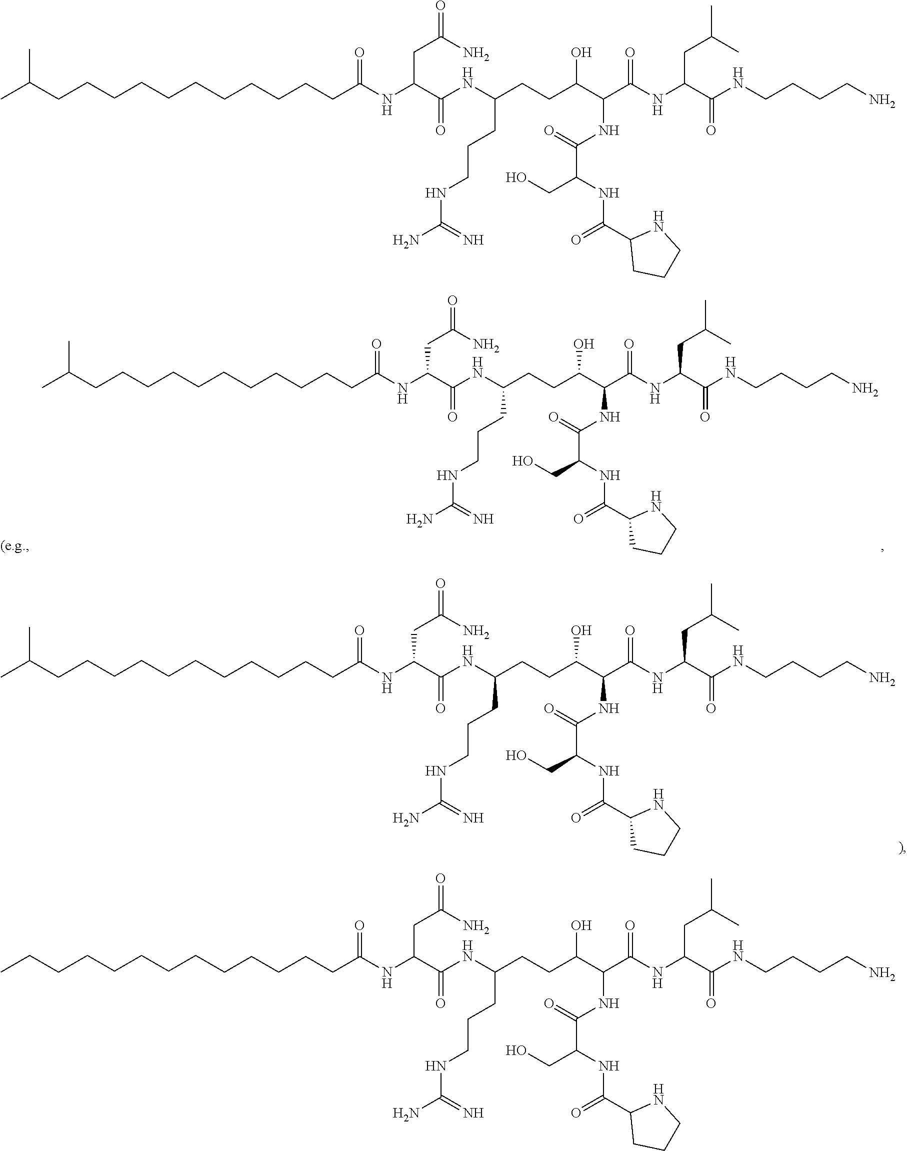

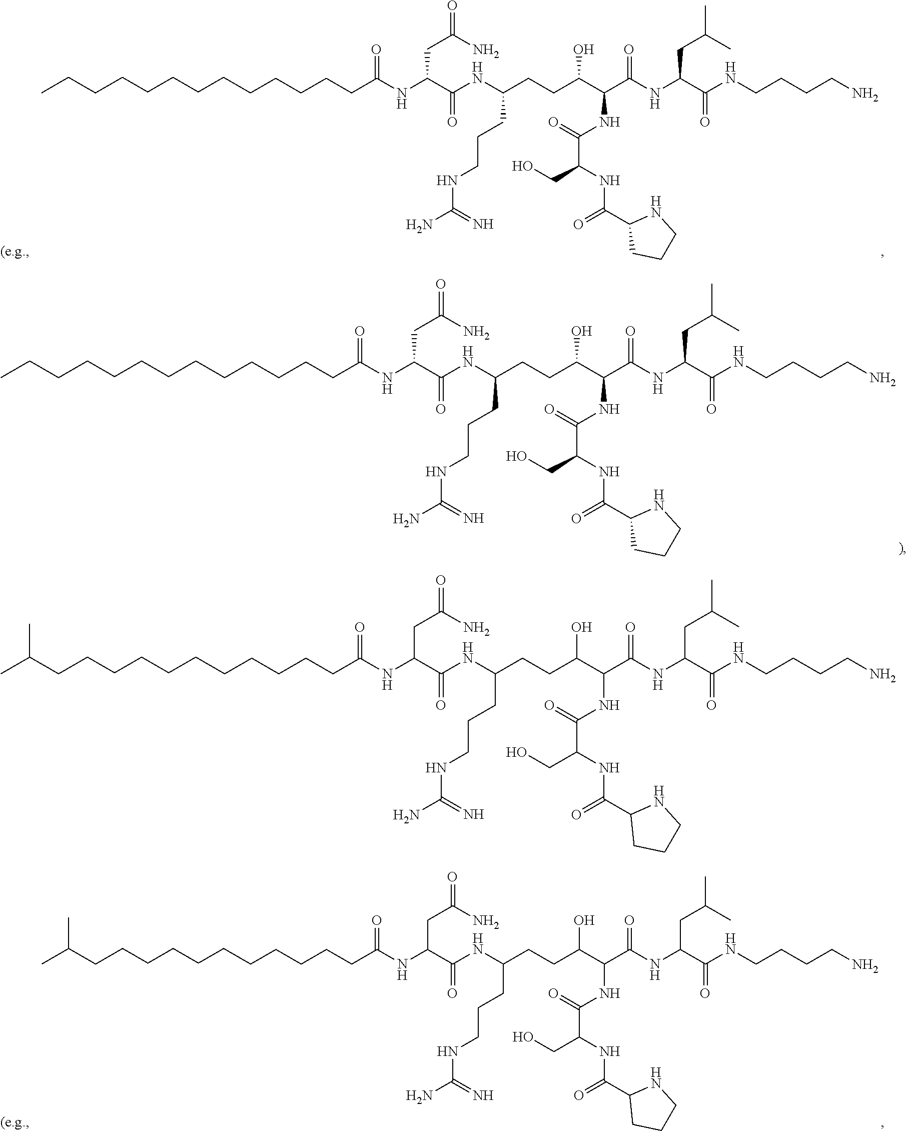

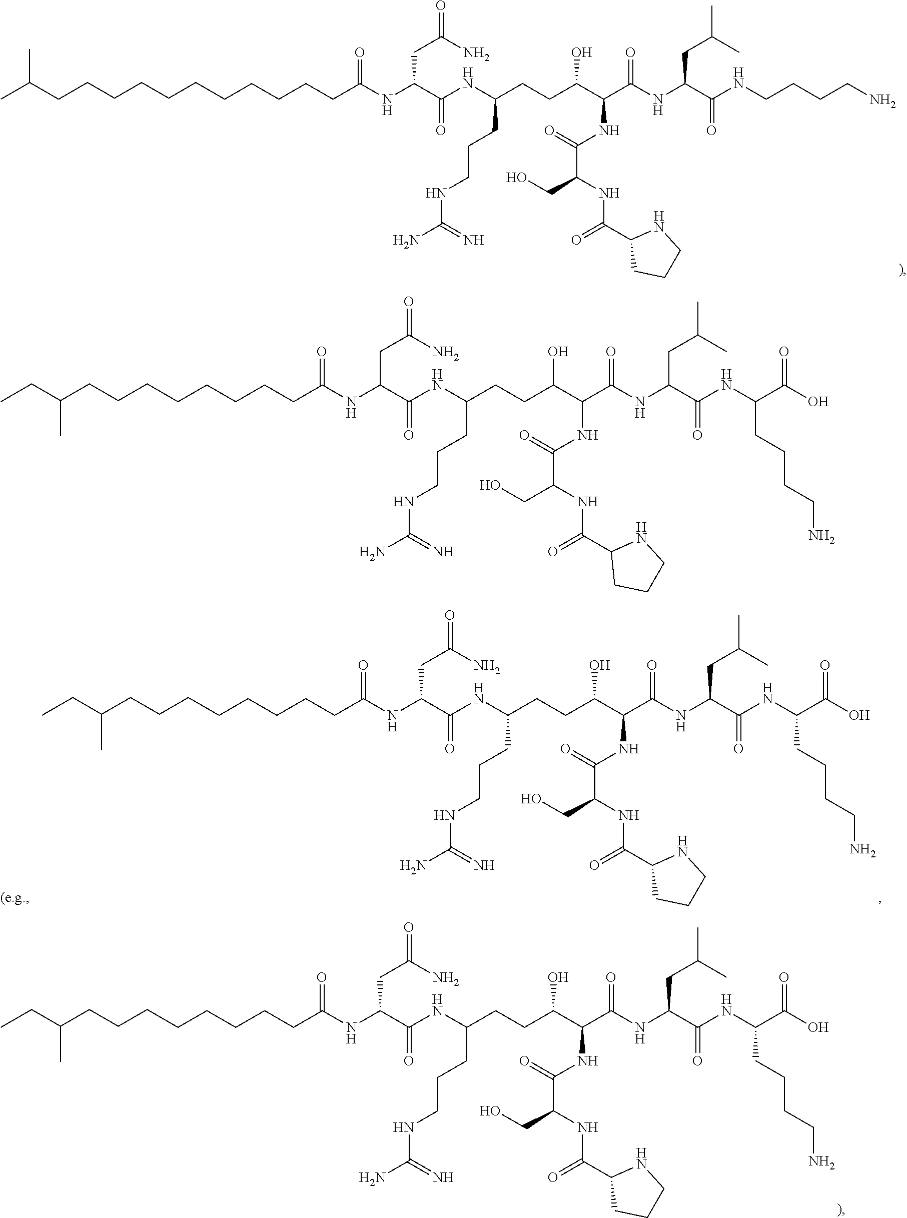

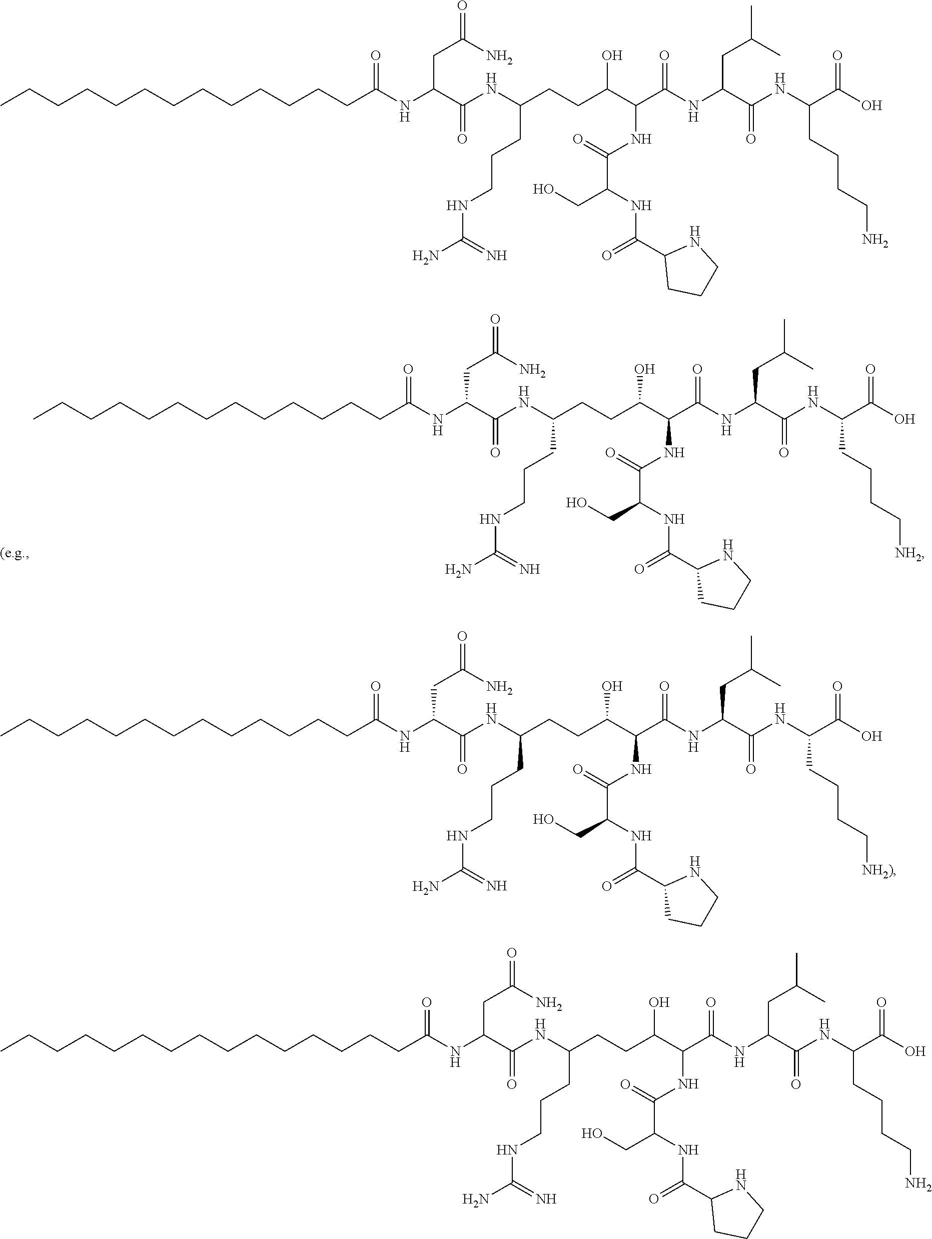

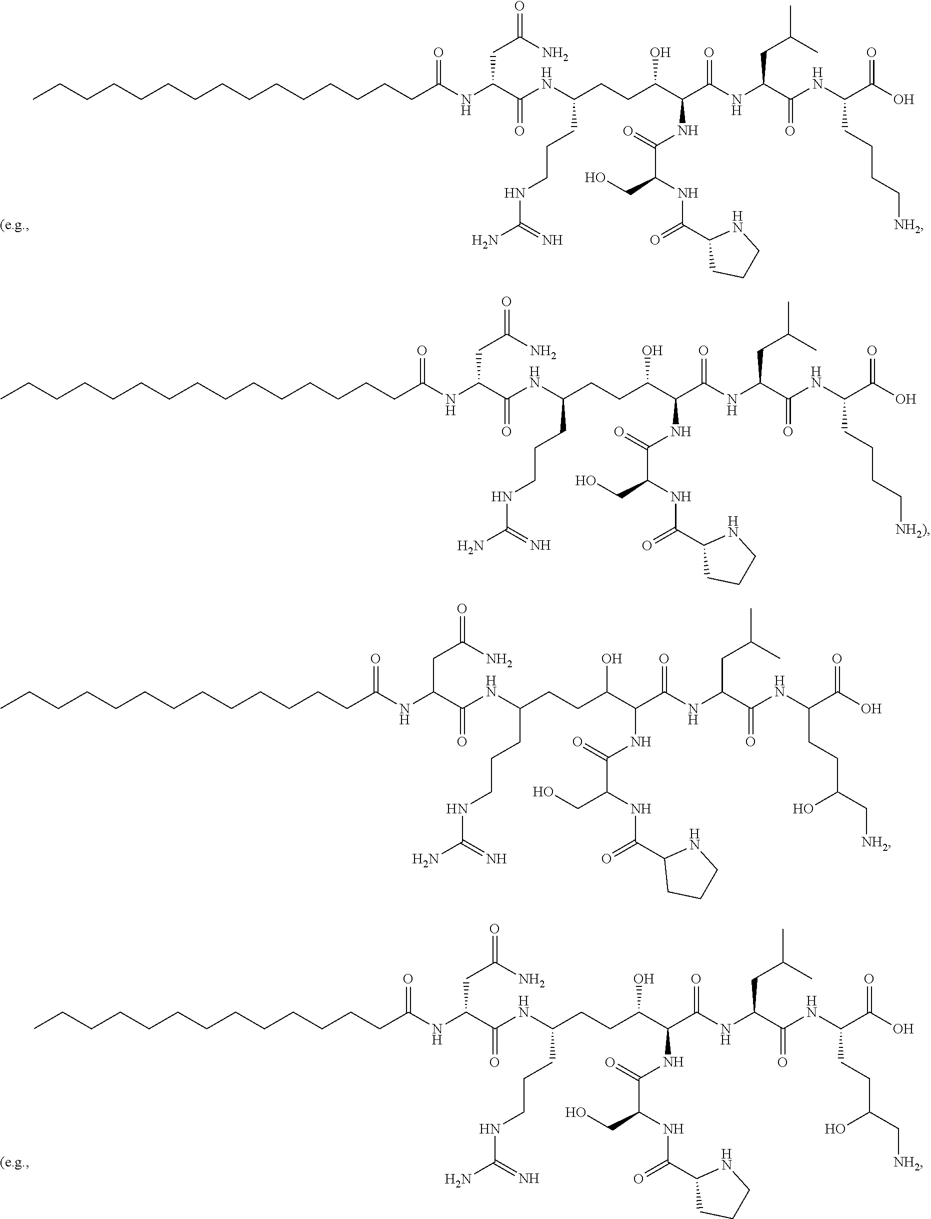

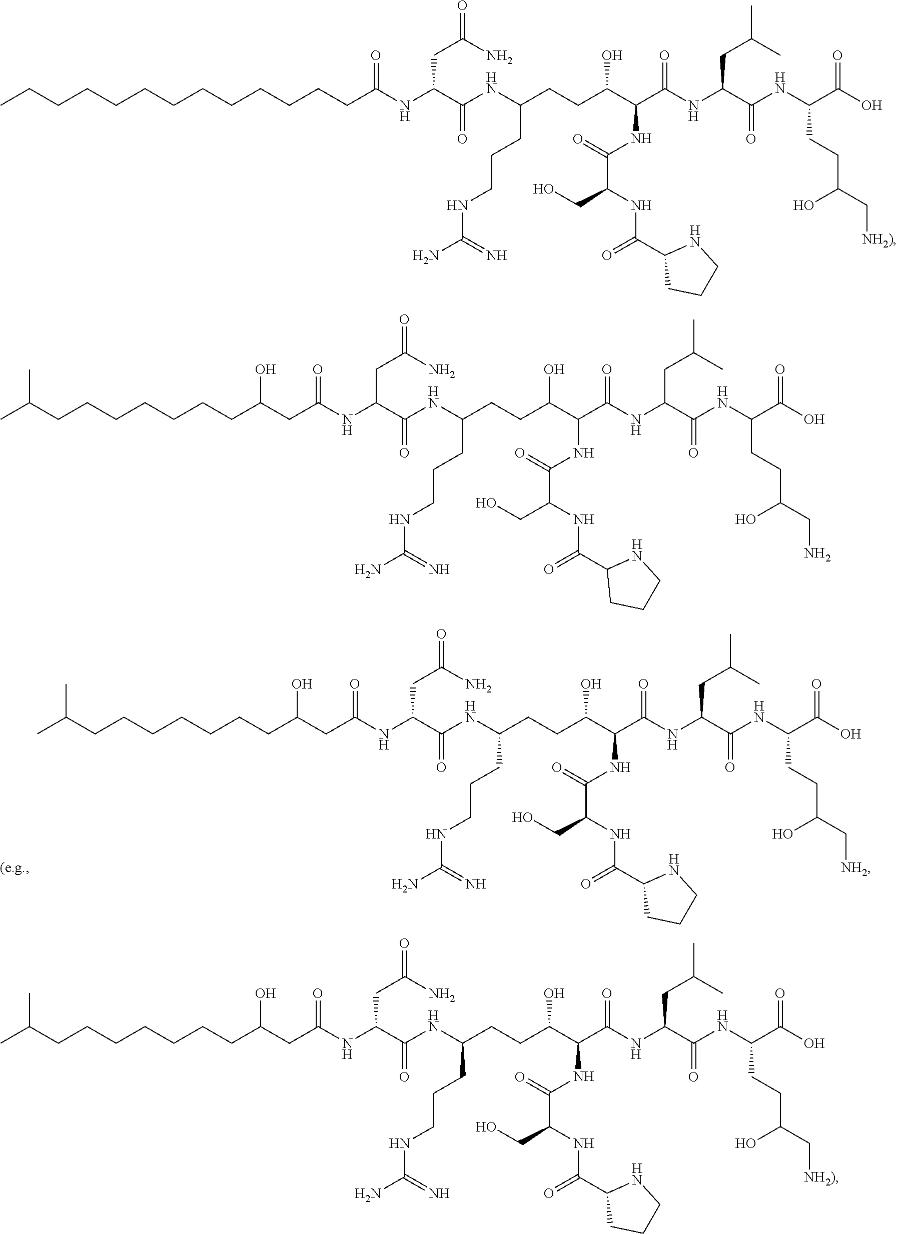

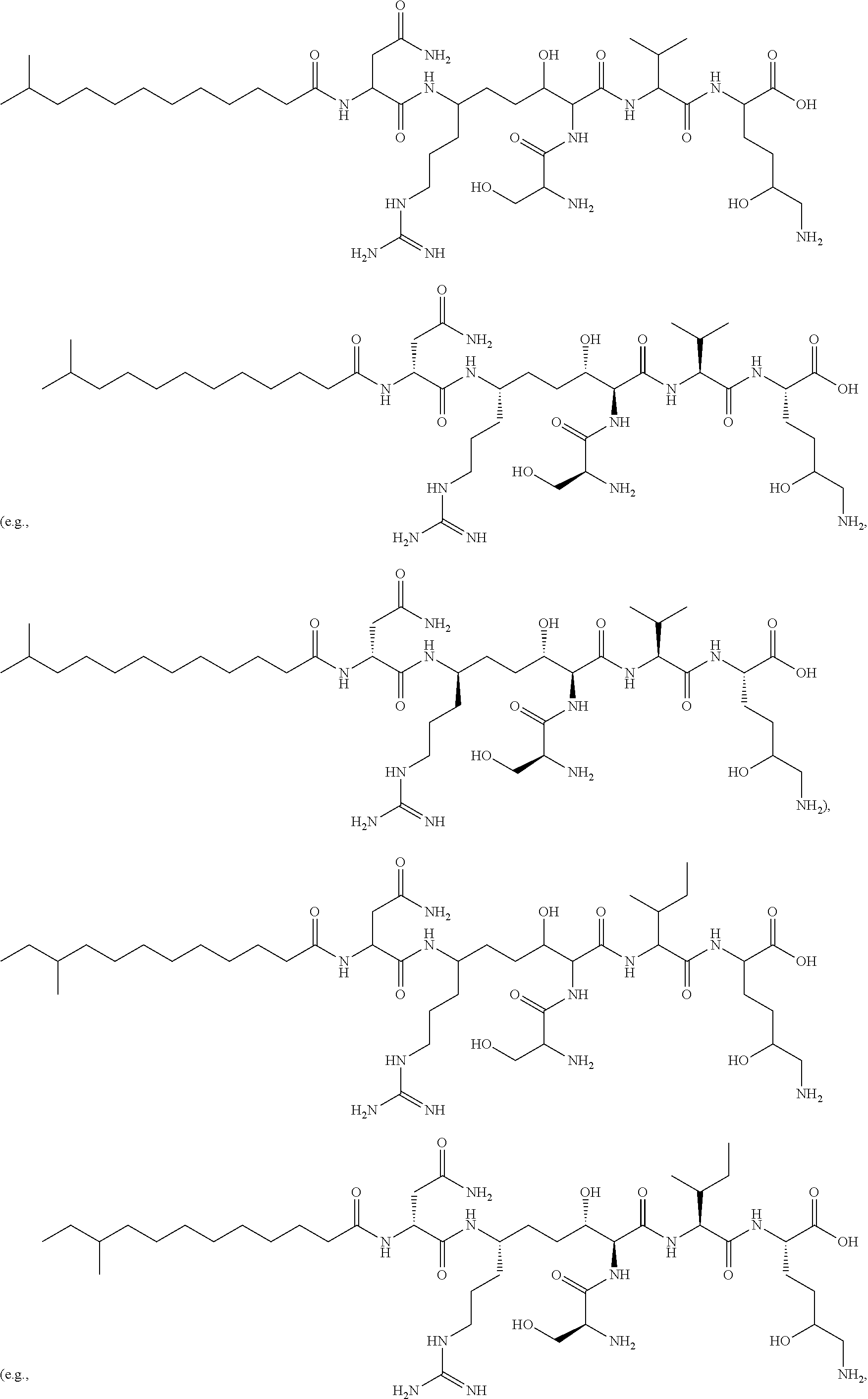

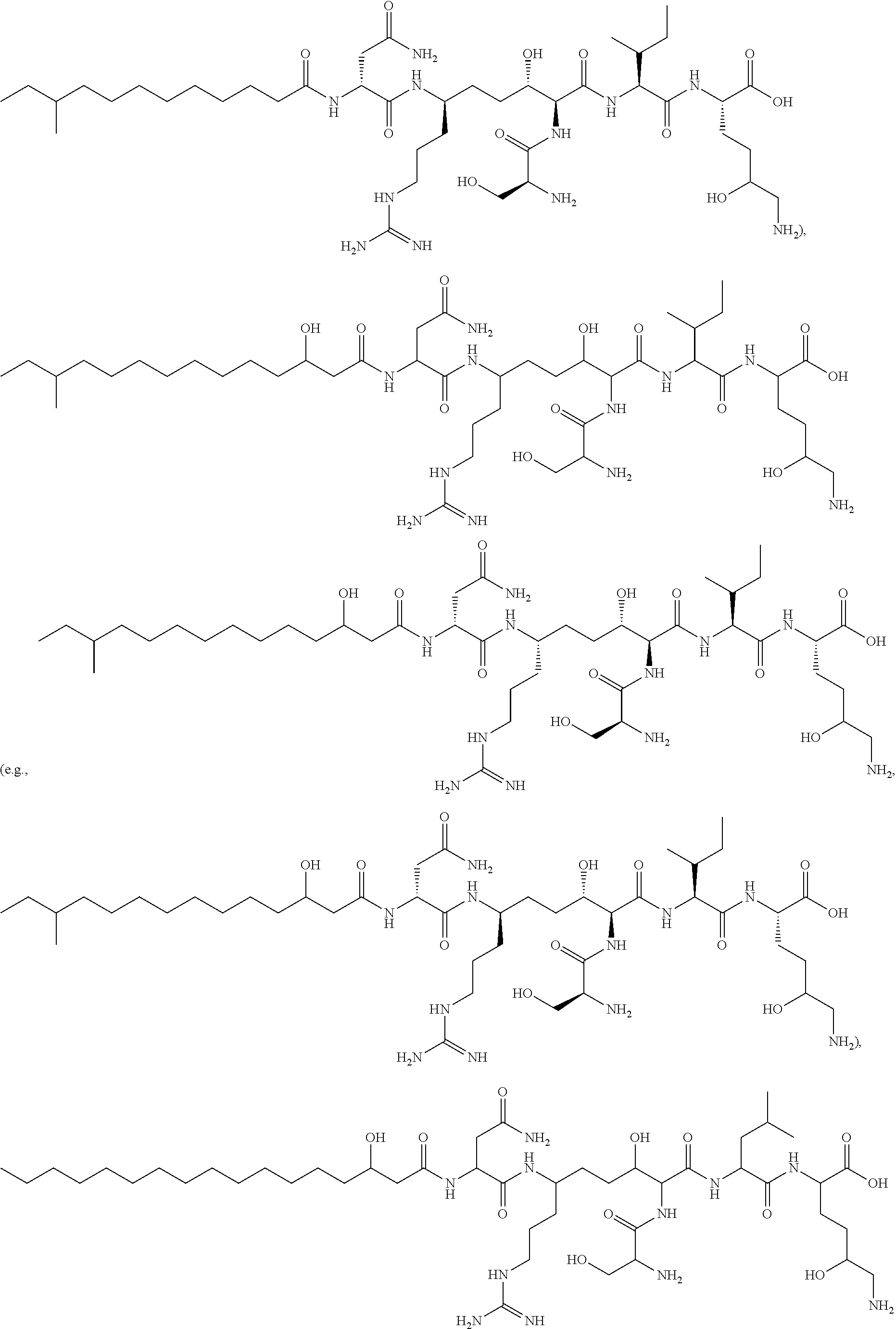

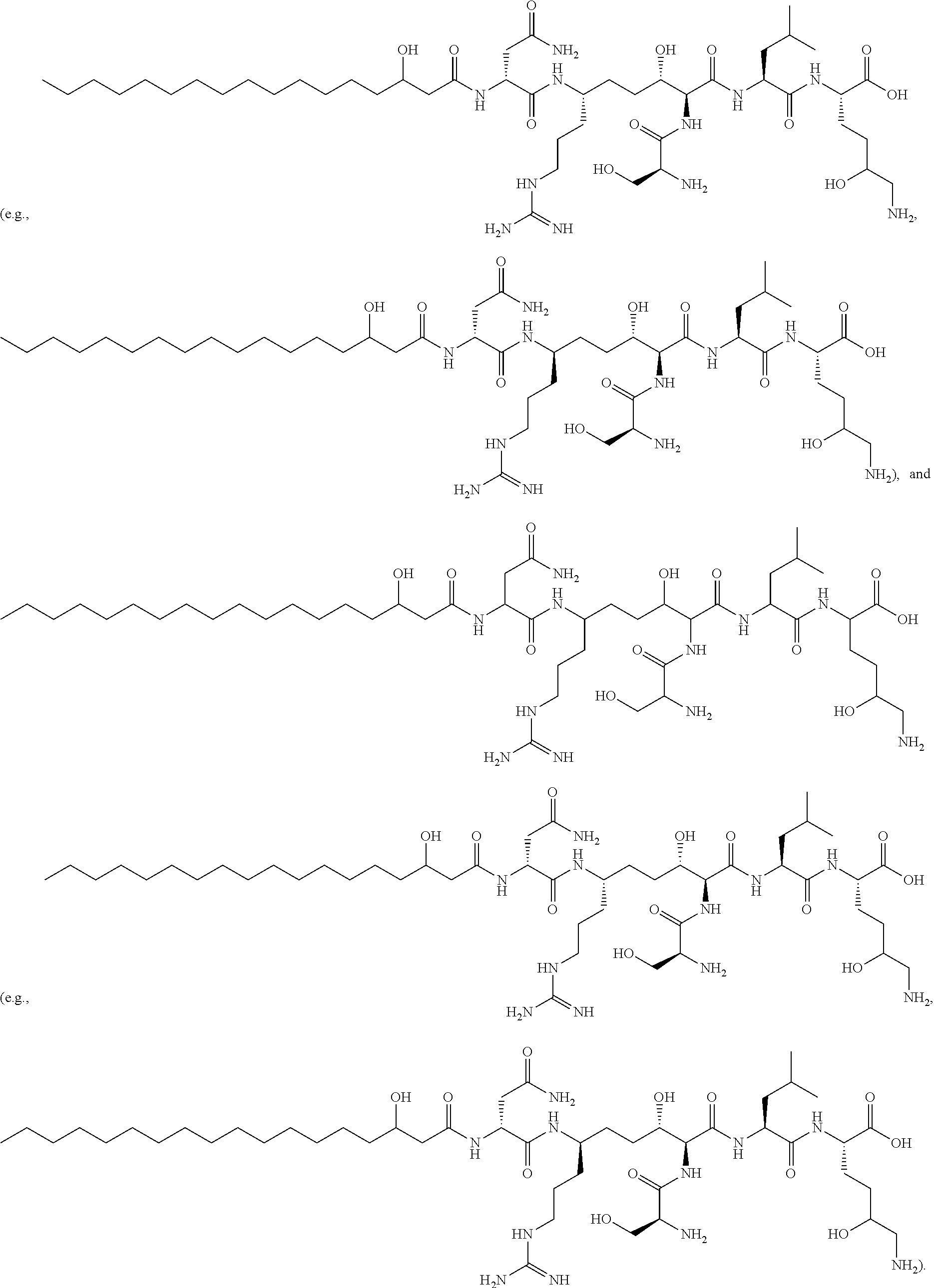

Statement 19. A method according to any one of the preceding Statements, wherein the method is performed in vivo in a subject in need of treatment.
Statement 20. A method according to Statement 19, wherein the subject in need of treatment has colitis, irritable bowel syndrome (IBS), an inflammatory bowel disorder, or has a genetic predisposition to colorectal cancer.
Statement 21. A method according to Statement 19, wherein the method is performed in vivo as a prophylaxis.
Statement 22. A compound having the following structure:

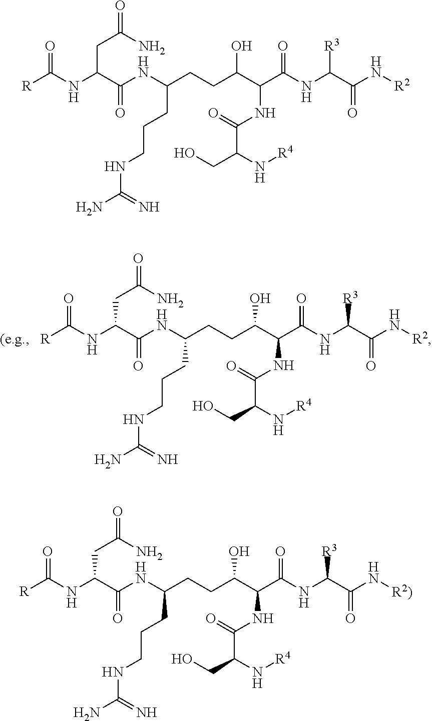

isomers, stereoisomers, enantiomers, diastereomers, salts, hydrates, or solvates of any one or more of the foregoing, or any combination of any of the foregoing, wherein R is a substituted or unsubstituted aliphatic group; R² is an amino acid residue (e.g., a canonical or noncanonical amino acid residue), a substituted or unsubstituted aliphatic group, or H; R³ is a substituted or unsubstituted aliphatic group or an amino acid side chain; and R⁴ is a substituted or unsubstituted acyl group, an amino acid residue, or H, with the proviso the compound does not have the following structure:

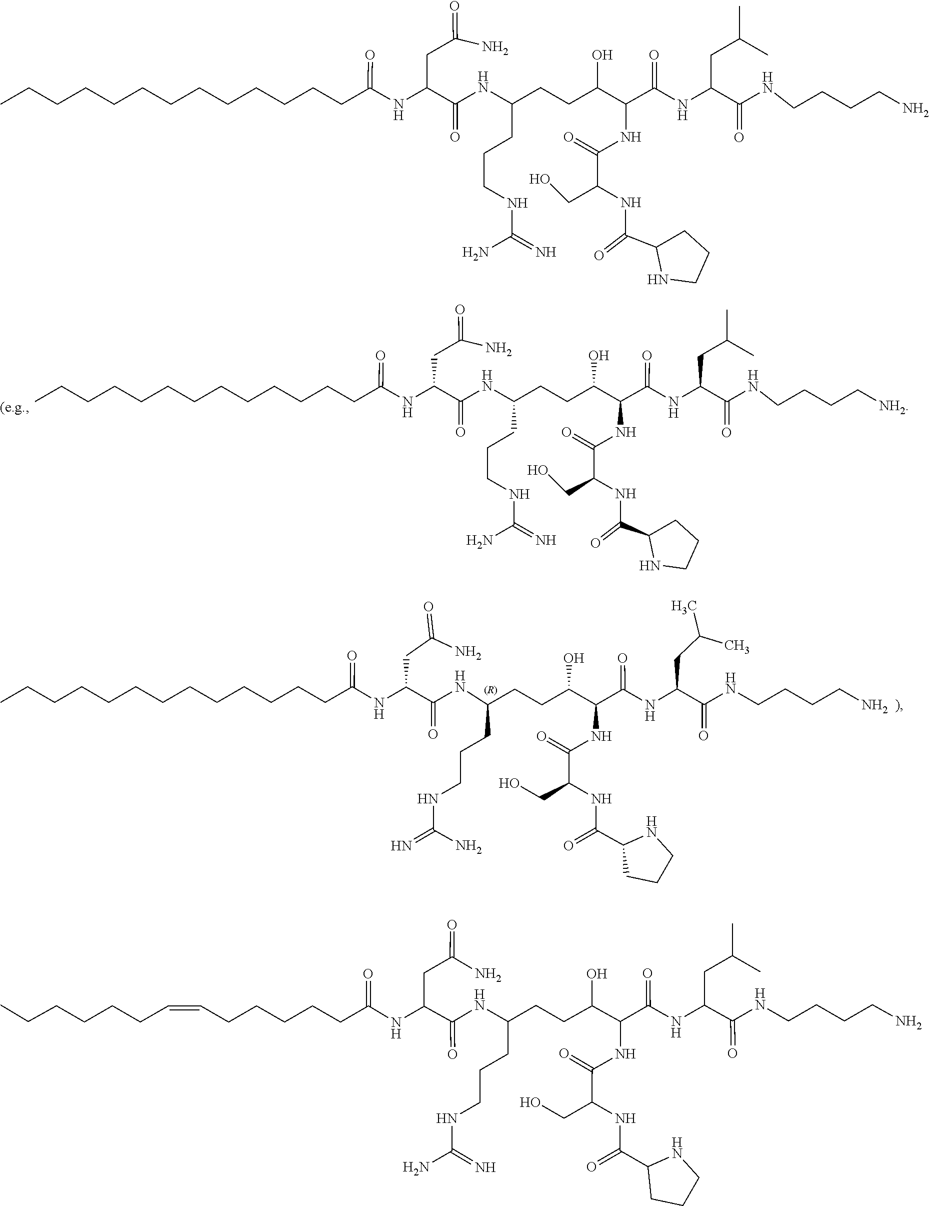

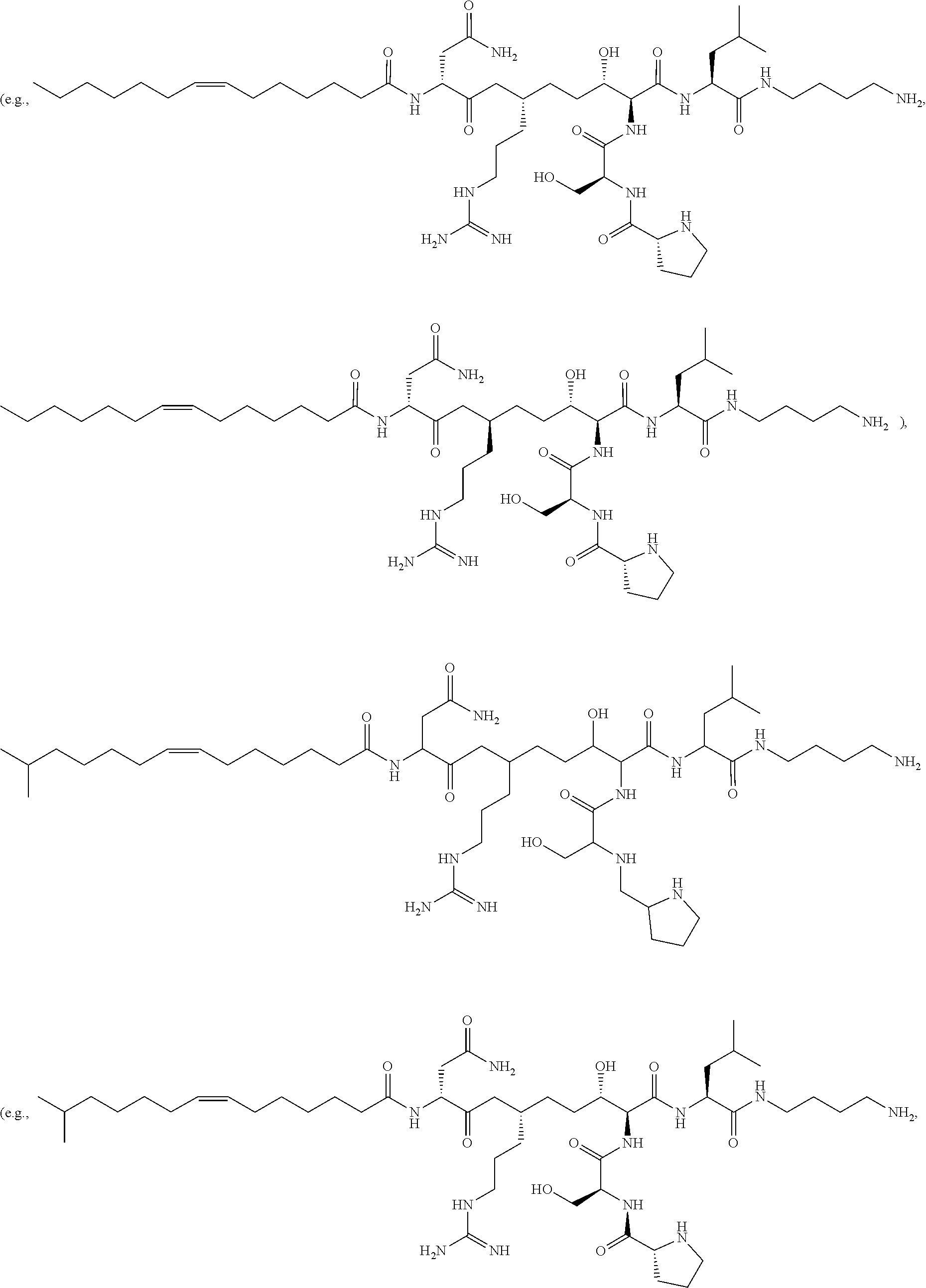

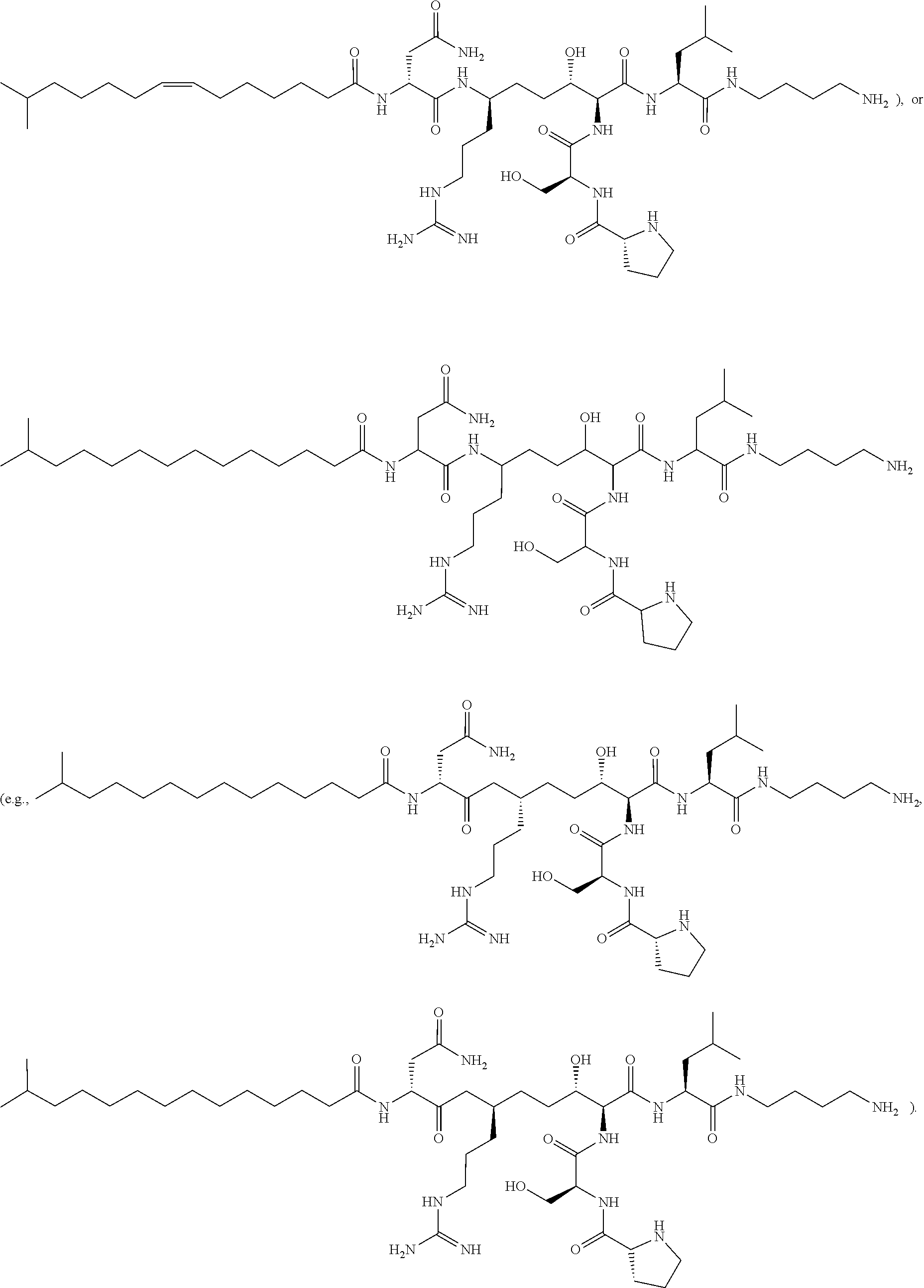

Statement 23. A compound according to Statement 14, wherein the compound has the following structure:

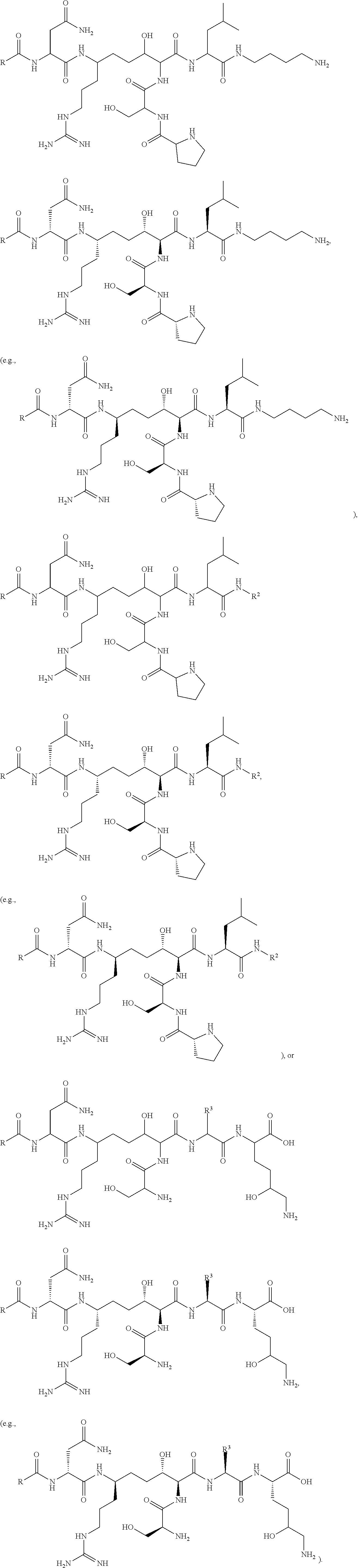

Statement 24. A compound according to claim 22, wherein the aliphatic groups are 1 to 25 carbon atoms in length.
Statement 25. A compound according to Statement 22 or Statement 24, wherein the aliphatic groups further comprise one or more of the following substituents: one or more aromatic rings, one or more pi bonds, one or more hydroxy groups, and/or one or more halogens.
Statement 26. A compound according to Statement 25, wherein the aliphatic groups are substituted with one or more fluoro groups.
Statement 27. A compound according to Statement 22, wherein R has the following structure

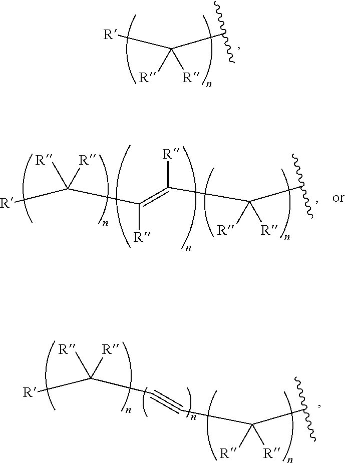

wherein R′ is hydrogen, a halogen (e.g., bromo, fluoro, or the like), an aryl group (e.g., phenyl, napthyl, or the like), a methyl group, halogenated methyl group (e.g., —CF₃), a hydroxy, the like, and each R″ is independently hydrogen, a halogen (e.g., bromo, fluoro, or the like), an aryl group (e.g., phenyl, napthyl, or the like), a methyl group, halogenated methyl group (e.g., —CF₃), a hydroxy, or the like.
Statement 28. A compound according to Statement 23, wherein R has the following structure:

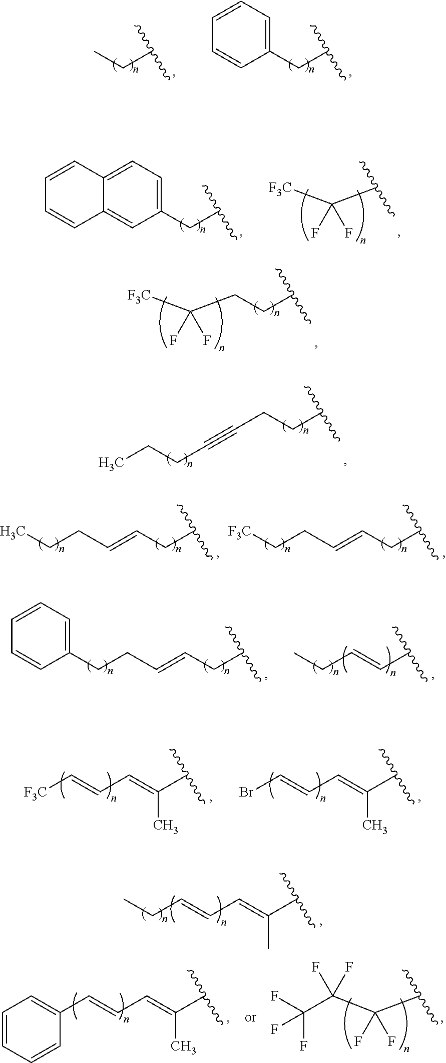

wherein each n is independently 0 to 18 (e.g., 0, 1, 2, 3, 4, 5, 6, 7, 8, 9, 10, 11, 12, 13, 14, 1, 16, 17, 18).
Statement 29. A compound according to Statement 22 or Statement 23, wherein R has the following structure:

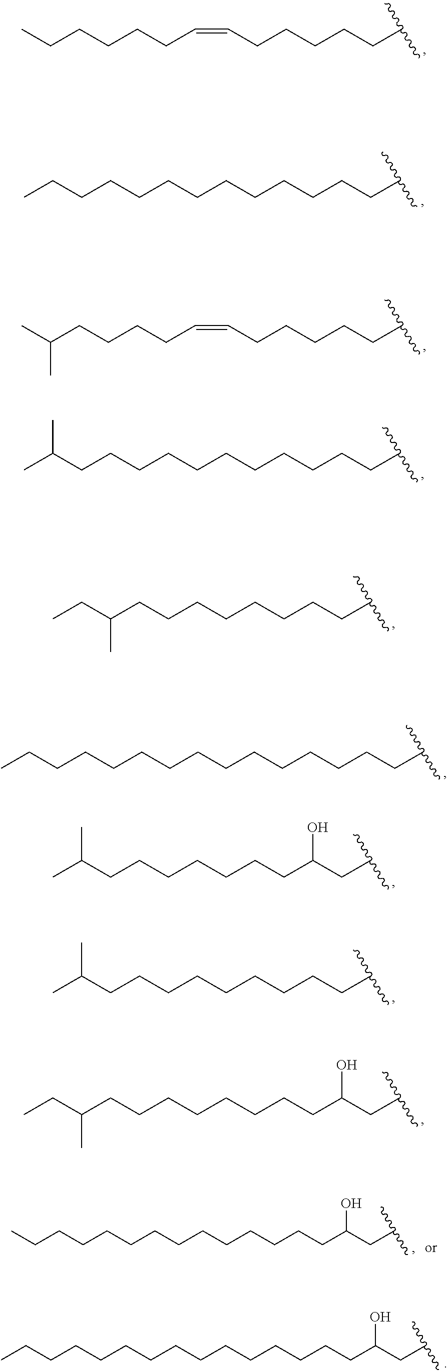

Statement 30. A compound according to any one of Statements 22, 23, or 27 to 29, wherein R² is

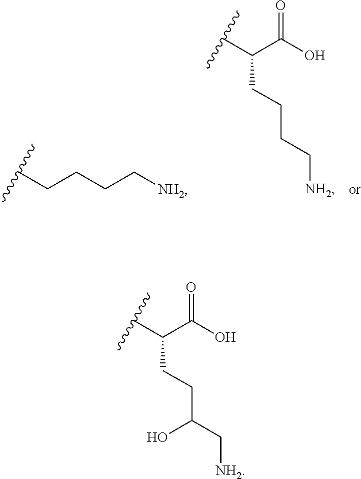 [SMILES: *CCCCN ; *[C@@H](CCCCN)C(=O)O ; *[C@@H](CCC(O)CN)C(=O)O]

Statement 31. A compound according to any one of Statements 22, 23, or 27 to 30, wherein R³ is

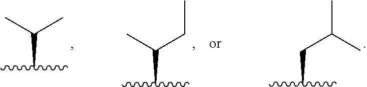

Statement 32. A compound according to any one of Statements 22, 23, or 27 to 31, wherein R⁴ is H or

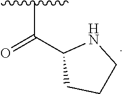 [SMILES: *C(=O)[C@H]1CCCN1]

Statement 33. A composition comprising a compound according to any one of claims 22 to 32.
Statement 34. A composition according to Statement 33, further comprising a pharmaceutically acceptable carrier/excipient.

The following examples are presented to illustrate the present disclosure. They are not intended to be limiting.

### Example 1

This example provides a description of methods and compounds of the present disclosure.

The present disclosure provides using a prodrug antibiotic that is cleaved by ClbP peptidase to cleave to a biologically active drug that selectively kills E coli that produce ClbP.

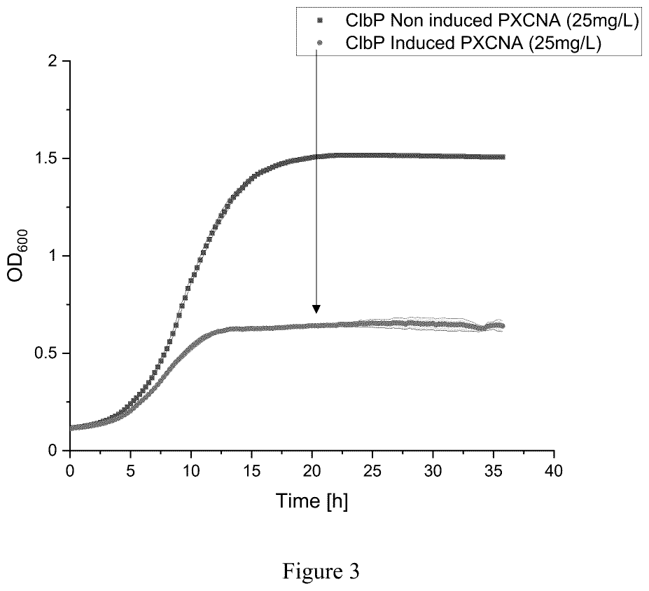

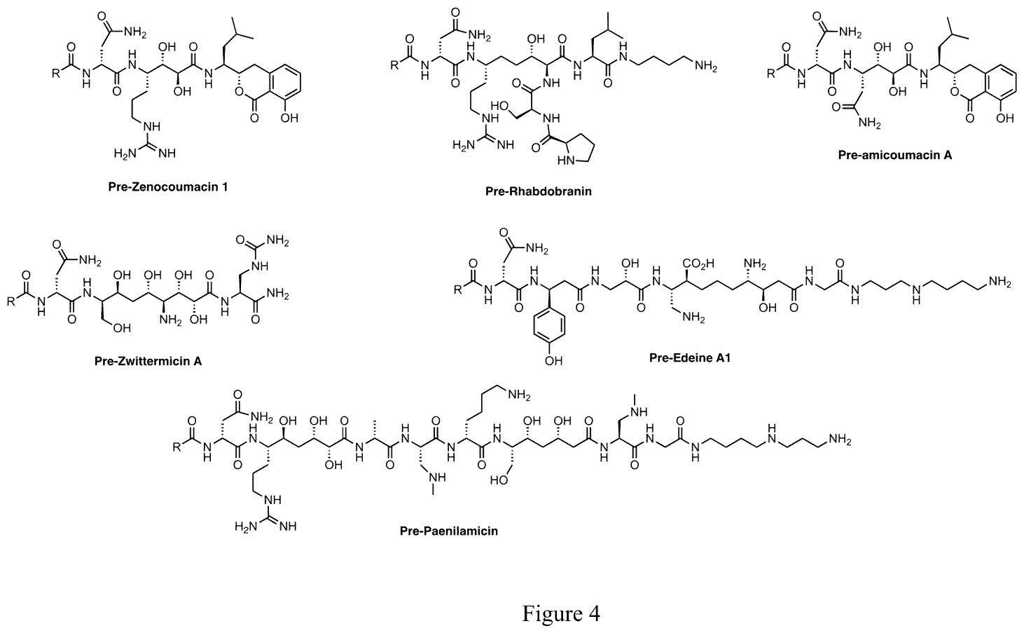

FIG. 3 shows some initial data of prexenocoumacin, an inactive prodrug antibiotic can be taken up and the prodrug cleaved in E. coli expressing the ClbP protease. Prexenocoumacin (PXCNA) kills selectively E. coli expressing the ClbP protease.

ClbP substrate specificity was examined. Cultures were prepared from a single colony from E. coli strain C41(DE3) ΔacrAB pET-29b_clbP strain was inoculated into LB medium supplemented with kanamycin (50 μg/ml). This culture was grown overnight at 37° C. while shaking at 200 RPM. A 1:100 dilution of overnight culture into fresh XPP media supplemented with kanamycin (50 μg/ml) was performed. Peptide solutions were prepared in DMSO and had a final concentration in the culture of 0.01 mM. Samples were taken at T=0, 8 and 24 hrs.

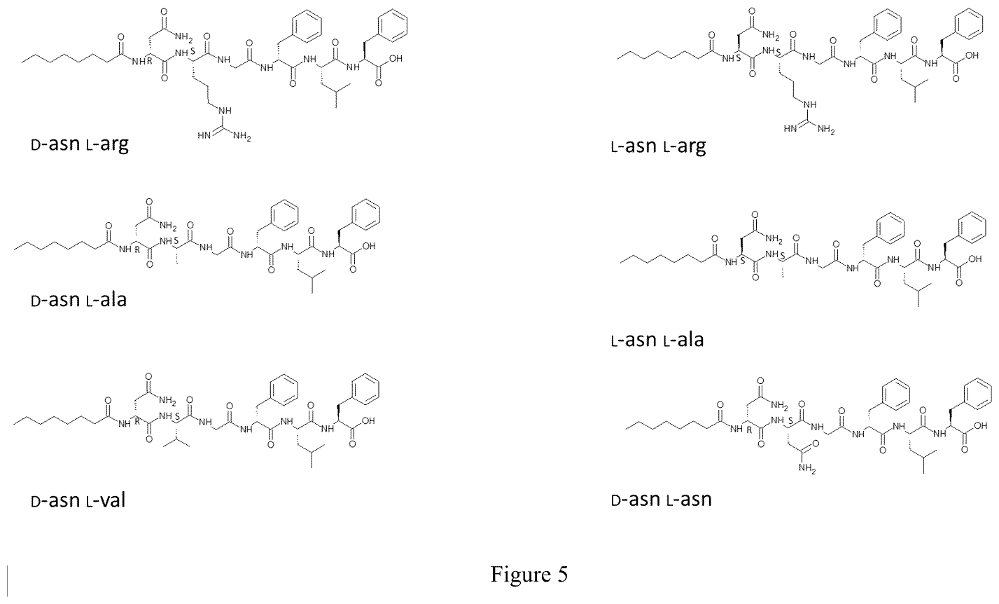

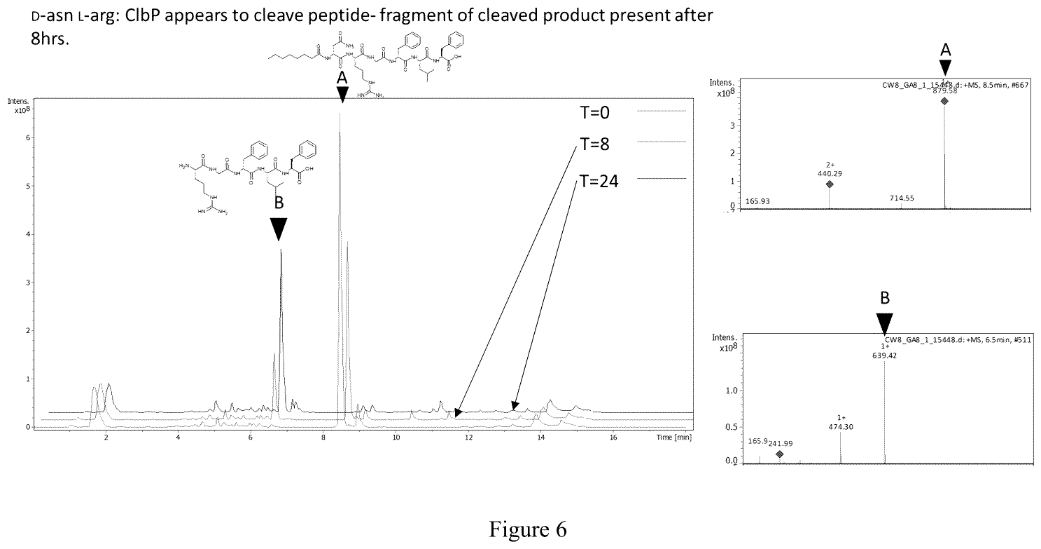

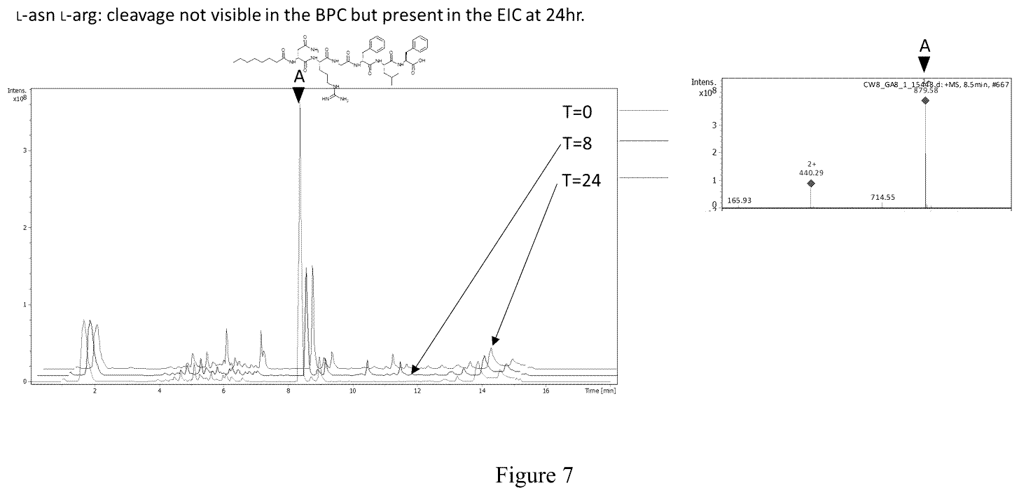

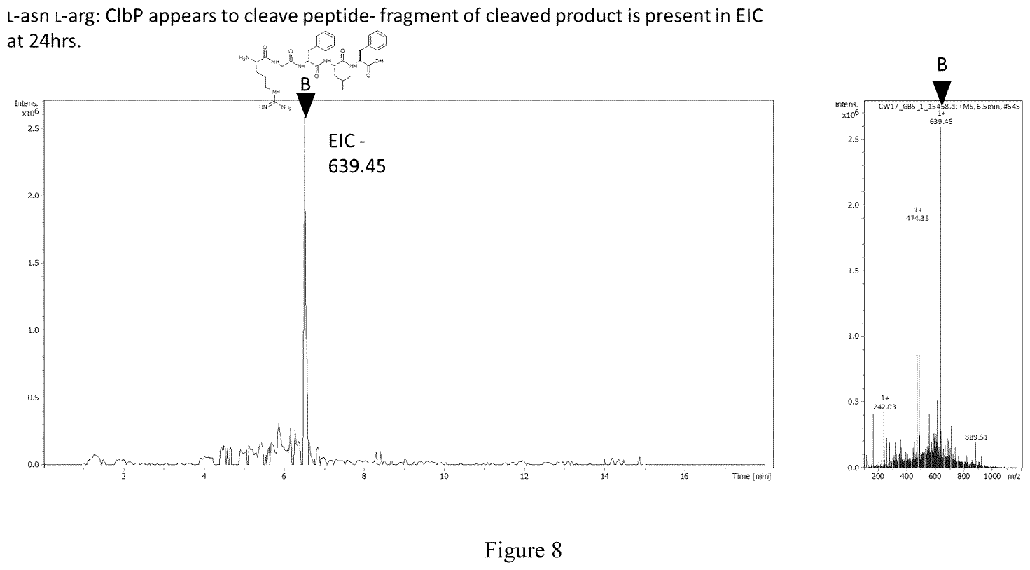

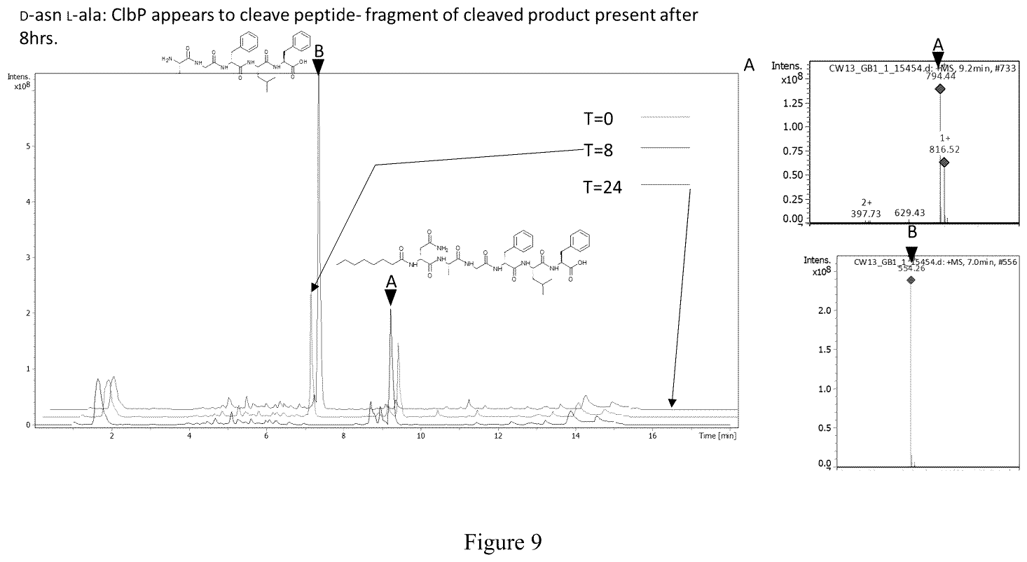

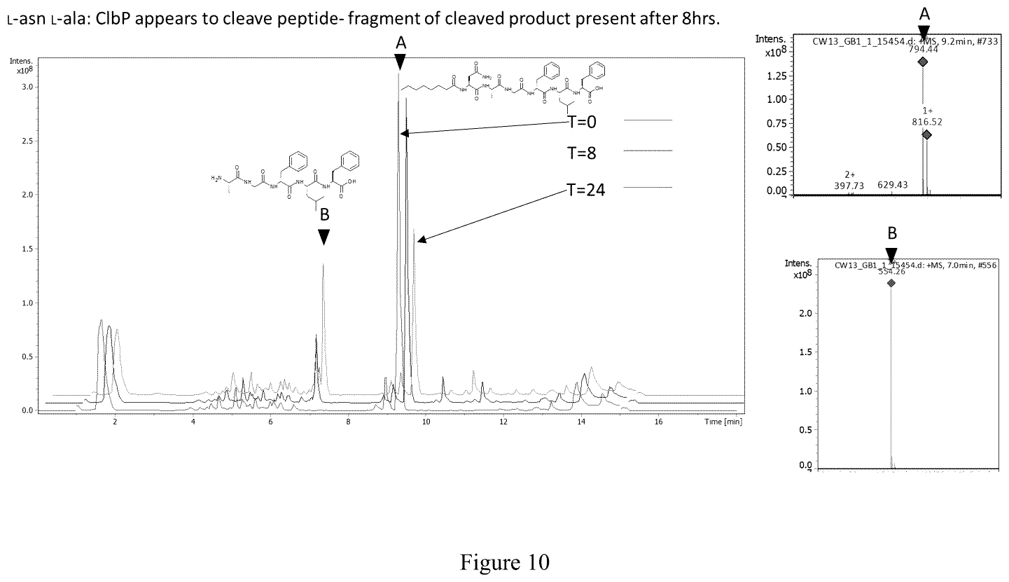

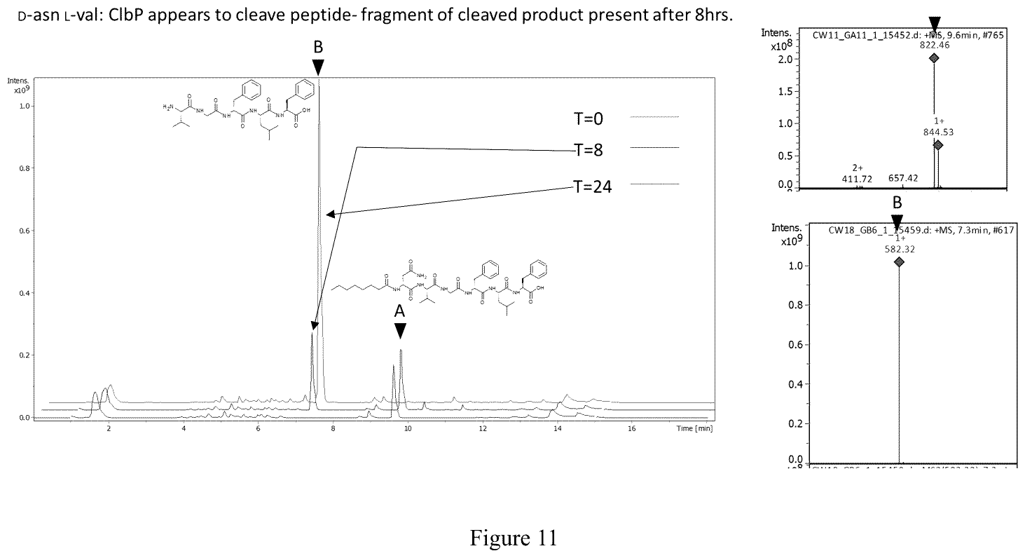

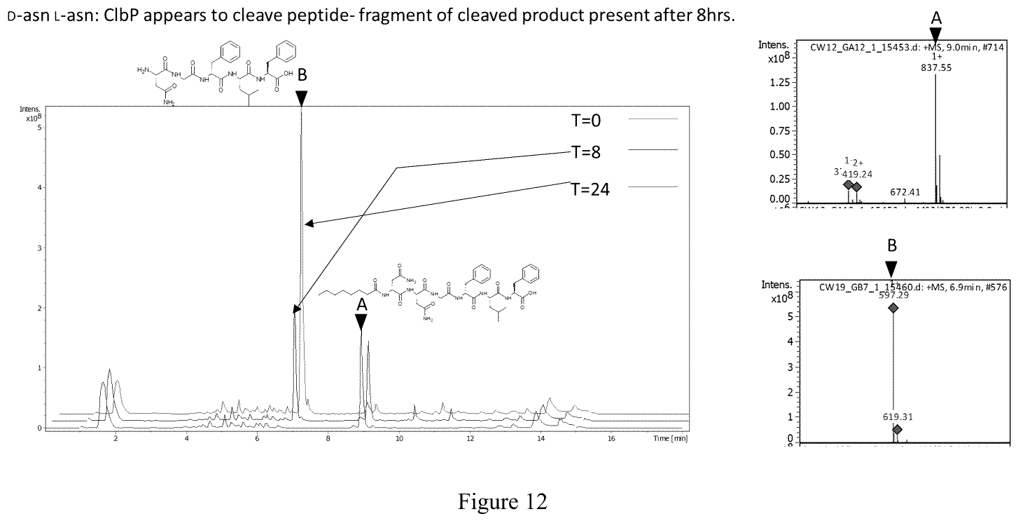

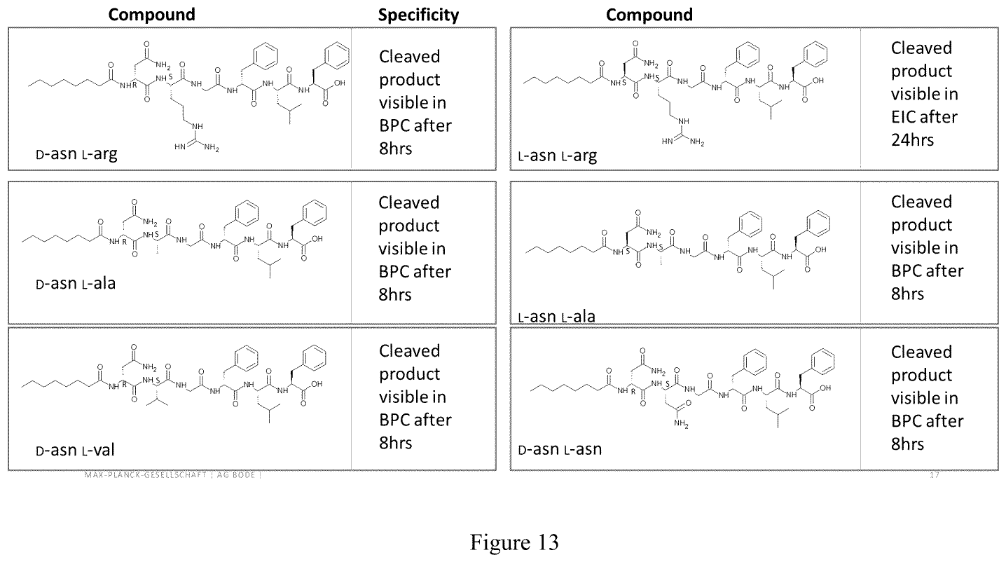

For sampling, a 500 μl aliquot of the culture was taken and had 500 μl of methanol added to it. Samples were shaken for 30 minutes. Samples were centrifuged at 13000 rpm for 20 minutes then 100 μl of supernatant was assayed by HPLC-MS (AMAZON system). These data are in FIGS. 6 to 13. The sample substrates are shown in FIG. 5.

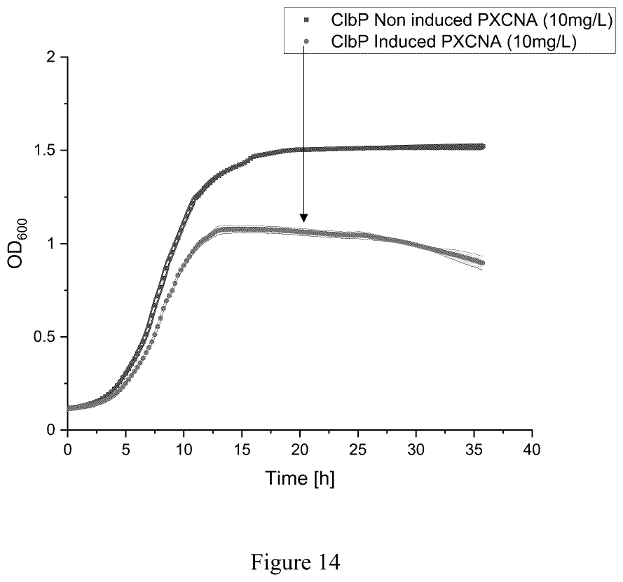

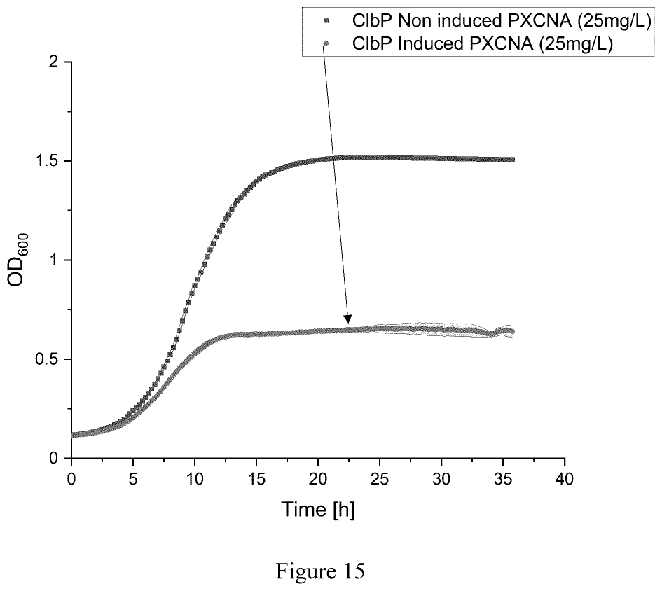

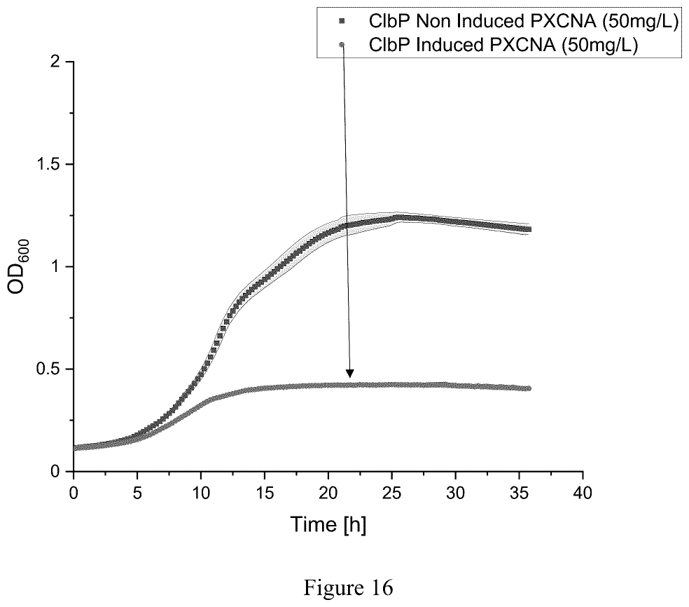

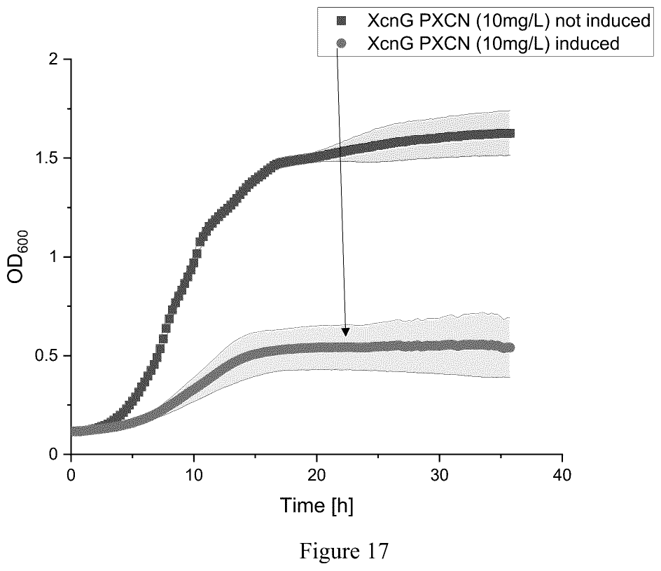

Growth curves made using PXCNA. Briefly, a single colony from E. coli strain C41(DE3)_ΔacrABpET-29b clbP strain was inoculated into LB medium supplemented with kanamycin (50 μg/ml). This culture was grown overnight at 37° C. while shaking at 200 RPM. 1:100 dilution of overnight culture into fresh LB medium supplemented with kanamycin (50 μg/ml) was performed. Samples were induced with IPTG (0.5 mM). A crude extract of PXCNA prepared in methanol (1 mg/ml). The final concentration in samples was 50 mg/L, 25 mg/L and 10 mg/L. Samples were incubated at 22° C. for 24 hrs and OD₆₀₀ was measured using TecanSpark. These data are found in FIGS. 14 to 17.

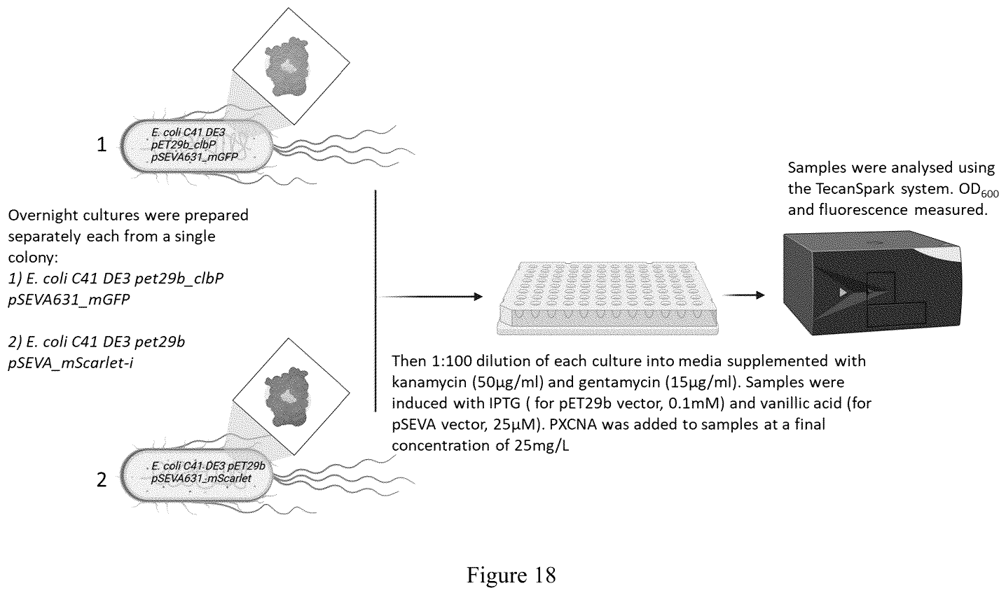

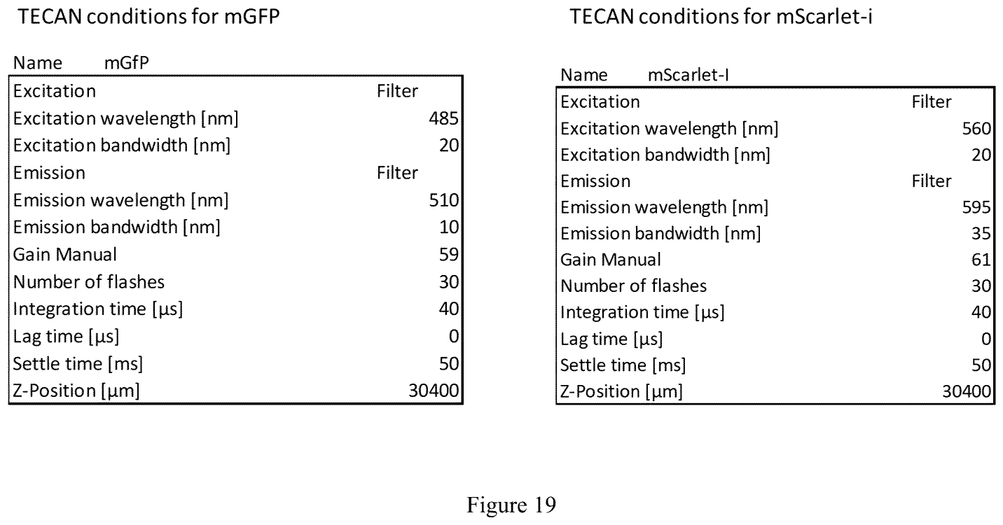

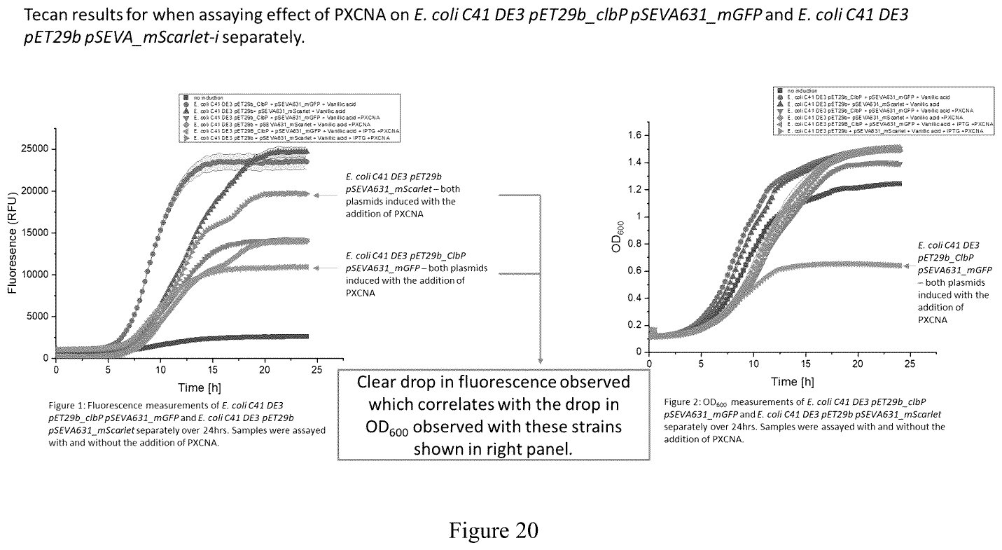

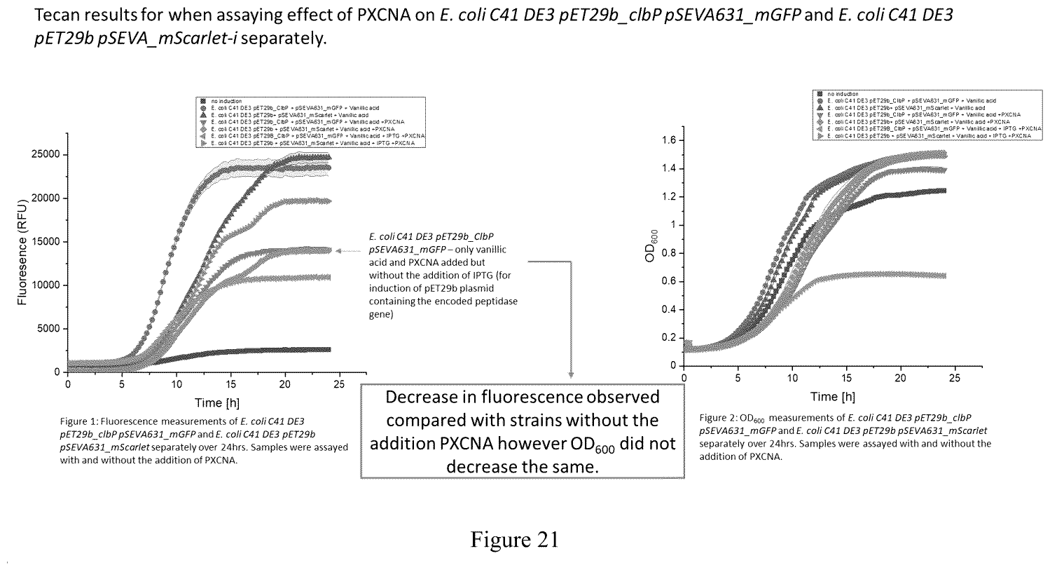

PXCNA activity was measured via fluorescence (see FIGS. 18 and 19). These data are found in FIGS. 20 and 21.

A co-culture assay was performed. Overnight cultures of E. coli C41 DE3 pet29b_cIbP pSEVA631_mGFP and E. coli C41 DE3 pet29b pSEVA_mScarlet-i were prepared separately each from a single colony. Then 1:100 dilution of each culture into the same LB media well supplemented with kanamycin (50 μg/ml) and gentamycin (15 μg/ml) was performed. Samples were induced with IPTG (for pET29b vector, 0.1 mM) and vanillic acid (for pSEVA vector, 25 μM). Samples were fed with PXCNA with a final concentration of 25 mg/L.

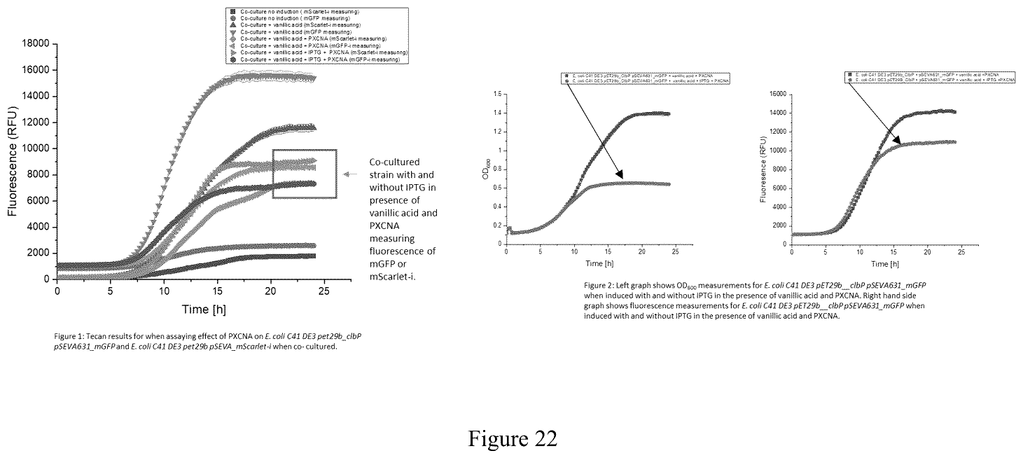

In FIG. 22, a low level of fluorescence observed for both the mGFP reading (corresponds to strain with ClbP) and mScarlet-i reading (corresponds to strain with empty vector) when co-culture is induced with or without IPTG (inducing peptidase containing plasmid), when in the presence of PXCNA and vanillic acid (inducing fluorescent protein containing plasmid). The drop in fluorescence could be because once the prexenocoumacin A is cleaved forming xenocoumacin I, some of this is being exported out of the cell and inhibiting the growth of the empty vector strain, thus reducing its fluorescence. However, when there is no IPTG present the same result is observed potentially meaning there is leaky expression of the T7 promoter. However, when measuring the OD₆₀₀ of E. coli C41 DE3 pet29b_clbPpSEVA631_mGFP with only the addition of vanillic acid and PXCNA, the growth did not appear inhibited despite a drop in fluorescence, so it seems unlikely there is significant leaky expression of the peptidase (FIG. 22, right panels).

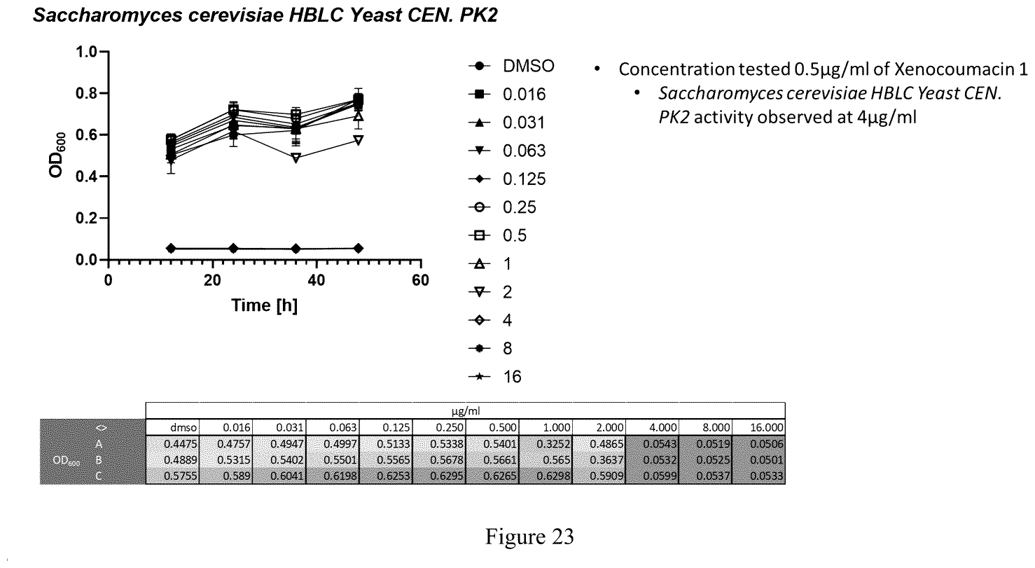

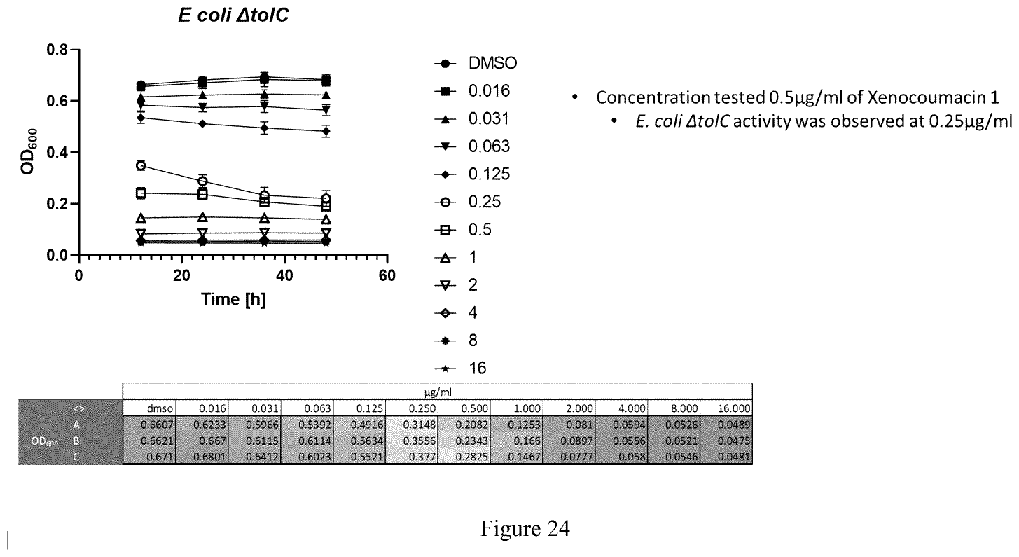

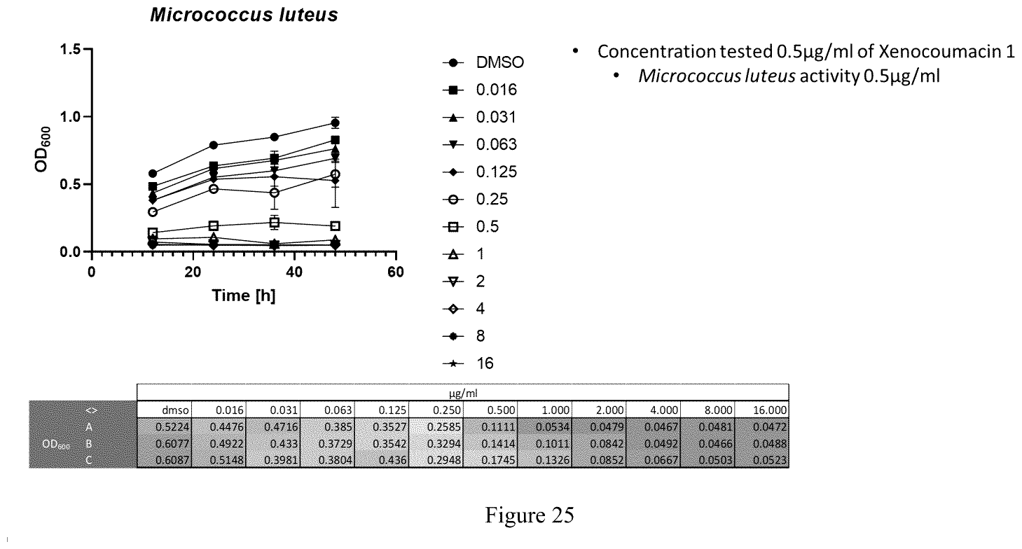

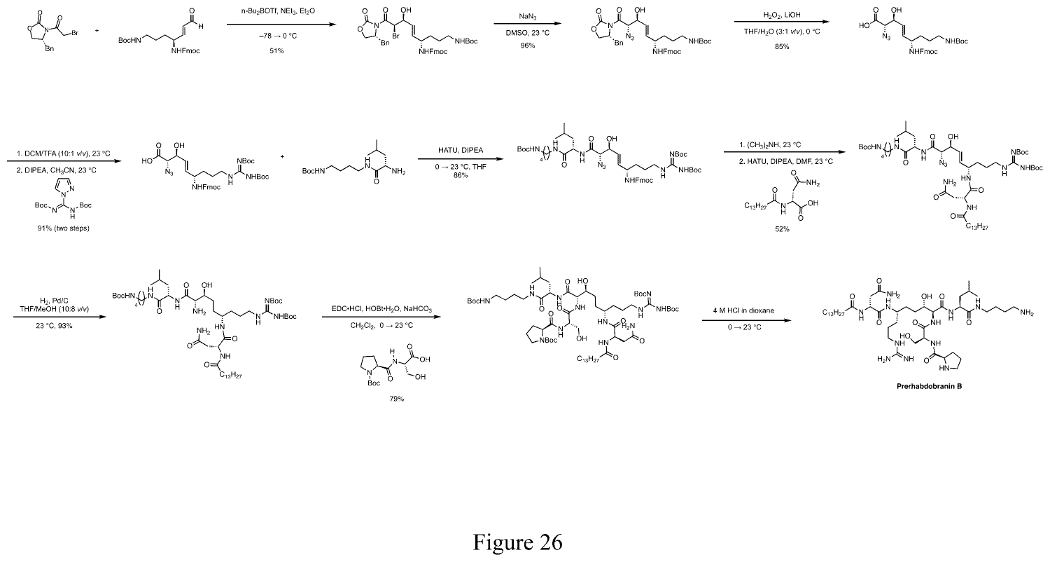

Minimum inhibitory concentration (MIC) assays were also performed. A single colony of each strain inoculated into fresh LB media and grown overnight at 30° C. and shaken at 200 rpm. Overnight cultures were diluted to OD₆₀₀=0.06. Cultures were inoculated with test compounds at OD₆₀₀=0.06. A 96-well plate was incubated at 30° C. and 700 rpm. The compound stock solutions prepared in DMSO. These data are in FIGS. 23 to 25.

### Example 2

This example provides a description of methods and compounds of the present disclosure.

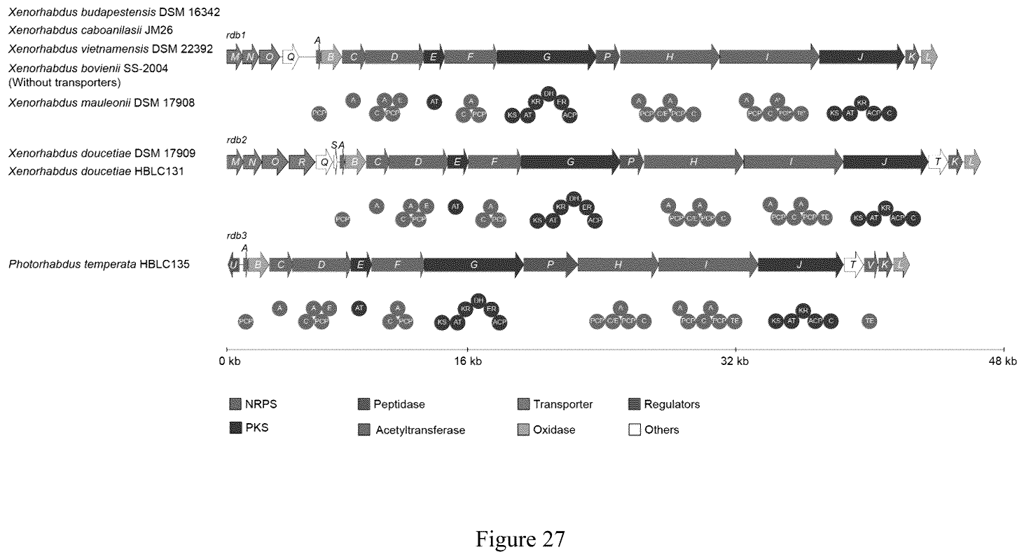

Results: NRPS-PKS Hybrid BGC encoding the biosynthesis of rhabdobranin. Three types of NRPS-PKS hybrid BGCs encoding the biosynthesis of rhabdobranins were found in a Photorhabdus and various Xenorhabdus strains by applying a combination of pangenomic and domain sequence similarity network approaches. The clusters are of interest as they encode a peptidase, suggested to be linked to a self-resistance mechanism (FIG. 27). Comparable peptidases were previously described in the biosynthesis of potent antimicrobial compounds, such as amicoumacin and xenocoumacin, inhibiting messenger RNA translation or colibactin, a genotoxin alkylating DNA, which led to the hypothesis that rhabdobranin might also show antimicrobial properties.

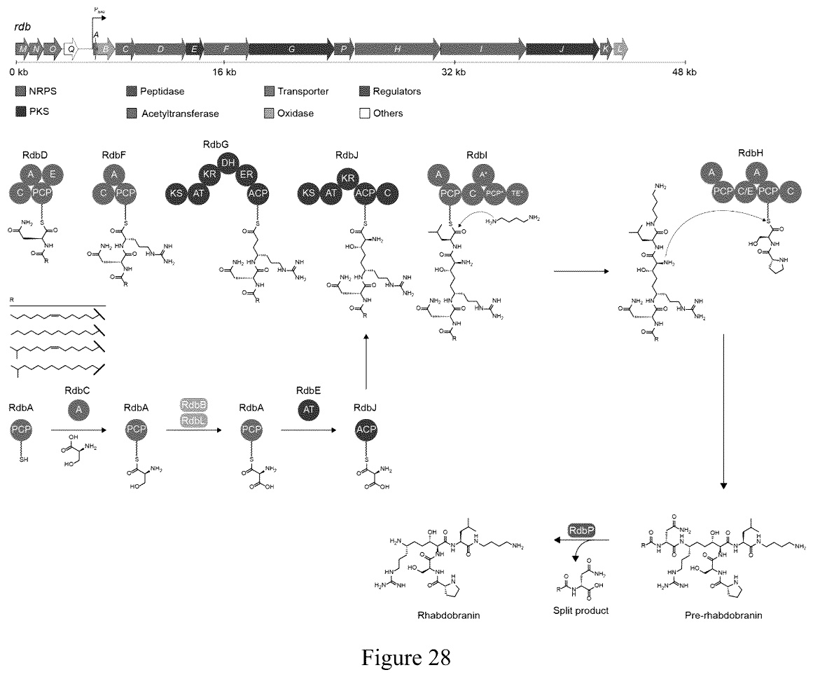

The mechanism of the rhabdobranin biosynthesis in Xenorhabdus budapestensis DSM 16342 was proposed, under consideration of antiSMASH predictions, HPLC-HR-MS analysis of generated deletion mutants and promoter exchange variants, as well as structural analysis using nuclear magnetic resonance spectroscopy (NMR) (FIG. 28). The synthesis was suggested to start with the generation of an acylated

D-asparaginyl moiety with four possible lipid residues, resulting in the prodrug molecules pre-rhabdobranin A, B, C and D. The peptidase, likely anchored in the inner membrane of the Gram-negative strains was suggested activating the prodrug molecules in the periplasm, by catalyzing the release of the N-terminal acylated D-asparaginyl moiety.

Characterization of rhabdobranin. While the prodrug molecules pre-rhabdobranin A to D from Xenorhabdus budapestensis DSM 16342, were structurally characterized, and the production of respective derivatives in rdbP deletion mutants of Photorhabdus temperata HBLC135 and Xenorhadbdus doucetiae DSM 17909 was activated previously through promoter exchange strategies, the hydrophilic rhabdobranin molecule was previously undetected. As structural predictions relied on MS data, under consideration of the detected split product, a crucial need remained to validate the structure of rhabdobranin and its proposed antimicrobial properties. In this part of my project, I detected the molecule in Xenorhabdus budapestensis DSM 16342 and devised an optimized in vitro conversion method coupled with a refined isolation strategy to facilitate the structural characterization of the compound. Additionally, the compound's antimicrobial activity has been analyzed and investigations for the drug target were conducted.

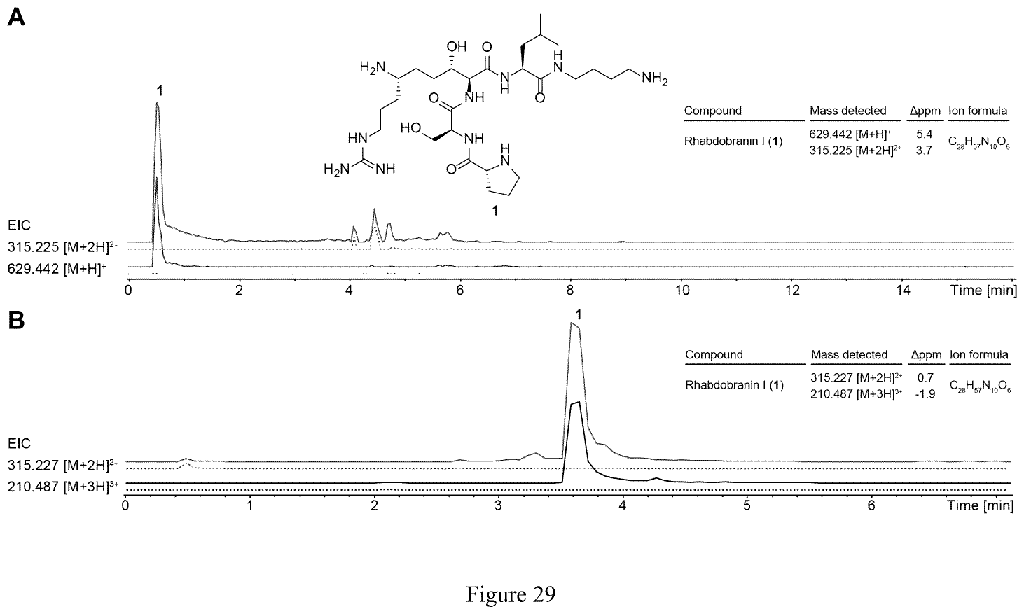

Detection of rhabdobranin I in Xenorhabdus budapestensis DSM 16342. For the detection of rhabdobranin I in a Xenorhabdus budapestensis DSM 16342 pCEP_kan-rdbA promoter exchange mutant, an HPLC-HR-MS analysis was performed using a C18 column (FIG. 29A), and an amide column for hydrophilic interaction chromatography (HILIC) (FIG. 29B). This led to the detection of the predicted single ion mass, as well as the corresponding double- and triple-charged ions of rhabdobranin I (1) (Table 5, (Supplements)), in the 0.4% L-arabinose induced promoter exchange variant, verifying the proposed prodrug activation catalyzed by RdbP. While the single-charged ion was only detected using C18 separation, and the triple-charged ion was only detected using HILIC separation, the double-charged ion was detected under both conditions.

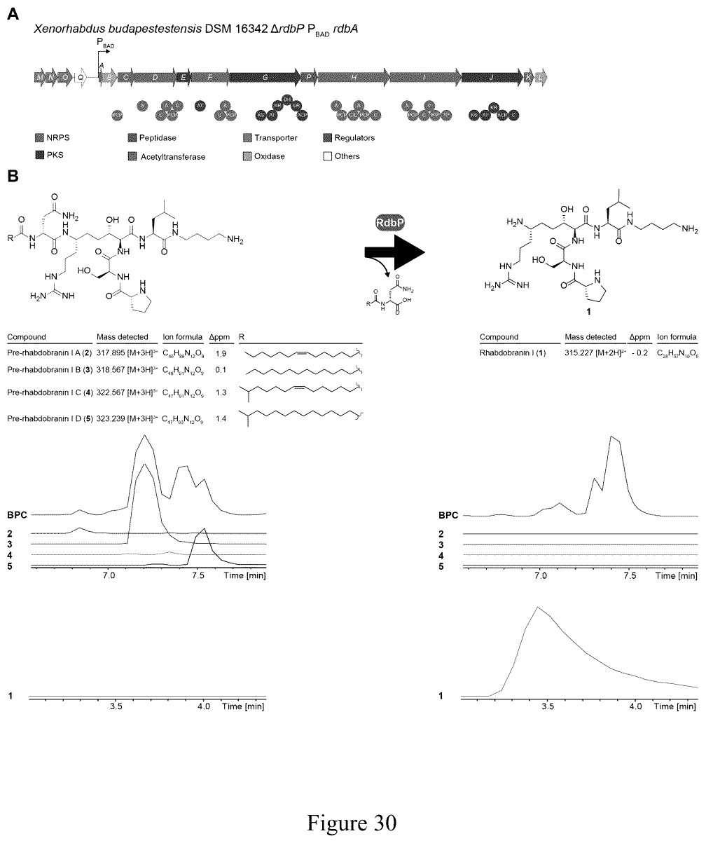

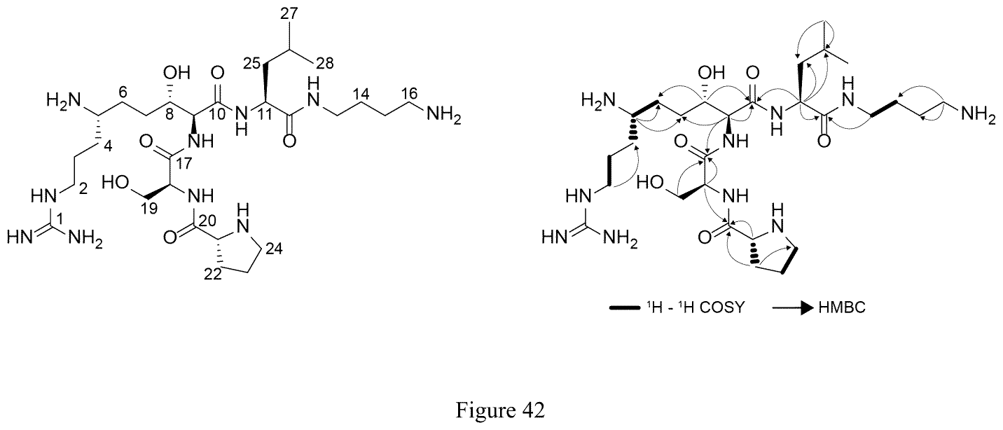

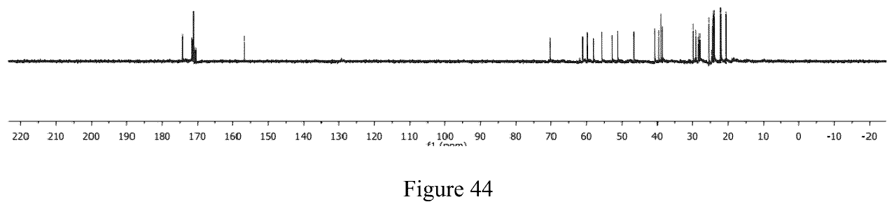

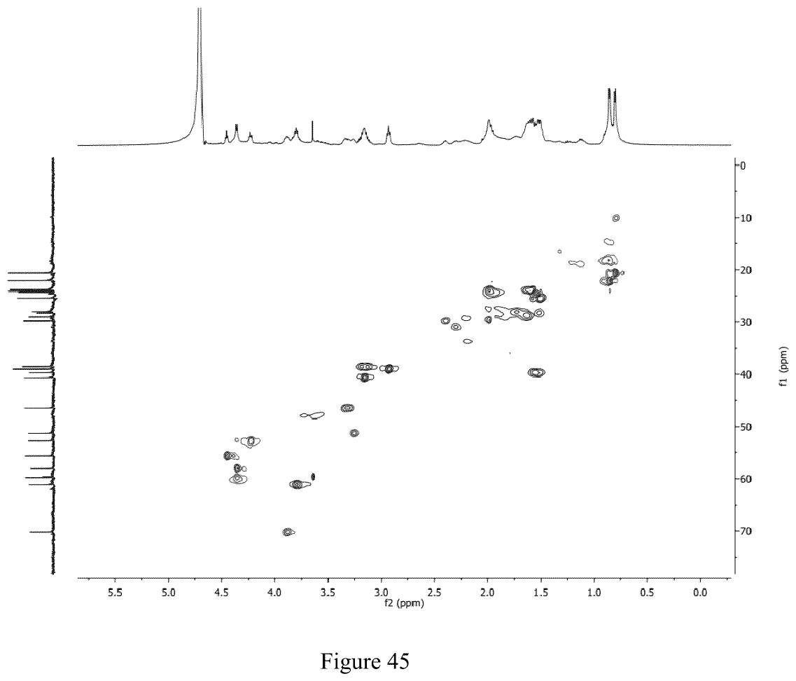

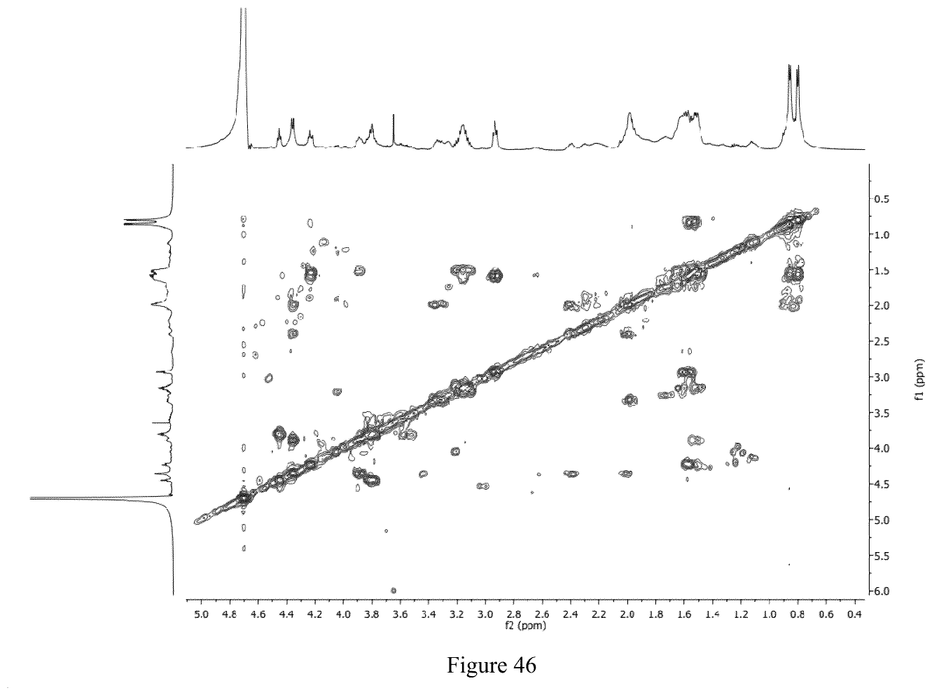

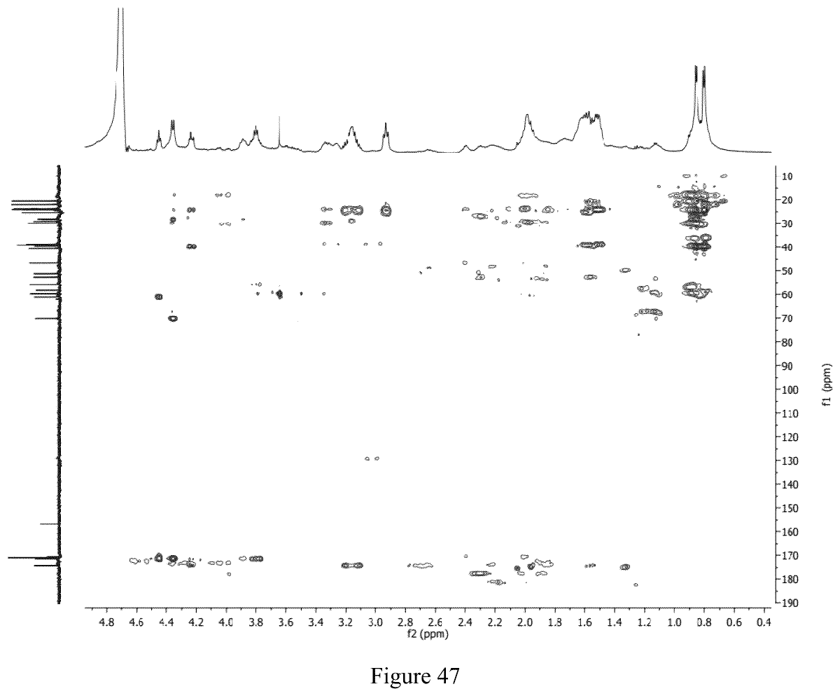

Large-scale production of rhabdobranin I for bioactivity tests and structural analysis. To optimize the isolation of the polar rhabdobranin I molecule, a large-scale in vitro conversion of the prodrug molecule was conducted. Therefore, a pre-rhabdobranin I A, B, C and D (2, 3, 4 and 5) containing fraction was isolated from a Xenorhabdus budapestensis DSM 16342 ΔrdbP pCEP_kan-rdbA production culture (FIG. 30A). The extract was converted under addition of prepared Escherichia coli membrane containing the RdbP peptidase. A subsequent HILIC isolation of the resulting rhabdobranin I (1) was performed (FIG. 30B). The isolated compound was used for the structure verification by NMR (FIG. 42-47, Table 6, (Supplements)) and bioactivity assays.

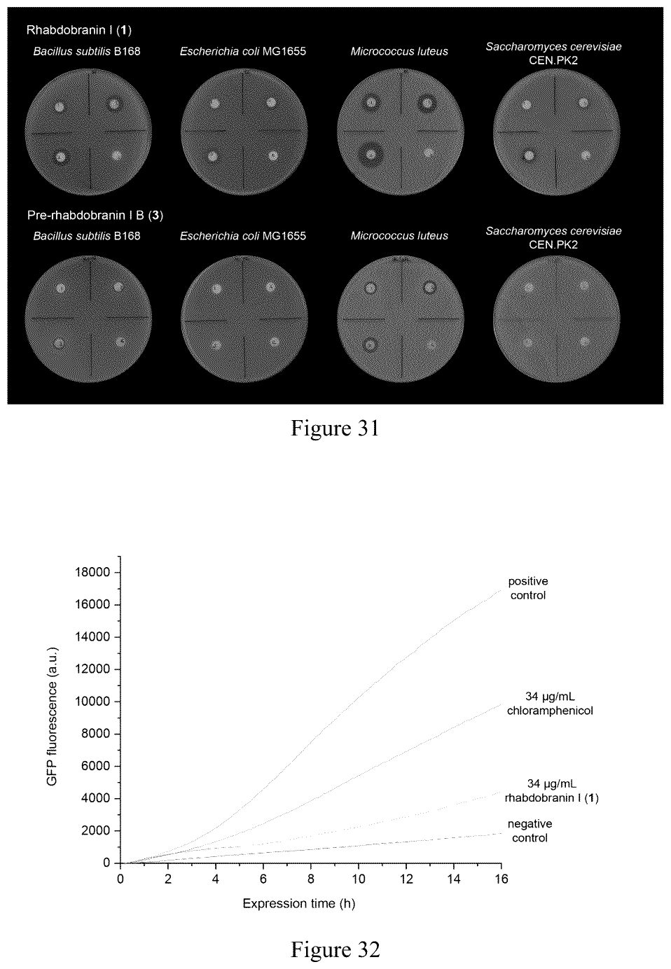

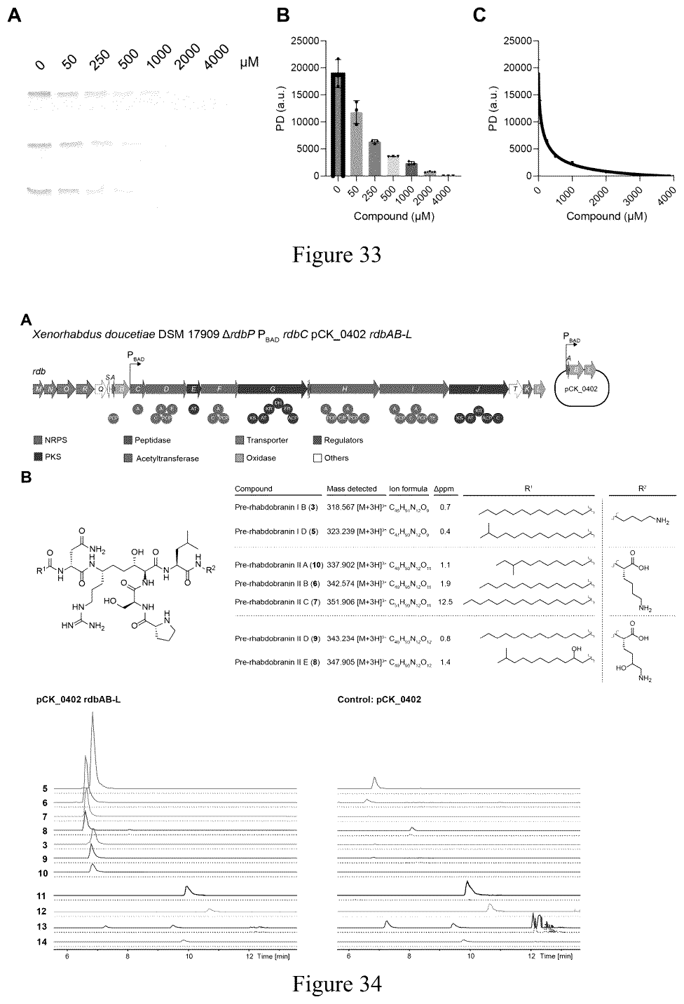

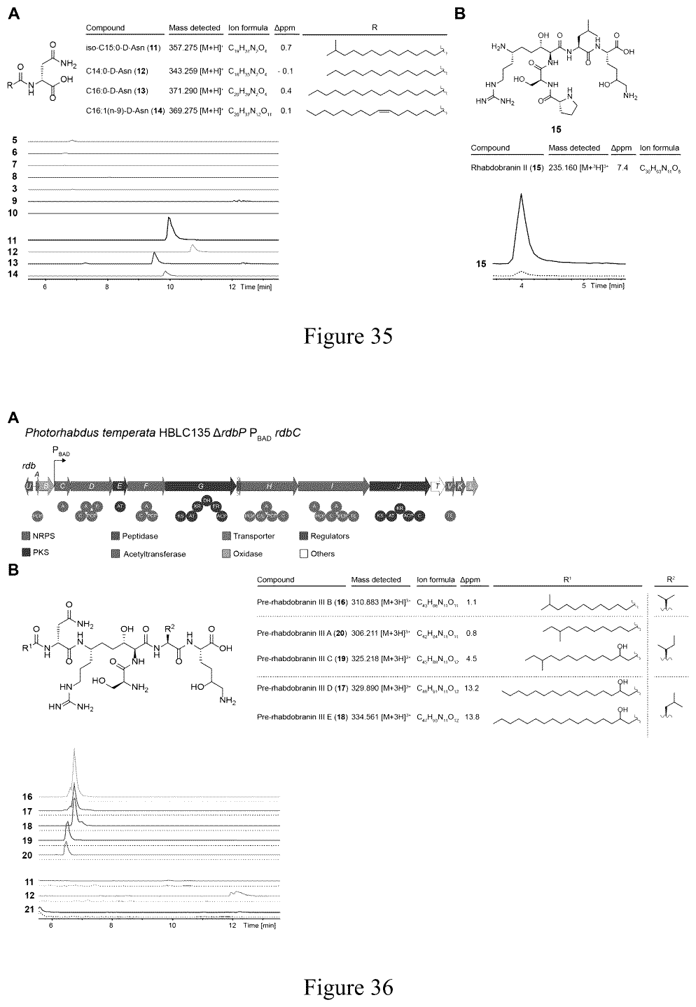

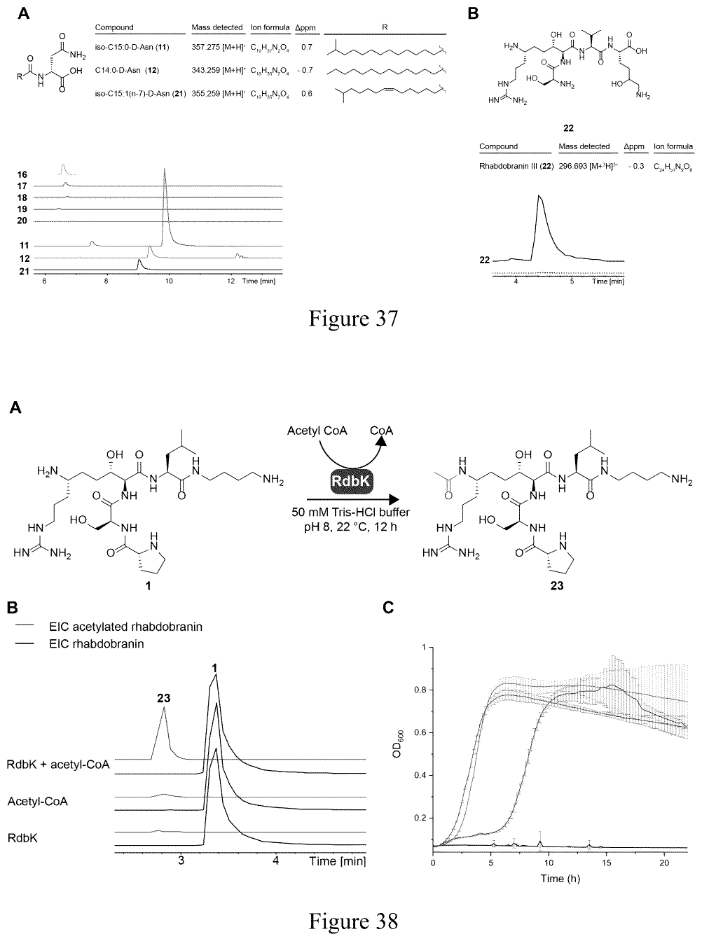

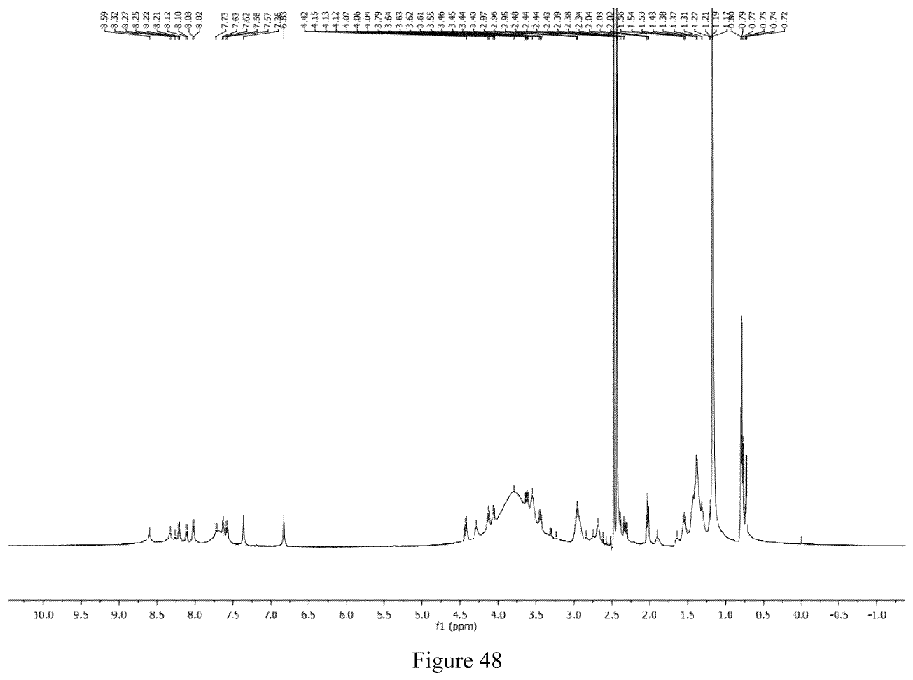

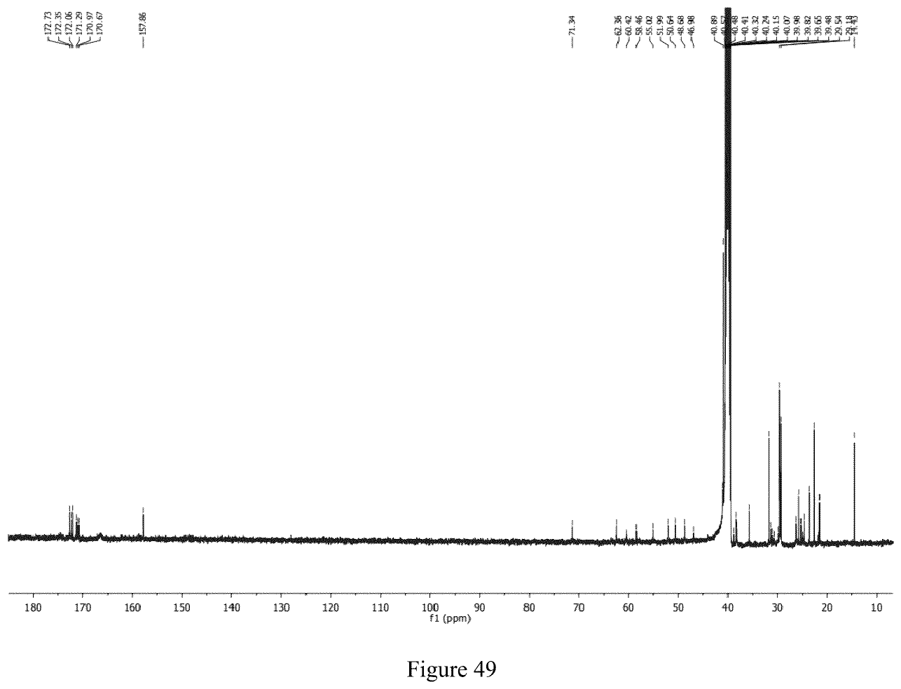

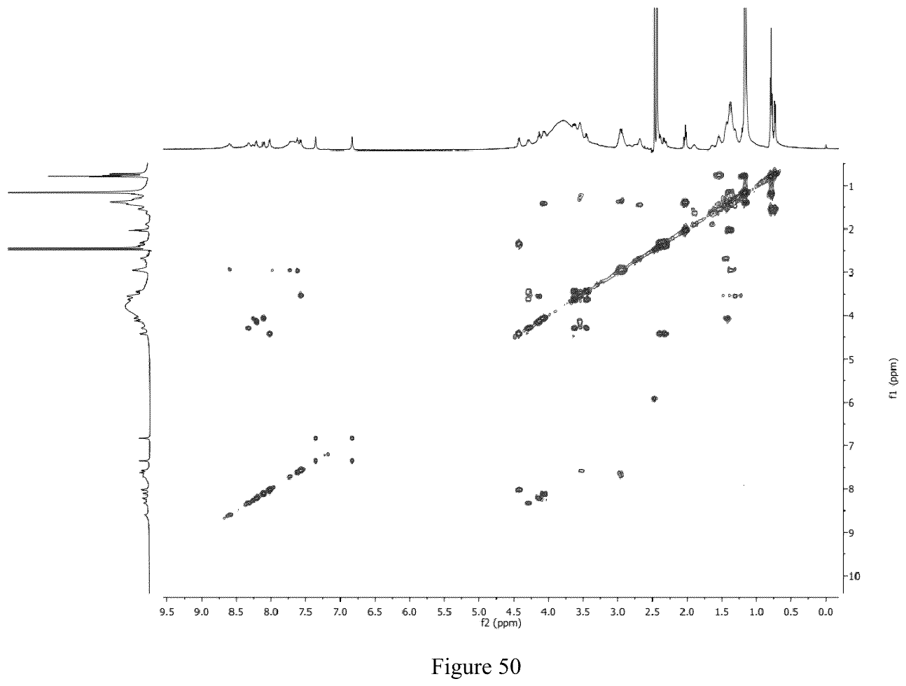

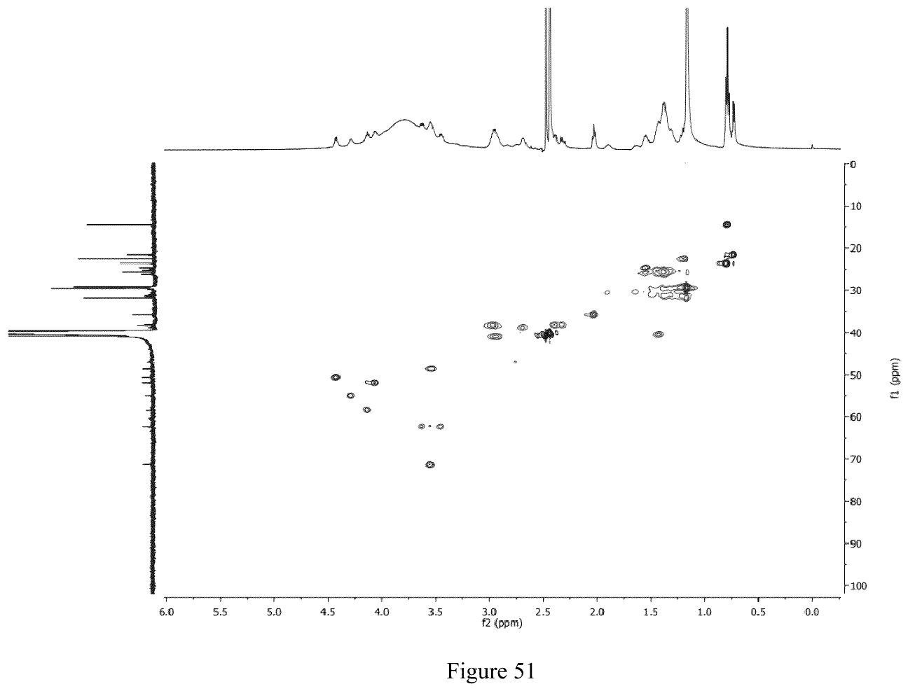

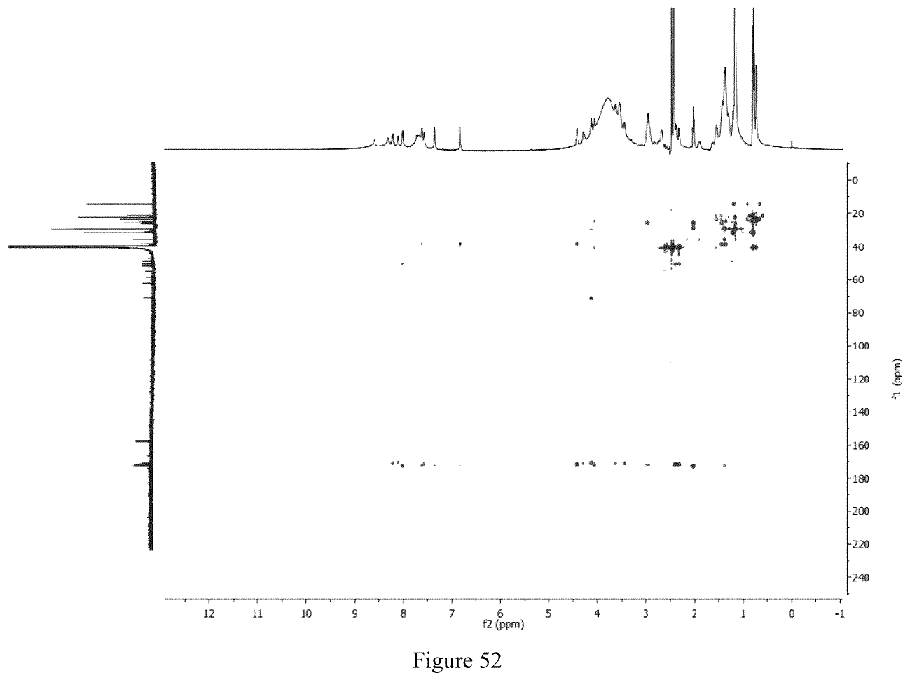

Rhabdobranin antimicrobial properties. For the verification of the predicted antimicrobial properties of rhabdobranin a disc diffusion assay was conducted, therefore an increasing concentration of either the isolated rhabdobranin I (1) or the chemically synthesized pre-rhabdobranin I B (3) (Synthesized by: Woonkee S. Jo, Group of Alan Healy, NYU Abu Dhabi; NMR structure verification: FIG. 48-52, (Supplements)) was tested against Bacillus subtilis B168, Escherichia coli MG1655, Micrococus luteus, and Saccharomyces cerevisiae CEN.PK2 (FIG. 31). In case of rhabdobranin I (1), zones of inhibition were observed for all tested strains, whereby the zones of inhibition against Micrococus luteus showed the greatest diameters, followed by the zones forming against Bacillus subtilis B168, Escherichia coli MG1655 and Saccharomyces cerevisiae CEN.PK2. In case of Bacillus subtilis B168, Escherichia coli MG1655 and Micrococus luteus a zone of inhibition was observed from the lowest applied rhabdobranin I (1) concentration, while the inhibition zones against Saccharomyces cerevisiae CEN.PK2 were observed from the second lowest applied rhabdobranin I (1) concentration. Pre-rhabdobranin I B (3) showed activity against Micrococus luteus and Bacillus subtilis B168, however, the prodrug led to the formation of inhibitory zones with a smaller diameter compared to rhabdobranin I (1), verifying that rhabdobranin I (1) showed significantly elevated antimicrobial properties compared to pre-rhabdobranin I B (3).

Rhabdobranin mediated inhibition of translation. As comparable peptidase mediated self-resistance mechanisms were previously described for the biosynthesis of compounds inhibiting the bacterial protein biosynthesis, rhabdobranin was also suggested to be either a ribosomal or an RNA-polymerase inhibitor. To verify this hypothesis, the fluorescence development of a cell-free expression of enhanced green fluorescent protein (eGFP) in presence and absence of rhabdobranin I (1) was recorded. As shown below the detected fluorescence development of eGFP over a time course of 16 hours, was significantly lowered in presents of rhabdobranin, compared to the expression with no drug molecule added. Remarkably fluorescence suppression was higher than for the ribosome inhibitor chloramphenicol, which served as positive control (FIG. 32), verifying rhabdobranin I (1) inhibiting the protein biosynthesis.

Rhabdobranin BGC type II and III derivatives. As previously shown the three rhabdobranin BGC types produce different pre-rhabdobranin derivatives and thus also different rhabdobranin variants were expected to result. Due to differences in their NRPS domain architecture, predicted by antiSMASH, the derivatives were suggested to defer by the incorporated amino acid residues. Different pre-rhabdobranin derivatives were produced by generating promoter exchange mutants and converted the prodrug molecules by in vitro conversion to characterize the resulting rhabdobranin derivatives.

BGC type II: Derivatives produced in Xenorhabdus doucetiae DSM 17909. In comparison to rhabdobranin BGC type I, type II was predicted to have an active second A domain of NRPS RdbI, annotated with an antiSMASH predicted specificity for lysine. For the production of pre-rhabdobranin a promoter exchange variant of the rdbP deletion strain with a PBAD-promoter upstream of rdbC was generated, additionally, a pCK_0402 rdbAB-L plasmid was transformed into the production strain (FIG. 34A). The induction of the promoter exchange variant, transformed with the pCK_0402 construct led to the detection of compound 3 and 5 (FIG. 34B). Both pre-rhabdobranin derivatives were also detected in Xenorhabdus budapestensis DSM 16342 ΔrdbP pCEP_kan-rdbA, suggesting putrescine to lead to the hydrolysis of the growing peptide chain from RdbI, prior the addition of the second building block. Additionally, five further derivatives were detected (FIG. 34B) (Table 5, (Supplements)). Related to the HR-MS signals, three of them (6, 7 and 10) suggested with L-lysine incorporation. However, the deviation of the detected mass and the predicted sum formula of compound 7 was high, requiring further structural elucidation. Two other derivatives (8 and 9) were suggested with a S-hydroxy L-lysine incorporation, as the building block was previously reported in the odilorhabdin biosynthesis. However, the acquired HR-MS data are insufficient to draw a conclusion about the exact location of the hydroxyl group attached to the suggested incorporated L-lysine derivative. An isotope labeling experiment (FIG. 27, (Supplements)) revealed the detected derivatives with at least one L-leucine building block incorporated. In case of compound 5 and 8 the incorporation of two L-leucine building blocks was identified. This suggested the addition of a branched acyl side chain, resulting from an L-leucine-derived isovaleryl building block leading to iso-odd fatty acids. In terms of compound 10, HR-MS data and an isotope labeling experiment, which did not verify the incorporation of L-valine, suggested the branched acyl side chain, to likely results from the incorporation of an L-isoleucine-derived 2-methylbutyryl building block. However, the L-isoleucine incorporation was not confirmed by an isotope labeling experiment. An additional option could be a propionate starter incorporation resulting in a linear odd-chain fatty acid. The position of the promoter in the production strain and the necessity to express rdbA, rdbB, and rdbL from an additional plasmid, suggested the PCP domain rdbA and the oxidase genes to be independently regulated from the rhabdobranin BGC. A promoter exchange upstream of rdbA, as in case of the BGC type I activation, did not lead to the production of the detected pre-rhabdobranin type II derivatives. The structural prediction of 11 to 14 is shown in the following section.

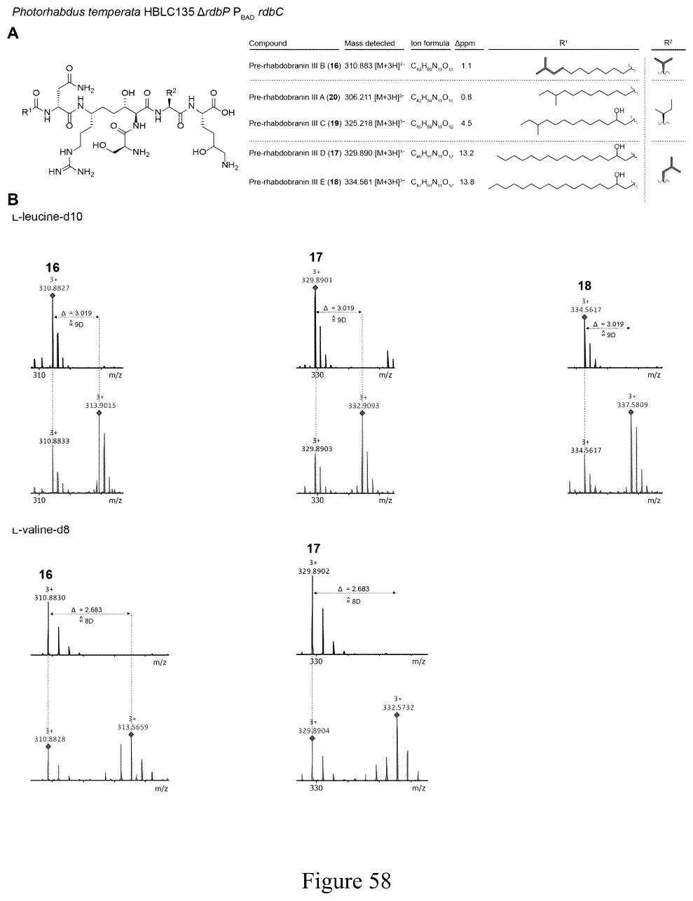

BGC type III: Derivatives produced in Photorhabdus temperate HBLC135. In comparison to rhabdobranin BGC type I, type III, like type II, was predicted to have an active second A domain of NRPS RdbI, annotated with an antiSMASH predicted specificity for lysine. Additionally, NRPS rdbH was predicted to only have one serine specific A domain instead of a proline and serine specific A domain. For the production of pre-rhabdobranin, a promoter exchange variant of the rdbP deletion strain with a PBAi-promoter upstream of rdbC was generated (FIG. 36A). The induction of the promoter exchange variant, led to the detection of pre-rhabdobranin III compound 16 to 20 (FIG. 36B). Based on the detected HR-MS masses and the predicted sum formulas (Table 5, (Supplements), the second A domain of NRPS RdbI was suggested to incorporate S-hydroxy L-lysine, a building block previously reported in the odilorhabdin biosynthesis. However, the acquired HR-MS data are insufficient to draw a conclusion about the exact location of the hydroxyl group attached to the suggested incorporated L-lysine derivative. Additional isotope labeling experiments (FIG. 58, (Supplements)) revealed the incorporation of L-leucine in case of compound 16, 17 and 18. Furthermore, L-valine was shown to be incorporated into compound 16 and 17. In case of compound 17 the predicted sum formula did not allow the incorporation of an L-valine residue, thus the compound was predicted with the incorporation of one L-leucine residues in the peptide chain as well as a propionate starter incorporation resulting in a linear odd-chain fatty acid. Considering the incorporation of L-valine detected for compound 17, it might be the case that another derivative with an L-valine residue incorporated into the peptide chain and a respectively longer linear even-chain fatty acid was co-detected at the same retention time. Notably the mass of predicted sum formulas and the detected masses of compound 17 and 18 showed a high deviation, requiring further structural elucidation (FIG. 36B). For compound 19 and 20 neither L-leucine, nor L-valine incorporation could be verified by the performed isotope labeling experiment. Due to the HR-MS masses (Table 5, (Supplements), the predicted sum formula, and the results of the isotope labeling experiments (FIG. 58, Supplements)) compound 19 and 20 was suggested with the incorporation of a branched acyl side chain resulting from a L-isoleucine derived 2-methylbutyryl building block, as well as an L-isoleucine incorporation within the peptide chain. However, an L-isoleucine isotope labeling experiments was not carried out. An additional option could be a propionate starter incorporation resulting in a linear odd-chain fatty acid. Thus, further structural elucidation of the pre-rhabdobranins III is required. The position of the promoter in the production strain, suggested the PCP domain rdbA and the oxidase genes to be independently regulated from the rhabdobranin BGC. A promoter exchange upstream of rdbA, as shown for rhabdobranin BGC type I activation, did not lead to the production of the detected pre-rhabdobranin III derivatives. The structural prediction of split-product 21 is shown in the following section.

Gcn5-Related N-Acetyltransferase conferred self-resistance. A further gene found to be encoded in all types of NRPS-PKS hybrid rhabdobranin BGCs was N-acetyltransferase rdbK. As Gcn5-Related N-Acetyltransferases were previously shown to confer a resistance of the producer strain against a specific antimicrobial compound, such as in case of odilorhabdin or amicoumacin, rdbK was a suspicious candidate conferring rhabdobranin self-resistance. Rhabdobranin was suggested to be acetylated N-terminally, as this potential acetylation position is only accessible after the peptidase mediated release of the acylated D-asparaginyl moiety, which would prevent the prodrug molecule from acetylation (FIG. 38A).

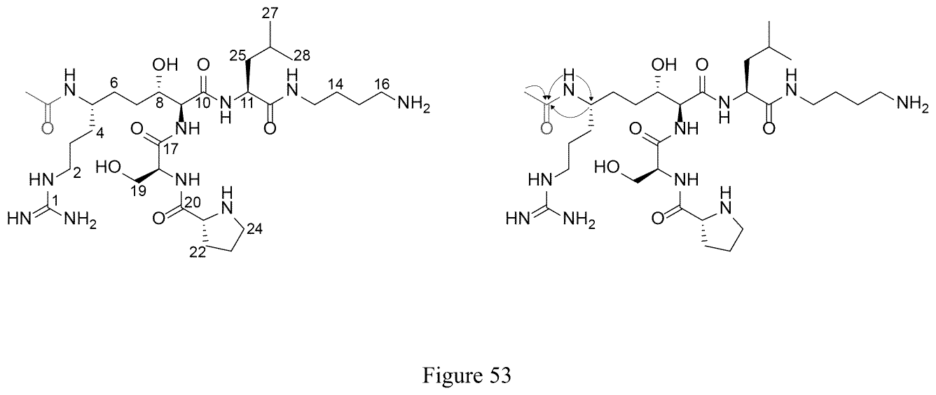

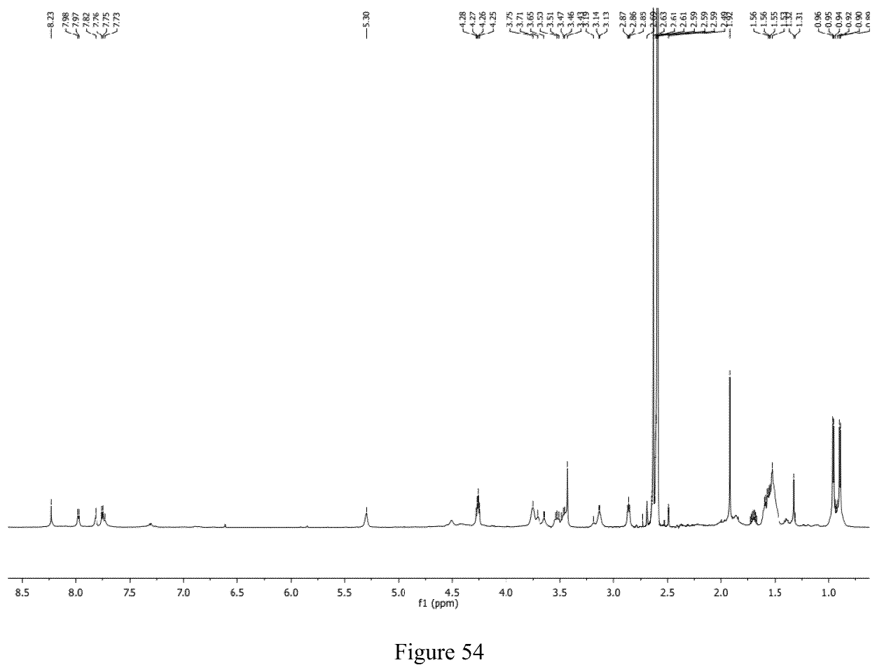

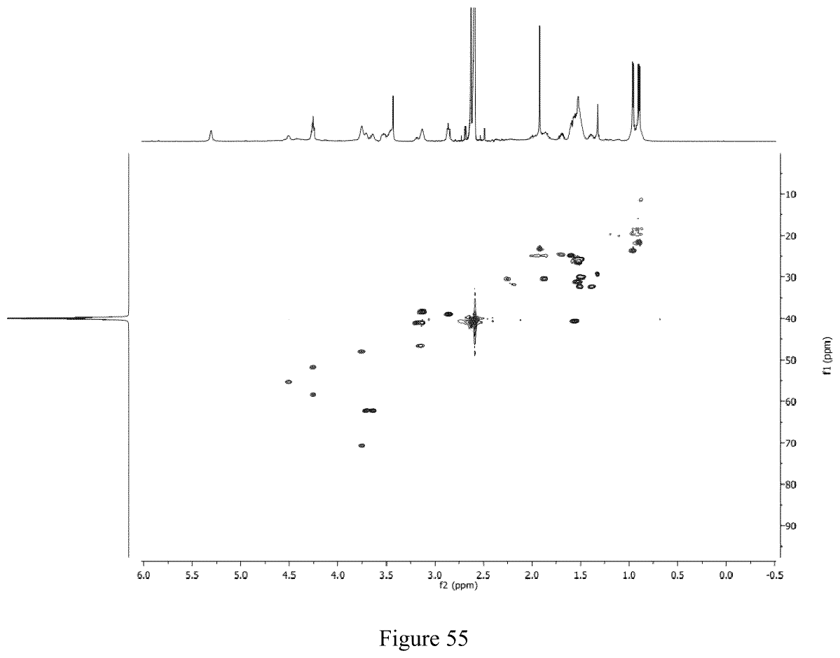

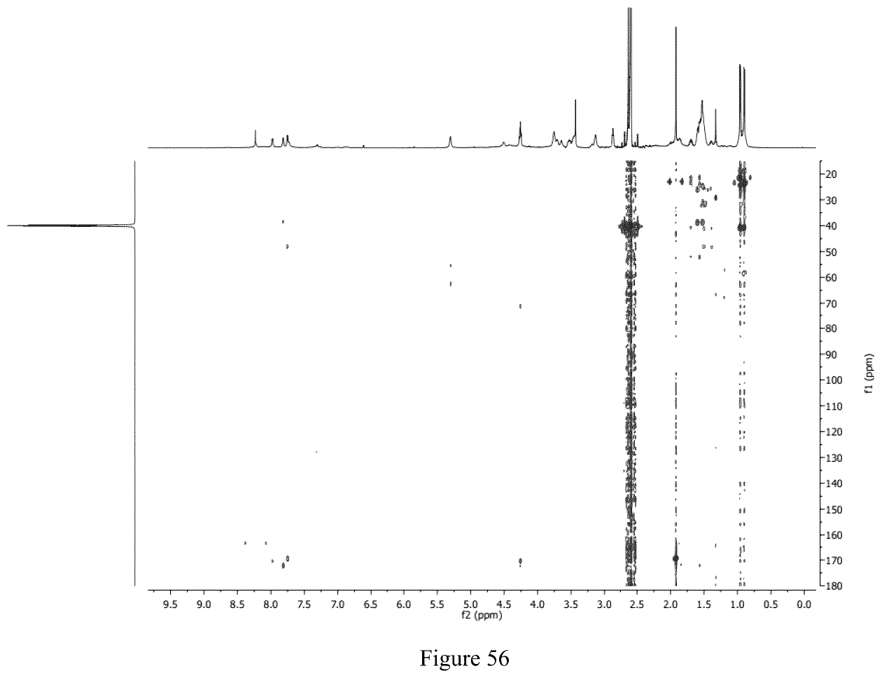

To investigate the acetylation of rhabdobranin I (1) by RdbK, the N-acetyltransferase from Xenorhabdus budapestensis DSM 16342 was heterologously expressed and isolated using metal affinity chromatography. An in vitro approach was conducted and the rhabdobranin acetylation (Compound 23) was detected in presence of RdbK and acetyl CoA by HPLC-HR-MS analysis of the reaction mix (FIG. 38B). In order to verify the suggested acetylation position, the assay was up scaled, the acetylated product was isolated and NMR analysis was carried out. The position of the acetyl group on the N-terminus of the rhabdobranin I (1) (FIG. 38A), was identified by HSQC and HMBC correlations (FIG. 53-56, (Supplements)). In a further step the growth of an acetyltransferase rdbK expressing Escherichia coli BL21(DE3), in presence and absence of rhabdobranin I (1), was compared to the one of a strain with the empty vector control (FIG. 38C). It was observed that the strain with the empty vector control did not show growth in presence of rhabdobranin, while the strain expressing rdbK showed a slightly delayed growth behavior, compared to the strains in absence of rhabdobranin I (1), entering the exponential phase after around 6 hours. This indicated the acetylation to lead to the loss of the antimicrobial properties of rhabdobranin 1(1), and hence rdbK to likely confer self-resistance.

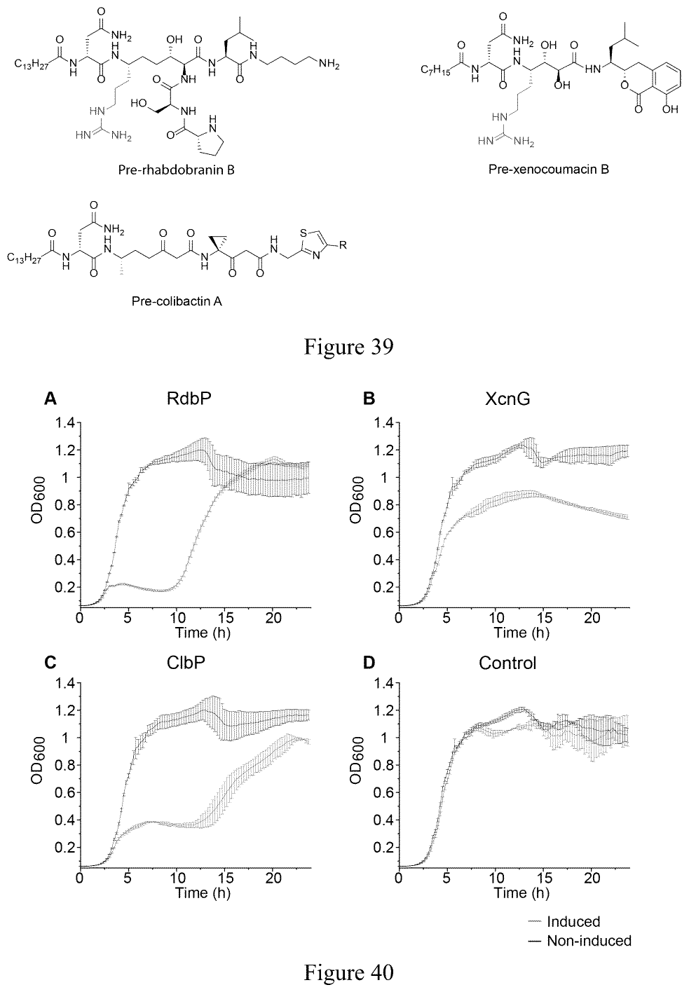

Pre-rhabdobranin activation by different self-resistance peptidases. Various peptidases encoded in BGCs were previously reported to be involved in the activation of prodrug molecules, and thus to mediate a self-resistance mechanism, as described for pre-rhabdobranin. This led to the question how specific those peptidases are, and if they are accepting a broader range of potential substrates. Here I tested if pre-rhabdobranin can also be activated by xenocoumacin peptidase XcnG or colibactin peptidase ClbP. While pre-xenocoumacin, similar to pre-rhabdobranin, was reported to have an L-arginine residue incorporated C-terminally of the acylated D-asparaginyl moiety, pre-colibactin is structurally more different, and has an L-alanine residue instead. Additionally, the length of the lipid chains varies among the compounds, while xenocoumacin B only has a C₅ chain, pre-rhabdobranin B as well as pre-colibactin A, have a C₁₄ chain (FIG. 39).

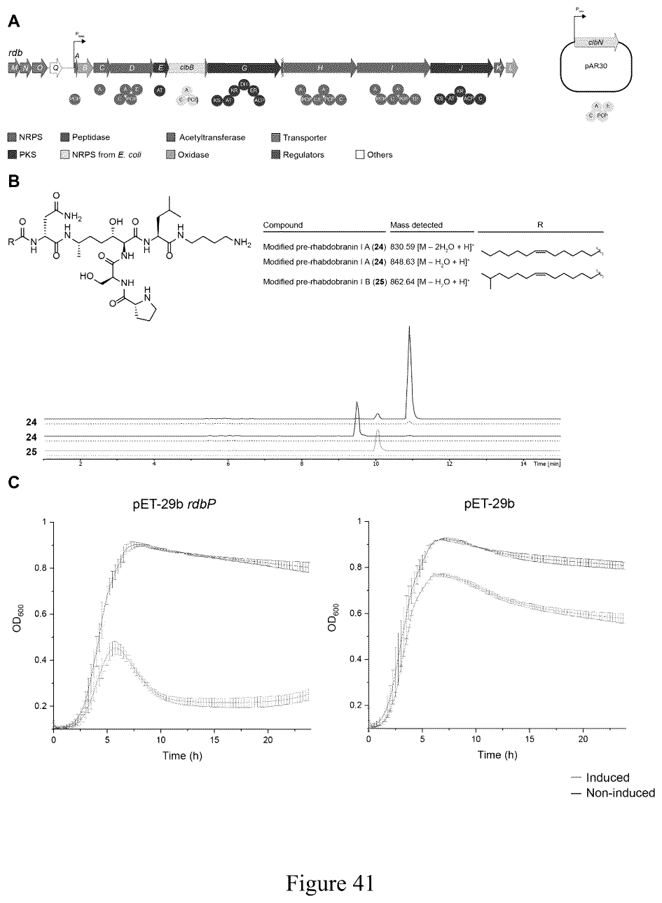

Pre-rhabdobranin modification for enhanced ClbP specificity. Colibactin producing Escherichia coli strains were found to occur enriched in mucosal tissue of patients suffering from inflammatory bowel disease and colorectal cancer, with colibactin having a mutagenic potential. As shown herein, ClbP was demonstrated to accept pre-rhabdobranin as a substrate, making it a candidate for a potential pharmaceutical application as an antibiotic specifically targeting colibactin producing Escherichia coli strains. However, applied as a medical compound, the pre-rhabdobranin activation would compete with the activation of pre-colibactin derivatives. To accelerate the conversion of pre-rhabdobranin by ClbP, and thus lowering the IC₅₀ of the compound against colibactin producing strains, an NRPS engineering approach was conducted. Here, the aim was to replace the sterically demanding L-arginine residue incorporated C-terminally of the acylated D-asparaginyl moiety by an L-alanine residue, to mimic the colibactin ClpP cleavage side, likely leading to a better coordination and thus a lowered Kₘ value of the engineered pre-rhabdobranin I derivatives. For the engineering, rdbF incorporating the L-arginine residue was replaced by a clbB, A, C and PCP-domain encoding gene fragment, incorporating the L-alanine residue in colibactin. To mediate the interaction of ClbB with PKS RdbG suggested to add the following building blocks within the rhabdobranin biosynthesis, the C-terminus of the clbB PCP-domain was replaced by the C-terminus of rdbF starting from the conserved FFxxGGxS motif in the T domain, identified as a recombination site in previous studies. Additionally, clbN catalyzing the formation of the acylated D-asparaginyl moiety in the colibactin biosynthesis was expressed from a vanillic acid inducible vector (FIG. 41A). The HPLC-MS analysis showed two engineered pre-rhabdobranin derivatives to be produced compound 24 to 25 (FIG. 41B). The structure of compounds 24 and 25 were proposed with an acylated D-asparaginyl moiety as previously reported for pre-rhabdobranin I A and I C. Notably the detected masses corresponded to respective derivatives with an eliminated hydroxyl group. The two different signals for compound 24 likely resulted from the elimination of a further hydroxyl group. This hypothesis was reinforced by the delay of the retention time of the respective detected ion, as the elimination of a hydroxyl group results in a decrease of polarity. Furthermore, it was tested whether the resulting engineered pre-rhabdobranin derivatives still have antimicrobial potential. Thus, the growth of Escherichia coli C41 ΔacrAB cells transformed with a pET-29b rdbP expression vector either 0.1 mM IPTG induced or non-induced was recorded in presence of extract containing the engineered pre-rhabdobranin derivatives (FIG. 41C). In comparison to the empty vector control, the growth of the induced RdbP expressing strain showed a reduction in growth when reaching OD₆₀₀=0.4, indicating the derivative to still have antimicrobial properties, despite the replacement of L-arginine.

### Methods: NRPS-PKS Hybrid Rhabdobranin BGC.

Strain cultivation. For the liquid cultivation of Escherichia coli as well as the respective Photorhabdus and Xenorhabdus strains, their deletion and promoter exchange variants were grown in 5 mL lysogeny broth (LB) medium (10 g/L tryptone, 5 g/L yeast extract and 5 g/L NaCl) with shaking at 37° C. for Escherichia coli and at 30° C. for Photorhabdus and Xenorhabdus. For cultivation on agar plates, 1.5% agar was added to the LB medium. For the selection of specific strains, the liquid culture as well as the agar plates were supplemented with the respective antibiotic. Production cultures were inoculated from overnight cultures (1:50), for the induction of promoter exchange variants carrying a PBAD promoter, 0.4% (w/v) L-arabinose was added to the 5 mL culture at the beginning of the 72-hour cultivation at 30° C. Production cultures with a pAR30 vector construct were induced by the addition of 50 μM vanillic acid. In case of pre-rhabdobranin derivative production cultures, 4% of Amberlite XAD-16 was added. For the isotope labeling experiments 20 μM of the respective deuterated amino acid derivative was added to the production culture (either D₈-L-valine (Sigma Aldrich) or D₁₀-L-leucine (Sigma Aldrich)).

Isolation of genomic DNA from bacterial strains. Genomic DNA of the respective strain was isolated using the Monarch Genomic DNA Purification Kit (New England Biolabs) according to the manufacturer's instructions.

Generation of promoter exchange mutants. For the on-demand activation of the rhabdobranin BGCs a promoter exchange variant carrying the arabinose-inducible PBAD promoter in front of the target gene was generated. The method is based on the conjugative transfer of a designed promoter exchange plasmid from Escherichia coli to a Photorhabdus or Xenorhabdus target strain. For the generation of the respective promoter exchange plasmid, approximately 700 bp beginning from the start codon of the target gene were amplified by PCR, and overhangs were introduced for the assembly with the pCEP_kan backbone. The pCEP_kan backbone, generated by restriction digest of the pCEP_kan plasmid by NdeI and PstI, followed by the isolation from 1.5% (w/v) agarose gel, was assembled with the generated insert using NEBuilder® HiFi DNA Assembly (New England Biolabs), according to the manufacturer's instructions. The assembled promoter exchange plasmid was transformed into Escherichia coli ST18, and verified by restriction digest with Acc65I and MiuI. Conjugation was performed as described herein.

Polymerase chain reaction. For amplification of DNA from a plasmid or genomic DNA template, polymerase chain reaction (PCR) was used. Therefore, polymerases, Q5® High Fidelity DNA polymerase (New England Biolabs), or Phusion™ High Fidelity DNA polymerase (Thermo Fisher Scientific) were used according to the manufacturer's instructions. The reaction was conducted with Lab Cycler Gradient (Sensoquest GmbH) or peqSTAR 96X Universal (VWR Peqlab) thermocyclers. The PCR product was purified from 1.5% (w/v) agarose gel.

For the verification of assembled plasmids in Escherichia coli, plasmid insertions and insertions in Photorhabdus and Xenorhabdus strains as well as deletion mutants, Phire Hot Start II DNA Polymerase (Thermo Fisher Scientific) was used. In order to perform the verification via colony PCR, a grown colony of the respective strain was picked and lysed in 20 μL of a 1:1 mix of water and DMSO under shaking (750 rpm) for 15 min, 0.5 μL of the lysate was added to a 25 μL PCR reaction mix. Further steps were performed according to the manufacturer's instructions.

Purification from agarose gel and/or PCR. For the isolation of PCR and restriction digest products from agarose gels the Monarch DNA Gel Extraction Kit (New England Biolabs) was used according to the manufacturer's instructions.

Escherichia coli transformation. Escherichia coli cells were transformed by electroporation (Dower et al. 1988). A LB culture was inoculated 1:100 from an overnight culture and grown to an OD₆₀₀=0.6. Subsequently, the cells were harvested at 4000 rpm and 4° C. for 15 min and cells were washed with 4/5 culture volume of 10% glycerol at 4000 rpm and 4° C. for 15 min. The wash step was repeated with 1/25 culture volume and 1/50 culture volume 10% glycerol. Finally, cells were resuspended in 1/500 culture volume and 50 μL aliquots were stored at −80° C. Before the electroporation, cell aliquots were thawed on ice and approx. 50-100 ng of the respective plasmid was added. Electroporation was performed using a 1 mm cuvette from either GenePulser Xcell™ (Bio-Rad Laboratories GmbH) or GenePulser® II (BioRad Laboratories GmbH) with 1250 V, 25 F, 200Ω. Subsequently, 800 μL LB medium was added and cells were incubated at 37° C. and 750 rpm for 1 h, plated on LB agar plates with respective antibiotics, and incubated overnight.

Vector verification by digest. For the verification of an assembled vector, four colonies picked from the respective Escherichia coli strain were cultured in LB overnight. On the next day, the plasmids were isolated using the Monarch® Plasmid Miniprep Kit (New England Biolabs). In the next step a restriction digest using a suitable enzyme or enzymes with restriction sites upstream and downstream of the insertion site was performed. The reaction mix was prepared according to the manufacturer's instructions (New England Biolabs), and 1% agarose gel electrophoresis was performed.

Conjugation of Photorhabdus and Xenorhabdus strains. For the conjugation of a Photorhabdus or Xenorhabdus recipient strain with an Escherichia coli donor strain carrying the plasmid to be transferred, a volume of 5 mL LB culture was inoculated from either the Escherichia coli strain (1:100) or the Photorhabdus or Xenorhabdus strain (1:50) and incubated for around 2.5 hours, to an OD₆₀₀=0.6 (Escherichia coli strains: 37° C., at 180 rpm, Photorhabdus or Xenorhabdus strain: 30° C. at 180 rpm). Afterwards, I mL of each culture was harvested, washed with 1 mL LB at 8000 rpm for 1 min twice, and resuspended in 320 μL LB. A volume of 75 μL donor and 25 μL recipient strain were mixed in a droplet on an LB agar plate and incubated overnight at 30° C. The cell mass was carefully scraped from the LB agar plate, and resuspended in 1 mL LB. A volume of 50 μL was spotted on LB agar plates containing the respective antibiotic for selection.

Generation of Photorhabdus and Xenorhabdus in-frame deletion mutants via homologous recombination. Two primer sets are required for the in-frame deletion of a single gene to amplify 1 kb long regions flanking the target gene via PCR. Overhangs to the respective other flanking fragment and the target plasmid pEB17 were introduced. The pEB17 vector backbone was generated by a restriction digest using BglII and PstI (New England Biolabs). Both, vector backbone and flanking regions were gel-purified and subsequently fused via HotFusion cloning using the NEBuilder® HiFi DNA Assembly Master Mix (New England Biolabs) according to the manufacturer's instructions. The 5 μL reaction mix was transformed into Escherichia coli ST18. The resulting pEB17 construct was verified by colony PCR with pDS132fw and pDS132rv and conjugated into the respective Photorhabdus or Xenorhabdus target strain. After incubation at 30° C. for two days, overnight cultures with and without antibiotics were prepared. The following day, −80° C. long storage cultures were prepared from cultures grown in the presence of kanamycin (25 μg/mL), while from cultures grown in plain LB medium, serial dilutions were plated on LB agar plates containing ampicillin (100 μg/mL) and 10% w/v sucrose. Subsequently, the desired deletion was confirmed by colony PCR.

Identification of rhabdobranin and derivatives using HPLC-HR-MS. The cell suspension of the respective production culture as well as references was mixed with methanol (1:1) and spun at full speed for 25 minutes. For the HPLC-HR-MS analysis of the pre-rhabdobranin derivatives, the XAD-16 of the respective production culture was incubated in 5 mL methanol for 30 minutes. The extract was dissolved with methanol (1:1) and spun at full speed for 25 minutes. Subsequently the supernatant was analyzed by HPLC-HR-ESI-UV MS, using a Dionex Ultimate 3000 LC system (Thermo Fisher), with a DAD (Impact II) or (micrOTOF II)-3000 RS UV detector (Thermo Fisher) and coupled to an Impact II or micrOTOF II electrospray ionization mass spectrometer (Bruker); Equipped with a C18 column (ACQUITY UPLC BEH, 50 mm×2.1 mm×1.7 μm, Waters); using H₂O and ACN containing 0.1% (v/v) formic acid (FA) as mobile phases. HPLC was performed at a flow rate of 0.4 ml/min with 5% acetonitrile (ACN) equilibration (β-2 min) followed by a gradient from 5-95% ACN (2-14 min, 14-15 min 95% ACN) ending with a re-equilibration step of 5% ACN (15-16 min). For internal mass calibration, 10 mM sodium formate was injected after 14 min to 15 min. The HPLC-MS analysis was set to positive mode with a mass range of m/z 100-1200 and a UV at 190-800 nm. Additionally hydrophilic interaction liquid chromatography, using an Amide column (ACQUITY UPLC BEH, 130 Å, 2.1 mm×50 mm, 1.7 m particle size, Waters) was performed. The solvents, H₂O and ACN contained 0.1% (v/v) formic acid (FA) as mobile phases. HPLC was performed at a flow rate of 0.4 ml/min with a gradient from 95-50% ACN (β-5 min, 5-7 min 5% ACN). For internal mass calibration, 10 mM sodium formate was injected within the first two min. The HPLC-MS analysis was set to positive mode with a mass range of m/z 100-1200 and a UV at 190-800 nm. For data analysis of UV-MS-chromatograms, Compass DataAnalysis 4.3 (Bruker) was used.

Large-scale in-vitro conversion of pre-rhabdobranin to rhabdobranin. For the improvement of the rhabdobranin isolation in vitro conversion of pre-rhabdobranin was performed. The heterologous production of rhabdobranin peptidase was conducted using Escherichia coli C41(DE3)_ΔacrAB pET-29b-rdbP (generated by: (Ferlemann 2022)). A ratio of 1:100 of an overnight culture was inoculated into a main culture of three times 1 L LB in 5 μL flasks, with 50 μg/mL Kₘ. The culture was incubated at 37° C. and 180 rpm until OD₆₀₀=0.6 was reached, and induced with 1 mM IPTG, followed by further cultivation for 12 h. To isolate membranes containing RdbP, cells were harvested at 10.000 rpm for 10 minutes. Further steps were conducted at 4° C. or on ice. Subsequently, the cell pellet is dissolved in 3 ml per gram wet weight of cells in French Press Buffer (20 mM Tris, pH 8, 500 mM NaCl), with the addition of 5 mM MgCl, 0.25 mg/ml lysozyme and 10 μg/ml DNase I, under stirring. Cell disruption was performed via French press (Stansted Homogeniser EP FPG12805) at 30 kpsi with three runs. For the removal of cell debris, centrifugation was performed at 14,000 rpm for 20 min (Thermo Scientific, rotor type F14-6x250y). The supernatant was then centrifuged at 40,000 rpm for 60 min in the ultracentrifuge (Thermo Scientific, rotor type 45 TI), and the resulting membrane pellet was dissolved in 4 ml per gram wet weight wash buffer (20 mM Tris-HCL pH7.8, 150 mM NaCl, 10% glycerol, 0.03% Dodecyl-β-D-maltoside). Finally, the RdbP-containing membrane was incubated at 4° C. on the roll-mixer for one hour, aliquoted into 500 μL and stored at −80° C.

For the production of pre-rhabdobranin, a 6 μL LB production culture with 50 μg/mL Kₘ, 4% of Amberlite XAD-16 in six 5 μL cultivation flask was inoculated with an overnight culture of Xenorhabdus budapestensis DSM 16342 ΔrdbP PBAD-rdbA 1:50, and induced with 0.4% arabinose. The cultivation was performed at 30° C. and 180 rpm for 72 h. The Amberlite XAD-16 was isolated and washed with one liter of deionized water, to remove hydrophilic compounds remaining from the medium. For the elution of the products from the Amberlite XAD-16, 1 μL of methanol was added and incubated at 130 rpm for 30 min, the extract was filtered using a folded filter, grade 3 m/N, dia 240 mm (MUNKTELL & FILTRAK GmbH), and methanol was evaporated. The resulting extracted products (6.93 g) were solved under ultrasonication 15 mL methanol and 15 mL of deionized water and centrifuged at 3220×g for 30 minutes. Subsequently, purification using the 1260 Infinity II system (Agilent) equipped with a 10 Prep-C18-column 250×30.0 mm (Agilent), coupled to aG6125B-LC MSD ESI-MS Agilent) (Gradient: 5% to 65% ACN to water in 40 minutes with a flow-rate of 40 mL/min, water and acetonitrile were supplemented with 0.1% of formic acid), was carried out. Under this condition, fractions from a retention time of 4 min to 11 min were taken and unified. An HPLC-HR-MS analysis of the unified fractions (1.22 g) was performed. A mass of 0.61 g of the extract was stored and used for further analysis of pre-rhabdobranin.

For the large-scale in-vitro conversion 0.610 g was solved in 305 mL IV Buffer (20 mM Tris-HCl pH 8, 150 mM NaCl), the resulting solution was centrifuged at 4000 xg for 15 minutes. The supernatant was transferred into a sterile 1 μL Erlenmeyer flask and 3.05 mL of the thawed prepared membrane was added. The in-vitro reaction mix was incubated at 22° C. and 70 rpm for 24 hours, subsequently, the reaction mix was quenched by adding 152.5 mL of acetonitrile and centrifuged at 4.000 xg for 25 min. The supernatant was lyophilized, and resolved in 6 mL of water and 1 mL methanol under ultrasonication, and centrifugation at 17.000 xg for 20 min. Subsequently, rhabdobranin was isolated, using the 1260 Infinity II system (Agilent) equipped with either an Waters XBridge BEH Amide OBD Prep column, 13 A, 5 μm, 10×250 mm (Agilent), coupled to a G6125B-LC MSD ESI-MS (Agilent) (Gradient: 90% to 61% ACN in 40 minutes at a flow rate of 3 mL/min); Fractions from a RT of 23:72 min to 26:29 min were taken, unified and lyophilized, resulting in 15 mg of rhabdobranin. NMR analysis was performed in order to verify the purity of the isolated compound.

Isolation of chemically synthesized pre-rhabdobranin. Purification of chemically synthesized pre-rhabdobranin (Synthesized by: Woonkee S. Jo, Group of Alan Healy, NYU Abu Dhabi) was performed by using the 1260 Infinity II system (Agilent) equipped with an Eclipse XDB-C18-column, 13 A, 5 μm, 10×250 mm (Agilent), coupled to a G6125B-LC MSD ESI-MS (Agilent) (Gradient: 10% to 39% ACN in 29 minutes at a flow rate of 3 mL/min); Fractions from a RT of 23:30 min to 24:30 min were taken, combined and lyophilized.

NMR analysis. ¹H and ¹³C NMR, ¹H-¹³C heteronuclear single quantum coherence (HSQC), ¹H-¹³C heteronuclear multiple bond correlation (HMBC), and ¹H-¹H correlation spectroscopy (COSY) were measured. Chemical shifts (6) were reported in parts per million (ppm) and referenced to the solvent signals. Data are reported as follows: chemical shift, multiplicity (br=broad, s=singlet, d=doublet, t=triplet, dd=doublet of doublet, m=multiplet, and ov=overlapped), and coupling constants in Hertz (Hz). All measurements were performed using a Bruker AV500 spectrometer. The identification of the acetylated rhabdobranin was carried out by using a Bruker AV700.

Disc diffusion assay for the determination of anti-microbial properties. The antimicrobial activity of rhabdobranin was evaluated by using the following indicator organisms: Bacillus subtilis B168, Escherichia coli MG1655, Micrococcus luteus, and Saccharomyces cerevisiae CEN.PK2. A 1 mL culture of each indicator organism with an OD₆₀₀=1.0 was spread on LB or in case of Saccharomyces cerevisiae CEN.PK2 on YPD agar plates (20 g/L peptone, 10 g/L yeast extract, 20 g/L dextrose and 20 g/L agar). The respective concentration of isolated pre-rhabdobranin, rhabdobranin, or derivatives were transferred onto a 3 mm sterile filter disk and dried under a clean bench. Following the disks were place on the dried agar plates, which were then incubation at 30° C. for 12 h.

Fluorescence assay for the verification of translational inhibition. For the assay, the myTXTL Cell-Free Protein Synthesis System (arbor biosciences) was used according to the manufacturer's instructions. To each reaction mix, either the respective rhabdobranin, chloramphenicol (positive control) or a comparable volume of water was added. The measurement was performed at 37° C. using a CFX Opus 96 Real-Time PCR System (BioRad).

Identification of ribosomal inhibition and rhabdobranin IC₅₀ determination. In order to visualize the impact of rhabdobranin on protein synthesis, a coupled transcription-translation cell-free system was used (Vinogradova et al. 2020). Reactions were carried out in 10 μL of the purified components from the PURExpress In Vitro Protein Synthesis Kit (E6800S/L, NEB, Ipswich, Massachusetts) with 0.15 M DNA encoding an Escherichia coli dihydrofolate reductase template, in presence of 0 μM to 4000 μM of rhabdobranin. These reactions were incubated at 37° C. for 1 h. After incubation, sample processing and fluorescent labeling were carried out as described by the Lumio Green Detection Kit (LC6090, ThermoScientific, Waltham, Massachusetts) for in vitro reactions. Finally, 10 μL of labeled samples were loaded into 20% SDS-PAGE gels and visualized under a Blue-light LED transilluminator with orange filter (Cleaver Scientific LTD, Warwickshire, UK) (Laemmli 1970). Gel quantification was performed by pixel densitometry analysis using ImageJ software.

Generation of pCK_0402 constructs. For the generation of the pCK_0402 constructs, the vector backbone was amplified by PCR using primer pCK_0402_LP_Fw and pCK_0402_LP_Rv. The corresponding insert was generated from genomic DNA of the target strain with overhangs to the respective other flanking fragment and the pCK_0402 backbone. Both, vector backbone and inserts were gel-purified and subsequently fused via HotFusion cloning using the NEBuilder® HiFi DNA Assembly Master Mix (New England Biolabs) according to the manufacturer's instructions. The 5 μL reaction mix was transformed into Escherichia coli DH10b, for amplification, as described herein. The resulting construct was isolated using the Monarch Plasmid Miniprep Kit (New England Biolabs), according to manufacturer's instructions, and heat shock transformation of the Xenorhabdus target strain was carried out.

Heat shock transformation of and Xenorhabdus strains. For the heat shock transformation of Xenorhabdus doucetiae DSM 17909 ΔrdbP pCEP_kan-rdbC with the respective pCK_0402 vector, a fresh LB culture was inoculated 1:100 from an overnight culture and grown at 30° C. and 180 rpm to an OD₆₀₀=0.6-0.8. Afterwards, 1 mL of the culture was harvested at 10000 rpm and 4° C. for 1 min. Further steps were carried out using fresh buffers and under cooling on ice. The cells were washed with 1 mL 4° C. cold XTB1 (100 μL 1 M Tris-HCl pH 6.5, 600 μL CaCl₂), 9.3 mL H₂O, filter sterilized) at 10000 rpm and 4° C. for 1 min and finally resuspended in 200 μL XTB1. Subsequently, 7 μL DMSO was added and the cells were incubated for 5 min. The addition of 7 μL of Dimethyl sulfoxide (DMSO) was repeated and approx.150 ng plasmid DNA was added after 5 min of incubation. After 30 min the heat shock was conducted, therefore the cells were incubated at 37° C. for exactly 3 min and incubated on ice for 2 min. Then 800 μL LB was added for incubation at 30° C. and 750 rpm for 1 h. Next, the entire culture volume was transferred onto an LB agar plate (25 μg/mL kanamycin and 34 μg/mL chloramphenicol) and incubated for 2 days at 30° C.

Identification of rhabdobranin and derivatives using HPLC-HR-MS. For the HPLC-HR-MS analysis of the pre-rhabdobranin derivatives, the XAD-16 of the respective production culture was incubated in 5 mL methanol for 30 minutes. The extract was dissolved with methanol (1:1) and spun at full speed for 25 minutes. The supernatant was subsequently analyzed. Further analysis were performed as described herein.

Heterologous expression, isolation and characterization of acetyl transferase XbpdK and in vitro assay conditions. For the characterization of acetyltransferase RdbK, the enzyme was produced by heterologous expression and subsequently isolated. For the expression of a SUMO-3-tagged construct of the acetyltransferase, the pET11a vector was used (Shi et al. 2022a). The vector backbone was amplified by PCR using primer pET11a for and pET_SMT3_rev, while the rbdK insert with introduced pET-11a-overhangs was amplified from gDNA. Both, the vector backbone and insert were fused using the NEBuilder HiFi DNA Assembly (New England Biolabs). The plasmid was transformed into Escherichia coli BL21 (DE3) for expression.

An overnight culture of the strain was inoculated 1:50 in 50 mL LB supplemented with 50 μg/ml ampicillin and cultivated at 37° C. and 180 rpm until OD₆₀₀=0.8 was reached. Subsequently, the culture was induced by adding 1 mM IPTG and further incubated at 22° C. and 180 rpm for 12 h. The cells were harvested at 10.000 rpm for 10 minutes, and cell lysis was performed by resuspending the pellet in 5 mL BugBuster® (primary amine-free) Extraction Reagent (NOVAGEN) with 1 μL of Benzonase® Nuclease (Merck Millipore), 14 mg of cOmplete™ EDTA-free protease inhibitor (Roche), and lysozyme (200 g/mL) (Sigma Aldrich), followed by incubation under shacking at 4° C. for 45 min. All further steps were performed at 4° C. or under cooling on ice. The cell debris was removed by centrifugation at 20,000×g for 30 min, and the supernatant was carefully poured into a Falken tube. For the isolation of the protein Ni²⁺ affinity chromatography was applied. Therefore, His SpinTrap™ Ni Sepharose™ High Performance Spin columns (Cytiva) were used according to the manufacturer's instructions. The columns were equilibrated with 600 μL of the His-Tag Binding Buffer (100 mM Tris-HCL pH8, 150 mM NaCl, 20 mM Imidazole) before the supernatant containing the protein was applied onto the column. Subsequently, the columns were washed with 600 μL of His-Tag Binding Buffer two times. Finally, the His-tagged SUMO-3-RdbK protein was eluted using 200 μL of His-Tag Elution Buffer (100 mM Tris-HCL pH8, 150 mM NaCl, 400 mM Imidazole), supplemented with 10% glycerol, and stored at −80° C. For the verification of a successful protein expression and isolation, a volume of 20 μL of samples was taken at each step and SDS-PAGE was conducted (Laemmli 1970).

For the verification of the acetylation catalyzed by RdbK, an in vitro approach was conducted. The 50 μL reaction was performed in 50 mM Tis-HCL pH8 buffer using a concentration of 10 μM RdbK, 1 mM rhabdobranin and 1 mM acetyl CoA (Acetyl coenzyme A lithium salt, Sigma-Aldrich). Additionally controls either containing RdbK or acetyl CoA were prepared. The reaction mixture and controls were then incubated at 30° C. for 12 hours. After quenching by adding 50 μL of ACN and centrifugation at full speed for 25 min, HR-MS-analysis was performed using the Impact II qTOF (Bruker) system as described in section 3.2.10.

In order to investigate the acetylation site of RdbK the reaction mix was scaled up. Thus, the concentration of rhabdobranin and acetyl CoA solved in 50 μL of 50 mM Tris-HCL pH8 was adjusted to 65 mM and 130 mM. The following steps were conducted as previously described, subsequently the acetylated rhabdobranin was isolated using the 1260 Infinity II system (Agilent) equipped with a Waters XBridge BEH Amide OBD Prep column, 13A, 5 μm, 10×250 mm (Agilent), coupled to a G6125B-LC MSD ESI-MS (Agilent) (Gradient: 90% to 61% ACN in 40 minutes at a flow rate of 3 mL/min); Fractions from a RT of 23:72 minutes to 26:29 minutes were taken and unified. The solvent of the resulting fractions was evaporated by using a lyophilizer, and the structure was elucidated using NMR as described herein.

Growth curve for the verification of rdbK conferred rhabdobranin resistance. For the verification of the rdbK conferred rhabdobranin resistance, an L-arabinose inducible pSEVA261-P_{BAD} construct expressing rdbK was generated and transformed into Escherichia coli BL21(DE3). An overnight culture of the cells containing the empty and the rdbK expressing pSEVA261-P_{BAD}, was inoculated 1:50 into 5 mL LB with 50 μg/mL kanamycin, either induced with 04% L-arabinose or non-induced and grown at 37° C. at 180 rpm for 12 hours. The assay was performed in triplicates using a transparent 96-well plate (Corning). Each well-contained 200 μL LB with 25 μg/mL kanamycin, additionally 0.4% of L-arabinose was added to the induced group. The growth of each induced and non-induced group in the presence and absence of 0.5 mM rhabdobranin was recorded using a Spark® multimode microplate reader (TECAN) at 37° C. and 200 rpm for 24 h.

Growth curve for the identification of pre-rhabdobranin specific peptidases. In order to test the specificity of xenocoumacine peptidase XcnG and colibactin peptidase ClbP for pre-rhabdobranin from Xenorhabdus budapestensis, a fraction containing pre-rhabdobranin was isolated (see section 3.2.11, large scale production) and tested against Escherichia coli strain C41(DE3)_ΔacrAB with a pET-29b-construct expressing rdbP from Xenorhabdus budapestensis DSM 16342 as a control, xcnG or cibP. An overnight culture with 50.μg/mL kanamycin of each strain was incubated at 37° C. and 180 rpm. The assay was performed in triplicates using a transparent 96-well plate (Corning). Each well contained 200 μL LB with 25 μg/ml kanamycin and 6 μL of an aqueous solution of 1 mg/mL pre-rhabdobranin containing fraction, additionally, 1 mM IPTG was added to the induced group. The growth of each induced and non-induced group was recorded using a Spark® multimode microplate reader (TECAN) at 37° C. and 200 rpm for 24 hours. The specificity of RdbP specificity for modified pre-rhabdobranin was performed as described for pre-rhabdobranin.

Mutation and replacement of genes using CRISPR/Cas12. For the incorporation of an L-alanine instead of an L-arginine residue building block into pre-rhabdobranin a markerless replacement of the rdbF gene in Xenorhabdus budapestensis DSM 16342 by a modified Escherichia coli clbB construct with a rdbF docking domain was performed. Therefore, two homologous arms (HAs, Table x) with a length of 500 bps at the start and the end of the gene of interest were combined with two CRISPR sites (31 bps distal of the PAM motif). The resulting inserts containing HAs and the CRISPR sites (HA-L and HA-R) were purchased from Twist Bioscience. Additionally, the modified clbB insert was generated by PCR. The insertion plasmid, pAR31 was assembled with all three inserts (HA-L, HA-R) by Golden Gate cloning using the BsaI-HFv2 NEBridge® Golden Gate Assembly (New England Biolabs) according to manufacturer's instructions. The resulting construct was transformed in an ST18 strain and verified via colony PCR with primer AR703 and AR704. The ST18 strain with the respective deletion plasmid was conjugated with the Xenorhabdus strain as described in section 3.2.8.

After two days a single clone was picked and cultured overnight. The culture was adjusted to an OD₆₀₀ of 0.5 in 10 ml LB containing 50 μg/ml kanamycin and cultivated at 30° C. for around 2.5 h until an OD₆₀₀ of 1 was reached. The recombination genes were induced with anhydrotetracycline (200 ng/ml) and incubated for 1 h at 25° C. Subsequently, L-arabinose (0.4%) was added to induce Cas12, the culture was incubated for 3 h at 25° C., and 50 μL was spotted onto an LB agar plate containing kanamycin (25 μg/ml) and L-arabinose (0.4%). After further two days, the clones were verified using colony PCR (As described in section 3.2.4) and selected on agar plates containing 10% (w/v) sucrose. An overnight culture was inoculated and stored at −80° C.

Generation of pAR30 constructs. For the generation of the pAR30-clbN constructs, the vector backbone (generated by Alexander Rill, Bode Group) was amplified by PCR using primer F24_Fw and pAR30-Rv (as described herein). The corresponding insert was generated from genomic DNA of the target strain with overhangs to the respective other flanking fragment and the pAR30 backbone. Both, vector backbone and inserts were gel-purified and subsequently fused via Hot Fusion cloning using the NEBuilder® HiFi DNA Assembly Master Mix (New England Biolabs) according to the manufacturer's instructions. The 5 μL reaction mix was transformed into Escherichia coli ST18, as described herein. The resulting construct was isolated using the Monarch Plasmid Miniprep Kit (New England Biolabs), according to manufacturer's instructions, and verified by restriction digest. The resulting strain was conjugated with the modified Xenorhabdus budapestensis DSM 16342 target strain.

List of generated bacterial strains, plasmids and used oligonucleotides.

TABLE 1
Used strains

| Strain | Genotype/NRPS |
|---|---|
| E. coli BL21 (DE3) | F- ompT hsdS_{B} (r_{B}-, m_{B}-) gal dcm (DE3) |
| E. coli BL21 (DE3) pET11a- | F- ompT hsdS_{B} (r_{B}-, m_{B}-) gal dcm (DE3) transformed with pET11a-SUMO3-rdbK, expression |
| SUMO3-rdbK | strain for SUMO-tagged N-acetyltransferase rdbK (XBUDV2_06665) |
| E. coli BL21 (DE3) pSEVA261- | F- ompT hsdS_{B} (r_{B}-, m_{B}-) gal dcm (DE3) transformed with pSEVA261-P_{BAD}-rdbK, expression |
| P_{BAD}-rdbK | strain for N-acetyltransferase rdbK (XBUDV2_06665) |
| E. coli C41(DE3) ΔacrAB | F- ompT hsd SB (r- mB-) gal dcm (DE3) |
| E. coli C41(DE3) ΔacrAB pET-29b | F- ompT hsd SB (r- mB-) gal dcm (DE3) transformed with pET-29b |
| E. coli C41(DE3) ΔacrAB pET- | F- ompT hsd SB (r- mB-) gal dcm (DE3) transformed with pET-29b-clbP, expression strain for |
| 29b-clbP | peptidase clbP |
| E. coli C41(DE3) ΔacrAB pET- | F- ompT hsd SB (r- mB-) gal dcm (DE3) transformed with pET-29b-rdbP, expression strain for |
| 29b-rdbP | peptidase rdbP (XBUDV2_06645) |
| E. coli C41(DE3) ΔacrAB pET- | F- ompT hsd SB (r- mB-) gal dcm (DE3) transformed with pET-29b-xcnG, expression strain for |
| 29b-xcnG | peptidase xcnG |
| E. coli ST18 | S17 λpirΔhemA |
| E. coli ST18 pAR20-clbB (ACT- | S17 λpirΔhemA transformed with pAR20-clbB(ACT-domain)-COM(rdbF) |
| domain)-COM(rdbF) | |
| E. coli ST18 pAR30-clbN | S17 λpirΔhemA transformed with pAR30-clbN |
| E. coli ST18 pCEP_kan-rdbA | S17 λpirΔhemA pCEP_kan-rdbA, Km^{R} |
| E. coli ST18 pCEP_kan-rdbA | S17 λpirΔhemA pCEP_kan-rdbA (XBUDV2_06610), Km^{R} |
| E. coli ST18 pCEP_kan-rdbC (PT) | S17 λpirΔhemA pCEP_kan-rdbC, Km^{R}, target strain: P. temperate |
| E. coli ST18 pCEP_kan-rdbC (XD) | S17 λpirΔhemA pCEP_kan-rdbC, Km^{R}, target strain: X. doucetiae |
| E. coli ST18 pEB17 ΔrdbP | S17 λpirΔhemA with pEB17 ΔrdbP construct, Km^{R}, target strain: X. budapestensis |
| E. coli ST18 pEB17-rdbP (XD) | S17 λpirΔhemA with pEB17 ΔrdbP construct, Km^{R}, target strain: X. doucetiae |
| P. temperate HBLC135 ΔrdbP | rdbP (PTHBLC135_08700) in-frame deletion mutant |
| P. temperate ΔrdbP pCEP_kan- | rdbP (PTHBLC135_08700) deletion mutant, Km^{R}, araC, araBAD promoter upstream of rdbC |
| rdbC | (PTHBLC135_08675) |
| X. budapestensis DSM 16342 | Wild type |
| X. budapestensis | Km^{R}, araC, araBAD promoter upstream of rdbA (XBUDV2_06610) |
| pCEP_kan-rdbA | |
| X budapestensis | rdbP deletion mutant, with clbB A, C, T domain substitution of rdbF (XBUDV2_06635) A, C |
| ΔrdbP clbB(ACT-domain)- | and T domain, Km^{R}, araC, araBAD promoter upstream of rdbA (XBUDV2_06610) |
| COM(rdbF) pCEP_kan-rdbA | |
| X. budapestensis ΔrdbP | rdbP (XBUDV2_06645) in-frame deletion mutant |
| X. budapestensis ΔrdbP pCEP: kan- | rdbP (XBUDV2_06645) in-frame deletion mutant Km^{R}, araC, araBAD promoter upstream of |
| rdbA | rdbA (XBUDV2_06610) |
| X. doucetiae DSM 17909 | Wild type |
| X. doucetiae ΔrdbP pCEP_kan- | rdbP (XDv3_70901) in-frame deletion mutant, Cm^{R}, Km^{R}, araC, araBAD promoter upstream of |
| rdbC pCK_0402 - rdbAB-L | rdbC (XDv3_70901) with L-arabinose inducible pCK_0402-rdbAB-L construct |

TABLE 2
Used plasmids.

| Plasmid | Genotype/NRPS |
|---|---|
| pAR30 | pBBR1 ori, oriT, araC, araBAD, Gm^{R} |
| pAR30-clbN | pBBR1 ori, oriT, araC, araBAD, Gm^{R} with clbN insert |
| pAR31 | pBBR1 ori, oriT, sacB, araC, araBAD, tetR, crRNA framework, Km^{R} |
| pAR31 clbB(ACT- | pBBR1 ori, oriT, sacB, araC, araBAD, tetR, crRNA framework, Km^{R} with insert for the clbB A, C, T |
| domain)-COM(rdbF) | domain substitution of rdbF A, C and T domain, and 500 bp up and down stream of rdbF |
| pCEP_kan | R6Kγ ori, relaxase TraI, Km^{R}, araC, araBAD promoter |
| pCEP_kan-rdbA | R6Kγ ori, relaxase TraI, Km^{R}, araC, araBAD promoter, rdbA (XBUDV2_06610) insert |
| pCEP_kan-rdbC (XD) | R6Kγ ori, relaxase TraI, Km^{R}, araC, araBAD promoter, Xenorhabdus doucetiae DSM 17909 rdbC |
| | (XDv3_70901) insert |
| pCEP_kan-rdbC (PT) | R6Kγ ori, relaxase TraI, Km^{R}, araC, araBAD promoter, rdbC (PTHBLC135_08675) Photorhabdus |
| | temperate HBLC135 insert |
| pCK_0402 | P15A ori, araC, araBAD promoter, Cm^{R} |
| pCK_0402-rdbAB-L | P15A ori, araC, araBAD promoter, Cm^{R}, with Xenorhabdus doucetiae DSM 17909 rdbA (XDv3_70908), |
| (XD) | rdbB (XDv3_70907), rdbL (XDv3_70895) insert |
| pEB17 | pDS132 based, R6K ori, relaxase TraI, Km^{R}, cipB derivative with additional BgIII site, sacB |
| pEB17-ΔrdbP (XD) | pDS132 based, R6K ori, relaxase TraI, Km^{R}, cipB derivative with additional BgIII site, sacB with 1000 bp |
| | upstream and 1000 bp downstream of Xenorhabdus doucetiae DSM 17909 rdbP (XDv3_70901) |
| pEB17-ΔrdbP | pDS132 based, R6K ori, relaxase TraI, Km^{R}, cipB derivative with additional BgIII site, sacB with 1000 bp |
| | upstream and 1000 bp downstream of of rdbP (XBUDV2_06645) |
| pET-11a-SUMO3 | ori pBR322, Amp^{R}, T7prom-his6-smt3, Ulp1 cleavage site |
| pET-11a-SUMO3-rdbK | ori pBR322, Amp^{R}, T7prom-his6-smt3, Ulp1 cleavage site with rdbK insert (XBUDV2_06665) |
| pET-29b | ori pBR322, Km^{R}, lacO, T7prom-his6 |
| pET-29b-clbP | ori pBR322, Km^{R}, lacO, T7prom-his6, with clbP insert |
| pET-29b-rdbP | ori pBR322, Km^{R}, lacO, T7prom-his6, with Xenorhadbdus budapestensis DSM 16342 rdbP |
| | (XBUDV2_06645) insert |
| pET-29b-xcnG | ori pBR322, Km^{R}, lacO, T7prom-his6, with xcnG insert |
| pSEVA261-P_{BAD} | oriV (p15A), araC, araBAD promoter, RiboJ, Km^{R}- |
| pSEVA261-P_{BAD}-rdbK | oriV (p15A), araC, araBAD promoter, RiboJ, Km^{R}, with rdbK (XBUDV2_06665) insert |

TABLE 3
Used oligonucleotides. All sequences in the following table are written 3′ to 5′ but
appear in the sequence listing in the 5′ to 3′ orientation using the SEQ ID NO as provided.

| Plasmid | Oligonucleotide | Sequence (3′→5′; overlapping ends) | Template |
|---|---|---|---|
| | AR_pSEVA_261- | CGCCAGGGTTTTCCCAGTCACGAC (SEQ ID NO: 1) | pSEVA 261 |
| | P_{BAD}_FW | | |
| | AR_pSEVA_261- | CTAGTATTTCCCCTCTTTCTC (SEQ ID NO: 2) | pSEVA 261 |
| | P_{BAD}_RV | | |
| | AR703 | AGCGGATAACAATTTCACACAGGA (SEQ ID NO: 3) | PAR31 |
| | AR704 | GTAACAAACCCGCGCGATTAG (SEQ ID NO: 4) | pAR31 |
| | JJC_Xb_clbB_COM_ | TCATGTTGATCGGGGCTGC (SEQ ID NO: 5) | X. budapestensis ΔrdbP |
| | ver_Fw | | clbB(ACT-domain)- |
| | | | COM(rdbF) pCEP kan-rdbA |
| | JJC_Xb_clbB_COM_ | AGGCCAAATGTACGGCAGATAA (SEQ ID NO: 6) | X. budapestensis ΔrdbP |
| | ver_Rv | | clbB(ACT-domain)- |
| | | | COM(rdbF) pCEP kan-rdbA |
| | JJC_Xb_d_rdbP_ver_ | TTACGTGGAACTGCGTCAGGA (SEQ ID NO: 7) | X. budapestensis ΔrdbP |
| | Fw | | |
| | JJC_Xb_d_rdbP_ver_ | ACGGCAATGGGACAAGGTTTT (SEQ ID NO: 8) | X. budapestensis ΔrdbP |
| | Rv | | |
| | JJC_Xd_d_rdbP_ver_ | GCAACAGGACAGGAGAAAGG (SEQ ID NO: 9) | X. doucetiae ΔrdbP |
| | Fw | | |
| | JJC_Xd_d_rdbP_ver_ | ATACCTGCAACACACAATTCTCC (SEQ ID NO: 10) | X doucetiae ΔrdbP |
| | Rv | | |
| | pDS132fw | GATCGATCCTCTAGAGTCGACCT (SEQ ID NO: 11) | pEB17 |
| | pDS132rv | ACATGTGGAATTGTGAGCGG (SEQ ID NO: 12) | pEB17 |
| | pET_SMT3_rev | ACCACCAATCTGTTCACGA (SEQ ID NO: 13) | pET-11a-SUMO3 |
| | pET11a_for | TAAGGATCCGGCTGCTAAC (SEQ ID NO: 14) | pET-11a-SUMO3 |
| pAR30 | JJC_Ec_pAR30- | ATACTAGAGAAAGAGGGGAAATACTAGATGATGTC | E. coli K12 |
| | clbN_Fw | GGGCAATCCGTT (SEQ ID NO: 15) | |
| PAR30 | JJC_Ec_pAR30- | AGTCGCCAGGGTTTTCCCAGTCACGACTTATAGTGT | E. coli K12 |
| | clbN_Rv | CCACAAAGTCGGCG (SEQ ID NO: 16) | |
| PAR31 | JJC_Ec_clbB_A_C_T- | GGCTACGGTCTCATAAGGAGTTGATCATGGATAAT | E. coli K12 |
| | F1_Fw | ACCTCTGGAGATTTTCC (SEQ ID NO: 17) | |
| PAR31 | JJC_Ec_clbB_A_C_T- | GCAATGATCTTAAGCGAATCCCCGCCGAGGGCGAA | E. coli K12 |
| | F1_Rv | AAAGGAGGCATTG (SEQ ID NO: 18) | |
| PAR31 | JJC_Xb_rdbF_COM_ | GATAGCAATGCCTCCTTTTTCGCCCTCGGCGGGGAT | X. budapestensis |
| | F2_Fw | TCGCTTAAGAT (SEQ ID NO: 19) | |
| PAR31 | JJC_Xb_rdbF_COM_ | GGCTACGGTCTCCGCTTCATCATGCTGAACTTCCTT | X. budapestensis |
| | F2_Rv | TTGCTTTA (SEQ ID NO: 20) | DSM 16342 |
| pCEP_kan | JJC_Pt_P_{BAD}_rdbC_ | GGGCTAACAGGAGGCTAGCAATGGAAACTGGTCAA | P. temperata |
| | Fw | CTTATTACTGA (SEQ ID NO: 21) | |
| pCEP_kan | JJC_Pt_P_{BAD}_rdbC_ | GCGCCGTTTAAACATTTAAATCTGCACACCGTTCAA | P. temperata |
| | Rv | GGCGGGTAAA (SEQ ID NO: 22) | |
| pCEP_kan | JJC_Xb_P_{BAD}_rdbA_ | TTTTTGGGCTAACAGGAGGCTAGCATAATGAATGT | X. budapestensis |
| | Fw | ATCTGAACGTAAGAAGTTAGTAT (SEQ ID NO: 48) | |
| pCEP_kan | JJC_Xb_P_{BAD}_rdbA_ | GGCGCCGTTTAAACATTTAAATCTGCATTTAGAATC | X. budapestensis |
| | Rv | ACCACCTGCTGAAAC (SEQ ID NO: 23) | |
| pCEP_kan | JJC_Xd_P_{BAD}_rdbC_ | TTTTTGGGCTAACAGGAGGCTAGCATAATGAATGT | X. doucetiae |
| | Fw | ATCTGAACGTAAGAAGTTAGTAT (SEQ ID NO: 24) | |
| pCEP_kan | JJC_Xd_PBAD_rdbC_ | GGCGCCGTTTAAACATTTAAATCTGCATTTAGAATC | X. doucetiae |
| | Rv | ACCACCTGCTGAAAC (SEQ ID NO: 25) | |
| pCK_0402 | JJC_Xd_rdbAB_pCK_ | TTGGGCTAACAGGAGGAATTCCATGAATATATCTG | X. doucetiae |
| | 0402-Fw | AGCGTAAGA (SEQ ID NO: 26) | |
| pCK_0402 | JJC_Xd_rdbAB_pCK_ | CATTATACGAGCCGATGATTAATTGTCATTATGGAT | X. doucetiae |
| | 0402-Fw | GAGTCAGTGCAGCG (SEQ ID NO: 27) | |
| pCK_0402 | JJC_Xd_rdb- | TGACTCATCCATAACTTTTGAAACCCAATAAGATA | X. doucetiae |
| | L_pCK_0402-Fw | ATAGCCA (SEQ ID NO: 28) | |
| pCK_0402 | JJC_Xd rdb- | CATTATACGAGCCGATGATTAATTGTCATTAATACG | X. doucetiae |
| | L_pCK_0402-Rv | TATAAAAACCCTGGCCG (SEQ ID NO: 29) | |
| pCK_0402 | pCK_0402_LP_Fw | TGACAATTAATCATCGGCTCGTATAATGTG (SEQ ID | |
| | | NO: 30) | |
| pCK_0402 | pCK_0402_LP_Rv | GGAATTCCTCCTGTTAGCCCAAAAAAACG (SEQ ID | |
| | | NO: 31) | |
| pEB17 | JJC_Xb_d1_ΔrdbP_ | GGATCGATCCTCTAGAGTCGACCTGCAGATGTGAT | X. budapestensis |
| | Fw | CCTTAATGCACTGGC (SEQ ID NO: 32) | |
| pEB17 | JJC_Xb_d1_ΔrdbP_ | GATTTCATGGTGAGGAAAAAGAAATTAATCATGAG | X. budapestensis |
| | Rv | TTCCGCCCTCTT (SEQ ID NO: 33) | |
| pEB17 | JJC_Xb_d2_ΔrdbP_ | GAGGGCGGAACTCATGATTAATTTCTTTTTCCTCAC | X. budapestensis |
| | Fw | CATGAAATCCAGT (SEQ ID NO: 34) | |
| pEB17 | JJC_Xb_d2_ΔrdbP_ | TGTGGAATTCCCGGGAGAGCTCAGATCCACATCCC | X. budapestensis |
| | Rv | TGAGACTTTCAGGATA (SEQ ID NO: 35) | |
| pEB17 | JJC_Xd_d1_ΔrdbP_ | AGGATCGATCCTCTAGAGTCGACCTGCAGGCCTTTT | X. doucetiae |
| | Fw | GATGTCATTCTCAAC (SEQ ID NO: 36) | |
| pEB17 | JJC_Xd_d1_ΔrdbP_ | TATTAGCTTTTACGGGACGGAACTCCTGCGGACAC | X. doucetiae |
| | Rv | GGTTGA (SEQ ID NO: 37) | |
| pEB17 | JJC_Xd_d2_ΔrdbP_ | TCAACCGTGTCCGCAGGAGTTCCGTCCCGTAAAAG | X. doucetiae |
| | Fw | CTAATA (SEQ ID NO: 38) | |
| pEB17 | JJC_Xd_d2_ΔrdbP_ | TGTGGAATTCCCGGGAGAGCTCAGATCCCATAATG | X. doucetiae |
| | Rv | GTTATGCAGGTGGC (SEQ ID NO: 39) | |
| pEB17 | pDS132fw | GATCGATCCTCTAGAGTCGACCT (SEQ ID NO: 40) | |
| pEB17 | pDS132rv | ACATGTGGAATTGTGAGCGG (SEQ ID NO: 41) | |
| pET-11a- | JJC_Xb_SUMO- | GAGGCCCATCGTGAACAGATTGGTGGTATGCCCTT | X. budapestensis |
| SUMO3 | rdbK_Fw | ATTTTCAAAAGCAATAACTGATTTT (SEQ ID NO: 42) | |
| PET-11a- | JJC_Xb_SUMO- | CGGGCTTTGTTAGCAGCCGGATCCTTATCAATTAGT | X. budapestensis |
| SUMO3 | rdbK_Rv | TGAATCAGGAGAGACCA (SEQ ID NO: 43) | |
| pSEVA | JJC_Xb_rdbK_pSEVA | TAGAGAAAGAGGGGAAATACTAGATGCCCTTATTT | X. budapestensis |
| 261 | 621_Fw | TCAAAAGCAATAACTGATTTT (SEQ ID NO: 44) | |
| pSEVA | JJC_Xb_rdbK_pSEVA | GCCAGGGTTTTCCCAGTCACGACTCAATTAGTTGAA | X. budapestensis |
| 261 | 621_Rv | TCAGGAGAGACCA (SEQ ID NO: 45) | |

TABLE 4
Used oligonucleotides for the assembly of the CRISPR/Cas9 and pSEVA-plasmids.
All sequences in the following table are written 3′ to 5′ but appear in the
sequence listing in the 5′ to 3′ orientation using the SEQ ID NO as provided.

| Inserts | Sequence (3′→5′) |
|---|---|
| JJC_Xb_clbB-rdbF(COM)_HA-L | GGCTACGGTCTCAAGATAACGTGTGGCATCAGATACAACAATCACAACGTCTAA |
| | GAACTTTAAATAATTTCTACTGTTGTAGATGAGAAGTCATTTAATAAGGCCACTG |
| | GCTCACCTTCGGGTGGGCCTTTCTGCGCAATTGTTAGGCCAATTACCAACTTGGT |
| | GTTACCTGCGGAAAATCTCAGAACATCGATTCTGCAACATCTTATTTCTCCGGTG |
| | CAATGGTTACCGAGCTTACAGTACGTTGCGCAAAATGGTGTGACCCGTTATCTG |
| | GAAGTCTCGCCTAAAAATGTGCTGAGTTATCTCACTCAACGTGCGGGGCTGCCT |
| | ATGTGTTCGCTGTGGGGGGCGGATGAAATGTTCACCTCCATTCAGGCATTAAGC |
| | ACGCAAGAGAATCGGTTGAAACAGTTTTCAGGATACTGTTATTTCCACCTTTACA |
| | GCTCAAAGCTACCTGCAATTACAGAGCCAGCGGCGATAGAACAATTGTCTCGTA |
| | TTCGGAAAAACGTCAGACAGACGATGACAGGATCACGTCTGAAGGTTGAAGAG |
| | AGTCATTCTCTCTATCAACTCACACAACAGTGGCTGGAGATTGTTGAGGCAAAC |
| | GGAAATCGCCCTCTTTGCGTAGAGAAAGTGAAATTACACACACTCTTTGATGCG |
| | GTAAGTGAGACCGTAGCC (SEQ ID NO: 46) |
| JJC_Xb_clbB-rdbF(COM)_HA-R | GGCTACGGTCTCCAAGCGTGAATATACCGGTAATGAAGTTGCGATTATTGGCAT |
| | TTCCTGTCGGTTTCCCCATAGCCCGGATTGGCAGCGTTTTTGGCAAAACTTGCTG |
| | GCAGGGCGGGAATTGGTATCGTTCTTTTCTCGCGAAGAGCTTTTGGCTATTGGCA |
| | TCCCGCAGGAGGTTATTGACCAACCCAATTATGTGCCAGCCAAAGCCGCCCTTG |
| | AAGATACTGAATGCTTTGATTATCGCTTTTTCGGCTATTCGCAACGGGAAGCACG |
| | GAAGATGGACCCACAGCTTCGGGTGCTGCATGAAGTCAGTTTCAATGCTTTCCTT |
| | GATGCAGGAATGACTCCCGGAGATGCAATGCTAAATGCAGGTGTTTTCATTGGT |
| | TCCACATTGAATCTGACCCGATTTGGGCAATTTACCGGAGCCAGTCAGGATACCT |
| | CAGAAATGTTCGATATCGGAAATTATAACGATCCGGCTTCGTTTGCGGCGCAAA |
| | TCGCTTACCGCATGAAATTGAGGATCCCACACCGCATATGCTGGATCCTTGACA |
| | GCTAGCTCAGTCCTAGGTATAATACTAGTTCGAGATTTTCAGGAGCTAAGGAAG |
| | CTAAAGTCTAAGAACTTTAAATAATTTCTACTGTTGTAGATTTCGATGCGGGCGG |
| | GGATTCGCTTAAGATCAGTCTCGAGACCGTAGCC (SEQ ID NO: 47) |

TABLE 5
HR-ESI-MS data of all compounds described in this work.

| Compound | Detected mass | Calculated mass | Δppm | Ion formula |
|---|---|---|---|---|
| Rhabdobranin I (1) | 629.4423 [M + 2H]⁺ | 315.4457 [M + 2H]²⁺ | 5.4 | C₂₈H₅₆N₁₀O₆ |
| | 315.2253 [M + 2H]²⁺ | 315.2265 [M + 2H]²⁺ | 3.7 | |
| | 210.4872 [M + 3H]³⁺ | 210.4868 [M + 3H]³⁺ | −1.9 | |
| Pre-rhabdobranin I A (2) | 317.8947 [M + 3H]³⁺ | 317.8953 [M + 3H]³⁺ | 1.9 | C₄₆H₈₉N₁₂O₉ |
| Pre-rhabdobranin I B (3) | 318.5672 [M + 3H]³⁺ | 318.5672 [M + 3H]³⁺ | 0.1 | C₄₆H₉₁N₁₂O₉ |
| Pre-rhabdobranin I C (4) | 322.5668 [M + 3H]³⁺ | 322.5672 [M + 3H]³⁺ | 1.3 | C₄₇H₉₁N₁₂O₉ |
| Pre-rhabdobranin I D (5) | 323.2386 [M + 3H]³⁺ | 323.2391 [M + 3H]³⁺ | 1.4 | C₄₇H₉₃N₁₂O₉ |
| Pre-rhabdobranin II A (10) | 337.9020 [M + 3H]³⁺ | 337.9023 [M + 3H]³⁺ | 1.1 | C₄₈H₉₃N₁₂O₁₁ |
| Pre-rhabdobranin II B (6) | 342.5736 [M + 3H]³⁺ | 342.5742 [M + 3H]³⁺ | 1.9 | C₄₉H₉₅N₁₂O₁₁ |
| Pre-rhabdobranin II C (9) | 343.2337 [M + 3H]³⁺ | 343.2340 [M + 3H]³⁺ | 0.8 | C₄₈H₉₃N₁₂O₁₂ |
| Pre-rhabdobranin II D (8) | 347.9054 [M + 3H]³⁺ | 347.9054 [M + 3H]³⁺ | 1.4 | C₄₉H₉₅N₁₂O₁₂ |
| Pre-rhabdobranin II E (7) | 351.9055 [M + 3H]³⁺ | 351.9180 [M + 3H]³⁺ | 12.5 | C₅₁H₉₉N₁₂O₁₁ |
| iso-C15:0-D-Asn (11) | 357.2745 [M + H]⁺ | 357.2748 [M + H]⁺ | 0.7 | C₁₉H₃₇N₂O₄ |
| C14:0-D-Asn (12) | 343.2592 [M + H]⁺ | 343.2591 [M + H]⁺ | −0.1 | C₁₈H₃₅N₂O₄ |
| C16:0-D-Asn (13) | 371.2903 [M + H]⁺ | 371.2904 [M + H]⁺ | 0.4 | C₂₀H₃₉N₂O₄ |
| C16:1(n-9)-D-Asn (14) | 369.2747 [M + H]⁺ | 369.2748 [M + H]⁺ | 0.1 | C₂₀H₃₇N₂O₄ |
| Rhabdobranin II (15) | 235.1597 [M + 3H^{]3} | 235.1615 [M + 3H]³ | 7.4 | C₃₀H₆₃N₁₁O₈ |
| Pre-rhabdobranin III A (20) | 306.2109 [M + 3H]³⁺ | 306.2112 [M + 3H]³⁺ | 0.8 | C₄₂H₈₄N₁₁O₁₁ |
| Pre-rhabdobranin III B (16) | 310.8827 [M + 3H]³⁺ | 310.8831 [M + 3H]³⁺ | 1.1 | C₄₃H₈₆N₁₁O₁₁ |
| Pre-rhabdobranin III C (19) | 325.2180 [M + 3H]³⁺ | 325.2225 [M + 3H]³⁺ | 4.5 | C₄₅H₈₉N₁₁O₁₂ |
| Pre-rhabdobranin III D (17) | 329.8901 [M + 3H]³⁺ | 329.8944 [M + 3H]³⁺ | 13.2 | C₄₆H₉₁N₁₁O₁₂ |
| Pre-rhabdobranin III E (18) | 334.5617 [M + 3H]³⁺ | 334.5663 [M + 3H]³⁺ | 13.8 | C₄₇H₉₃N₁₁O₁₂ |
| iso-C15:1(n-7)-D-Asn (21) | 355.2589 [M + H]⁺ | 355.2591 [M + H]⁺ | 0.6 | C₁₉H₃₅N₂O₄ |
| Rhabdobranin III (22) | 296.6926 [M + 2H]²⁺ | 296.6925 [M + 2H]²⁺ | −0.3 | C₂₄H₅₁N₉O₈ |
| Acetyl-rhabdobranin I (23) | 671.4561 [M + H]⁺ | 671.4563 [M + H]⁺ | 0.3 | C₃₀H₅₉N₁₀O₇ |
| Modified pre-rhabdobranin I A (24) | 830.59 [M − 2H₂O + H]⁺ | | | |
| | 848.63 [M − H₂O + H]⁺ | | | |
| Modified pre-rhabdobranin I B (25) | 862.64 [M − H₂O + H]⁺ | | | |

Structural elucidation of rhabdobranin and derivatives.

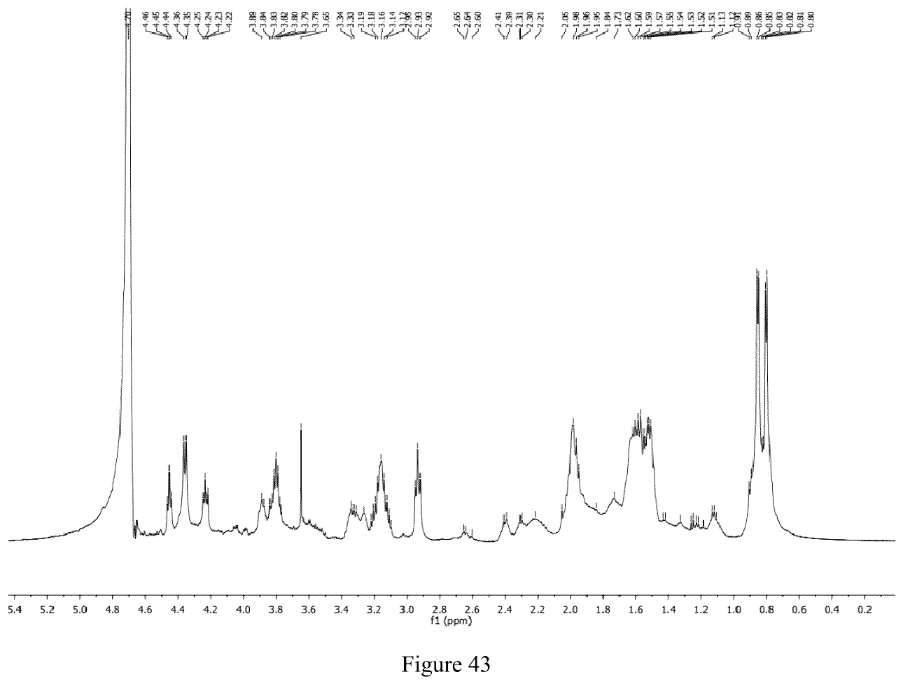

Structural elucidation of rhabdobranin I (1). See FIGS. 43 to 52.

TABLE 6
¹H (500 MHz) and ¹³C (125 MHz) NMR spectroscopic data of
rhabdobranin I (1) in DMSO-d₆ (δ in ppm and J in Hz).

Rhabdobranin

| no. | δ_{H} (mult., J) | δ_{C} |
|---|---|---|
| 1 | | 156.8 |
| 2 | 3.15, m | 40.7 |
| 3 | 1.60, m | 23.8 |
| 4 | 1.60, m | 29.0 |
| 5 | 3.26, brs | 51.3 |
| 6 | 1.73, brs | 28.0 |
| 7 | 1.52, m | 28.3 |
| 8 | 3.89, m | 70.2 |
| 9 | 4.36, d, 6.7 | 59.8 |
| 10 | | 171.4 |
| 11 | 4.23, dd, 9.6, 4.7 | 52.7 |
| 12 | | 174.2 |
| 13 | 3.12, m | 38.6 |
| | 3.19, m | |
| 14 | 1.51, m | 25.4 |
| 15 | 1.60, overlap | 23.9 |
| 16 | 2.93, d, 7.3 | 39.1 |
| 17 | | 171.6 |
| 18 | 4.45, t, 5.4 | 55.7 |
| 19 | 3.80, m | 61.4 |
| 20 | | 170.5 |
| 21 | 4.36, overlap | 58.0 |
| 22 | 2.40, m | 29.8 |
| 23 | 1.99, m | 24.1 |
| 24 | 3.33, m | 46.5 |
| 25 | 1.55, m | 39.7 |
| 26 | 1.54, m | 24.6 |
| 27 | 0.80, d, 5.6 | 20.6 |
| 28 | 0.86, d, 5.5 | 25.4 |

Structural elucidation of acetyl-rhabdobranin I (23): The position of the acetyl group was identified by the HSQC correlation from δH 3.76 (brs, —CH—) (H-5) to δC 48.1 (—CH—) (C-5) and by the HMBC correlations from δH 1.92 (s, —CH₃) 5-NHCOCH₃/7.75 (d, J=8.8 Hz, —NH) 5-NHCOCH₃/3.76 to δC 169.6 (—CO) 5-NHCOCH₃ and from δH 7.75 5-NHCOCH₃ to δC 48.1 (C-5). See FIGS. 53 to 56.

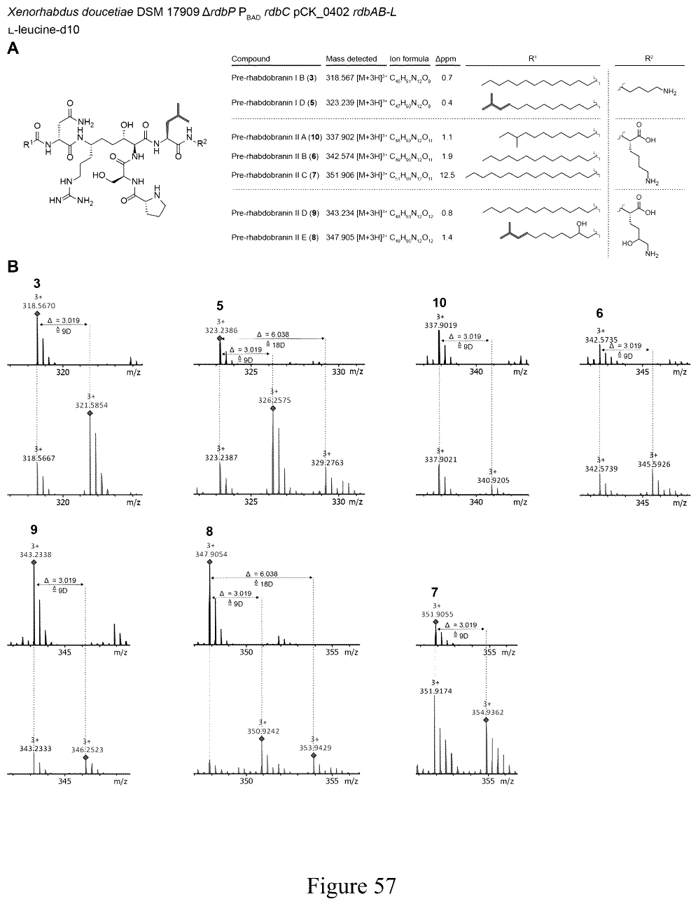

Confirmation of L-leucine and L-valine incorporation in pre-rhabdobranin II and III derivatives. See FIGS. 57 and 58.

NRPS-PKS hybrid rhabdobranin BGC. One BGC discovered by a combination of pangenomic and domain sequence similarity network approaches was the NRPS-PKS hybrid rhabdobranin BGC encoded in a Photorhabdus and various Xenorhabdus strains. AntiSMASH predictions revealed the BGC to exist in three different types, distinguishable by their NRPS A domain activity and specificity, as well as TE domain presence. An interesting feature of the clusters is an encoded peptidase, suggested to be linked to a self-resistance mechanism. As comparable peptidases were previously described in the biosynthesis of potent antimicrobial compounds, such as amicoumacin and xenocoumacin or colibactin, rhabdobranin was discussed to also possess antimicrobial properties. Even though the three BGC types as well as the prodrug molecules pre-rhabdobranin I A to D and their biosynthesis as well as the acylated D-asparaginyl split-product, were previously described, the active hydrophilic rhabdobranin molecule has never been detected nor were the predicted antimicrobial properties of the compound verified.

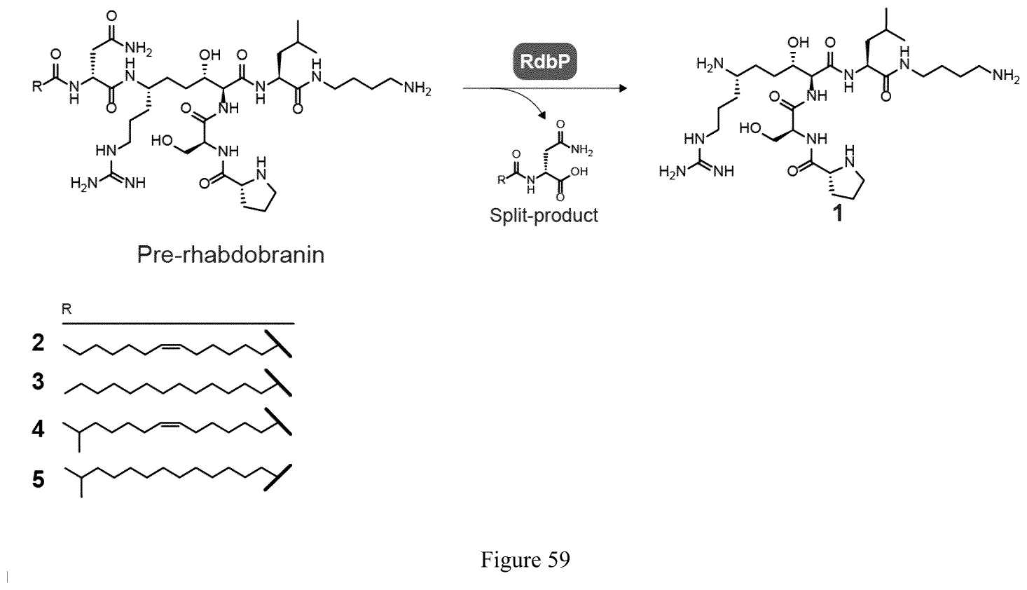

Characterization of rhabdobranin I from Xenorhabdus budapestensis DSM 16342. The hydrophilic properties of the rhabdobranin 1(1) molecule posed the major challenge for the HPLC-HR-MS detection as well as the isolation of the compound. However, the application of an adjusted gradient using a C18 column and subsequent HILIC, offering the ideal conditions for the separation of polar compounds on polar stationary phases, led to the detection of rhabdobranin 1(1). Additionally, the high rhabdobranin yield, resulting from the large-scale in vitro conversion approach, using isolate pre-rhabdobranin I A-D from Xenorhabdus budapestensis DSM 16342 and RbdP containing prepared membrane, enabled the structure elucidation of rhabdobranin 1(1) (FIG. 59).

In a next step, the antimicrobial properties of rhabdobranin I (1) were tested and compared to synthetic pre-rhabdobranin I B (3), by carrying out a disc diffusion assay, against Bacillus subtilis B168, Escherichia coli MG1655, Micrococus luteus, and Saccharomyces cerevisiae CEN.PK2. The zones of inhibition with the greatest diameter resulting from rhabdobranin I were shown in case ofMicrococus luteus and Bacillus subtilis B168, while the ones observed for Escherichia coli MG1655 and Saccharomyces cerevisiae CEN.PK2 formed at higher concentrations, with a significantly smaller diameter. In case of type I pre-rhabdobranin B (3) activity against Micrococus luteus and Bacillus subtilis B168 was observed, however the inhibitory zones had a significantly smaller diameter compared to the ones resulting from rhabdobranin 1(1). The increased resistance observed for Gram-negative Escherichia coli MG1655 in comparison to the tested Gram-positive Micrococus luteus and Bacillus subtilis B168 strain could be explained by the differences of the bacterial cell envelope architecture. While Gram-positive bacteria have a lipid bilayer membrane surrounded by a polymeric cell wall, consisting of a peptidoglycan, Gram-negative bacteria have two lipid bilayer membranes enclosing the so-called periplasm, with a high protein density and a thinner peptidoglycan cell wall in between. Thereby the outer leaflet contains specialized lipopolysaccharide and the inner one contains phospholipids. The outer membrane was reported to serve as a permeability barrier, preventing diffusion of small hydrophilic molecules, in case of Escherichia coli for molecules of up to 600 Da. This likely results in a lowered rhabdobranin I (1) uptake and hence a significantly lowered toxicity, as less molecules reach their drug target, probably located in the cytoplasm. The cell envelope architecture of Saccharomyces cerevisiae CEN.PK2 was reported to consist of one membrane, surrounded by a cell wall with two primary components, β-glucan and chitin. Whereby the small molecule permeability of the fungi and thus also of the Saccharomyces cerevisiae CEN.PK2 cell envelope can be ordered in between the Gram-negative and Gram-positive bacteria. The findings suggested that the cell wall or cell wall formation likely is not the rhabdobranin I drug target, as not only the tested bacteria, but also the Saccharomyces cerevisiae CEN.PK2 with a different cell wall composition was shown to be susceptible to rhabdobranin I (1). This indicated either translation or transcription to be inhibited by rhabdobranin I (1). Although the ribosomes of bacteria and S. cerevisiae were shown to be structurally different, with the bacterial ribosome consisting of the large 50S and the small 30S subunit and the S. cerevisiae ribosome consisting of the large 60S and the small 40S subunit, both ribosome types have a conserved core structure. Thus, it was reported that some yeast strains are susceptible for elevated concentrations of chloramphenicol; a compound investigated inhibiting translation by binding the L16 protein of the 50S subunit of bacterial ribosomes. A further potential drug target of rhabdobranin could be the transcription and thus RNA polymerase, as described for rifamycins and the fidaxomycins. As previously described for the ribosome, also the RNA polymerase consists of several subunits that are highly conserved in bacteria but not in eukaryote, a cross activity of certain inhibitors occurs when they target the highly conserved active site of the enzyme. However, the prediction of the potential drug target of rhabdobranin I (1) related to the compound's structural properties is not possible and the identification requires more experiments, testing the inhibition of potential targets. A further factor likely influencing the antimicrobial effect of rhabdobranin, are multi-drug resistance transporter. These membrane situated channels might be capable to export rhabdobranin I from the cytoplasm, leading to the reduction of the inhibitory zones, and hence the toxicity for the respective organism. An additional fact to be considered is the applied concentration of rhabdobranin I (1), which is elevated by magnitudes in comparison to the rhabdobranin probably occurring natively in the producing strain. With a yield of 15 mg per 6 μL production culture, corresponding to a concentration of 0.39 μM, the concentration of the applied solution with a concentration of 10 mM rhabdobranin 1(1) is significantly higher, potentially resulting in an increase of unspecific binding effects. In comparison to rhabdobranin I (1) the prodrug molecule pre-rhabdobranin I B (3) was shown to be less active. However, also in this case the applied concentration should be considered, potentially leading to unspecific binding. It can be concluded that rhabdobranin I (1) has shown antimicrobial properties against Gram-positive and Gram-negative bacteria as well as against Saccharomyces cerevisiae CEN.PK2 in presence of elevated concentrations, while pre-rhabdobranin B 1(3) is significantly less toxic.

In a further project subpart, the effect of rhabdobranin 1(1) in translation and transcription was tested. Therefor a cell-free expression of eGFP was carried out in presence and absence of rhabdobranin I (1), under fluorescence detection. As a positive control chloramphenicol, inhibiting translation by binding the L16 protein of the 50S subunit of bacterial ribosomes was used. It was shown that rhabdobranin 1(1) significantly reduced the expression of eGFP, even more as a comparable concentration of chloramphenicol. This indicated rhabdobranin to either inhibit transcription or translation. As the kit components originate from an Escherichia coli cell lysate, it remains uncertain if the observed effect was related to the inhibition of the bacterial RNA polymerase or the ribosome. Due to the peptidase mediated pre-rhabdobranin activation mechanism previously described for amicoumacin and xenocoumacin, inhibiting messenger RNA translation, rhabdobranin was also suggested to be a ribosome inhibitor. Hence, the IC₅₀ against the Escherichia coli ribosome was determined using a cell-free expression strategy relying on the bacteriophage T7 RNA polymerase. The single-subunit enzyme with a simple hand-like structure is one of the smallest transcription machineries, in contrast to bacterial and eukaryotic species, where the core engine, works with a variety of transcription factors to support gene expression. Thus, the ribosomal inhibition could be observed isolated from potential RNA polymerase inhibiting properties of rhabdobranin 1(1). The IC₅₀ value determined against the ribosome was 99.8 μM±17.0 μM. In comparison to chloramphenicol determined with an IC₅₀ value of 2.8 f 0.5 μM, the IC₅₀ of rhabdobranin I (1) is around 35 times higher, indicating the ribosome not to be the drug target of rhabdobranin I (1). This leads to the conclusion that rhabdobranins either require a binding partner for the inhibition of the ribosome, as shown in case of type A and B streptogramin antibiotics produced in some Streptomyces strains. Streptogramins inhibit the translation by interfering with peptide bond formation and by blocking the peptide exit tunnel in the large (50S) ribosomal subunit. Thereby type A and B act synergistically in vivo, resulting in a significantly increased toxicity. A further option would be that rhabdobranin targets the bacterial RNA polymerase. The latter hypothesis is reinforced by the eGFP cell-free expression inhibition of rhabdobranin, which was shown to be increased compared to the presence of chloramphenicol. However, more detailed analyses are required to identify the rhabdobranin drug target.

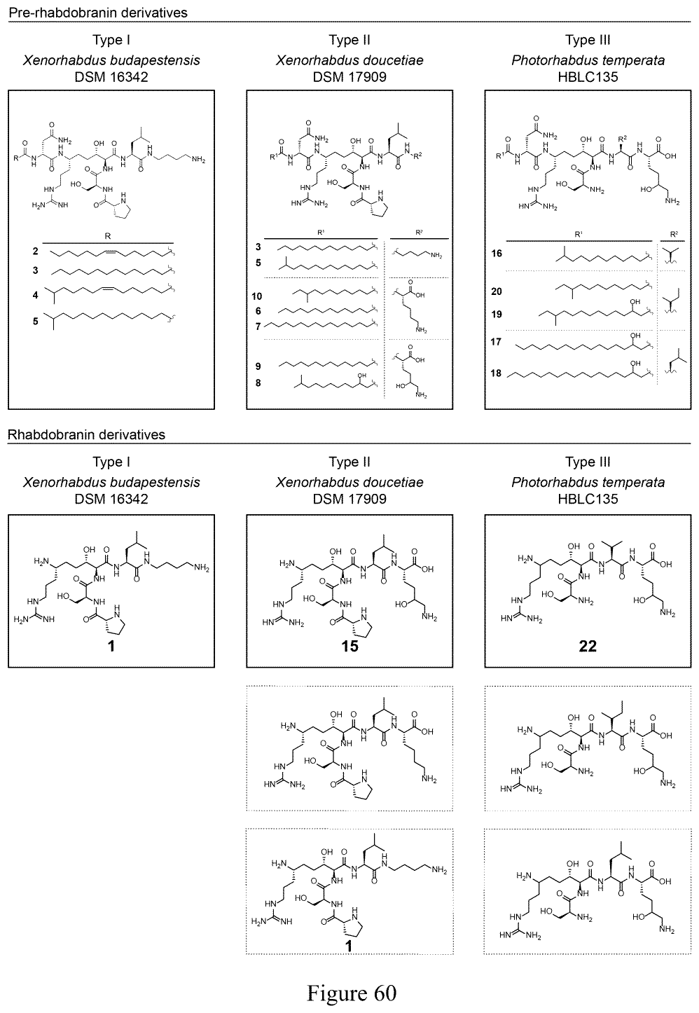

Rhabdobranin BGC type II and III derivatives. Due to differences in their NRPS domain architecture, predicted by antiSMASH, the derivatives linked to rhabdobranin BGC type I, II and III were suggested to defer by the incorporated amino acid residues. In this part of the project, I produced the different pre-rhabdobranin derivatives by generating promoter exchange mutants of Xenorhabdus doucetiae DSM 17909 and Photorhabdus temperate HBLC135, and convert the prodrug molecules by in vitro conversion to characterize the resulting rhabdobranin derivatives. The structure suggestion of the respective pre-rhabdobranin molecules thereby relied on HR-MS data and the predicted sum formula, as well as on isotope labeling experiments (FIG. 60).

The structural differences of type I, II and III pre-rhabdobranin derivatives (FIG. 60) was found to be influenced by four factors: The first factor was the added acyl side chain, which differed for each tested bacterial strain. In case of 10, 19 and 20, L-isoleucine was suggested to be incorporated as 2-methylbutyryl building block into the acyl side chain. However, the assumption resulted related to the HR-MS masses and the verification that neither L-leucine, nor L-valine was found to be incorporated. An additional option could be a propionate starter incorporation resulting in a linear odd-chain fatty acid. In terms of 16 it should be mentioned that the position of the L-leucine and L-valine still has to be verified, as the assumption relied on an isotope labeling experiment and the compound did not show fragmentation under the applied MS conditions, the results did not allow an accurate localization of incorporation. In addition, it should be mentioned that derivative 17 was predicted with the incorporation of one L-leucine residues in the peptide chain as well as a propionate starter incorporation resulting in a linear odd-chain fatty acid. Considering the incorporation of L-valine detected for compound 17, it might be the case that another derivative with an L-valine residue incorporated into the peptide chain and a respectively longer linear even-chain fatty acid was co-detected at the same retention time.

A further factor is the first RdbI A domain, found to incorporate L-leucine in case of type I and II pre-rhabdobranin. However, type III pre-rhabdobranins were shown with either L-valine, L-leucine or L-isoleucine incorporated (FIG. 60). Although, L-leucine is sterically more demanding as L-valine, the three amino acids are chemically comparable. Notably the incorporation of either L-valine or L-leucine recognized by one A domain was already reported in the biosynthesis of GameXPeptide in Xenorhabdus and Photorhabdus. Furthermore, the recognition of all three amino acids by one NRPS A domain was also already shown in the fungal cyclodepsipeptide biosynthesis. It should be mentioned that the prediction of L-isoleucine incorporation relied on HR-MS data and the L-valine as well as L-leucine isotope labeling experiments, which showed that neither L-valine, nor L-leucine was found to be incorporated into the respective derivatives.

The second RdbI A domain was also shown to have a significant influence on the diversity of the pre-rhabdobranin derivatives. While the type I second RdbI A domain was shown to be inactive, and the product release was reported to be mediated by putrescine, the type II and III domains were shown to be active (FIG. 60). Although type I derivatives 3 and 5 with a C-terminal putrescine were also found to be produced in the Xenorhabdus doucetiae DSM 17909, which could either be linked to the kinetic nature of the NRPS system, allowing the hydrolysis of the intermediate from the PCP domain by putrescine instead of the following addition of L-lysine derivatives, or the increased production titer caused by the promoter exchange. However, the domain was also shown to incorporate L-lysine (6, 7, 10) as well as δ-hydroxy L-lysine (8, 9). Notably, the domain encoded in the type III BGC was identified to exclusively incorporate δ-hydroxy L-lysine. The incorporation of δ-hydroxy L-lysine was previously described to occur in the biosynthesis of odilorhabdins in Xenorhabdus and Photorhabdus strains. In this case the A domains did also not discriminate between δ-hydroxy L-lysine and L-lysine. The production of δ-hydroxy L-lysine is catalyzed by Fe(II)/α-ketoglutarate-dependent δ-hydroxylases, which are encoded within the odilorhabdin BGC. Remarkably, neither the rhabdobranin BGCs, nor the rest of the genome of the respective strains was found to encode a homologue enzyme. Hence the enzyme catalyzing this building block in Xenorhabdus doucetiae DSM 17909 and Photorhabdus temperate HBLC135 is likely not related to the hydroxylases identified in the odilorhabdin BGC, which might result in altered hydroxylation positions of the L-lysine substrate.

In contrast to type I and II RdbH, the type III RdbH NRPS domain architecture results in the incorporation of L-serine instead of a dipeptide consisting of L-serine and L-proline (FIG. 60).

The promoter exchange for the BGC activation of type II and III pre-rhabdobranin was positioned upstream of rdbC, other than reported the exchange upstream of rdbA did not lead to the detection of the masses which were previously reported to be related to the corresponding prodrug molecules. Furthermore, the masses detected in this work deferred from the previously reported HR-MS masses. The successful activation of the BGC by the promoter exchange located upstream of rdbC, indicated the independent regulation of the BGC genes encoded downstream of rdbC and the PCP domain rdbA as well as the oxidase rdbB. Thus, the RdbA/B catalyzed building block might be incorporated in further biosynthetic pathway, resulting in a crosstalk, as previously shown for other NRPS systems.

The in vitro conversion of the pre-rhabdobranin containing extract resulted in the detection of one type II and III rhabdobranin derivative (15 and 22) (FIG. 60). Type I rhabdobranin as well as the derivative resulting from pre-rhabdobranin derivative 6, 7, 10, and in case of type III the derivatives resulting from 19 and 20 as well as 17 and 18 were not detected. As the rhabdobranin II variants not detected have a higher net positive charge in comparison to the δ-hydroxy L-lysine incorporated derivative (15), these derivatives might have been electrostatically associated to the anionic membrane, and thus likely were removed from the analyzed supernatant, as previously shown for other highly positively charged peptides. In case of the type III derivatives the L-leucine and L-isoleucine residue likely resulted in an increased hydrophobicity and thus the association to the RdbP peptidase containing membrane. The same might also apply for the acylated D-asparaginyl split-products, which were also not detectable after the in vitro conversion of pre-rhabdobranin II and III derivatives. It should be considered that the concentration of the rhabdobranin type II and III derivatives was significantly lower compared to the large-scale in vitro converted type I rhabdobranin, in which a membrane surface saturated by product molecules might have been reached.

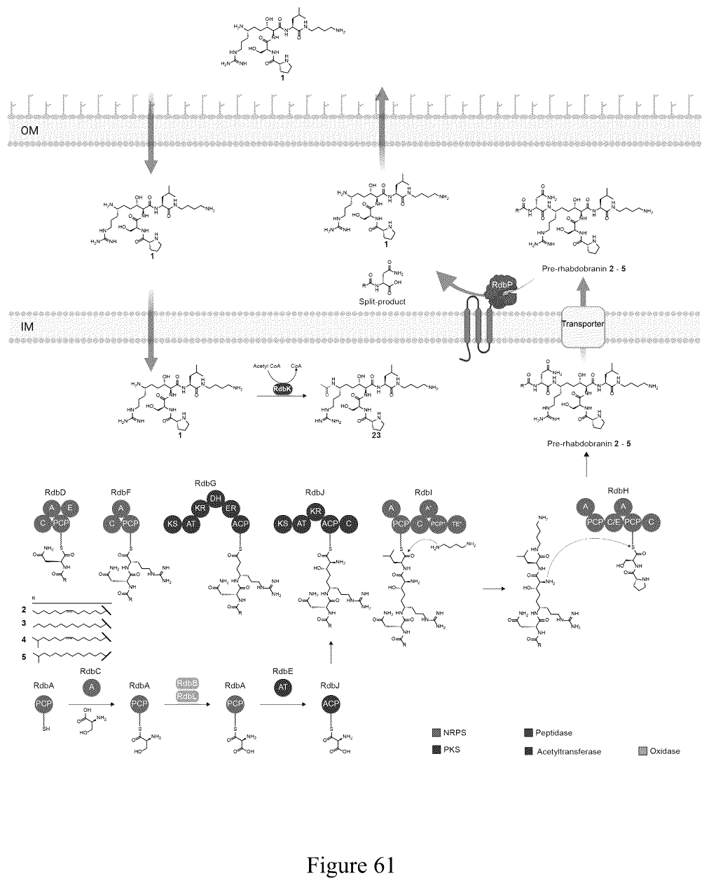

Gcn5-Related N-Acetyltransferase conferred self-resistance. The family of Gcn5-Related N-Acetyltransferases was previously identified to mediate resistance of the producer strain against a specific antimicrobial compound, such as in case of odilorhabdin or amicoumacin. Likewise, the rhabdobranin BGC encoded Gcn5-Related N-acetyltransferase rdbK was suggested to confer self-resistance. In an in vitro approach, RdbK was shown to catalyze the N-terminal rhabdobranin acetylation. Additionally, Escherichia coli strains expressing RdbK showed resistance in presence of rhabdobranin I (1). After the production of the pre-rhabdobranin derivatives in the cytoplasm, the compounds are suggested to be exported into the periplasm under the contribution of an unknown transporter. In a next step, the peptidase RdbP likely situated in the inner membrane of the producing strain, activates rhabdobranin. While the majority of the compound molecules likely transpass the outer membrane, some molecules might renter the cytoplasm. To prevent rhabdobranin from targeting the producer strain the Gcn5-Related N-Acetyltransferase confers resistance (FIG. 61). The identified acetylation position is only accessible after the peptidase mediated release of the acylated D-asparaginyl moiety, which prevents the prodrug molecule from acetylation. All rhabdobranin BGC types were shown to encode homologue Gcn5-Related N-Acetyltransferases, suggesting also type II and type III rhabdobranin derivatives to be acetylated at the same position. Furthermore, the determined data revealed that the N-terminus likely is highly important for the coordination of the active rhabdobranin molecule in its respective drug target. Therefore, it should be considered that N-acetyltransferases with a broad range of potential substrate could confer resistance against rhabdobranin, and thus limit the potential application of the compound as an antimicrobial therapeutic, as previously shown for other drug molecules.

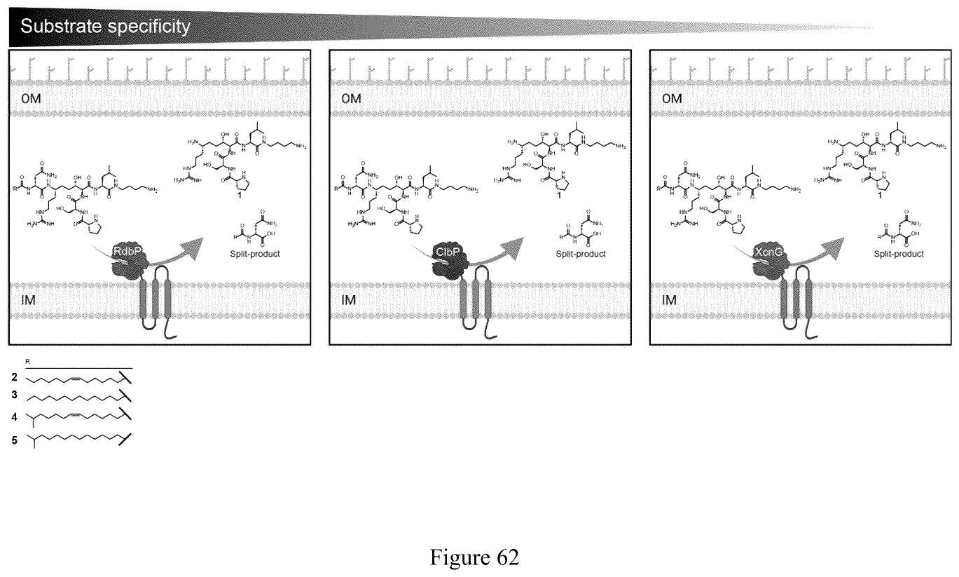

Pre-rhabdobranin activation by different self-resistance peptidases. Peptidase mediated self-resistance mechanisms were previously reported to be involved in the activation of prodrug molecules such as xenocoumacin peptidase XcnG or colibactin peptidase ClbP. Those peptidases were shown to always cleave prodrug molecules with an acylated D-asparaginyl moiety, as also described for RdbP. This led to the hypothesis that those peptidases are likely accepting a wide range of diverse substrates, equipped with the acylated D-asparaginyl feature. The growth experiment showed that pre-rhabdobranin I A, B, C and D (2, 3, 4 and 5) were activated by ClbP and XcnG (FIG. 62).

Despite the structural difference of pre-colibactin A, the native substrate of ClbP, and pre-rhabdobranins I, ClpP was identified to accept pre-rhabdobranin B as substrate, even though pre-rhabdobranins I have a sterically demanding L-arginine residue N-terminally of the acylated D-asparaginyl moiety, while pre-colibactin has an L-alanine residue at this position. The results also indicated XcnG to likely be less specific for pre-rhabdobranins I (FIG. 62), which might be linked to the fatty acid chain length differences of the native substrate xenocoumacin B, which only has a C₈ chain, while pre-rhabdobranin I B as well as pre-colibactin A, have a C₁₄ chain. Notably, after a time course of around 10 hours the growth rate of RdbP and ClbP expressing strains recovered, which might either be linked to the mutation of the vector constructs, or the lowering concentration of pre-rhabdobranins I in the growth culture. To verify the mutation a sequencing of the respective vector construct after the experiment has to be conducted. Although the used Escherichia coli C41(DE3) strains are specifically designed for the expression of membrane proteins, additionally Escherichia coli strain should be tested for the expression. The discovery of ClbP activating pre-rhabdobranin is very promising for the application of the compound as the first antibiotic specifically targeting Colibactin producing Escherichia coli strains. Which were suggested to be linked to the development of inflammatory bowel disease and colorectal cancer.

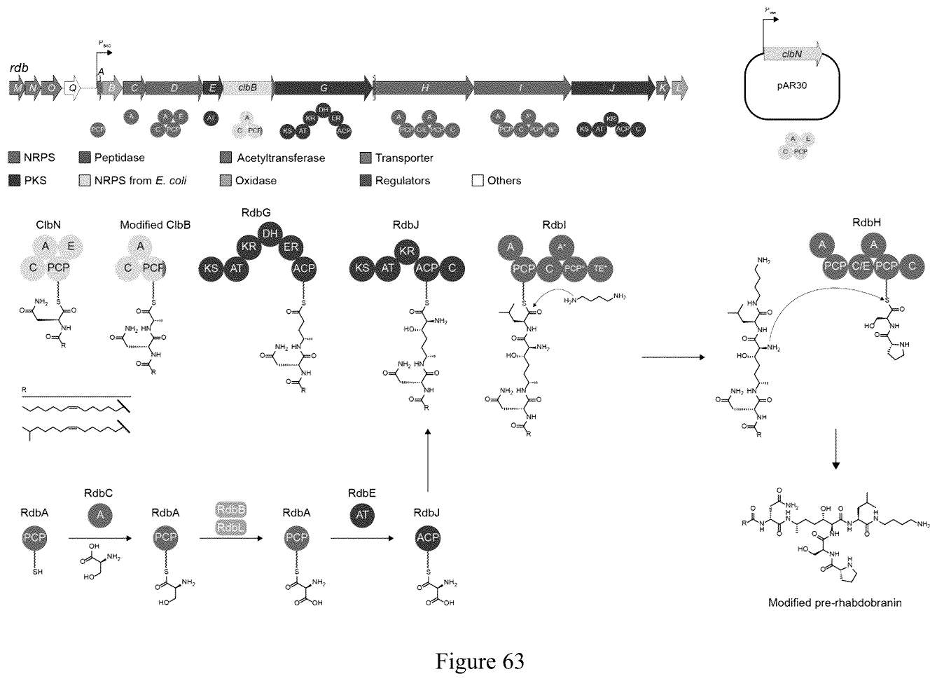

Pre-rhabdobranin modification for enhanced ClbP specificity. The discovery of ClbP to have a specificity for pre-rhabdobranin makes the compound an interesting candidate to be the first antibiotic specifically targeting colibactin producing Escherichia coli strains occurring enriched in mucosal tissue of patients suffering from inflammatory bowel disease and colorectal cancer. However, as the applied pre-rhabdobranin would have to compete with pre-colibactin derivatives, an NRPS engineering approach, to lower the IC₅₀ of the compound against colibactin producing strains, was conducted. It was aimed to replace the sterically demanding L-arginine residue incorporated C-terminally of the acylated D-asparaginyl moiety by an L-alanine residue, to mimic the colibactin CIpP cleavage side, likely leading to a better coordination and thus a lowered Kₘ value of the engineered pre-rhabdobranin derivative. The approach focused a genomic replacement, rdbF incorporating the L-arginine residue was replaced by a clbB, A, C and PCP-domain encoding gene fragment, incorporating the L-alanine residue in colibactin. To mediate the interaction of ClbB with PKS RdbG suggested to add the following building blocks within the rhabdobranin biosynthesis, the C-terminus of the clbB PCP-domain was replaced by the C-terminus of rdbF starting from the conserved FFxxGGxS motif in the T domain (FIG. 63), identified as a recombination site in previous studies. For the catalysis of the acylated D-asparaginyl moiety a clbN expressing construct was transformed into the engineered strain, as RdbD, catalyzing the moiety in the producer strain, was not expected to interact with ClbB for the addition of the L-alanine building block (FIG. 63). The HPLC-MS analysis of the induced production culture of the modified strain showed the formation of two different modified type I pre-rhabdobranin derivatives, allowing the prediction of the modified biosynthetic mechanism. For a more detailed prediction, HR-MS data have to be acquired. Furthermore, it was shown that the derivatives can still be activated by RdbP. The acquired data indicated that the L-arginine residue might not be essential for the antimicrobial properties of rhabdobranin. An additional advantage of the structural modification of pre-rhabdobranin could be a reduced specificity of the Gcn5-Related N-Acetyltransferases for the active modified rhabdobranin molecule, ideally reducing a mediated resistant effect.

Although the present disclosure has been described with respect to one or more particular embodiments and/or examples, it will be understood that other embodiments and/or examples of the present disclosure may be made without departing from the scope of the present disclosure.

1. A method for selectively killing at least a portion of an E. coli population, wherein the population of E. coli expresses a ClbP, comprising contacting the E. coli population with a prodrug antibiotic, wherein the prodrug antibiotic is cleaved in the E. coli and a therapeutically effective amount of an antibiotic is formed and at least a portion of the E. coli population is killed, wherein the prodrug antibiotic is chosen from 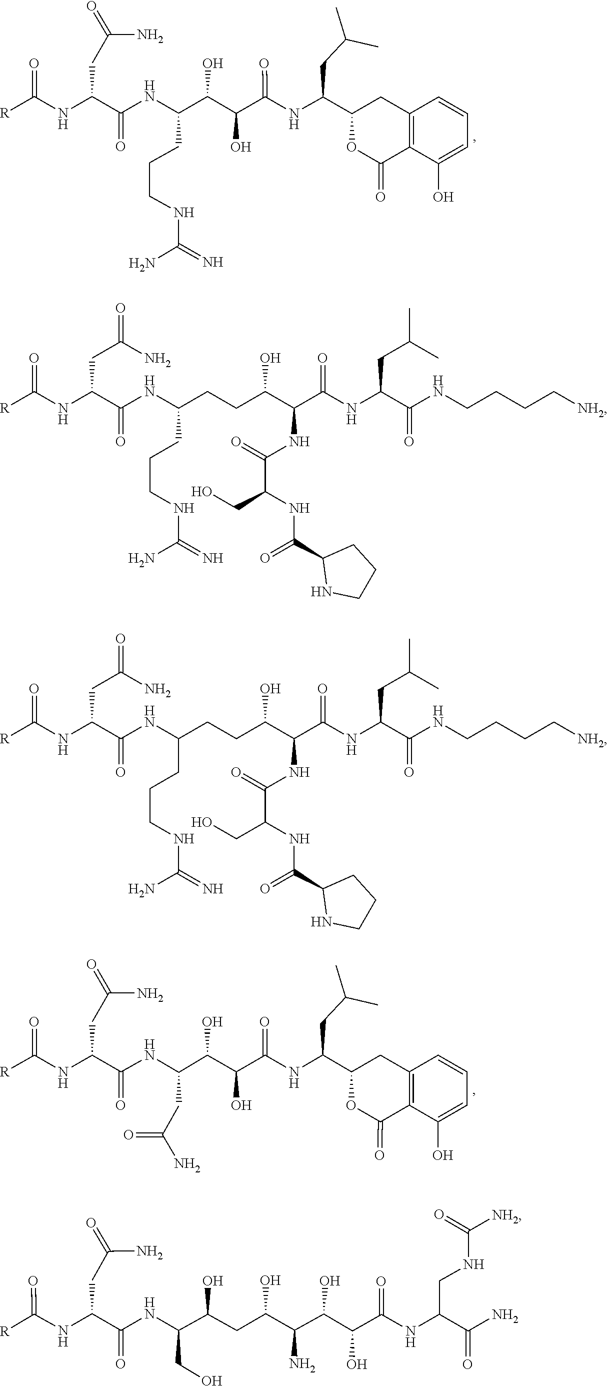 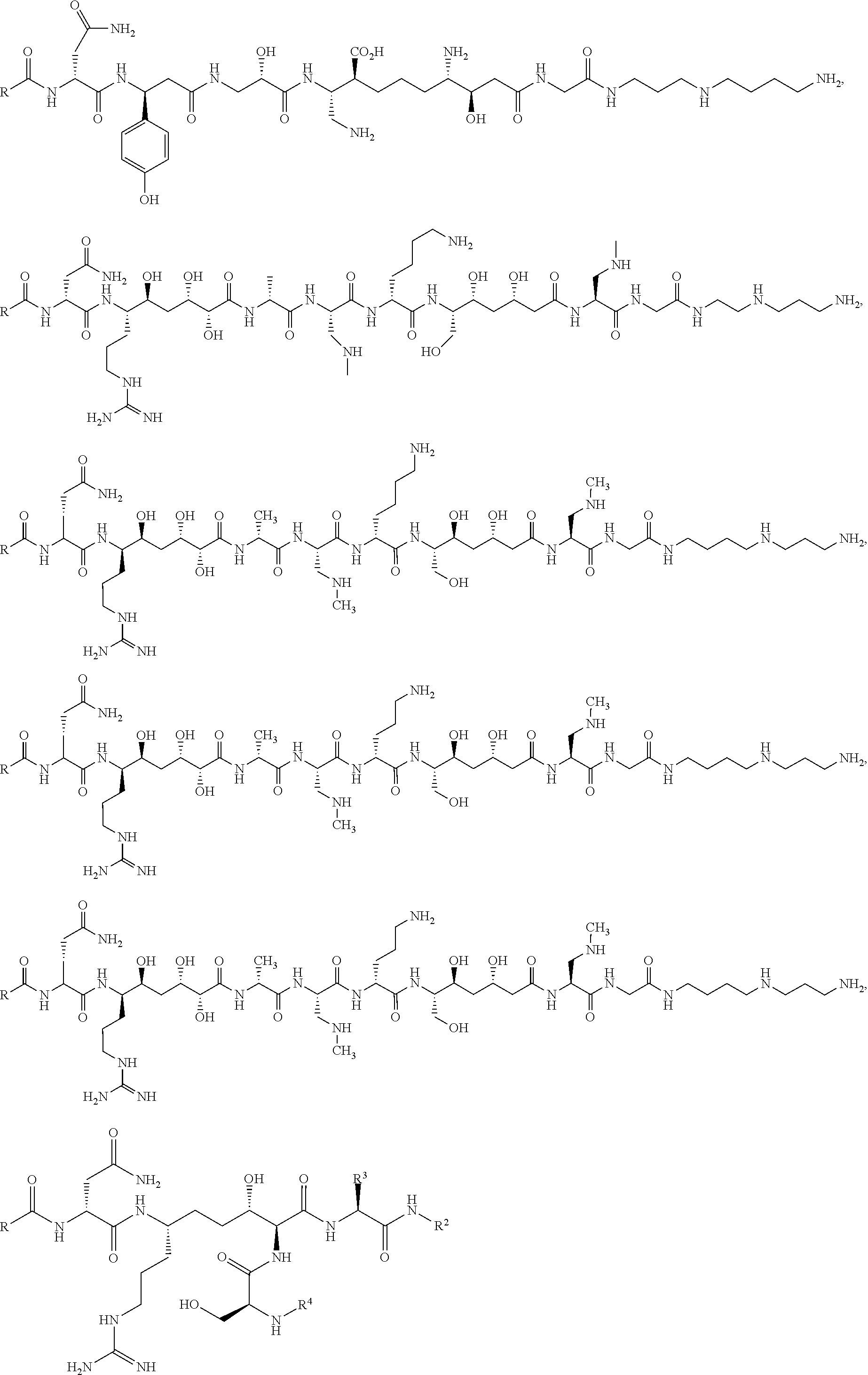 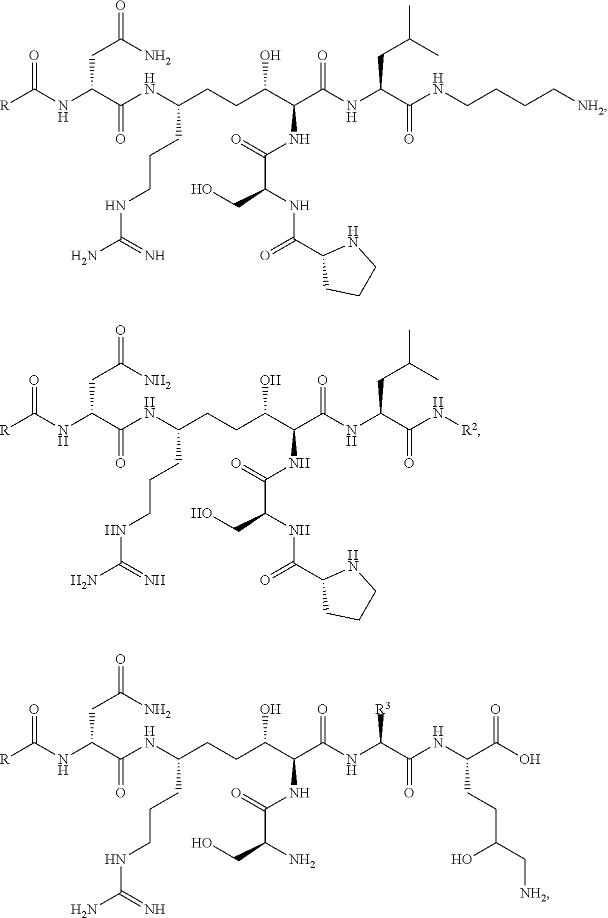
isomers, stereoisomers, enantiomers, diastereomers, salts, hydrates, and solvates of any one or more of the foregoing, and any combination of any of the foregoing, wherein
R is a substituted or unsubstituted aliphatic group, wherein the aliphatic group is a linear substituted or unsubstituted aliphatic group or a branched substituted or unsubstituted aliphatic group;
R² is an amino acid residue, a substituted or unsubstituted aliphatic group, or H;
R³ is a substituted or unsubstituted aliphatic group or an amino acid side chain; and
R⁴ is a substituted or unsubstituted acyl group, an amino acid residue, or H.

2. The method according to claim 1, wherein the aliphatic groups are 1 to 25 carbon atoms in length and optionally further comprise one or more of the following substituents: one or more aromatic rings, one or more pi bonds, one or more hydroxy groups, and/or one or more halogens.

3. The method according to claim 1, wherein the aliphatic groups are substituted with one or more fluoro groups.

4. The method according to claim 1, wherein R has the following structure 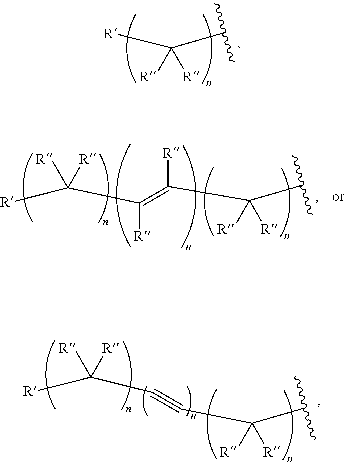
wherein R′ is hydrogen, a halogen, an aryl group, a methyl group, a halogenated methyl group, an amino group, or a hydroxy, and each R″ is independently hydrogen, a halogen, an aryl group, a methyl group, a halogenated methyl group, or a hydroxy.

5. The method according to claim 1, wherein R has the following structure: 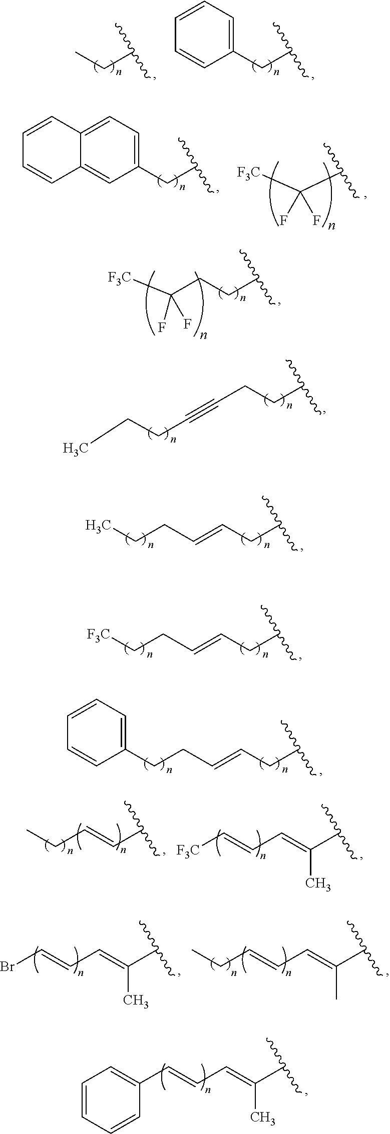 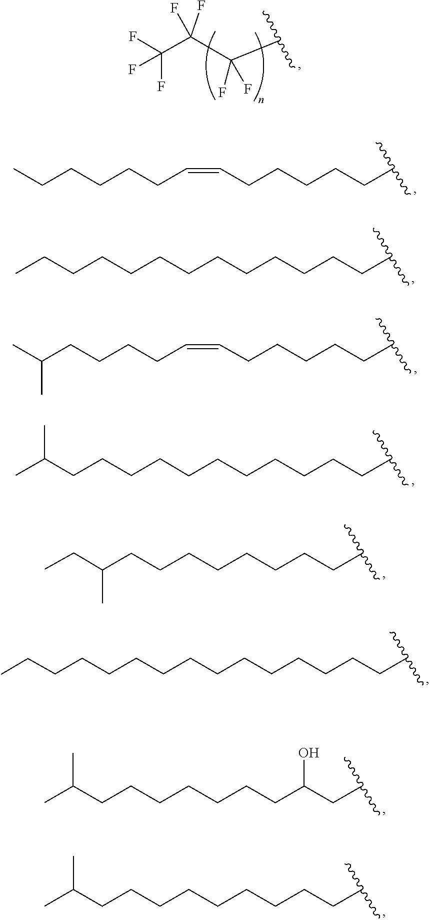 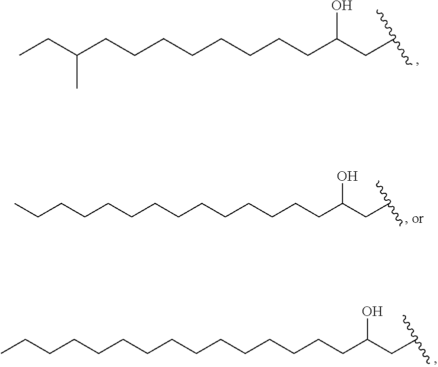
wherein each n is independently 0 to 18.

6. The method according to claim 1, wherein R² is

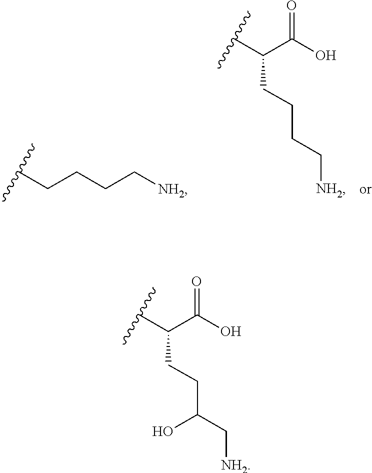 [SMILES: *CCCCN ; *[C@@H](CCCCN)C(=O)O ; *[C@@H](CCC(O)CN)C(=O)O]

7. The method according to claim 1, wherein R³ is

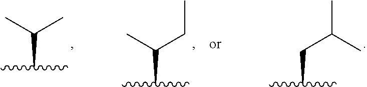

8. The method according to claim 1, wherein R⁴ is H or

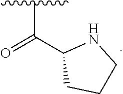 [SMILES: *C(=O)[C@H]1CCCN1]

9. The method according claim 1, wherein the prodrug antibiotic has the following structure:

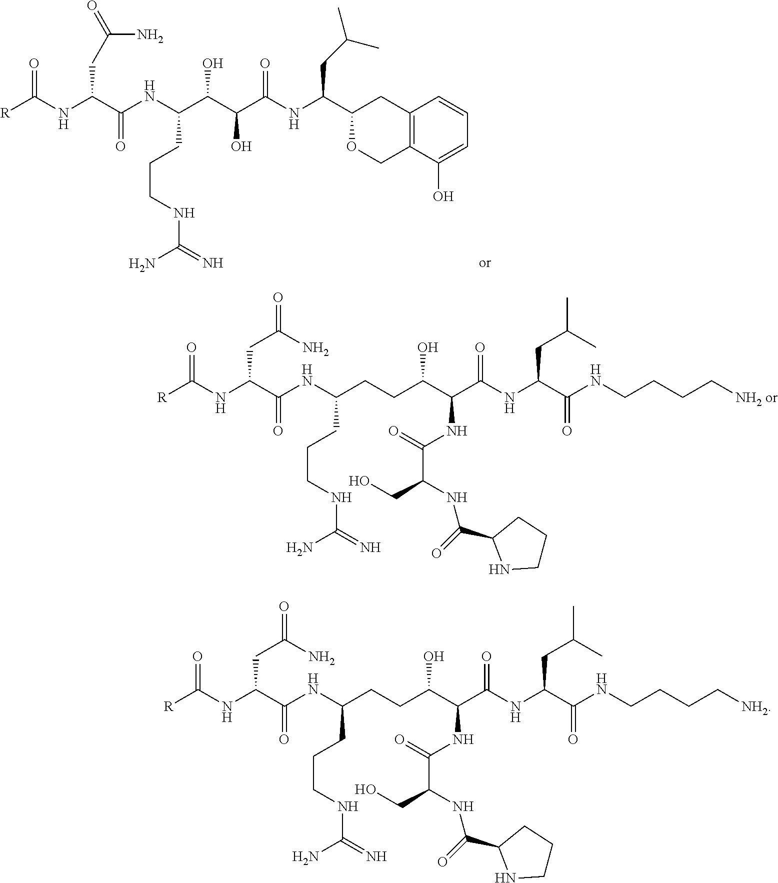

10. The method according to claim 9, wherein the prodrug antibiotic is: 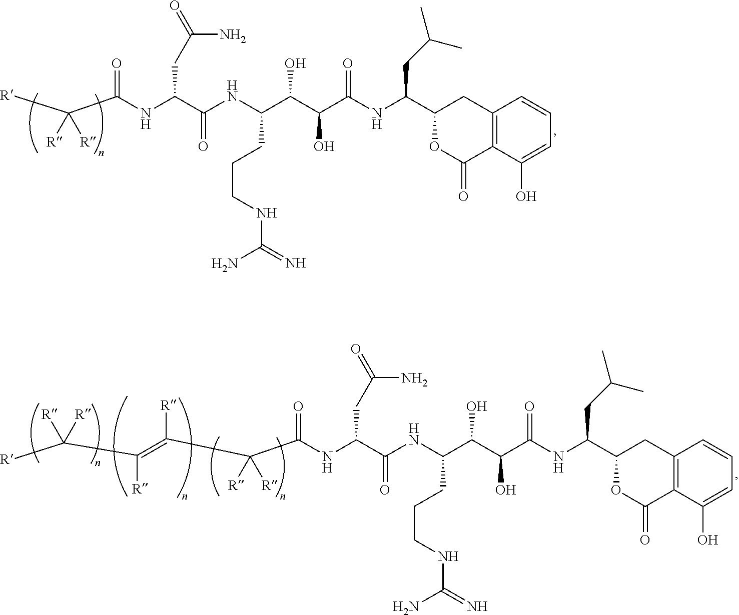 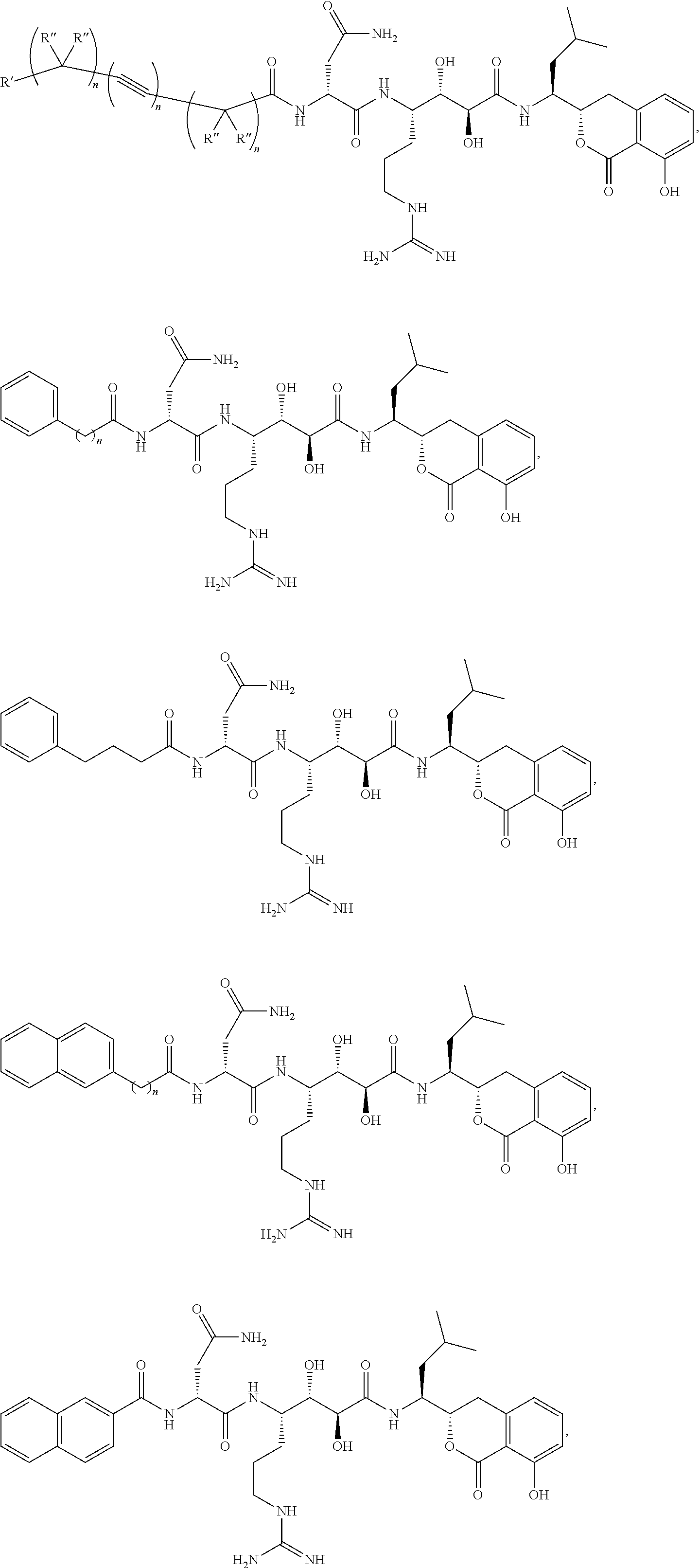 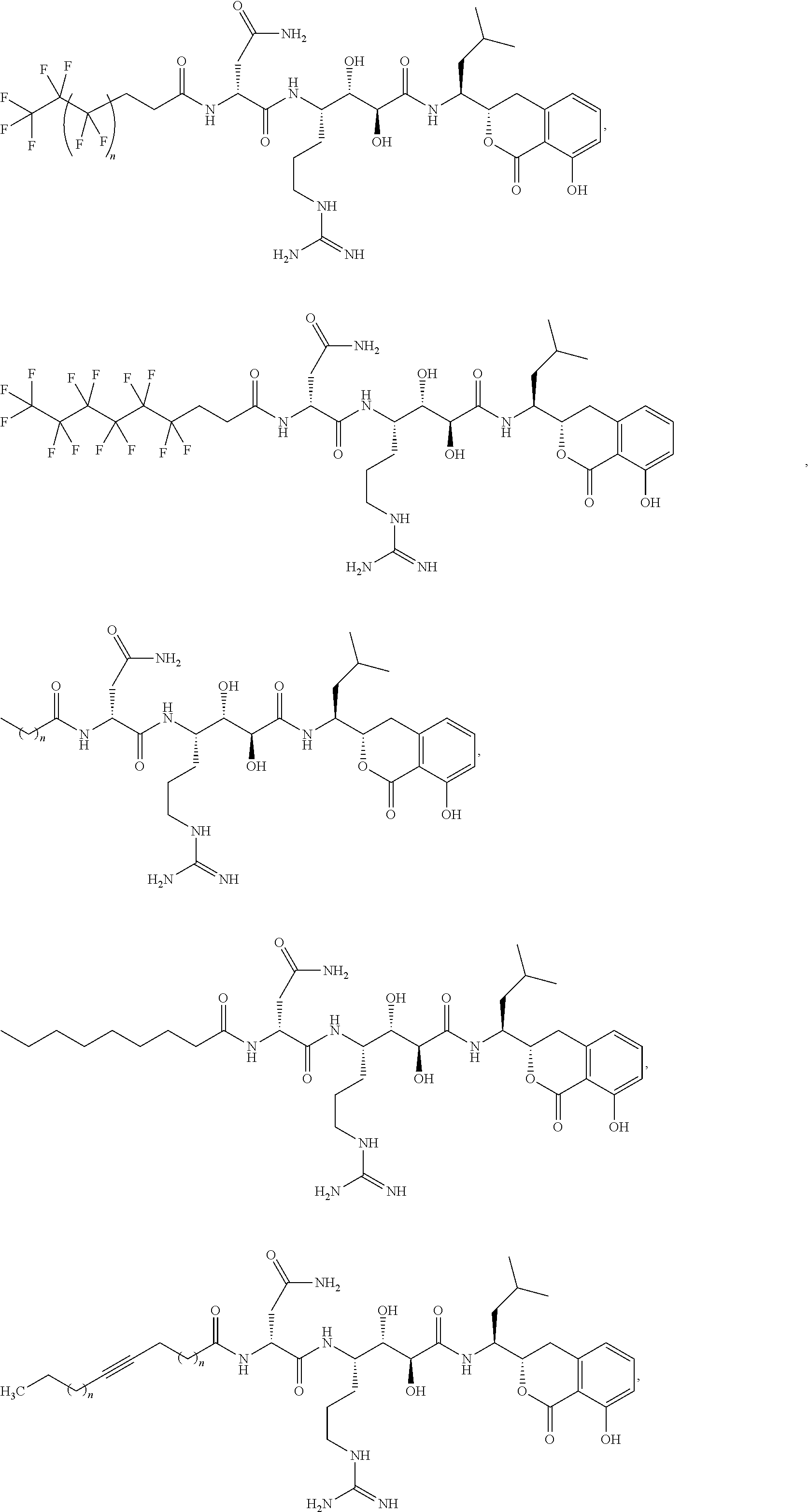 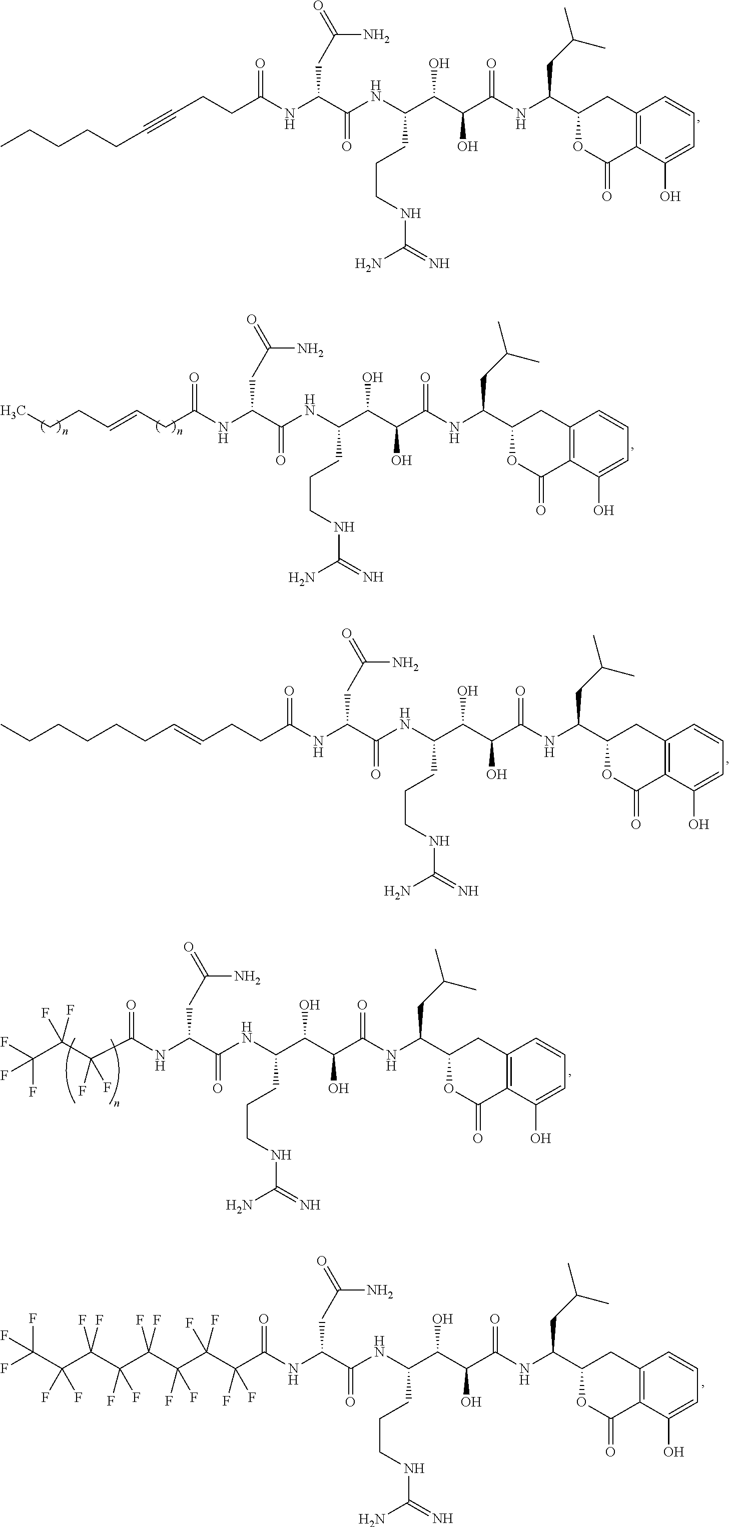 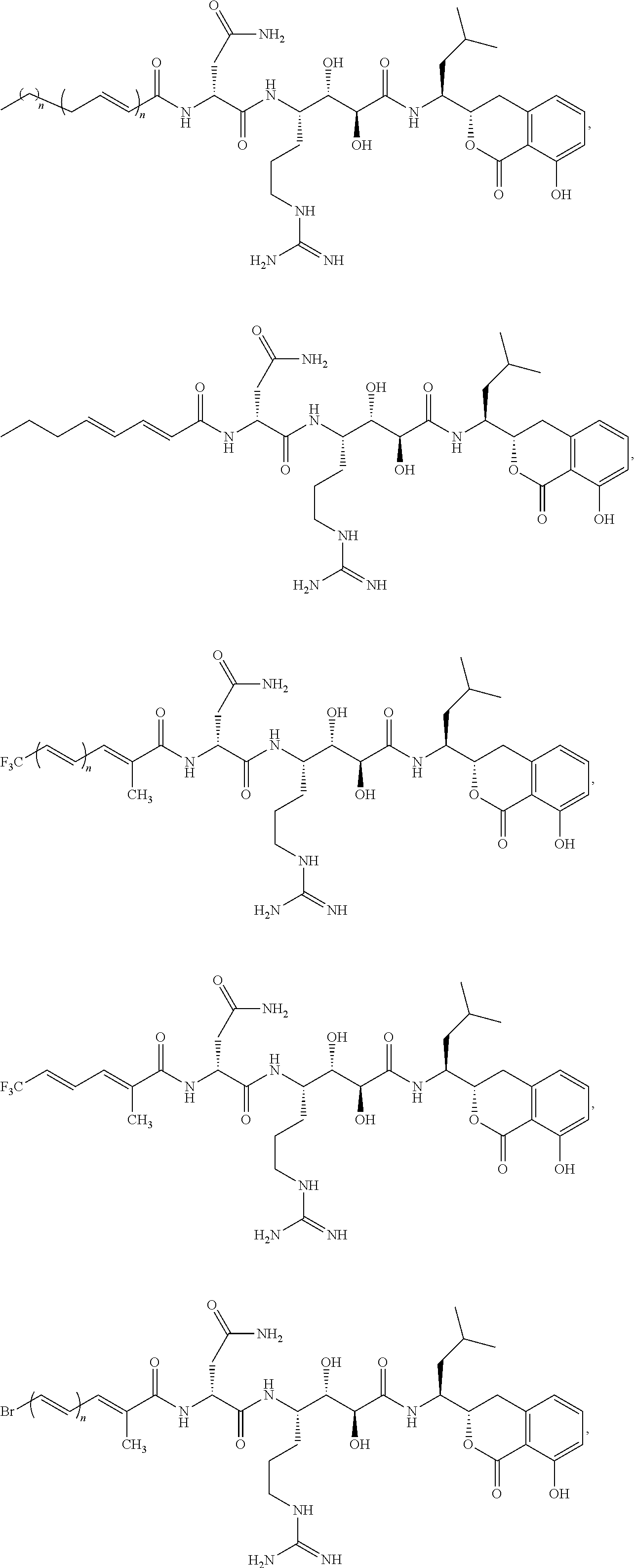 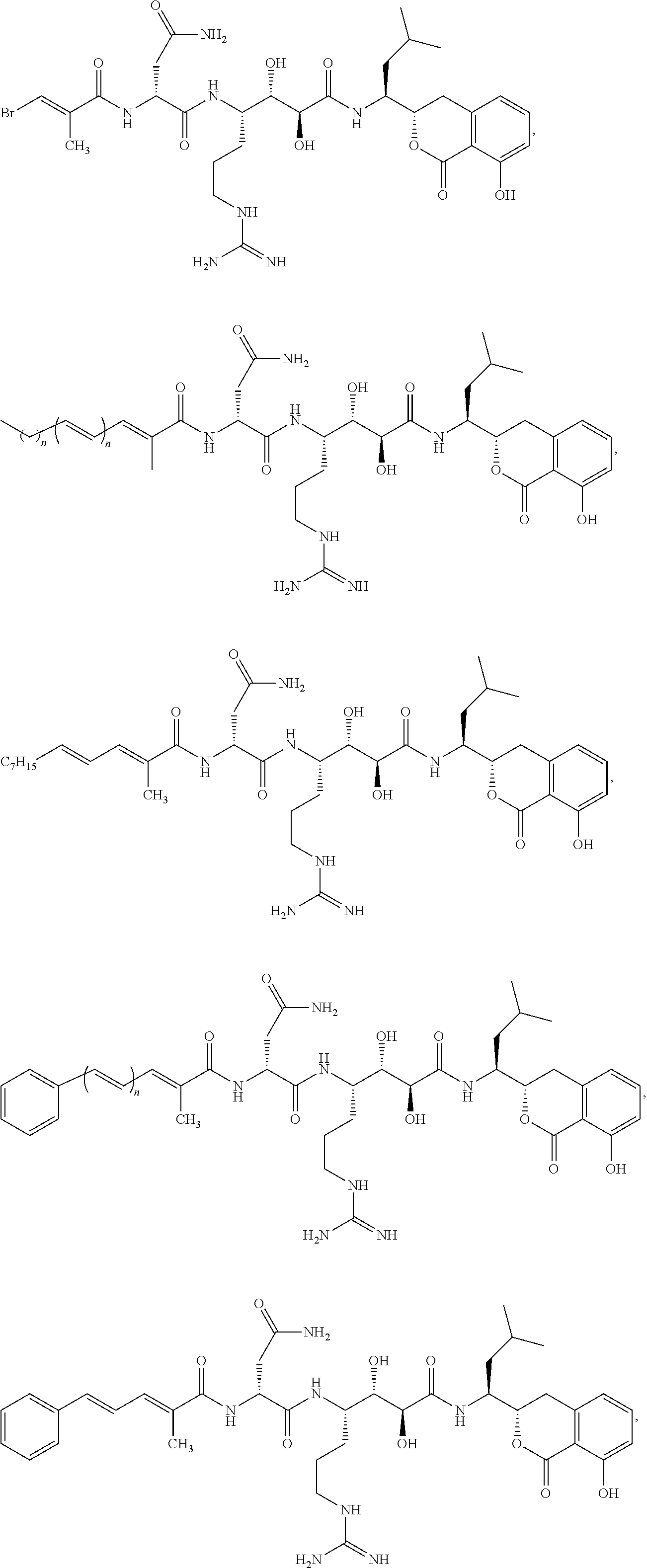 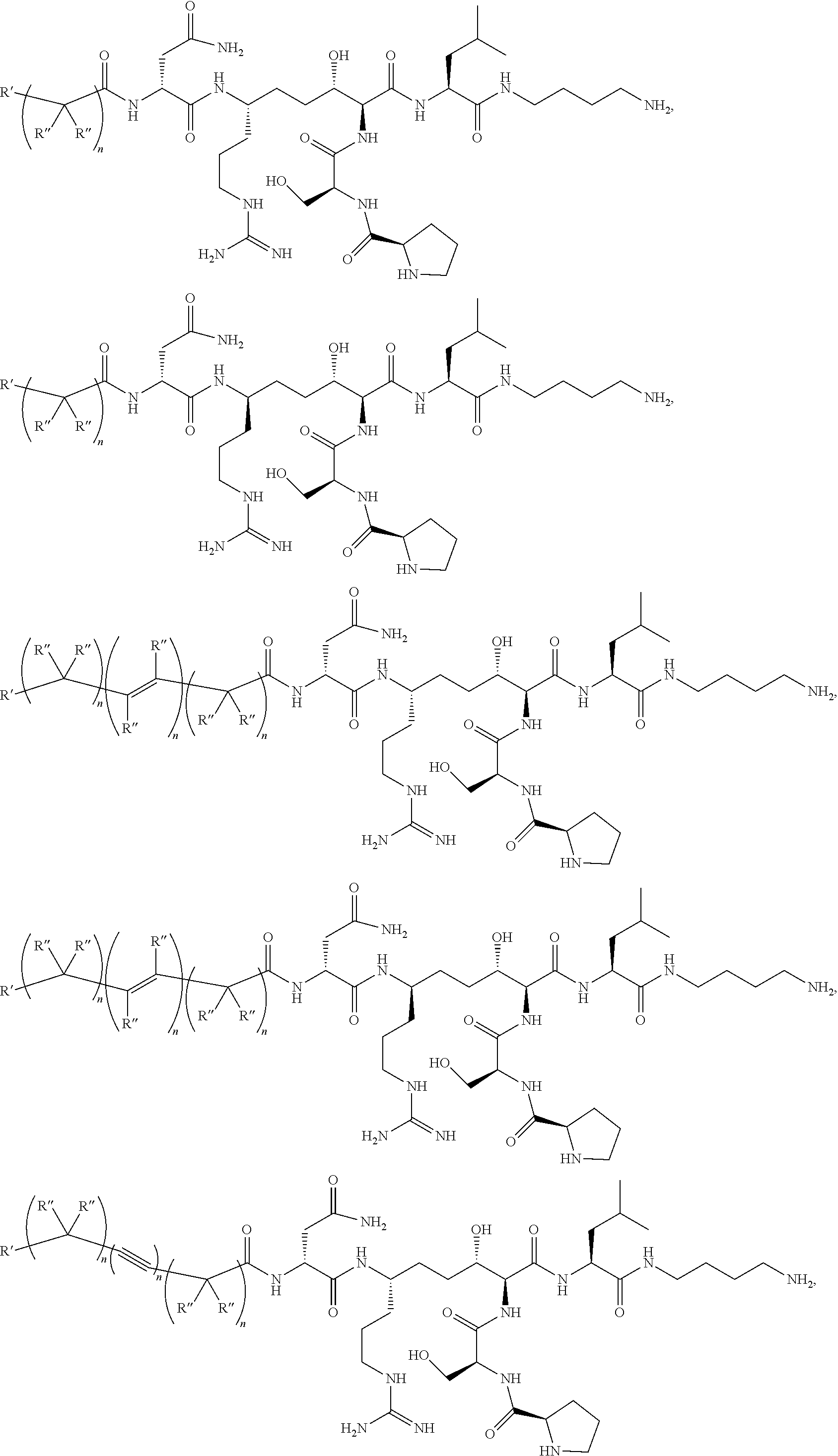 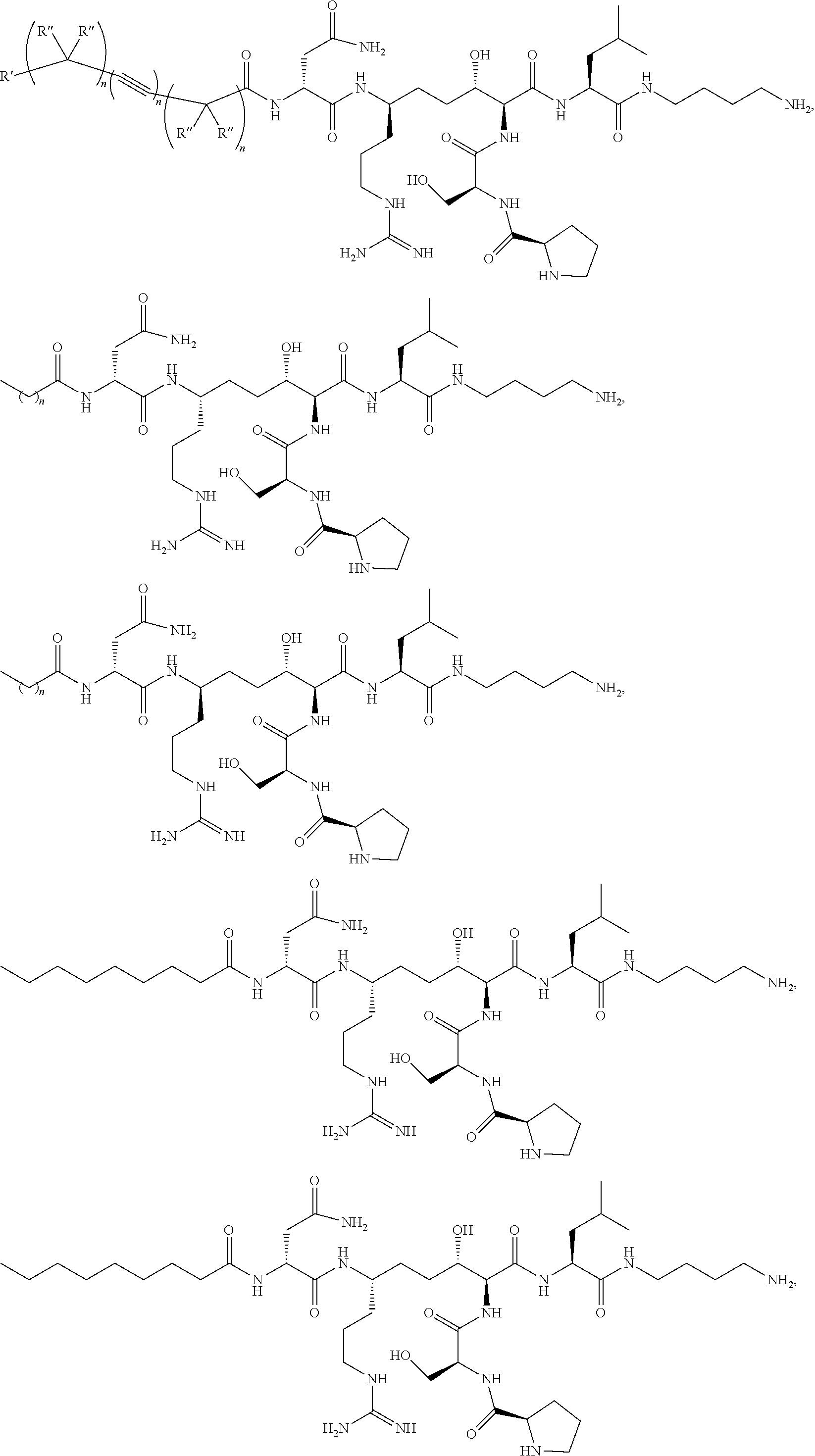 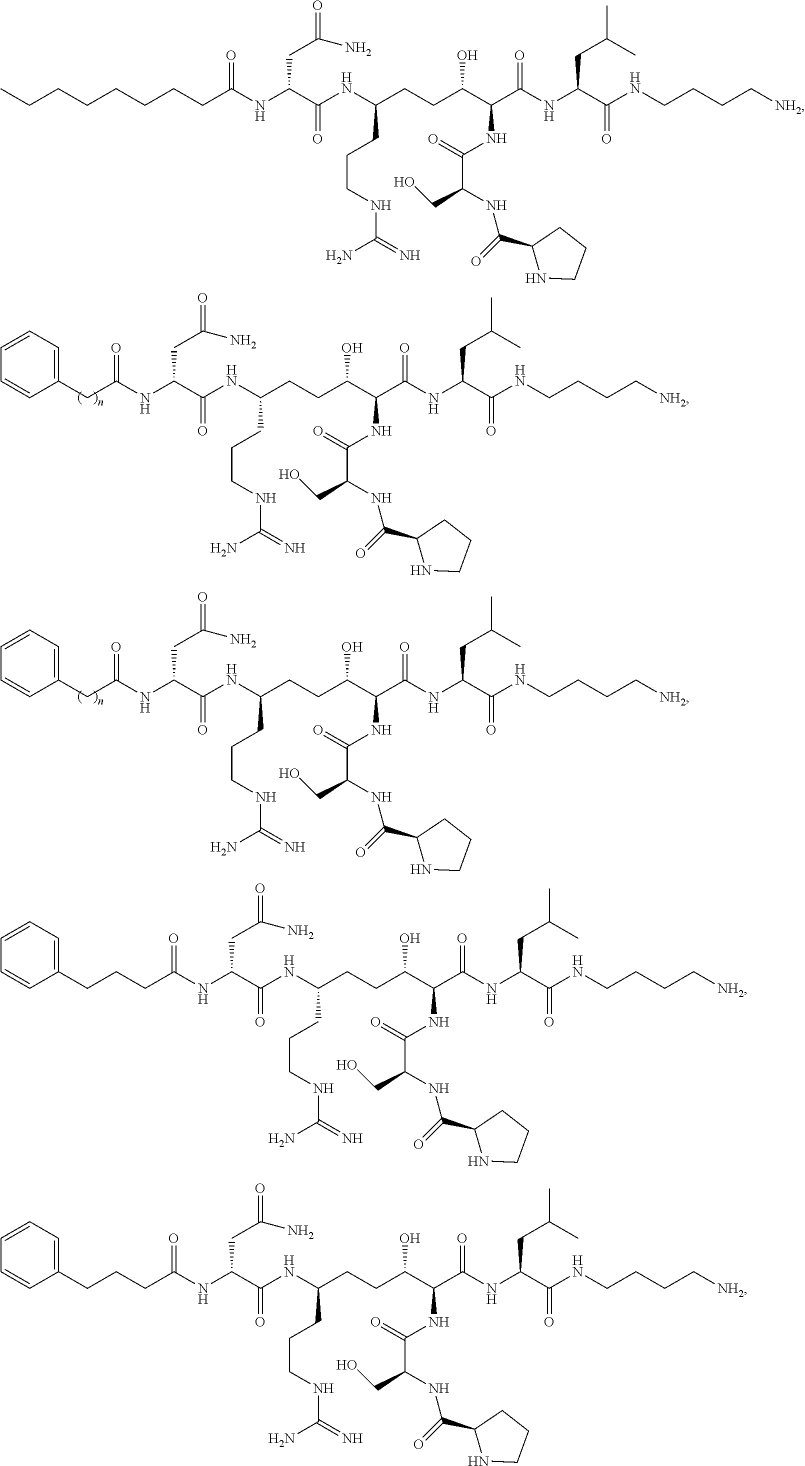 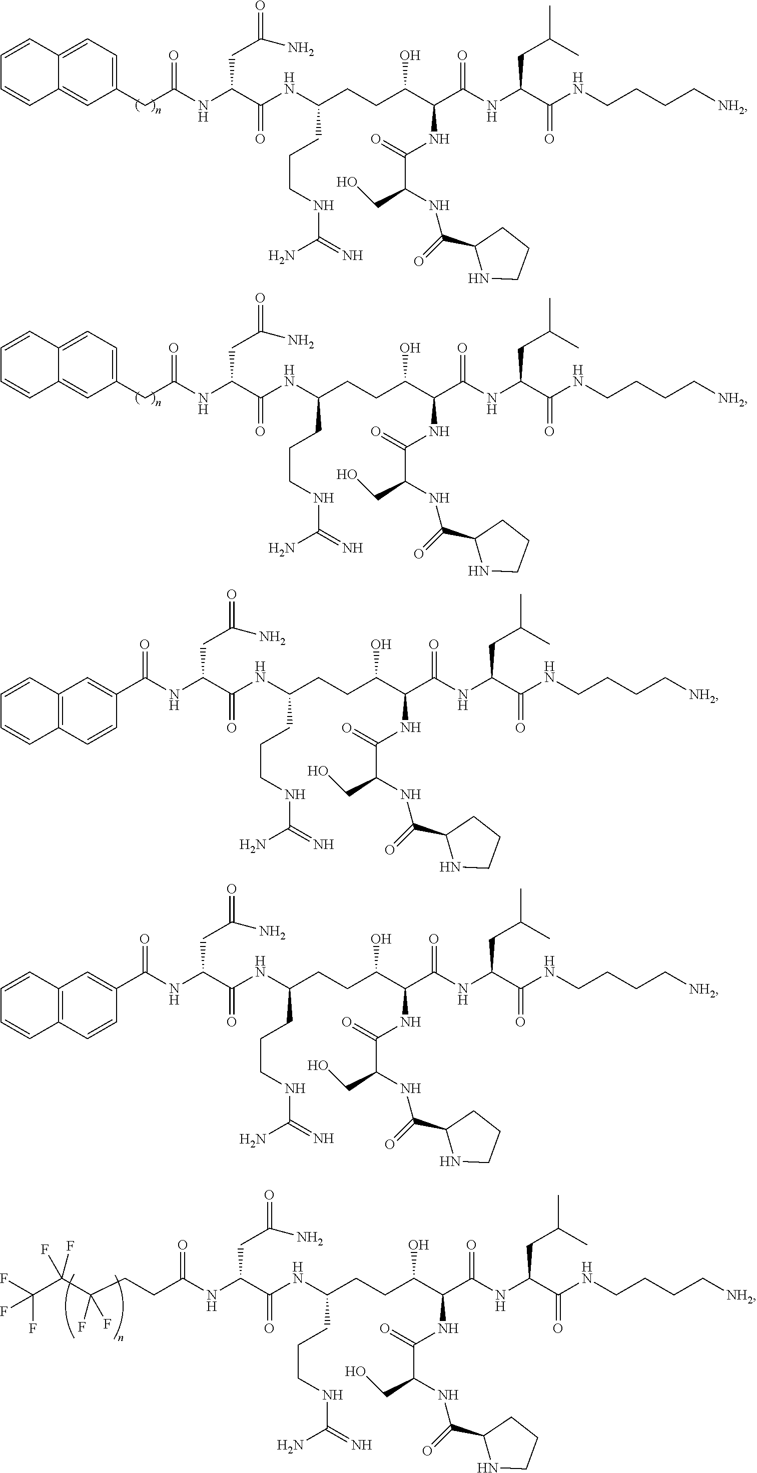 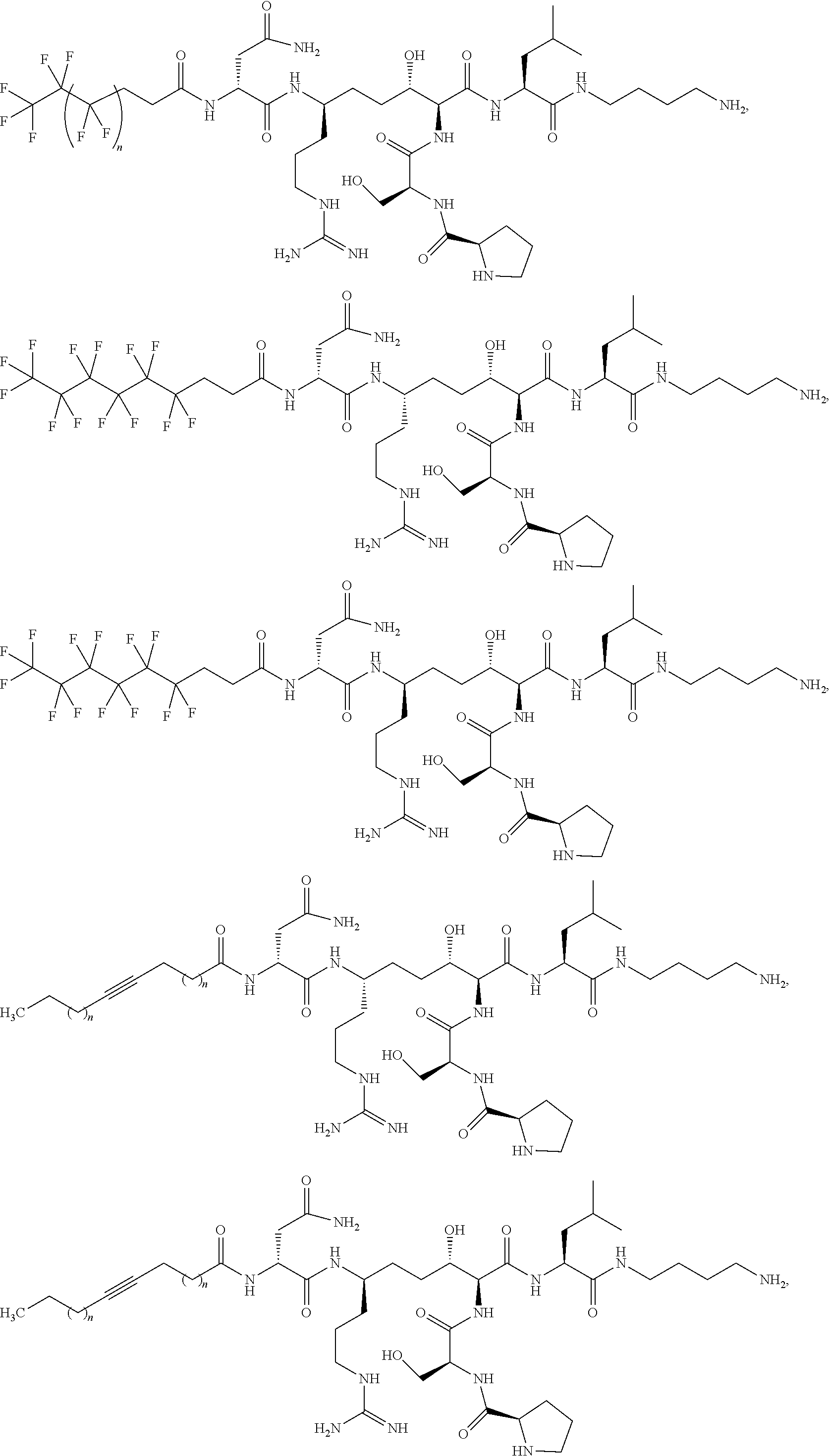 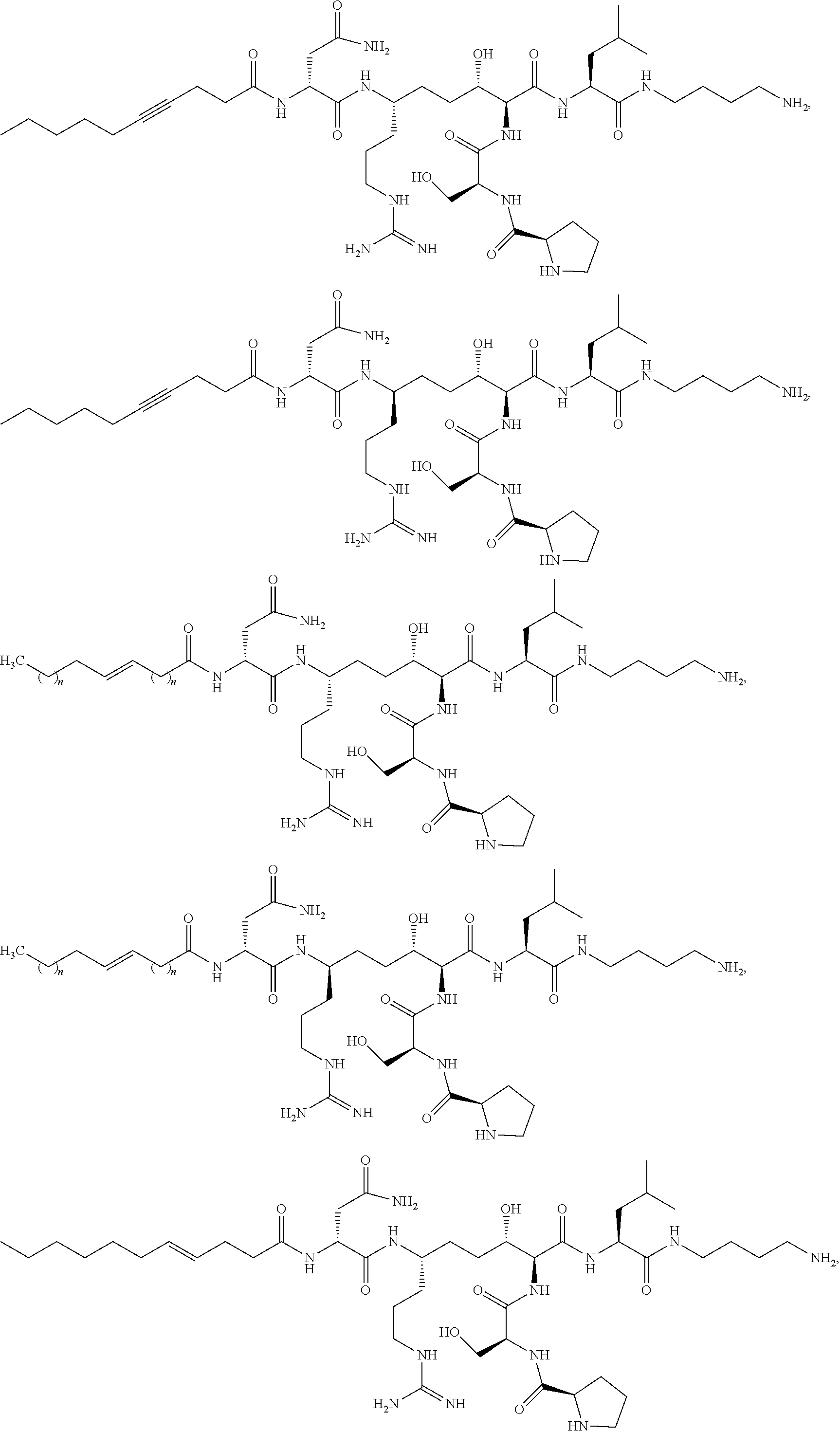 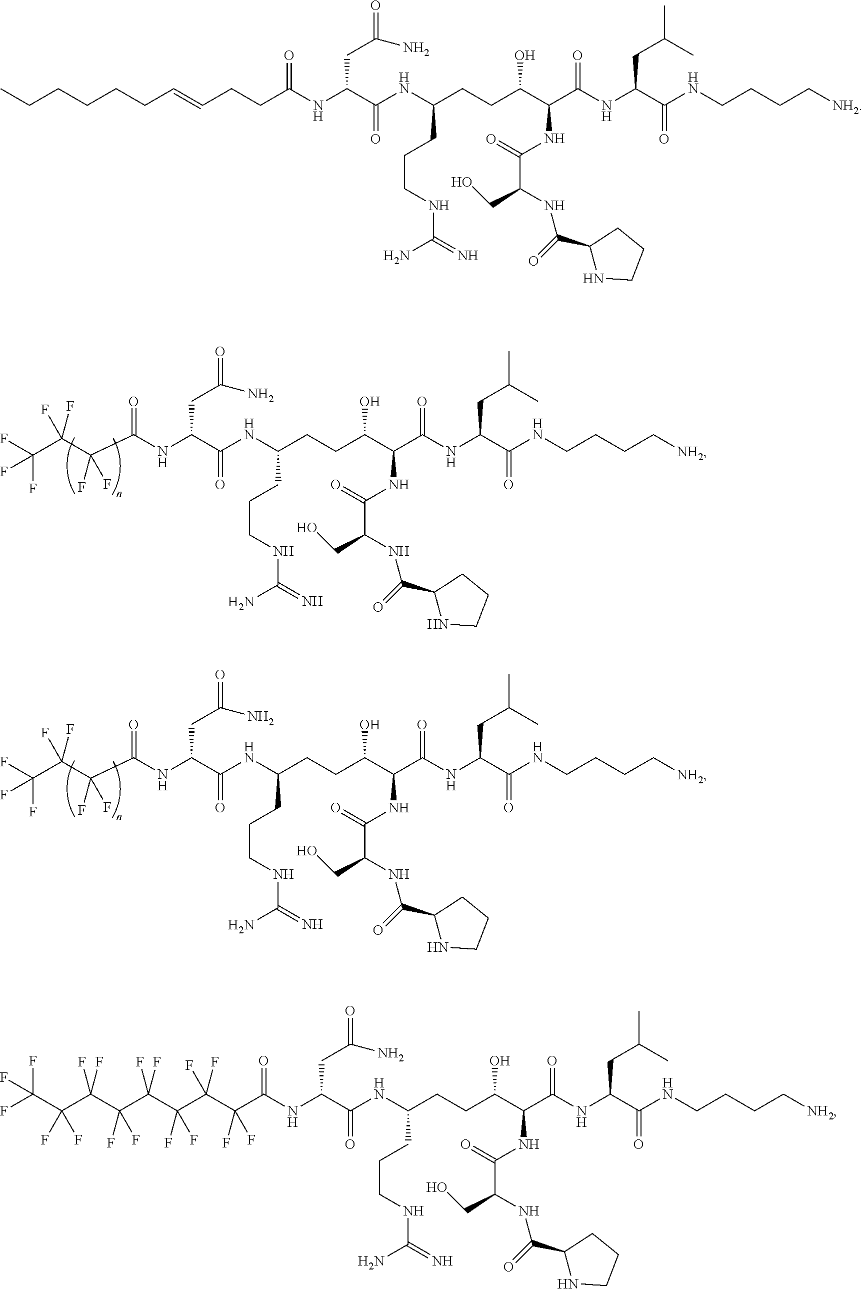 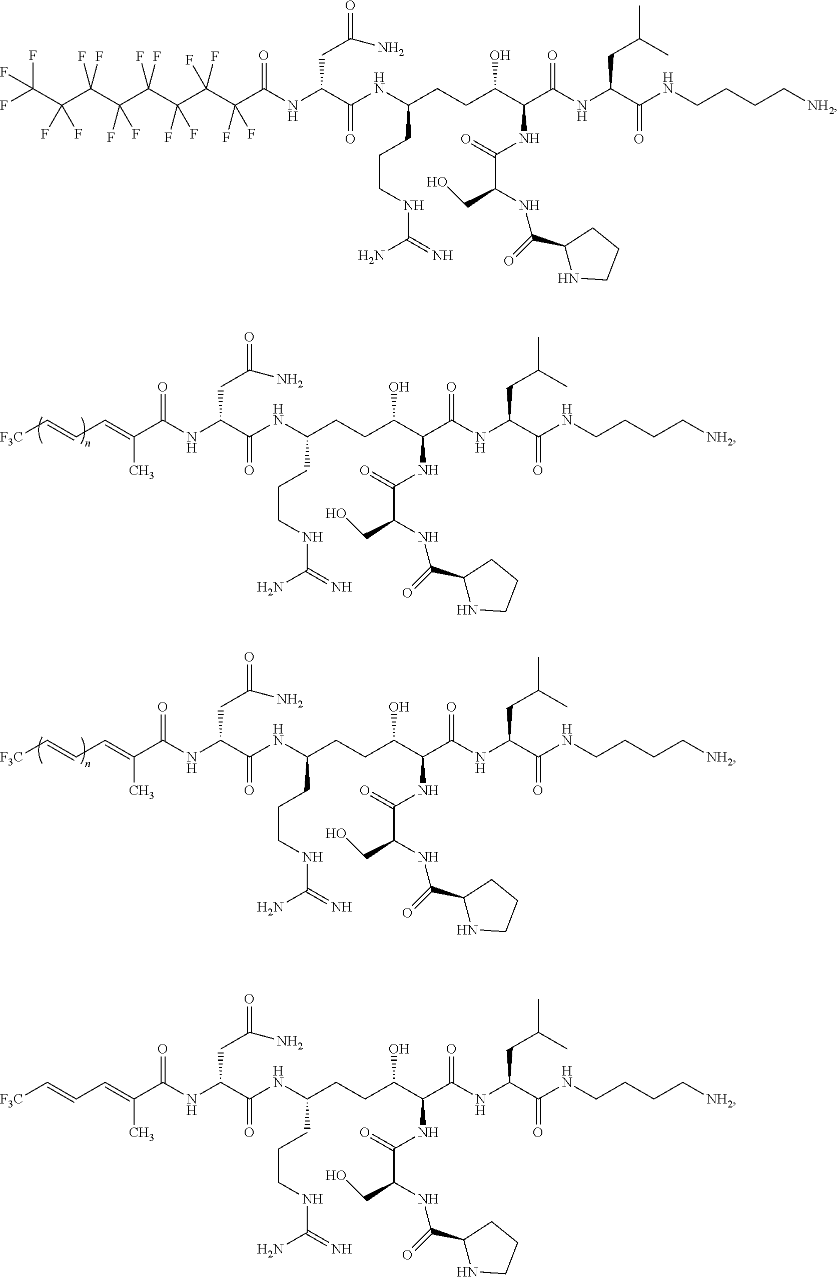 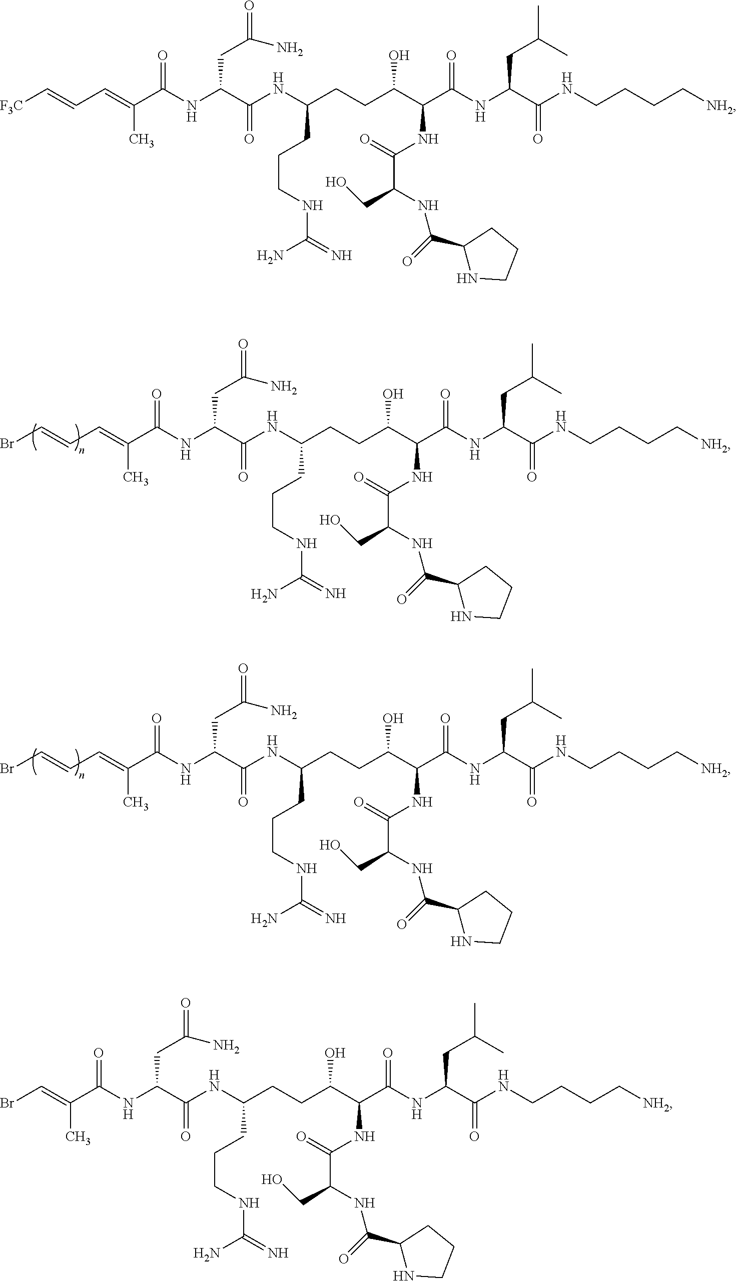 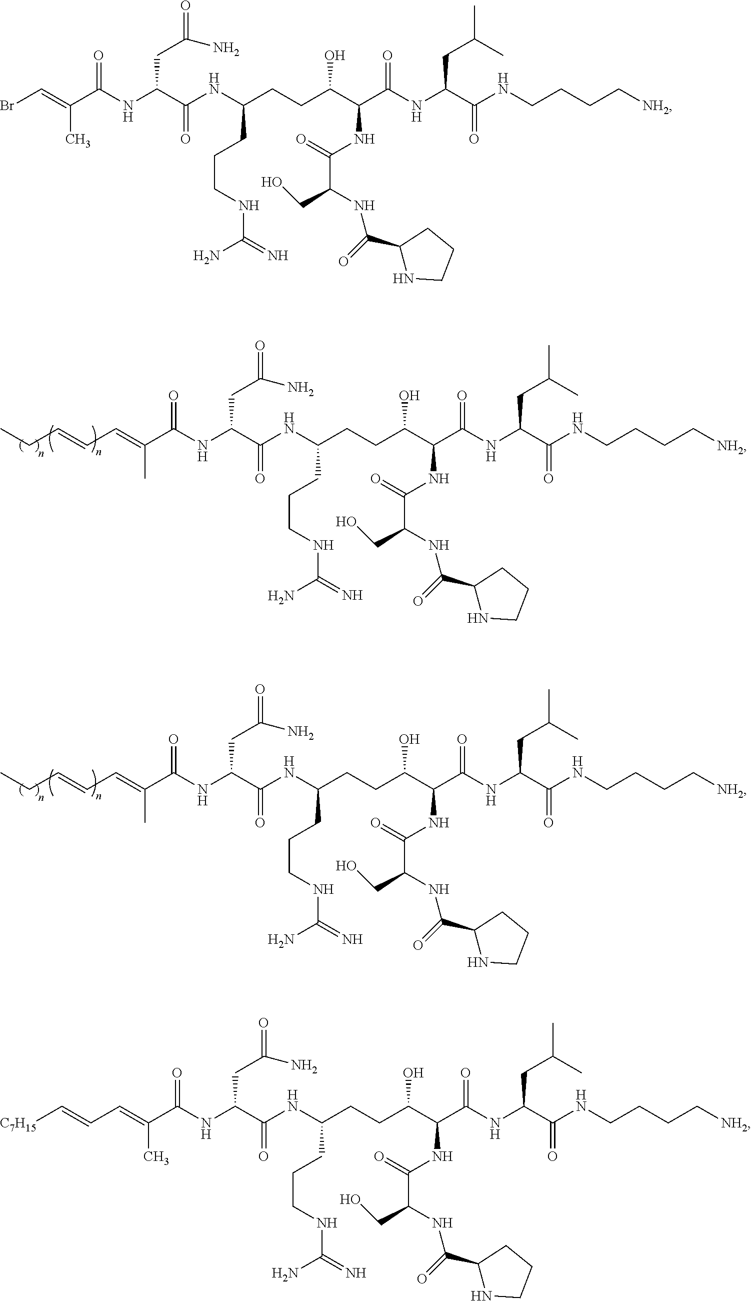 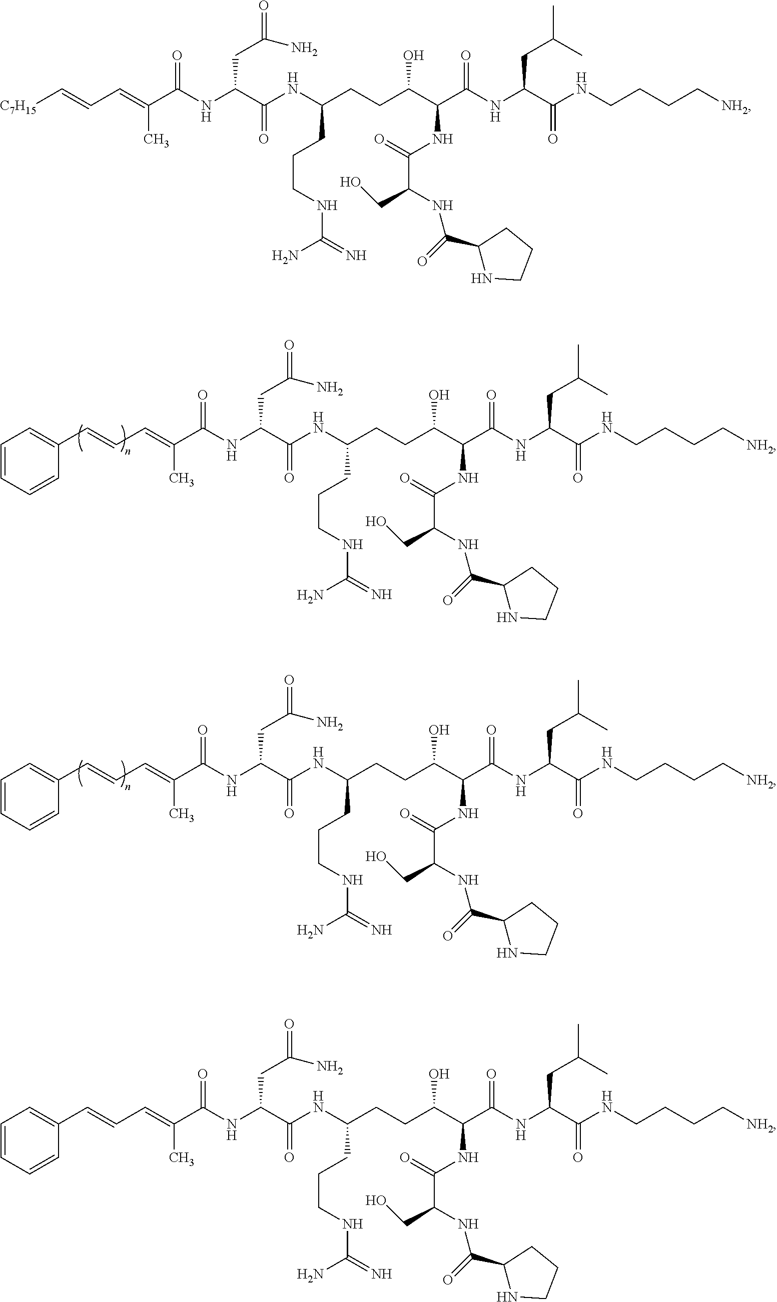 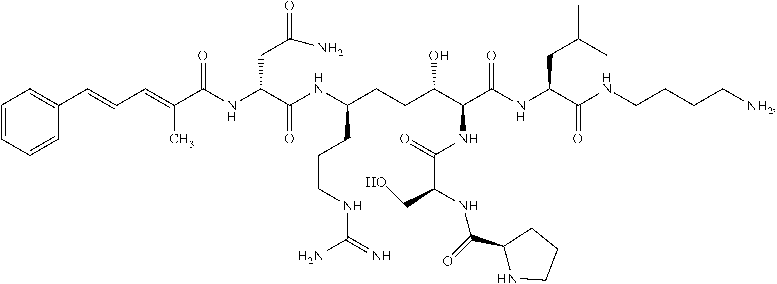 [SMILES: C/C(=C\C=C\c1ccccc1)C(=O)N[C@H](CC(N)=O)C(=O)N[C@H](CCCNC(=N)N)CC[C@H](O)[C@H](NC(=O)[C@H](CO)NC(=O)[C@H]1CCCN1)C(=O)N[C@@H](CC(C)C)C(=O)NCCCCN]
or any combination thereof, wherein n is 0 to 18.

11. The method according claim 1, wherein the prodrug antibiotic has the following structure:

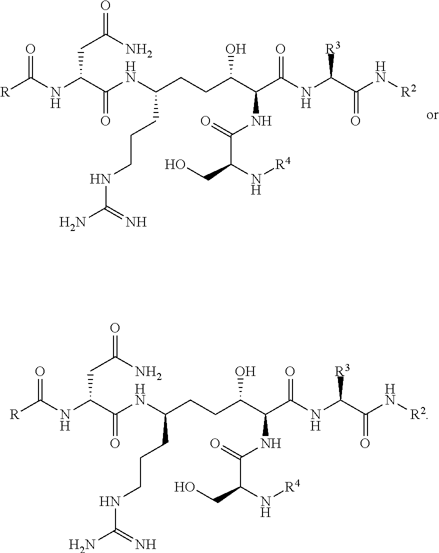

12. The method according to claim 11, wherein the prodrug antibiotic has the following structure:

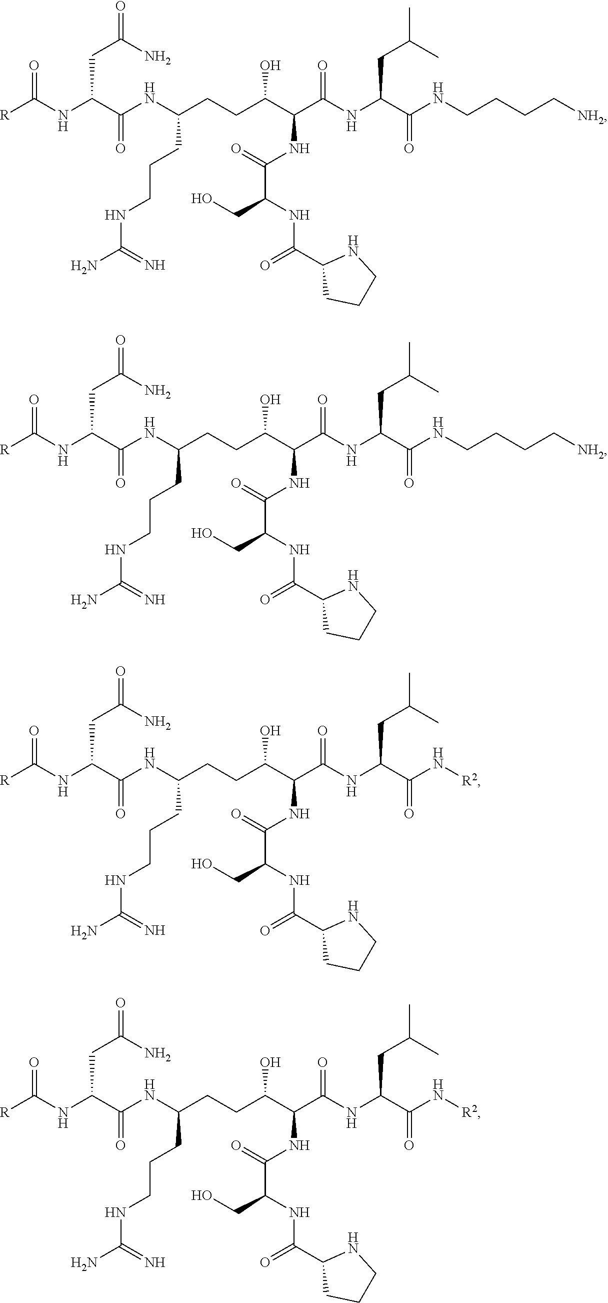

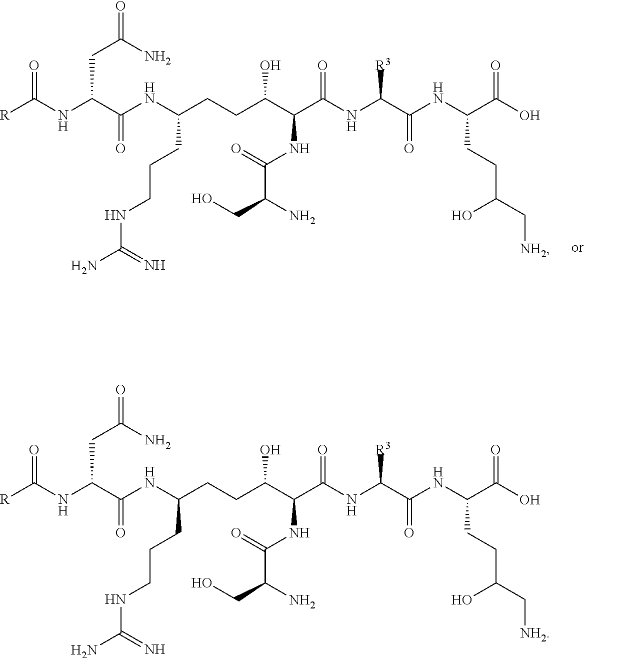

13. The method according to claim 12, wherein the prodrug antibiotic has the following structure:

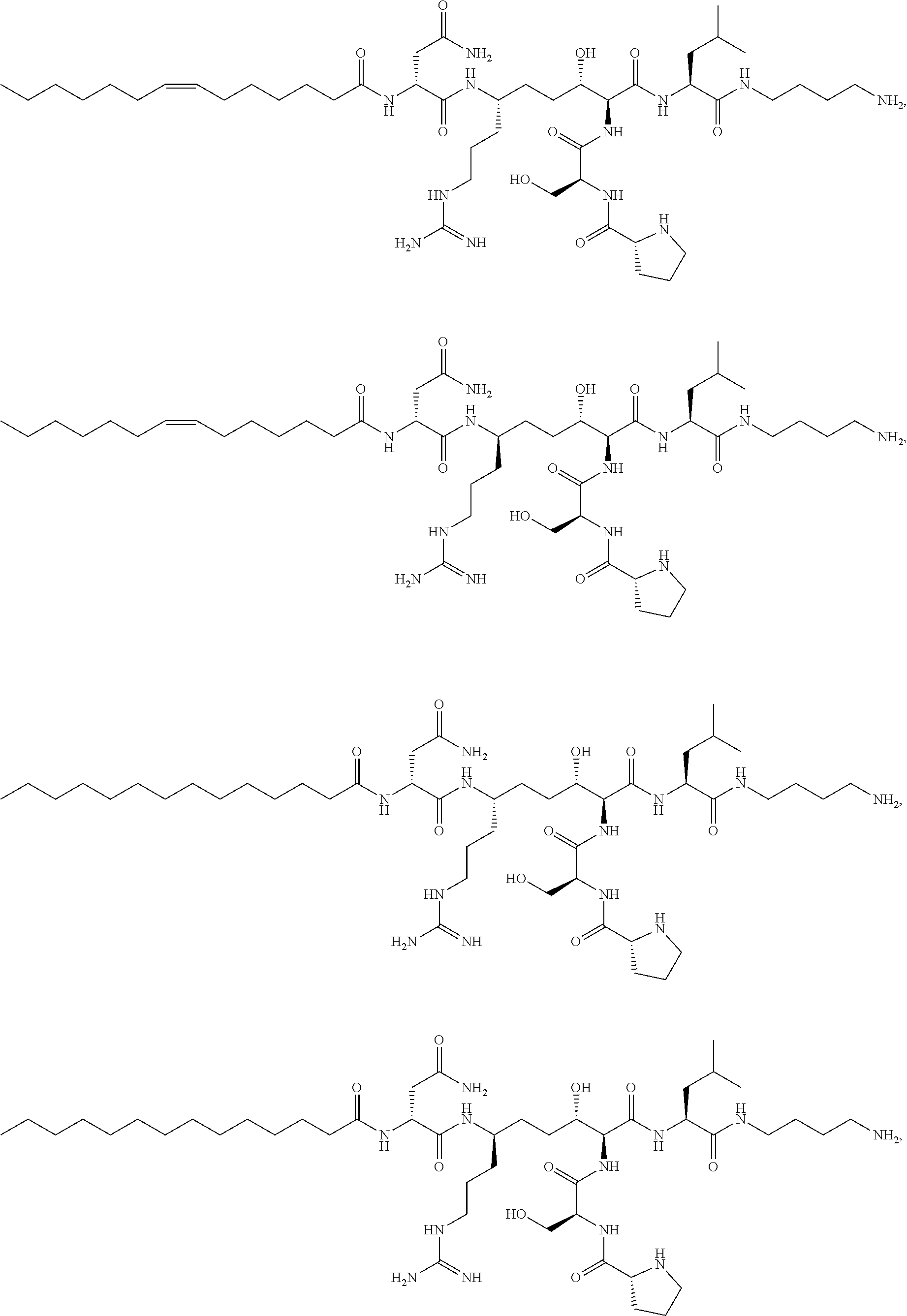

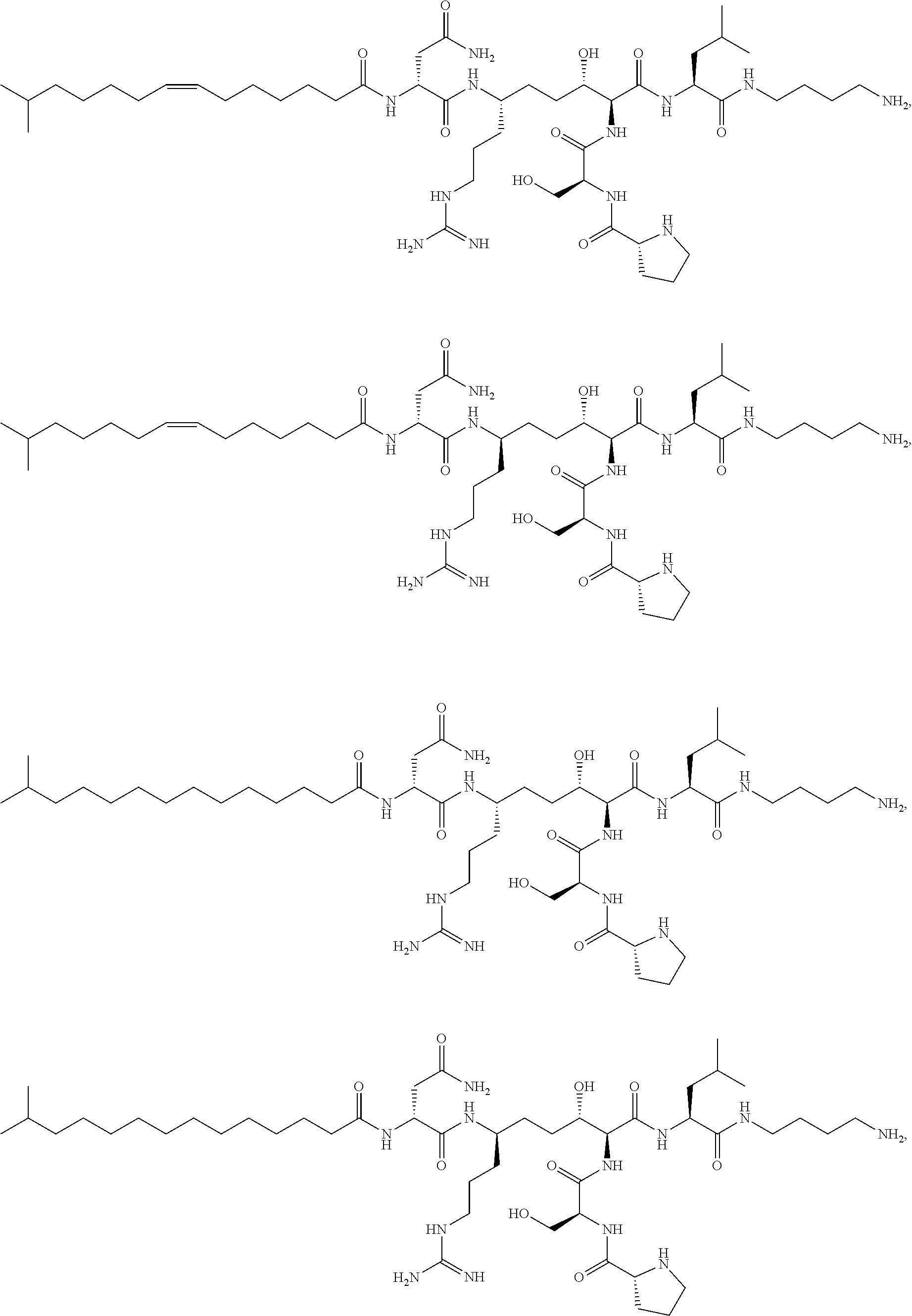

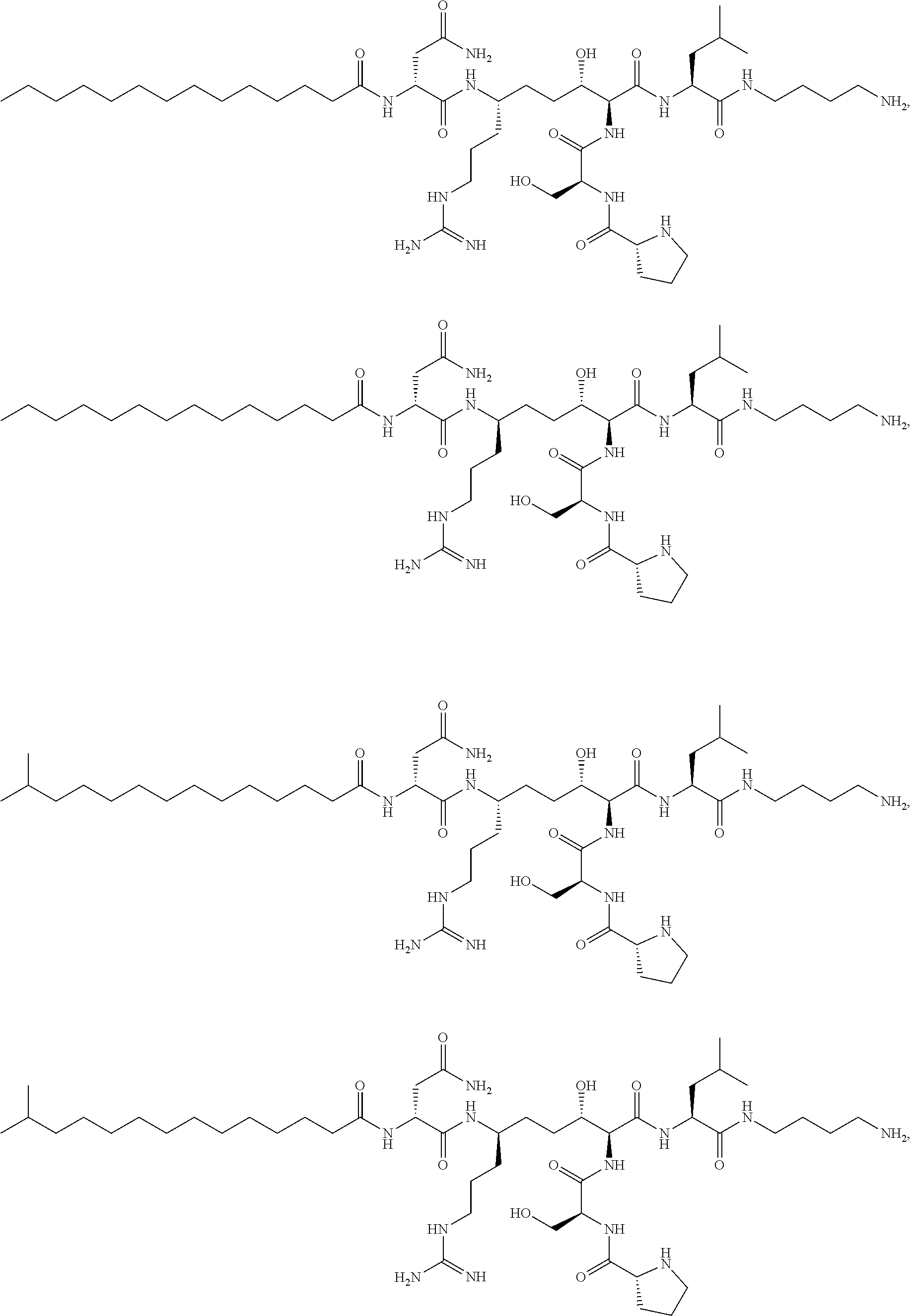

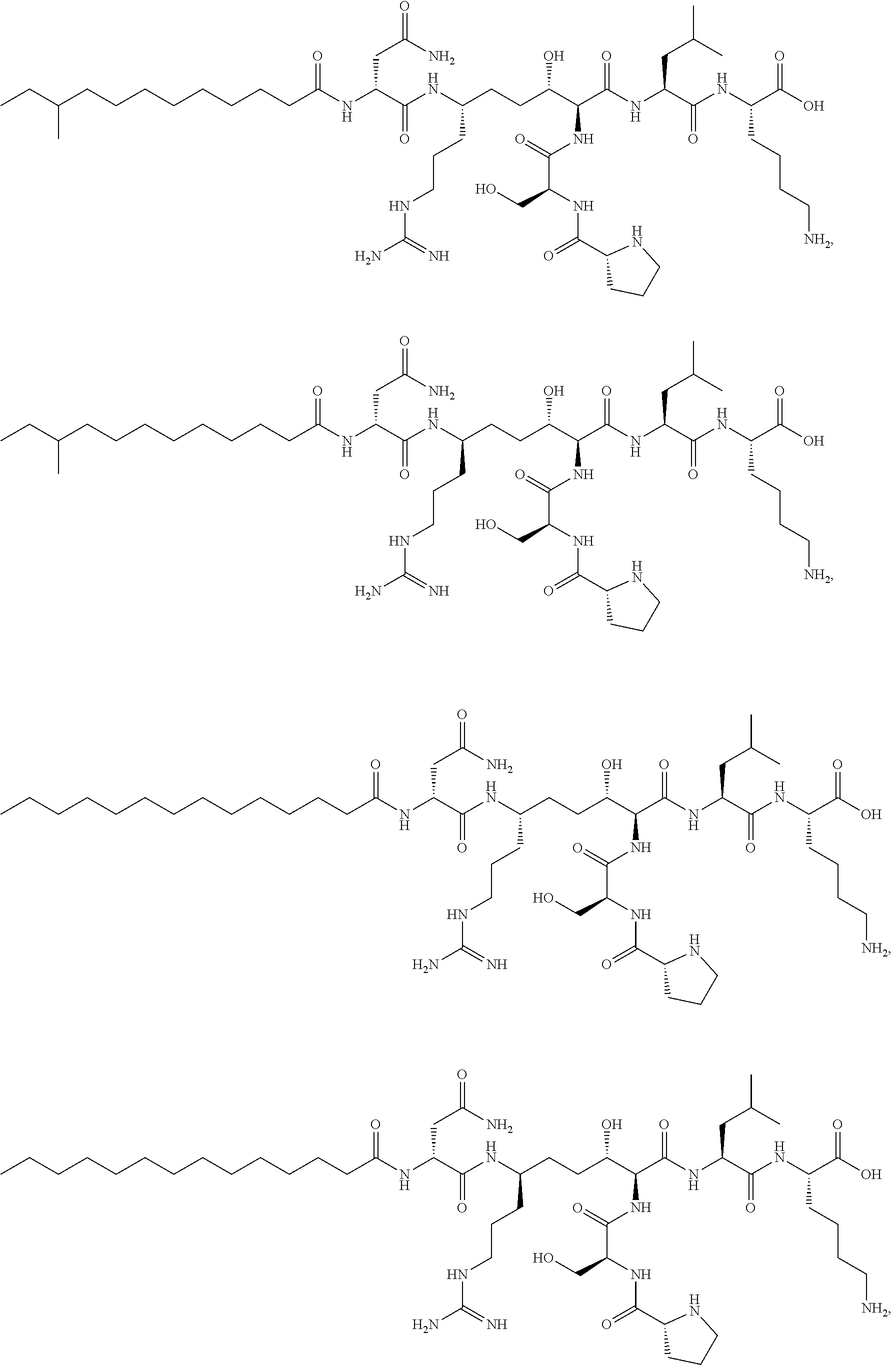

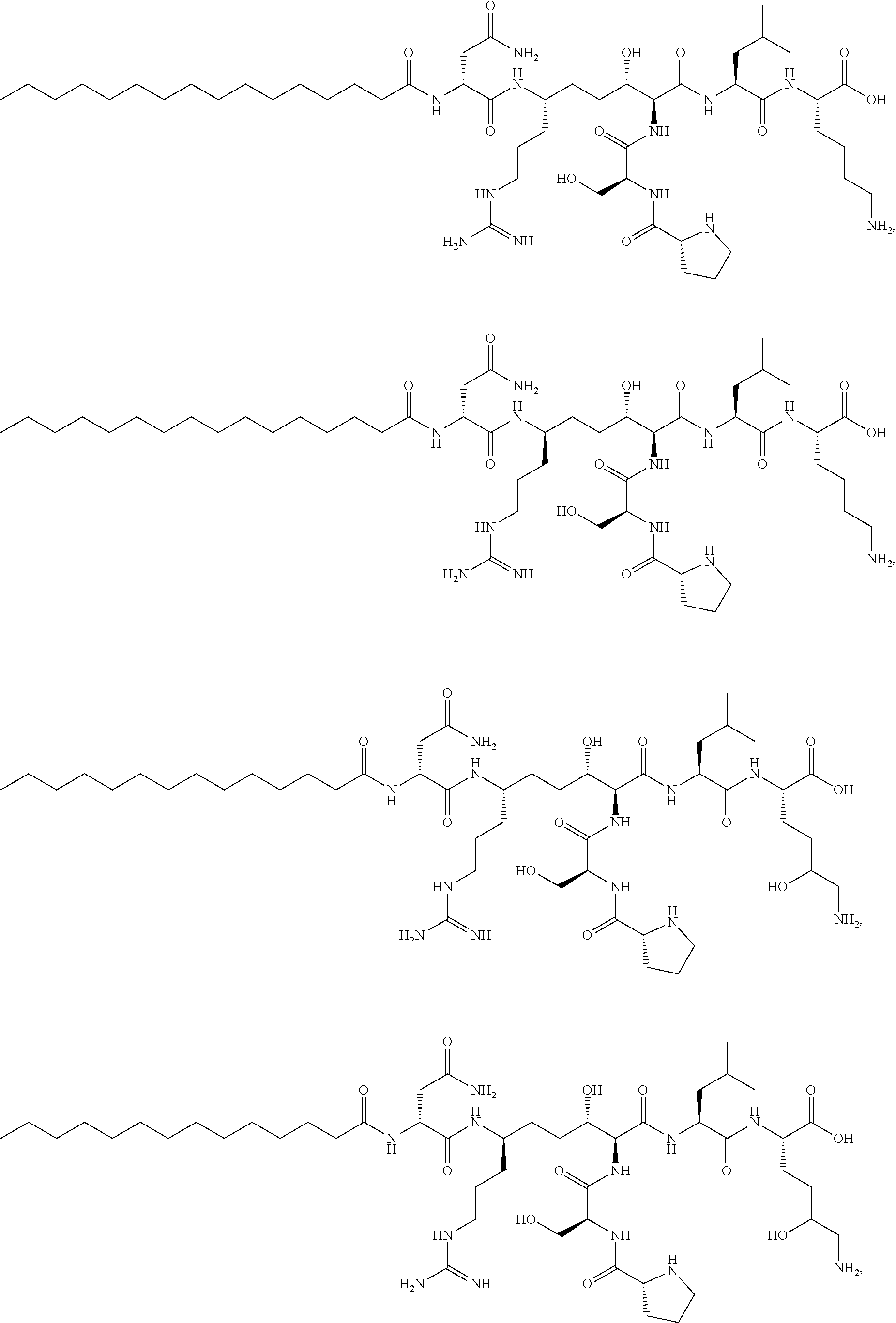

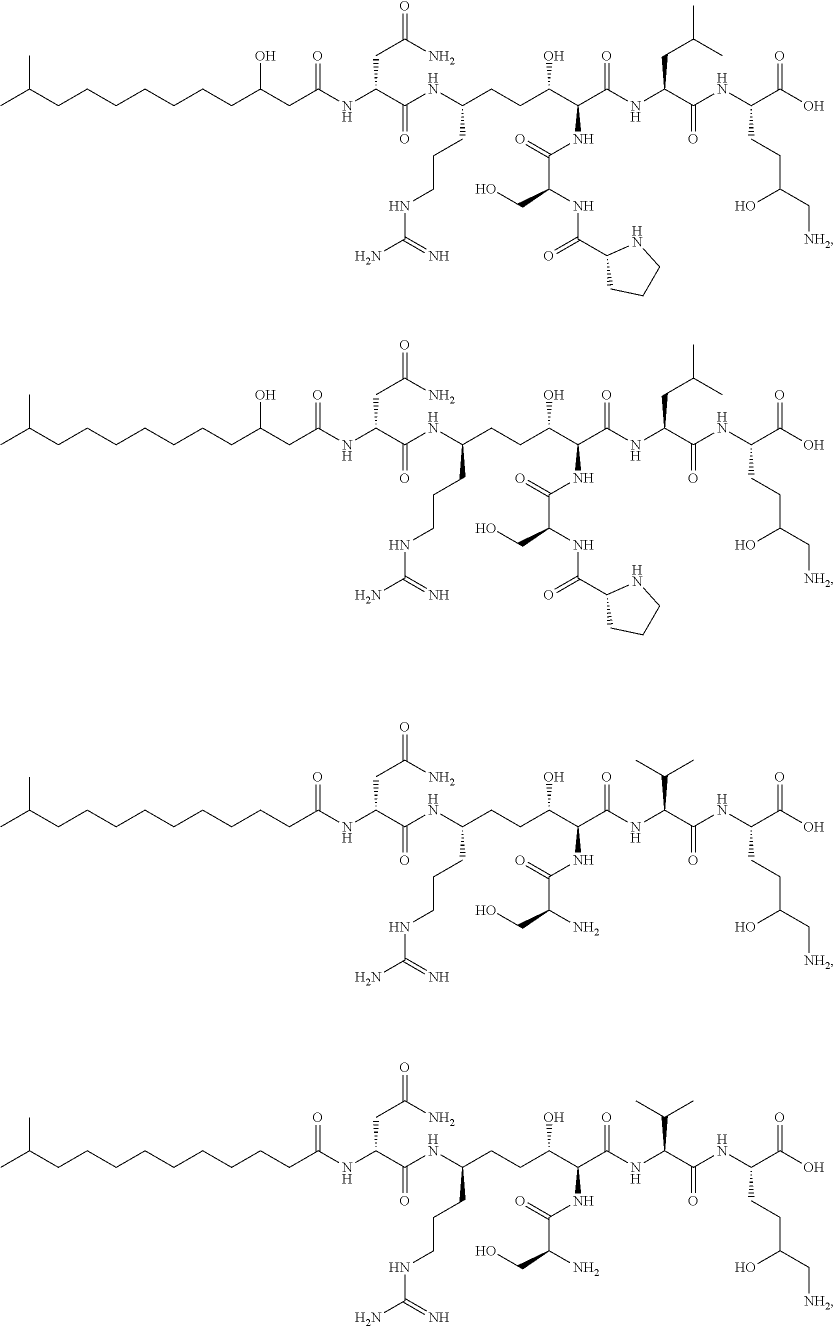

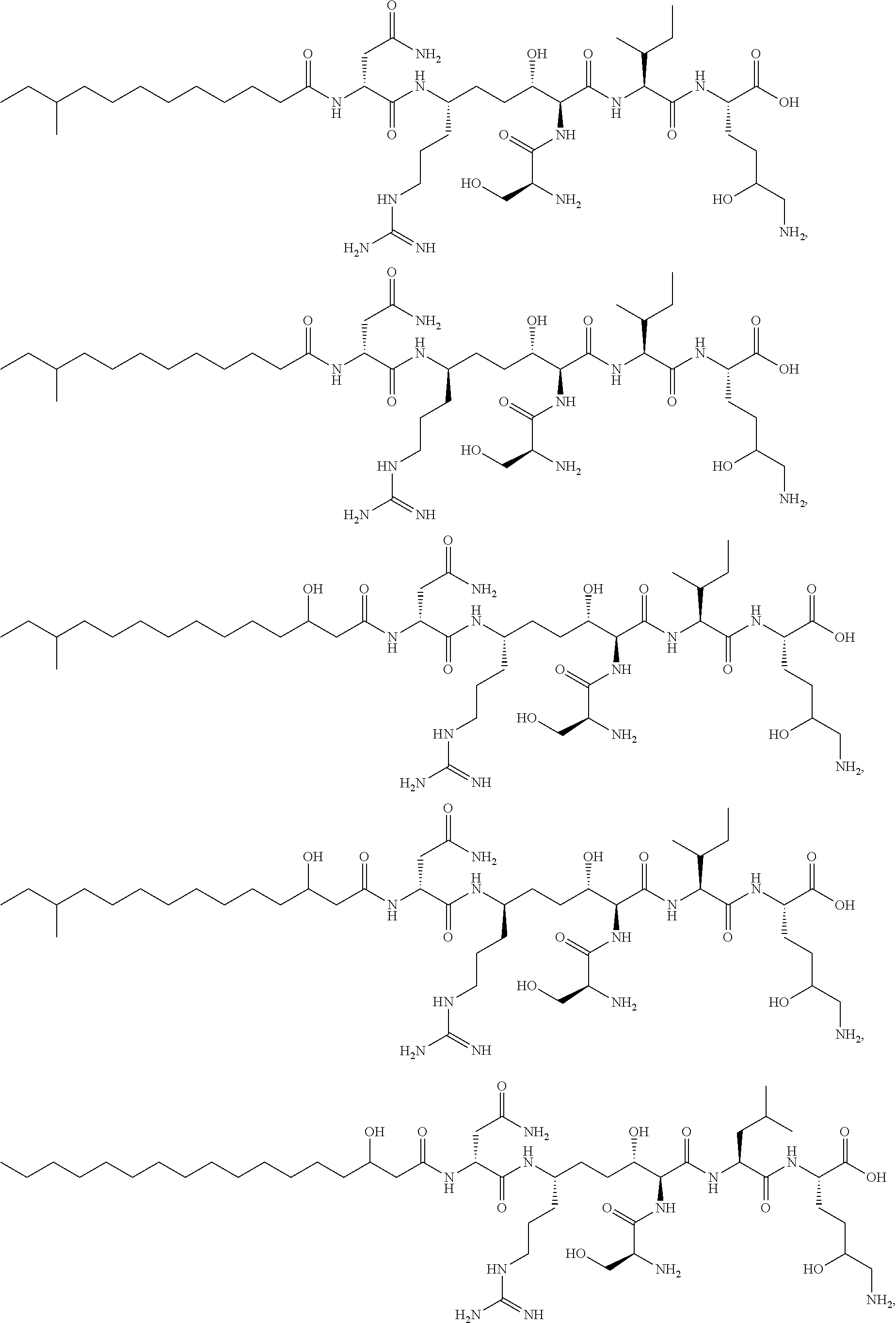

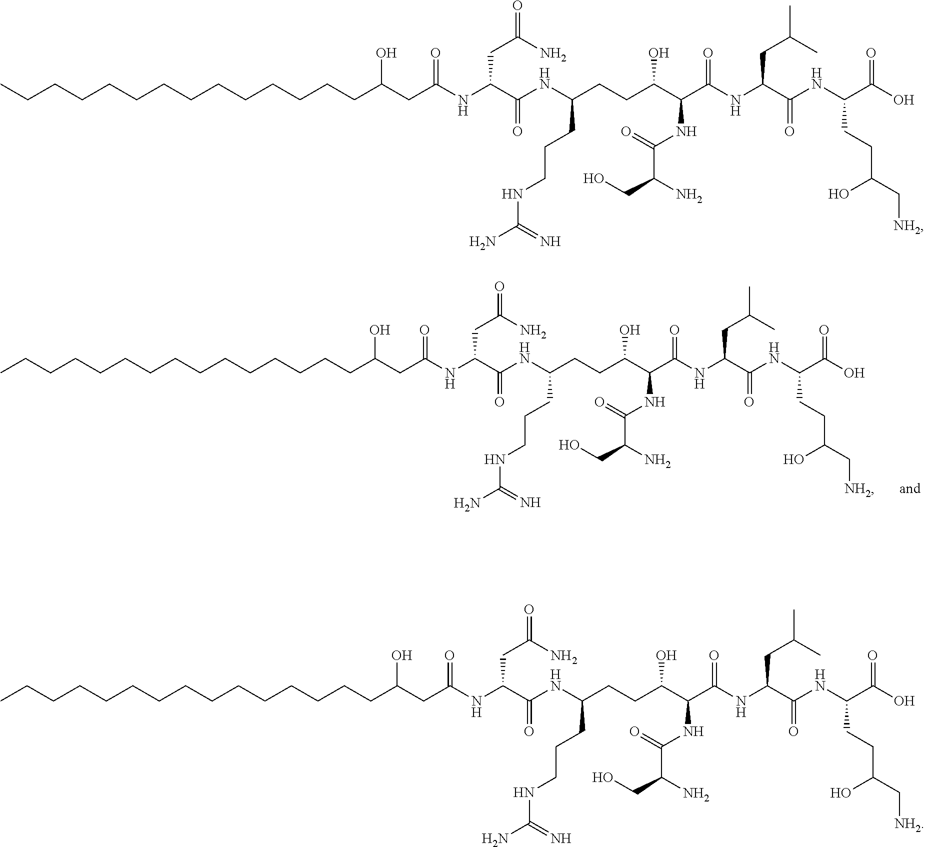 [SMILES: CCCCCCCCCCCCCCC(O)CC(=O)N[C@H](CC(N)=O)C(=O)N[C@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O ; CCCCCCCCCCCCCCCC(O)CC(=O)N[C@H](CC(N)=O)C(=O)N[C@@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O ; CCCCCCCCCCCCCCCC(O)CC(=O)N[C@H](CC(N)=O)C(=O)N[C@H](CCCNC(=N)N)CC[C@H](O)[C@H](NC(=O)[C@@H](N)CO)C(=O)N[C@@H](CC(C)C)C(=O)N[C@@H](CCC(O)CN)C(=O)O]

14. The method according to claim 1, wherein the method is performed in vivo in a subject in need of treatment.

15. The method according to claim 14, wherein the subject in need of treatment has colitis, irritable bowel syndrome (IBS), an inflammatory bowel disorder, or has a genetic predisposition to colorectal cancer.

16. A compound having the following structure: 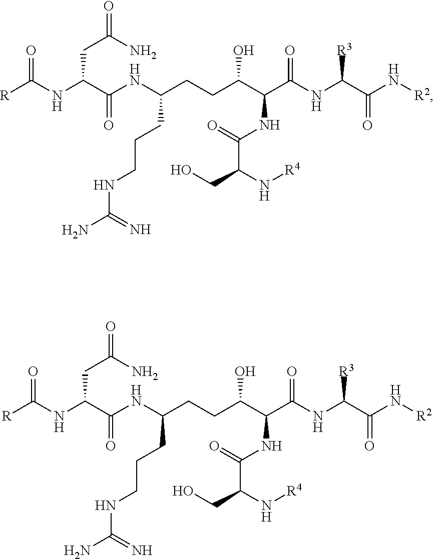
or isomers, stereoisomers, enantiomers, diastereomers, salts, hydrates, or solvates of any one or more of the foregoing, or any combination of any of the foregoing,
wherein
R is a substituted or unsubstituted aliphatic group;
R² is an amino acid residue, a substituted or unsubstituted aliphatic group, or H;
R³ is a substituted or unsubstituted aliphatic group or an amino acid side chain; and
R⁴ is a substituted or unsubstituted acyl group, an amino acid residue, or H, with the proviso the compound does not have the following structure:

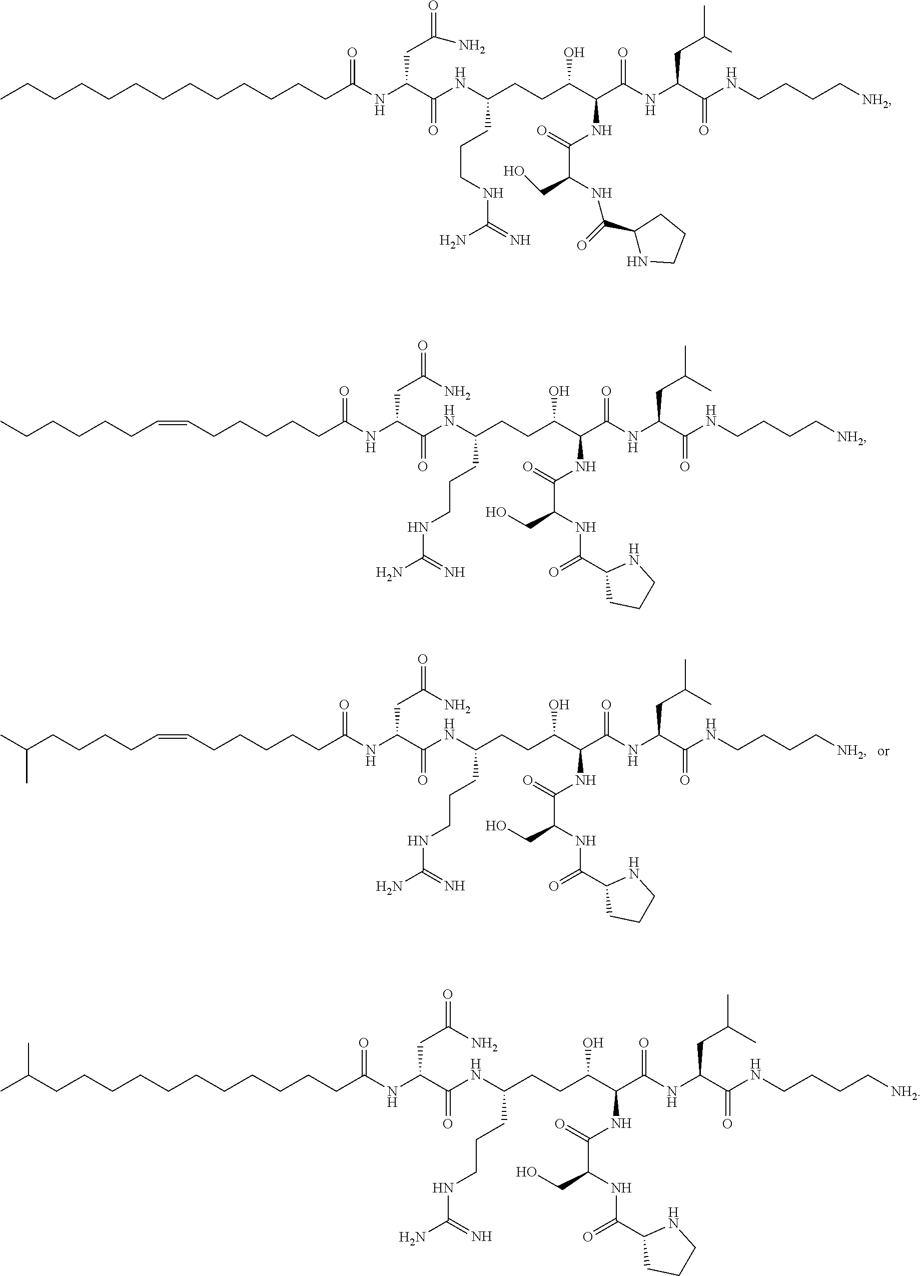

17. The compound according to claim 16, wherein the compound has the following structure:

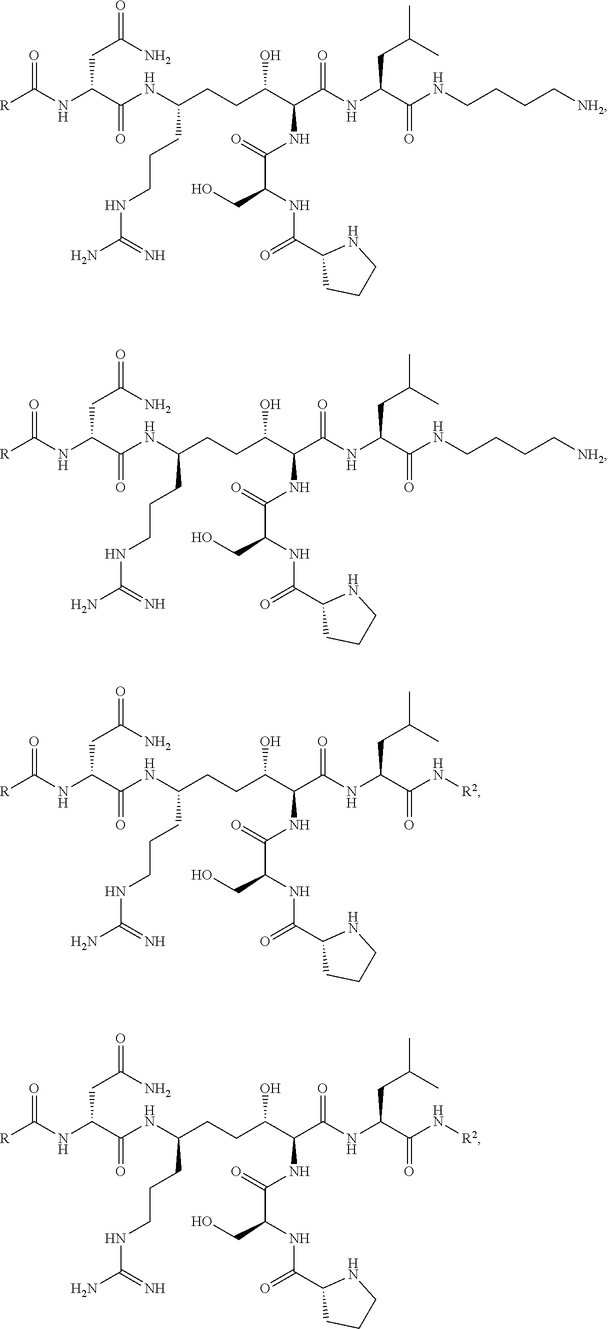

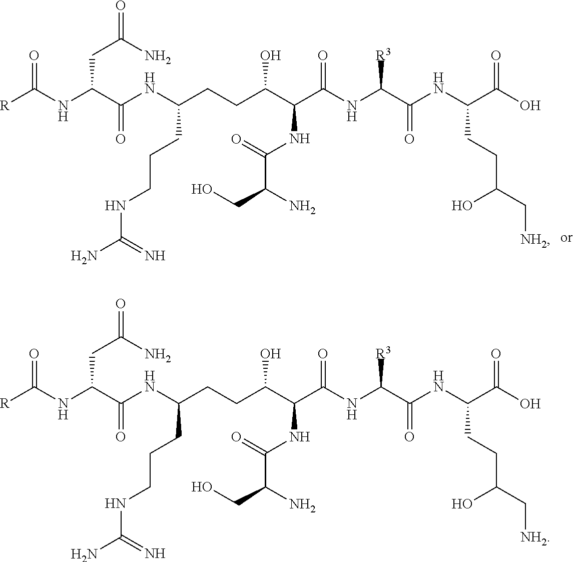

18. A compound according to claim 16, wherein R has the following structure 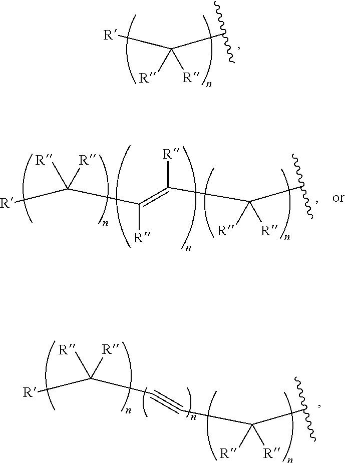
wherein R′ is hydrogen, a halogen, an aryl group, a methyl group, a halogenated methyl group, a hydroxy, and each R″ is independently hydrogen, a halogen, an aryl group, a methyl group, halogenated methyl group, or a hydroxy.

19. The compound according to claim 14, wherein R has the following structure: 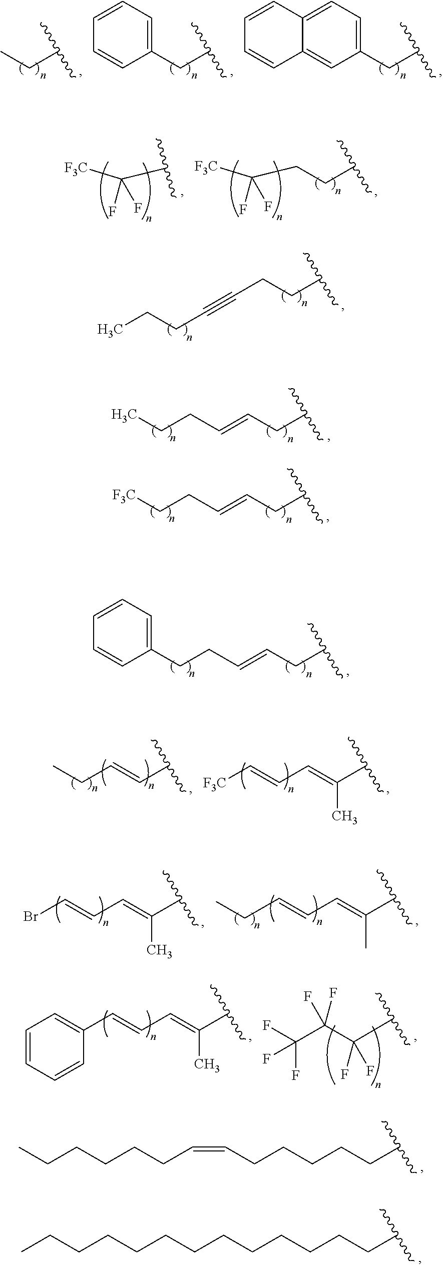 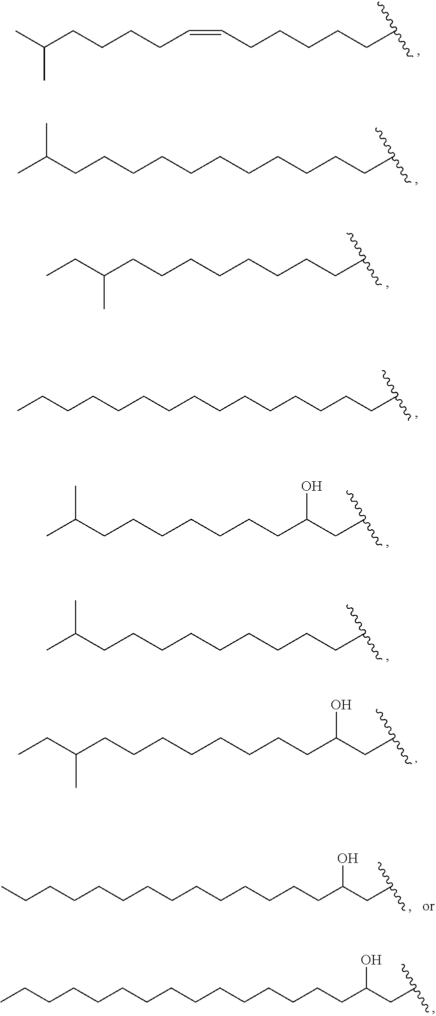
wherein each n is independently 0 to 18.

20. The compound according to claim 16, wherein R² is

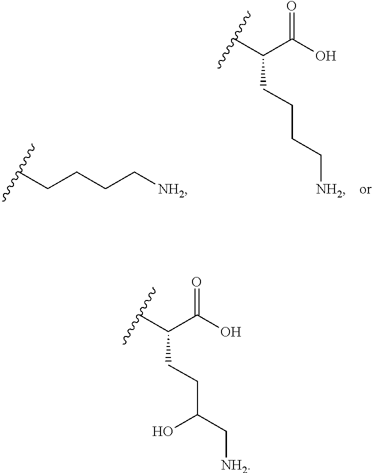 [SMILES: *CCCCN ; *[C@@H](CCCCN)C(=O)O ; *[C@@H](CCC(O)CN)C(=O)O]

21. The compound according to claim 16, wherein R³ is

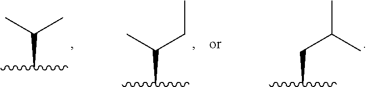

22. The compound according to claim 16, wherein R⁴ is H or

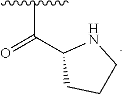 [SMILES: *C(=O)[C@H]1CCCN1]

23. A composition comprising a compound according to claim 16 and a pharmaceutically acceptable carrier/excipient.


SEQUENCE LISTING (48 sequences)

SEQ ID NO: 1 - DNA, 24 bp, synthetic construct
  source 1..24: mol_type = other DNA
cagcactgacccttttgggaccgc

SEQ ID NO: 2 - DNA, 21 bp, synthetic construct
  source 1..21: mol_type = other DNA
ctctttctcccctttatgatc

SEQ ID NO: 3 - DNA, 24 bp, synthetic construct
  source 1..24: mol_type = other DNA
aggacacactttaacaataggcga

SEQ ID NO: 4 - DNA, 21 bp, synthetic construct
  source 1..21: mol_type = other DNA
gattagcgcgcccaaacaatg

SEQ ID NO: 5 - DNA, 19 bp, synthetic construct
  source 1..19: mol_type = other DNA
cgtcggggctagttgtact

SEQ ID NO: 6 - DNA, 22 bp, synthetic construct
  source 1..22: mol_type = other DNA
aatagacggcatgtaaaccgga

SEQ ID NO: 7 - DNA, 21 bp, synthetic construct
  source 1..21: mol_type = other DNA
aggactgcgtcaaggtgcatt

SEQ ID NO: 8 - DNA, 21 bp, synthetic construct
  source 1..21: mol_type = other DNA
ttttggaacagggtaacggca

SEQ ID NO: 9 - DNA, 20 bp, synthetic construct
  source 1..20: mol_type = other DNA
ggaaagaggacaggacaacg

SEQ ID NO: 10 - DNA, 23 bp, synthetic construct
  source 1..23: mol_type = other DNA
cctcttaacacacaacgtccata

SEQ ID NO: 11 - DNA, 23 bp, synthetic construct
  source 1..23: mol_type = other DNA
tccagctgagatctcctagctag

SEQ ID NO: 12 - DNA, 20 bp, synthetic construct
  source 1..20: mol_type = other DNA
ggcgagtgttaaggtgtaca

SEQ ID NO: 13 - DNA, 19 bp, synthetic construct
  source 1..19: mol_type = other DNA
agcacttgtctaaccacca

SEQ ID NO: 14 - DNA, 19 bp, synthetic construct
  source 1..19: mol_type = other DNA
caatcgtcggcctaggaat

SEQ ID NO: 15 - DNA, 47 bp, synthetic construct
  source 1..47: mol_type = other DNA
ttgcctaacgggctgtagtagatcataaaggggagaaagagatcata

SEQ ID NO: 16 - DNA, 50 bp, synthetic construct
  source 1..50: mol_type = other DNA
gcggctgaaacacctgtgatattcagcactgacccttttgggaccgctga

SEQ ID NO: 17 - DNA, 52 bp, synthetic construct
  source 1..52: mol_type = other DNA
ccttttagaggtctccataataggtactagttgaggaatactctggcatcgg

SEQ ID NO: 18 - DNA, 48 bp, synthetic construct
  source 1..48: mol_type = other DNA
gttacggaggaaaaagcgggagccgcccctaagcgaattctagtaacg

SEQ ID NO: 19 - DNA, 47 bp, synthetic construct
  source 1..47: mol_type = other DNA
tagaattcgcttaggggcggctcccgctttttcctccgtaacgatag

SEQ ID NO: 20 - DNA, 44 bp, synthetic construct
  source 1..44: mol_type = other DNA
atttcgttttccttcaagtcgtactacttcgcctctggcatcgg

SEQ ID NO: 21 - DNA, 46 bp, synthetic construct
  source 1..46: mol_type = other DNA
agtcattattcaactggtcaaaggtaacgatcggaggacaatcggg

SEQ ID NO: 22 - DNA, 46 bp, synthetic construct
  source 1..46: mol_type = other DNA
aaatgggcggaacttgccacacgtctaaatttacaaatttgccgcg

SEQ ID NO: 23 - DNA, 51 bp, synthetic construct
  source 1..51: mol_type = other DNA
caaagtcgtccaccactaagatttacgtctaaatttacaaatttgccgcgg

SEQ ID NO: 24 - DNA, 58 bp, synthetic construct
  source 1..58: mol_type = other DNA
tatgattgaagaatgcaagtctatgtaagtaatacgatcggaggacaatcgggttttt

SEQ ID NO: 25 - DNA, 51 bp, synthetic construct; identical to SEQ ID NO: 23
  source 1..51: mol_type = other DNA

SEQ ID NO: 26 - DNA, 44 bp, synthetic construct
  source 1..44: mol_type = other DNA
agaatgcgagtctatataagtaccttaaggaggacaatcgggtt

SEQ ID NO: 27 - DNA, 50 bp, synthetic construct
  source 1..50: mol_type = other DNA
gcgacgtgactgagtaggtattactgttaattagtagccgagcatattac

SEQ ID NO: 28 - DNA, 42 bp, synthetic construct
  source 1..42: mol_type = other DNA
accgataatagaataacccaaagttttcaatacctactcagt

SEQ ID NO: 29 - DNA, 53 bp, synthetic construct
  source 1..53: mol_type = other DNA
gccggtcccaaaaatatgcataattactgttaattagtagccgagcatattac

SEQ ID NO: 30 - DNA, 30 bp, synthetic construct
  source 1..30: mol_type = other DNA
gtgtaatatgctcggctactaattaacagt

SEQ ID NO: 31 - DNA, 29 bp, synthetic construct
  source 1..29: mol_type = other DNA
gcaaaaaaacccgattgtcctccttaagg

SEQ ID NO: 32 - DNA, 50 bp, synthetic construct
  source 1..50: mol_type = other DNA
cggtcacgtaattcctagtgtagacgtccagctgagatctcctagctagg

SEQ ID NO: 33 - DNA, 47 bp, synthetic construct
  source 1..47: mol_type = other DNA
ttctcccgccttgagtactaattaaagaaaaaggagtggtactttag

SEQ ID NO: 34 - DNA, 49 bp, synthetic construct
  source 1..49: mol_type = other DNA
tgacctaaagtaccactcctttttctttaattagtactcaaggcgggag

SEQ ID NO: 35 - DNA, 51 bp, synthetic construct
  source 1..51: mol_type = other DNA
ataggactttcagagtccctacacctagactcgagagggcccttaaggtgt

SEQ ID NO: 36 - DNA, 51 bp, synthetic construct
  source 1..51: mol_type = other DNA
caactcttactgtagttttccggacgtccagctgagatctcctagctagga

SEQ ID NO: 37 - DNA, 41 bp, synthetic construct
  source 1..41: mol_type = other DNA
agttggcacaggcgtcctcaaggcagggcattttcgattat

SEQ ID NO: 38 - DNA, 41 bp, synthetic construct
  source 1..41: mol_type = other DNA
ataatcgaaaatgccctgccttgaggacgcctgtgccaact

SEQ ID NO: 39 - DNA, 49 bp, synthetic construct
  source 1..49: mol_type = other DNA
cggtggacgtattggtaataccctagactcgagagggcccttaaggtgt

SEQ ID NO: 40 - DNA, 23 bp, synthetic construct
  source 1..23: mol_type = other DNA
tccagctgagatctcctagctag

SEQ ID NO: 41 - DNA, 20 bp, synthetic construct
  source 1..20: mol_type = other DNA
ggcgagtgttaaggtgtaca

SEQ ID NO: 42 - DNA, 60 bp, synthetic construct
  source 1..60: mol_type = other DNA
ttttagtcaataacgaaaacttttattcccgtatggtggttagacaagtgctacccggag

SEQ ID NO: 43 - DNA, 53 bp, synthetic construct
  source 1..53: mol_type = other DNA
accagagaggactaagttgattaactattcctaggccgacgattgtttcgggc

SEQ ID NO: 44 - DNA, 56 bp, synthetic construct
  source 1..56: mol_type = other DNA
ttttagtcaataacgaaaacttttattcccgtagatcataaaggggagaaagagat

SEQ ID NO: 45 - DNA, 49 bp, synthetic construct
  source 1..49: mol_type = other DNA
accagagaggactaagttgattaactcagcactgacccttttgggaccg

SEQ ID NO: 46 - DNA, 669 bp, synthetic construct
  source 1..669: mol_type = other DNA
ccgatgccagagtgaatggcgtagtttctcacacacattaaagtgaaagagatgcgtttctcccgctaaaggcaaacggagttgttagaggtcggtgacaacacactcaactatctctcttactgagagaagttggaagtctgcactaggacagtagcagacagactgcaaaaaggcttatgctctgttaacaagatagcggcgaccgagacattaacgtccatcgaaactcgacatttccacctttattgtcataggacttttgacaaagttggctaagagaacgcacgaattacggacttacctccacttgtaaagtaggcggggggtgtcgcttgtgtatccgtcggggcgtgcaactcactctattgagtcgtgtaaaaatccgctctgaaggtctattgcccagtgtggtaaaacgcgttgcatgacattcgagccattggtaacgtggcctctttattctacaacgtcttagctacaagactctaaaaggcgtccattgtggttcaaccattaaccggattgttaacgcgtctttccgggtgggcttccactcggtcaccggaataatttactgaagagtagatgttgtcatctttaataaatttcaagaatctgcaacactaacaacatagactacggtgtgcaatagaactctggcatcgg

SEQ ID NO: 47 - DNA, 688 bp, synthetic construct
  source 1..688: mol_type = other DNA
ccgatgccagagctctgactagaattcgcttaggggcgggcgtagctttagatgttgtcatctttaataaatttcaagaatctgaaatcgaaggaatcgaggacttttagagcttgatcataatatggatcctgactcgatcgacagttcctaggtcgtatacgccacaccctaggagttaaagtacgccattcgctaaacgcggcgtttgcttcggcctagcaatattaaaggctatagcttgtaaagactccataggactgaccgaggccatttaacgggtttagcccagtctaagttacaccttggttacttttgtggacgtaaatcgtaacgtagaggccctcagtaaggacgtagttcctttcgtaactttgactgaagtacgtcgtgggcttcgacacccaggtagaaggcacgaagggcaacgcttatcggctttttcgctattagtttcgtaagtcatagaagttcccgccgaaaccgaccgtgtattaacccaaccagttattggaggacgccctacggttatcggttttcgagaagcgctcttttcttgctatggttaagggcgggacggtcgttcaaaacggtttttgcgacggttaggcccgatacccctttggctgtcctttacggttattagcgttgaagtaatggccatataagtgcgaacctctggcatcgg

SEQ ID NO: 48 - DNA, 58 bp, synthetic construct; identical to SEQ ID NO: 24
  source 1..58: mol_type = other DNA